(19) United States
(12) Patent Application Publication
(10) Pub. No.: US 2023/0329201 A1
(43) Pub. Date: Oct. 19, 2023

(54) CELLS AND NON-HUMAN ANIMALS ENGINEERED TO EXPRESS ADAR1 AND USES THEREOF

(71) Applicant: WAVE LIFE SCIENCES LTD., Singapore (SG)

(72) Inventors: Hailin Yang, West Roxbury, MA (US); Prashant Monian, Arlington, MA (US); Chikdu Shakti Shivalila, Woburn, MA (US); Subramanian Marappan, Acton, MA (US); Chandra Vargeese, Schwenksville, PA (US); Pachamuthu Kandasamy, Lexinnton, MA (US); Genliang Lu, Winchester, MA (US); Hui Yu, Arlington, MA (US); David Charles Donnell Butler, Medford, MA (US); Luciano Henrique Apponi, Lynn, MA (US); Mamoru Shimizu, Arlington, MA (US); Stephany Michelle Standley, Wakefield, MA (US); David John Boulay, Cambridge, MA (US); Jack David Godfrey, Arlington, MA (US); Naoki Iwanmoto, Boston, MA (US)

(21) Appl. No.: 18/022,509

(22) PCT Filed: Aug. 23, 2021
(86) PCT No.: PCT/US2021/047205

§ 371 Date: Feb. 21, 2023

Related U.S. Application Data
(60) Provisional application No. 63/175,031, filed on Apr. 14, 2021.
(60) Provisional application No. 63/111,072, filed on Nov. 8, 2020.
(60) Provisional application No. 63/069,698, filed on Aug. 24, 2020.

(51) Int. Cl. A01K 67/027; C12N 15/85; C12N 9/78; A61K 49/00

(52) U.S. Cl. CPC A01K 67/0275; A61K 49/0008; C12N 9/78; C12N 15/8509; A01K 2217/072; A01K 2227/105; A01K 2267/01; C12N 2015/8518

(57) ABSTRACT

Among other things, the present disclosure provides cells and non-human animals engineered to express an ADAR1 polypeptide or a characteristic portion thereof. In some embodiments, the present disclosure provides cells and non-human animals engineered to express a human ADAR1 polypeptide or a characteristic portion thereof. In some embodiments, non-human animals are genetically modified rodents such as mice, rat, etc. In some embodiments, non-human animals are mice. In some embodiments, the present disclosure provides technologies for assessing an agent comprising administering the agent to a cell or non-human animal engineered to express an ADAR1 polypeptide or a characteristic portion thereof. In some embodiments, such a cell or non-human animal is engineered to express a human ADAR1 polypeptide or a characteristic portion thereof. In some embodiments, an agent is a pharmaceutical agent. In some embodiments, an agent is or comprises an oligonucleotide.

### CROSS-REFERENCE TO RELATED APPLICATIONS

This application claims priority to U.S. Provisional Application Nos. 63/069,698, filed Aug. 24, 2020, 63/111,072, filed Nov. 08, 2020, and 63/175,031, filed Apr. 14, 2021, the entirety of each of which is incorporated herein by reference.

### BACKGROUND

Oligonucleotides are useful in various applications, e.g., therapeutic, diagnostic, and/or research applications. For example, oligonucleotides targeting various genes can be useful for treatment of conditions, disorders or diseases related to such target genes.

### SUMMARY

Among other things, the present disclosure provides cells, embryos, and non-human animals engineered to comprise and/or express an ADAR1 polypeptide or a characteristic portion thereof. In some embodiments, embryos, and non-human animals engineered to comprise and/or express a polynucleotide whose sequence encodes an ADAR1 polypeptide or a characteristic portion thereof.

In some embodiments, cells are rodent e.g., mouse, cells. In some embodiments, embryos are rodent, e.g., mouse, embryos. In some embodiments, a non-human animal is a rodent. In some embodiments, it is a rat. In some embodiments, it is a mouse.

In some embodiments, an ADAR1 polypeptide or a characteristic portion thereof is or comprises a primate (e.g., human) ADAR1 polypeptide or a characteristic portion thereof. In some embodiments, an ADAR1 polypeptide or a characteristic portion thereof is or comprises a human ADAR1 p110 polypeptide or a characteristic portion thereof. In some embodiments, an ADAR1 polypeptide or a characteristic portion thereof is or comprises a human ADAR1 p150 polypeptide or a characteristic portion thereof. In some embodiments, an ADAR1 polypeptide or a characteristic portion thereof is or comprises human ADAR1. In some embodiments, an ADAR1 polypeptide or a characteristic portion thereof is or comprises a human ADAR1 p110 peptide. In some embodiments, an ADAR1 polypeptide or a characteristic portion thereof is or comprises a human ADAR1 p150 peptide. In some embodiments, an ADAR1 polypeptide or a characteristic portion thereof is or comprises one or more or all of the following domains of a primate (e.g., human) ADAR1: Z-DNA binding domains, dsRNA binding domains, and deaminase domain. In some embodiments, an ADAR1 polypeptide or a characteristic portion thereof is or comprises one or both of a primate (e.g., human) ADAR1 Z-DNA binding domains; alternatively or additionally, in some embodiments, an ADAR1 polypeptide or a characteristic portion thereof is or comprises one, two or all of a primate (e.g., human) ADAR1 dsRNA binding domains; alternatively or additionally, an ADAR1 polypeptide or a characteristic portion thereof is or comprises a primate (e.g., human) deaminase domain. In some embodiments, a primate (e.g., human) ADAR1 polypeptide or a characteristic portion thereof may be expressed together with a non-primate (e.g., a rodent such as a mice) ADAR1 polypeptide or a characteristic portion thereof, e.g., one or more human dsRNA binding domains may be engineered to be expressed together with a mouse ADAR1 deaminase domain to form a human-mouse hybrid ADAR1 polypeptide. In some embodiments, an ADAR1 polypeptide or a characteristic portion thereof is or comprises a non-primate (e.g., rodent (e.g., mouse)) ADAR1, wherein a non-primate ADAR1 is engineered to have one or more of its domains replaced with one or more corresponding primate (e.g., human) ADAR1 domains (e.g., Z-DNA binding domains, dsRNA binding domains, and/or deaminase domains).

Among other things, provided technologies (e.g., cells, embryos, animals, methods, etc.) are useful for assessing various agents whose activities may be associated with ADAR1. For example, in some embodiments, provided technologies are particularly useful as animal models for assessing/characterizing various agents, e.g., oligonucleotides, and compositions thereof, for nucleic acid editing, e.g., adenosine editing in transcripts (e.g., A to I conversion). Among other things, the present disclosure encompasses the recognition that various agents (e.g., oligonucleotides) and compositions thereof that can provide editing in various human systems, e.g., cells, may show no or much lower levels of editing in certain cells (e.g., rodent cells such as mouse cells) and certain animals such as rodents (e.g., mice) that do not contain or express human ADAR1. Particularly, mice, a commonly used animal model, may be of limited uses for assessing various agents (e.g., oligonucleotides) for editing in humans, as various agents active in human cells provide no or very low levels of activity in mouse cells and animals not engineered to comprise or express a proper ADAR1 (e.g., human ADAR1) polypeptide or a characteristic portion thereof (see FIGS. 22-26, data for wild-type (WT) mice and cells, human cells, and cells and mice engineered to express hADAR1 p110 (huADAR mouse)). In some embodiments, the present disclosure provides cells and non-human animals (e.g., rodents such as mice) engineered to express an ADAR1 polypeptide or a characteristic portion thereof (e.g., human ADAR1 p110, p150, etc.), and their uses for assessing/characterizing editing agents such as various oligonucleotides and compositions thereof. Among other things, such engineered cells and/or animals can demonstrate activities that are more correlated with and/or predictive of activities in human cells than cells and/or animals not so engineered.

In some embodiments, engineered cells, embryos, non-human animals, etc., are genetically modified. In some embodiments, engineered cells, embryos, non-human animals comprise a polynucleotide whose sequence encodes an ADAR1 polypeptide or a characteristic portion thereof as described herein. In some embodiments, genomes of engineered cells, embryos, non-human animals comprise a polynucleotide whose sequence encodes an ADAR1 polypeptide or a characteristic portion thereof as described herein. In some embodiments, germline genomes of engineered non-human animals comprise a polynucleotide whose sequence encodes an ADAR1 polypeptide or a characteristic portion thereof as described herein.

In some embodiments, the present disclosure provides genetically modified rodents. In some embodiments, a genetically modified rodent as provided is a rat or a mouse. In some embodiments, all endogenous sequences are rat or mouse sequences. For example, in some embodiments, a genetically modified rodent is a mouse and all endogenous sequences are mouse sequences. In some embodiments, a genetically modified rodent is a rat and all endogenous sequences are rat sequences.

In some embodiments, the present disclosure provides a breeding colony of genetically modified rodents provided herein comprising a first genetically modified rodent, a second genetically modified rodent, and a third genetically modified rodent, where the first, second, and third genetically modified rodent are each a genetically modified rodent as described herein. In some embodiments, a third genetically modified rodent is the progeny of a first genetically modified rodent and a second genetically modified rodent.

In some embodiments, engineered cells, embryos, non-human animals, etc. are heterozygous. In some embodiments, engineered cells, embryos, non-human animals, etc. are homozygous.

In some embodiments, the present disclosure provides technologies for making engineered cells, embryos, non-human animals, etc. In some embodiments, the present disclosure provides technologies for assessing/characterizing engineered cells, embryos, non-human animals, etc.

### BRIEF DESCRIPTION OF THE DRAWING

The Drawing included herein, which is composed of the following Figures, is for illustration purposes only and not for limitation.

FIG. 1 shows illustrations of an exemplary embodiment, not to scale, of a strategy for constructing a targeting vector (described in Example 1) used in generating an embodiment of a non-human animal according to the present disclosure. “LR” represents the 5′ homology arm targeting the ROSA26 locus represented by SEQ ID NO: 62, “Adenovirus splice acceptor” represents a splice acceptor as represented by SEQ ID NO: 57, “CDS” represents an ADAR1 p110 locus sequence represented by SEQ ID NO: 14, “WPRE” represents the Woodchuck Hepatitis Virus Posttranscriptional Regulatory Element represented by SEQ ID NO: 56, “bGH poly(A) signal” represents the bovine growth hormone poly(A) signal represented by SEQ ID NO: 59, “RR” represents the 3′ homology arm targeting the ROSA26 locus represented by SEQ ID NO: 63, “Ori” represents the plasmid origin of replication, and “AmpR” represents the ampicillin resistance gene.

FIG. 2 shows illustrations of an exemplary embodiment, not to scale, of a strategy for constructing a targeting vector (described in Example 1) used in generating an embodiment of a non-human animal according to the present disclosure. “LR” represents the 5′ homology arm targeting the ROSA26 locus represented by SEQ ID NO: 62, “Adenovirus splice acceptor” represents a splice acceptor as represented by SEQ ID NO: 57, “CDS” represents an ADAR1 p150 locus sequence represented by SEQ ID NO: 3, “WPRE” represents the Woodchuck Hepatitis Virus Posttranscriptional Regulatory Element represented by SEQ ID NO: 56, “bGH poly(A) signal” represents the bovine growth hormone poly(A) signal represented by SEQ ID NO: 59, “RR” represents the 3′ homology arm targeting the ROSA26 locus represented by SEQ ID NO: 63, “Ori” represents the plasmid origin of replication, and “AmpR” represents the ampicillin resistance gene.

FIG. 3 depicts certain data for a series of guide RNAs for targeting a mouse ROSA26 locus. Relative Cas9/sgRNA activity for sgRNA molecules was determined using luciferase interruption assays. Sg12 selected for further use in transgenic animal creation.

FIG. 4 depicts a targeting scheme for the introduction of targeting vector A (EGE-JGY-045-CDS-p110) into a WT ROSA26 allele. The 5′ and 3′ homology arms are complementary to the targeted allele.

FIG. 5 depicts certain restriction enzyme digestion and southern blot strategies for construct EGE-JGY-045-A (targeting vector comprising huADAR1 p110, as represented by SEQ ID NO: 64). Various parallel restriction enzyme digestion assays are used to confirm the correct incorporation of the ADAR1 polynucleotide. In addition, 5′ and 3′ (WPRE probe) southern blot probes are designed to confirm genotyping results following targeting vector integration into the ROSA26 locus.

FIG. 6 depicts certain restriction enzyme digestion results for the confirmation of correct cloning for targeting vector A construct #6. Three restriction enzyme digestion assays were performed in parallel, comprising 1) restriction enzymes XhoI and BamHI, wherein successful cloning and digestion produces products 7069 bp and 3039 bp in length; 2) restriction enzyme SalI, wherein successful cloning and digestion produces products 4765 bp, 3255 bp and 2115 bp in length; 3) restriction enzyme ScaI, wherein successful cloning and digestion produces products 7093 bp and 3042 bp in length. Construct #6 was utilized for additional cloning and intergenic introduction of human ADAR1 (huADAR1) p110 into the mouse genome.

FIG. 7 depicts a primer design for screening for successful huADAR1 p110 integration events. For PCR primer set sequences and predicted product sizes, see Table 2.

FIG. 8 depicts a primer design for confirmation of successful huADAR1 p110 integration events. For PCR primer set sequences and predicted product sizes, see Table 2.

FIG. 9 depicts initial founder genotyping for huADAR1 p110 integration events in a ROSA26 locus. A: PCR products from four potential founder animals EY744-005, -008, -0036, and -0037, each of which display an appropriately sized 2219 bp product matching the predicted product size. B: PCR products from four potential founder animals EY74-005, -008, -0036, and -0037, each of which display an appropriately sized 2221 bp product matching the predicted product size. C: PCR products from four potential founder animals EY74-005, -008, -0036, and -0037, each of which display an appropriately sized 3191 bp product matching the predicted product size. For PCR primer set sequences and predicted product sizes, see Table 2.

FIG. 10 depicts F1 genotyping for presence of integrated huADAR1 p110 following founder animal crosses. A: PCR products from five potential huADAR1 p110 F1 mice, 1E7Y45-00010, -0002, -0003, -004, and -0013, each of which display an appropriately sized 2219 bp product matching the predicted product size. B: PCR products from five potential huADAR1 p110 F1 mice, 1E7Y45-00010, -0002, -0003, -004, and -0013, each of which display an appropriately sized 2221 bp product matching the predicted product size. C: PCR products from five potential huADAR1 p110 F1 mice, 1E7Y45-00010, -0002, -0003, -004, and -0013, each of which display an appropriately sized 3191 bp product matching the predicted product size. D: PCR products from five potential huADAR1 p110 F1 mice, 1E7Y45-00010, -0002, -0003, -004, and -0013, each of which display an appropriately sized 469 bp product matching the predicted product size and suggesting heterozygosity. E: PCR products from five potential huADAR1 p110 F1 mice, 1E7Y45-00010, -0002, -0003, -004, and -0013, each of which display an appropriately sized 264 bp product matching the predicted product size and suggesting heterozygosity. For PCR primer set sequences and predicted product sizes, see Table 2.

FIG. 11 depicts southern blot strategy results confirming proper integration of ADAR1 polynucleotide. In southern blot analysis, presence of a huADAR1 p110 transgene is identified by the 5′ probe at 9.9 kb, and the 3′ probe at 4.7 kb (described in 11A, depicted in 11B). The additional presence of the 6.1 kb 5′ probe confirms the heterozygosity of the F1 animals.

FIG. 12 depicts restriction enzyme digestion and southern blot strategies for construct EGE-JGY-046-A (targeting vector comprising huADAR1 p150, as represented by SEQ ID NO: 65). Various parallel restriction enzyme digestion assays are used to confirm the correct incorporation of the ADAR1 polynucleotide. In addition, 5′ and 3′ (WPRE probe) southern blot probes are designed to confirm genotyping results following targeting vector integration into the ROSA26 locus.

FIG. 13 depicts the restriction enzyme digestion results for the identification of correct clones for targeting vector B constructs. A: an initial three restriction enzyme digestion assays that were performed in parallel, comprising 1) restriction enzymes BamHI and SacI, wherein successful cloning and digestion produces products 5452bp, 4214bp, and 1354bp in length (represented by constructs #5 and #6); 2) restriction enzymes XhoI and MluI, wherein successful cloning and digestion produces products 6196bp and 4824bp in length (represented by constructs #5 and #6); and 3) restriction enzyme SalI, wherein successful cloning and digestion produces products 4765bp, 4140, and 2115 in length (represented by constructs #5 and #6). Construct #5 was utilized for additional restriction enzyme confirmation. B: three confirmatory restriction enzyme digestions following plasmid amplification, comprising 1) restriction enzymes BamHI and SacI, wherein successful cloning and digestion produces products 5452, 4214, and 1354 in length; 2) restriction enzymes XhoI and MluI, wherein successful cloning and digestion produces products 6196 and 4824 in length; and 3) restriction enzymes NdeI and KpnI, wherein successful cloning and digestion produces products 5405, 3244, and 2371 in length. Construct #5 was selected for intergenic introduction of human ADAR1 (huADAR1) p150 into the mouse genome.

FIG. 14 depicts a primer design for screening for successful huADAR1 p150 integration events. For PCR primer set sequences and predicted product sizes, see Table 2.

FIG. 15 depicts a primer design for confirmation of successful huADAR1 p150 integration events. For PCR primer set sequences and predicted product sizes, see Table 2.

FIG. 16 depicts initial founder genotyping for huADAR1 p150 integration events in the ROSA26 locus. A: PCR products from seven potential founder animals EY746-005, -0012, -0016, -0024, 0030,0051, and -0054, each of which display an appropriately sized 2211 bp product matching the predicted product size. B: depicts PCR products from seven potential founder animals EY746-005, -0012, 016,0024, -0030, -0051, and -0054, each of which display an appropriately sized 2221 bp product matching the predicted product size. C: depicts PCR products from seven potential founder animals EY746-005, -0012, -0016, -0024, -0030, -0051, and -0054, each of which display an appropriately sized 1521 bp product matching the predicted product size. D: depicts PCR products from seven potential founder animals EY746-005, -0012, -0016, -0024, -0030, -0051, and -0054, each of which display an appropriately sized 2719 bp product matching the predicted product size. For PCR primer set sequences and predicted product sizes, see Table 2.

FIG. 17 depicts F1 genotyping for presence of integrated huADAR1 p150 following founder animal crosses. A: PCR products from 17 potential F1 transgenic animals: 1EY746-007, -0013, -0015, -0016, -0019, -0021, -0024, -0030, -0032, -0033, -0035, -0047, -0048, -00580, -0064, -0065, and -0070, most of which display an appropriately sized 2211 bp product matching the predicted product size. B: PCR products from 17 potential F1 transgenic animals: 1EY746-007, -0013, -0015, -0016, -0019, -0021, -0024, -0030, -0032, -0033, -0035, -0047, -0048, -00580, -0064, -0065, and -0070, most of which display an appropriately sized 2221 bp product matching the predicted product size. C: PCR products from 17 potential F1 transgenic animals: 1EY746-007, -0013, -0015, -0016, -0019, -0021, -0024, -0030, -0032, 0033,0035, -0047, -0048, -00580, -0064, -0065, and -0070, most of which display an appropriately sized 1512 bp product matching the predicted product size. D: PCR products from 17 potential F1 transgenic animals: 1EY746-007, -0013, -0015, -0016, -0019, -0021, -0024, -0030, -0032, -0033, -0035, -0047, -0048, -00580, -0064, -0065, and -0070, most of which display an appropriately sized 2719 bp product matching the predicted product size. For PCR primer set sequences and predicted product sizes, see Table 2.

FIG. 18 depicts southern blot strategy (A) and results (B) confirming integration of ADAR1 polynucleotide. In southern blot analysis, presence of a huADAR1 p150 transgene is identified by the 5′ probe at 9.9 kb, and the 3′ probe at 4.7 kb. The additional presence of the 8.9 kb 5′ probe confirms the heterozygosity of the F1 animals. Results in panel B reveals that 1E7Y46-0024 contained the appropriate transgene banding pattern displaying clean heterozygosity (i.e., with no additional unidentified bands). Animal 1E7Y46-0024 was selected for further genotyping analysis by PCR.

FIG. 19 depicts supplementary F1 genotyping for presence of integrated huADAR1 p150 following founder animal crosses. A: PCR products from potential F1 transgenic animal 1EY746-0024, which displays an appropriately sized 469 bp product matching the predicted product size and suggesting heterozygosity. B: depicts PCR products from potential F1 transgenic animal 1EY746-0024, which displays an appropriately sized 256 bp product matching the predicted product size and suggesting heterozygosity. For PCR primer set sequences and predicted product sizes, see Table 2.

FIG. 20 depicts exemplary western blot analysis results confirming expression of huADAR1 p110 in transgenic mice (also labelled as hADAR). A: expression of human ADAR1 in human primary hepatocytes, lack of huADAR1 expression in WT C57BL/6J mice, and expression levels similar to human hepatocytes in huADAR1 p110 transgenic mice. GAPDH was utilized as the loading control, and analysis was done using 9 ug or 4.5 ug of normalized protein input. B: central nervous system (CNS) expression of human ADAR1 in human induced neurons (iCell iNeurons), lack of huADAR1 expression in WT C57BL/6J mice, and expression levels similar to human iNeurons in huADAR1 p110 transgenic mice across the cerebellum, pons/medulla, cortex, and midbrain. GAPDH was utilized as a loading control for normalized protein input.

FIG. 21 depicts exemplary western blot analysis results confirming expression of huADAR1 p110 in transgenic mice. Expression of huADAR1 p110 in Central nervous system (CNS) and lung of huADAR1 p110 transgenic mice, and lack of huADAR1 CNS or lung expression in WT C57BL/6J mice. GAPDH was utilized as a loading control for normalized protein input.

FIG. 22 depicts in vivo liver tissue editing levels of endogenous mouse UGP2 transcripts. Mice were dosed on day 0, day 2, and day 4 with 10 mg/kg WV-38700 or WV-38702 or control vehicle (PBS); on experimental day 6 (7 days after initial treatment exposure) liver tissue was harvested for measurement of oligonucleotide site-directed RNA editing mediated by ADAR proteins in the liver of huADAR1 p110 transgenic mice or WT C57BL/6J mice. Editing events induced by WV-38702 in huADAR1 p110 mice were detectable, while there was no detectable WV-38702 induced editing in WT C57BL/6J mice.

FIG. 23 depicts in vivo liver tissue editing levels of endogenous mouse EEF1A1 transcripts. Mice were dosed on day 0, day 2, and day 4 with 10 mg/kg WV-38697 or WV-38699 or control vehicle (PBS); on experimental day 6 (7 days after initial treatment exposure) liver tissue was harvested for measurement of oligonucleotide site-directed RNA editing mediated by ADAR proteins in the liver of huADAR1 p110 transgenic mice or WT C57BL/6J mice. Editing events induced by WV-38699 in huADAR1 p110 mice were detectable in greater abundance than WV-38699 induced editing in WT C57BL/6J mice.

FIG. 24 depicts in-vitro editing levels for UGP2 (A) or EEF1A1 (B) in human hepatocytes, WT C57BL/6J mouse primary hepatocytes, or transgenic huADARI p110 mouse primary hepatocytes. Transcripts for UGP2 or EEF1A1 were targeted using WV-38700 or WV-38702 (UGP2) or WV-38697 or WV-38699 (EEF1A1) each of which comprises GalNAc and is delivered for gymnotic uptake. A: average UGP2 editing level when dosed with 1 uM oligonucleotide. B: average EEF1A1 editing level when dosed with 1 uM oligonucleotide.

FIG. 25 depicts the in vitro editing levels for UGP2 in human hepatocytes, WT C57BL/6J mouse primary hepatocytes, or transgenic huADAR1 p110 mouse primary hepatocytes. Transcripts for UGP2 were targeted using WV-38700, WV-38701, or WV-38702 comprising GalNAc to mediate uptake. A: average UGP2 editing level when dosed with 1 uM WV-38701 or WV-38702 in human hepatocytes, WT C57BL/6J mouse hepatocytes, and huADAR1 p110 transgenic mouse hepatocytes. B: concentration dependent editing levels for WV-38700, WV-38701, and WV-38702 in human hepatocytes. C: concentration dependent editing levels for WV-38700, WV-38701, and WV-38702 in WT C57BL/6J mouse hepatocytes. D: concentration dependent editing levels for WV-38700, WV-38701, and WV-38702 in huADAR1 p110 transgenic mouse hepatocytes.

FIG. 26 depicts the in-vitro editing levels for EEF1A1 in human hepatocytes, WT C57BL/6J mouse primary hepatocytes, or transgenic huADAR1 p110 mouse primary hepatocytes. Transcripts for EEF1A1 were targeted using WV-38697, WV-38698, or WV-38699 comprising GalNAc to mediate uptake. A: average EEF1A1 editing level when dosed with 1 uM WV-38698, WV-38697 or WV-38699 in human hepatocytes, WT C57BL/6J mouse hepatocytes, and huADAR1 p110 transgenic mouse hepatocytes. B: concentration dependent editing levels for oligonucleotides WV-38697, WV-38698, or WV-38699 in human hepatocytes. C: concentration dependent editing levels for oligonucleotides WV-38697, WV-38698, or WV-38699 in WT C57BL/6J mouse hepatocytes. D: concentration dependent editing levels for oligonucleotides WV-38697, WV-38698, or WV-38699 in huADAR1 p110 transgenic mouse hepatocytes.

FIG. 27 depicts in-vivo CNS tissue (e.g., cortex, hippocampus, striatum, brain stem, cerebellum, and spinal cord) editing levels of endogenous mouse UGP2 transcripts. Mice were dosed with control vehicle (PBS) or WV-40590 at 100 ug on day 0, or 50 ug on day 0 and 50 ug on day 2. On experimental day 6 (7 days after initial treatment exposure) CNS tissue was harvested for measurement of site-directed RNA editing in the CNS of huADAR1 p110 transgenic mice. A: average CNS editing levels in huADAR1 p110 mice. No editing was induced by PBS. In some embodiments, single dosage using 100 ug generated a larger editing response than two temporally dispersed doses of 50 ug under the test conditions. B: in-vitro human CNS cell (iNeurons or iAstrocytes) WV-40590 gymnotic uptake concentration dependent editing levels.

FIG. 28 depicts in-vivo CNS tissue (e.g., cortex, hippocampus, striatum, brain stem, cerebellum, and spinal cord) editing levels of endogenous mouse SRSF1 transcripts. Mice were dosed with control vehicle (PBS) or WV-40592 at 100 ug on day 0, or 50 ug on day 0 and 50 ug on day 2. On experimental day 6 (7 days after initial treatment exposure) CNS tissue was harvested for measurement of site-directed RNA editing in the CNS of huADAR1 p110 transgenic mice. A: average CNS editing levels induced by WV-40592 in huADAR1 p110 mice. No editing was induced by PBS. In some embodiments, single dosage using 100 ug generated a larger editing response than two temporally dispersed doses of 50 ug under the test conditions. B: in-vitro human CNS cell (iNeurons or iAstrocytes) WV-40592 gymnotic uptake concentration dependent editing levels.

FIG. 29 depicts an exemplary genetic cross of a humanized SERPINA1 mouse expressing the mutant allele huSERPINA1-Pi*Z with a huADAR mouse. The resultant offspring are double transgenic SERPINA1-Pi*Z/huADAR mice that can act as a model for in vivo editing of known mutant alleles amenable to editing by ADAR (e.g., for assessing properties and/or activities of various agents such as oligonucleotide agents).

### DEFINITIONS

The scope of the present invention is defined by the claims appended hereto and is not limited by certain embodiments described herein. Those skilled in the art, reading the present specification, will be aware of various modifications that may be equivalent to such described embodiments, or otherwise within the scope of the claims. In general, terms used herein are in accordance with their understood meaning in the art, unless clearly indicated otherwise. Explicit definitions of certain terms are provided below; meanings of these and other terms in particular instances throughout this specification will be clear to those skilled in the art from context. Additional definitions for the following and other terms are set forth throughout the specification. Patent and non-patent literature references cited within this specification, or relevant portions thereof, may be incorporated by reference where designated.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a” and “an,” as used herein, should be understood to include the plural referents unless clearly indicated to the contrary. Claims or descriptions that include “or” between one or more members of a group are considered satisfied if one, more than one, or all of the group members are present in, employed in, or otherwise relevant to a given product or process unless indicated to the contrary or otherwise evident from the context. In some embodiments, exactly one member of a group is present in, employed in, or otherwise relevant to a given product or process. In some embodiments, more than one, or all group members are present in, employed in, or otherwise relevant to a given product or process. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists (e.g., in Markush group or similar format), it is to be understood that each subgroup of the elements is also disclosed, and any element(s) can be removed from the group. It should be understood that, in general, where embodiments or aspects are referred to as “comprising” particular elements, features, etc., certain embodiments or aspects “consist,” or “consist essentially of,” such elements, features, etc. For purposes of simplicity, those embodiments have not in every case been specifically set forth in so many words herein. It should also be understood that any embodiment or aspect can be explicitly excluded from the claims, regardless of whether the specific exclusion is recited in the specification.

Unless otherwise specified, description of oligonucleotides and elements thereof (e.g., base sequence, sugar modifications, internucleotidic linkages, linkage phosphorus stereochemistry, patterns thereof, etc.) is from 5′ to 3′. As those skilled in the art will appreciate, in some embodiments, oligonucleotides may be provided and/or utilized as salt forms, particularly pharmaceutically acceptable salt forms, e.g., sodium salts. As those skilled in the art will also appreciate, in some embodiments, individual oligonucleotides within a composition may be considered to be of the same constitution and/or structure even though, within such composition (e.g., a liquid composition), particular such oligonucleotides might be in different salt form(s) (and may be dissolved and the oligonucleotide chain may exist as an anion form when, e.g., in a liquid composition) at a particular moment in time. For example, those skilled in the art will appreciate that, at a given pH, individual internucleotidic linkages along an oligonucleotide chain may be in an acid (H) form, or in one of a plurality of possible salt forms (e.g., a sodium salt, or a salt of a different cation, depending on which ions might be present in the preparation or composition), and will understand that, so long as their acid forms (e.g., replacing all cations, if any, with H⁺) are of the same constitution and/or structure, such individual oligonucleotides may properly be considered to be of the same constitution and/or structure.

Administration: as used herein, includes the administration of a composition (e.g., antigen or antibody) to a subject or system (e.g., to a cell, organ, tissue, organism, or relevant component or set of components thereof). The skilled artisan will appreciate that route of administration may vary depending, for example, on the subject or system to which the composition is being administered, the nature of the composition, the purpose of the administration, etc. For example, in certain embodiments, administration to an animal subject (e.g., to a human or a rodent) may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and/or vitreal. In some embodiments, administration may involve intermittent dosing. In some embodiments, administration may involve continuous dosing (e.g., perfusion) for at least a selected period of time.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, a non-human animal is a mammal (e.g., a rodent, a mouse, a rat, a rabbit, a monkey, a dog, a cat, a sheep, cattle, a primate and/or a pig). In some embodiments, a non-human animal is a non-primate. In some embodiments, a non-human animal is a rodent. In some embodiments, a non-human animal is a rat. In some embodiments, a non-human animal is a mouse. In some embodiments, animals include, but are not limited to, mammals, birds, reptiles, amphibians, fish and/or worms. In some embodiments, an animal may be a transgenic animal, a genetically-engineered animal and/or a clone.

Approximately: as applied to one or more values of interest, includes to a value that is similar to a stated reference value. In certain embodiments, the term “approximately” or “about” refers to a range of values that fall within ± 10% (greater than or less than) of the stated reference value unless otherwise stated or otherwise evident from the context (except where such number would exceed 100% of a possible value).

Biologically active: as used herein, refers to a characteristic of any agent that has activity in a biological system, in vitro or in vivo (e.g., in an organism). For instance, an agent that, when present in an organism, has a biological effect within that organism is considered to be biologically active. In particular embodiments, where a protein or polypeptide is biologically active, a portion of that protein or polypeptide that shares at least one biological activity of the protein or polypeptide is typically referred to as a “biologically active” portion.

Characteristic portion: As used herein, the term “characteristic portion”, in the broadest sense, refers to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity. In certain embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to the sequence and/or structural identity specified above, shares at least one functional characteristic with the relevant intact substance. In some embodiments, a characteristic portion may be biologically active.

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of the polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share the sequence element.

Chirally controlled oligonucleotide composition: The terms “chirally controlled oligonucleotide composition”, “chirally controlled nucleic acid composition”, and the like, as used herein, refers to a composition that comprises a plurality of oligonucleotides (or nucleic acids) which share a common base sequence, wherein the plurality of oligonucleotides (or nucleic acids) share the same linkage phosphorus stereochemistry at one or more chiral internucleotidic linkages (chirally controlled or stereodefined internucleotidic linkages, whose chiral linkage phosphorus is Rp or Sp in the composition (“stereodefined”), not a random Rp and Sp mixture as non-chirally controlled internucleotidic linkages). In some embodiments, a chirally controlled oligonucleotide composition comprises a plurality of oligonucleotides (or nucleic acids) that share: 1) a common base sequence, 2) a common pattern of backbone linkages, and 3) a common pattern of backbone phosphorus modifications, wherein the plurality of oligonucleotides (or nucleic acids) share the same linkage phosphorus stereochemistry at one or more chiral internucleotidic linkages (chirally controlled or stereodefined internucleotidic linkages, whose chiral linkage phosphorus is Rp or Sp in the composition (“stereodefined”), not a random Rp and Sp mixture as non-chirally controlled internucleotidic linkages). Level of the plurality of oligonucleotides (or nucleic acids) in a chirally controlled oligonucleotide composition is pre-determined/controlled or enriched (e.g., through chirally controlled oligonucleotide preparation to stereoselectively form one or more chiral internucleotidic linkages) compared to a random level in a non-chirally controlled oligonucleotide composition. In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition are oligonucleotides of the plurality. In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition that share the common base sequence, the common pattern of backbone linkages, and the common pattern of backbone phosphorus modifications are oligonucleotides of the plurality. In some embodiments, a level is about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a composition, or of all oligonucleotides in a composition that share a common base sequence (e.g., of a plurality of oligonucleotide or an oligonucleotide type), or of all oligonucleotides in a composition that share a common base sequence, a common pattern of backbone linkages, and a common pattern of backbone phosphorus modifications, or of all oligonucleotides in a composition that share a common base sequence, a common patter of base modifications, a common pattern of sugar modifications, a common pattern of internucleotidic linkage types, and/or a common pattern of internucleotidic linkage modifications. In some embodiments, the plurality of oligonucleotides share the same stereochemistry at about 1-50 (e.g., about 1-10, 1-20, 5-10, 5-20, 10-15, 10-20, 10-25, 10-30, or about 1, 2, 3, 4, 5, 6, 7, 8, 9, 10, 11, 12, 13, 14, 15, 16, 17, 18, 19, or 20, or at least 1, 2, 3, 4, 5, 6, 7, 8, 9, 10, 11, 12, 13, 14, 15, 16, 17, 18, 19, or 20) chiral internucleotidic linkages. In some embodiments, the plurality of oligonucleotides share the same stereochemistry at about 1%-100% (e.g., about 5%-100%, 10%-100%, 20%-100%, 30%-100%, 40%-100%, 50%-100%, 60%-100%, 70%-100%, 80-100%, 90-100%, 95-100%, 50%-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 99%) of chiral internucleotidic linkages. In some embodiments, oligonucleotides (or nucleic acids) of a plurality share the same pattern of sugar and/or nucleobase modifications, in any. In some embodiments, oligonucleotides (or nucleic acids) of a plurality are various forms of the same oligonucleotide (e.g., acid and/or various salts of the same oligonucleotide). In some embodiments, oligonucleotides (or nucleic acids) of a plurality are of the same constitution. In some embodiments, level of the oligonucleotides (or nucleic acids) of the plurality is about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides (or nucleic acids) in a composition that share the same constitution as the oligonucleotides (or nucleic acids) of the plurality. In some embodiments, each chiral internucleotidic linkage is a chiral controlled internucleotidic linkage, and the composition is a completely chirally controlled oligonucleotide composition. In some embodiments, oligonucleotides (or nucleic acids) of a plurality are structurally identical. In some embodiments, a chirally controlled internucleotidic linkage has a diastereopurity of at least 80%, 85%, 90%, has a diastereopurity of at least 96%. In some embodiments, a chirally controlled internucleotidic linkage has a diastereopurity of at least 97%. In some embodiments, a chirally controlled internucleotidic linkage has a diastereopurity of at least 98%. In some embodiments, a chirally controlled internucleotidic linkage has a diastereopurity of at least 99%. In some embodiments, a percentage of a level is or is at least (DS)^{nc}, wherein DS is a diastereopurity as described in the present disclosure (e.g., 90%, 91%, 92%, 93%, 94%, 95%, 96%, 97%, 98%, 99% or 99.5% or more) and nc is the number of chirally controlled internucleotidic linkages as described in the present disclosure (e.g., 1-50, 1-40, 1-30, 1-25, 1-20, 5-50, 5-40, 5-30, 5-25, 5- 20, 1, 2, 3, 4, 5, 6, 7, 8, 9, 10, 11, 12, 13, 14, 15, 16, 17, 18, 19, 20, 21, 22, 23, 24, 25 or more). In some embodiments, a percentage of a level is or is at least (DS)^{nc}, wherein DS is 95%-100%. For example, when DS is 99% and nc is 10, the percentage is or is at least 90% ((99%)¹⁰ ≈ 0.90 = 90%). In some embodiments, level of a plurality of oligonucleotides in a composition is represented as the product of the diastereopurity of each chirally controlled internucleotidic linkage in the oligonucleotides. In some embodiments, diastereopurity of an internucleotidic linkage connecting two nucleosides in an oligonucleotide (or nucleic acid) is represented by the diastereopurity of an internucleotidic linkage of a dimer connecting the same two nucleosides, wherein the dimer is prepared using comparable conditions, in some instances, identical synthetic cycle conditions (e.g., for the linkage between Nx and Ny in an oligonucleotide ....NxNy....., the dimer is NxNy). In some embodiments, not all chiral internucleotidic linkages are chiral controlled internucleotidic linkages, and the composition is a partially chirally controlled oligonucleotide composition. In some embodiments, a non-chirally controlled internucleotidic linkage has a diastereopurity of less than about 80%, 75%, 70%, 65%, 60%, 55%, or of about 50%, as typically observed in stereorandom oligonucleotide compositions (e.g., as appreciated by those skilled in the art, from traditional oligonucleotide synthesis, e.g., the phosphoramidite method). In some embodiments, oligonucleotides (or nucleic acids) of a plurality are of the same type. In some embodiments, a chirally controlled oligonucleotide composition comprises non-random or controlled levels of individual oligonucleotide or nucleic acids types. For instance, in some embodiments a chirally controlled oligonucleotide composition comprises one and no more than one oligonucleotide type. In some embodiments, a chirally controlled oligonucleotide composition comprises more than one oligonucleotide type. In some embodiments, a chirally controlled oligonucleotide composition comprises multiple oligonucleotide types. In some embodiments, a chirally controlled oligonucleotide composition is a composition of oligonucleotides of an oligonucleotide type, which composition comprises a non-random or controlled level of a plurality of oligonucleotides of the oligonucleotide type. 91%, 92%, 93%, 94%, 95%, 96%, 97%, 98%, 99% or 99.5%, typically at least 90%, 91%, 92%, 93%, 94%, 95%, 96%, 97%, 98%, 99% or 99.5%. In some embodiments, a chirally controlled internucleotidic linkage has a diastereopurity of at least 95%. In some embodiments, a chirally controlled internucleotidic linkage. In some embodiments, an oligonucleotide composition in the present disclosure is a chirally controlled oligonucleotide composition.

Comparable: The term “comparable” is used herein to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Conservative: as used herein, refers to instances when describing a conservative amino acid substitution, including a substitution of an amino acid residue by another amino acid residue having a side chain R group with similar chemical properties (e.g., charge or hydrophobicity). In general, a conservative amino acid substitution will not substantially change the functional properties of interest of a protein, for example, the ability of a receptor to bind to a ligand. Examples of groups of amino acids that have side chains with similar chemical properties include: aliphatic side chains such as glycine (Gly, G), alanine (Ala, A), valine (Val, V), leucine (Leu, L), and isoleucine (Ile, I); aliphatic-hydroxyl side chains such as serine (Ser, S) and threonine (Thr, T); amide-containing side chains such as asparagine (Asn, N) and glutamine (Gln, Q); aromatic side chains such as phenylalanine (Phe, F), tyrosine (Tyr, Y), and tryptophan (Trp, W); basic side chains such as lysine (Lys, K), arginine (Arg, R), and histidine (His, H); acidic side chains such as aspartic acid (Asp, D) and glutamic acid (Glu, E); and sulfur-containing side chains such as cysteine (Cys, C) and methionine (Met, M). Conservative amino acids substitution groups include, for example, valine/leucine/isoleucine (Val/Leu/Ile, V/L/I), phenylalanine/tyrosine (Phe/Tyr, F/Y), lysine/arginine (Lys/Arg, K/R), alanine/valine (Ala/Val, A/V), glutamate/aspartate (Glu/Asp, E/D), and asparagine/glutamine (Asn/Gln, N/Q). In some embodiments, a conservative amino acid substitution can be a substitution of any native residue in a protein with alanine, as used in, for example, alanine scanning mutagenesis. In some embodiments, a conservative substitution is made that has a positive value in the PAM250 log-likelihood matrix disclosed in Gonnet, G.H. et al., 1992, Science 256:1443-1445. In some embodiments, a substitution is a moderately conservative substitution wherein the substitution has a nonnegative value in the PAM250 log-likelihood matrix.

Control: as used herein, refers to the art-understood meaning of a “control” being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. A “control” also includes a “control animal.” A “control animal” may have a modification as described herein, a modification that is different as described herein, or no modification (i.e., a wild-type animal). In one experiment, a “test” parameter (e.g., a variable being tested) is applied. In a second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control.

Disruption: as used herein, refers to the result of a homologous recombination event with a DNA molecule (e.g., with an endogenous homologous sequence such as a gene or gene locus). In some embodiments, a disruption may achieve or represent an insertion, deletion, substitution, replacement, missense mutation, or a frame-shift of a DNA sequence(s), or any combination thereof. Insertions may include the insertion of entire genes or gene fragments, e.g., exons, which may be of an origin other than the endogenous sequence (e.g., a heterologous sequence). In some embodiments, a disruption may increase expression and/or activity of a gene or gene product (e.g., of a polypeptide encoded by a gene). In some embodiments, a disruption may decrease expression and/or activity of a gene or gene product. In some embodiments, a disruption may alter sequence of a gene or an encoded gene product (e.g., an encoded polypeptide). In some embodiments, a disruption may truncate or fragment a gene or an encoded gene product (e.g., an encoded polypeptide). In some embodiments, a disruption may extend a gene or an encoded gene product. In some such embodiments, a disruption may achieve assembly of a fusion polypeptide. In some embodiments, a disruption may affect level, but not activity, of a gene or gene product. In some embodiments, a disruption may affect activity, but not level, of a gene or gene product. In some embodiments, a disruption may have no significant effect on level of a gene or gene product. In some embodiments, a disruption may have no significant effect on activity of a gene or gene product. In some embodiments, a disruption may have no significant effect on either level or activity of a gene or gene product.

Endogenous promoter: as used herein, refers to a promoter that is naturally associated, e.g., in a wild-type organism, with an endogenous gene.

Engineered: as used herein refers, in general, to the aspect of having been manipulated by the hand of man. For example, in some embodiments, a polynucleotide may be considered to be “engineered” when two or more sequences that are not linked together in that order in nature are manipulated by the hand of man to be directly linked to one another in the engineered polynucleotide. In some embodiments, an engineered polynucleotide may comprise a regulatory sequence that is found in nature in operative association with a first coding sequence but not in operative association with a second coding sequence, is linked by the hand of man so that it is operatively associated with the second coding sequence. Alternatively, or additionally, in some embodiments, first and second nucleic acid sequences that each encode polypeptide elements or domains that in nature are not linked to one another may be linked to one another in a single engineered polynucleotide. Comparably, in some embodiments, a cell or organism may be considered to be “engineered” if it has been manipulated so that its genetic information is altered (e.g., new genetic material not previously present has been introduced, or previously present genetic material has been altered or removed). As is common practice and is understood by persons of skill in the art, progeny of an engineered polynucleotide or cell are typically still referred to as “engineered” even though the actual manipulation was performed on a prior entity. Furthermore, as will be appreciated by persons of skill in the art, a variety of methodologies are available through which “engineering” as described herein may be achieved. For example, in some embodiments, “engineering” may involve selection or design (e.g., of nucleic acid sequences, polypeptide sequences, cells, tissues, and/or organisms) through use of computer systems programmed to perform analysis or comparison, or otherwise to analyze, recommend, and/or select sequences, alterations, etc.). Alternatively, or additionally, in some embodiments, “engineering” may involve use of in vitro chemical synthesis methodologies and/or recombinant nucleic acid technologies such as, for example, nucleic acid amplification (e.g., via the polymerase chain reaction) hybridization, mutation, transformation, transfection, etc., and/or any of a variety of controlled mating methodologies. As will be appreciated by those skilled in the art, a variety of established such techniques (e.g., for recombinant DNA, oligonucleotide synthesis, and tissue culture and transformation (e.g., electroporation, lipofection, etc.)) are well known in the art and described in various general and more specific references that are cited and/or discussed throughout the present specification. See e.g., Sambrook et al., Molecular Cloning: A Laboratory Manual 2nd ed., Cold Spring Harbor Laboratory Press, Cold Spring Harbor, N.Y., 1989 and Principles of Gene Manipulation: An Introduction to Genetic Manipulation, 5th Ed., ed. By Old, R.W. and S.B. Primrose, Blackwell Science, Inc., 1994.

Gene: as used herein, refers to a DNA sequence in a chromosome that codes for a product (e.g., an RNA product and/or a polypeptide product). In some embodiments, a gene includes coding sequence (i.e., sequence that encodes a particular product). In some embodiments, a gene includes non-coding sequence. In some particular embodiments, a gene may include both coding (e.g., exonic) and non-coding (e.g., intronic) sequence. In some embodiments, a gene may include one or more regulatory sequences (e.g., promoters, enhancers, etc.) and/or intron sequences that, for example, may control or impact one or more aspects of gene expression (e.g., cell-type-specific expression, inducible expression, etc.). For the purpose of clarity, we note that, as used in the present disclosure, the term “gene” generally refers to a portion of a nucleic acid that encodes a polypeptide or fragment thereof; the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a polypeptide-coding nucleic acid.

Genetically modified non-human animal or genetically engineered non-human animal: are used interchangeably herein and refer to any non-naturally occurring non-human animal (e.g., a rodent, e.g., a rat or a mouse) in which one or more of the cells of the non-human animal contain heterologous nucleic acid and/or gene encoding a polypeptide of interest, in whole or in part. For example, in some embodiments, a “genetically modified non-human animal” or “genetically engineered non-human animal” refers to non-human animal that contains a transgene or transgene construct as described herein. In some embodiments, a heterologous nucleic acid and/or gene is introduced into the cell, directly or indirectly by introduction into a precursor cell, by way of deliberate genetic manipulation, such as by microinjection or by infection with a recombinant virus. The term genetic manipulation does not include classic breeding techniques, but rather is directed to introduction of recombinant DNA molecule(s). This molecule may be integrated within a chromosome. The phrases “genetically modified non-human animal” or “genetically engineered non-human animal” refers to animals that are heterozygous or homozygous for a heterologous nucleic acid and/or gene, and/or animals that have single or multi-copies of a heterologous nucleic acid and/or gene.

Germline Genome: as used herein, refers to the genome found in a germ cell (e.g., a gamete, e.g., a sperm or egg) used in the formation of an animal. A germline genome is a source of genomic DNA for cells in an animal. In some embodiments, an animal (e.g., a mouse or rat) having a modification in its germline genome is considered to have the modification in the genomic DNA of all of its cells.

Germline Sequence: as used herein, refers to a DNA sequence as found in an endogenous germline genome of a wild-type animal (e.g., mouse, rat, or human), or an RNA or amino acid sequence encoded by a DNA sequence as found in an endogenous germline genome of an animal (e.g., mouse, rat, or human).

Heterologous: as used herein, refers to an agent or entity from a different source. For example, when used in reference to a polypeptide, gene, or gene product present in a particular cell or organism, the term clarifies that the relevant polypeptide, gene, or gene product: 1) was engineered by the hand of man; 2) was introduced into the cell or organism (or a precursor thereof) through the hand of man (e.g., via genetic engineering); and/or 3) is not naturally produced by or present in the relevant cell or organism (e.g., the relevant cell type or organism type). “Heterologous” also includes a polypeptide, gene or gene product that is normally present in a particular native cell or organism, but has been altered or modified, for example, by mutation or placement under the control of non-naturally associated and, in some embodiments, non-endogenous regulatory elements (e.g., a promoter).

Host cell: as used herein, refers to a cell into which a nucleic acid or protein has been introduced. Persons of skill upon reading this disclosure will understand that such a term refers not only to the particular subject cell, but also is used to refer to the progeny of such a cell. Because certain modifications may occur in succeeding generations due to either mutation or environmental influences, such progeny may not, in fact, be identical to the parent cell, but are still included within the scope of the phrase “host cell.” In some embodiments, a host cell is or comprises a prokaryotic or eukaryotic cell. In general, a host cell is any cell that is suitable for receiving and/or producing a heterologous nucleic acid or protein, regardless of the Kingdom of life to which the cell is designated. Exemplary cells include those of prokaryotes and eukaryotes (single-cell or multiple-cell), bacterial cells (e.g., strains of Escherichia coli, Bacillus spp., Streptomyces spp., etc.), mycobacteria cells, fungal cells, yeast cells (e.g., Saccharomyces cerevisiae, Schizosaccharomyces pombe, Pichia pastoris, Pichia methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, a cell is a human, monkey, ape, hamster, rat, or mouse cell. In some embodiments, a cell is eukaryotic and is selected from the following cells: Chinese Hamster Ovarian (CHO) (e.g., CHO K1, DXB-11 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3A cell, HT1080 cell, myeloma cell, tumor cell, and a cell line derived from an aforementioned cell. In some embodiments, a cell comprises one or more viral genes, e.g., a retinal cell that expresses a viral gene (e.g., a PER.C6® cell). In some embodiments, a host cell is or comprises an isolated cell. In some embodiments, a host cell is part of a tissue. In some embodiments, a host cell is part of an organism.

Identity: As used herein, the term “identity” refers to the overall relatedness between polymeric molecules, e.g., between nucleic acid molecules (e.g., oligonucleotides, DNA, RNA, etc.) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix. In certain embodiments, Identity as used herein in connection with a comparison of sequences, refers to identity as determined by a number of different algorithms known in the art that can be used to measure nucleotide and/or amino acid sequence identity. In some embodiments, identities as described herein are determined using a ClustalW v. 1.83 (slow) alignment employing an open gap penalty of 10.0, an extend gap penalty of 0.1, and using a Gonnet similarity matrix (MACVECTOR™ 10.0.2, MacVector Inc., 2008).

In place of: as used herein, refers to a positional substitution in which a first nucleic acid sequence is located at the position of a second nucleic acid sequence in a chromosome (e.g., where the second nucleic acid sequence was previously (e.g., originally) located in a chromosome, e.g., at the endogenous locus of the second nucleic acid sequence). The phrase “in place of” does not require that the second nucleic acid sequence be removed from, e.g., a locus or chromosome. In some embodiments, the second nucleic acid sequence and the first nucleic acid sequence are comparable to one another in that, for example, the first and second sequences are homologous to one another, contain corresponding elements (e.g., protein-coding elements, regulatory elements, etc.), and/or have similar or identical sequences. In some embodiments, a first and/or second nucleic acid sequence includes one or more of a promoter, an enhancer, a splice donor site, a splice acceptor site, an intron, an exon, an untranslated region (UTR); in some embodiments, a first and/or second nucleic acid sequence includes one or more coding sequences. In some embodiments, a first nucleic acid sequence is a homolog or variant (e.g., mutant) of the second nucleic acid sequence. In some embodiments, a first nucleic acid sequence is an ortholog or homolog of the second sequence. In some embodiments, a first nucleic acid sequence is or comprises a human nucleic acid sequence. In some embodiments, including where the first nucleic acid sequence is or comprises a human nucleic acid sequence, the second nucleic acid sequence is or comprises a rodent sequence (e.g., a mouse or rat sequence). In some embodiments, including where the first nucleic acid sequence is or comprises a human nucleic acid sequence, the second nucleic acid sequence is or comprises a human sequence. In some embodiments, a first nucleic acid sequence is a variant or mutant (i.e., a sequence that contains one or more sequence differences, e.g., substitutions, as compared to the second sequence) of the second sequence. The nucleic acid sequence so placed may include one or more regulatory sequences that are part of source nucleic acid sequence used to obtain the sequence so placed (e.g., promoters, enhancers, 5′- or 3′-untranslated regions, etc.). For example, in various embodiments, a first nucleic acid sequence is a substitution of an endogenous sequence with a heterologous sequence that results in the production of a gene product from the nucleic acid sequence so placed (comprising the heterologous sequence), but not expression of the endogenous sequence; a first nucleic acid sequence is of an endogenous genomic sequence with a nucleic acid sequence that encodes a polypeptide that has a similar function as a polypeptide encoded by the endogenous sequence (e.g., the endogenous genomic sequence encodes a non-human variable region polypeptide, in whole or in part, and the DNA fragment encodes one or more human variable region polypeptides, in whole or in part). In various embodiments, a human or non-human primate ADAR gene segment or fragment thereof is in place of an endogenous non-human animal (e.g., rodent, e.g., rat or mouse) gene segment or fragment.

In vitro: as used herein refers to events that occur in an artificial environment, e.g., in a test tube or reaction vessel, in cell culture, etc., rather than within a multi-cellular organism.

In vivo: as used herein refers to events that occur within a multi-cellular organism, such as a human and/or a non-human animal. In the context of cell-based systems, the term may be used to refer to events that occur within a living cell (as opposed to, for example, in vitro systems).

Isolated: as used herein, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are separated from 10% to 100%, 15%-100%, 20%-100%, 25%-100%, 30%-100%, 35%-100%, 40%-100%, 45%-100%, 50%-100%, 55%-100%, 60%-100%, 65%-100%, 70%-100%, 75%-100%, 80%-100%, 85%-100%, 90%-100%, 95%-100%, 96%-100%, 97%-100%, 98%-100%, or 99%-100% of the other components with which they were initially associated. In some embodiments, isolated agents are separated from 10% to 100%, 10%-99%, 10%-98%, 10%-97%, 10%-96%, 10%-95%, 10%-90%, 10%-85%, 10%-80%, 10%-75%, 10%-70%, 10%-65%, 10%-60%, 10%-55%, 10%-50%, 10%-45%, 10%-40%, 10%-35%, 10%-30%, 10%-25%, 10%-20%, or 10%-15% of the other components with which they were initially associated. In some embodiments, isolated agents are separated from 11% to 99%, 12%-98%, 13%-97%, 14%-96%, 15%-95%, 20%-90%, 25%-85%, 30%-80%, 35%-75%, 40%-70%, 45%-65%, 50%-60%, or 55%-60% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. In some embodiments, isolated agents are 80%-99%, 85%-99%, 90%-99%, 95%-99%, 96%-99%, 97%-99%, or 98%-99% pure. In some embodiments, isolated agents are 80%-99%, 80%-98%, 80%-97%, 80%-96%, 80%-95%, 80%-90%, or 80%-85% pure. In some embodiments, isolated agents are 85%-98%, 90%-97%, or 95%-96% pure. In some embodiments, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when: a) by virtue of its origin or source of derivation is not associated with some or all of the components that accompany it in its native state in nature; b) it is substantially free of other polypeptides or nucleic acids of the same species from the species that produces it in nature; or c) is expressed by or is otherwise in association with components from a cell or other expression system that is not of the species that produces it in nature. Thus, for instance, in some embodiments, a polypeptide that is chemically synthesized, or is synthesized in a cellular system different from that which produces it in nature, is considered to be an “isolated” polypeptide. Alternatively, or additionally, in some embodiments, a polypeptide that has been subjected to one or more purification techniques may be considered to be an “isolated” polypeptide to the extent that it has been separated from other components: a) with which it is associated in nature; and/or b) with which it was associated when initially produced.

Naturally appears: as used herein in reference to a biological element (e.g., a nucleic acid sequence) means that the biological element can be found in a specified context and/or location, absent engineering (e.g., genetic engineering), in a cell or organism (e.g., an animal). In other words, a sequence that naturally appears in a specified context and/or location is not in the specified context and/or location as the result of engineering (e.g., genetic engineering).

Non-human animal: as used herein, refers to any vertebrate organism that is not a human. In some embodiments, a non-human animal is a cyclostome, a bony fish, a cartilaginous fish (e.g., a shark or a ray), an amphibian, a reptile, a mammal, and a bird. In some embodiments, a non-human animal is a mammal. In some embodiments, a non-human mammal is a primate, a goat, a sheep, a pig, a dog, a cow, or a rodent. In some embodiments, a non-human animal is a rodent such as a rat or a mouse. In some embodiments, a non-human animal is a rat. In some embodiments, a non-human animal is a mouse.

Operably linked: as used herein, refers to a juxtaposition of components, where the components described are in a relationship permitting them to function in their intended manner (e.g., when the components are present in the proper tissue, cell type, cellular activity, etc.). A control sequence “operably linked” to a coding sequence is ligated in such a way that expression of the coding sequence is achieved under conditions compatible with the control sequences. “Operably linked” sequences include both expression control sequences that are contiguous with a gene of interest and expression control sequences that act in trans or at a distance to control a gene of interest (or sequence of interest). The term “expression control sequence” includes polynucleotide sequences, which are necessary to affect the expression and processing of coding sequences to which they are ligated. “Expression control sequences” include: appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance polypeptide stability; and when desired, sequences that enhance polypeptide secretion. The nature of such control sequences differs depending upon the host organism. For example, in prokaryotes, such control sequences generally include promoter, ribosomal binding site and transcription termination sequence, while in eukaryotes typically such control sequences include promoters and transcription termination sequence. The term “control sequences” is intended to include components whose presence is essential for expression and processing, and can also include additional components whose presence is advantageous, for example, leader sequences and fusion partner sequences.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer’s solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups, e.g., an oligonucleotide, and a pharmaceutically acceptable salt is an alkali, alkaline earth metal, or ammonium (e.g., an ammonium salt of N(R)3, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups, for example, an oligonucleotide may comprise two or more acidic groups (e.g., in natural phosphate linkages and/or modified internucleotidic linkages). In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations. In some embodiments, each phosphorothioate and phosphate group independently exists in its salt form (e.g., if sodium salt, —O—P(O)(SNa)—O— and —O—P(O)(ONa)—O—, respectively). In some embodiments, each phosphorothioate and phosphate internucleotidic linkage independently exists in its salt form (e.g., if sodium salt, —O—P(O)(SNa)—O— and —O—P(O)(ONa)—O—, respectively). In some embodiments, a pharmaceutically acceptable salt is a sodium salt of an oligonucleotide. In some embodiments, a pharmaceutically acceptable salt is a sodium salt of an oligonucleotide, wherein each acidic phosphate and modified phosphate group (e.g., phosphorothioate, phosphate, etc.), if any, exists as a salt form (all sodium salt).

Polypeptide: As used herein refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide has an amino acid sequence encoded by a sequence that does not occur in nature (e.g., a sequence that is engineered in that it is designed and/or produced through action of the hand of man to encode said polypeptide). In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide’s N-terminus, at the polypeptide’s C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Recombinant: as used herein, is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc) that is transgenic for or otherwise has been manipulated to express a gene or genes, or gene components that encode and/or direct expression of the polypeptide or one or more component(s), portion(s), element(s), or domain(s) thereof, and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc). In some embodiments, a recombinant polypeptide has an amino acid sequence that resulted from mutagenesis (e.g., in vitro or in vivo, for example, in a non-human animal), so that the amino acid sequences of the recombinant polypeptides are sequences that, while originating from and related to polypeptides sequences, may not naturally exist within the genome of a non-human animal in vivo.

Reference: as used herein, refers to a standard or control agent, animal, cohort, individual, population, sample, sequence or value against which an agent, animal, cohort, individual, population, sample, sequence or value of interest is compared. In some embodiments, a reference agent, animal, cohort, individual, population, sample, sequence or value is tested and/or determined substantially simultaneously with the testing or determination of an agent, animal, cohort, individual, population, sample, sequence or value of interest. In some embodiments, a reference agent, animal, cohort, individual, population, sample, sequence or value is a historical reference, optionally embodied in a tangible medium. In some embodiments, a reference may refer to a control. A “reference” also includes a “reference animal.” A “reference animal” may have a modification as described herein, a modification that is different as described herein or no modification (i.e., a wild-type animal). Typically, as would be understood by persons of skill in the art, a reference agent, animal, cohort, individual, population, sample, sequence or value is determined or characterized under conditions comparable to those utilized to determine or characterize an agent, animal (e.g., a mammal), cohort, individual, population, sample, sequence or value of interest.

Replacement: as used herein, refers to a process through which a “replaced” nucleic acid sequence (e.g., a gene) found in a host locus (e.g., in a genome) is removed from that locus, and a different, “replacement” nucleic acid is located in its place. In some embodiments, the replaced nucleic acid sequence and the replacement nucleic acid sequences are comparable to one another in that, for example, they are homologous to one another, contain corresponding elements (e.g., protein-coding elements, regulatory elements, etc.), and/or have similar or identical sequences. In some embodiments, a replaced nucleic acid sequence includes one or more of a promoter, an enhancer, a splice donor site, a splice acceptor site, an intron, an exon, an untranslated region (UTR); in some embodiments, a replacement nucleic acid sequence includes one or more coding sequences. In some embodiments, a replacement nucleic acid sequence is a homolog or variant (e.g., mutant) of the replaced nucleic acid sequence. In some embodiments, a replacement nucleic acid sequence is an ortholog or homolog of the replaced sequence. In some embodiments, a replacement nucleic acid sequence is or comprises a human nucleic acid sequence. In some embodiments, including where the replacement nucleic acid sequence is or comprises a human nucleic acid sequence, the replaced nucleic acid sequence is or comprises a rodent sequence (e.g., a mouse or rat sequence). In some embodiments, including where the replacement nucleic acid sequence is or comprises a human nucleic acid sequence, the replaced nucleic acid sequence is or comprises a human sequence. In some embodiments, a replacement nucleic acid sequence is a variant or mutant (i.e., a sequence that contains one or more sequence differences, e.g., substitutions, as compared to the replaced sequence) of the replaced sequence. The nucleic acid sequence so placed may include one or more regulatory sequences that are part of source nucleic acid sequence used to obtain the sequence so placed (e.g., promoters, enhancers, 5′- or 3′-untranslated regions, etc.). For example, in various embodiments, a replacement is a substitution of an endogenous sequence with a heterologous sequence that results in the production of a gene product from the nucleic acid sequence so placed (comprising the heterologous sequence), but not expression of the endogenous sequence; a replacement is of an endogenous genomic sequence with a nucleic acid sequence that encodes a polypeptide that has a similar function as a polypeptide encoded by the endogenous sequence. In some embodiments, an endogenous non-human ADAR1 gene segment or fragment thereof is replaced with a human ADAR1 gene segment or fragment thereof.

Subject: As used herein, the term “subject” or “test subject” refers to any organism to which a compound (e.g., an oligonucleotide) or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject is a human. In some embodiments, a subject may be suffering from and/or susceptible to a disease, disorder and/or condition.

Substantially: as used herein, refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena. A base sequence which is substantially identical or complementary to a second sequence is not fully identical or complementary to the second sequence, but is mostly or nearly identical or complementary to the second sequence. In some embodiments, an oligonucleotide with a substantially complementary sequence to another oligonucleotide or nucleic acid forms duplex with the oligonucleotide or nucleic acid in a similar fashion as an oligonucleotide with a fully complementary sequence.

Substantial similarity: as used herein, refers to a comparison between amino acid or nucleic acid sequences. As will be appreciated by those of ordinary skill in the art, two sequences are generally considered to be “substantially similar” if they contain similar residues (e.g., amino acids or nucleotides) in corresponding positions. As is understood in the art, while similar residues may be identical residues (see also Substantial Identity, below), similar residues may also be non-identical residues with appropriately comparable structural and/or functional characteristics. For example, as is well known by those of ordinary skill in the art, certain amino acids are typically classified as “hydrophobic” or “hydrophilic” amino acids, and/or as having “polar” or “non-polar” side chains. Substitution of one amino acid for another of the same type may often be considered a “conservative” substitution. Typical amino acid categorizations are summarized in the table below.

| | | | | | |
|---|---|---|---|---|---|
| Alanine | Ala | A | Nonpolar | Neutral | 1.8 |
| Arginine | Arg | R | Polar | Positive | -4.5 |
| Asparagine | Asn | N | Polar | Neutral | -3.5 |
| Aspartic acid | Asp | D | Polar | Negative | -3.5 |
| Cysteine | Cys | C | Nonpolar | Neutral | 2.5 |
| Glutamic acid | Glu | E | Polar | Negative | -3.5 |
| Glutamine | Gln | Q | Polar | Neutral | -3.5 |
| Glycine | Gly | G | Nonpolar | Neutral | -0.4 |
| Histidine | His | H | Polar | Positive | -3.2 |
| Isoleucine | Ile | I | Nonpolar | Neutral | 4.5 |
| Leucine | Leu | L | Nonpolar | Neutral | 3.8 |

| | | | | | |
|---|---|---|---|---|---|
| Lysine | Lys | K | Polar | Positive | -3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | -1.6 |
| Serine | Ser | S | Polar | Neutral | -0.8 |
| Threonine | Thr | T | Polar | Neutral | -0.7 |
| Tryptophan | Trp | W | Nonpolar | Neutral | -0.9 |
| Tyrosine | Tyr | Y | Polar | Neutral | -1.3 |
| Valine | Val | V | Nonpolar | Neutral | 4.2 |

| | | |
|---|---|---|
| Ambiguous Amino Acids | 3-Letter | 1-Letter |
| Asparagine or aspartic acid | Asx | B |
| Glutamine or glutamic acid | Glx | Z |
| Leucine or Isoleucine | Xle | J |
| Unspecified or unknown amino acid | Xaa | X |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, S. F. et al., 1990, J. Mol. Biol., 215(3): 403-10; Altschul, S.F. et al., 1996, Meth. Enzymol. 266:460-80; Altschul, S.F. et al., 1997, Nucleic Acids Res., 25:3389-402; Baxevanis, A.D. and B.F.F. Ouellette (eds.) Bioinformatics: A Practical Guide to the Analysis of Genes and Proteins, Wiley, 1998; and Misener et al. (eds.) Bioinformatics Methods and Protocols, Methods in Molecular Biology, Vol. 132, Humana Press, 1998. In addition to identifying similar sequences, the programs mentioned above typically provide an indication of the degree of similarity. In some embodiments, two sequences are considered to be substantially similar if at least, e.g., but not limited to, 80%, 85%, 90%, 91%, 92%, 93%, 94%, 95%, 96%, 97%, 98%, 99% or more of their corresponding residues are similar (e.g., identical or include a conservative substitution) over a relevant stretch of residues. In some embodiments, the relevant stretch is a complete sequence (e.g. a sequence of a gene, a gene segment, a sequence encoding a domain, a polypeptide, or a domain). In some embodiments, the relevant stretch is at least 9, 10, 11, 12, 13, 14, 15, 16, 17 or more residues. In some embodiments, the relevant stretch is at least 10, 15, 20, 25, 30, 35, 40, 45, 50, or more residues. In some embodiments, the relevant stretch includes contiguous residues along a complete sequence. In some embodiments, the relevant stretch includes discontinuous residues along a complete sequence, for example, noncontiguous residues brought together by the folded conformation of a polypeptide or a portion thereof.

Substantial identity: as used herein, refers to a comparison between amino acid or nucleic acid sequences. As will be appreciated by those of ordinary skill in the art, two sequences are generally considered to be “substantially identical” if they contain identical residues (e.g., amino acids or nucleotides) in corresponding positions. As is well-known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, S. F. et al., 1990, J. Mol. Biol., 215(3): 403-10; Altschul, S.F. et al., 1996, Meth. Enzymol. 266:460-80; Altschul, S.F. et al., 1997, Nucleic Acids Res., 25:3389-402; Baxevanis, A.D. and B.F.F. Ouellette (eds.) Bioinformatics: A Practical Guide to the Analysis of Genes and Proteins, Wiley, 1998; and Misener et al. (eds.) Bioinformatics Methods and Protocols, Methods in Molecular Biology, Vol. 132, Humana Press, 1998. In addition to identifying identical sequences, the programs mentioned above typically provide an indication of the degree of identity. In some embodiments, two sequences are considered to be substantially identical if at least 80%, 85%, 90%, 91%, 92%, 93%, 94%, 95%, 96%, 97%, 98%, 99% or more of their corresponding residues are identical over a relevant stretch of residues. In some embodiments, a relevant stretch of residues is a complete sequence. In some embodiments, a relevant stretch of residues is, e.g., but not limited to, at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Targeting construct or targeting vector: as used herein, refers to a polynucleotide molecule that comprises a targeting region. A targeting region comprises a sequence that is identical or substantially identical to a sequence in a target cell, tissue or animal and provides for integration of the targeting construct into a position within the genome of the cell, tissue or animal via homologous recombination. Targeting regions that target using site-specific recombinase recognition sites (e.g., loxP or Frt sites) are also included and described herein. In some embodiments, a targeting construct as described herein further comprises a nucleic acid sequence or gene of particular interest, a selectable marker, control and/or regulatory sequences, and other nucleic acid sequences that allow for recombination mediated through exogenous addition of proteins that aid in or facilitate recombination involving such sequences. In some embodiments, a targeting construct as described herein further comprises a gene of interest in whole or in part, wherein the gene of interest is a heterologous gene that encodes a polypeptide, in whole or in part, that may have a similar function as a protein encoded by an endogenous sequence. In some embodiments, a targeting construct as described herein further comprises a gene of interest in whole or in part, wherein the gene of interest is a heterologous gene that encodes a polypeptide, in whole or in part, that has one or more different functions compared to a protein encoded by an endogenous sequence. In some embodiments, a targeting construct as described herein further comprises a humanized gene of interest, in whole or in part, wherein the humanized gene of interest encodes a polypeptide, in whole or in part, that may have a similar function as a polypeptide encoded by an endogenous sequence. In some embodiments, a targeting construct as described herein further comprises a humanized gene of interest, in whole or in part, wherein the humanized gene of interest encodes a polypeptide (e.g., human ADAR1), in whole or in part, that has one or more different functions compared to a polypeptide encoded by an endogenous sequence (e.g., mouse ADAR1). In some embodiments, a targeting construct (or targeting vector) may comprise a nucleic acid sequence manipulated by the hand of man. For example, in some embodiments, a targeting construct (or targeting vector) may be constructed to contain an engineered or recombinant polynucleotide that contains two or more sequences that are not linked together in that order in nature yet manipulated by the hand of man to be directly linked to one another in the engineered or recombinant polynucleotide.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a provided compound, e.g., a provided oligonucleotide.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Transgene or transgene construct: as used herein, refers to a nucleic acid sequence (encoding e.g., a polypeptide of interest, in whole or in part) that has been introduced into a cell by the hand of man such as by the methods described herein. A transgene could be partly or entirely heterologous, i.e., foreign, to the genetically engineered animal or cell into which it is introduced. A transgene can include one or more transcriptional regulatory sequences and any other nucleic acid, such as introns or promoters, which may be necessary for expression of a selected nucleic acid sequence.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Vector: as used herein, refers to a nucleic acid molecule capable of transporting another nucleic acid to which it is associated. In some embodiment, vectors are capable of extra-chromosomal replication and/or expression of nucleic acids to which they are linked in a host cell such as a eukaryotic and/or prokaryotic cell. Vectors capable of directing the expression of operably linked genes are referred to herein as “expression vectors.”

Wild-type: as used herein, refers to an entity having a structure and/or activity as found in nature in a “normal” (as contrasted with mutant, diseased, altered, engineered, transgenic etc.) state or context. Those of ordinary skill in the art will appreciate that wild-type genes and polypeptides often exist in multiple different forms (e.g., alleles).

As those skilled in the art will appreciate, methods and compositions described herein relating to provided compounds and/or characterization of provided compounds (e.g., oligonucleotides) generally also apply to pharmaceutically acceptable salts of such compounds

### DETAILED DESCRIPTION OF CERTAIN EMBODIMENTS

Among other things, the present disclosure encompasses the recognition that certain animals (e.g., mouse) and cells thereof may not be readily utilized as models for assessing agents and compositions for nucleic acid editing, e.g., editing of adenosines in transcripts (e.g., those G to A mutations). For example, in some embodiments, agents and compositions that can provide activities in human systems (e.g., human cells) demonstrated no or greatly reduced activities in animals (e.g. mice) whose endogenous ADAR proteins can be significantly different from human ADAR proteins.

In some embodiments, the present disclosure provides engineered animals and cell thereof, wherein the animals are engineered to comprise or express an ADAR1 polypeptide or a characteristic portion thereof, and/or a polynucleotide encoding such an ADAR1 polypeptide or a characteristic portion thereof. In some embodiments, after such an ADAR1 polypeptide or a characteristic portion thereof is introduced into engineered animals or cells, engineered animals or cells can demonstrate increased editing levels of one or more targets when editing agents, e.g., oligonucleotides, are administered compared to animals or cells not so engineered. In some embodiments, editing levels of one or more targets are comparable to, correlate to or parallel with those observed in reference human cells (e.g., cells of the same type). Those skilled in the art appreciate that various agents, including various oligonucleotide compositions described herein, can provide editing in human cells, and may be utilized to assess if a particular ADAR1 polypeptide or a characteristic portion thereof is suitable for engineering animals or cells (e.g., based on editing levels observed in engineered animals or cells expressing such an ADAR1 polypeptide or a characteristic portion thereof), if animals or cells shall be engineered (e.g., comparing activities of various agents in such animals or cells to those observed in human systems), or if engineered animals or cells are suitable for assessing activities of agents for editing activities (e.g., by assessing in such animals or cells activities of various agents (including active and/or inactive ones) and comparing to activities observed in human systems).

As described herein, in some embodiments, an ADAR1 polypeptide or a characteristic portion thereof is or comprises one or more or all of the following domains of a primate (e.g., human) ADAR1: Z-DNA binding domains, dsRNA binding domains, and deaminase domain. In some embodiments, an ADAR1 polypeptide or a characteristic portion thereof is or comprises one or both of a primate (e.g., human) ADAR1 Z-DNA binding domains; alternatively or additionally, in some embodiments, an ADAR1 polypeptide or a characteristic portion thereof is or comprises one, two or all of a primate (e.g., human) ADAR1 dsRNA binding domains; alternatively or additionally, an ADAR1 polypeptide or a characteristic portion thereof is or comprises a primate (e.g., human) deaminase domain. In some embodiments, a primate (e.g., human) ADAR1 polypeptide or a characteristic portion thereof may be expressed together with a non-primate (e.g., a rodent such as a mice) ADAR1 polypeptide or a characteristic portion thereof, e.g., one or more human dsRNA binding domains may be engineered to be expressed together with a mouse ADAR1 deaminase domain to form a human-mouse hybrid ADAR1 polypeptide. In some embodiments, an ADAR1 polypeptide or a characteristic portion thereof is or comprises a non-primate (e.g., rodent (e.g., mouse)) ADAR1, wherein a non-primate ADAR1 is engineered to have one or more of its domains replaced with one or more corresponding primate (e.g., human) ADAR1 domains (e.g., Z-DNA binding domains, dsRNA binding domains, and/or deaminase domains). In some embodiments, an ADAR1 polypeptide or a characteristic portion thereof is or comprises a human ADAR1. In some embodiments, an ADAR1 polypeptide or a characteristic portion thereof is or comprises human ADAR1 p110. In some embodiments, an ADAR1 polypeptide or a characteristic portion thereof is human ADAR1 p110. In some embodiments, an ADAR1 polypeptide or a characteristic portion thereof is or comprises human ADAR1 p150. In some embodiments, an ADAR1 polypeptide or a characteristic portion thereof is human ADAR1 p150.

In some embodiments, an ADAR1 polypeptide or a characteristic portion thereof is or comprises a sequence that shares about 80-100%, e.g., about or at least about 80%, 81%, 82%, 83%, 84%, 85%, 86%, 87%, 88%, 89%, 90%, 91%, 92%, 93%, 94%, 95%, 96%, 97%, 98%, 99%, 100% identity with a primate, e.g., a human ADAR1 or a characteristic portion thereof. In some embodiments, an ADAR1 polypeptide or a characteristic portion thereof is or comprises a sequence that shares about 80-100%, e.g., about or at least about 80%, 81%, 82%, 83%, 84%, 85%, 86%, 87%, 88%, 89%, 90%, 91%, 92%, 93%, 94%, 95%, 96%, 97%, 98%, 99%, 100% identity with one or more domains of human ADAR1. In some embodiments, regardless of amino acid sequence homology, an ADAR1 polypeptide or a characteristic portion thereof comprises a sequence or structure that shares one or more functions with a characteristic portion and/or one or more domains of human ADAR1. In some embodiments, one or more domains are or comprise one or more Z-DNA binding domain. In some embodiments, one or more domains are or comprise one or more or all dsRNA binding domain. In some embodiments, one or more domains are or comprise a deaminase domain.

In some embodiments, an animal is a rodent. In some embodiments, an animal is a rat. In some embodiments, an animal is a mouse.

Among other things, the present disclosure provides the insight that expression of human Adenosine Deaminase Acting on RNA 1 (ADAR1) in non-human animals can be exploited to generate model organisms useful for assessment and characterization of various editing agents, e.g., oligonucleotides, for various applications including therapeutic uses. Among other things, such animals can generate enhanced RNA editing which is more similar to that in human systems in reaction to editing agents such as oligonucleotides compared to animals not so engineered. Such editing agents, e.g., oligonucleotides, can be utilized to alter a functional (e.g., coding sequence, regulatory element etc.) sequence of a target RNA. In some embodiments, engineered non-human animals as described herein can provide an effective and efficient platform for assessing editing agents and/or developing human therapeutic agents. In some embodiments, the present disclosure provides genetically modified non-human animals that are able to express human ADAR1 for RNA editing.

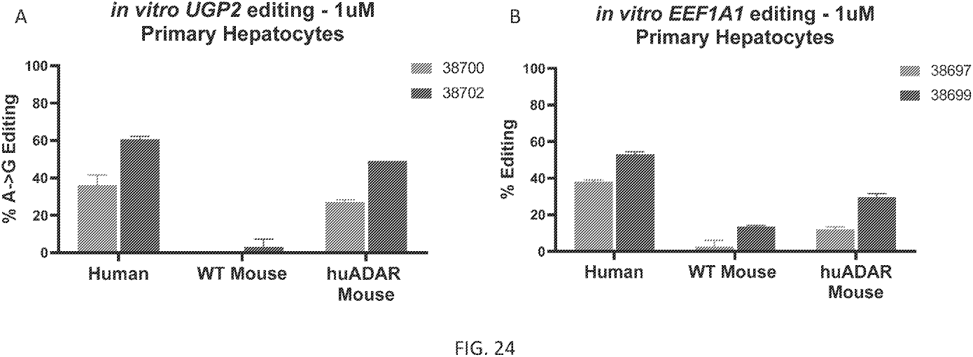

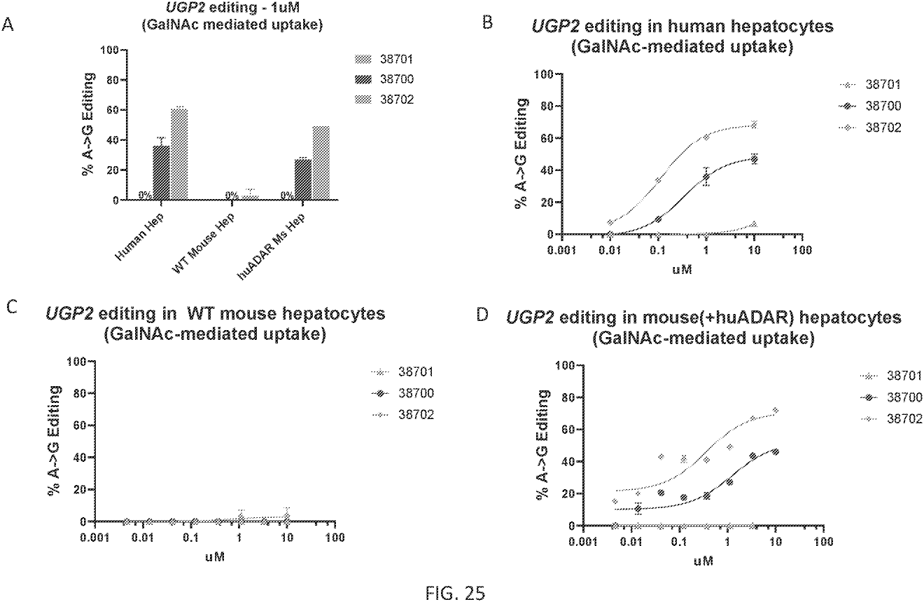

Among other things, the present disclosure recognizes that the characterization of various agents including oligonucleotides for site-directed RNA editing in non-human animals faces various challenges, as agents, e.g., oligonucleotides which elicit robust RNA editing events in human cells may fail to generate a comparable effect in non-human models (e.g., rodents, e.g., rats or mice). For example, mice treated with oligonucleotides for site-directed editing of UGP2 utilizing endogenous mouse ADAR1 often fail to create an editing response comparable to those observed in human cell lines (see FIGS. 24 and 25). The present disclosure further recognizes that the production of human ADAR1 (huADAR1) in a non-human animal can provide important in vivo data for the characterization of specific editing events, agents, and/or diseases related to abnormal RNA editing.

Various technologies may be utilized in accordance with the present disclosure to incorporated an ADAR1 polypeptide or a characteristic portion thereof into cells and non-human animals, e.g., through introduction of a polynucleotide whose sequence encoding an ADAR1 polypeptide or a characteristic portion thereof. In some embodiments, a polynucleotide is introduced into genomes of cells and non-human animals. In some embodiments, a polynucleotide is introduced into germline genomes of cells and non-human animals. As appreciated by those skilled in the art, various technologies for producing transgenic rodents (e.g., mice or rats) may be utilized in accordance with the present disclosure. In some embodiments, transgenic rodents (e.g., mice or rats) are produced via pronuclear injection of a polynucleotide into a single cell (e.g., a zygote) of a rodent (e.g., mouse or rat) embryo, where it will integrate into a rodent (e.g., mouse) genome (e.g., potentially randomly and/or in a site directed method). In some embodiments, this method creates a transgenic rodent (e.g., mice or rat) and is used to insert new genetic information into the genome or to over-express endogenous genes. In certain embodiments, this method also allows the replacement, deletion, and/or modification of endogenous rodent genes. In some embodiments, an alternative method of generating a transgenic rodent involves modifying embryonic stem cells with a DNA construct containing DNA sequences (e.g., for random genomic insertion and/or in a site directed manner). Embryonic stem cells that recombine with the genomic DNA are selected for, and are then injected into a mice blastocysts. In some embodiments, an alternative method of generating a transgenic rodent involves site-specific recombination using Cre-Lox recombination technology that involves the targeting and splicing out of a specific gene with the help of a recombinase. Cre is expressed in a specific cell type, creating a cell-type specific deletion of the targeted gene. This method requires mating Cre mice and floxed (sandwich the targeted gene with loxP sequences) mice to produce conditional knockout mice with the targeted gene deleted in certain cell type

Among other things, the present disclosure demonstrates that when various editing agents, e.g., oligonucleotides, are administered, engineered cells and/or non-human animals (e.g., mouse) comprising and/or expressing ADAR1 polypeptides or characteristic portions thereof (e.g., human ADAR1 (e.g., p110)) can unexpectedly provide editing much more similar or correlated to those observed in human cells (e.g., in quality and/or quantity, or patterns/trends of multiple agents/conditions, etc.) compared to cells and/or non-human animals not so engineered. In some embodiments, such cells and non-human animals are surprisingly useful for assessing, characterizing, identifying, and/or developing various editing agents, e.g., various oligonucleotides targeting adenosine.

In some embodiments, the present disclosure provides genetically modified non-human animals (e.g., rodents, e.g., mice) that express huADAR1 coding transcripts, including the highly relevant transcript variant 4 (encoding ADAR1 p110 protein), and transcript variant 1 (encoding ADAR1 p150 protein) coding sequences.

In some embodiments, methods for generating non-human animals expressing ADAR1 (e.g., of human or non-human primate) or characteristic portions thereof are characterized herein. In some embodiments, methods for utilizing said transgenic animals are described herein.

In some embodiments, cells and non-human animals expressing a primate, e.g., human, ADAR1 polypeptide or a characteristic portion thereof (e.g., rodents, e.g., rats or mice) are useful for characterizing, identifying and/or developing various agents, e.g., oligonucleotides, that can direct a correction of a G to A mutation in a target sequence or a product thereof, e.g., via ADAR-mediated deamination. In some embodiments, provided agents, e.g., oligonucleotides can direct a correction of a G to A mutation in a target sequence or a product thereof via ADAR-mediated deamination by recruiting a human ADAR1 (huADAR1), and facilitating the ADAR-mediated deamination. Regardless, however, the present disclosure is not limited to any particular mechanism. In some embodiments, the present disclosure provides non-human animals (e.g., rodents, e.g., rats or mice), oligonucleotides, compositions, methods, etc., useful for characterizing various RNA metabolism related pathways, such as but not limited to: double-stranded RNA interference, single-stranded RNA interference, RNase H-mediated knock-down, steric hindrance of translation, innate immunity, and/or a combination of two or more such pathways.

In some embodiments, methods for characterizing oligonucleotides suitable for directing site-specific ADAR1 editing are described. In some embodiments, oligonucleotides may contain portions that are not designed for complementarity (e.g., loops, protein binding sequences, etc., for recruiting of proteins, e.g., ADAR). In some embodiments, characterized oligonucleotides may hybridize to their target nucleic acids (e.g., pre-mRNA, mature mRNA, etc.). In some embodiments, oligonucleotides can hybridize to a target RNA sequence nucleic acid in any stage of RNA processing, including but not limited to a pre-mRNA or a mature mRNA. In some embodiments, oligonucleotide can hybridize to any element of a nucleic acid or its complement, including but not limited to: a promoter region, an enhancer region, a transcriptional stop region, a translational start signal, a translation stop signal, a coding region, a non-coding region, an exon, an intron, an intron/exon or exon/intron junction, the 5′ UTR, or the 3′ UTR.

### Adenosine Deaminases Acting on RNA

The modification of adenosine-to-inosine (A-to-I) is reported to be one of the most common mRNA-associated base modifications in humans, with an estimated ~1.6 million editing sites spread across the human transcriptome. A-to-I editing of RNA leads to deamination of adenosine to inosine. Inosine can be generally interpreted as guanosine by various cellular machinery, thus altering the coding, folding, splicing, and/or transport of transcripts. It has been reported that endogenous A-to-I editing is tightly regulated and this modification process is carried out by a highly conserved enzymatic family known as adenosine deaminases actin on RNA (ADARs), enzymes that are reported to be active throughout the metazoan kingdom and which are reported to be essential for the viability of certain mammals. It has been reported that altered editing may have severe consequences for human health and can cause influence interferonopathies, neurological disorders, cardiovascular disease, and cancer progression. There are reports that the ADAR enzyme family is considered highly conserved, and many ADARs follow a similar structural layout, with a variable number of amino (N) terminal double-stranded RNA binding domains (dsRBD) and a carboxyl (C) terminal deaminase domain. In addition to the canonical domains, human ADAR1 also contains either one or two Z-DNA binding domains. In humans, there are three known loci encoding functional ADAR enzymes, ADAR1, ADAR2, and the non-catalytically active ADAR3.

In humans and many eukaryotic organisms, Adenosine Deaminase Acting on RNA 1 (ADAR1) is reported to be responsible for the bulk of RNA editing events, and ADAR1-mediated RNA editing is reported to play an important role in antiviral immunity and may be essential for distinguishing between endogenous and viral RNA, thereby preventing autoimmune disorders. In humans, the ADAR1 protein has been reported to have two major isoforms (often referred to as long p150 and short p110) resulting from alternative promoters and start codons. ADAR1 p150 is reported to be induced by interferon, whereas ADAR1 p110 is reported to be relatively ubiquitously expressed.

In some embodiments, ADARs can bind to dsRNA targets and act in a processive manner, sequentially deaminating certain adenosines. In some embodiments, ADARs can bind to a dsRNA target and act in a specific and precise manner to edit only certain adenosines. Exogenously directing the function of endogenous ADAR1-mediated A-to-I RNA editing through the use of therapeutic agents may be used to correct genomic mutations at the RNA level, and may also be used to modulate tumor antigenicity. In some embodiments, ADAR enzymes can be guided to certain RNA sequences through the use of exogenously supplied oligonucleotides (e.g., RNA and/or modified versions thereof). In some embodiments titration of a supplied oligonucleotide may lead to a responsive change in site-directed RNA editing levels.

### Oligonucleotides and Oligonucleotide Compositions

In some embodiments, agents that capable of provide editing (e.g., A to I editing) are oligonucleotide agents. In some embodiments, the following oligonucleotides and compositions are described in the present disclosure. In some embodiments, a composition is a chirally controlled oligonucleotide composition. In a chirally controlled oligonucleotide composition of an oligonucleotide, the composition is enriched, compared to a stereorandom preparation of the oligonucleotide, for the oligonucleotide. As demonstrated herein, oligonucleotides and compositions thereof can provide adenosine editing when administered to cells and/or animals comprising or expressing a suitable ADAR1 polypeptide or a characteristic portion thereof. In some embodiments, oligonucleotides and compositions thereof may be utilized to assess/characterize ADAR1 polypeptides or characteristic portions thereof, or cells or non-human animals engineered to express an ADAR1 polypeptide or a characteristic portion thereof. In some embodiments, assessment and/or characterization comprises comparing editing levels in cells and/or animals engineered to comprise or express an ADAR1 polypeptide or a characteristic portion thereof, in cells and/or animals not so engineered, and/or in corresponding human systems (e.g., comparable cells and/or tissues, (e.g., the same type of cells and/or tissues), etc.). In some embodiments, particularly useful are ADAR1 polypeptides or characteristic portions thereof, and cells and non-human animals engineered to comprise and/or express such ADAR1 polypeptides or characteristic portions thereof, that can provide editing levels, profiles, patterns, etc. (from one or more agents) similar or comparable to corresponding human systems (e.g., qualitatively and/or quantitatively), particularly more similar or comparable when compared to corresponding ADAR1 polypeptides or characteristic portions thereof that are expressed prior to engineering, or cells and non-human animals prior to engineering.

TABLE 1

Certain useful oligonucleotides and compositions are described below:

| | | | | |
|---|---|---|---|---|
| ID | Description | Base Sequence | Stereochemistry/Linkage | Target |
| WV-38697 | Mod001L001fC^{∗}SfC^{∗}SfA^{∗}SfA^{∗}SfC^{∗}SfC^{∗}SfA ^{∗}SfG^{∗}SfA^{∗}SfA^{∗}SfA SfU^{∗}SfU^{∗}SfG^{∗}SfG^{∗}Sm C^{∗}SmA^{∗}SmC^{∗}SmA^{∗}SmA^{∗}SmA^{∗}SmU^{∗}SmG^{∗} SfC^{∗}SC^{∗}RA^{∗}RmC^{∗}SmU^{∗}SmG^{∗}SmU | CCAACCAGAA AUUGGCACAA AUGCCACUGU | OSSSSSSSSSSSS SSSSSSSSSSSSR RSSS | EEF1A1 |
| WV-38698 | Mod001L001fC^{∗}fC^{∗}fA^{∗}fA^{∗}fC^{∗}fC^{∗}fA^{∗}fG^{∗}fA ^{∗}fA^{∗}fAfU^{∗}fU^{∗}fG^{∗}fG^{∗}mC^{∗}mA^{∗}mC^{∗}mA^{∗}m A^{∗}mA^{∗}mU^{∗}mG^{∗}fC^{∗}C^{∗}A^{∗}mC^{∗}mU^{∗}mG^{∗}mU | CCAACCAGAA AUUGGCACAA AUGCCACUGU | OXXXXXXXXX XXXXXXXXXX XXXXXXXXXX | EEF1A1 |
| WV-38699 | Mod001L001fCn001RFC^{∗}SfA^{∗}SfA^{∗}SfC^{∗}SfC^{∗} SfA^{∗}SfG^{∗}SfA^{∗}SfA^{∗}SfA^{∗}SfU^{∗}SfU^{∗}SfGn001 RfG^{∗}SmCn001RmA*SmC^{∗}SmA^{∗}SmA^{∗}SmA ^{∗}SmU^{∗}SmG^{∗}SfC^{∗}SC^{∗}SAn001RmC^{∗}SmU^{∗}S | CCAACCAGAA AUUGGCACAA AUGCCACUGU | OnRSSSSSSSSSS SSnRSnRSSSSSS SSSnRSSnR | EEF1A1 |
| | mGn001RmU | | | |
| WV-38700 | Mod001L001fA1^{∗}SfU^{∗}SfC^{∗}SfC^{∗}SfA^{∗}SfC^{∗}Sf U^{∗}SfG^{∗}SfU^{∗}SfG^{∗}SfG^{∗}SfC^{∗}SfA^{∗}SfC^{∗}SfC^{∗}S mC^{∗}SmA^{∗}SmG^{∗}SmA^{∗}SmU^{∗}SmU^{∗}SmA^{∗}Sm U^{∗}SfC^{∗}SC^{∗}RA^{∗}RmU^{∗}SmG^{∗}SmU^{∗}SmU | AUCCACUGUG GCACCCAGAU UAUCCAUGUU | OSSSSSSSSSSSS SSSSSSSSSSSSR RSSS | UGP2 |
| WV-38701 | Mod001L00lfA^{∗}fU^{∗}fC^{∗}fC^{∗}fA^{∗}fC^{∗}fU^{∗}fG^{∗}fU ^{∗}fG^{∗}fG^{∗}fC^{∗}fA^{∗}fC^{∗}fC^{∗}mC^{∗}mA^{∗}mG^{∗}mA^{∗}m U^{∗}mU^{∗}mA^{∗}mU^{∗}fC^{∗}C^{∗}A^{∗}mU^{∗}mG^{∗}mU^{∗}mU | AUCCACUGUG GCACCCAGAU UAUCCAUGUU | OXXXXXXXXX XXXXXXXXXX XXXXXXXXXX | UGP2 |
| WV-38702 | Mod001L001fAn001RfU^{∗}SfC^{∗}SfC^{∗}SfA^{∗}SfC ^{∗}SfU^{∗}SfG^{∗}SfU^{∗}SfG^{∗}SfG^{∗}SfC^{∗}SfA^{∗}SfCn001 RfC^{∗}SmCn001RmA^{∗}SmG^{∗}SmA^{∗}SmU^{∗}SmU ^{∗}SmA^{∗}SmU^{∗}SfC^{∗}SC^{∗}SAn001RmU^{∗}SmG^{∗}S mUn001RmU | AUCCACUGUG GCACCCAGAU UAUCCAUGUU | OnRSSSSSSSSSS SSnRSnRSSSSSS SSSnRSSnR | UGP2 |
| WV-40590 | fAn001RfU^{∗}SfC^{∗}SfC^{∗}SfA^{∗}SfC^{∗}SfU^{∗}SfG^{∗}Sf U^{∗}SfG^{∗}SfG^{∗}SfC^{∗}SfA^{∗}SfCn001RfC^{∗}SmCn00 1RmA^{∗}SmG^{∗}SmA^{∗}SmU^{∗}SmU^{∗}SmA^{∗}SmU^{∗} SfC^{∗}SC^{∗}SAn001RmU^{∗}SmG^{∗}SmUn001RmU | AUCCACUGUG GCACCCAGAU UAUCCAUGUU | nRSSSSSSSSSSS SnRSnRSSSSSSS SSnRSSnR | UGP2 |
| WV-40592 | fUn001RfU^{∗}SfA^{∗}SfA^{∗}SfU^{∗}SfC^{∗}SfC^{∗}SfA^{∗}Sf U^{∗}SfC^{∗}SfU^{∗}SfC^{∗}SfU^{∗}SfUn001RfC^{∗}SmAn00 1RmG^{∗}SmA^{∗}SmU^{∗}SmA^{∗}SmU^{∗}SmG^{∗}SmU^{∗} SfC^{∗}SC^{∗}SAn001RmC^{∗}SmA^{∗}SmGn001RmA | UUAAUCCAUC UCUUCAGAUA UGUCCACAGA | nRSSSSSSSSSSS SnRSnRSSSSSSS SSnRSSnR | SRSF1 |

### Notes:

Description, Base Sequence and Stereochemistry/Linkage, due to their length, may be divided into multiple lines in Table 1 (e.g., Table 1A, Table 1B and Table 1C). Unless otherwise specified, all oligonucleotides in Table 1 are single-stranded. As appreciated by those skilled in the art, nucleoside units are unmodified and contain unmodified nucleobases and 2′-deoxy sugars unless otherwise indicated (e.g., with r, m, m5, eo, etc.); linkages, unless otherwise indicated, are natural phosphate linkages; and acidic/basic groups may independently exist in their salt forms. If a sugar is not specified, the sugar is a natural DNA sugar; and if an internucleotidic linkage is not specified, the internucleotidic linkage is a natural phosphate linkage. Moieties and modifications:
m: 2′-OMe;
f: 2′-F;
O, PO: phosphodiester (phosphate). It can a linkage or be an end group (or a component thereof), e.g., a linkage between a linker and an oligonucleotide chain, an internucleotidic linkage (a natural phosphate linkage), etc. Phosphodiesters are typically indicated with “O” in the Stereochemistry/Linkage column and are typically not marked in the Description column (if it is an end group, e.g., a 5′-end group, it is indicated in the Description and typically not in Stereochemistry/Linkage); if no linkage is indicated in the Description column, it is typically a phosphodiester unless otherwise indicated. Note that a phosphate linkage between a linker (e.g., L001) and an oligonucleotide chain may not be marked in the Description column, but may be indicated with “O” in the Stereochemistry/Linkage column;
^{∗}, PS: Phosphorothioate. It can be an end group (if it is an end group, e.g., a 5′-end group, it is indicated in the Description and typically not in Stereochemistry/Linkage), or a linkage, e.g., a linkage between linker (e.g., L001) and an oligonucleotide chain, an internucleotidic linkage (a phosphorothioate internucleotidic linkage), etc.;
R, Rp: Phosphorothioate in the Rp configuration. Note that * R in Description indicates a single phosphorothioate linkage in the Rp configuration;
S, Sp: Phosphorothioate in the Sp configuration. Note that * S in Description indicates a single phosphorothioate linkage in the Sp configuration;
X: stereorandom phosphorothioate;
n001:

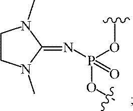

nX: stereorandom n001;
nR or n001R: n001 in Rp configuration;
nS or n001S: n001 in Sp configuration;
Mod001:

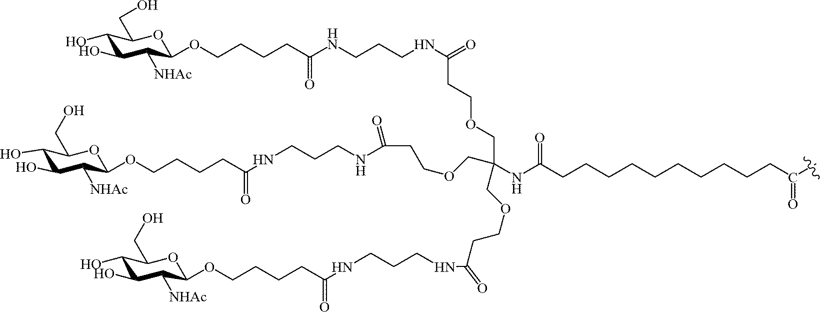

L001 : -NH-(CH₂)₆- linker (C6 linker, C6 amine linker or C6 amino linker), connected to Mod (e.g., Mod001) through —NH—, and, in the case of, for example, WV-38697, the 5′-end of the oligonucleotide chain through a phosphate linkage (O or PO). For example, in WV-38697, L001 is connected to Mod001 through —NH— (forming an amide group —C(O)—NH—), and is connected to the oligonucleotide chain through a phosphate linkage (O).

### Engineered Animals, Cells and Tissues

Non-human animals are provided that are engineered to comprise and/or express an exogenous ADAR1 polypeptide or a characteristic portion thereof (e.g., whose somatic and/or germline tissues comprise a polynucleotide whose sequence encoding an ADAR1 polypeptide or a characteristic portion thereof). In some embodiments, a polynucleotide encoding an exogenous ADAR1 polypeptide or a characteristic portion thereof in genomes of provided cells, tissues, or non-human animals. In some embodiments, such a polynucleotide is germline genome of non-human animals. In various embodiments described herein, a genetically modified non-human animal is a rodent, such as a rat or a mouse, and non-human elements described herein (enhancers, constant regions, etc.) are rodent, such as rat or mouse elements. Suitable examples of non-human animals described herein include, but are not limited to, rodents, for example, rats or mice, in particular, mice.

In some embodiments, the present disclosure provides improved in vivo systems for identifying and developing new and/or characterizing known agents such as oligonucleotides for in vivo and/or in vitro site-directed RNA editing mediated by ADAR1. Developed oligonucleotides can be used, for example, in the treatment of a variety of diseases that affect humans. Further, the present disclosure encompasses the recognition that non-human animals (e.g., rodents, e.g. rats or mice) having engineered human ADAR1 loci, such as an engineered human ADAR1, are useful. In some embodiments, non-human animals described herein provide improved in vivo systems for development of oligonucleotides or oligonucleotide-based therapeutics for administration to humans. In some embodiments, non-human animals described herein provide improved in vivo systems for development of oligonucleotides or oligonucleotide-based therapeutics characterized by improved and/or different performance (e.g., target RNA editing levels) as compared to oligonucleotides or oligonucleotide-based therapeutics characterized from existing in vivo rodent systems that do not comprise human ADAR1 coding region sequences.

The present disclosure provides, among other things, a non-human animal (e.g., rodent, e.g., rat or mouse), non-human (e.g., rodent, e.g., rat or mouse) cell or non-human (e.g., rodent, e.g., rat or mouse) tissue having an endogenous locus that has been engineered to include a human ADAR1 coding region or characteristic portion thereof. In some embodiments, sequences of a human ADAR1 coding region are operably linked to a non-human regulatory region.

The present disclosure provides, among other things, a non-human animal (e.g., rodent, e.g., rat or mouse), non-human (e.g., rodent, e.g., rat or mouse) cell or non-human (e.g., rodent, e.g., rat or mouse) tissue having an endogenous locus that has been engineered to include a non-human primate (NHP) ADAR coding region or characteristic portion thereof. In some embodiments, sequences of a NHP ADAR coding region are operably linked to a non-human regulatory region.

In some embodiments, a non-human ADAR gene is or comprises a mammalian ADAR gene selected from the group consisting of a primate, goat, sheep, pig, dog, cow, or rodent (e.g., rat or mouse) ADAR gene.

In some embodiments, a non-human ADAR is or comprises a primate ADAR1 polypeptide or a characteristic portion thereof.

In some embodiments, a non-human animal (e.g., rodent, e.g., rat or mouse), non-human (e.g., rodent, e.g., rat or mouse) cell or non-human (e.g., rodent, e.g., rat or mouse) tissue described herein includes an endogenous ADAR1 gene in its genome (e.g., its germline genome), which encodes an ADAR1 polypeptide, functional ortholog, functional homolog, or functional fragment thereof. In some embodiments, a non-human animal (e.g., rodent, e.g., rat or mouse), non-human (e.g., rodent, e.g., rat or mouse) cell or non-human (e.g., rodent, e.g., rat or mouse) tissue described herein includes an endogenous ADAR1 gene in its genome (e.g., its germline genome) that is no longer functioning in a WT manner, e.g., it is deleted, replaced, and/or mutated in such a way to generate a hypomorphic and/or null allele. In some embodiments, a non-human animal (e.g., rodent, e.g., rat or mouse), non-human (e.g., rodent, e.g., rat or mouse) cell or non-human (e.g., rodent, e.g., rat or mouse) tissue described herein includes an additional ADAR1 gene in its genome (e.g., its germline genome), which encodes an additional rodent ADAR1 polypeptide, functional ortholog, functional homolog, or functional fragment thereof. In some embodiments, an engineered animal or a cell thereof does not contain or express its wild-type ADAR1. In some embodiments, in an engineered animal or a cell thereof its original ADAR1 is replaced with an ADAR1 polypeptide or a characteristic portion thereof described in, e.g., one that comprises a primate, e.g., human ADAR1 polypeptide or a characteristic portion thereof.

In some embodiments, an engineered non-human animal or a cell thereof comprises or expresses an ADAR1 polypeptide which comprises one or more domains of a primate, e.g., human ADAR1 or a characteristic element sequence thereof.

In some embodiments, an engineered cell, tissue or non-human animal comprises and/or expresses a polynucleotide whose sequence encodes an ADAR1 polypeptide or a characteristic portion thereof. In some embodiments, such a polynucleotide comprises one or more introns. In some embodiments, a polypeptide encoded by such a polynucleotide comprises one or more domains or characteristic portions of a primate, e.g., human ADAR1. In some embodiments, a polypeptide encoded by such a polynucleotide comprises one or more portions that can perform one or more functions of one or more domains or characteristic portions of a primate, e.g., human ADAR1, which one or more functions cannot be performed, or cannot be performed at comparable levels, by the one or more corresponding mouse portions. In some embodiments, a polypeptide encoded by such a polynucleotide can perform one or more functions of one or more domains or characteristic portions of a primate, e.g., human ADAR1, which one or more functions cannot be performed, or cannot be performed at comparable levels, by a corresponding mouse ADAR1.

In some embodiments, a polypeptide encoded by such a polynucleotide comprises one or more portions that independently have levels of homology with one or more domains or characteristic portions of a primate, e.g., human, ADAR1 (e.g., human ADAR1 p110). In some embodiments, such an encoded polypeptide comprises one or more portions that independently have higher, compared to portions in an ADAR1 in a cell, tissue, animal, etc. not so engineered, levels of homology with one or more domains or characteristic portions of a primate, e.g., human, ADAR1 (e.g., human ADAR1 p110). In some embodiments, a homology is about or at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100%. In some embodiments, a homology is about or at least about 80%, 81%, 82%, 83%, 84%, 85%, 86%, 87%, 88%, 89%, 90%, 91%, 92%, 93%, 94%, 95%, 96%, 97%, 98%, 99% or 100%. In some embodiments, a homology is about or at least about 85%, 86%, 87%, 88%, 89%, 90%, 91%, 92%, 93%, 94%, 95%, 96%, 97%, 98%, 99% or 100%. In some embodiments, a homology is about or at least about 90%, 91%, 92%, 93%, 94%, 95%, 96%, 97%, 98%, 99% or 100%.

In some embodiments, a polypeptide encoded by such a polynucleotide has a level of homology with a primate, e.g., human, ADAR1 (e.g., human ADAR1 p110). In some embodiments, such an encoded polypeptide has a higher, compared to an ADAR1 in a cell, tissue, animal, etc. not so engineered, level of homology with a primate, e.g., human, ADAR1 (e.g., human ADAR1 p110). In some embodiments, a homology is about or at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100%. In some embodiments, a homology is about or at least about 80%, 81%, 82%, 83%, 84%, 85%, 86%, 87%, 88%, 89%, 90%, 91%, 92%, 93%, 94%, 95%, 96%, 97%, 98%, 99% or 100%. In some embodiments, a homology is about or at least about 85%, 86%, 87%, 88%, 89%, 90%, 91%, 92%, 93%, 94%, 95%, 96%, 97%, 98%, 99% or 100%. In some embodiments, a homology is about or at least about 90%, 91%, 92%, 93%, 94%, 95%, 96%, 97%, 98%, 99% or 100%.

In some embodiments, a non-human animal (e.g., rodent, e.g., rat or mouse), non-human (e.g., rodent, e.g., rat or mouse) cell or non-human (e.g., rodent, e.g., rat or mouse) tissue described herein includes an exogenous ADAR1 gene in its genome (e.g., its germline genome), which encodes a human ADAR1 polypeptide, functional ortholog, functional homolog, or functional fragment thereof. In some embodiments, a non-human animal (e.g., rodent, e.g., rat or mouse), non-human (e.g., rodent, e.g., rat or mouse) cell or non-human (e.g., rodent, e.g., rat or mouse) tissue described herein includes an exogenous ADAR1 gene in its genome (e.g., its germline genome), which encodes a non-human primate (NHP) ADAR1 polypeptide, functional ortholog, functional homolog, or functional fragment thereof. In some embodiments, a non-human animal (e.g., rodent, e.g., rat or mouse), non-human (e.g., rodent, e.g., rat or mouse) cell or non-human (e.g., rodent, e.g., rat or mouse) tissue described herein includes an exogenous ADAR1 gene in its genome (e.g., its germline genome), which encodes a chimeric ADAR1 polypeptide (e.g., encompassing features from more than one species, i.e. an adenosine deaminase domain derived from a human ADAR1 gene, and one or more double stranded RNA binding domains derived form a NHP ADAR1 gene), functional ortholog, functional homolog, or functional fragment thereof.

In some embodiments, an exogenous ADAR1 gene encoding a polypeptide, functional ortholog, functional homolog, or functional fragment thereof is expressed from an endogenous ADAR1 gene locus. In some embodiments, an exogenous ADAR1 gene in a genetically modified non-human animal as described herein does not originate from that specific non-human animal (e.g., a mouse that includes a human ADAR1 gene or a NHP ADAR1 gene). In some embodiments, a non-human animal described herein includes an ectopic exogenous ADAR1 gene. An “ectopic” ADAR1 locus, as used herein, refers to an ADAR1 locus that is in a different context than the endogenous ADAR1 gene appears in a wild-type non-human animal. For example, an exogenous ADAR1 gene could be located on a different chromosome, located at a different locus, or positioned adjacent to different sequences. An exemplary ectopic exogenous ADAR1 gene is a human ADAR1 p110 or p150 encoding locus located within a safe harbor loci, (e.g., the ROSA26 locus, the H11 locus, the TIGRE locus, and/or the MYH9 locus). In some embodiments, a non-human animal described herein includes an inserted or integrated ADAR1 gene.

In some embodiments, a non-human animal, non-human cell or non-human tissue described herein includes an insertion of one or more nucleotide sequences encoding one or more human ADAR1 polypeptides, functional orthologs, functional homologs, or functional fragments thereof in its genome (e.g., its germline genome).

In some embodiments, a non-human animal, non-human cell or non-human tissue described herein includes an insertion of one or more nucleotide sequences encoding one or more NHP ADAR1 polypeptides, functional orthologs, functional homologs, or functional fragments thereof in its genome (e.g., its germline genome).

In some embodiments, one or more nucleotide sequences encoding one or more ADAR1 polypeptides, functional orthologs, functional homologs, or functional fragments thereof are inserted and/or are located on the same chromosome as the endogenous mouse ADAR1 locus. In some embodiments, one or more nucleotide sequences encoding one or more human ADAR1 polypeptides, functional orthologs, functional homologs, or functional fragments thereof are inserted and/or are located on the same chromosome as the endogenous mouse ADAR1 locus. In some embodiments, one or more nucleotide sequences encoding one or more human ADAR1 polypeptides, functional orthologs, functional homologs, or functional fragments thereof are inserted and/or are located in a position so that the one or more nucleotide sequences encoding one or more human ADAR1 polypeptides, functional orthologs, functional homologs, or functional fragments thereof are contiguous with an endogenous mouse ADAR1 gene. In some embodiments, one or more nucleotide sequences encoding one or more human ADAR1 polypeptides, functional orthologs, functional homologs, or functional fragments thereof are inserted and/or are located in a position so that the one or more nucleotide sequences encoding one or more human ADAR1 polypeptides, functional orthologs, functional homologs, or functional fragments thereof are adjacent to an endogenous mouse ADAR1 gene. In some embodiments, one or more nucleotide sequences encoding one or more human ADAR1 polypeptides, functional orthologs, functional homologs, or functional fragments thereof are inserted and/or are located in a position so that the one or more nucleotide sequences encoding one or more human ADAR1 polypeptides, functional orthologs, functional homologs, or functional fragments thereof functionally replace an endogenous mouse ADAR1 gene.

In some embodiments, one or more nucleotide sequences encoding one or more ADAR1 polypeptides, functional orthologs, functional homologs, or functional fragments thereof are inserted and/or are located on the same chromosome as the endogenous mouse ADAR1 locus. In some embodiments, one or more nucleotide sequences encoding one or more NHP ADAR1 polypeptides, functional orthologs, functional homologs, or functional fragments thereof are inserted and/or are located on the same chromosome as the endogenous mouse ADAR1 locus. In some embodiments, one or more nucleotide sequences encoding one or more NHP ADAR1 polypeptides, functional orthologs, functional homologs, or functional fragments thereof are inserted and/or are located in a position so that the one or more nucleotide sequences encoding one or more NHP ADAR1 polypeptides, functional orthologs, functional homologs, or functional fragments thereof are contiguous with an endogenous mouse ADAR1 gene. In some embodiments, one or more nucleotide sequences encoding one or more NHP ADAR1 polypeptides, functional orthologs, functional homologs, or functional fragments thereof are inserted and/or are located in a position so that the one or more nucleotide sequences encoding one or more NHP ADAR1 polypeptides, functional orthologs, functional homologs, or functional fragments thereof are adjacent to an endogenous mouse ADAR1 gene. In some embodiments, one or more nucleotide sequences encoding one or more NHP ADAR1 polypeptides, functional orthologs, functional homologs, or functional fragments thereof are inserted and/or are located in a position so that the one or more nucleotide sequences encoding one or more NHP ADAR1 polypeptides, functional orthologs, functional homologs, or functional fragments thereof functionally replace an endogenous mouse ADAR1 gene.

In some embodiments, a non-human animal (e.g., rodent, e.g., rat or mouse), non-human (e.g., rodent, e.g., rat or mouse) cell or non-human (e.g., rodent, e.g., rat or mouse) tissue described herein includes an exogenous ADAR1 gene that restores or enhances ADAR activity in response to an exogenously supplied potentially therapeutic oligonucleotide.. In some embodiments, the exogenous ADAR1 gene restores ADAR editing activity in response to an oligonucleotide to the level comparable in a human cell and/or tissue that includes a functional, endogenous ADAR1 gene. In some embodiments, the exogenous ADAR1 gene restores ADAR editing activity in response to an oligonucleotide to the level slightly lower than in a human cell and/or tissue that includes a functional, endogenous ADAR1 gene. In some embodiments, the exogenous ADAR1 gene restores ADAR editing activity in response to an oligonucleotide to the level lower than in a human cell and/or tissue that includes a functional, endogenous ADAR1 gene.

In some embodiments, a non-human animal (e.g., rodent, e.g., rat or mouse), non-human (e.g., rodent, e.g., rat or mouse) cell or non-human (e.g., rodent, e.g., rat or mouse) tissue described herein includes an exogenous ADAR1 gene that enhances ADAR activity in response to an exogenously supplied potentially therapeutic oligonucleotide when compared to a WT animal which does not comprise an exogenous ADAR1 gene. In some embodiments, the exogenous ADAR1 gene facilitates ADAR editing activity in response to an oligonucleotide to a level significantly higher than that found in a non-human animal, tissue, and/or cell that does not express an exogenous ADAR1 gene. In some embodiments, the exogenous ADAR1 gene enhances ADAR activity to a level that is at least 2 times, at least 3 times, at least 4 times, at least 5 times, at least 6 times, at least 7 times, at least 8 times, at least 9 times, or at least 10 times the ADAR activity of a comparable WT animal that does not include an exogenous ADAR1 gene.

In some embodiments of a non-human animal (e.g., rodent, e.g., rat or mouse), non-human (e.g., rodent, e.g., rat or mouse) cell or non-human (e.g., rodent, e.g., rat or mouse) tissue as described herein, the non-human animal, non-human cell or non-human tissue is homozygous or heterozygous for an exogenous ADAR1 gene (e.g., as described herein, integrated at a known or a random locus).

In some embodiments of a non-human animal (e.g., rodent, e.g., rat or mouse), non-human (e.g., rodent, e.g., rat or mouse) cell or non-human (e.g., rodent, e.g., rat or mouse) tissue as described herein, the non-human animal, non-human cell or non-human tissue is homozygous or heterozygous for an exogenous ADAR1 gene integrated at the ROSA26 locus.

In some embodiments of a non-human animal (e.g., rodent, e.g., rat or mouse), non-human (e.g., rodent, e.g., rat or mouse) cell or non-human (e.g., rodent, e.g., rat or mouse) tissue as described herein, the non-human animal, non-human cell or non-human tissue is homozygous or heterozygous for an exogenous ADAR1 gene integrated at the H11 locus.

In some embodiments of a non-human animal (e.g., rodent, e.g., rat or mouse), non-human (e.g., rodent, e.g., rat or mouse) cell or non-human (e.g., rodent, e.g., rat or mouse) tissue as described herein, the non-human animal, non-human cell or non-human tissue is homozygous or heterozygous for an exogenous ADAR1 gene integrated at the TIGRE locus.

In some embodiments of a non-human animal (e.g., rodent, e.g., rat or mouse), non-human (e.g., rodent, e.g., rat or mouse) cell or non-human (e.g., rodent, e.g., rat or mouse) tissue as described herein, the non-human animal, non-human cell or non-human tissue is homozygous or heterozygous for an exogenous ADAR1 gene integrated at the MYH9 locus.

In some embodiments of a non-human animal (e.g., rodent, e.g., rat or mouse), non-human (e.g., rodent, e.g., rat or mouse) cell or non-human (e.g., rodent, e.g., rat or mouse) tissue as described herein, the non-human animal, non-human cell or non-human tissue is homozygous or heterozygous for an exogenous ADAR1 gene integrated at a locus amenable for manipulation using Cre-Lox P and/or Flp-FRT; see E.g., Kim et al., “Mouse Cre-LoxP system: general principles to determine tissue-specific roles of target genes” Laboratory Animal Research (2018) 34(4), 147-159.

In some embodiments of a non-human animal (e.g., rodent, e.g., rat or mouse), non-human (e.g., rodent, e.g., rat or mouse) cell or non-human (e.g., rodent, e.g., rat or mouse) tissue as described herein, the non-human animal, non-human cell or non-human tissue is homozygous or heterozygous for an exogenous ADAR1 gene integrated at a Cre-Lox P stop or inducible loxP-Cre site. In certain such embodiments, when crossed with a mouse that has Cre under a tissue specific promoter, said locus can generate tissue specific exogenous ADAR1 expression in transgenic animals.

In some embodiments of a non-human animal (e.g., rodent, e.g., rat or mouse), non-human (e.g., rodent, e.g., rat or mouse) cell or non-human (e.g., rodent, e.g., rat or mouse) tissue as described herein, the non-human animal, non-human cell or non-human tissue is homozygous or heterozygous for an exogenous ADAR1 gene integrated at a site operably linked to an inducible promoter (e.g., a tetracycline-responsive element, an estrogen receptor targeting motif, and/or under the control of tamoxifen).

In some embodiments of a non-human animal (e.g., rodent, e.g., rat or mouse), non-human (e.g., rodent, e.g., rat or mouse) cell or non-human (e.g., rodent, e.g., rat or mouse) tissue as described herein, the non-human animal, non-human cell or non-human tissue is homozygous or heterozygous for an exogenous ADAR1 gene integrated at a site operably linked to a universally expressed promoter (e.g., CMV, SV40, elongation factor 1 alpha, CBA/CAGG, ubiquitin C, and/or phosphoglycerate kinase 1).

In some embodiments of a non-human animal (e.g., rodent, e.g., rat or mouse), non-human (e.g., rodent, e.g., rat or mouse) cell or non-human (e.g., rodent, e.g., rat or mouse) tissue as described herein, the non-human animal, non-human cell or non-human tissue is homozygous or heterozygous for an exogenous ADAR1 gene integrated at a known site. In certain embodiments, integration of an exogenous ADAR1 gene was facilitated through the use of gene editing tools such as endonucleases. In certain embodiments, exogenous ADAR1 gene integration is facilitated using CRISPR/Cas9 targeting a known locus. In certain embodiments, exogenous ADAR1 gene integration is facilitated using TALENs targeting a known locus. In certain embodiments, exogenous ADAR1 gene integration is facilitated using Zinc Finger Nucleases targeting a known locus.

In some embodiments of a provided non-human animal (e.g., rodent, e.g., rat or mouse), non-human (e.g., rodent, e.g., rat or mouse) cell or non-human (e.g., rodent, e.g., rat or mouse) tissue, the endogenous ADAR1 locus is deleted in whole or in part. In some embodiments of a provided non-human animal, non-human cell or non-human tissue, the endogenous ADAR1 locus is functionally silenced or otherwise non-functional (e.g., by gene targeting). In some certain embodiments of a provided non-human animal, non-human cell or non-human tissue, the non-human animal, non-human cell or non-human tissue is homozygous for a functionally silenced or otherwise non-functional endogenous ADAR1 locus. In some embodiments, a non-human animal (e.g., rodent, e.g., rat or mouse), non-human (e.g., rodent, e.g., rat or mouse) cell or non-human (e.g., rodent, e.g., rat or mouse) tissue as described herein does not detectably express endogenous ADAR1 polypeptide. In some embodiments of a provided non-human animal (e.g., rodent, e.g., rat or mouse), non-human (e.g., rodent, e.g., rat or mouse) cell or non-human (e.g., rodent, e.g., rat or mouse) tissue, the endogenous ADAR1 locus is intact and is functional at least in part.

In some embodiments, a non-human animal, non-human cell or non-human tissue as described herein has a genome further comprising a nucleic acid sequence encoding an exogenous ADAR1 operably linked to a transcriptional and/or translational regulatory element.

In some embodiments, a transcriptional control element includes a splice acceptor element, a KOZAK sequence, a WPRE sequence, a poly(A) signal sequence, and/or any combination thereof.

In some embodiments, a non-human ADAR1 locus that is altered, displaced, disrupted, deleted, replaced or engineered with one or more exogenous ADAR1 gene sequences as described herein is a murine ADAR1 locus. In some embodiments, one or more human ADAR1 gene sequences as described herein are inserted into one copy (i.e., allele) of a non-human ADAR1 locus of the two copies of said non-human ADAR1 locus, giving rise to a non-human animal that is heterozygous with respect to the ADAR1 locus sequence (e.g., wherein one copy is from an exogenous ADAR1 gene, and the other copy is of an endogenous ADAR1 locus). In some embodiments, a non-human animal is provided that is homozygous for an exogenous ADAR1 gene that includes one or more ADAR1 sequences as described herein.

In some embodiments, one or more endogenous non-human ADAR1 sequences (or portions thereof) of an endogenous non-human ADAR1 locus are not deleted. In some embodiments, one or more endogenous ADAR1 sequences (or portions thereof) of an endogenous non-human ADAR1 locus are deleted. In some embodiments, one or more endogenous non-human ADAR1 sequences of an endogenous non-human ADAR1 locus is altered, displaced, disrupted, deleted or replaced so that said non-human ADAR1 locus is functionally silenced. In some embodiments, one or more endogenous non-human ADAR1 sequences of an endogenous non-human ADAR1 locus is altered, displaced, disrupted, deleted or replaced with a targeting vector so that said non-human ADAR1 locus is functionally inactivated (i.e., unable to produce a functional ADAR1 polypeptide that is expressed and/or detectable in the protein milieu of a non-human animal as described herein). Methods for inactivation of an endogenous gene are known in the art.

In some embodiments, an exogenous ADAR1 gene or transgene or its expression product can be detected using a variety of methods including, for example, PCR, Southern blot, restriction fragment length polymorphism (RFLP), a gain or loss of allele assay, Western blot, FACS analysis, etc. In some embodiments, a non-human animal, non-human cell or non-human tissue as described herein is heterozygous with respect to an exogenous ADAR1 gene as described herein. In some embodiments, a non-human animal, non-human cell or non-human tissue as described herein is hemizygous with respect to an exogenous ADAR1 gene as described herein. In some embodiments, a non-human animal, non-human cell or non-human tissue as described herein contains one or more copies of an exogenous ADAR1 gene or transgene as described herein.

The present disclosure recognizes that in some embodiments of a non-human animal (e.g., rodent, e.g., rat or mouse), non-human (e.g., rodent, e.g., rat or mouse) cell or non-human (e.g., rodent, e.g., rat or mouse) tissue as described herein, the animal, cell, or tissue will utilize the product of an exogenous ADAR1 gene integrated in its genome. As such, in various embodiments, exogenous ADAR1 loci integrated within a non-human animals, non-human cells or non-human tissues described herein may encode an exogenous ADAR1 gene that is hypomorphic.

The present disclosure provides, among other things, cells and tissues from non-human animals (e.g., rodents, e.g., rats, mice) described herein. In some embodiments, cells or tissues are hepatic cells or tissues. In some embodiments, cells or tissues are neuronal cells or tissues. In some embodiments, any cell or tissue from a non-human animal as described herein may be isolated. In some embodiments, provided is an isolated cell and/or an isolated tissue from a non-human animal as described herein. In some embodiments, an isolated cell may be immortalized.

Non-human animals (e.g., rodents, e.g., rats or mice) as described herein may be utilized for the characterization of one or more oligonucleotides of interest under conditions and for a time sufficient that the non-human animal develops and/or has the potential to develop a molecular response (e.g., RNA editing, transcriptional changes, translational changes, etc.) as a result of said oligonucleotide(s).

In some embodiments, a non-human animal (e.g., rodents, e.g., rats or mice) produces a population of cells that express human ADAR1 polypeptide that may bind to one or more RNA species of interest. In certain embodiments, a human ADAR1 polypeptide may bind to one or more RNA species of interest through interaction with a site-directing potentially therapeutic oligonucleotide. In some embodiments, human ADAR1 polypeptide binds to one or more RNA species of interest, and human ADAR1 polypeptide acts to edit said RNA molecule.

In some embodiments, a non-human animal (e.g., rodent, e.g., rat or mouse), non-human (e.g., rodent, e.g., rat or mouse) cell or non-human (e.g., rodent, e.g., rat or mouse) tissue as described herein comprises an exogenous ADAR1 gene integrated in its genome represented by SEQ ID NO: 3 or SEQ ID NO: 14.

In some embodiments, a non-human animal, e.g., a mouse, is engineered to comprise or express an engineered ADAR1, wherein one or more domains or portions of the animal’s (e.g., mouse) prior-to-engineering ADAR1 are independently replaced with one or more domains (e.g., one or more of Z-DNA binding domains, one or more or all dsRNA binding domains, and/or deaminase domain, etc.) or characteristic portions of a primate, e.g., human ADAR1 (e.g., human ADAR1 p110 or p150). In some embodiments, cells and/or animals expressing an engineered ADAR1 can provide higher levels of editing when editing agents, e.g., oligonucleotide compositions (e.g., those described herein) are administered.

### Nucleic Acid Constructs

Typically, a polynucleotide molecule containing an exogenous ADAR sequences (e.g., ADAR1, e.g., a human or NHP ADAR1 gene), or portion(s) thereof, is linked with (e.g., is inserted into) a vector, preferably a DNA vector, in order to replicate the polynucleotide molecule in a host cell.

ADAR sequences can be cloned directly from known sequences or sources (e.g., libraries) or synthesized from germline sequences designed in silico based on published sequences available from GenBank or other publically available databases. Alternatively, bacterial artificial chromosome (BAC) libraries can provide ADAR DNA sequences of interest (e.g., human ADAR1 sequences and/or characteristic portions thereof). BAC libraries can contain an insert size of 100-150 kb and are capable of harboring inserts as large as 300 kb (Shizuya, et al., 1992, Proc. Natl. Acad. Sci., USA 89:8794-8797; Swiatek, et al., 1993, Genes and Development 7:2071-2084; Kim, et al., 1996, Genomics 34 213-218). For example, a human BAC library harboring average insert sizes of 164-196 kb has been described (Osoegawa, K. et al., 2001, Genome Res. 11(3):483-96; Osoegawa, K. et al., 1998, Genomics 52:1-8, Article No. GE985423). Human and non-human animal genomic BAC libraries have been constructed and are commercially available (e.g., ThermoFisher). Genomic BAC libraries can also serve as a source of ADAR DNA sequences as well as transcriptional control regions.

Alternatively, ADAR1 DNA sequences may be isolated, cloned and/or transferred from yeast artificial chromosomes (YACs). For example, the nucleotide sequence of the human ADAR1 gene has been determined. An entire ADAR1 locus (human or non-human) can be cloned and contained within several YACs. Regardless of the sequences included, if multiple YACs are employed and contain regions of overlapping similarity, they can be recombined within yeast host strains to produce a single construct representing the entire locus or desired portions of the locus (e.g., a region targeted with a targeting vector). YAC arms can be additionally modified with mammalian selection cassettes by retrofitting to assist in introducing the constructs into embryonic stems cells or embryos by methods known in the art and/or described herein.

DNA and amino acid sequences of exogenous ADAR gene segments for use in constructing an engineered ADAR1 locus as described herein may be obtained from published databases (e.g., GenBank, IMGT, etc.) and/or published sequences. DNA and amino acid sequences of NHP ADAR gene segments for use in constructing an engineered ADAR locus as described herein may be obtained from published databases (e.g., GenBank, IMGT, etc.) and/or published sequences.

In some embodiments, a polynucleotide, e.g., a polynucleotide encoding an ADAR1 polypeptide or a characteristic portion thereof, or an exogenous ADAR gene may be codon optimized for the host non-human animal. Codon optimized sequences are engineered sequences, and preferably encode the identical polypeptide (or a biologically active fragment of a characteristic portion of the polypeptide which has substantially the same activity as the full-length polypeptide) encoded by the non-codon optimized parent polynucleotide. One skilled in the art will recognize that due to the degeneracy of codons (e.g., the redundancy of the genetic code), multiple different three-base pair codon combinations may specify an amino acid, and that a primary polynucleotide sequence may be heavily modified while retaining the primary sequence of the encoded polypeptide.

In some certain embodiments, nucleic acid constructs containing human ADAR1 gene segments (e.g., human ADAR1 and/or characteristic portions thereof) are operably linked to a human or non-human (e.g., rodent, e.g., rat or mouse) regulatory element (e.g., as described herein). In some embodiments, a regulatory element may be a promoter. In some embodiments, a regulatory region may be an enhancer.

In some embodiments, nucleic acid constructs containing human ADAR sequences further comprise intergenic DNA that is of human and/or murine origin. In some embodiments, intergenic DNA is or comprises non-coding sequences, (e.g., non-coding human sequences, non-coding rodent sequences, non-coding non-human primate sequences, and/or combinations thereof).

Nucleic acid constructs can be prepared using methods known in the art. For example, a nucleic acid construct can be prepared as part of a larger plasmid. Such preparation allows the cloning and selection of the correct constructions in an efficient manner as is known in the art. Nucleic acid constructs containing human ADAR sequences, in whole or in part, as described herein can be located between restriction sites on the plasmid so that they can be isolated from the remaining plasmid sequences for incorporation into a desired non-human animal (e.g., rodent, e.g., rat or mouse).

Various methods employed in preparation of nucleic acid constructs (e.g., plasmids) and transformation of host organisms are known in the art. For other suitable expression systems for both prokaryotic and eukaryotic cells, as well as general recombinant procedures, see Principles of Gene Manipulation: An Introduction to Genetic Manipulation, 5th Ed., ed. By Old, R.W. and S.B. Primrose, Blackwell Science, Inc., 1994 and Molecular Cloning: A Laboratory Manual, 2nd Ed., ed., by Sambrook, J. et al., Cold Spring Harbor Laboratory Press: 1989.

Among other things, the present disclosure provides that some polynucleotides as described herein are polynucleotide constructs. Polynucleotide constructs according to the present disclosure include all those known in the art, including cosmids, plasmids (e.g., naked or contained in liposomes) and viral constructs (e.g., lentiviral, retroviral, adenoviral, and adeno-associated viral constructs) that incorporate a polynucleotide comprising a human ADAR1 gene or characteristic portion thereof. Those of skill in the art will be capable of selecting suitable constructs, as well as cells, for making any of the polynucleotides described herein. In some embodiments, a construct is a plasmid (i.e., a circular DNA molecule that can autonomously replicate inside a cell). In some embodiments, a construct can be a cosmid (e.g., pWE or sCos series).

In some embodiments, a construct is a viral construct. In some embodiments, a viral construct is a lentivirus, retrovirus, adenovirus, or adeno-associated virus construct. In some embodiments, a construct is an adeno-associated virus (AAV) construct (see, e.g., Asokan et al., Mol. Ther. 20: 699-7080, 2012). In some embodiments, a viral construct is an adenovirus construct. In some embodiments, a viral construct may also be based on or derived from an alphavirus. Alphaviruses include Sindbis (and VEEV) virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O′nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, the genome of such viruses encode nonstructural (e.g., replicon) and structural proteins (e.g., capsid and envelope) that can be translated in the cytoplasm of the host cell. Ross River virus, Sindbis virus, Semliki Forest virus (SFV), and Venezuelan equine encephalitis virus (VEEV) have all been used to develop viral constructs for coding sequence delivery. Pseudotyped viruses may be formed by combining alphaviral envelope glycoproteins and retroviral capsids. Examples of alphaviral constructs can be found, e.g., in U.S. Publication Nos. 20150050243, 20090305344, and 20060177819.

Constructs provided herein can be of different sizes. In some embodiments, a construct is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some embodiments, a construct is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In some embodiments, a construct is a viral construct and can have a total number of nucleotides of up to 10 kb. In some embodiments, a viral construct can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some embodiments, a construct is a lentivirus construct and can have a total number of nucleotides of up to 8 kb. In some examples, a lentivirus construct can have a total number of nucleotides of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 8kb, about 6 kb to about 7 kb, or about 7 kb to about 8 kb

In some embodiments, a construct is an adenovirus construct and can have a total number of nucleotides of up to 8 kb. In some embodiments, an adenovirus construct can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 kb to about 8 kb.

Any of the constructs described herein can further include a control sequence, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or additional untranslated regions which may house pre- or post-transcriptional regulatory and/or control elements. In some embodiments, a promoter can be a native promoter, a constitutive promoter, an inducible promoter, and/or a tissue-specific promoter. Non-limiting examples of control sequences are described herein.

### Exemplary ADAR1 Sequence Embodiments

In certain embodiments, an ADAR1 polynucleotide or a human ADAR1 gene incorporated into a non-human animal (e.g., a rodent, e.g., a rat or mouse) is represented by or comprises a sequence encoding a human ADAR1 or genomic locus, or a characteristic portion thereof. In certain embodiments, an ADAR1 polynucleotide or a human ADAR1 gene comprises, or consists of, a nucleotide sequence that has significant portions (e.g., approximately 50%, 55%, 60%, 65%, 70%, 75%, 80%, 85%, 90%, 95%, 98%, 99%, and/or 100%) of a complete genomic polynucleotide or locus (e.g., represented by SEQ ID NO: 1), or a portion thereof, which may be recombined in any appropriate manner.

SEQ ID NO: 1 - Adenosine Deaminase Acting on RNA 1 (ADAR1) - Genomic Sequence

GAACCGGAGCCATCTTGGGCCCGGCGCGCAGACCCGCGGAGTTTCCCGTG
CCGACGCCCCGGGGCCACTTCCAGTGCGGAGTAGCGGAGGCGTGGGGGCC
TCGAGGGGCTGGCGCGGCCCAGCGGTCGGGCCAGGGTCGTGCCGCCGGCG
GGTCGGGCCGGGCAATGCCTCGCGGGCGCAATGAATCCGCGGCAGGTAAG
CCGGGCCGGCCTTGGACCTTCGCCGCCGTCTGGGTTCGTTTACAACCTCA
CAGGCTTTGTGTTGCAGTGCGTAGCGTGTGCGTCTTGTGAGTGTTAGAGT
GTGTGTGTGTGTGTCGTCTTGCCAAGCAGCATTGCTGGTTTAGGAATTTG
TGCGTCTTGTGAGTGTGTGTGTGTGGGTGTGTGTCGTCTTGCCAAGCAGC
ATTGCTGGTTTAGGAATTTGTGCGTCTTGTGAGAGTGTGTGTGTGTGTGT
GTGCGTGTGTGTGTAGTCTTGCCAAGCAGCATTGCTGGTTTAGGAATTTG
TGAATTTGTATCCTGCTCATTAATTCTGCAGAATGGAGCAGTGCGTGAAG
AGGGCTTGGGGGAAAATGCGCCCCCGTCTGAGTAGGAAGGCCTGAGCCCA
TGTCAAGGCAGACACATCGTCTCCCTTTCTGCTAGGGCCCCTTGTGGAAC
CCCCTACCCCCGCTTTAGCCCCACTTGAACAACGTTCGGACTTTGAGCAG
CGCACACTATCCTCAGCTCACCTTATCCACCTCCTGAAGGCCTTCTGGGA
GTTAAAAATGGCACTTAAGCTGTAGGAGAAAGCTTGTTAACCACTTTATA
GCTAAAAACTGGGAAAACACAAATGGCCTTCAGCAGGTTAACAGATAAAC
TGAAATACATCCACATAATGGGATACTGCTTAGTAGTGAAGAGGAAATAC
TGTTACAAGTAACAACACGGGTGACTCGCAAGTGCGTTATGCTAAGCACG
AGAAGCCAGACTCAAAAGGCTGCATACTGTATGAGTCCATTTATATGACA
TTCTGGAAAAAAAAAACCACAGTTATAGGGATGGAAAGTGGATCCGTGGG
TGCCAGGGACTGGGTATCGTGGAAGAAATTGATTGTTGAGTGGCATGAAA
AAGCTTTTTAGGGAAATAGAAATGTTCTATATCTTGATTGTGGTGGCGAT
TGTCGAAATTCATAGATTTATACACTTAAAAGGATAAATTTTACTGTATC
TAAATTATATACCTCAATTTTGTTAAGATATATATATATTTTTTTTTTTT
TAAGCACTCCTTTGAAAGGATTAAGGACGCCTAACTTGAAGGAAAAGCAT
TTCTGCACAGGTGTCAGTGTATTGCACTGTGGAACCTGTGTGGTAAAGGC
AAAGGGGGTAGTGCTTATCTCTTGATCCTAAATATGTGAGACCAGATTAA
AGTGAAATCTGGGAGGCAATGAATGTTAAATGAGTTGTTATGTAATTTGC
ATAGAGGTGATGCTGAGAGATTTAGAAAGGATCACTGTGGGTTGCTTGCT
CACTTTCTTGCTCTCCTATTCCGTAGCTTTCCAAATGGCTGTACTCAACG
GTGGCTTGGTGTTTAGGGGATTTAAGGGGGGCAAAAAGAAAGATTAATAA
TCTCCTCCTCTCCCTCTAACCCTACTGCCCTAAGATATCCTTAGCAAACT
TACATCTCCTTTCTTTTCTCTGTGTTCATTCCATTGTGCGCACACATACA
CATTCATGGATTTTCTCTTTTTGTTTAGGGAAAAAAATTATAATGTACAT
ACTATTCTACAACTTTTTGTTGTTTTATTGAACATTATATGATTCCTAAA
TTATCCCCAGGTGAATACAAATAGATATGACACATTTTAAAAAAATAAAA
TAACTGGCCGGGCGTGGTAGCTCATGCCTGTAATCCCAGCACTTTGGGAG
GCCGAGGAGGGCGGATCAGGAGATCGACACCATCCTGGCCAGCATGGTGA
AACCCCATCTCTACTAAAAATACAAAAATTAGCTGGGTGTGGTGGCGTGC
GCCTGTAATCCCAGCTACTCCGGAGGCTGAGGCAGGAGAATCACTTGAAC
CCGGGAGGCGGAGATTGCAGTGAGCTGAGATCACACTGCACTCCAGCCTG
ATTGCAGTGAGCCGAGATCATGCCACTGCACTCCAGCTTGGCAACAGAGC
GAGACTCCGTCTCACAAGAAAAAAAATAACCGTGTGAGTACTATTCCATA
GAATGAATGTTTCATAATTTAATTCTTCTATAGACAGACATTAAAATATT
TTCCAGATTTGGGCCAAGAGTAGCAGTTTAAAAAACATTTAGCTTTTAAC
TGACTCTAGCCACTTTGAAACACAATTTTTTTTTCCCAAGGTCACTCAAA
GAGCTAATAGGAGAACCCCTAAGTCCCATAATTCAGCTCTGGGAGCCAGC
ACTCACTCTGTACACACATTTGCCTCTGTCCCTAGCAATATGGTGGGCGT
GAGGGTGCAGCAAGAGGAACAAGAAAGAAATGATTGCTTGCATAGTGGCG
TCTTGTTCATGCAGTCATTAATTCAACAAATGTTTGTTGAGAATCAGCTT
TGTGCCAAGTGCTAGAGAGGTTGAGATGATTGAAGCATAGTCCTTGACCC
CCAAGAGCTCACCATGGAATCAACTGAAGCCCCTCATCAGTACTGTGTTG
GGAATATTGAGAGTGGAGAGTTGAGTATAACTTATAGGACACCTAATGTT
AATTACCTTTCAGACACTGCAATGTGTGTGTGCCATAAAAAAAAAAAAAA
TCCAGTAGCTCTGATACGAGGGAAAGTAAATGGTTTAACAGGTGCTGAGT
AGGAGAAGCTCAAGGAGAGGAAACCCCAAGGGCTGAAGAAGGTGGGAGTC
AGGAGTCTCCTGAAGCAAGTGGCATTTAAGGAGCTCTATAAGGAAAGGGT
CAGAGTTGTGATAGGTGGCTGTGGAGGGAGATGTGCCACCTGGATTGGCA
TGTAGAGGGATAGAAAGATTATAGGCCTTTGCAATGGCCCAGTAAGAGGT
AATGAGGGGCTGGAACTGGAAGAAAACACATTTAAGACACAGTACAGAGG
TGGCAGACAAGGTGGGACTTGGCAACTACCTGATGAGATCCAGGAGATGA
GGCCAGGAGGCGGGCAGCAAAGATGACGCAGGTTTCTAGCCTTAATAGGC
TAGGAGGAGAGTGATGCCATTAGCAATAAGAACTACAGGAGAAGGAGCTG
AGTTTGAGGGAACTATTAATTTGGTTCAGAATATGTGCTGTTTGAGTTAT
GGCAGGATATTTAAGTGGACAGACTGTCGACATAGTTGGAAATTCAGATC
TTAAGCTCCCACACAAGGTAGTGGCTGGACATAGTAGATTTGAGTGCTCT
TGCTTCAGAGGGCTAGTTTAGGTTGGGGCAGTGATTAAAGCAACCTAGGA
AATAAATTATAAAGGAAGAAGAGGTCCTTAAAACCTTGGAGACTGATTAT
ATAAAGGGTGGATCCGTTAATACAGTAGCTATTAAAAAATTATAAGGGGT
GGGAAAAAGGGACAAAGAAGAAAAAAGAGGTGAAAGACCTTTGCTGTGTC
ACCAAACCCTGGGAGGAGAATTTTTTAAAAGAAGAGTACTCAATCCACAG
TGAACTAAGGCATGTTTGTTAAACACAATTGACCACCACACAGCGAAGAC
CCAAATGAGGTTCAAGAGAAGGAATTTTTATGGACCCTGTTAGCACAAGT
CAAGGTCCTTCTCCAGTACCACTGGGAAGCTTTGGAGAAGAAAAGGGGGA
CAGTGGGCCTTGGGTGGAGAAGGGAACTGACCATGAGATCCAGGTGGGGT
GAGGAGGTGTGTGAAGTCAGAGTGGGGAAGAATCAGGGTGGCTTACTGGC
AGCTTCACCGGGGTCATGCGAGGAGCAGGTTCCACCAGAGTAAGGAGTGA
AGTTGTAGAAACACAGGGAAGTCACCATCAGAAAAGAGCAGGAGTCAAGA
AACTATGGCCCACAGGTACAAACTACCTGTTTTTATAAATAAAGCTTCAT
GGTAAATGAATTGTAAATAAAGTTTTGCATATTGTGTGAGGCTGTTTTTG
TGCTACAGTGGCAGAGCTGTCTGGCCCTTTATAGAAAAAGTTTATCAGCC
ACTGGAAAAGAGTTGCAGGATTTGAGGTCTTGGTGTGATGGCCTAGGTTA
GAGTCATAGTGAGATTGAAGGAGTAGCTAGACAAGCCAATTGTGTGGCAG
AAAGGTAGGGATGGAGATCACTGGGTTAAGGATCCTTGTAGCCAGGACAC
CATGAGAGTGATTGACAAGAATGCTGAAATCCCCTAAGTGTGTCAGGATG
TGGAAGAGTAGAAGGCTAGAGTTATGGAAGAAAGGGTCCCACCTCTACCT
GTGCAGCCTCCAGGAGAGTCTGAGGAGGGGGCGGTGAGTGTGAGTAAATG
TTGTCAGAATCCCTCCTCCCAGTCTACAAGCCCCAGGGAAAGGAAGCAAG
GTGTTTACTGACACCCCCAGGCTTATAAGTACTTCCTGGCTCCATCACCC
TCCAGTGAACAGCCCTGGGGAGAAGACAGTACTGGTTTGCAGGTGGGTGG
GTGGGGAAAGGGGTCACAGGTGCTTGGTGTTCTGGTAAATGTGCATATGA
GAACATGGGGTTGCTTGTGCCCTGTCCGTCAGGGTTCAGAAGAACGTGCA
GTGGAAGCAGCTATGGGGAAGTAGCTAGGGAAGGTAGGACTGGTCTGAGG
TGGTGAGGAGCAGATGCTGCCAGCTCCACACATCCAGGAGAGCCTGGGTG
GTTTGGGCAAAAGTCTCTGGCATCCCTTCTGAGCCTGGGTACCACACTGA
AGAGTGAGGACAGTGTGCCATTTTTATCAGGAAACCCTCCAGCTCCCTGA
AGACCAAATTCTGATCCTCCTGGGATGGCAGTGAAGAGCCACAGAGATGA
CTCTGAGGTCCCGTGGCCTTTTCCCACCTGGAGATTGTTTTCGTTACTGC
GCTGTTACAGCCTTGGAGGACTGGGGTTCAGTTTCATCCAATCACATTTC
TTCTTTTGTCATAGTCATCTAAACGATAGATCTTAGAGACAGGTGGGCAA
GGGGTGCACTGGTGAGCCTGACTTAAGGAGAGGTCATCTCGTCCCTTCCC
TAGTCCCATCTCCCTTGGTTATTGTTATTTCATGTGATTGTTCTGGTTAT
TTCAGGTTATTCTGTTTTTGTTTTCAAAACAATAACATATATTTGTTGTT
CTGATTTTAAAGGGGTAATTGTTTTGGTAACTAGAAAATTACCTTCTCAA
CTCCCTTAAATTCTGTCAAAGGGAAAAGTAAGTTAGGTTGCTGGAGAGGC
TATGCTGAGGCCTCAGAACCTCTGTATTCCTGGAAGTTCTGCGTGCTTTG
CCTCCTGCTCCCCTCTCTGTGTTCCTGTTGGCAGGCCCCTAGGCAGGATT
TAGGAGGTAGGCAAGTCACCCTAGCCAAGTCATAAGCCCATGGCTCAATT
GCCTTCTCAGCCCTTCAAGGGCTGTTCCACAGGCAGCAAAGGGAGGGGCC
TCCACAGGTTCACCACTGCAGCCCTAATTCATTTTCTTTTTCCACTGTCT
TATTCTGCAGGGGTATTCCCTCAGCGGATACTACACCCATCCATTTCAAG
GCTATGAGCACAGACAGCTCAGGTACCAGCAGCCTGGGCCAGGATCTTCC
CCCAGTAGTTTCCTGCTTAAGCAAATAGAATTTCTCAAGGGGCAGCTCCC
AGAAGCACCGGTGATTGGAAAGCAGACACCGTCACTGCCACCTTCCCTCC
CAGGACTCCGGCCAAGGTTTCCAGTACTACTTGCCTCCAGTACCAGAGGC
AGGCAAGTGGACATCAGGGGTGTCCCCAGGGGCGTGCATCTCAGAAGTCA
GGGGCTCCAGAGAGGGTTCCAGCATCCTTCACCACGTGGCAGGAGTCTGC
CACAGAGAGGTGTTGATTGCCTTTCCTCACATTTCCAGGAACTGAGTATC
TACCAAGATCAGGAACAAAGGATCTTAAAGTTCCTGGAAGAGCTTGGGGA
AGGGAAGGCCACCACAGCACATGATCTGTCTGGGAAACTTGGGACTCCGA
AGAAAGAAATCAATCGAGTTTTATACTCCCTGGCAAAGAAGGGCAAGCTA
CAGAAAGAGGCAGGAACACCCCCTTTGTGGAAAATCGCGGTCTCCACTCA
GGCTTGGAACCAGCACAGCGGAGTGGTAAGACCAGACGGTCATAGCCAAG
GAGCCCCAAACTCAGACCCGAGTTTGGAACCGGAAGACAGAAACTCCACA
TCTGTCTCAGAAGATCTTCTTGAGCCTTTTATTGCAGTCTCAGCTCAGGC
TTGGAACCAGCACAGCGGAGTGGTAAGACCAGACAGTCATAGCCAAGGAT
CCCCAAACTCAGACCCAGGTTTGGAACCTGAAGACAGCAACTCCACATCT
GCCTTGGAAGATCCTCTTGAGTTTTTAGACATGGCCGAGATCAAGGAGAA
AATCTGCGACTATCTCTTCAATGTGTCTGACTCCTCTGCCCTGAATTTGG
CTAAAAATATTGGCCTTACCAAGGCCCGAGATATAAATGCTGTGCTAATT
GACATGGAAAGGCAGGGGGATGTCTATAGACAAGGGACAACCCCTCCCAT
ATGGCATTTGACAGACAAGAAGCGAGAGAGGATGCAAATCAAGAGAAATA
CGAACAGTGTTCCTGAAACCGCTCCAGCTGCAATCCCTGAGACCAAAAGA
AACGCAGAGTTCCTCACCTGTAATATACCCACATCAAATGCCTCAAATAA
CATGGTAACCACAGAAAAAGTGGAGAATGGGCAGGAACCTGTCATAAAGT
TAGAAAACAGGCAAGAGGCCAGACCAGAACCAGCAAGACTGAAACCACCT
GTTCATTACAATGGCCCCTCAAAAGCAGGGTATGTTGACTTTGAAAATGG
CCAGTGGGCCACAGATGACATCCCAGATGACTTGAATAGTATCCGCGCAG
CACCAGGTGAGTTTCGAGCCATCATGGAGATGCCCTCCTTCTACAGTCAT
GGCTTGCCACGGTGTTCACCCTACAAGAAACTGACAGAGTGCCAGCTGAA
GAACCCCATCAGCGGGCTGTTAGAATATGCCCAGTTCGCTAGTCAAACCT
GTGAGTTCAACATGATAGAGCAGAGTGGACCACCCCATGAACCTCGGTAA
GAGACCACCCAGGAACTGTACCTAGGGTTGGGGTCAGGTGCTTTTGCTCC
TGACGCAGTCTTGGCTGATTTGTGAGCAGTGCTGTTTGGTGGCGCCTATC
TTTTCCTCCTTCCCTTCTGCCTTTTAGCTAAATTCCCCTTGATTGGCCCT
TTCTCCAGATATTGAGCAGGGAATATAGACCTTGGACCAGCCAGAATCTT
GGCTGAACAAGGGGGAGGTTGACTCTGTTGGCTGTAATGAAGCTTCTTTA
GAAATGATTGGTTTTGGCCGTACGCGGTGGCTCATGCCTGTAATCCCAGC
ACTTTTTGAGGCCGAGGCAGGCATATCACGAGGTCAGGAGTTTGAGACCA
GCCTGGCCAACATGGTGAAACCCTGTCTCTACTAAAAATACAAAAATTAG
CTGGGCGTGGTGGCGTGCACCTGTAGTCCCAGCTACTCAGGAAGCTGAGA
CAGGAGAATCACTTGAACCCAGGAGGCAGAGGTTGCAGTGAACTGAGATT
GCGCCACTGCACTCCAGCCTGGGCCACAGAGCAAGACTCCATCTCAAAAA
AAAAAAAGAAAGAAATGATTGGTCTTGGGGGCCGGGGCGGTGGCTTACGA
CTGTAATCCCAGCACTTTGGGAGGCCAAGGCAGGCAGATCATGAGGTGAG
GAATTCGAGACCAGCCTGGCCAACATGGTGAAACCCCATCTCTACTAAAA
ATACAAAAATTAGCTGGGGGTGGTGGTGCTTGCCTGTAATCCCAGCTACT
CGGGAGGCTGAGGCAGGAGAATCACTTGAACCCAGGAGGTGGAGGTTGTA
GTGAGCCGAGATTGGCGCCACTGCACTCCAGCCTGGGCGACAGAGTGAGA
CTCCATCTTGGAAAAAGAAAGAAAAAAGAAAAACATGATTGATCTCCATG
CATCAATATCATGCCTGCCTCCTAAGGCAGAGGTAATGAAGACTTAATTC
CCTTCTGTAGGCCTTCCCCTCCTCCCTAAGCCGTTTTCTGAGAGAGGTGC
AGGAGCAGGTGGGTTGGGGCAGGCTGCATACACAGTGGGGGTGGGTTGTG
CTGCTAAGCAGCAGCAGGTCCACAATCCCCCCTCTGCATAGCTCCTGGGG
GGAAAGGATGGAGGAGCGTGTGCACGGCTGCCTGCCTGTTGAAGGTGGTG
GTTCTAATTTTATAAACCTCCTCTGCACAGATGGGTAGGCTAGCACTTGC
TGCCACTCCTGAGCTGTGAAGTCAGCCTTTACCTCACTCAGATAGCTGGT
CAGGCCCTGCACTGTAGGTCCTAATAGGCCAGTGGACAGATTGAGGAAAA
CAGGAGCTTCTGAAGGGCATAACAGAGAGCAAAACCACTGAAGCTGAGTG
GCTGCAGCTGCAGCCAGGGAAAGAGCCAGTAGGATGGGGGAGAATTCCAC
TGACCTTTATGTTTACCTAGCCTGGTTTCTAGGGGTGTAGATTCCTGGCT
AGGGCCCTTATTCCTTGTCTTGACTGTCTTCATGACACCAATTTGGCATT
TCAGGAGAGCGGTTAAGAAAAGGAGTTGTGTCTGTCCAAAAGCTGGCAAG
GCCAGAGCTGGATTGTTTGGGGTAGAGACTGGATGGCCGTCATTCTCTTT
TGCCTCCATCCCTCCTCCCCAGAGTTGGAGGAAAGCAGTGGATTTTGTGG
TTAGTCATTCTTTGGACTCACACTAAAAGAAACATTGGTGCCATGTTCAA
ATATATCAGAAGACCTAGGAAATAAGAAATTTGACCTACTTTTCTAAATG
AAATCCCAGACTGAGCAAAGAGCTCACCACATTTGAAAGCTTGAACAAAG
GGGGCCTAGGCTAAGTCCAGAGGCCTAGAACAAATGCTTTTTTATTTTCT
ACATAACAAGGGGAAATTCCTTGTTATGTAGAAAATAGCTGGAGACAAAT
GGTGCTATAGAGTGACTCATAACAAACTACGGTGATATAGGTCTAGGGAC
AAAAGCAGGCCACTGATAAGTGGCAGATGCCTGATCCCCCTAGGTAGTGG
GGAGTGTGAGACTGGGTTATAAGAAGCCTTCACTATCTTTTAGGACCCTC
CCTTGAGGAGGCCAGTCTACCACAATTGCTTTAGAATGAAGGTCTTTTGG
TTGCTCACAAGACTATAATGGTAATTTTTGGCTCATCATTTTTGTGTGTG
TGGGTTTTATCTTAATTCATTGTTAAAGAGATAGTGGGTTTCCCCTGAGC
TAGTTTCCTATCATCTGTGCCTATGTTTGCTTCACTGAGCTATGGGGAAA
GAGTCACTGGCTGCTTTGTTTACAAAAAGAAAGGACAGGCTGAGCCTTAA
GGAGTAGGAAGGAGTTCCTTGGCCTACCCTTCATCTCCACAAGTGAAAAG
CCCCTTAGCGTAGCAGAAAATTCCAGGTTGAAGGTCTCTTTGGAGAAGGC
AGAAGGAGTGACCTAGACTCCTGTTCACACATCTAATCACTTTCCGTCAA
GATTTAAATTCCAGGTTGTCATCAATGGCCGAGAGTTTCCCCCAGCTGAA
GCTGGAAGCAAGAAAGTGGCCAAGCAGGATGCAGCTATGAAAGCCATGAC
AATTCTGCTAGAGGAAGCCAAAGCCAAGGACAGTGGAAAATCAGAAGAAT
CATCCCACTATTCCACAGAGAAAGAATCAGAGAAGGTAGGTGTCCTCCTG
CCATCTGGGAGGATCAGTTCCCTGTCAGTGTCTGGATTGTAACTGACTCC
CTTGAGGCAATCCCAGCCTAAACAACTGACACCATCTCGGAGTCAGCTTC
CCACTCTTCCCTCTACCCTATCTCGCCAGCCCACCTTTCCTCCTCTCATA
CCCAGCTCCTCTGTCTGCTCCTGTCCCAACATAATTGGATTTACAGAATT
TCGGACTTGAAAGAGACCTGAAAGTTTATTGAGGCCAATCTCCTCTCTTG
GAAGATGCGGAATGAGAGCTCCAGCAGCCCCTTTAACTTTGGGAAGCCAA
CCCCTTGACAGGTGGTGGGAATAAAGTCCCCCATCCCCATCCCTTCTTCT
GTGATGGACTAACCAGTGTTTTCTTAGTTTTGTTTTCTTCTTGTCTCTCA
TTTTCTCTAGACTGCAGAGTCCCAGACCCCCACCCCTTCAGCCACATCCT
TCTTTTCTGGGAAGAGCCCCGTCACCACACTGCTTGAGTGTATGCACAAA
TTGGGGAACTCCTGCGAATTCCGTCTCCTGTCCAAAGAAGGCCCTGCCCA
TGAACCCAAGTATGTCCTACGTGTCCTCTGTCCAGCTGAGGTTTTCTCAG
AAAGAAAAAGAGACACATTTTCTTCCTTGCCTCCTCAGGGATGGCAGATT
GACCAATTTCTCCTGTTTCAAAATGGGGAAAGGAGGGCTCTGAGGTCCTG
GTTGCTGCTTGTGAGGCAGACAATTAGGGATTAGGAATTCAAAGGGAATT
CCTGGCCCACCCTCTATCTCCTCTATAAGCACTAGGGAGGTTCACGGCTT
GGAGGTCACTCACTGTTGGTGGGACAGAAAGGTACAGGACCTGAGAAGCT
CTCTGCCTGTGGGTCTATAGACTCACATGTTCAAGAGAAGTGTTCCTGGA
AGAGGTGAAACTAGGTTGATGCTTAAATGTTGAAAGGGGTCAGCCAGGCT
AAGAGACACTGGCTGTTCAAGGCAGAAGTGACTGCATGAGATGTGACTGC
ACAGAGGTGATGTGTTCAAGGAAATTACCAGTAGTGGAGGATGGCAGCCT
GGCAGAGGCTAGGTCAGGCTCCTCAGTCAAAACCATTTTAATCCTTCTAC
GTGCTTCATCTCCTGTCAGGTTCCAATACTGTGTTGCAGTGGGAGCCCAA
ACTTTCCCCAGTGTGAGTGCTCCCAGCAAGAAAGTGGCAAAGCAGATGGC
CGCAGAGGAAGCCATGAAGGCCCTGCATGGGGAGGCGACCAACTCCATGG
CTTCTGATAACCAGGTAGGGCGTTTTCCTACTCAAAAGATACAGGTCATT
TTTAGCAACTGAGTGGTTTAAGATTGCCAGTGACTCCCTCAACATTTCCT
GAAGTGTTTATGGCTCCTCTGTTTGATGGATTCTCCTTTAGCCTGAAGGT
ATGATCTCAGAGTCACTTGATAACTTGGAATCCATGATGCCCAACAAGGT
CAGGAAGATTGGCGAGCTCGTGAGATACCTGAACACCAACCCTGTGGGTG
GCCTTTTGGAGTACGCCCGCTCCCATGGCTTTGCTGCTGAATTCAAGTTG
GTCGACCAGTCCGGACCTCCTCACGAGCCCAAGTGAGTGTCCTAGTCCTG
GCTAATGCATGTGTCACCAGTTGGGGATGGTCTGTAACCCAGGGAAAACA
AGGGTGTGCTTTAGCTGTGTAGGACAGAAGGGGCGAGTTGAGGGAAACAA
GTCCAGCCCTGTCTCCACGGCCTCTTAGAAGACAATAGACCTGCCAAGAG
TGAATGCGTTCACTCTTCCAGTAAGCATGATCCTTTTTAATTTTTTGACT
AGTTTTAATTTTTAAAGAAATGTATAGGTACATTAAAAAATCATTCAGGC
CAGACATGGTGGCTCACGCCTGTAATCCCAGCACTTTGGGAAGTTGAGGC
AGGTAGATTACCTGAGGTCGGGAGTTCAAGACCAGCCGGACCATATGAGG
AAACCCTGTCTCTACTAAAAATACAAAAATTAGCTGGGCGTGGCAGCGGG
TGCCTGTAATCCCAGCTACTCGGGAGGCAGACAGGAGAATTGATTGAAGC
TGGGAGGCGGAGGTTGCAGTGAGCTGAGATCGCACCACTGCACTCCAGCC
TGAGCAACAGAGCAAGACTCCATCTTGAAAAAAATCATTCAAGCTGATTG
AAAAAGTAGTCATTTCAATGAAAAGTCTCATTTCCGTCTCAGACTTCTAG
TTTCCCTCCCTAGACCCATATATTACTATAGCCCTGCATTAGCAACTGGG
ATGCGGTCTGAGAAGCATGGTTTTGTTGTTGTGAGAACATCAGTGTGTAT
TTACATAAACCTAGATGGCATGGGCTCCTACACACGTACAGGCTGTATGG
TATGGCCGGTTGCTCCTAGGCTACAAACCTGTACAGCATGTAACTGTAGT
GAATACTGTGGGCAGCTGCAACACACTGGTAAGTATTGGTGTCTATCTAA
ACAGCAAAAAGATACAGTAAAAAGACAGCATAAAGGATTAAAAAAATACT
ACACCTGTATAGGGCGCCTGCCATGAATGGAGCTTGCAGGACTAGAAGTT
GCTGTGGGTGAGTCAGCCAGTGAGTGGAGAATGAATATGAGGGCCTAGGA
CATGACTGTACACTAATGTAGGCTTTGTAAACACTGTTCACTTAGGCTAA
CTACATTTATTTAAAATATTTTTCTTTAATAAATTAACCTTAGCTTACTG
TAACTTTTTTACTTTATTTTTACTTTATTTTTTACTTTATTAACTTTTTT
ATTTTTTGTTCCTTTTGTAATAATACTTAGCTTAAAACACAAACATGTTG
TACTGCTATCCAAAAATATTTCCTTTGTTTATATCCTTAATTCTATAGTC
TTTTTTCTGTTTGTAAATTTTTTTATTTTTTTAACTTTTTAAACTTTTTT
GTTGAAAGCTAAGATGAAAATACATTAGCTGTTAGCCTAGGCCTACACAG
GGTCAGGATCCTCAGTATCACTGTCTTCCACCTCCACGTCTTGTCCCACT
GTAAGGTGTTCAGGGGCAATAACATGCATGGGGGCCGTCATCTCCTATGG
TAACAGTGCTTTCTGGAATACCTGCCTGAGGCTTTTTTTAGTTACTTATT
TTTCTAGAAGTAGAAGGAGTATACGCTGAAATAATGATAAAAATATAGTA
AACACATAAGGAGCGTGTAGCCTAGATCCCTCGCATTCACAGTTCACAGT
CGAGTTCACACTCCTACAAGAATCTCATGCTGCTGCTGATCCCACAGGAG
GCGGAGCTCGGACCAGAATGCTTTCTTGCTCGCCACTCACCTCTTCCTGT
GTGCCCAGGTTCCTAACAGGTTACAGAGCCCTGCACTTGGTGATAAGAAT
TTTTCAGCTCCATCATAATCTTACGGGGCCCCTGTCATATGTGTGATTTA
TCATTGAACAAAACACTGTTATGCAGTACATGACTATACCAGTTTCTTGA
GTCCTTCTAGAAATTGCTGTGCATAACTAGCACATTTTCCCTTTTGTCTT
TTTAAACAAGTATATCTTCTGTTTTATACGTTGACTCCTTTACTTACCAG
ACTTCTTGCATAGCTGCATAGTATTTCATTGAATAGAAGCTGACAAATTC
AGTTTCTCAGAAGTAAACATTTAATAGGGACTTAGGAACAGAAATGATGT
CTTGGGTGGCTGCAAGATGGTGGATCCCTGCACTTACCCTCCAGAAAGTA
TGCTTTCTATAGAGGCTTTTTTGGTTAAACATGCAGCTGGTCACACATTA
TACTTTCTTGTGAAACTTATGAGCACTAGCTGGTGGGGAGGCTTGGTAGA
CATCTTTGTGTGGAATTATTTATGCTACCCAACACTTTGGGATGCGGGAG
TCAGATATTGGTGGTCATAGTGGTTTTGCATCAAGATGGCATCACTCTTG
CCATGCAACCGGCATGTTTTCTTAATCTCTAAGGTCTATCACAAAGTGGA
AAAGCAAGGTGTAAAGTGAGGGGAGAGGTGAAGATTGTCTGTATATGTCT
AGAATATTTTCTTGAAGGATCGAAAAAACTGGCAACTGTGTTTGCTTCTG
GGGAAGGAGGGACACTTTTATTGTACACTCTTAACTGTTTGAATTTTCAC
ATGTTTGGCATCCAACTTGTCCATTTTAAAGTCATTAAGGAAAAAAGCTT
ATAAAAATGCTATCTTGTTACTTTAAATTTGTCTCTCCTTAATAGAAGTA
ATGTCAGCCGGGCGCAGTGGCTTACGCCTATAATCCCAGCACTTTGGGAG
GCCGAGGTGGGTGGATCATTTGAGCTCAGTAATTTGAGACCAGCCTGGCC
AACATGGTAAAACCCCATCTCTACTAAAAATACAAAAAATAGCTGAGCAT
GGTGGTATGCCCCTGTATGCCAGCTACTCCGGAGGCTGAGCAGGAGAATT
ACTTGAACCCAGGAGGCGGAAGTTGCAGTGACCTGAGATTGTGCCACTGC
ACTCCAGCCTGGGTGATGGAGTGAGACTCCGTCTCAAAAAAAAAAACAAA
AAAATCAGAAGTAATGGCATTTCATTTATTCATATTTATGTGTTATTTGC
ATTTCCTTTTCTGTGAACCACCTGTTCTTGTCCTTTTCCAGTTTTCTTTC
AGATTGTTACTCTCATGGATATGTAAGAACTTTTAACATAATTTTTAAAA
GTAGCCCTTTGCCCAACATGACCCAATTTAGAACACACATCTGCCCACTG
TTTAAGCCATCTGGAAAAGGAGAGGCCCAGTCTCCCTCCAGCTGCTCTTC
TACTAATTCATATCCTTCATCTCAATAAGCACCTCCTTATCTTTAAGCAC
AGTCCTTCAGATGATAGGTTCAGTGCATTTCCTCTTTCTCTTCTGTGAAA
TCTTCTGTGAAATCCTTTCTAGGACTTCAAGTCAGCCATTCATTGAGAAT
GTAAGAACTTACTATGAACTAGATGGAAGATACAAAAAATTGCTGCCCCT
GAGTGTGTAGCAGACATATAGCCATAGCAGTATTGAAATGTGCCATTGGA
GGTGAATGCAGAGTGCTGTGGGGACACAGAGGACACTGCTGCCACTGTCT
GCCAGGGAGCTGAGGAAGGTTGCATAGAGAGGGGAAATGCTAATTGAGTA
ACTACGTTTATTAACTATCTCGTACAATAATATACTTTAGTTGCTTCTTA
ATGTGAATTATGCTGGGTCTTTTTTTCCTTTCTGTCTACTGTGGCTGGTG
ACGTTAACTAATTTTATGACTTCTTTTCTGTTTTTTTTTTTTTTTTTTTT
TGGAGATGGAGTCTCACTCTGTCACCCAGGCTGGAGTGCAGTGGCGCAGT
CTCAGCTCACTGCAACCTCCGCCTCCTGGGTTCAAACAATTCTCTTGCCT
CAGCTCCTCGAGTAGCTGGGATTACAGGCATCTGCCACCACATCTGACTA
TTTTTGTATTTTTAGTAGAGACAGAGTTTCACCATGTTGGCCAGGCTGGT
CTTCAACTCCTGACCTCAGATGATCCACCCGCCTCAGCCTCCCAAAGTGC
TGGGATTACAGGCATGAACCACCGCGACAGCCATGACCTTTAATATCTAA
ATGCTGGAGACCACTCAGAGCTCCCGATTCCTCCATCTAGTTGCTTACTT
GTCACCTCTGTTTGGATTAACATCCCTAATATGACATGTCCAAAGCAGAA
CTCTTGGGTTTTGCCCCCTTCGCTTCATTTCCCCCAAGTCTTTCCCAGCT
TAGTAAGTGATACCACTGTCTACCCAGTGGCTCAAGTCAGAAACCTGAGG
ATCATCTTTTCCTCTATCTCCTTTACCTCCTTCGTCAACTCATCCTTAAG
TCCTATTGGTTATACTTAGAATTTCTATCCCAAATCCCTTCCTCTTCTTT
CCACCTCCAGCGTCAGCACTCTAATCCAAGCCACTGTCTCATCTAGACTG
TCACAATAGCCTCCTAACTGATCTTTATACTTTATTTACTCAACACTTAT
TTATGGAGTATCTTCTATTTTTTTTAGAACAGGAACCAAACATTAACCAA
TAGTCACCTAAAGAAATGTAAGATCTCATATTTGATAAGTATTCCAAAGG
GCTGGAGTTGCAGCGAGAATCAATTATAGCACAGTTTGACTTAGGAAGGA
GAGGGGGAAGGGCTTCCCCAAGAAGTACTGCCTGAGCTGAGACTTTAAGG
ATGATGAAGAGAGAAAGACTGAGGGTTCTAGGCGAAGGACACTGTTAAAT
CCTGTGGTGGGAGAAGCGCAGAGCTGGCAAAGAGGCTGAGAGGAGGCACT
TATGGCAATAGCCCCATTTGGCTAGTGGCTACCATATTGGATAGCACAGC
TCTAAAGGATTCTAGTTGCATAATTAACAAACTTTATCATATATGTAAAT
ATATCAGTATAAAACTTTGCACGTAAGAGTTCATATTCTTTTCAAACACA
TGGGGCAGTCATGAAATTCTCCTGGATCCCTGAGGTAAAGATACTACACA
ATCTGTCCCTCCTGACCGCCTTCACCATCTTCTTTTCATGCCACTGTCCC
TCTCATTCTGAACTTAAGCCCACTGGCCTGCACGGGCCTGCCAGCATGGA
CAAAGAAAAGAGTGAAGGCATTTAGTTGCTTCGGATCATCAAGGAACAGT
GAGAAACTCCATGTGGTTGAACATAAACGTTGATTCCTTTTACTGCCTGA
GCTGCTAAGTCTTGAAACTGAAGCCTCTGTTATATGCAGTTAGAGTATCC
AAAGCTGGGTTCTCAGCCAGTAGATTAGGATCTGGTTGAAAGCTAGGATT
CTTGATGCCTTGTCTGGTACATTTCCAAGCTGTTTTGCTCCACCATGCTC
TTCCCTCTGTGTTTGGAAATTACCAGCCGGAGCGGTAGAAGCCACCTCCC
TGCTCCACTTGGGCACTTTATTGTCAGCGCAGCTCATGCGCCCAAGATCC
ATTCCTTACCCCAGAGTTAGCTTAAGCCTGCCAGGAGTCAAAAAAACAAC
TCCCAGCAAAACCTGGGTCCCTGGCCTAAGGCCGACCTTGACCTAATGAC
CTCTCCAGGTCAAGTCCCCTTGAGCTAATAGGAAGTAAATGAGGGAATGG
AGTTGGGCGCTTTAAAAATCAGACAGTGTATGAAGTAACAGTGACTTGTG
GGGAGAAAGGTAGTCCCTTCTGATTTCTGCTTAAGAAGAGAGTCCAAGTT
GGTGCTAGCATTTCGTTAAGCCTTCCTCATACTTCATAATCTTCCTTTAT
AGTAAGGACAGGCTCAGAGCCTTTGCAGGTAACAGTATCTAGTCAGAATT
TTATAACATGATTTTATCATATTCTGTTTTACAGCCACCTTCAATATGTG
GCAAGTGATACTGGTTTTCCATTTACAGTAGTGAAACAAAGTTTCCTATT
TCAGTGTGTTTACATTTTTATCTCTTTTTTCATACCTAGTATTACGGATA
TTTACATTTTTAGAGTCTGTTTGTTTATTTTAGAGATGGGGATCTTGCTG
TCTCGCGCAGGCTGGAGTGCAGTGATATGGTCATAGCTCACTGCAGCCTT
GACCTCCAGGGCTCAAGTGATCCTCTCACCCCAGCCTCCCAAGTAGCTGA
GACTATAGGCACAGACCACCGTGCCCAGCTAAATTTTTTTTTTTTTTTTT
AAGAGACAGGGGTCTCGCTATGTTGCCTGGGGGATTACTATGTTGCCCAG
GCTGGTCTTGAACTCCTGGCCTCAAGTGATCTCCCACCTTAGCTCCTCAA
GTTGCTGGGATTACAGGTGTGAGCCACAATGGCTGGCTCTAAGAAGTCAA
TTTAGAGAAAAATATTAATTAATAGTCCAAGTAATACCTGGATGTGGCAC
AAATCATGTGGTGATTCATGAATGACCAAAATCTAGGAARTGCTGCCTTA
GAAACAGCTTCTGTTTGACGGGTCCATATGTTTGCAAGACTGGCCACATC
TTCAGCAAAACTAACCCTTCCTTGGAACAATGTCAGGGTCTGGCACTTGT
CTTCATTCTCTGTTTCTCATCCCAAAGGTTCGTTTACCAAGCAAAAGTTG
GGGGTCGCTGGTTCCCAGCCGTCTGCGCACACAGCAAGAAGCAAGGCAAG
CAGGAAGCAGCAGATGCGGCTCTCCGTGTCTTGATTGGGGAGAACGAGAA
GGCAGAACGCATGGGTTTCACAGAGGTAACCCCAGTGACAGGGGCCAGTC
TCAGAAGAACTATGCTCCTCCTCTCAAGGTCCCCAGAAGCACAGCCAAAG
ACAGTTAAGACGTCTACTTTTGGTGCCTTTTTTGGGGCGGGGGGGTCCTC
CTAACTCCTAAGTGGAGGTGGCTCTTGCTGTCATGCGAGTTATTCCTAGG
CTTTACTCTTAGCCTCGAGAGAGCAGTAACTGGGACACTAGATGTAAGAA
GGAAAAGATGACTCACACGACAAGTAGAGCTTGATCTCCCTGCCCACGGT
GAATATGGTGGACACAGCCTCAGCTTTGTGGTGCTGACACAGCCTCTTTT
CCCCACAGCTCCCTCTCACTGGCAGCACCTTCCATGACCAGATAGCCATG
CTGAGCCACCGGTGCTTCAACACTCTGACTAACAGCTTCCAGCCCTCCTT
GCTCGGCCGCAAGATTCTGGCCGCCATCATTATGAAAAAAGACTCTGAGG
ACATGGGTGTCGTCGTCAGCTTGGGAACAGGTGAGTGAGGCTCTGAGACA
TGCCGCCTCCCATGGCGCCTGAAAGCGGGTGCCTCTCATCCTCCCCTGGA
GTCCATGCATGTAAGTCCAAGGCAGGGAGAAGAGACTTCATTTTAGCTAC
AGTCAATTCAGAGTGAGGAATGAGTTCTTAGTTCCTAGAGGAGAGAATAT
GGGAGTCTAGGATCTGAGAAACTGAGGCTGTTTCTGCCTTGAAGCTTTCA
GAACAAATAGCCTTCATCCTGTTTTCCATCGGTTTCCTTCCATTATTCTA
TTTCTGTTTTAAACACCTTCCTAGGGAATCGCTGTGTGAAAGGAGATTCT
CTCAGCCTAAAAGGAGAAACTGTCAATGACTGCCATGCAGAAATAATCTC
CCGGAGAGGCTTCATCAGGTGAGCGAGGTCAGAGCTGTGGCCCGGCTGCC
CGGCTGTGGAGAGCTCCAGTTCCCTGCCCCACATGGCTCTGACACGGCCT
CTGAATCCCCCTCAGACAGACGGGTCATGATGTGGCAGTGGCAGCCTTTG
CTTTTCACCCGTCCATTTGAACCTGTCTGATGGAATCCATCCCCTCTGTG
AGCTGAGCTGCCTCCCACTGCTCGGCCTGTTTTTAAATGCTGTCCTTTTT
TCTGCTAACTCTGCTGCTTCATGTTCTTTTCTAAAAACACAAAATGACCT
TTTAGTCCTCAGGGCCTTGAGGATGAGGCAGCTTTCCATTTCCGTTTGAG
GACCTACACAACCTTGATGCCCCTGCCAGCTTTCTCCTCTAGCTCACCTT
TTCTTTAATTTATGAAGGGAGAGACTTAGAAAGGAGCAACAGCTTCCTGT
AGTCCTTGAATCAGTTTGCTCTGCTCTAGAATCCCTGTAGCCGCCATAGC
GAGGAGCCCTCAGCAGAAATGAAGGAGACCCAAAAGGCTAACTATGCTTT
ATGAAATGCTGAGGTCTCCCCTGGAGAATTTCCACCTGATAAACTGTGAA
ACGTCTGCAACATTGAGACTTTTCCTTACTTTCTCATTTGGAGGTCAGAT
TATAGAAACAACTGCTTTTCCCAGAATTGAACCTGCCTTCCTAACCAGAC
TTTCTTTTTGTAGGTTTCTCTACAGTGAGTTAATGAAATACAACTCCCAG
ACTGCGAAGGATAGTATATTTGAACCTGCTAAGGGAGGAGAAAAGCTCCA
AATAAAAAAGACTGTGTCATTCCATCTGTATATCAGGTCTGTACAGTTCC
TGTTGCTGCCAGGGTGGGCCCTGCCAGGCTGTTAGAATTGGGTATCCAAA
TGCTCTCCTGGCCTGTAAATCGAACCTGATACAATAAGCCACACTCCACT
GTGGGTTTGAGGTCCATATTCAGGTGTAGATGACTCACATGTACTGCTGT
CCACCTCCAGTCTCCCATGGTAGGCCTTAGAAAACATCCCTTGCTTCTGT
CACATCTGACTGTTTTGGAGCCCCACGAAATTGCAGATTTCCCACAGGTG
AGTTTTAACAGCCACCCCTGTTTTTCAGCACTGCTCCGTGTGGAGATGGC
GCCCTCTTTGACAAGTCCTGCAGCGACCGTGCTATGGAAAGCACAGAATC
CCGCCACTACCCTGTCTTCGAGAATCCCAAACAAGGAAAGCTCCGCACCA
AGGTGGAGAACGGTGAGTGATACATGCCCCCGCCTCCTTTCCTCAAAAGG
CTCTGCAAGGTCCAGGGACCCCAAGTCTCTACAAGGCTGCTAGGATTTTA
CCATTAGTCACTGGGCACAGAGGTGCTGTTTACAGGAAAGGGAAGACCTG
GGTCAGGGAGCTGTGTGGTAAGATCAGGGTTCTATTTTGAATGTGTTAGT
TTGGGAGATCTGGGAGATCCCCAAGTCAAATAGGAGGTGGGGGTTCCCAT
GTGGAGCTCAGAGGAGGGAGCTTGGCTGGAAATAGAAATATGGGAGTCAT
CCCCCTATAGGGTCTTCATGGCCATGAGAATGCATAGGATTACCTCAAGA
AAGCGGGGAGGAAATGAAGAGTGCGGCACAACCAAGCCCTGAGGAGTTGA
CAGATGAGGATGCCAAATGCTGGGGTCCCCTCCTGTCTAGCTGGCAGTTG
ACTCTGCCTTGTCCACTGGCTCCTTCTCTCCTATCCTCTCCTGTCTCCTT
ACTGTCTCTTCGCATCCACTCCATTGCGTTCAGGCCACGTCAGCAGTCAT
CATGGTGGTCCTGAAACCTTGCTAAATACCCTAAAGTATAGACACAGTTA
CCATGGAGCCGGTGCTCCACTCCTAGGTATATGCTGCAGAGAGATGGAGA
TCTGTGTCCACACGGAAACTAATATGTGAATGTTCATGGCAGCATTACTC
ACAAGAGCCAAAAAAGTGGAAACAACCCAGACGTCCATCAGCTGATGGAT
TCATAAATAAAACATCAAATATATCCATAAATTGAATATTATTTTGGCCA
TAAAAAGAAGTGAAGTGCTGATACATGCTTACAATATGGATGCACTTGAA
AACTTGATGCCAAATGAAAAAAGCCAGTCACAAAAGATCACATATTGTAT
GATTCCATTTATATGAAATGTCCAGAATAGACAAATCCATAGAGACAGAA
AGTAGATGAGTGGTTGCCAGGGCCAGGAGTGGGAGAGTTGGAGAGATGAG
GAGTGACTGCGCCAATGGGTAGAAGGTTTCTTTTTGGAGCGATGAAATGT
TCTAAAATTGACTGTGGTGACAGTTGCAGAACTCTGTGAATATACTAAAA
ATGACTGAATTGTATGCTTTAAATGGGTGAACTGTATGGCATATGAACTA
TATCTCAGTAAAGCATTTTTTTTTGTTTTTTTTTAAACCCGATAGTGGTT
TCCCACTGCACTGCATATACAAGCAAAACCTGCTGTGATCACCCGGCCTC
CTCTCATGCCACTTTCCCCATCCCTCGCATATGCTCTGTCCACACTGGCT
CTCTGTCAGGCGCCTGAACAGCCAATCCGCTGGCTGCCTTGGGGCCTTTG
CTTTTGTCCTGTGTGCCTGAAACACTTACTCCAGCTGTCCTCCTCCATGT
CTGGGCTCCCCTGCTGCTGCTGTCCCAGGAGGTGTCCCCTGTGGTCCTCC
GTCATAGCTGCCTCAGAGCCTTCAGAGCACTGTCAGCATCTGGAATTCTT
CCGTATGTACTGGCTACTGGTTTAGTGTCTATTCTCTTCCCTTTCAATCT
CCTCACCACCATAGATGTTTTAAGAGCACAAAGATCTTACTTGGTTTTGC
TCACCGTTCTTTCCCAGCACCAAGCATAGTGCCTGGCGCATAGCAGGGGC
TGTGAAATATGTGAAGAATGAATGAATGTAGCCTGTGGCCCAAGCTTAAG
GAGGATAGAAACCACGCCAGGGAGTGGTTTGGTCCATTGGCGCCTGTGGG
TCTGACCCAAGTCTCTCACACAGGAGAAGGCACAATCCCTGTGGAATCCA
GTGACATTGTGCCTACGTGGGATGGCATTCGGCTCGGGGAGAGACTCCGT
ACCATGTCCTGTAGTGACAAAATCCTACGCTGGAACGTGCTGGGCCTGCA
AGGGGCACTGTTGACCCACTTCCTGCAGCCCATTTATCTCAAATCTGTCA
CATTGGGTAAGGGGCCTGCCTTGGGATCTGGAACTGGTCTGTCCTTCTTG
TGCCCAGATCCCAAACTGCATGCTTTATTGCCAGGTGTTTTGTCTCCCTT
ATCAAAGTGAGCATGATTCACTCCTCAGTAATTGATTGAGTGTCCAGTCT
GCTGTGGTAGGAAGATCCTGGTAGCCCCAGTCAGAAGGTGCTTCCTAACA
AGGCAGCTGTTTCTCTTTCTTGACAACTATATCTTGTACCTCCAAAATCC
CCACATGCTTCTGCCTCTTAACAGCATTTGGTGCAAACACAGGTATATAT
GTTTCTCTTTTTTGTACTCAGGTTACCTTTTCAGCCAAGGGCATCTGACC
CGTGCTATTTGCTGTCGTGTGACAAGAGATGGGAGTGCATTTGAGGATGG
ACTACGACATCCCTTTATTGTCAACCACCCCAAGGTGCTATAACCCCCTT
CTATTTTCCCTGACATTTTCCTCCTTTTCAAGCAGTCATGTAAACAGAGG
AAAAATGTACACTGTGGGCAAGGGGAACATTGCCCACAGTGGTAGCCCAC
AAGGGAACATTGCCCACAGTGGCCCACCACCAACATTGGTTGGTCTCCCA
AGAACTTAAACTTTCTTCCTTTTGGATGCCAAGGGCTTTTCTTCTCTTAG
TCTGGAATTAATCTGAATCGAGGTGGAGTTAGTATGTCTAGAGGGTGCTC
AGTCTTAGCCAAACAGAACCCTAAATACAGGGGAAAGATCATGACCCCAC
ACTTCCTCTCTCCTATGAGTCTTGAGTCCCTGCTTCAGAATCTTATTCCT
GAAAGGTTTCCATCTTTCTCCCGTTGCTTCTGGGATTCCTAGGTTGGCAG
AGTCAGCATATATGATTCCAAAAGGCAATCCGGGAAGACTAAGGAGACAA
GCGTCAACTGGTGTCTGGCTGATGGCTATGACCTGGAGATCCTGGACGGT
ACCAGAGGCACTGTGGATGGGTAAGGAGACAGGAGAGCGCAGTGAGGACC
AAGCCTCTGCCCTGACTTGCAAGGGTGCATCATACCTCTGCAGTCTCAGG
GCTTGAGAGCCGCCTCCCCTCCCACGGTGTCTCCACTGTGAGCTCCTTAT
CTTACAGGTCCCAGGTGAATAATGAGTGCTTTTGTTTCTCTAGGCCACGG
AATGAATTGTCCCGGGTCTCCAAAAAGAACATTTTTCTTCTATTTAAGAA
GCTCTGCTCCTTCCGTTACCGCAGGGATCTACTGAGACTCTCCTATGGTG
AGGCCAAGAAAGCTGCCCGTGACTACGAGACGGCCAAGAACTACTTCAAA
AAAGGCCTGAAGGATATGGGCTATGGGAACTGGATTAGCAAACCCCAGGA
GGAAAAGAACTTTTATCTCTGCCCAGTATAGTATGCTCCAGTGACAGATG
GATTAGGGTGTGTCATACTAGGGTGTGAGAGAGGTAGGTCGTAGCATTCC
TCATCACATGGTCAGGGGATTTTTTTTTCTCCTTTTTTTTTCTTTTTAAG
CCATAATTGGTGATACTGAAAACTTTGGGTTCCCATTTATCCTGCTTTCT
TTGGGATTGCTAGGCAAGGTCTGGCCAGGCCCCCCTTTTTTCCCCCAAGT
GAAGAGGCAGAAACCTAAGAAGTTATCTTTTCTTTCTACCCAAAGCATAC
ATAGTCACTGAGCACCTGCGGTCCATTTCCTCTTAAAAGTTTTGTTTTGA
TTTGTTTCCATTTCCTTTCCCTTTGTGTTTGCTACACTGACCTCTTGCGG
TCTTGATTAGGTTTCAGTCAACTCTGGATCATGTCAGGGACTGATAATTT
CATTTGTGGATTACGCAGACCCCTCTACTTCCCCTCTTTCCCTTCTGAGA
TTCTTTCCTTGTGATCTGAATGTCTCCTTTTCCCCCTCAGAGGGCAAAGA
GGTGAACATAAAGGATTTGGTGAAACATTTGTAAGGGTAGGAGTTGAAAA
CTGCAGTTCCCAGTGCCACGGAAGTGTGATTGGAGCCTGCAGATAATGCC
CAGCCATCCTCCCATCCTGCACTTTAGCCAGCTGCAGGGCGGGCAAGGCA
AGGAAAGCTGCTTCCCTGGAAGTGTATCACTTTCTCCGGCAGCTGGGAAG
TCTAGAACCAGCCAGACTGGGTTAAGGGAGCTGCTCAAGCAATAGCAGAG
GTTTCACCCGGCAGGATGACACAGACCACTTCCCAGGGAGCACGGGCATG
CCTTGGAATATTGCCAAGCTTCCAGCTGCCTCTTCTCCTAAAGCATTCCT
AGGAATATTTTCCCCGCCAATGCTGGGCGTACACCCTAGCCAACGGGACA
AATCCTAGAGGGTATAAAATCATCTCTGCTCAGATAATCATGACTTAGCA
AGAATAAGGGCAAAAAATCCTGTTGGCTTAACGTCACTGTTCCACCCGGT
GTAATATCTCTCATGACAGTGACACCAAGGGAAGTTGACTAAGTCACATG
TAAATTAGGAGTGTTTTAAAGAATGCCATAGATGTTGATTCTTAACTGCT
ACAGATAACCTGTAATTGAGCAGATTTAAAATTCAGGCATACTTTTCCAT
TTATCCAAGTGCTTTCATTTTTCCAGATGGCTTCAGAAGTAGGCTCGTGG
GCAGGGCGCAGACCTGATCTTTATAGGGTTGACATAGAAAGCAGTAGTTG
TGGGTGAAAGGGCAGGTTGTCTTCAAACTCTGTGAGGTAGAATCCTTTGT
CTATACCTCCATGAACATTGACTCGTGTGTTCAGAGCCTTTGGCCTCTCT
GTGGAGTCTGGCTCTCTGGCTCCTGTGCATTCTTTGAATAGTCACTCGTA
AAAACTGTCAGTGCTTGAAACTGTTTCCTTTACTCATGTTGAAGGGACTT
TGTTGGCTTTTAGAGTGTTGGTCATGACTCCAAGAGCAGAGCAGGGAAGA
GCCCAAGCATAGACTTGGTGCCGTGGTGATGGCTGCAGTCCAGTTTTGTG
ATGCTGCTTTTACGTGTCCCTCGATAACAGTCAGCTAGACACACTCAGGA
GGACTACTGAGGCTCTGCGACCTTCAGGAGCTGAGCCTGCCTCTCTCCTT
TAGATGACAGACCTTCATCTGGGAACGTGCTGAGCCAGCACCCTCAGATG
ATTTCCCTCCAAACTGCTGACTAGGTCATCCTCTGTCTGGTAGAGACATT
CACATCTTTGCTTTTATTCTATGCTCTCTGTACTTTTGACCAAAAATTGA
CCAAAGTAAGAAAATGCAAGTTCTAAAAATAGACTAAGGATGCCTTTGCA
GAACACCAAAGCATCCCAAGGAACTGGTAGGGAAGTGGCGCCTGTCTCCT
GGAGTGGAAGAGGCCTGCTCCCTGGCTCTGGGTCTGCTGGGGGCACAGTA
AATCAGTCTTGGCACCCACATCCAGGGCAGAGAGGTCTGTGGTTCTCAGC
ATCAGAAGGCAGCGCAGCCCCTCTCCTCTTCAGGCTACAGGGTTGTCACC
TGCTGAGTCCTCAGGTTGTTTGGCCTCTCTGGTCCATCTTGGGCATTAGG
TTCTCCAGCAGAGCTCTGGCCAGCTGCCTCTTCTTTAACTGGGAACACAG
GCTCTCACAAGATCAGAACCCCCACTCACCCCCAAGATCTTATCTAGCAA
GCCTGTAGTATTCAGTTTCTGTTGTAGGAAGAGAGCGAGGCATCCCTGAA
TTCCACGCATCTGCTGGAAACGAGCCGTGTCAGATCGCACATCCCTGCGC
CCCCATGCCCCTCTGAGTCACACAGGACAGAGGAGGCAGAGCTTCTGCCC
ACTGTTATCTTCACTTTCTTTGTCCAGTCTTTTGTTTTTAATAAGCAGTG
ACCCTCCCTACTCTTCTTTTTAATGATTTTTGTAGTTGATTTGTCTGAAC
TGTGGCTACTGTGCATTCCTTGAATAATCACTTGTAAAAATTGTCAGTGC
TTGAAGCTGTTTCCTTTACTCACATTGAAGGGACTTCGTTGGTTTTTTGG
AGTCTTGGTTGTGACTCCAAGAGCAGAGTGAGGAAGACCCCCAAGCATAG
ACTCGGGTACTGTGATGATGGCTGCAGTCCAGTTTTATGATTCTGCTTTT
ATGTGTCCCTTGATAACAGTGACTTAACAATATACATTCCTCATAAATAA
AAAAAAAACAAGAATCTGAATTCTTAGAAA

In certain embodiments, an ADAR1 polynucleotide or a human ADAR1 gene incorporated into a non-human animal (e.g., a rodent, e.g., a rat or mouse) is represented by or comprises a sequence encoding a human ADAR1 transcript variant 1 or a characteristic portion thereof. In certain embodiments, an ADAR1 polynucleotide or a human ADAR1 gene comprises, or consists of, a nucleotide sequence that is the same as, differs by no more than 1, 2, 3, 4, 5, 6, 7, 8, 9, or 10 nucleotides from, or has at least 90%, 92%, 94%, 96%, 98%, or 99% homology with, the nucleotide sequence of SEQ ID NO: 2, SEQ ID NO: 3, or SEQ ID NO: 4 or characteristic portion thereof. In some embodiments, an ADAR1 polynucleotide or a human ADAR1 gene comprises, or consists of, a nucleotide sequence that is the same as the nucleotide sequence SEQ ID NO: 2, SEQ ID NO: 3, or SEQ ID NO: 4 or a characteristic portion thereof. In certain embodiments, an encoded human ADAR1 Amino Acid sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the Amino Acid sequence of SEQ ID NO: 5 or SEQ ID NO: 6. In certain embodiments, amino acid sequence of an encoded and/or expressed ADAR1 polypeptide or a characteristic portion thereof, is or comprises a sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the amino acid sequence of SEQ ID NO: 5 or SEQ ID NO: 6. In certain embodiments, the amino acid sequence of an encoded and/or expressed ADAR1 polypeptide or a characteristic portion thereof, is or comprises SEQ ID NO: 5 or SEQ ID NO: 6.

SEQ ID NO: 2 - Human ADAR1 - Transcript Variant 1 cDNA Sequence

GAACCGGAGCCATCTTGGGCCCGGCGCGCAGACCCGCGGAGTTTCCCGTG
CCGACGCCCCGGGGCCACTTCCAGTGCGGAGTAGCGGAGGCGTGGGGGCC
TCGAGGGGCTGGCGCGGCCCAGCGGTCGGGCCAGGGTCGTGCCGCCGGCG
GGTCGGGCCGGGCAATGCCTCGCGGGCGCAATGAATCCGCGGCAGGGGTA
TTCCCTCAGCGGATACTACACCCATCCATTTCAAGGCTATGAGCACAGAC
AGCTCAGGTACCAGCAGCCTGGGCCAGGATCTTCCCCCAGTAGTTTCCTG
CTTAAGCAAATAGAATTTCTCAAGGGGCAGCTCCCAGAAGCACCGGTGAT
TGGAAAGCAGACACCGTCACTGCCACCTTCCCTCCCAGGACTCCGGCCAA
GGTTTCCAGTACTACTTGCCTCCAGTACCAGAGGCAGGCAAGTGGACATC
AGGGGTGTCCCCAGGGGCGTGCATCTCAGAAGTCAGGGGCTCCAGAGAGG
GTTCCAGCATCCTTCACCACGTGGCAGGAGTCTGCCACAGAGAGGTGTTG
ATTGCCTTTCCTCACATTTCCAGGAACTGAGTATCTACCAAGATCAGGAA
CAAAGGATCTTAAAGTTCCTGGAAGAGCTTGGGGAAGGGAAGGCCACCAC
AGCACATGATCTGTCTGGGAAACTTGGGACTCCGAAGAAAGAAATCAATC
GAGTTTTATACTCCCTGGCAAAGAAGGGCAAGCTACAGAAAGAGGCAGGA
ACACCCCCTTTGTGGAAAATCGCGGTCTCCACTCAGGCTTGGAACCAGCA
CAGCGGAGTGGTAAGACCAGACGGTCATAGCCAAGGAGCCCCAAACTCAG
ACCCGAGTTTGGAACCGGAAGACAGAAACTCCACATCTGTCTCAGAAGAT
CTTCTTGAGCCTTTTATTGCAGTCTCAGCTCAGGCTTGGAACCAGCACAG
CGGAGTGGTAAGACCAGACAGTCATAGCCAAGGATCCCCAAACTCAGACC
CAGGTTTGGAACCTGAAGACAGCAACTCCACATCTGCCTTGGAAGATCCT
CTTGAGTTTTTAGACATGGCCGAGATCAAGGAGAAAATCTGCGACTATCT
CTTCAATGTGTCTGACTCCTCTGCCCTGAATTTGGCTAAAAATATTGGCC
TTACCAAGGCCCGAGATATAAATGCTGTGCTAATTGACATGGAAAGGCAG
GGGGATGTCTATAGACAAGGGACAACCCCTCCCATATGGCATTTGACAGA
CAAGAAGCGAGAGAGGATGCAAATCAAGAGAAATACGAACAGTGTTCCTG
AAACCGCTCCAGCTGCAATCCCTGAGACCAAAAGAAACGCAGAGTTCCTC
ACCTGTAATATACCCACATCAAATGCCTCAAATAACATGGTAACCACAGA
AAAAGTGGAGAATGGGCAGGAACCTGTCATAAAGTTAGAAAACAGGCAAG
AGGCCAGACCAGAACCAGCAAGACTGAAACCACCTGTTCATTACAATGGC
CCCTCAAAAGCAGGGTATGTTGACTTTGAAAATGGCCAGTGGGCCACAGA
TGACATCCCAGATGACTTGAATAGTATCCGCGCAGCACCAGGTGAGTTTC
GAGCCATCATGGAGATGCCCTCCTTCTACAGTCATGGCTTGCCACGGTGT
TCACCCTACAAGAAACTGACAGAGTGCCAGCTGAAGAACCCCATCAGCGG
GCTGTTAGAATATGCCCAGTTCGCTAGTCAAACCTGTGAGTTCAACATGA
TAGAGCAGAGTGGACCACCCCATGAACCTCGATTTAAATTCCAGGTTGTC
ATCAATGGCCGAGAGTTTCCCCCAGCTGAAGCTGGAAGCAAGAAAGTGGC
CAAGCAGGATGCAGCTATGAAAGCCATGACAATTCTGCTAGAGGAAGCCA
AAGCCAAGGACAGTGGAAAATCAGAAGAATCATCCCACTATTCCACAGAG
AAAGAATCAGAGAAGACTGCAGAGTCCCAGACCCCCACCCCTTCAGCCAC
ATCCTTCTTTTCTGGGAAGAGCCCCGTCACCACACTGCTTGAGTGTATGC
ACAAATTGGGGAACTCCTGCGAATTCCGTCTCCTGTCCAAAGAAGGCCCT
GCCCATGAACCCAAGTTCCAATACTGTGTTGCAGTGGGAGCCCAAACTTT
CCCCAGTGTGAGTGCTCCCAGCAAGAAAGTGGCAAAGCAGATGGCCGCAG
AGGAAGCCATGAAGGCCCTGCATGGGGAGGCGACCAACTCCATGGCTTCT
GATAACCAGCCTGAAGGTATGATCTCAGAGTCACTTGATAACTTGGAATC
CATGATGCCCAACAAGGTCAGGAAGATTGGCGAGCTCGTGAGATACCTGA
ACACCAACCCTGTGGGTGGCCTTTTGGAGTACGCCCGCTCCCATGGCTTT
GCTGCTGAATTCAAGTTGGTCGACCAGTCCGGACCTCCTCACGAGCCCAA
GTTCGTTTACCAAGCAAAAGTTGGGGGTCGCTGGTTCCCAGCCGTCTGCG
CACACAGCAAGAAGCAAGGCAAGCAGGAAGCAGCAGATGCGGCTCTCCGT
GTCTTGATTGGGGAGAACGAGAAGGCAGAACGCATGGGTTTCACAGAGGT
AACCCCAGTGACAGGGGCCAGTCTCAGAAGAACTATGCTCCTCCTCTCAA
GGTCCCCAGAAGCACAGCCAAAGACACTCCCTCTCACTGGCAGCACCTTC
CATGACCAGATAGCCATGCTGAGCCACCGGTGCTTCAACACTCTGACTAA
CAGCTTCCAGCCCTCCTTGCTCGGCCGCAAGATTCTGGCCGCCATCATTA
TGAAAAAAGACTCTGAGGACATGGGTGTCGTCGTCAGCTTGGGAACAGGG
AATCGCTGTGTGAAAGGAGATTCTCTCAGCCTAAAAGGAGAAACTGTCAA
TGACTGCCATGCAGAAATAATCTCCCGGAGAGGCTTCATCAGGTTTCTCT
ACAGTGAGTTAATGAAATACAACTCCCAGACTGCGAAGGATAGTATATTT
GAACCTGCTAAGGGAGGAGAAAAGCTCCAAATAAAAAAGACTGTGTCATT
CCATCTGTATATCAGCACTGCTCCGTGTGGAGATGGCGCCCTCTTTGACA
AGTCCTGCAGCGACCGTGCTATGGAAAGCACAGAATCCCGCCACTACCCT
GTCTTCGAGAATCCCAAACAAGGAAAGCTCCGCACCAAGGTGGAGAACGG
AGAAGGCACAATCCCTGTGGAATCCAGTGACATTGTGCCTACGTGGGATG
GCATTCGGCTCGGGGAGAGACTCCGTACCATGTCCTGTAGTGACAAAATC
CTACGCTGGAACGTGCTGGGCCTGCAAGGGGCACTGTTGACCCACTTCCT
GCAGCCCATTTATCTCAAATCTGTCACATTGGGTTACCTTTTCAGCCAAG
GGCATCTGACCCGTGCTATTTGCTGTCGTGTGACAAGAGATGGGAGTGCA
TTTGAGGATGGACTACGACATCCCTTTATTGTCAACCACCCCAAGGTTGG
CAGAGTCAGCATATATGATTCCAAAAGGCAATCCGGGAAGACTAAGGAGA
CAAGCGTCAACTGGTGTCTGGCTGATGGCTATGACCTGGAGATCCTGGAC
GGTACCAGAGGCACTGTGGATGGGCCACGGAATGAATTGTCCCGGGTCTC
CAAAAAGAACATTTTTCTTCTATTTAAGAAGCTCTGCTCCTTCCGTTACC
GCAGGGATCTACTGAGACTCTCCTATGGTGAGGCCAAGAAAGCTGCCCGT
GACTACGAGACGGCCAAGAACTACTTCAAAAAAGGCCTGAAGGATATGGG
CTATGGGAACTGGATTAGCAAACCCCAGGAGGAAAAGAACTTTTATCTCT
GCCCAGTATAGTATGCTCCAGTGACAGATGGATTAGGGTGTGTCATACTA
GGGTGTGAGAGAGGTAGGTCGTAGCATTCCTCATCACATGGTCAGGGGAT
TTTTTTTTCTCCTTTTTTTTTCTTTTTAAGCCATAATTGGTGATACTGAA
AACTTTGGGTTCCCATTTATCCTGCTTTCTTTGGGATTGCTAGGCAAGGT
CTGGCCAGGCCCCCCTTTTTTCCCCCAAGTGAAGAGGCAGAAACCTAAGA
AGTTATCTTTTCTTTCTACCCAAAGCATACATAGTCACTGAGCACCTGCG
GTCCATTTCCTCTTAAAAGTTTTGTTTTGATTTGTTTCCATTTCCTTTCC
CTTTGTGTTTGCTACACTGACCTCTTGCGGTCTTGATTAGGTTTCAGTCA
ACTCTGGATCATGTCAGGGACTGATAATTTCATTTGTGGATTACGCAGAC
CCCTCTACTTCCCCTCTTTCCCTTCTGAGATTCTTTCCTTGTGATCTGAA
TGTCTCCTTTTCCCCCTCAGAGGGCAAAGAGGTGAACATAAAGGATTTGG
TGAAACATTTGTAAGGGTAGGAGTTGAAAACTGCAGTTCCCAGTGCCACG
GAAGTGTGATTGGAGCCTGCAGATAATGCCCAGCCATCCTCCCATCCTGC
ACTTTAGCCAGCTGCAGGGCGGGCAAGGCAAGGAAAGCTGCTTCCCTGGA
AGTGTATCACTTTCTCCGGCAGCTGGGAAGTCTAGAACCAGCCAGACTGG
GTTAAGGGAGCTGCTCAAGCAATAGCAGAGGTTTCACCCGGCAGGATGAC
ACAGACCACTTCCCAGGGAGCACGGGCATGCCTTGGAATATTGCCAAGCT
TCCAGCTGCCTCTTCTCCTAAAGCATTCCTAGGAATATTTTCCCCGCCAA
TGCTGGGCGTACACCCTAGCCAACGGGACAAATCCTAGAGGGTATAAAAT
CATCTCTGCTCAGATAATCATGACTTAGCAAGAATAAGGGCAAAAAATCC
TGTTGGCTTAACGTCACTGTTCCACCCGGTGTAATATCTCTCATGACAGT
GACACCAAGGGAAGTTGACTAAGTCACATGTAAATTAGGAGTGTTTTAAA
GAATGCCATAGATGTTGATTCTTAACTGCTACAGATAACCTGTAATTGAG
CAGATTTAAAATTCAGGCATACTTTTCCATTTATCCAAGTGCTTTCATTT
TTCCAGATGGCTTCAGAAGTAGGCTCGTGGGCAGGGCGCAGACCTGATCT
TTATAGGGTTGACATAGAAAGCAGTAGTTGTGGGTGAAAGGGCAGGTTGT
CTTCAAACTCTGTGAGGTAGAATCCTTTGTCTATACCTCCATGAACATTG
ACTCGTGTGTTCAGAGCCTTTGGCCTCTCTGTGGAGTCTGGCTCTCTGGC
TCCTGTGCATTCTTTGAATAGTCACTCGTAAAAACTGTCAGTGCTTGAAA
CTGTTTCCTTTACTCATGTTGAAGGGACTTTGTTGGCTTTTAGAGTGTTG
GTCATGACTCCAAGAGCAGAGCAGGGAAGAGCCCAAGCATAGACTTGGTG
CCGTGGTGATGGCTGCAGTCCAGTTTTGTGATGCTGCTTTTACGTGTCCC
TCGATAACAGTCAGCTAGACACACTCAGGAGGACTACTGAGGCTCTGCGA
CCTTCAGGAGCTGAGCCTGCCTCTCTCCTTTAGATGACAGACCTTCATCT
GGGAACGTGCTGAGCCAGCACCCTCAGATGATTTCCCTCCAAACTGCTGA
CTAGGTCATCCTCTGTCTGGTAGAGACATTCACATCTTTGCTTTTATTCT
ATGCTCTCTGTACTTTTGACCAAAAATTGACCAAAGTAAGAAAATGCAAG
TTCTAAAAATAGACTAAGGATGCCTTTGCAGAACACCAAAGCATCCCAAG
GAACTGGTAGGGAAGTGGCGCCTGTCTCCTGGAGTGGAAGAGGCCTGCTC
CCTGGCTCTGGGTCTGCTGGGGGCACAGTAAATCAGTCTTGGCACCCACA
TCCAGGGCAGAGAGGTCTGTGGTTCTCAGCATCAGAAGGCAGCGCAGCCC
CTCTCCTCTTCAGGCTACAGGGTTGTCACCTGCTGAGTCCTCAGGTTGTT
TGGCCTCTCTGGTCCATCTTGGGCATTAGGTTCTCCAGCAGAGCTCTGGC
CAGCTGCCTCTTCTTTAACTGGGAACACAGGCTCTCACAAGATCAGAACC
CCCACTCACCCCCAAGATCTTATCTAGCAAGCCTGTAGTATTCAGTTTCT
GTTGTAGGAAGAGAGCGAGGCATCCCTGAATTCCACGCATCTGCTGGAAA
CGAGCCGTGTCAGATCGCACATCCCTGCGCCCCCATGCCCCTCTGAGTCA
CACAGGACAGAGGAGGCAGAGCTTCTGCCCACTGTTATCTTCACTTTCTT
TGTCCAGTCTTTTGTTTTTAATAAGCAGTGACCCTCCCTACTCTTCTTTT
TAATGATTTTTGTAGTTGATTTGTCTGAACTGTGGCTACTGTGCATTCCT
TGAATAATCACTTGTAAAAATTGTCAGTGCTTGAAGCTGTTTCCTTTACT
CACATTGAAGGGACTTCGTTGGTTTTTTGGAGTCTTGGTTGTGACTCCAA
GAGCAGAGTGAGGAAGACCCCCAAGCATAGACTCGGGTACTGTGATGATG
GCTGCAGTCCAGTTTTATGATTCTGCTTTTATGTGTCCCTTGATAACAGT
GACTTAACAATATACATTCCTCATAAATAAAAAAAAAACAAGAATCTGAA
TTCTTAGAAA

SEQ ID NO: 3 - Human ADAR1 - Exemplary Transcript Variant 1 Coding Sequence

ATGAATCCGCGGCAGGGGTATTCCCTCAGCGGATACTACACCCATCCATT
TCAAGGCTATGAGCACAGACAGCTCAGATACCAGCAGCCTGGGCCAGGAT
CTTCCCCCAGTAGTTTCCTGCTTAAGCAAATAGAATTTCTCAAGGGGCAG
CTCCCAGAAGCACCGGTGATTGGAAAGCAGACACCGTCACTGCCACCTTC
CCTCCCAGGACTCCGGCCAAGGTTTCCAGTACTACTTGCCTCCAGTACCA
GAGGCAGGCAAGTGGACATCAGGGGTGTCCCCAGGGGCGTGCATCTCGGA
AGTCAGGGGCTCCAGAGAGGGTTCCAGCATCCTTCACCACGTGGCAGGAG
TCTGCCACAGAGAGGTGTTGATTGCCTTTCCTCACATTTCCAGGAACTGA
GTATCTACCAAGATCAGGAACAAAGGATCTTAAAGTTCCTGGAAGAGCTT
GGGGAAGGGAAGGCCACCACAGCACATGATCTGTCTGGGAAACTTGGGAC
TCCGAAGAAAGAAATCAATCGAGTTTTATACTCCCTGGCAAAGAAGGGCA
AGCTACAGAAAGAGGCAGGAACACCCCCTTTGTGGAAAATCGCGGTCTCC
ACTCAGGCTTGGAACCAGCACAGCGGAGTGGTAAGACCAGACGGTCATAG
CCAAGGAGCCCCAAACTCAGACCCGAGTTTGGAACCGGAAGACAGAAACT
CCACATCTGTCTCAGAAGATCTTCTTGAGCCTTTTATTGCAGTCTCAGCT
CAGGCTTGGAACCAGCACAGCGGAGTGGTAAGACCAGACAGTCATAGCCA
AGGATCCCCAAACTCAGACCCAGGTTTGGAACCTGAAGACAGCAACTCCA
CATCTGCCTTGGAAGATCCTCTTGAGTTTTTAGACATGGCCGAGATCAAG
GAGAAAATCTGCGACTATCTCTTCAATGTGTCTGACTCCTCTGCCCTGAA
TTTGGCTAAAAATATTGGCCTTACCAAGGCCCGAGATATAAATGCTGTGC
TAATTGACATGGAAAGGCAGGGGGATGTCTATAGACAAGGGACAACCCCT
CCCATATGGCATTTGACAGACAAGAAGCGAGAGAGGATGCAAATCAAGAG
AAATACGAACAGTGTTCCTGAAACCGCTCCAGCTGCAATCCCTGAGACCA
AAAGAAACGCAGAGTTCCTCACCTGTAATATACCCACATCAAATGCCTCA
AATAACATGGTAACCACAGAAAAAGTGGAGAATGGGCAGGAACCTGTCAT
AAAGTTAGAAAACAGGCAAGAGGCCAGACCAGAACCAGCAAGACTGAAAC
CACCTGTTCATTACAATGGCCCCTCAAAAGCAGGGTATGTTGACTTTGAA
AATGGCCAGTGGGCCACAGATGACATCCCAGATGACTTGAATAGTATCCG
CGCAGCACCAGGTGAGTTTCGAGCCATCATGGAGATGCCCTCCTTCTACA
GTCATGGCTTGCCACGGTGTTCACCCTACAAGAAACTGACAGAGTGCCAG
CTGAAGAACCCCATCAGCGGGCTGTTAGAATATGCCCAGTTCGCTAGTCA
AACCTGTGAGTTCAACATGATAGAGCAGAGTGGACCACCCCATGAACCTC
GATTTAAATTCCAGGTTGTCATCAATGGCCGAGAGTTTCCCCCAGCTGAA
GCTGGAAGCAAGAAAGTGGCCAAGCAGGATGCAGCTATGAAAGCCATGAC
AATTCTGCTAGAGGAAGCCAAAGCCAAGGACAGTGGAAAATCAGAAGAAT
CATCCCACTATTCCACAGAGAAAGAATCAGAGAAGACTGCAGAGTCCCAG
ACCCCCACCCCTTCAGCCACATCCTTCTTTTCTGGGAAGAGCCCCGTCAC
CACACTGCTTGAGTGTATGCACAAATTGGGGAACTCCTGCGAATTCCGTC
TCCTGTCCAAAGAAGGCCCTGCCCATGAACCCAAGTTCCAATACTGTGTT
GCAGTGGGAGCCCAAACTTTCCCCAGTGTGAGTGCTCCCAGCAAGAAAGT
GGCAAAGCAGATGGCCGCAGAGGAAGCCATGAAGGCCCTGCATGGGGAGG
CGACCAACTCCATGGCTTCTGATAACCAGCCTGAAGGTATGATCTCAGAG
TCACTTGATAACTTGGAATCCATGATGCCCAACAAGGTCAGGAAGATTGG
CGAGCTCGTGAGATACCTGAACACCAACCCTGTGGGTGGCCTTTTGGAGT
ACGCCCGCTCCCATGGCTTTGCTGCTGAATTCAAGTTGGTCGACCAGTCC
GGACCTCCTCACGAGCCCAAGTTCGTTTACCAAGCAAAAGTTGGGGGTCG
CTGGTTCCCAGCCGTCTGCGCACACAGCAAGAAGCAAGGCAAGCAGGAAG
CAGCAGATGCGGCTCTCCGTGTCTTGATTGGGGAGAACGAGAAGGCAGAA
CGCATGGGTTTCACAGAGGTAACCCCAGTGACAGGGGCCAGTCTCAGAAG
AACTATGCTCCTCCTCTCAAGGTCCCCAGAAGCACAGCCAAAGACACTCC
CTCTCACTGGCAGCACCTTCCATGACCAGATAGCCATGCTGAGCCACCGG
TGCTTCAACACTCTGACTAACAGCTTCCAGCCCTCCTTGCTCGGCCGCAA
GATTCTGGCCGCCATCATTATGAAAAAAGACTCTGAGGACATGGGTGTCG
TCGTCAGCTTGGGAACAGGGAATCGCTGTGTGAAAGGAGATTCTCTCAGC
CTAAAAGGAGAAACTGTCAATGACTGCCATGCAGAAATAATCTCCCGGAG
AGGCTTCATCAGGTTTCTCTACAGTGAGTTAATGAAATACAACTCCCAGA
CTGCGAAGGATAGTATATTTGAACCTGCTAAGGGAGGAGAAAAGCTCCAA
ATAAAAAAGACTGTGTCATTCCATCTGTATATCAGCACTGCTCCGTGTGG
AGATGGCGCCCTCTTTGACAAGTCCTGCAGCGACCGTGCTATGGAAAGCA
CAGAATCCCGCCACTACCCTGTCTTCGAGAATCCCAAACAAGGAAAGCTC
CGCACCAAGGTGGAGAACGGAGAAGGCACAATCCCTGTGGAATCCAGTGA
CATTGTGCCTACGTGGGATGGCATTCGGCTCGGGGAGAGACTCCGTACCA
TGTCCTGTAGTGACAAAATCCTACGCTGGAACGTGCTGGGCCTGCAAGGG
GCACTGTTGACCCACTTCCTGCAGCCCATTTATCTCAAATCTGTCACATT
GGGTTACCTTTTCAGCCAAGGGCATCTGACCCGTGCTATTTGCTGTCGTG
TGACAAGAGATGGGAGTGCATTTGAGGATGGACTACGACATCCCTTTATT
GTCAACCACCCCAAGGTTGGCAGAGTCAGCATATATGATTCCAAAAGGCA
ATCCGGGAAGACTAAGGAGACAAGCGTCAACTGGTGTCTGGCTGATGGCT
ATGACCTGGAGATCCTGGACGGTACCAGAGGCACTGTGGATGGGCCACGG
AATGAATTGTCCCGGGTCTCCAAAAAGAACATTTTTCTTCTATTTAAGAA
GCTCTGCTCCTTCCGTTACCGCAGGGATCTACTGAGACTCTCCTATGGTG
AGGCCAAGAAAGCTGCCCGTGACTACGAGACGGCCAAGAACTACTTCAAA
AAAGGCCTGAAGGATATGGGCTATGGGAACTGGATTAGCAAACCCCAGGA
GGAAAAGAACTTTTATCTCTGCCCAGTATAG

SEQ ID NO: 4 - Human ADAR1 - Transcript Variant 1 Coding Sequence

ATGAATCCGCGGCAGGGGTATTCCCTCAGCGGATACTACACCCATCCATT
TCAAGGCTATGAGCACAGACAGCTCAGGTACCAGCAGCCTGGGCCAGGAT
CTTCCCCCAGTAGTTTCCTGCTTAAGCAAATAGAATTTCTCAAGGGGCAG
CTCCCAGAAGCACCGGTGATTGGAAAGCAGACACCGTCACTGCCACCTTC
CCTCCCAGGACTCCGGCCAAGGTTTCCAGTACTACTTGCCTCCAGTACCA
GAGGCAGGCAAGTGGACATCAGGGGTGTCCCCAGGGGCGTGCATCTCAGA
AGTCAGGGGCTCCAGAGAGGGTTCCAGCATCCTTCACCACGTGGCAGGAG
TCTGCCACAGAGAGGTGTTGATTGCCTTTCCTCACATTTCCAGGAACTGA
GTATCTACCAAGATCAGGAACAAAGGATCTTAAAGTTCCTGGAAGAGCTT
GGGGAAGGGAAGGCCACCACAGCACATGATCTGTCTGGGAAACTTGGGAC
TCCGAAGAAAGAAATCAATCGAGTTTTATACTCCCTGGCAAAGAAGGGCA
AGCTACAGAAAGAGGCAGGAACACCCCCTTTGTGGAAAATCGCGGTCTCC
ACTCAGGCTTGGAACCAGCACAGCGGAGTGGTAAGACCAGACGGTCATAG
CCAAGGAGCCCCAAACTCAGACCCGAGTTTGGAACCGGAAGACAGAAACT
CCACATCTGTCTCAGAAGATCTTCTTGAGCCTTTTATTGCAGTCTCAGCT
CAGGCTTGGAACCAGCACAGCGGAGTGGTAAGACCAGACAGTCATAGCCA
AGGATCCCCAAACTCAGACCCAGGTTTGGAACCTGAAGACAGCAACTCCA
CATCTGCCTTGGAAGATCCTCTTGAGTTTTTAGACATGGCCGAGATCAAG
GAGAAAATCTGCGACTATCTCTTCAATGTGTCTGACTCCTCTGCCCTGAA
TTTGGCTAAAAATATTGGCCTTACCAAGGCCCGAGATATAAATGCTGTGC
TAATTGACATGGAAAGGCAGGGGGATGTCTATAGACAAGGGACAACCCCT
CCCATATGGCATTTGACAGACAAGAAGCGAGAGAGGATGCAAATCAAGAG
AAATACGAACAGTGTTCCTGAAACCGCTCCAGCTGCAATCCCTGAGACCA
AAAGAAACGCAGAGTTCCTCACCTGTAATATACCCACATCAAATGCCTCA
AATAACATGGTAACCACAGAAAAAGTGGAGAATGGGCAGGAACCTGTCAT
AAAGTTAGAAAACAGGCAAGAGGCCAGACCAGAACCAGCAAGACTGAAAC
CACCTGTTCATTACAATGGCCCCTCAAAAGCAGGGTATGTTGACTTTGAA
AATGGCCAGTGGGCCACAGATGACATCCCAGATGACTTGAATAGTATCCG
CGCAGCACCAGGTGAGTTTCGAGCCATCATGGAGATGCCCTCCTTCTACA
GTCATGGCTTGCCACGGTGTTCACCCTACAAGAAACTGACAGAGTGCCAG
CTGAAGAACCCCATCAGCGGGCTGTTAGAATATGCCCAGTTCGCTAGTCA
AACCTGTGAGTTCAACATGATAGAGCAGAGTGGACCACCCCATGAACCTC
GATTTAAATTCCAGGTTGTCATCAATGGCCGAGAGTTTCCCCCAGCTGAA
GCTGGAAGCAAGAAAGTGGCCAAGCAGGATGCAGCTATGAAAGCCATGAC
AATTCTGCTAGAGGAAGCCAAAGCCAAGGACAGTGGAAAATCAGAAGAAT
CATCCCACTATTCCACAGAGAAAGAATCAGAGAAGACTGCAGAGTCCCAG
ACCCCCACCCCTTCAGCCACATCCTTCTTTTCTGGGAAGAGCCCCGTCAC
CACACTGCTTGAGTGTATGCACAAATTGGGGAACTCCTGCGAATTCCGTC
TCCTGTCCAAAGAAGGCCCTGCCCATGAACCCAAGTTCCAATACTGTGTT
GCAGTGGGAGCCCAAACTTTCCCCAGTGTGAGTGCTCCCAGCAAGAAAGT
GGCAAAGCAGATGGCCGCAGAGGAAGCCATGAAGGCCCTGCATGGGGAGG
CGACCAACTCCATGGCTTCTGATAACCAGCCTGAAGGTATGATCTCAGAG
TCACTTGATAACTTGGAATCCATGATGCCCAACAAGGTCAGGAAGATTGG
CGAGCTCGTGAGATACCTGAACACCAACCCTGTGGGTGGCCTTTTGGAGT
ACGCCCGCTCCCATGGCTTTGCTGCTGAATTCAAGTTGGTCGACCAGTCC
GGACCTCCTCACGAGCCCAAGTTCGTTTACCAAGCAAAAGTTGGGGGTCG
CTGGTTCCCAGCCGTCTGCGCACACAGCAAGAAGCAAGGCAAGCAGGAAG
CAGCAGATGCGGCTCTCCGTGTCTTGATTGGGGAGAACGAGAAGGCAGAA
CGCATGGGTTTCACAGAGGTAACCCCAGTGACAGGGGCCAGTCTCAGAAG
AACTATGCTCCTCCTCTCAAGGTCCCCAGAAGCACAGCCAAAGACACTCC
CTCTCACTGGCAGCACCTTCCATGACCAGATAGCCATGCTGAGCCACCGG
TGCTTCAACACTCTGACTAACAGCTTCCAGCCCTCCTTGCTCGGCCGCAA
GATTCTGGCCGCCATCATTATGAAAAAAGACTCTGAGGACATGGGTGTCG
TCGTCAGCTTGGGAACAGGGAATCGCTGTGTGAAAGGAGATTCTCTCAGC
CTAAAAGGAGAAACTGTCAATGACTGCCATGCAGAAATAATCTCCCGGAG
AGGCTTCATCAGGTTTCTCTACAGTGAGTTAATGAAATACAACTCCCAGA
CTGCGAAGGATAGTATATTTGAACCTGCTAAGGGAGGAGAAAAGCTCCAA
ATAAAAAAGACTGTGTCATTCCATCTGTATATCAGCACTGCTCCGTGTGG
AGATGGCGCCCTCTTTGACAAGTCCTGCAGCGACCGTGCTATGGAAAGCA
CAGAATCCCGCCACTACCCTGTCTTCGAGAATCCCAAACAAGGAAAGCTC
CGCACCAAGGTGGAGAACGGAGAAGGCACAATCCCTGTGGAATCCAGTGA
CATTGTGCCTACGTGGGATGGCATTCGGCTCGGGGAGAGACTCCGTACCA
TGTCCTGTAGTGACAAAATCCTACGCTGGAACGTGCTGGGCCTGCAAGGG
GCACTGTTGACCCACTTCCTGCAGCCCATTTATCTCAAATCTGTCACATT
GGGTTACCTTTTCAGCCAAGGGCATCTGACCCGTGCTATTTGCTGTCGTG
TGACAAGAGATGGGAGTGCATTTGAGGATGGACTACGACATCCCTTTATT
GTCAACCACCCCAAGGTTGGCAGAGTCAGCATATATGATTCCAAAAGGCA
ATCCGGGAAGACTAAGGAGACAAGCGTCAACTGGTGTCTGGCTGATGGCT
ATGACCTGGAGATCCTGGACGGTACCAGAGGCACTGTGGATGGGCCACGG
AATGAATTGTCCCGGGTCTCCAAAAAGAACATTTTTCTTCTATTTAAGAA
GCTCTGCTCCTTCCGTTACCGCAGGGATCTACTGAGACTCTCCTATGGTG
AGGCCAAGAAAGCTGCCCGTGACTACGAGACGGCCAAGAACTACTTCAAA
AAAGGCCTGAAGGATATGGGCTATGGGAACTGGATTAGCAAACCCCAGGA
GGAAAAGAACTTTTATCTCTGCCCAGTATAG

SEQ ID NO: 5 - Human ADAR1 - Exemplary Transcript Variant 1 Amino Acid Sequence (aka isoform-a, and/or p150)

MNPRQGYSLSGYYTHPFQGYEHRQLRYQQPGPGSSPSSFLLKQIEFLKGQ
LPEAPVIGKQTPSLPPSLPGLRPRFPVLLASSTRGRQVDIRGVPRGVHLG
SQGLQRGFQHPSPRGRSLPQRGVDCLSSHFQELSIYQDQEQRILKFLEEL
GEGKATTAHDLSGKLGTPKKEINRVLYSLAKKGKLQKEAGTPPLWKIAVS
TQAWNQHSGVVRPDGHSQGAPNSDPSLEPEDRNSTSVSEDLLEPFIAVSA
QAWNQHSGVVRPDSHSQGSPNSDPGLEPEDSNSTSALEDPLEFLDMAEIK
EKICDYLFNVSDSSALNLAKNIGLTKARDINAVLIDMERQGDVYRQGTTP
PIWHLTDKKRERMQIKRNTNSVPETAPAAIPETKRNAEFLTCNIPTSNAS
NNMVTTEKVENGQEPVIKLENRQEARPEPARLKPPVHYNGPSKAGYVDFE
NGQWATDDIPDDLNSIRAAPGEFRAIMEMPSFYSHGLPRCSPYKKLTECQ
LKNPISGLLEYAQFASQTCEFNMIEQSGPPHEPRFKFQVVINGREFPPAE
AGSKKVAKQDAAMKAMTILLEEAKAKDSGKSEESSHYSTEKESEKTAESQ
TPTPSATSFFSGKSPVTTLLECMHKLGNSCEFRLLSKEGPAHEPKFQYCV
AVGAQTFPSVSAPSKKVAKQMAAEEAMKALHGEATNSMASDNQPEGMISE
SLDNLESMMPNKVRKIGELVRYLNTNPVGGLLEYARSHGFAAEFKLVDQS
GPPHEPKFVYQAKVGGRWFPAVCAHSKKQGKQEAADAALRVLIGENEKAE
RMGFTEVTPVTGASLRRTMLLLSRSPEAQPKTLPLTGSTFHDQIAMLSHR
CFNTLTNSFQPSLLGRKILAAIIMKKDSEDMGVVVSLGTGNRCVKGDSLS
LKGETVNDCHAEIISRRGFIRFLYSELMKYNSQTAKDSIFEPAKGGEKLQ
IKKTVSFHLYISTAPCGDGALFDKSCSDRAMESTESRHYPVFENPKQGKL
RTKVENGEGTIPVESSDIVPTWDGIRLGERLRTMSCSDKILRWNVLGLQG
ALLTHFLQPIYLKSVTLGYLFSQGHLTRAICCRVTRDGSAFEDGLRHPFI
VNHPKVGRVSIYDSKRQSGKTKETSVNWCLADGYDLEILDGTRGTVDGPR
NELSRVSKKNIFLLFKKLCSFRYRRDLLRLSYGEAKKAARDYETAKNYFK
KGLKDMGYGNWISKPQEEKNFYLCPV

SEQ ID NO: 6 - Human ADAR1 - Transcript Variant 1 Amino Acid Sequence (aka isoform-a, and/or p150)

MNPRQGYSLSGYYTHPFQGYEHRQLRYQQPGPGSSPSSFLLKQIEFLKGQ
LPEAPVIGKQTPSLPPSLPGLRPRFPVLLASSTRGRQVDIRGVPRGVHLR
SQGLQRGFQHPSPRGRSLPQRGVDCLSSHFQELSIYQDQEQRILKFLEEL
GEGKATTAHDLSGKLGTPKKEINRVLYSLAKKGKLQKEAGTPPLWKIAVS
TQAWNQHSGVVRPDGHSQGAPNSDPSLEPEDRNSTSVSEDLLEPFIAVSA
QAWNQHSGVVRPDSHSQGSPNSDPGLEPEDSNSTSALEDPLEFLDMAEIK
EKICDYLFNVSDSSALNLAKNIGLTKARDINAVLIDMERQGDVYRQGTTP
PIWHLTDKKRERMQIKRNTNSVPETAPAAIPETKRNAEFLTCNIPTSNAS
NNMVTTEKVENGQEPVIKLENRQEARPEPARLKPPVHYNGPSKAGYVDFE
NGQWATDDIPDDLNSIRAAPGEFRAIMEMPSFYSHGLPRCSPYKKLTECQ
LKNPISGLLEYAQFASQTCEFNMIEQSGPPHEPRFKFQVVINGREFPPAE
AGSKKVAKQDAAMKAMTILLEEAKAKDSGKSEESSHYSTEKESEKTAESQ
TPTPSATSFFSGKSPVTTLLECMHKLGNSCEFRLLSKEGPAHEPKFQYCV
AVGAQTFPSVSAPSKKVAKQMAAEEAMKALHGEATNSMASDNQPEGMISE
SLDNLESMMPNKVRKIGELVRYLNTNPVGGLLEYARSHGFAAEFKLVDQS
GPPHEPKFVYQAKVGGRWFPAVCAHSKKQGKQEAADAALRVLIGENEKAE
RMGFTEVTPVTGASLRRTMLLLSRSPEAQPKTLPLTGSTFHDQIAMLSHR
CFNTLTNSFQPSLLGRKILAAIIMKKDSEDMGVVVSLGTGNRCVKGDSLS
LKGETVNDCHAEIISRRGFIRFLYSELMKYNSQTAKDSIFEPAKGGEKLQ
IKKTVSFHLYISTAPCGDGALFDKSCSDRAMESTESRHYPVFENPKQGKL
RTKVENGEGTIPVESSDIVPTWDGIRLGERLRTMSCSDKILRWNVLGLQG
ALLTHFLQPIYLKSVTLGYLFSQGHLTRAICCRVTRDGSAFEDGLRHPFI
VNHPKVGRVSIYDSKRQSGKTKETSVNWCLADGYDLEILDGTRGTVDGPR
NELSRVSKKNIFLLFKKLCSFRYRRDLLRLSYGEAKKAARDYETAKNYFK
KGLKDMGYGNWISKPQEEKNFYLCPV

In certain embodiments, an ADAR1 polynucleotide or a human ADAR1 gene incorporated into a non-human animal (e.g., a rodent, e.g., a rat or mouse) is represented by or comprises a sequence encoding a human ADAR1 transcript variant 2 or a characteristic portion thereof. In certain embodiments, ADAR1 polynucleotide or a human ADAR1 gene comprises, or consists of, a nucleotide sequence that is the same as, differs by no more than 1, 2, 3, 4, 5, 6, 7, 8, 9, or 10 nucleotides from, or has at least has at least 90%, 92%, 94%, 96%, 98%, or 99% homology with, the nucleotide sequence of SEQ ID NO: 7 or SEQ ID NO: 8 or a characteristic portion thereof. In some embodiments, a ADAR1 polynucleotide or a human ADAR1 gene comprises, or consists of, a nucleotide sequence that is the same as the nucleotide sequence of S SEQ ID NO: 7 or SEQ ID NO: 8 or a characteristic portion thereof. In certain embodiments, an encoded human ADAR1 Amino Acid sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the Amino Acid sequence of SEQ ID NO: 9 or a characteristic portion thereof. In certain embodiments, the amino acid sequence of an encoded and/or expressed ADAR1 polypeptide or a characteristic portion thereof, is or comprises a sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the amino acid sequence of SEQ ID NO: 9 or a characteristic portion thereof.

SEQ ID NO: 7 - Human ADAR1 - Transcript Variant 2 cDNA Sequence

GAACCGGAGCCATCTTGGGCCCGGCGCGCAGACCCGCGGAGTTTCCCGTG
CCGACGCCCCGGGGCCACTTCCAGTGCGGAGTAGCGGAGGCGTGGGGGCC
TCGAGGGGCTGGCGCGGCCCAGCGGTCGGGCCAGGGTCGTGCCGCCGGCG
GGTCGGGCCGGGCAATGCCTCGCGGGCGCAATGAATCCGCGGCAGGGGTA
TTCCCTCAGCGGATACTACACCCATCCATTTCAAGGCTATGAGCACAGAC
AGCTCAGGTACCAGCAGCCTGGGCCAGGATCTTCCCCCAGTAGTTTCCTG
CTTAAGCAAATAGAATTTCTCAAGGGGCAGCTCCCAGAAGCACCGGTGAT
TGGAAAGCAGACACCGTCACTGCCACCTTCCCTCCCAGGACTCCGGCCAA
GGTTTCCAGTACTACTTGCCTCCAGTACCAGAGGCAGGCAAGTGGACATC
AGGGGTGTCCCCAGGGGCGTGCATCTCAGAAGTCAGGGGCTCCAGAGAGG
GTTCCAGCATCCTTCACCACGTGGCAGGAGTCTGCCACAGAGAGGTGTTG
ATTGCCTTTCCTCACATTTCCAGGAACTGAGTATCTACCAAGATCAGGAA
CAAAGGATCTTAAAGTTCCTGGAAGAGCTTGGGGAAGGGAAGGCCACCAC
AGCACATGATCTGTCTGGGAAACTTGGGACTCCGAAGAAAGAAATCAATC
GAGTTTTATACTCCCTGGCAAAGAAGGGCAAGCTACAGAAAGAGGCAGGA
ACACCCCCTTTGTGGAAAATCGCGGTCTCCACTCAGGCTTGGAACCAGCA
CAGCGGAGTGGTAAGACCAGACGGTCATAGCCAAGGAGCCCCAAACTCAG
ACCCGAGTTTGGAACCGGAAGACAGAAACTCCACATCTGTCTCAGAAGAT
CTTCTTGAGCCTTTTATTGCAGTCTCAGCTCAGGCTTGGAACCAGCACAG
CGGAGTGGTAAGACCAGACAGTCATAGCCAAGGATCCCCAAACTCAGACC
CAGGTTTGGAACCTGAAGACAGCAACTCCACATCTGCCTTGGAAGATCCT
CTTGAGTTTTTAGACATGGCCGAGATCAAGGAGAAAATCTGCGACTATCT
CTTCAATGTGTCTGACTCCTCTGCCCTGAATTTGGCTAAAAATATTGGCC
TTACCAAGGCCCGAGATATAAATGCTGTGCTAATTGACATGGAAAGGCAG
GGGGATGTCTATAGACAAGGGACAACCCCTCCCATATGGCATTTGACAGA
CAAGAAGCGAGAGAGGATGCAAATCAAGAGAAATACGAACAGTGTTCCTG
AAACCGCTCCAGCTGCAATCCCTGAGACCAAAAGAAACGCAGAGTTCCTC
ACCTGTAATATACCCACATCAAATGCCTCAAATAACATGGTAACCACAGA
AAAAGTGGAGAATGGGCAGGAACCTGTCATAAAGTTAGAAAACAGGCAAG
AGGCCAGACCAGAACCAGCAAGACTGAAACCACCTGTTCATTACAATGGC
CCCTCAAAAGCAGGGTATGTTGACTTTGAAAATGGCCAGTGGGCCACAGA
TGACATCCCAGATGACTTGAATAGTATCCGCGCAGCACCAGGTGAGTTTC
GAGCCATCATGGAGATGCCCTCCTTCTACAGTCATGGCTTGCCACGGTGT
TCACCCTACAAGAAACTGACAGAGTGCCAGCTGAAGAACCCCATCAGCGG
GCTGTTAGAATATGCCCAGTTCGCTAGTCAAACCTGTGAGTTCAACATGA
TAGAGCAGAGTGGACCACCCCATGAACCTCGATTTAAATTCCAGGTTGTC
ATCAATGGCCGAGAGTTTCCCCCAGCTGAAGCTGGAAGCAAGAAAGTGGC
CAAGCAGGATGCAGCTATGAAAGCCATGACAATTCTGCTAGAGGAAGCCA
AAGCCAAGGACAGTGGAAAATCAGAAGAATCATCCCACTATTCCACAGAG
AAAGAATCAGAGAAGACTGCAGAGTCCCAGACCCCCACCCCTTCAGCCAC
ATCCTTCTTTTCTGGGAAGAGCCCCGTCACCACACTGCTTGAGTGTATGC
ACAAATTGGGGAACTCCTGCGAATTCCGTCTCCTGTCCAAAGAAGGCCCT
GCCCATGAACCCAAGTTCCAATACTGTGTTGCAGTGGGAGCCCAAACTTT
CCCCAGTGTGAGTGCTCCCAGCAAGAAAGTGGCAAAGCAGATGGCCGCAG
AGGAAGCCATGAAGGCCCTGCATGGGGAGGCGACCAACTCCATGGCTTCT
GATAACCAGCCTGAAGGTATGATCTCAGAGTCACTTGATAACTTGGAATC
CATGATGCCCAACAAGGTCAGGAAGATTGGCGAGCTCGTGAGATACCTGA
ACACCAACCCTGTGGGTGGCCTTTTGGAGTACGCCCGCTCCCATGGCTTT
GCTGCTGAATTCAAGTTGGTCGACCAGTCCGGACCTCCTCACGAGCCCAA
GTTCGTTTACCAAGCAAAAGTTGGGGGTCGCTGGTTCCCAGCCGTCTGCG
CACACAGCAAGAAGCAAGGCAAGCAGGAAGCAGCAGATGCGGCTCTCCGT
GTCTTGATTGGGGAGAACGAGAAGGCAGAACGCATGGGTTTCACAGAGCT
CCCTCTCACTGGCAGCACCTTCCATGACCAGATAGCCATGCTGAGCCACC
GGTGCTTCAACACTCTGACTAACAGCTTCCAGCCCTCCTTGCTCGGCCGC
AAGATTCTGGCCGCCATCATTATGAAAAAAGACTCTGAGGACATGGGTGT
CGTCGTCAGCTTGGGAACAGGGAATCGCTGTGTGAAAGGAGATTCTCTCA
GCCTAAAAGGAGAAACTGTCAATGACTGCCATGCAGAAATAATCTCCCGG
AGAGGCTTCATCAGGTTTCTCTACAGTGAGTTAATGAAATACAACTCCCA
GACTGCGAAGGATAGTATATTTGAACCTGCTAAGGGAGGAGAAAAGCTCC
AAATAAAAAAGACTGTGTCATTCCATCTGTATATCAGCACTGCTCCGTGT
GGAGATGGCGCCCTCTTTGACAAGTCCTGCAGCGACCGTGCTATGGAAAG
CACAGAATCCCGCCACTACCCTGTCTTCGAGAATCCCAAACAAGGAAAGC
TCCGCACCAAGGTGGAGAACGGAGAAGGCACAATCCCTGTGGAATCCAGT
GACATTGTGCCTACGTGGGATGGCATTCGGCTCGGGGAGAGACTCCGTAC
CATGTCCTGTAGTGACAAAATCCTACGCTGGAACGTGCTGGGCCTGCAAG
GGGCACTGTTGACCCACTTCCTGCAGCCCATTTATCTCAAATCTGTCACA
TTGGGTTACCTTTTCAGCCAAGGGCATCTGACCCGTGCTATTTGCTGTCG
TGTGACAAGAGATGGGAGTGCATTTGAGGATGGACTACGACATCCCTTTA
TTGTCAACCACCCCAAGGTTGGCAGAGTCAGCATATATGATTCCAAAAGG
CAATCCGGGAAGACTAAGGAGACAAGCGTCAACTGGTGTCTGGCTGATGG
CTATGACCTGGAGATCCTGGACGGTACCAGAGGCACTGTGGATGGGCCAC
GGAATGAATTGTCCCGGGTCTCCAAAAAGAACATTTTTCTTCTATTTAAG
AAGCTCTGCTCCTTCCGTTACCGCAGGGATCTACTGAGACTCTCCTATGG
TGAGGCCAAGAAAGCTGCCCGTGACTACGAGACGGCCAAGAACTACTTCA
AAAAAGGCCTGAAGGATATGGGCTATGGGAACTGGATTAGCAAACCCCAG
GAGGAAAAGAACTTTTATCTCTGCCCAGTATAGTATGCTCCAGTGACAGA
TGGATTAGGGTGTGTCATACTAGGGTGTGAGAGAGGTAGGTCGTAGCATT
CCTCATCACATGGTCAGGGGATTTTTTTTTCTCCTTTTTTTTTCTTTTTA
AGCCATAATTGGTGATACTGAAAACTTTGGGTTCCCATTTATCCTGCTTT
CTTTGGGATTGCTAGGCAAGGTCTGGCCAGGCCCCCCTTTTTTCCCCCAA
GTGAAGAGGCAGAAACCTAAGAAGTTATCTTTTCTTTCTACCCAAAGCAT
ACATAGTCACTGAGCACCTGCGGTCCATTTCCTCTTAAAAGTTTTGTTTT
GATTTGTTTCCATTTCCTTTCCCTTTGTGTTTGCTACACTGACCTCTTGC
GGTCTTGATTAGGTTTCAGTCAACTCTGGATCATGTCAGGGACTGATAAT
TTCATTTGTGGATTACGCAGACCCCTCTACTTCCCCTCTTTCCCTTCTGA
GATTCTTTCCTTGTGATCTGAATGTCTCCTTTTCCCCCTCAGAGGGCAAA
GAGGTGAACATAAAGGATTTGGTGAAACATTTGTAAGGGTAGGAGTTGAA
AACTGCAGTTCCCAGTGCCACGGAAGTGTGATTGGAGCCTGCAGATAATG
CCCAGCCATCCTCCCATCCTGCACTTTAGCCAGCTGCAGGGCGGGCAAGG
CAAGGAAAGCTGCTTCCCTGGAAGTGTATCACTTTCTCCGGCAGCTGGGA
AGTCTAGAACCAGCCAGACTGGGTTAAGGGAGCTGCTCAAGCAATAGCAG
AGGTTTCACCCGGCAGGATGACACAGACCACTTCCCAGGGAGCACGGGCA
TGCCTTGGAATATTGCCAAGCTTCCAGCTGCCTCTTCTCCTAAAGCATTC
CTAGGAATATTTTCCCCGCCAATGCTGGGCGTACACCCTAGCCAACGGGA
CAAATCCTAGAGGGTATAAAATCATCTCTGCTCAGATAATCATGACTTAG
CAAGAATAAGGGCAAAAAATCCTGTTGGCTTAACGTCACTGTTCCACCCG
GTGTAATATCTCTCATGACAGTGACACCAAGGGAAGTTGACTAAGTCACA
TGTAAATTAGGAGTGTTTTAAAGAATGCCATAGATGTTGATTCTTAACTG
CTACAGATAACCTGTAATTGAGCAGATTTAAAATTCAGGCATACTTTTCC
ATTTATCCAAGTGCTTTCATTTTTCCAGATGGCTTCAGAAGTAGGCTCGT
GGGCAGGGCGCAGACCTGATCTTTATAGGGTTGACATAGAAAGCAGTAGT
TGTGGGTGAAAGGGCAGGTTGTCTTCAAACTCTGTGAGGTAGAATCCTTT
GTCTATACCTCCATGAACATTGACTCGTGTGTTCAGAGCCTTTGGCCTCT
CTGTGGAGTCTGGCTCTCTGGCTCCTGTGCATTCTTTGAATAGTCACTCG
TAAAAACTGTCAGTGCTTGAAACTGTTTCCTTTACTCATGTTGAAGGGAC
TTTGTTGGCTTTTAGAGTGTTGGTCATGACTCCAAGAGCAGAGCAGGGAA
GAGCCCAAGCATAGACTTGGTGCCGTGGTGATGGCTGCAGTCCAGTTTTG
TGATGCTGCTTTTACGTGTCCCTCGATAACAGTCAGCTAGACACACTCAG
GAGGACTACTGAGGCTCTGCGACCTTCAGGAGCTGAGCCTGCCTCTCTCC
TTTAGATGACAGACCTTCATCTGGGAACGTGCTGAGCCAGCACCCTCAGA
TGATTTCCCTCCAAACTGCTGACTAGGTCATCCTCTGTCTGGTAGAGACA
TTCACATCTTTGCTTTTATTCTATGCTCTCTGTACTTTTGACCAAAAATT
GACCAAAGTAAGAAAATGCAAGTTCTAAAAATAGACTAAGGATGCCTTTG
CAGAACACCAAAGCATCCCAAGGAACTGGTAGGGAAGTGGCGCCTGTCTC
CTGGAGTGGAAGAGGCCTGCTCCCTGGCTCTGGGTCTGCTGGGGGCACAG
TAAATCAGTCTTGGCACCCACATCCAGGGCAGAGAGGTCTGTGGTTCTCA
GCATCAGAAGGCAGCGCAGCCCCTCTCCTCTTCAGGCTACAGGGTTGTCA
CCTGCTGAGTCCTCAGGTTGTTTGGCCTCTCTGGTCCATCTTGGGCATTA
GGTTCTCCAGCAGAGCTCTGGCCAGCTGCCTCTTCTTTAACTGGGAACAC
AGGCTCTCACAAGATCAGAACCCCCACTCACCCCCAAGATCTTATCTAGC
AAGCCTGTAGTATTCAGTTTCTGTTGTAGGAAGAGAGCGAGGCATCCCTG
AATTCCACGCATCTGCTGGAAACGAGCCGTGTCAGATCGCACATCCCTGC
GCCCCCATGCCCCTCTGAGTCACACAGGACAGAGGAGGCAGAGCTTCTGC
CCACTGTTATCTTCACTTTCTTTGTCCAGTCTTTTGTTTTTAATAAGCAG
TGACCCTCCCTACTCTTCTTTTTAATGATTTTTGTAGTTGATTTGTCTGA
ACTGTGGCTACTGTGCATTCCTTGAATAATCACTTGTAAAAATTGTCAGT
GCTTGAAGCTGTTTCCTTTACTCACATTGAAGGGACTTCGTTGGTTTTTT
GGAGTCTTGGTTGTGACTCCAAGAGCAGAGTGAGGAAGACCCCCAAGCAT
AGACTCGGGTACTGTGATGATGGCTGCAGTCCAGTTTTATGATTCTGCTT
TTATGTGTCCCTTGATAACAGTGACTTAACAATATACATTCCTCATAAAT
AAAAAAAAAACAAGAATCTGAATTCTTAGAAA

SEQ ID NO: 8 - Human ADAR1 - Transcript Variant 2 Coding Sequence

ATGAATCCGCGGCAGGGGTATTCCCTCAGCGGATACTACACCCATCCATT
TCAAGGCTATGAGCACAGACAGCTCAGGTACCAGCAGCCTGGGCCAGGAT
CTTCCCCCAGTAGTTTCCTGCTTAAGCAAATAGAATTTCTCAAGGGGCAG
CTCCCAGAAGCACCGGTGATTGGAAAGCAGACACCGTCACTGCCACCTTC
CCTCCCAGGACTCCGGCCAAGGTTTCCAGTACTACTTGCCTCCAGTACCA
GAGGCAGGCAAGTGGACATCAGGGGTGTCCCCAGGGGCGTGCATCTCAGA
AGTCAGGGGCTCCAGAGAGGGTTCCAGCATCCTTCACCACGTGGCAGGAG
TCTGCCACAGAGAGGTGTTGATTGCCTTTCCTCACATTTCCAGGAACTGA
GTATCTACCAAGATCAGGAACAAAGGATCTTAAAGTTCCTGGAAGAGCTT
GGGGAAGGGAAGGCCACCACAGCACATGATCTGTCTGGGAAACTTGGGAC
TCCGAAGAAAGAAATCAATCGAGTTTTATACTCCCTGGCAAAGAAGGGCA
AGCTACAGAAAGAGGCAGGAACACCCCCTTTGTGGAAAATCGCGGTCTCC
ACTCAGGCTTGGAACCAGCACAGCGGAGTGGTAAGACCAGACGGTCATAG
CCAAGGAGCCCCAAACTCAGACCCGAGTTTGGAACCGGAAGACAGAAACT
CCACATCTGTCTCAGAAGATCTTCTTGAGCCTTTTATTGCAGTCTCAGCT
CAGGCTTGGAACCAGCACAGCGGAGTGGTAAGACCAGACAGTCATAGCCA
AGGATCCCCAAACTCAGACCCAGGTTTGGAACCTGAAGACAGCAACTCCA
CATCTGCCTTGGAAGATCCTCTTGAGTTTTTAGACATGGCCGAGATCAAG
GAGAAAATCTGCGACTATCTCTTCAATGTGTCTGACTCCTCTGCCCTGAA
TTTGGCTAAAAATATTGGCCTTACCAAGGCCCGAGATATAAATGCTGTGC
TAATTGACATGGAAAGGCAGGGGGATGTCTATAGACAAGGGACAACCCCT
CCCATATGGCATTTGACAGACAAGAAGCGAGAGAGGATGCAAATCAAGAG
AAATACGAACAGTGTTCCTGAAACCGCTCCAGCTGCAATCCCTGAGACCA
AAAGAAACGCAGAGTTCCTCACCTGTAATATACCCACATCAAATGCCTCA
AATAACATGGTAACCACAGAAAAAGTGGAGAATGGGCAGGAACCTGTCAT
AAAGTTAGAAAACAGGCAAGAGGCCAGACCAGAACCAGCAAGACTGAAAC
CACCTGTTCATTACAATGGCCCCTCAAAAGCAGGGTATGTTGACTTTGAA
AATGGCCAGTGGGCCACAGATGACATCCCAGATGACTTGAATAGTATCCG
CGCAGCACCAGGTGAGTTTCGAGCCATCATGGAGATGCCCTCCTTCTACA
GTCATGGCTTGCCACGGTGTTCACCCTACAAGAAACTGACAGAGTGCCAG
CTGAAGAACCCCATCAGCGGGCTGTTAGAATATGCCCAGTTCGCTAGTCA
AACCTGTGAGTTCAACATGATAGAGCAGAGTGGACCACCCCATGAACCTC
GATTTAAATTCCAGGTTGTCATCAATGGCCGAGAGTTTCCCCCAGCTGAA
GCTGGAAGCAAGAAAGTGGCCAAGCAGGATGCAGCTATGAAAGCCATGAC
AATTCTGCTAGAGGAAGCCAAAGCCAAGGACAGTGGAAAATCAGAAGAAT
CATCCCACTATTCCACAGAGAAAGAATCAGAGAAGACTGCAGAGTCCCAG
ACCCCCACCCCTTCAGCCACATCCTTCTTTTCTGGGAAGAGCCCCGTCAC
CACACTGCTTGAGTGTATGCACAAATTGGGGAACTCCTGCGAATTCCGTC
TCCTGTCCAAAGAAGGCCCTGCCCATGAACCCAAGTTCCAATACTGTGTT
GCAGTGGGAGCCCAAACTTTCCCCAGTGTGAGTGCTCCCAGCAAGAAAGT
GGCAAAGCAGATGGCCGCAGAGGAAGCCATGAAGGCCCTGCATGGGGAGG
CGACCAACTCCATGGCTTCTGATAACCAGCCTGAAGGTATGATCTCAGAG
TCACTTGATAACTTGGAATCCATGATGCCCAACAAGGTCAGGAAGATTGG
CGAGCTCGTGAGATACCTGAACACCAACCCTGTGGGTGGCCTTTTGGAGT
ACGCCCGCTCCCATGGCTTTGCTGCTGAATTCAAGTTGGTCGACCAGTCC
GGACCTCCTCACGAGCCCAAGTTCGTTTACCAAGCAAAAGTTGGGGGTCG
CTGGTTCCCAGCCGTCTGCGCACACAGCAAGAAGCAAGGCAAGCAGGAAG
CAGCAGATGCGGCTCTCCGTGTCTTGATTGGGGAGAACGAGAAGGCAGAA
CGCATGGGTTTCACAGAGCTCCCTCTCACTGGCAGCACCTTCCATGACCA
GATAGCCATGCTGAGCCACCGGTGCTTCAACACTCTGACTAACAGCTTCC
AGCCCTCCTTGCTCGGCCGCAAGATTCTGGCCGCCATCATTATGAAAAAA
GACTCTGAGGACATGGGTGTCGTCGTCAGCTTGGGAACAGGGAATCGCTG
TGTGAAAGGAGATTCTCTCAGCCTAAAAGGAGAAACTGTCAATGACTGCC
ATGCAGAAATAATCTCCCGGAGAGGCTTCATCAGGTTTCTCTACAGTGAG
TTAATGAAATACAACTCCCAGACTGCGAAGGATAGTATATTTGAACCTGC
TAAGGGAGGAGAAAAGCTCCAAATAAAAAAGACTGTGTCATTCCATCTGT
ATATCAGCACTGCTCCGTGTGGAGATGGCGCCCTCTTTGACAAGTCCTGC
AGCGACCGTGCTATGGAAAGCACAGAATCCCGCCACTACCCTGTCTTCGA
GAATCCCAAACAAGGAAAGCTCCGCACCAAGGTGGAGAACGGAGAAGGCA
CAATCCCTGTGGAATCCAGTGACATTGTGCCTACGTGGGATGGCATTCGG
CTCGGGGAGAGACTCCGTACCATGTCCTGTAGTGACAAAATCCTACGCTG
GAACGTGCTGGGCCTGCAAGGGGCACTGTTGACCCACTTCCTGCAGCCCA
TTTATCTCAAATCTGTCACATTGGGTTACCTTTTCAGCCAAGGGCATCTG
ACCCGTGCTATTTGCTGTCGTGTGACAAGAGATGGGAGTGCATTTGAGGA
TGGACTACGACATCCCTTTATTGTCAACCACCCCAAGGTTGGCAGAGTCA
GCATATATGATTCCAAAAGGCAATCCGGGAAGACTAAGGAGACAAGCGTC
AACTGGTGTCTGGCTGATGGCTATGACCTGGAGATCCTGGACGGTACCAG
AGGCACTGTGGATGGGCCACGGAATGAATTGTCCCGGGTCTCCAAAAAGA
ACATTTTTCTTCTATTTAAGAAGCTCTGCTCCTTCCGTTACCGCAGGGAT
CTACTGAGACTCTCCTATGGTGAGGCCAAGAAAGCTGCCCGTGACTACGA
GACGGCCAAGAACTACTTCAAAAAAGGCCTGAAGGATATGGGCTATGGGA
ACTGGATTAGCAAACCCCAGGAGGAAAAGAACTTTTATCTCTGCCCAGTA
TAG

SEQ ID NO: 9 - Human ADAR1 - Transcript Variant 2 Amino Acid Sequence (aka isoform-b)

MNPRQGYSLSGYYTHPFQGYEHRQLRYQQPGPGSSPSSFLLKQIEFLKGQ
LPEAPVIGKQTPSLPPSLPGLRPRFPVLLASSTRGRQVDIRGVPRGVHLR
SQGLQRGFQHPSPRGRSLPQRGVDCLSSHFQELSIYQDQEQRILKFLEEL
GEGKATTAHDLSGKLGTPKKEINRVLYSLAKKGKLQKEAGTPPLWKIAVS
TQAWNQHSGVVRPDGHSQGAPNSDPSLEPEDRNSTSVSEDLLEPFIAVSA
QAWNQHSGVVRPDSHSQGSPNSDPGLEPEDSNSTSALEDPLEFLDMAEIK
EKICDYLFNVSDSSALNLAKNIGLTKARDINAVLIDMERQGDVYRQGTTP
PIWHLTDKKRERMQIKRNTNSVPETAPAAIPETKRNAEFLTCNIPTSNAS
NNMVTTEKVENGQEPVIKLENRQEARPEPARLKPPVHYNGPSKAGYVDFE
NGQWATDDIPDDLNSIRAAPGEFRAIMEMPSFYSHGLPRCSPYKKLTECQ
LKNPISGLLEYAQFASQTCEFNMIEQSGPPHEPRFKFQVVINGREFPPAE
AGSKKVAKQDAAMKAMTILLEEAKAKDSGKSEESSHYSTEKESEKTAESQ
TPTPSATSFFSGKSPVTTLLECMHKLGNSCEFRLLSKEGPAHEPKFQYCV
AVGAQTFPSVSAPSKKVAKQMAAEEAMKALHGEATNSMASDNQPEGMISE
SLDNLESMMPNKVRKIGELVRYLNTNPVGGLLEYARSHGFAAEFKLVDQS
GPPHEPKFVYQAKVGGRWFPAVCAHSKKQGKQEAADAALRVLIGENEKAE
RMGFTELPLTGSTFHDQIAMLSHRCFNTLTNSFQPSLLGRKILAAIIMKK
DSEDMGVVVSLGTGNRCVKGDSLSLKGETVNDCHAEIISRRGFIRFLYSE
LMKYNSQTAKDSIFEPAKGGEKLQIKKTVSFHLYISTAPCGDGALFDKSC
SDRAMESTESRHYPVFENPKQGKLRTKVENGEGTIPVESSDIVPTWDGIR
LGERLRTMSCSDKILRWNVLGLQGALLTHFLQPIYLKSVTLGYLFSQGHL
TRAICCRVTRDGSAFEDGLRHPFIVNHPKVGRVSIYDSKRQSGKTKETSV
NWCLADGYDLEILDGTRGTVDGPRNELSRVSKKNIFLLFKKLCSFRYRRD
LLRLSYGEAKKAARDYETAKNYFKKGLKDMGYGNWISKPQEEKNFYLCPV

In certain embodiments, an ADAR1 polynucleotide or a human ADAR1 gene incorporated into a non-human animal (e.g., a rodent, e.g., a rat or mouse) is represented by or comprises a sequence encoding a human ADAR1 transcript variant 3 or a characteristic portion thereof. In certain embodiments, a ADAR1 polynucleotide or a human ADAR1 gene comprises, or consists of, a nucleotide sequence that is the same as, differs by no more than 1, 2, 3, 4, 5, 6, 7, 8, 9, or 10 nucleotides from, or has at least has at least 90%, 92%, 94%, 96%, 98%, or 99% homology with, the nucleotide sequence of SEQ ID NO: 10 or SEQ ID NO: 11 or a characteristic portion thereof. In some embodiments, a human ADAR1 gene comprises, or consists of, a nucleotide sequence that is the same as the nucleotide sequence of SEQ ID NO: 10 or SEQ ID NO: 11 or a characteristic portion thereof. In certain embodiments, an encoded human ADAR1 Amino Acid sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the Amino Acid sequence of SEQ ID NO: 12 or a characteristic portion thereof. In certain embodiments, the amino acid sequence of an encoded and/or expressed ADAR1 polypeptide or a characteristic portion thereof, is or comprises a sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the amino acid sequence of SEQ ID NO: 12 or a characteristic portion thereof.

SEQ ID NO: 10 - Human ADAR1 - Transcript Variant 3 cDNA Sequence

GAACCGGAGCCATCTTGGGCCCGGCGCGCAGACCCGCGGAGTTTCCCGTG
CCGACGCCCCGGGGCCACTTCCAGTGCGGAGTAGCGGAGGCGTGGGGGCC
TCGAGGGGCTGGCGCGGCCCAGCGGTCGGGCCAGGGTCGTGCCGCCGGCG
GGTCGGGCCGGGCAATGCCTCGCGGGCGCAATGAATCCGCGGCAGGGGTA
TTCCCTCAGCGGATACTACACCCATCCATTTCAAGGCTATGAGCACAGAC
AGCTCAGGTACCAGCAGCCTGGGCCAGGATCTTCCCCCAGTAGTTTCCTG
CTTAAGCAAATAGAATTTCTCAAGGGGCAGCTCCCAGAAGCACCGGTGAT
TGGAAAGCAGACACCGTCACTGCCACCTTCCCTCCCAGGACTCCGGCCAA
GGTTTCCAGTACTACTTGCCTCCAGTACCAGAGGCAGGCAAGTGGACATC
AGGGGTGTCCCCAGGGGCGTGCATCTCAGAAGTCAGGGGCTCCAGAGAGG
GTTCCAGCATCCTTCACCACGTGGCAGGAGTCTGCCACAGAGAGGTGTTG
ATTGCCTTTCCTCACATTTCCAGGAACTGAGTATCTACCAAGATCAGGAA
CAAAGGATCTTAAAGTTCCTGGAAGAGCTTGGGGAAGGGAAGGCCACCAC
AGCACATGATCTGTCTGGGAAACTTGGGACTCCGAAGAAAGAAATCAATC
GAGTTTTATACTCCCTGGCAAAGAAGGGCAAGCTACAGAAAGAGGCAGGA
ACACCCCCTTTGTGGAAAATCGCGGTCTCCACTCAGGCTTGGAACCAGCA
CAGCGGAGTGGTAAGACCAGACGGTCATAGCCAAGGAGCCCCAAACTCAG
ACCCGAGTTTGGAACCGGAAGACAGAAACTCCACATCTGTCTCAGAAGAT
CTTCTTGAGCCTTTTATTGCAGTCTCAGCTCAGGCTTGGAACCAGCACAG
CGGAGTGGTAAGACCAGACAGTCATAGCCAAGGATCCCCAAACTCAGACC
CAGGTTTGGAACCTGAAGACAGCAACTCCACATCTGCCTTGGAAGATCCT
CTTGAGTTTTTAGACATGGCCGAGATCAAGGAGAAAATCTGCGACTATCT
CTTCAATGTGTCTGACTCCTCTGCCCTGAATTTGGCTAAAAATATTGGCC
TTACCAAGGCCCGAGATATAAATGCTGTGCTAATTGACATGGAAAGGCAG
GGGGATGTCTATAGACAAGGGACAACCCCTCCCATATGGCATTTGACAGA
CAAGAAGCGAGAGAGGATGCAAATCAAGAGAAATACGAACAGTGTTCCTG
AAACCGCTCCAGCTGCAATCCCTGAGACCAAAAGAAACGCAGAGTTCCTC
ACCTGTAATATACCCACATCAAATGCCTCAAATAACATGGTAACCACAGA
AAAAGTGGAGAATGGGCAGGAACCTGTCATAAAGTTAGAAAACAGGCAAG
AGGCCAGACCAGAACCAGCAAGACTGAAACCACCTGTTCATTACAATGGC
CCCTCAAAAGCAGGGTATGTTGACTTTGAAAATGGCCAGTGGGCCACAGA
TGACATCCCAGATGACTTGAATAGTATCCGCGCAGCACCAGGTGAGTTTC
GAGCCATCATGGAGATGCCCTCCTTCTACAGTCATGGCTTGCCACGGTGT
TCACCCTACAAGAAACTGACAGAGTGCCAGCTGAAGAACCCCATCAGCGG
GCTGTTAGAATATGCCCAGTTCGCTAGTCAAACCTGTGAGTTCAACATGA
TAGAGCAGAGTGGACCACCCCATGAACCTCGATTTAAATTCCAGGTTGTC
ATCAATGGCCGAGAGTTTCCCCCAGCTGAAGCTGGAAGCAAGAAAGTGGC
CAAGCAGGATGCAGCTATGAAAGCCATGACAATTCTGCTAGAGGAAGCCA
AAGCCAAGGACAGTGGAAAATCAGAAGAATCATCCCACTATTCCACAGAG
AAAGAATCAGAGAAGACTGCAGAGTCCCAGACCCCCACCCCTTCAGCCAC
ATCCTTCTTTTCTGGGAAGAGCCCCGTCACCACACTGCTTGAGTGTATGC
ACAAATTGGGGAACTCCTGCGAATTCCGTCTCCTGTCCAAAGAAGGCCCT
GCCCATGAACCCAAGTTCCAATACTGTGTTGCAGTGGGAGCCCAAACTTT
CCCCAGTGTGAGTGCTCCCAGCAAGAAAGTGGCAAAGCAGATGGCCGCAG
AGGAAGCCATGAAGGCCCTGCATGGGGAGGCGACCAACTCCATGGCTTCT
GATAACCAGGTCAGGAAGATTGGCGAGCTCGTGAGATACCTGAACACCAA
CCCTGTGGGTGGCCTTTTGGAGTACGCCCGCTCCCATGGCTTTGCTGCTG
AATTCAAGTTGGTCGACCAGTCCGGACCTCCTCACGAGCCCAAGTTCGTT
TACCAAGCAAAAGTTGGGGGTCGCTGGTTCCCAGCCGTCTGCGCACACAG
CAAGAAGCAAGGCAAGCAGGAAGCAGCAGATGCGGCTCTCCGTGTCTTGA
TTGGGGAGAACGAGAAGGCAGAACGCATGGGTTTCACAGAGCTCCCTCTC
ACTGGCAGCACCTTCCATGACCAGATAGCCATGCTGAGCCACCGGTGCTT
CAACACTCTGACTAACAGCTTCCAGCCCTCCTTGCTCGGCCGCAAGATTC
TGGCCGCCATCATTATGAAAAAAGACTCTGAGGACATGGGTGTCGTCGTC
AGCTTGGGAACAGGGAATCGCTGTGTGAAAGGAGATTCTCTCAGCCTAAA
AGGAGAAACTGTCAATGACTGCCATGCAGAAATAATCTCCCGGAGAGGCT
TCATCAGGTTTCTCTACAGTGAGTTAATGAAATACAACTCCCAGACTGCG
AAGGATAGTATATTTGAACCTGCTAAGGGAGGAGAAAAGCTCCAAATAAA
AAAGACTGTGTCATTCCATCTGTATATCAGCACTGCTCCGTGTGGAGATG
GCGCCCTCTTTGACAAGTCCTGCAGCGACCGTGCTATGGAAAGCACAGAA
TCCCGCCACTACCCTGTCTTCGAGAATCCCAAACAAGGAAAGCTCCGCAC
CAAGGTGGAGAACGGAGAAGGCACAATCCCTGTGGAATCCAGTGACATTG
TGCCTACGTGGGATGGCATTCGGCTCGGGGAGAGACTCCGTACCATGTCC
TGTAGTGACAAAATCCTACGCTGGAACGTGCTGGGCCTGCAAGGGGCACT
GTTGACCCACTTCCTGCAGCCCATTTATCTCAAATCTGTCACATTGGGTT
ACCTTTTCAGCCAAGGGCATCTGACCCGTGCTATTTGCTGTCGTGTGACA
AGAGATGGGAGTGCATTTGAGGATGGACTACGACATCCCTTTATTGTCAA
CCACCCCAAGGTTGGCAGAGTCAGCATATATGATTCCAAAAGGCAATCCG
GGAAGACTAAGGAGACAAGCGTCAACTGGTGTCTGGCTGATGGCTATGAC
CTGGAGATCCTGGACGGTACCAGAGGCACTGTGGATGGGCCACGGAATGA
ATTGTCCCGGGTCTCCAAAAAGAACATTTTTCTTCTATTTAAGAAGCTCT
GCTCCTTCCGTTACCGCAGGGATCTACTGAGACTCTCCTATGGTGAGGCC
AAGAAAGCTGCCCGTGACTACGAGACGGCCAAGAACTACTTCAAAAAAGG
CCTGAAGGATATGGGCTATGGGAACTGGATTAGCAAACCCCAGGAGGAAA
AGAACTTTTATCTCTGCCCAGTATAGTATGCTCCAGTGACAGATGGATTA
GGGTGTGTCATACTAGGGTGTGAGAGAGGTAGGTCGTAGCATTCCTCATC
ACATGGTCAGGGGATTTTTTTTTCTCCTTTTTTTTTCTTTTTAAGCCATA
ATTGGTGATACTGAAAACTTTGGGTTCCCATTTATCCTGCTTTCTTTGGG
ATTGCTAGGCAAGGTCTGGCCAGGCCCCCCTTTTTTCCCCCAAGTGAAGA
GGCAGAAACCTAAGAAGTTATCTTTTCTTTCTACCCAAAGCATACATAGT
CACTGAGCACCTGCGGTCCATTTCCTCTTAAAAGTTTTGTTTTGATTTGT
TTCCATTTCCTTTCCCTTTGTGTTTGCTACACTGACCTCTTGCGGTCTTG
ATTAGGTTTCAGTCAACTCTGGATCATGTCAGGGACTGATAATTTCATTT
GTGGATTACGCAGACCCCTCTACTTCCCCTCTTTCCCTTCTGAGATTCTT
TCCTTGTGATCTGAATGTCTCCTTTTCCCCCTCAGAGGGCAAAGAGGTGA
ACATAAAGGATTTGGTGAAACATTTGTAAGGGTAGGAGTTGAAAACTGCA
GTTCCCAGTGCCACGGAAGTGTGATTGGAGCCTGCAGATAATGCCCAGCC
ATCCTCCCATCCTGCACTTTAGCCAGCTGCAGGGCGGGCAAGGCAAGGAA
AGCTGCTTCCCTGGAAGTGTATCACTTTCTCCGGCAGCTGGGAAGTCTAG
AACCAGCCAGACTGGGTTAAGGGAGCTGCTCAAGCAATAGCAGAGGTTTC
ACCCGGCAGGATGACACAGACCACTTCCCAGGGAGCACGGGCATGCCTTG
GAATATTGCCAAGCTTCCAGCTGCCTCTTCTCCTAAAGCATTCCTAGGAA
TATTTTCCCCGCCAATGCTGGGCGTACACCCTAGCCAACGGGACAAATCC
TAGAGGGTATAAAATCATCTCTGCTCAGATAATCATGACTTAGCAAGAAT
AAGGGCAAAAAATCCTGTTGGCTTAACGTCACTGTTCCACCCGGTGTAAT
ATCTCTCATGACAGTGACACCAAGGGAAGTTGACTAAGTCACATGTAAAT
TAGGAGTGTTTTAAAGAATGCCATAGATGTTGATTCTTAACTGCTACAGA
TAACCTGTAATTGAGCAGATTTAAAATTCAGGCATACTTTTCCATTTATC
CAAGTGCTTTCATTTTTCCAGATGGCTTCAGAAGTAGGCTCGTGGGCAGG
GCGCAGACCTGATCTTTATAGGGTTGACATAGAAAGCAGTAGTTGTGGGT
GAAAGGGCAGGTTGTCTTCAAACTCTGTGAGGTAGAATCCTTTGTCTATA
CCTCCATGAACATTGACTCGTGTGTTCAGAGCCTTTGGCCTCTCTGTGGA
GTCTGGCTCTCTGGCTCCTGTGCATTCTTTGAATAGTCACTCGTAAAAAC
TGTCAGTGCTTGAAACTGTTTCCTTTACTCATGTTGAAGGGACTTTGTTG
GCTTTTAGAGTGTTGGTCATGACTCCAAGAGCAGAGCAGGGAAGAGCCCA
AGCATAGACTTGGTGCCGTGGTGATGGCTGCAGTCCAGTTTTGTGATGCT
GCTTTTACGTGTCCCTCGATAACAGTCAGCTAGACACACTCAGGAGGACT
ACTGAGGCTCTGCGACCTTCAGGAGCTGAGCCTGCCTCTCTCCTTTAGAT
GACAGACCTTCATCTGGGAACGTGCTGAGCCAGCACCCTCAGATGATTTC
CCTCCAAACTGCTGACTAGGTCATCCTCTGTCTGGTAGAGACATTCACAT
CTTTGCTTTTATTCTATGCTCTCTGTACTTTTGACCAAAAATTGACCAAA
GTAAGAAAATGCAAGTTCTAAAAATAGACTAAGGATGCCTTTGCAGAACA
CCAAAGCATCCCAAGGAACTGGTAGGGAAGTGGCGCCTGTCTCCTGGAGT
GGAAGAGGCCTGCTCCCTGGCTCTGGGTCTGCTGGGGGCACAGTAAATCA
GTCTTGGCACCCACATCCAGGGCAGAGAGGTCTGTGGTTCTCAGCATCAG
AAGGCAGCGCAGCCCCTCTCCTCTTCAGGCTACAGGGTTGTCACCTGCTG
AGTCCTCAGGTTGTTTGGCCTCTCTGGTCCATCTTGGGCATTAGGTTCTC
CAGCAGAGCTCTGGCCAGCTGCCTCTTCTTTAACTGGGAACACAGGCTCT
CACAAGATCAGAACCCCCACTCACCCCCAAGATCTTATCTAGCAAGCCTG
TAGTATTCAGTTTCTGTTGTAGGAAGAGAGCGAGGCATCCCTGAATTCCA
CGCATCTGCTGGAAACGAGCCGTGTCAGATCGCACATCCCTGCGCCCCCA
TGCCCCTCTGAGTCACACAGGACAGAGGAGGCAGAGCTTCTGCCCACTGT
TATCTTCACTTTCTTTGTCCAGTCTTTTGTTTTTAATAAGCAGTGACCCT
CCCTACTCTTCTTTTTAATGATTTTTGTAGTTGATTTGTCTGAACTGTGG
CTACTGTGCATTCCTTGAATAATCACTTGTAAAAATTGTCAGTGCTTGAA
GCTGTTTCCTTTACTCACATTGAAGGGACTTCGTTGGTTTTTTGGAGTCT
TGGTTGTGACTCCAAGAGCAGAGTGAGGAAGACCCCCAAGCATAGACTCG
GGTACTGTGATGATGGCTGCAGTCCAGTTTTATGATTCTGCTTTTATGTG
TCCCTTGATAACAGTGACTTAACAATATACATTCCTCATAAATAAAAAAA
AAACAAGAATCTGAATTCTTAGAAA

SEQ ID NO: 11 - Human ADAR1 - Transcript Variant 3 Coding Sequence

ATGAATCCGCGGCAGGGGTATTCCCTCAGCGGATACTACACCCATCCATT
TCAAGGCTATGAGCACAGACAGCTCAGGTACCAGCAGCCTGGGCCAGGAT
CTTCCCCCAGTAGTTTCCTGCTTAAGCAAATAGAATTTCTCAAGGGGCAG
CTCCCAGAAGCACCGGTGATTGGAAAGCAGACACCGTCACTGCCACCTTC
CCTCCCAGGACTCCGGCCAAGGTTTCCAGTACTACTTGCCTCCAGTACCA
GAGGCAGGCAAGTGGACATCAGGGGTGTCCCCAGGGGCGTGCATCTCAGA
AGTCAGGGGCTCCAGAGAGGGTTCCAGCATCCTTCACCACGTGGCAGGAG
TCTGCCACAGAGAGGTGTTGATTGCCTTTCCTCACATTTCCAGGAACTGA
GTATCTACCAAGATCAGGAACAAAGGATCTTAAAGTTCCTGGAAGAGCTT
GGGGAAGGGAAGGCCACCACAGCACATGATCTGTCTGGGAAACTTGGGAC
TCCGAAGAAAGAAATCAATCGAGTTTTATACTCCCTGGCAAAGAAGGGCA
AGCTACAGAAAGAGGCAGGAACACCCCCTTTGTGGAAAATCGCGGTCTCC
ACTCAGGCTTGGAACCAGCACAGCGGAGTGGTAAGACCAGACGGTCATAG
CCAAGGAGCCCCAAACTCAGACCCGAGTTTGGAACCGGAAGACAGAAACT
CCACATCTGTCTCAGAAGATCTTCTTGAGCCTTTTATTGCAGTCTCAGCT
CAGGCTTGGAACCAGCACAGCGGAGTGGTAAGACCAGACAGTCATAGCCA
AGGATCCCCAAACTCAGACCCAGGTTTGGAACCTGAAGACAGCAACTCCA
CATCTGCCTTGGAAGATCCTCTTGAGTTTTTAGACATGGCCGAGATCAAG
GAGAAAATCTGCGACTATCTCTTCAATGTGTCTGACTCCTCTGCCCTGAA
TTTGGCTAAAAATATTGGCCTTACCAAGGCCCGAGATATAAATGCTGTGC
TAATTGACATGGAAAGGCAGGGGGATGTCTATAGACAAGGGACAACCCCT
CCCATATGGCATTTGACAGACAAGAAGCGAGAGAGGATGCAAATCAAGAG
AAATACGAACAGTGTTCCTGAAACCGCTCCAGCTGCAATCCCTGAGACCA
AAAGAAACGCAGAGTTCCTCACCTGTAATATACCCACATCAAATGCCTCA
AATAACATGGTAACCACAGAAAAAGTGGAGAATGGGCAGGAACCTGTCAT
AAAGTTAGAAAACAGGCAAGAGGCCAGACCAGAACCAGCAAGACTGAAAC
CACCTGTTCATTACAATGGCCCCTCAAAAGCAGGGTATGTTGACTTTGAA
AATGGCCAGTGGGCCACAGATGACATCCCAGATGACTTGAATAGTATCCG
CGCAGCACCAGGTGAGTTTCGAGCCATCATGGAGATGCCCTCCTTCTACA
GTCATGGCTTGCCACGGTGTTCACCCTACAAGAAACTGACAGAGTGCCAG
CTGAAGAACCCCATCAGCGGGCTGTTAGAATATGCCCAGTTCGCTAGTCA
AACCTGTGAGTTCAACATGATAGAGCAGAGTGGACCACCCCATGAACCTC
GATTTAAATTCCAGGTTGTCATCAATGGCCGAGAGTTTCCCCCAGCTGAA
GCTGGAAGCAAGAAAGTGGCCAAGCAGGATGCAGCTATGAAAGCCATGAC
AATTCTGCTAGAGGAAGCCAAAGCCAAGGACAGTGGAAAATCAGAAGAAT
CATCCCACTATTCCACAGAGAAAGAATCAGAGAAGACTGCAGAGTCCCAG
ACCCCCACCCCTTCAGCCACATCCTTCTTTTCTGGGAAGAGCCCCGTCAC
CACACTGCTTGAGTGTATGCACAAATTGGGGAACTCCTGCGAATTCCGTC
TCCTGTCCAAAGAAGGCCCTGCCCATGAACCCAAGTTCCAATACTGTGTT
GCAGTGGGAGCCCAAACTTTCCCCAGTGTGAGTGCTCCCAGCAAGAAAGT
GGCAAAGCAGATGGCCGCAGAGGAAGCCATGAAGGCCCTGCATGGGGAGG
CGACCAACTCCATGGCTTCTGATAACCAGGTCAGGAAGATTGGCGAGCTC
GTGAGATACCTGAACACCAACCCTGTGGGTGGCCTTTTGGAGTACGCCCG
CTCCCATGGCTTTGCTGCTGAATTCAAGTTGGTCGACCAGTCCGGACCTC
CTCACGAGCCCAAGTTCGTTTACCAAGCAAAAGTTGGGGGTCGCTGGTTC
CCAGCCGTCTGCGCACACAGCAAGAAGCAAGGCAAGCAGGAAGCAGCAGA
TGCGGCTCTCCGTGTCTTGATTGGGGAGAACGAGAAGGCAGAACGCATGG
GTTTCACAGAGCTCCCTCTCACTGGCAGCACCTTCCATGACCAGATAGCC
ATGCTGAGCCACCGGTGCTTCAACACTCTGACTAACAGCTTCCAGCCCTC
CTTGCTCGGCCGCAAGATTCTGGCCGCCATCATTATGAAAAAAGACTCTG
AGGACATGGGTGTCGTCGTCAGCTTGGGAACAGGGAATCGCTGTGTGAAA
GGAGATTCTCTCAGCCTAAAAGGAGAAACTGTCAATGACTGCCATGCAGA
AATAATCTCCCGGAGAGGCTTCATCAGGTTTCTCTACAGTGAGTTAATGA
AATACAACTCCCAGACTGCGAAGGATAGTATATTTGAACCTGCTAAGGGA
GGAGAAAAGCTCCAAATAAAAAAGACTGTGTCATTCCATCTGTATATCAG
CACTGCTCCGTGTGGAGATGGCGCCCTCTTTGACAAGTCCTGCAGCGACC
GTGCTATGGAAAGCACAGAATCCCGCCACTACCCTGTCTTCGAGAATCCC
AAACAAGGAAAGCTCCGCACCAAGGTGGAGAACGGAGAAGGCACAATCCC
TGTGGAATCCAGTGACATTGTGCCTACGTGGGATGGCATTCGGCTCGGGG
AGAGACTCCGTACCATGTCCTGTAGTGACAAAATCCTACGCTGGAACGTG
CTGGGCCTGCAAGGGGCACTGTTGACCCACTTCCTGCAGCCCATTTATCT
CAAATCTGTCACATTGGGTTACCTTTTCAGCCAAGGGCATCTGACCCGTG
CTATTTGCTGTCGTGTGACAAGAGATGGGAGTGCATTTGAGGATGGACTA
CGACATCCCTTTATTGTCAACCACCCCAAGGTTGGCAGAGTCAGCATATA
TGATTCCAAAAGGCAATCCGGGAAGACTAAGGAGACAAGCGTCAACTGGT
GTCTGGCTGATGGCTATGACCTGGAGATCCTGGACGGTACCAGAGGCACT
GTGGATGGGCCACGGAATGAATTGTCCCGGGTCTCCAAAAAGAACATTTT
TCTTCTATTTAAGAAGCTCTGCTCCTTCCGTTACCGCAGGGATCTACTGA
GACTCTCCTATGGTGAGGCCAAGAAAGCTGCCCGTGACTACGAGACGGCC
AAGAACTACTTCAAAAAAGGCCTGAAGGATATGGGCTATGGGAACTGGAT
TAGCAAACCCCAGGAGGAAAAGAACTTTTATCTCTGCCCAGTATAG

SEQ ID NO: 12 - Human ADAR1 - Transcript Variant 3 Amino Acid Sequence

MNPRQGYSLSGYYTHPFQGYEHRQLRYQQPGPGSSPSSFLLKQIEFLKGQ
LPEAPVIGKQTPSLPPSLPGLRPRFPVLLASSTRGRQVDIRGVPRGVHLR
SQGLQRGFQHPSPRGRSLPQRGVDCLSSHFQELSIYQDQEQRILKFLEEL
GEGKATTAHDLSGKLGTPKKEINRVLYSLAKKGKLQKEAGTPPLWKIAVS
TQAWNQHSGVVRPDGHSQGAPNSDPSLEPEDRNSTSVSEDLLEPFIAVSA
QAWNQHSGVVRPDSHSQGSPNSDPGLEPEDSNSTSALEDPLEFLDMAEIK
EKICDYLFNVSDSSALNLAKNIGLTKARDINAVLIDMERQGDVYRQGTTP
PIWHLTDKKRERMQIKRNTNSVPETAPAAIPETKRNAEFLTCNIPTSNAS
NNMVTTEKVENGQEPVIKLENRQEARPEPARLKPPVHYNGPSKAGYVDFE
NGQWATDDIPDDLNSIRAAPGEFRAIMEMPSFYSHGLPRCSPYKKLTECQ
LKNPISGLLEYAQFASQTCEFNMIEQSGPPHEPRFKFQVVINGREFPPAE
AGSKKVAKQDAAMKAMTILLEEAKAKDSGKSEESSHYSTEKESEKTAESQ
TPTPSATSFFSGKSPVTTLLECMHKLGNSCEFRLLSKEGPAHEPKFQYCV
AVGAQTFPSVSAPSKKVAKQMAAEEAMKALHGEATNSMASDNQVRKIGEL
VRYLNTNPVGGLLEYARSHGFAAEFKLVDQSGPPHEPKFVYQAKVGGRWF
PAVCAHSKKQGKQEAADAALRVLIGENEKAERMGFTELPLTGSTFHDQIA
MLSHRCFNTLTNSFQPSLLGRKILAAIIMKKDSEDMGVVVSLGTGNRCVK
GDSLSLKGETVNDCHAEIISRRGFIRFLYSELMKYNSQTAKDSIFEPAKG
GEKLQIKKTVSFHLYISTAPCGDGALFDKSCSDRAMESTESRHYPVFENP
KQGKLRTKVENGEGTIPVESSDIVPTWDGIRLGERLRTMSCSDKILRWNV
LGLQGALLTHFLQPIYLKSVTLGYLFSQGHLTRAICCRVTRDGSAFEDGL
RHPFIVNHPKVGRVSIYDSKRQSGKTKETSVNWCLADGYDLEILDGTRGT
VDGPRNELSRVSKKNIFLLFKKLCSFRYRRDLLRLSYGEAKKAARDYETA
KNYFKKGLKDMGYGNWISKPQEEKNFYLCPV

In certain embodiments, an ADAR1 polynucleotide or a human ADAR1 gene incorporated into a non-human animal (e.g., a rodent, e.g., a rat or mouse) is represented by or comprises a sequence encoding a human ADAR1 transcript variant 4 or a characteristic portion thereof. In certain embodiments, an ADAR1 polynucleotide or a human ADAR1 gene comprises, or consists of, a nucleotide sequence that is the same as, differs by no more than 1, 2, 3, 4, 5, 6, 7, 8, 9, or 10 nucleotides from, or has at least has at least 90%, 92%, 94%, 96%, 98%, or 99% homology with, the nucleotide sequence of SEQ ID NO: 13, SEQ ID NO: 14, or SEQ ID NO: 15 or a characteristic portion thereof. In some embodiments, an ADAR1 polynucleotide or a human ADAR1 gene comprises, or consists of, a nucleotide sequence that is the same as the nucleotide sequence of SEQ ID NO: 13, SEQ ID NO: 14, or SEQ ID NO: 15 or characteristic portion thereof. In certain embodiments, an encoded human ADAR1 Amino Acid sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the Amino Acid sequence of SEQ ID NO: 16 or characteristic portion. In certain embodiments, the amino acid sequence of an encoded and/or expressed ADAR1 polypeptide or a characteristic portion thereof, is or comprises a sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the amino acid sequence of SEQ ID NO: 16 or a characteristic portion thereof.

SEQ ID NO: 13 - Human ADAR1 - Transcript Variant 4 cDNA Sequence

ATTGATTCCCGACTGAAGGTAGAGAAGGCTACGTGGTGGGGGAGGGTGGG
GGGAGGGTCGCGGCCGCACTGGCAGTCTCCGGGTGTCCGGCCGTGTCCCG
AGGAAGTGCAAGACCCGGGGTATTCCCTCAGCGGATACTACACCCATCCA
TTTCAAGGCTATGAGCACAGACAGCTCAGGTACCAGCAGCCTGGGCCAGG
ATCTTCCCCCAGTAGTTTCCTGCTTAAGCAAATAGAATTTCTCAAGGGGC
AGCTCCCAGAAGCACCGGTGATTGGAAAGCAGACACCGTCACTGCCACCT
TCCCTCCCAGGACTCCGGCCAAGGTTTCCAGTACTACTTGCCTCCAGTAC
CAGAGGCAGGCAAGTGGACATCAGGGGTGTCCCCAGGGGCGTGCATCTCA
GAAGTCAGGGGCTCCAGAGAGGGTTCCAGCATCCTTCACCACGTGGCAGG
AGTCTGCCACAGAGAGGTGTTGATTGCCTTTCCTCACATTTCCAGGAACT
GAGTATCTACCAAGATCAGGAACAAAGGATCTTAAAGTTCCTGGAAGAGC
TTGGGGAAGGGAAGGCCACCACAGCACATGATCTGTCTGGGAAACTTGGG
ACTCCGAAGAAAGAAATCAATCGAGTTTTATACTCCCTGGCAAAGAAGGG
CAAGCTACAGAAAGAGGCAGGAACACCCCCTTTGTGGAAAATCGCGGTCT
CCACTCAGGCTTGGAACCAGCACAGCGGAGTGGTAAGACCAGACGGTCAT
AGCCAAGGAGCCCCAAACTCAGACCCGAGTTTGGAACCGGAAGACAGAAA
CTCCACATCTGTCTCAGAAGATCTTCTTGAGCCTTTTATTGCAGTCTCAG
CTCAGGCTTGGAACCAGCACAGCGGAGTGGTAAGACCAGACAGTCATAGC
CAAGGATCCCCAAACTCAGACCCAGGTTTGGAACCTGAAGACAGCAACTC
CACATCTGCCTTGGAAGATCCTCTTGAGTTTTTAGACATGGCCGAGATCA
AGGAGAAAATCTGCGACTATCTCTTCAATGTGTCTGACTCCTCTGCCCTG
AATTTGGCTAAAAATATTGGCCTTACCAAGGCCCGAGATATAAATGCTGT
GCTAATTGACATGGAAAGGCAGGGGGATGTCTATAGACAAGGGACAACCC
CTCCCATATGGCATTTGACAGACAAGAAGCGAGAGAGGATGCAAATCAAG
AGAAATACGAACAGTGTTCCTGAAACCGCTCCAGCTGCAATCCCTGAGAC
CAAAAGAAACGCAGAGTTCCTCACCTGTAATATACCCACATCAAATGCCT
CAAATAACATGGTAACCACAGAAAAAGTGGAGAATGGGCAGGAACCTGTC
ATAAAGTTAGAAAACAGGCAAGAGGCCAGACCAGAACCAGCAAGACTGAA
ACCACCTGTTCATTACAATGGCCCCTCAAAAGCAGGGTATGTTGACTTTG
AAAATGGCCAGTGGGCCACAGATGACATCCCAGATGACTTGAATAGTATC
CGCGCAGCACCAGGTGAGTTTCGAGCCATCATGGAGATGCCCTCCTTCTA
CAGTCATGGCTTGCCACGGTGTTCACCCTACAAGAAACTGACAGAGTGCC
AGCTGAAGAACCCCATCAGCGGGCTGTTAGAATATGCCCAGTTCGCTAGT
CAAACCTGTGAGTTCAACATGATAGAGCAGAGTGGACCACCCCATGAACC
TCGATTTAAATTCCAGGTTGTCATCAATGGCCGAGAGTTTCCCCCAGCTG
AAGCTGGAAGCAAGAAAGTGGCCAAGCAGGATGCAGCTATGAAAGCCATG
ACAATTCTGCTAGAGGAAGCCAAAGCCAAGGACAGTGGAAAATCAGAAGA
ATCATCCCACTATTCCACAGAGAAAGAATCAGAGAAGACTGCAGAGTCCC
AGACCCCCACCCCTTCAGCCACATCCTTCTTTTCTGGGAAGAGCCCCGTC
ACCACACTGCTTGAGTGTATGCACAAATTGGGGAACTCCTGCGAATTCCG
TCTCCTGTCCAAAGAAGGCCCTGCCCATGAACCCAAGTTCCAATACTGTG
TTGCAGTGGGAGCCCAAACTTTCCCCAGTGTGAGTGCTCCCAGCAAGAAA
GTGGCAAAGCAGATGGCCGCAGAGGAAGCCATGAAGGCCCTGCATGGGGA
GGCGACCAACTCCATGGCTTCTGATAACCAGCCTGAAGGTATGATCTCAG
AGTCACTTGATAACTTGGAATCCATGATGCCCAACAAGGTCAGGAAGATT
GGCGAGCTCGTGAGATACCTGAACACCAACCCTGTGGGTGGCCTTTTGGA
GTACGCCCGCTCCCATGGCTTTGCTGCTGAATTCAAGTTGGTCGACCAGT
CCGGACCTCCTCACGAGCCCAAGTTCGTTTACCAAGCAAAAGTTGGGGGT
CGCTGGTTCCCAGCCGTCTGCGCACACAGCAAGAAGCAAGGCAAGCAGGA
AGCAGCAGATGCGGCTCTCCGTGTCTTGATTGGGGAGAACGAGAAGGCAG
AACGCATGGGTTTCACAGAGGTAACCCCAGTGACAGGGGCCAGTCTCAGA
AGAACTATGCTCCTCCTCTCAAGGTCCCCAGAAGCACAGCCAAAGACACT
CCCTCTCACTGGCAGCACCTTCCATGACCAGATAGCCATGCTGAGCCACC
GGTGCTTCAACACTCTGACTAACAGCTTCCAGCCCTCCTTGCTCGGCCGC
AAGATTCTGGCCGCCATCATTATGAAAAAAGACTCTGAGGACATGGGTGT
CGTCGTCAGCTTGGGAACAGGGAATCGCTGTGTGAAAGGAGATTCTCTCA
GCCTAAAAGGAGAAACTGTCAATGACTGCCATGCAGAAATAATCTCCCGG
AGAGGCTTCATCAGGTTTCTCTACAGTGAGTTAATGAAATACAACTCCCA
GACTGCGAAGGATAGTATATTTGAACCTGCTAAGGGAGGAGAAAAGCTCC
AAATAAAAAAGACTGTGTCATTCCATCTGTATATCAGCACTGCTCCGTGT
GGAGATGGCGCCCTCTTTGACAAGTCCTGCAGCGACCGTGCTATGGAAAG
CACAGAATCCCGCCACTACCCTGTCTTCGAGAATCCCAAACAAGGAAAGC
TCCGCACCAAGGTGGAGAACGGAGAAGGCACAATCCCTGTGGAATCCAGT
GACATTGTGCCTACGTGGGATGGCATTCGGCTCGGGGAGAGACTCCGTAC
CATGTCCTGTAGTGACAAAATCCTACGCTGGAACGTGCTGGGCCTGCAAG
GGGCACTGTTGACCCACTTCCTGCAGCCCATTTATCTCAAATCTGTCACA
TTGGGTTACCTTTTCAGCCAAGGGCATCTGACCCGTGCTATTTGCTGTCG
TGTGACAAGAGATGGGAGTGCATTTGAGGATGGACTACGACATCCCTTTA
TTGTCAACCACCCCAAGGTTGGCAGAGTCAGCATATATGATTCCAAAAGG
CAATCCGGGAAGACTAAGGAGACAAGCGTCAACTGGTGTCTGGCTGATGG
CTATGACCTGGAGATCCTGGACGGTACCAGAGGCACTGTGGATGGGCCAC
GGAATGAATTGTCCCGGGTCTCCAAAAAGAACATTTTTCTTCTATTTAAG
AAGCTCTGCTCCTTCCGTTACCGCAGGGATCTACTGAGACTCTCCTATGG
TGAGGCCAAGAAAGCTGCCCGTGACTACGAGACGGCCAAGAACTACTTCA
AAAAAGGCCTGAAGGATATGGGCTATGGGAACTGGATTAGCAAACCCCAG
GAGGAAAAGAACTTTTATCTCTGCCCAGTATAGTATGCTCCAGTGACAGA
TGGATTAGGGTGTGTCATACTAGGGTGTGAGAGAGGTAGGTCGTAGCATT
CCTCATCACATGGTCAGGGGATTTTTTTTTCTCCTTTTTTTTTCTTTTTA
AGCCATAATTGGTGATACTGAAAACTTTGGGTTCCCATTTATCCTGCTTT
CTTTGGGATTGCTAGGCAAGGTCTGGCCAGGCCCCCCTTTTTTCCCCCAA
GTGAAGAGGCAGAAACCTAAGAAGTTATCTTTTCTTTCTACCCAAAGCAT
ACATAGTCACTGAGCACCTGCGGTCCATTTCCTCTTAAAAGTTTTGTTTT
GATTTGTTTCCATTTCCTTTCCCTTTGTGTTTGCTACACTGACCTCTTGC
GGTCTTGATTAGGTTTCAGTCAACTCTGGATCATGTCAGGGACTGATAAT
TTCATTTGTGGATTACGCAGACCCCTCTACTTCCCCTCTTTCCCTTCTGA
GATTCTTTCCTTGTGATCTGAATGTCTCCTTTTCCCCCTCAGAGGGCAAA
GAGGTGAACATAAAGGATTTGGTGAAACATTTGTAAGGGTAGGAGTTGAA
AACTGCAGTTCCCAGTGCCACGGAAGTGTGATTGGAGCCTGCAGATAATG
CCCAGCCATCCTCCCATCCTGCACTTTAGCCAGCTGCAGGGCGGGCAAGG
CAAGGAAAGCTGCTTCCCTGGAAGTGTATCACTTTCTCCGGCAGCTGGGA
AGTCTAGAACCAGCCAGACTGGGTTAAGGGAGCTGCTCAAGCAATAGCAG
AGGTTTCACCCGGCAGGATGACACAGACCACTTCCCAGGGAGCACGGGCA
TGCCTTGGAATATTGCCAAGCTTCCAGCTGCCTCTTCTCCTAAAGCATTC
CTAGGAATATTTTCCCCGCCAATGCTGGGCGTACACCCTAGCCAACGGGA
CAAATCCTAGAGGGTATAAAATCATCTCTGCTCAGATAATCATGACTTAG
CAAGAATAAGGGCAAAAAATCCTGTTGGCTTAACGTCACTGTTCCACCCG
GTGTAATATCTCTCATGACAGTGACACCAAGGGAAGTTGACTAAGTCACA
TGTAAATTAGGAGTGTTTTAAAGAATGCCATAGATGTTGATTCTTAACTG
CTACAGATAACCTGTAATTGAGCAGATTTAAAATTCAGGCATACTTTTCC
ATTTATCCAAGTGCTTTCATTTTTCCAGATGGCTTCAGAAGTAGGCTCGT
GGGCAGGGCGCAGACCTGATCTTTATAGGGTTGACATAGAAAGCAGTAGT
TGTGGGTGAAAGGGCAGGTTGTCTTCAAACTCTGTGAGGTAGAATCCTTT
GTCTATACCTCCATGAACATTGACTCGTGTGTTCAGAGCCTTTGGCCTCT
CTGTGGAGTCTGGCTCTCTGGCTCCTGTGCATTCTTTGAATAGTCACTCG
TAAAAACTGTCAGTGCTTGAAACTGTTTCCTTTACTCATGTTGAAGGGAC
TTTGTTGGCTTTTAGAGTGTTGGTCATGACTCCAAGAGCAGAGCAGGGAA
GAGCCCAAGCATAGACTTGGTGCCGTGGTGATGGCTGCAGTCCAGTTTTG
TGATGCTGCTTTTACGTGTCCCTCGATAACAGTCAGCTAGACACACTCAG
GAGGACTACTGAGGCTCTGCGACCTTCAGGAGCTGAGCCTGCCTCTCTCC
TTTAGATGACAGACCTTCATCTGGGAACGTGCTGAGCCAGCACCCTCAGA
TGATTTCCCTCCAAACTGCTGACTAGGTCATCCTCTGTCTGGTAGAGACA
TTCACATCTTTGCTTTTATTCTATGCTCTCTGTACTTTTGACCAAAAATT
GACCAAAGTAAGAAAATGCAAGTTCTAAAAATAGACTAAGGATGCCTTTG
CAGAACACCAAAGCATCCCAAGGAACTGGTAGGGAAGTGGCGCCTGTCTC
CTGGAGTGGAAGAGGCCTGCTCCCTGGCTCTGGGTCTGCTGGGGGCACAG
TAAATCAGTCTTGGCACCCACATCCAGGGCAGAGAGGTCTGTGGTTCTCA
GCATCAGAAGGCAGCGCAGCCCCTCTCCTCTTCAGGCTACAGGGTTGTCA
CCTGCTGAGTCCTCAGGTTGTTTGGCCTCTCTGGTCCATCTTGGGCATTA
GGTTCTCCAGCAGAGCTCTGGCCAGCTGCCTCTTCTTTAACTGGGAACAC
AGGCTCTCACAAGATCAGAACCCCCACTCACCCCCAAGATCTTATCTAGC
AAGCCTGTAGTATTCAGTTTCTGTTGTAGGAAGAGAGCGAGGCATCCCTG
AATTCCACGCATCTGCTGGAAACGAGCCGTGTCAGATCGCACATCCCTGC
GCCCCCATGCCCCTCTGAGTCACACAGGACAGAGGAGGCAGAGCTTCTGC
CCACTGTTATCTTCACTTTCTTTGTCCAGTCTTTTGTTTTTAATAAGCAG
TGACCCTCCCTACTCTTCTTTTTAATGATTTTTGTAGTTGATTTGTCTGA
ACTGTGGCTACTGTGCATTCCTTGAATAATCACTTGTAAAAATTGTCAGT
GCTTGAAGCTGTTTCCTTTACTCACATTGAAGGGACTTCGTTGGTTTTTT
GGAGTCTTGGTTGTGACTCCAAGAGCAGAGTGAGGAAGACCCCCAAGCAT
AGACTCGGGTACTGTGATGATGGCTGCAGTCCAGTTTTATGATTCTGCTT
TTATGTGTCCCTTGATAACAGTGACTTAACAATATACATTCCTCATAAAT
AAAAAAAAAACAAGAATCTGAATTCTTAGAAA

SEQ ID NO: 14 - Human ADAR1 - Exemplary p110 Transcript Variant Coding Sequence

ATGGCCGAGATCAAGGAGAAAATCTGCGACTATCTCTTCAATGTGTCTGA
CTCCTCTGCCCTGAATTTGGCTAAAAATATTGGCCTTACCAAGGCCCGAG
ATATAAATGCTGTGCTAATTGACATGGAAAGGCAGGGGGATGTCTATAGA
CAAGGGACAACCCCTCCCATATGGCATTTGACAGACAAGAAGCGAGAGAG
GATGCAAATCAAGAGAAATACGAACAGTGTTCCTGAAACCGCTCCAGCTG
CAATCCCTGAGACCAAAAGAAACGCAGAGTTCCTCACCTGTAATATACCC
ACATCAAATGCCTCAAATAACATGGTAACCACAGAAAAAGTGGAGAATGG
GCAGGAACCTGTCATAAAGTTAGAAAACAGGCAAGAGGCCAGACCAGAAC
CAGCAAGACTGAAACCACCTGTTCATTACAATGGCCCCTCAAAAGCAGGG
TATGTTGACTTTGAAAATGGCCAGTGGGCCACAGATGACATCCCAGATGA
CTTGAATAGTATCCGCGCAGCACCAGGTGAGTTTCGAGCCATCATGGAGA
TGCCCTCCTTCTACAGTCATGGCTTGCCACGGTGTTCACCCTACAAGAAA
CTGACAGAGTGCCAGCTGAAGAACCCCATCAGCGGGCTGTTAGAATATGC
CCAGTTCGCTAGTCAAACCTGTGAGTTCAACATGATAGAGCAGAGTGGAC
CACCCCATGAACCTCGATTTAAATTCCAGGTTGTCATCAATGGCCGAGAG
TTTCCCCCAGCTGAAGCTGGAAGCAAGAAAGTGGCCAAGCAGGATGCAGC
TATGAAAGCCATGACAATTCTGCTAGAGGAAGCCAAAGCCAAGGACAGTG
GAAAATCAGAAGAATCATCCCACTATTCCACAGAGAAAGAATCAGAGAAG
ACTGCAGAGTCCCAGACCCCCACCCCTTCAGCCACATCCTTCTTTTCTGG
GAAGAGCCCCGTCACCACACTGCTTGAGTGTATGCACAAATTGGGGAACT
CCTGCGAATTCCGTCTCCTGTCCAAAGAAGGCCCTGCCCATGAACCCAAG
TTCCAATACTGTGTTGCAGTGGGAGCCCAAACTTTCCCCAGTGTGAGTGC
TCCCAGCAAGAAAGTGGCAAAGCAGATGGCCGCAGAGGAAGCCATGAAGG
CCCTGCATGGGGAGGCGACCAACTCCATGGCTTCTGATAACCAGCCTGAA
GGTATGATCTCAGAGTCACTTGATAACTTGGAATCCATGATGCCCAACAA
GGTCAGGAAGATTGGCGAGCTCGTGAGATACCTGAACACCAACCCTGTGG
GTGGCCTTTTGGAGTACGCCCGCTCCCATGGCTTTGCTGCTGAATTCAAG
TTGGTCGACCAGTCCGGACCTCCTCACGAGCCCAAGTTCGTTTACCAAGC
AAAAGTTGGGGGTCGCTGGTTCCCAGCCGTCTGCGCACACAGCAAGAAGC
AAGGCAAGCAGGAAGCAGCAGATGCGGCTCTCCGTGTCTTGATTGGGGAG
AACGAGAAGGCAGAACGCATGGGTTTCACAGAGGTAACCCCAGTGACAGG
GGCCAGTCTCAGAAGAACTATGCTCCTCCTCTCAAGGTCCCCAGAAGCAC
AGCCAAAGACACTCCCTCTCACTGGCAGCACCTTCCATGACCAGATAGCC
ATGCTGAGCCACCGGTGCTTCAACACTCTGACTAACAGCTTCCAGCCCTC
CTTGCTCGGCCGCAAGATTCTGGCCGCCATCATTATGAAAAAAGACTCTG
AGGACATGGGTGTCGTCGTCAGCTTGGGAACAGGGAATCGCTGTGTAAAA
GGAGATTCTCTCAGCCTAAAAGGAGAAACTGTCAATGACTGCCATGCAGA
AATAATCTCCCGGAGAGGCTTCATCAGGTTTCTCTACAGTGAGTTAATGA
AATACAACTCCCAGACTGCGAAGGATAGTATATTTGAACCTGCTAAGGGA
GGAGAAAAGCTCCAAATAAAAAAGACTGTGTCATTCCATCTGTATATCAG
CACTGCTCCGTGTGGAGATGGCGCCCTCTTTGACAAGTCCTGCAGCGACC
GTGCTATGGAAAGCACAGAATCCCGCCACTACCCTGTCTTCGAGAATCCC
AAACAAGGAAAGCTCCGCACCAAGGTGGAGAACGGAGAAGGCACAATCCC
TGTGGAATCCAGTGACATTGTGCCTACGTGGGATGGCATTCGGCTCGGGG
AGAGACTCCGTACCATGTCCTGTAGTGACAAAATCCTACGCTGGAACGTG
CTGGGCCTGCAAGGGGCACTGTTGACCCACTTCCTGCAGCCCATTTATCT
CAAATCTGTCACATTGGGTTACCTTTTCAGCCAAGGGCATCTGACCCGTG
CTATTTGCTGTCGTGTGACAAGAGATGGGAGTGCATTTGAGGATGGACTA
CGACATCCCTTTATTGTCAACCACCCCAAGGTTGGCAGAGTCAGCATATA
TGATTCCAAAAGGCAATCCGGGAAGACTAAGGAGACAAGCGTCAACTGGT
GTCTGGCTGATGGCTATGACCTGGAGATCCTGGACGGTACCAGAGGCACT
GTGGATGGGCCACGGAATGAATTGTCCCGGGTCTCCAAAAAGAACATTTT
TCTTCTATTTAAGAAGCTCTGCTCCTTCCGTTACCGCAGGGATCTACTGA
GACTCTCCTATGGTGAGGCCAAGAAAGCTGCCCGTGACTACGAGACGGCC
AAGAACTACTTCAAAAAAGGCCTGAAGGATATGGGCTATGGGAACTGGAT
TAGCAAACCCCAGGAGGAAAAGAACTTTTATCTCTGCCCAGTATAG

SEQ ID NO: 15 - Human ADAR1 - Transcript Variant 4, 5, 7, 8, and 9 Coding Sequence

ATGGCCGAGATCAAGGAGAAAATCTGCGACTATCTCTTCAATGTGTCTGA
CTCCTCTGCCCTGAATTTGGCTAAAAATATTGGCCTTACCAAGGCCCGAG
ATATAAATGCTGTGCTAATTGACATGGAAAGGCAGGGGGATGTCTATAGA
CAAGGGACAACCCCTCCCATATGGCATTTGACAGACAAGAAGCGAGAGAG
GATGCAAATCAAGAGAAATACGAACAGTGTTCCTGAAACCGCTCCAGCTG
CAATCCCTGAGACCAAAAGAAACGCAGAGTTCCTCACCTGTAATATACCC
ACATCAAATGCCTCAAATAACATGGTAACCACAGAAAAAGTGGAGAATGG
GCAGGAACCTGTCATAAAGTTAGAAAACAGGCAAGAGGCCAGACCAGAAC
CAGCAAGACTGAAACCACCTGTTCATTACAATGGCCCCTCAAAAGCAGGG
TATGTTGACTTTGAAAATGGCCAGTGGGCCACAGATGACATCCCAGATGA
CTTGAATAGTATCCGCGCAGCACCAGGTGAGTTTCGAGCCATCATGGAGA
TGCCCTCCTTCTACAGTCATGGCTTGCCACGGTGTTCACCCTACAAGAAA
CTGACAGAGTGCCAGCTGAAGAACCCCATCAGCGGGCTGTTAGAATATGC
CCAGTTCGCTAGTCAAACCTGTGAGTTCAACATGATAGAGCAGAGTGGAC
CACCCCATGAACCTCGATTTAAATTCCAGGTTGTCATCAATGGCCGAGAG
TTTCCCCCAGCTGAAGCTGGAAGCAAGAAAGTGGCCAAGCAGGATGCAGC
TATGAAAGCCATGACAATTCTGCTAGAGGAAGCCAAAGCCAAGGACAGTG
GAAAATCAGAAGAATCATCCCACTATTCCACAGAGAAAGAATCAGAGAAG
ACTGCAGAGTCCCAGACCCCCACCCCTTCAGCCACATCCTTCTTTTCTGG
GAAGAGCCCCGTCACCACACTGCTTGAGTGTATGCACAAATTGGGGAACT
CCTGCGAATTCCGTCTCCTGTCCAAAGAAGGCCCTGCCCATGAACCCAAG
TTCCAATACTGTGTTGCAGTGGGAGCCCAAACTTTCCCCAGTGTGAGTGC
TCCCAGCAAGAAAGTGGCAAAGCAGATGGCCGCAGAGGAAGCCATGAAGG
CCCTGCATGGGGAGGCGACCAACTCCATGGCTTCTGATAACCAGCCTGAA
GGTATGATCTCAGAGTCACTTGATAACTTGGAATCCATGATGCCCAACAA
GGTCAGGAAGATTGGCGAGCTCGTGAGATACCTGAACACCAACCCTGTGG
GTGGCCTTTTGGAGTACGCCCGCTCCCATGGCTTTGCTGCTGAATTCAAG
TTGGTCGACCAGTCCGGACCTCCTCACGAGCCCAAGTTCGTTTACCAAGC
AAAAGTTGGGGGTCGCTGGTTCCCAGCCGTCTGCGCACACAGCAAGAAGC
AAGGCAAGCAGGAAGCAGCAGATGCGGCTCTCCGTGTCTTGATTGGGGAG
AACGAGAAGGCAGAACGCATGGGTTTCACAGAGGTAACCCCAGTGACAGG
GGCCAGTCTCAGAAGAACTATGCTCCTCCTCTCAAGGTCCCCAGAAGCAC
AGCCAAAGACACTCCCTCTCACTGGCAGCACCTTCCATGACCAGATAGCC
ATGCTGAGCCACCGGTGCTTCAACACTCTGACTAACAGCTTCCAGCCCTC
CTTGCTCGGCCGCAAGATTCTGGCCGCCATCATTATGAAAAAAGACTCTG
AGGACATGGGTGTCGTCGTCAGCTTGGGAACAGGGAATCGCTGTGTGAAA
GGAGATTCTCTCAGCCTAAAAGGAGAAACTGTCAATGACTGCCATGCAGA
AATAATCTCCCGGAGAGGCTTCATCAGGTTTCTCTACAGTGAGTTAATGA
AATACAACTCCCAGACTGCGAAGGATAGTATATTTGAACCTGCTAAGGGA
GGAGAAAAGCTCCAAATAAAAAAGACTGTGTCATTCCATCTGTATATCAG
CACTGCTCCGTGTGGAGATGGCGCCCTCTTTGACAAGTCCTGCAGCGACC
GTGCTATGGAAAGCACAGAATCCCGCCACTACCCTGTCTTCGAGAATCCC
AAACAAGGAAAGCTCCGCACCAAGGTGGAGAACGGAGAAGGCACAATCCC
TGTGGAATCCAGTGACATTGTGCCTACGTGGGATGGCATTCGGCTCGGGG
AGAGACTCCGTACCATGTCCTGTAGTGACAAAATCCTACGCTGGAACGTG
CTGGGCCTGCAAGGGGCACTGTTGACCCACTTCCTGCAGCCCATTTATCT
CAAATCTGTCACATTGGGTTACCTTTTCAGCCAAGGGCATCTGACCCGTG
CTATTTGCTGTCGTGTGACAAGAGATGGGAGTGCATTTGAGGATGGACTA
CGACATCCCTTTATTGTCAACCACCCCAAGGTTGGCAGAGTCAGCATATA
TGATTCCAAAAGGCAATCCGGGAAGACTAAGGAGACAAGCGTCAACTGGT
GTCTGGCTGATGGCTATGACCTGGAGATCCTGGACGGTACCAGAGGCACT
GTGGATGGGCCACGGAATGAATTGTCCCGGGTCTCCAAAAAGAACATTTT
TCTTCTATTTAAGAAGCTCTGCTCCTTCCGTTACCGCAGGGATCTACTGA
GACTCTCCTATGGTGAGGCCAAGAAAGCTGCCCGTGACTACGAGACGGCC
AAGAACTACTTCAAAAAAGGCCTGAAGGATATGGGCTATGGGAACTGGAT
TAGCAAACCCCAGGAGGAAAAGAACTTTTATCTCTGCCCAGTATAG

SEQ ID NO: 16 - Human ADAR1 - Transcript Variant 4, 5, 7, 8, and 9 Amino Acid Sequence (also known as isoform-d, and/or p110)

MAEIKEKICDYLFNVSDSSALNLAKNIGLTKARDINAVLIDMERQGDVYR
QGTTPPIWHLTDKKRERMQIKRNTNSVPETAPAAIPETKRNAEFLTCNIP
TSNASNNMVTTEKVENGQEPVIKLENRQEARPEPARLKPPVHYNGPSKAG
YVDFENGQWATDDIPDDLNSIRAAPGEFRAIMEMPSFYSHGLPRCSPYKK
LTECQLKNPISGLLEYAQFASQTCEFNMIEQSGPPHEPRFKFQVVINGRE
FPPAEAGSKKVAKQDAAMKAMTILLEEAKAKDSGKSEESSHYSTEKESEK
TAESQTPTPSATSFFSGKSPVTTLLECMHKLGNSCEFRLLSKEGPAHEPK
FQYCVAVGAQTFPSVSAPSKKVAKQMAAEEAMKALHGEATNSMASDNQPE
GMISESLDNLESMMPNKVRKIGELVRYLNTNPVGGLLEYARSHGFAAEFK
LVDQSGPPHEPKFVYQAKVGGRWFPAVCAHSKKQGKQEAADAALRVLIGE
NEKAERMGFTEVTPVTGASLRRTMLLLSRSPEAQPKTLPLTGSTFHDQIA
MLSHRCFNTLTNSFQPSLLGRKILAAIIMKKDSEDMGVVVSLGTGNRCVK
GDSLSLKGETVNDCHAEIISRRGFIRFLYSELMKYNSQTAKDSIFEPAKG
GEKLQIKKTVSFHLYISTAPCGDGALFDKSCSDRAMESTESRHYPVFENP
KQGKLRTKVENGEGTIPVESSDIVPTWDGIRLGERLRTMSCSDKILRWNV
LGLQGALLTHFLQPIYLKSVTLGYLFSQGHLTRAICCRVTRDGSAFEDGL
RHPFIVNHPKVGRVSIYDSKRQSGKTKETSVNWCLADGYDLEILDGTRGT
VDGPRNELSRVSKKNIFLLFKKLCSFRYRRDLLRLSYGEAKKAARDYETA
KNYFKKGLKDMGYGNWISKPQEEKNFYLCPV

In certain embodiments, an ADAR1 polynucleotide or a human ADAR1 gene incorporated into a non-human animal (e.g., a rodent, e.g., a rat or mouse) is represented by or comprises a sequence encoding a human ADAR1 transcript variant 51 or a characteristic portion thereof. In certain embodiments, an ADAR1 polynucleotide or a human ADAR1 gene comprises, or consists of, a nucleotide sequence that is the same as, differs by no more than 1, 2, 3, 4, 5, 6, 7, 8, 9, or 10 nucleotides from, or has at least has at least 90%, 92%, 94%, 96%, 98%, or 99% homology with, the nucleotide sequence of SEQ ID NO: 17 or a characteristic portion thereof. In some embodiments, an ADAR1 polynucleotide or a human ADAR1 gene comprises, or consists of, a nucleotide sequence that is the same as the nucleotide sequence of SEQ ID NO: 17 or a characteristic portion thereof. In certain embodiments, an encoded human ADAR1 Amino Acid sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the Amino Acid sequence of SEQ ID NO: 16 or a characteristic portion thereof. In certain embodiments, the amino acid sequence of an encoded and/or expressed ADAR1 polypeptide or a characteristic portion thereof, is or comprises a sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the amino acid sequence of SEQ ID NO: 16 or a characteristic portion thereof.

SEQ ID NO: 17 - Human ADAR1 - Transcript Variant 5 cDNA Sequence

CCTGGCCAGCATGGTGAAACCCCATCTCTACTAAAAATACAAAAATTAGC
TGGGTGTGGTGGCGTGCGCCTGTAATCCCAGCTACTCCGGAGGCTGAGGC
AGGAGAATCACTTGAACCCGGGAGGCGGAGATTGCAGTGAGCTGAGATCA
CACTGCACTCCAGCCTGATTGCAGTGAGCCGAGATCATGCCACTGCACTC
CAGCTTGGCAACAGAGCGAGACTCCGTCTCACAAGAAAAAAAATAACCGG
GTATTCCCTCAGCGGATACTACACCCATCCATTTCAAGGCTATGAGCACA
GACAGCTCAGGTACCAGCAGCCTGGGCCAGGATCTTCCCCCAGTAGTTTC
CTGCTTAAGCAAATAGAATTTCTCAAGGGGCAGCTCCCAGAAGCACCGGT
GATTGGAAAGCAGACACCGTCACTGCCACCTTCCCTCCCAGGACTCCGGC
CAAGGTTTCCAGTACTACTTGCCTCCAGTACCAGAGGCAGGCAAGTGGAC
ATCAGGGGTGTCCCCAGGGGCGTGCATCTCAGAAGTCAGGGGCTCCAGAG
AGGGTTCCAGCATCCTTCACCACGTGGCAGGAGTCTGCCACAGAGAGGTG
TTGATTGCCTTTCCTCACATTTCCAGGAACTGAGTATCTACCAAGATCAG
GAACAAAGGATCTTAAAGTTCCTGGAAGAGCTTGGGGAAGGGAAGGCCAC
CACAGCACATGATCTGTCTGGGAAACTTGGGACTCCGAAGAAAGAAATCA
ATCGAGTTTTATACTCCCTGGCAAAGAAGGGCAAGCTACAGAAAGAGGCA
GGAACACCCCCTTTGTGGAAAATCGCGGTCTCCACTCAGGCTTGGAACCA
GCACAGCGGAGTGGTAAGACCAGACGGTCATAGCCAAGGAGCCCCAAACT
CAGACCCGAGTTTGGAACCGGAAGACAGAAACTCCACATCTGTCTCAGAA
GATCTTCTTGAGCCTTTTATTGCAGTCTCAGCTCAGGCTTGGAACCAGCA
CAGCGGAGTGGTAAGACCAGACAGTCATAGCCAAGGATCCCCAAACTCAG
ACCCAGGTTTGGAACCTGAAGACAGCAACTCCACATCTGCCTTGGAAGAT
CCTCTTGAGTTTTTAGACATGGCCGAGATCAAGGAGAAAATCTGCGACTA
TCTCTTCAATGTGTCTGACTCCTCTGCCCTGAATTTGGCTAAAAATATTG
GCCTTACCAAGGCCCGAGATATAAATGCTGTGCTAATTGACATGGAAAGG
CAGGGGGATGTCTATAGACAAGGGACAACCCCTCCCATATGGCATTTGAC
AGACAAGAAGCGAGAGAGGATGCAAATCAAGAGAAATACGAACAGTGTTC
CTGAAACCGCTCCAGCTGCAATCCCTGAGACCAAAAGAAACGCAGAGTTC
CTCACCTGTAATATACCCACATCAAATGCCTCAAATAACATGGTAACCAC
AGAAAAAGTGGAGAATGGGCAGGAACCTGTCATAAAGTTAGAAAACAGGC
AAGAGGCCAGACCAGAACCAGCAAGACTGAAACCACCTGTTCATTACAAT
GGCCCCTCAAAAGCAGGGTATGTTGACTTTGAAAATGGCCAGTGGGCCAC
AGATGACATCCCAGATGACTTGAATAGTATCCGCGCAGCACCAGGTGAGT
TTCGAGCCATCATGGAGATGCCCTCCTTCTACAGTCATGGCTTGCCACGG
TGTTCACCCTACAAGAAACTGACAGAGTGCCAGCTGAAGAACCCCATCAG
CGGGCTGTTAGAATATGCCCAGTTCGCTAGTCAAACCTGTGAGTTCAACA
TGATAGAGCAGAGTGGACCACCCCATGAACCTCGATTTAAATTCCAGGTT
GTCATCAATGGCCGAGAGTTTCCCCCAGCTGAAGCTGGAAGCAAGAAAGT
GGCCAAGCAGGATGCAGCTATGAAAGCCATGACAATTCTGCTAGAGGAAG
CCAAAGCCAAGGACAGTGGAAAATCAGAAGAATCATCCCACTATTCCACA
GAGAAAGAATCAGAGAAGACTGCAGAGTCCCAGACCCCCACCCCTTCAGC
CACATCCTTCTTTTCTGGGAAGAGCCCCGTCACCACACTGCTTGAGTGTA
TGCACAAATTGGGGAACTCCTGCGAATTCCGTCTCCTGTCCAAAGAAGGC
CCTGCCCATGAACCCAAGTTCCAATACTGTGTTGCAGTGGGAGCCCAAAC
TTTCCCCAGTGTGAGTGCTCCCAGCAAGAAAGTGGCAAAGCAGATGGCCG
CAGAGGAAGCCATGAAGGCCCTGCATGGGGAGGCGACCAACTCCATGGCT
TCTGATAACCAGCCTGAAGGTATGATCTCAGAGTCACTTGATAACTTGGA
ATCCATGATGCCCAACAAGGTCAGGAAGATTGGCGAGCTCGTGAGATACC
TGAACACCAACCCTGTGGGTGGCCTTTTGGAGTACGCCCGCTCCCATGGC
TTTGCTGCTGAATTCAAGTTGGTCGACCAGTCCGGACCTCCTCACGAGCC
CAAGTTCGTTTACCAAGCAAAAGTTGGGGGTCGCTGGTTCCCAGCCGTCT
GCGCACACAGCAAGAAGCAAGGCAAGCAGGAAGCAGCAGATGCGGCTCTC
CGTGTCTTGATTGGGGAGAACGAGAAGGCAGAACGCATGGGTTTCACAGA
GGTAACCCCAGTGACAGGGGCCAGTCTCAGAAGAACTATGCTCCTCCTCT
CAAGGTCCCCAGAAGCACAGCCAAAGACACTCCCTCTCACTGGCAGCACC
TTCCATGACCAGATAGCCATGCTGAGCCACCGGTGCTTCAACACTCTGAC
TAACAGCTTCCAGCCCTCCTTGCTCGGCCGCAAGATTCTGGCCGCCATCA
TTATGAAAAAAGACTCTGAGGACATGGGTGTCGTCGTCAGCTTGGGAACA
GGGAATCGCTGTGTGAAAGGAGATTCTCTCAGCCTAAAAGGAGAAACTGT
CAATGACTGCCATGCAGAAATAATCTCCCGGAGAGGCTTCATCAGGTTTC
TCTACAGTGAGTTAATGAAATACAACTCCCAGACTGCGAAGGATAGTATA
TTTGAACCTGCTAAGGGAGGAGAAAAGCTCCAAATAAAAAAGACTGTGTC
ATTCCATCTGTATATCAGCACTGCTCCGTGTGGAGATGGCGCCCTCTTTG
ACAAGTCCTGCAGCGACCGTGCTATGGAAAGCACAGAATCCCGCCACTAC
CCTGTCTTCGAGAATCCCAAACAAGGAAAGCTCCGCACCAAGGTGGAGAA
CGGAGAAGGCACAATCCCTGTGGAATCCAGTGACATTGTGCCTACGTGGG
ATGGCATTCGGCTCGGGGAGAGACTCCGTACCATGTCCTGTAGTGACAAA
ATCCTACGCTGGAACGTGCTGGGCCTGCAAGGGGCACTGTTGACCCACTT
CCTGCAGCCCATTTATCTCAAATCTGTCACATTGGGTTACCTTTTCAGCC
AAGGGCATCTGACCCGTGCTATTTGCTGTCGTGTGACAAGAGATGGGAGT
GCATTTGAGGATGGACTACGACATCCCTTTATTGTCAACCACCCCAAGGT
TGGCAGAGTCAGCATATATGATTCCAAAAGGCAATCCGGGAAGACTAAGG
AGACAAGCGTCAACTGGTGTCTGGCTGATGGCTATGACCTGGAGATCCTG
GACGGTACCAGAGGCACTGTGGATGGGCCACGGAATGAATTGTCCCGGGT
CTCCAAAAAGAACATTTTTCTTCTATTTAAGAAGCTCTGCTCCTTCCGTT
ACCGCAGGGATCTACTGAGACTCTCCTATGGTGAGGCCAAGAAAGCTGCC
CGTGACTACGAGACGGCCAAGAACTACTTCAAAAAAGGCCTGAAGGATAT
GGGCTATGGGAACTGGATTAGCAAACCCCAGGAGGAAAAGAACTTTTATC
TCTGCCCAGTATAGTATGCTCCAGTGACAGATGGATTAGGGTGTGTCATA
CTAGGGTGTGAGAGAGGTAGGTCGTAGCATTCCTCATCACATGGTCAGGG
GATTTTTTTTTCTCCTTTTTTTTTCTTTTTAAGCCATAATTGGTGATACT
GAAAACTTTGGGTTCCCATTTATCCTGCTTTCTTTGGGATTGCTAGGCAA
GGTCTGGCCAGGCCCCCCTTTTTTCCCCCAAGTGAAGAGGCAGAAACCTA
AGAAGTTATCTTTTCTTTCTACCCAAAGCATACATAGTCACTGAGCACCT
GCGGTCCATTTCCTCTTAAAAGTTTTGTTTTGATTTGTTTCCATTTCCTT
TCCCTTTGTGTTTGCTACACTGACCTCTTGCGGTCTTGATTAGGTTTCAG
TCAACTCTGGATCATGTCAGGGACTGATAATTTCATTTGTGGATTACGCA
GACCCCTCTACTTCCCCTCTTTCCCTTCTGAGATTCTTTCCTTGTGATCT
GAATGTCTCCTTTTCCCCCTCAGAGGGCAAAGAGGTGAACATAAAGGATT
TGGTGAAACATTTGTAAGGGTAGGAGTTGAAAACTGCAGTTCCCAGTGCC
ACGGAAGTGTGATTGGAGCCTGCAGATAATGCCCAGCCATCCTCCCATCC
TGCACTTTAGCCAGCTGCAGGGCGGGCAAGGCAAGGAAAGCTGCTTCCCT
GGAAGTGTATCACTTTCTCCGGCAGCTGGGAAGTCTAGAACCAGCCAGAC
TGGGTTAAGGGAGCTGCTCAAGCAATAGCAGAGGTTTCACCCGGCAGGAT
GACACAGACCACTTCCCAGGGAGCACGGGCATGCCTTGGAATATTGCCAA
GCTTCCAGCTGCCTCTTCTCCTAAAGCATTCCTAGGAATATTTTCCCCGC
CAATGCTGGGCGTACACCCTAGCCAACGGGACAAATCCTAGAGGGTATAA
AATCATCTCTGCTCAGATAATCATGACTTAGCAAGAATAAGGGCAAAAAA
TCCTGTTGGCTTAACGTCACTGTTCCACCCGGTGTAATATCTCTCATGAC
AGTGACACCAAGGGAAGTTGACTAAGTCACATGTAAATTAGGAGTGTTTT
AAAGAATGCCATAGATGTTGATTCTTAACTGCTACAGATAACCTGTAATT
GAGCAGATTTAAAATTCAGGCATACTTTTCCATTTATCCAAGTGCTTTCA
TTTTTCCAGATGGCTTCAGAAGTAGGCTCGTGGGCAGGGCGCAGACCTGA
TCTTTATAGGGTTGACATAGAAAGCAGTAGTTGTGGGTGAAAGGGCAGGT
TGTCTTCAAACTCTGTGAGGTAGAATCCTTTGTCTATACCTCCATGAACA
TTGACTCGTGTGTTCAGAGCCTTTGGCCTCTCTGTGGAGTCTGGCTCTCT
GGCTCCTGTGCATTCTTTGAATAGTCACTCGTAAAAACTGTCAGTGCTTG
AAACTGTTTCCTTTACTCATGTTGAAGGGACTTTGTTGGCTTTTAGAGTG
TTGGTCATGACTCCAAGAGCAGAGCAGGGAAGAGCCCAAGCATAGACTTG
GTGCCGTGGTGATGGCTGCAGTCCAGTTTTGTGATGCTGCTTTTACGTGT
CCCTCGATAACAGTCAGCTAGACACACTCAGGAGGACTACTGAGGCTCTG
CGACCTTCAGGAGCTGAGCCTGCCTCTCTCCTTTAGATGACAGACCTTCA
TCTGGGAACGTGCTGAGCCAGCACCCTCAGATGATTTCCCTCCAAACTGC
TGACTAGGTCATCCTCTGTCTGGTAGAGACATTCACATCTTTGCTTTTAT
TCTATGCTCTCTGTACTTTTGACCAAAAATTGACCAAAGTAAGAAAATGC
AAGTTCTAAAAATAGACTAAGGATGCCTTTGCAGAACACCAAAGCATCCC
AAGGAACTGGTAGGGAAGTGGCGCCTGTCTCCTGGAGTGGAAGAGGCCTG
CTCCCTGGCTCTGGGTCTGCTGGGGGCACAGTAAATCAGTCTTGGCACCC
ACATCCAGGGCAGAGAGGTCTGTGGTTCTCAGCATCAGAAGGCAGCGCAG
CCCCTCTCCTCTTCAGGCTACAGGGTTGTCACCTGCTGAGTCCTCAGGTT
GTTTGGCCTCTCTGGTCCATCTTGGGCATTAGGTTCTCCAGCAGAGCTCT
GGCCAGCTGCCTCTTCTTTAACTGGGAACACAGGCTCTCACAAGATCAGA
ACCCCCACTCACCCCCAAGATCTTATCTAGCAAGCCTGTAGTATTCAGTT
TCTGTTGTAGGAAGAGAGCGAGGCATCCCTGAATTCCACGCATCTGCTGG
AAACGAGCCGTGTCAGATCGCACATCCCTGCGCCCCCATGCCCCTCTGAG
TCACACAGGACAGAGGAGGCAGAGCTTCTGCCCACTGTTATCTTCACTTT
CTTTGTCCAGTCTTTTGTTTTTAATAAGCAGTGACCCTCCCTACTCTTCT
TTTTAATGATTTTTGTAGTTGATTTGTCTGAACTGTGGCTACTGTGCATT
CCTTGAATAATCACTTGTAAAAATTGTCAGTGCTTGAAGCTGTTTCCTTT
ACTCACATTGAAGGGACTTCGTTGGTTTTTTGGAGTCTTGGTTGTGACTC
CAAGAGCAGAGTGAGGAAGACCCCCAAGCATAGACTCGGGTACTGTGATG
ATGGCTGCAGTCCAGTTTTATGATTCTGCTTTTATGTGTCCCTTGATAAC
AGTGACTTAACAATATACATTCCTCATAAATAAAAAAAAAACAAGAATCT
GAATTCTTAGAAA

In certain embodiments, an ADAR1 polynucleotide or a human ADAR1 gene incorporated into a non-human animal (e.g., a rodent, e.g., a rat or mouse) is represented by or comprises a sequence encoding a human ADAR1 transcript variant 6 or a characteristic portion thereof. In certain embodiments, an ADAR1 polynucleotide or a human ADAR1 gene comprises, or consists of, a nucleotide sequence that is the same as, differs by no more than 1, 2, 3, 4, 5, 6, 7, 8, 9, or 10 nucleotides from, or has at least has at least 90%, 92%, 94%, 96%, 98%, or 99% homology with, the nucleotide sequence of SEQ ID NO: 18, or SEQ ID NO: 19 or a characteristic portion thereof. In some embodiments, an ADAR1 polynucleotide or a human ADAR1 gene comprises, or consists of, a nucleotide sequence that is the same as the nucleotide sequence of SEQ ID NO: 18, or SEQ ID NO: 19 or a characteristic portion thereof. In certain embodiments, an encoded human ADAR1 Amino Acid sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the Amino Acid sequence of SEQ ID NO: 20 or a characteristic portion thereof. In certain embodiments, the amino acid sequence of an encoded and/or expressed ADAR1 polypeptide or a characteristic portion thereof, is or comprises a sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the amino acid sequence of SEQ ID NO: 20 or a characteristic portion thereof.

SEQ ID NO: 18 - Human ADAR1 - Transcript Variant 6 cDNA Sequence

TGATGTCACCAATCTGCGACCAGACCATTGATTCCCGACTGAAGGGGTAT
TCCCTCAGCGGATACTACACCCATCCATTTCAAGGCTATGAGCACAGACA
GCTCAGGTACCAGCAGCCTGGGCCAGGATCTTCCCCCAGTAGTTTCCTGC
TTAAGCAAATAGAATTTCTCAAGGGGCAGCTCCCAGAAGCACCGGTGATT
GGAAAGCAGACACCGTCACTGCCACCTTCCCTCCCAGGACTCCGGCCAAG
GTTTCCAGTACTACTTGCCTCCAGTACCAGAGGCAGGCAAGTGGACATCA
GGGGTGTCCCCAGGGGCGTGCATCTCAGAAGTCAGGGGCTCCAGAGAGGG
TTCCAGCATCCTTCACCACGTGGCAGGAGTCTGCCACAGAGAGGTGTTGA
TTGCCTTTCCTCACATTTCCAGGAACTGAGTATCTACCAAGATCAGGAAC
AAAGGATCTTAAAGTTCCTGGAAGAGCTTGGGGAAGGGAAGGCCACCACA
GCACATGATCTGTCTGGGAAACTTGGGACTCCGAAGAAAGAAATCAATCG
AGTTTTATACTCCCTGGCAAAGAAGGGCAAGCTACAGAAAGAGGCAGGAA
CACCCCCTTTGTGGAAAATCGCGGTCTCCACTCAGGCTTGGAACCAGCAC
AGCGGAGTGGTAAGACCAGACGGTCATAGCCAAGGAGCCCCAAACTCAGA
CCCGAGTTTGGAACCGGAAGACAGAAACTCCACATCTGTCTCAGAAGATC
TTCTTGAGCCTTTTATTGCAGTCTCAGCTCAGGCTTGGAACCAGCACAGC
GGAGTGGTAAGACCAGACAGTCATAGCCAAGGATCCCCAAACTCAGACCC
AGGTTTGGAACCTGAAGACAGCAACTCCACATCTGCCTTGGAAGATCCTC
TTGAGTTTTTAGACATGGCCGAGATCAAGGAGAAAATCTGCGACTATCTC
TTCAATGTGTCTGACTCCTCTGCCCTGAATTTGGCTAAAAATATTGGCCT
TACCAAGGCCCGAGATATAAATGCTGTGCTAATTGACATGGAAAGGCAGG
GGGATGTCTATAGACAAGGGACAACCCCTCCCATATGGCATTTGACAGAC
AAGAAGCGAGAGAGGATGCAAATCAAGAGAAATACGAACAGTGTTCCTGA
AACCGCTCCAGCTGCAATCCCTGAGACCAAAAGAAACGCAGAGTTCCTCA
CCTGTAATATACCCACATCAAATGCCTCAAATAACATGGTAACCACAGAA
AAAGTGGAGAATGGGCAGGAACCTGTCATAAAGTTAGAAAACAGGCAAGA
GGCCAGACCAGAACCAGCAAGACTGAAACCACCTGTTCATTACAATGGCC
CCTCAAAAGCAGGGTATGTTGACTTTGAAAATGGCCAGTGGGCCACAGAT
GACATCCCAGATGACTTGAATAGTATCCGCGCAGCACCAGGTGAGTTTCG
AGCCATCATGGAGATGCCCTCCTTCTACAGTCATGGCTTGCCACGGTGTT
CACCCTACAAGAAACTGACAGAGTGCCAGCTGAAGAACCCCATCAGCGGG
CTGTTAGAATATGCCCAGTTCGCTAGTCAAACCTGTGAGTTCAACATGAT
AGAGCAGAGTGGACCACCCCATGAACCTCGATTTAAATTCCAGGTTGTCA
TCAATGGCCGAGAGTTTCCCCCAGCTGAAGCTGGAAGCAAGAAAGTGGCC
AAGCAGGATGCAGCTATGAAAGCCATGACAATTCTGCTAGAGGAAGCCAA
AGCCAAGGACAGTGGAAAATCAGAAGAATCATCCCACTATTCCACAGAGA
AAGAATCAGAGAAGACTGCAGAGTCCCAGACCCCCACCCCTTCAGCCACA
TCCTTCTTTTCTGGGAAGAGCCCCGTCACCACACTGCTTGAGTGTATGCA
CAAATTGGGGAACTCCTGCGAATTCCGTCTCCTGTCCAAAGAAGGCCCTG
CCCATGAACCCAAGTTCCAATACTGTGTTGCAGTGGGAGCCCAAACTTTC
CCCAGTGTGAGTGCTCCCAGCAAGAAAGTGGCAAAGCAGATGGCCGCAGA
GGAAGCCATGAAGGCCCTGCATGGGGAGGCGACCAACTCCATGGCTTCTG
ATAACCAGCCTGAAGGTATGATCTCAGAGTCACTTGATAACTTGGAATCC
ATGATGCCCAACAAGGTCAGGAAGATTGGCGAGCTCGTGAGATACCTGAA
CACCAACCCTGTGGGTGGCCTTTTGGAGTACGCCCGCTCCCATGGCTTTG
CTGCTGAATTCAAGTTGGTCGACCAGTCCGGACCTCCTCACGAGCCCAAG
TTCGTTTACCAAGCAAAAGTTGGGGGTCGCTGGTTCCCAGCCGTCTGCGC
ACACAGCAAGAAGCAAGGCAAGCAGGAAGCAGCAGATGCGGCTCTCCGTG
TCTTGATTGGGGAGAACGAGAAGGCAGAACGCATGGGTTTCACAGAGGTA
ACCCCAGTGACAGGGGCCAGTCTCAGAAGAACTATGCTCCTCCTCTCAAG
GTCCCCAGAAGCACAGCCAAAGACACTCCCTCTCACTGGCAGCACCTTCC
ATGACCAGATAGCCATGCTGAGCCACCGGTGCTTCAACACTCTGACTAAC
AGCTTCCAGCCCTCCTTGCTCGGCCGCAAGATTCTGGCCGCCATCATTAT
GAAAAAAGACTCTGAGGACATGGGTGTCGTCGTCAGCTTGGGAACAGGGA
ATCGCTGTGTGAAAGGAGATTCTCTCAGCCTAAAAGGAGAAACTGTCAAT
GACTGCCATGCAGAAATAATCTCCCGGAGAGGCTTCATCAGGTTTCTCTA
CAGTGAGTTAATGAAATACAACTCCCAGACTGCGAAGGATAGTATATTTG
AACCTGCTAAGGGAGGAGAAAAGCTCCAAATAAAAAAGACTGTGTCATTC
CATCTGTATATCAGCACTGCTCCGTGTGGAGATGGCGCCCTCTTTGACAA
GTCCTGCAGCGACCGTGCTATGGAAAGCACAGAATCCCGCCACTACCCTG
TCTTCGAGAATCCCAAACAAGGAAAGCTCCGCACCAAGGTGGAGAACGGA
GAAGGCACAATCCCTGTGGAATCCAGTGACATTGTGCCTACGTGGGATGG
CATTCGGCTCGGGGAGAGACTCCGTACCATGTCCTGTAGTGACAAAATCC
TACGCTGGAACGTGCTGGGCCTGCAAGGGGCACTGTTGACCCACTTCCTG
CAGCCCATTTATCTCAAATCTGTCACATTGGGTTACCTTTTCAGCCAAGG
GCATCTGACCCGTGCTATTTGCTGTCGTGTGACAAGAGATGGGAGTGCAT
TTGAGGATGGACTACGACATCCCTTTATTGTCAACCACCCCAAGGTTGGC
AGAGTCAGCATATATGATTCCAAAAGGCAATCCGGGAAGACTAAGGAGAC
AAGCGTCAACTGGTGTCTGGCTGATGGCTATGACCTGGAGATCCTGGACG
GTACCAGAGGCACTGTGGATGGGCCACGGAATGAATTGTCCCGGGTCTCC
AAAAAGAACATTTTTCTTCTATTTAAGAAGCTCTGCTCCTTCCGTTACCG
CAGGGATCTACTGAGACTCTCCTATGGTGAGGCCAAGAAAGCTGCCCGTG
ACTACGAGACGGCCAAGAACTACTTCAAAAAAGGCCTGAAGGATATGGGC
TATGGGAACTGGATTAGCAAACCCCAGGAGGAAAAGAACTTTTATCTCTG
CCCAGTATAGTATGCTCCAGTGACAGATGGATTAGGGTGTGTCATACTAG
GGTGTGAGAGAGGTAGGTCGTAGCATTCCTCATCACATGGTCAGGGGATT
TTTTTTTCTCCTTTTTTTTTCTTTTTAAGCCATAATTGGTGATACTGAAA
ACTTTGGGTTCCCATTTATCCTGCTTTCTTTGGGATTGCTAGGCAAGGTC
TGGCCAGGCCCCCCTTTTTTCCCCCAAGTGAAGAGGCAGAAACCTAAGAA
GTTATCTTTTCTTTCTACCCAAAGCATACATAGTCACTGAGCACCTGCGG
TCCATTTCCTCTTAAAAGTTTTGTTTTGATTTGTTTCCATTTCCTTTCCC
TTTGTGTTTGCTACACTGACCTCTTGCGGTCTTGATTAGGTTTCAGTCAA
CTCTGGATCATGTCAGGGACTGATAATTTCATTTGTGGATTACGCAGACC
CCTCTACTTCCCCTCTTTCCCTTCTGAGATTCTTTCCTTGTGATCTGAAT
GTCTCCTTTTCCCCCTCAGAGGGCAAAGAGGTGAACATAAAGGATTTGGT
GAAACATTTGTAAGGGTAGGAGTTGAAAACTGCAGTTCCCAGTGCCACGG
AAGTGTGATTGGAGCCTGCAGATAATGCCCAGCCATCCTCCCATCCTGCA
CTTTAGCCAGCTGCAGGGCGGGCAAGGCAAGGAAAGCTGCTTCCCTGGAA
GTGTATCACTTTCTCCGGCAGCTGGGAAGTCTAGAACCAGCCAGACTGGG
TTAAGGGAGCTGCTCAAGCAATAGCAGAGGTTTCACCCGGCAGGATGACA
CAGACCACTTCCCAGGGAGCACGGGCATGCCTTGGAATATTGCCAAGCTT
CCAGCTGCCTCTTCTCCTAAAGCATTCCTAGGAATATTTTCCCCGCCAAT
GCTGGGCGTACACCCTAGCCAACGGGACAAATCCTAGAGGGTATAAAATC
ATCTCTGCTCAGATAATCATGACTTAGCAAGAATAAGGGCAAAAAATCCT
GTTGGCTTAACGTCACTGTTCCACCCGGTGTAATATCTCTCATGACAGTG
ACACCAAGGGAAGTTGACTAAGTCACATGTAAATTAGGAGTGTTTTAAAG
AATGCCATAGATGTTGATTCTTAACTGCTACAGATAACCTGTAATTGAGC
AGATTTAAAATTCAGGCATACTTTTCCATTTATCCAAGTGCTTTCATTTT
TCCAGATGGCTTCAGAAGTAGGCTCGTGGGCAGGGCGCAGACCTGATCTT
TATAGGGTTGACATAGAAAGCAGTAGTTGTGGGTGAAAGGGCAGGTTGTC
TTCAAACTCTGTGAGGTAGAATCCTTTGTCTATACCTCCATGAACATTGA
CTCGTGTGTTCAGAGCCTTTGGCCTCTCTGTGGAGTCTGGCTCTCTGGCT
CCTGTGCATTCTTTGAATAGTCACTCGTAAAAACTGTCAGTGCTTGAAAC
TGTTTCCTTTACTCATGTTGAAGGGACTTTGTTGGCTTTTAGAGTGTTGG
TCATGACTCCAAGAGCAGAGCAGGGAAGAGCCCAAGCATAGACTTGGTGC
CGTGGTGATGGCTGCAGTCCAGTTTTGTGATGCTGCTTTTACGTGTCCCT
CGATAACAGTCAGCTAGACACACTCAGGAGGACTACTGAGGCTCTGCGAC
CTTCAGGAGCTGAGCCTGCCTCTCTCCTTTAGATGACAGACCTTCATCTG
GGAACGTGCTGAGCCAGCACCCTCAGATGATTTCCCTCCAAACTGCTGAC
TAGGTCATCCTCTGTCTGGTAGAGACATTCACATCTTTGCTTTTATTCTA
TGCTCTCTGTACTTTTGACCAAAAATTGACCAAAGTAAGAAAATGCAAGT
TCTAAAAATAGACTAAGGATGCCTTTGCAGAACACCAAAGCATCCCAAGG
AACTGGTAGGGAAGTGGCGCCTGTCTCCTGGAGTGGAAGAGGCCTGCTCC
CTGGCTCTGGGTCTGCTGGGGGCACAGTAAATCAGTCTTGGCACCCACAT
CCAGGGCAGAGAGGTCTGTGGTTCTCAGCATCAGAAGGCAGCGCAGCCCC
TCTCCTCTTCAGGCTACAGGGTTGTCACCTGCTGAGTCCTCAGGTTGTTT
GGCCTCTCTGGTCCATCTTGGGCATTAGGTTCTCCAGCAGAGCTCTGGCC
AGCTGCCTCTTCTTTAACTGGGAACACAGGCTCTCACAAGATCAGAACCC
CCACTCACCCCCAAGATCTTATCTAGCAAGCCTGTAGTATTCAGTTTCTG
TTGTAGGAAGAGAGCGAGGCATCCCTGAATTCCACGCATCTGCTGGAAAC
GAGCCGTGTCAGATCGCACATCCCTGCGCCCCCATGCCCCTCTGAGTCAC
ACAGGACAGAGGAGGCAGAGCTTCTGCCCACTGTTATCTTCACTTTCTTT
GTCCAGTCTTTTGTTTTTAATAAGCAGTGACCCTCCCTACTCTTCTTTTT
AATGATTTTTGTAGTTGATTTGTCTGAACTGTGGCTACTGTGCATTCCTT
GAATAATCACTTGTAAAAATTGTCAGTGCTTGAAGCTGTTTCCTTTACTC
ACATTGAAGGGACTTCGTTGGTTTTTTGGAGTCTTGGTTGTGACTCCAAG
AGCAGAGTGAGGAAGACCCCCAAGCATAGACTCGGGTACTGTGATGATGG
CTGCAGTCCAGTTTTATGATTCTGCTTTTATGTGTCCCTTGATAACAGTG
ACTTAACAATATACATTCCTCATAAATAAAAAAAAAACAAGAATCTGAAT
TCTTAGAAA

SEQ ID NO: 19 - Human ADAR1 - Transcript Variant 6 Coding Sequence

ATGTCACCAATCTGCGACCAGACCATTGATTCCCGACTGAAGGGGTATTC
CCTCAGCGGATACTACACCCATCCATTTCAAGGCTATGAGCACAGACAGC
TCAGGTACCAGCAGCCTGGGCCAGGATCTTCCCCCAGTAGTTTCCTGCTT
AAGCAAATAGAATTTCTCAAGGGGCAGCTCCCAGAAGCACCGGTGATTGG
AAAGCAGACACCGTCACTGCCACCTTCCCTCCCAGGACTCCGGCCAAGGT
TTCCAGTACTACTTGCCTCCAGTACCAGAGGCAGGCAAGTGGACATCAGG
GGTGTCCCCAGGGGCGTGCATCTCAGAAGTCAGGGGCTCCAGAGAGGGTT
CCAGCATCCTTCACCACGTGGCAGGAGTCTGCCACAGAGAGGTGTTGATT
GCCTTTCCTCACATTTCCAGGAACTGAGTATCTACCAAGATCAGGAACAA
AGGATCTTAAAGTTCCTGGAAGAGCTTGGGGAAGGGAAGGCCACCACAGC
ACATGATCTGTCTGGGAAACTTGGGACTCCGAAGAAAGAAATCAATCGAG
TTTTATACTCCCTGGCAAAGAAGGGCAAGCTACAGAAAGAGGCAGGAACA
CCCCCTTTGTGGAAAATCGCGGTCTCCACTCAGGCTTGGAACCAGCACAG
CGGAGTGGTAAGACCAGACGGTCATAGCCAAGGAGCCCCAAACTCAGACC
CGAGTTTGGAACCGGAAGACAGAAACTCCACATCTGTCTCAGAAGATCTT
CTTGAGCCTTTTATTGCAGTCTCAGCTCAGGCTTGGAACCAGCACAGCGG
AGTGGTAAGACCAGACAGTCATAGCCAAGGATCCCCAAACTCAGACCCAG
GTTTGGAACCTGAAGACAGCAACTCCACATCTGCCTTGGAAGATCCTCTT
GAGTTTTTAGACATGGCCGAGATCAAGGAGAAAATCTGCGACTATCTCTT
CAATGTGTCTGACTCCTCTGCCCTGAATTTGGCTAAAAATATTGGCCTTA
CCAAGGCCCGAGATATAAATGCTGTGCTAATTGACATGGAAAGGCAGGGG
GATGTCTATAGACAAGGGACAACCCCTCCCATATGGCATTTGACAGACAA
GAAGCGAGAGAGGATGCAAATCAAGAGAAATACGAACAGTGTTCCTGAAA
CCGCTCCAGCTGCAATCCCTGAGACCAAAAGAAACGCAGAGTTCCTCACC
TGTAATATACCCACATCAAATGCCTCAAATAACATGGTAACCACAGAAAA
AGTGGAGAATGGGCAGGAACCTGTCATAAAGTTAGAAAACAGGCAAGAGG
CCAGACCAGAACCAGCAAGACTGAAACCACCTGTTCATTACAATGGCCCC
TCAAAAGCAGGGTATGTTGACTTTGAAAATGGCCAGTGGGCCACAGATGA
CATCCCAGATGACTTGAATAGTATCCGCGCAGCACCAGGTGAGTTTCGAG
CCATCATGGAGATGCCCTCCTTCTACAGTCATGGCTTGCCACGGTGTTCA
CCCTACAAGAAACTGACAGAGTGCCAGCTGAAGAACCCCATCAGCGGGCT
GTTAGAATATGCCCAGTTCGCTAGTCAAACCTGTGAGTTCAACATGATAG
AGCAGAGTGGACCACCCCATGAACCTCGATTTAAATTCCAGGTTGTCATC
AATGGCCGAGAGTTTCCCCCAGCTGAAGCTGGAAGCAAGAAAGTGGCCAA
GCAGGATGCAGCTATGAAAGCCATGACAATTCTGCTAGAGGAAGCCAAAG
CCAAGGACAGTGGAAAATCAGAAGAATCATCCCACTATTCCACAGAGAAA
GAATCAGAGAAGACTGCAGAGTCCCAGACCCCCACCCCTTCAGCCACATC
CTTCTTTTCTGGGAAGAGCCCCGTCACCACACTGCTTGAGTGTATGCACA
AATTGGGGAACTCCTGCGAATTCCGTCTCCTGTCCAAAGAAGGCCCTGCC
CATGAACCCAAGTTCCAATACTGTGTTGCAGTGGGAGCCCAAACTTTCCC
CAGTGTGAGTGCTCCCAGCAAGAAAGTGGCAAAGCAGATGGCCGCAGAGG
AAGCCATGAAGGCCCTGCATGGGGAGGCGACCAACTCCATGGCTTCTGAT
AACCAGCCTGAAGGTATGATCTCAGAGTCACTTGATAACTTGGAATCCAT
GATGCCCAACAAGGTCAGGAAGATTGGCGAGCTCGTGAGATACCTGAACA
CCAACCCTGTGGGTGGCCTTTTGGAGTACGCCCGCTCCCATGGCTTTGCT
GCTGAATTCAAGTTGGTCGACCAGTCCGGACCTCCTCACGAGCCCAAGTT
CGTTTACCAAGCAAAAGTTGGGGGTCGCTGGTTCCCAGCCGTCTGCGCAC
ACAGCAAGAAGCAAGGCAAGCAGGAAGCAGCAGATGCGGCTCTCCGTGTC
TTGATTGGGGAGAACGAGAAGGCAGAACGCATGGGTTTCACAGAGGTAAC
CCCAGTGACAGGGGCCAGTCTCAGAAGAACTATGCTCCTCCTCTCAAGGT
CCCCAGAAGCACAGCCAAAGACACTCCCTCTCACTGGCAGCACCTTCCAT
GACCAGATAGCCATGCTGAGCCACCGGTGCTTCAACACTCTGACTAACAG
CTTCCAGCCCTCCTTGCTCGGCCGCAAGATTCTGGCCGCCATCATTATGA
AAAAAGACTCTGAGGACATGGGTGTCGTCGTCAGCTTGGGAACAGGGAAT
CGCTGTGTGAAAGGAGATTCTCTCAGCCTAAAAGGAGAAACTGTCAATGA
CTGCCATGCAGAAATAATCTCCCGGAGAGGCTTCATCAGGTTTCTCTACA
GTGAGTTAATGAAATACAACTCCCAGACTGCGAAGGATAGTATATTTGAA
CCTGCTAAGGGAGGAGAAAAGCTCCAAATAAAAAAGACTGTGTCATTCCA
TCTGTATATCAGCACTGCTCCGTGTGGAGATGGCGCCCTCTTTGACAAGT
CCTGCAGCGACCGTGCTATGGAAAGCACAGAATCCCGCCACTACCCTGTC
TTCGAGAATCCCAAACAAGGAAAGCTCCGCACCAAGGTGGAGAACGGAGA
AGGCACAATCCCTGTGGAATCCAGTGACATTGTGCCTACGTGGGATGGCA
TTCGGCTCGGGGAGAGACTCCGTACCATGTCCTGTAGTGACAAAATCCTA
CGCTGGAACGTGCTGGGCCTGCAAGGGGCACTGTTGACCCACTTCCTGCA
GCCCATTTATCTCAAATCTGTCACATTGGGTTACCTTTTCAGCCAAGGGC
ATCTGACCCGTGCTATTTGCTGTCGTGTGACAAGAGATGGGAGTGCATTT
GAGGATGGACTACGACATCCCTTTATTGTCAACCACCCCAAGGTTGGCAG
AGTCAGCATATATGATTCCAAAAGGCAATCCGGGAAGACTAAGGAGACAA
GCGTCAACTGGTGTCTGGCTGATGGCTATGACCTGGAGATCCTGGACGGT
ACCAGAGGCACTGTGGATGGGCCACGGAATGAATTGTCCCGGGTCTCCAA
AAAGAACATTTTTCTTCTATTTAAGAAGCTCTGCTCCTTCCGTTACCGCA
GGGATCTACTGAGACTCTCCTATGGTGAGGCCAAGAAAGCTGCCCGTGAC
TACGAGACGGCCAAGAACTACTTCAAAAAAGGCCTGAAGGATATGGGCTA
TGGGAACTGGATTAGCAAACCCCAGGAGGAAAAGAACTTTTATCTCTGCC
CAGTATAG

SEQ ID NO: 20 - Human ADAR1 - Transcript Variant 6 Amino Acid Sequence (aka isoform-e)

MSPICDQTIDSRLKGYSLSGYYTHPFQGYEHRQLRYQQPGPGSSPSSFLL
KQIEFLKGQLPEAPVIGKQTPSLPPSLPGLRPRFPVLLASSTRGRQVDIR
GVPRGVHLRSQGLQRGFQHPSPRGRSLPQRGVDCLSSHFQELSIYQDQEQ
RILKFLEELGEGKATTAHDLSGKLGTPKKEINRVLYSLAKKGKLQKEAGT
PPLWKIAVSTQAWNQHSGVVRPDGHSQGAPNSDPSLEPEDRNSTSVSEDL
LEPFIAVSAQAWNQHSGVVRPDSHSQGSPNSDPGLEPEDSNSTSALEDPL
EFLDMAEIKEKICDYLFNVSDSSALNLAKNIGLTKARDINAVLIDMERQG
DVYRQGTTPPIWHLTDKKRERMQIKRNTNSVPETAPAAIPETKRNAEFLT
CNIPTSNASNNMVTTEKVENGQEPVIKLENRQEARPEPARLKPPVHYNGP
SKAGYVDFENGQWATDDIPDDLNSIRAAPGEFRAIMEMPSFYSHGLPRCS
PYKKLTECQLKNPISGLLEYAQFASQTCEFNMIEQSGPPHEPRFKFQVVI
NGREFPPAEAGSKKVAKQDAAMKAMTILLEEAKAKDSGKSEESSHYSTEK
ESEKTAESQTPTPSATSFFSGKSPVTTLLECMHKLGNSCEFRLLSKEGPA
HEPKFQYCVAVGAQTFPSVSAPSKKVAKQMAAEEAMKALHGEATNSMASD
NQPEGMISESLDNLESMMPNKVRKIGELVRYLNTNPVGGLLEYARSHGFA
AEFKLVDQSGPPHEPKFVYQAKVGGRWFPAVCAHSKKQGKQEAADAALRV
LIGENEKAERMGFTEVTPVTGASLRRTMLLLSRSPEAQPKTLPLTGSTFH
DQIAMLSHRCFNTLTNSFQPSLLGRKILAAIIMKKDSEDMGVVVSLGTGN
RCVKGDSLSLKGETVNDCHAEIISRRGFIRFLYSELMKYNSQTAKDSIFE
PAKGGEKLQIKKTVSFHLYISTAPCGDGALFDKSCSDRAMESTESRHYPV
FENPKQGKLRTKVENGEGTIPVESSDIVPTWDGIRLGERLRTMSCSDKIL
RWNVLGLQGALLTHFLQPIYLKSVTLGYLFSQGHLTRAICCRVTRDGSAF
EDGLRHPFIVNHPKVGRVSIYDSKRQSGKTKETSVNWCLADGYDLEILDG
TRGTVDGPRNELSRVSKKNIFLLFKKLCSFRYRRDLLRLSYGEAKKAARD
YETAKNY FKKGLKDMGYGNWISKPQEEKNFYLCPV

In certain embodiments, an ADAR1 polynucleotide or a human ADAR1 gene incorporated into a non-human animal (e.g., a rodent, e.g., a rat or mouse) is represented by or comprises a sequence encoding a human ADAR1 transcript variant 7 or a characteristic portion thereof. In certain embodiments, ADAR1 polynucleotide or a human ADAR1 gene comprises, or consists of, a nucleotide sequence that is the same as, differs by no more than 1, 2, 3, 4, 5, 6, 7, 8, 9, or 10 nucleotides from, or has at least has at least 90%, 92%, 94%, 96%, 98%, or 99% homology with, the nucleotide sequence of SEQ ID NO: 21 or a characteristic portion thereof. In some embodiments, an ADAR1 polynucleotide or a human ADAR1 gene comprises, or consists of, a nucleotide sequence that is the same as the nucleotide sequence of SEQ ID NO: 21 or a characteristic portion thereof. In certain embodiments, an encoded human ADAR1 Amino Acid sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the Amino Acid sequence of SEQ ID NO: 16 or a characteristic portion thereof. In certain embodiments, the amino acid sequence of an encoded and/or expressed ADAR1 polypeptide or a characteristic portion thereof, is or comprises a sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the amino acid sequence of SEQ ID NO: 16 or a characteristic portion thereof.

SEQ ID NO: 21 - Human ADAR1 - Transcript Variant 7 cDNA Sequence

TGATGTCACCAATCTGCGACCAGACCATTGATTCCCGACTGAAGGGGTAT
TCCCTCAGCGGATACTACACCCATCCATTTCAAGGCTATGAGCACAGACA
GCTCAGGTACCAGCAGCCTGGGCCAGGATCTTCCCCCAGTAGTTTCCTGC
TTAAGCAAATAGAATTTCTCAAGGGGCAGCTCCCAGAAGCACCGGTGATT
GGAAAGCAGACACCGTCACTGCCACCTTCCCTCCCAGGACTCCGGCCAAG
GTTTCCAGTACTACTTGCCTCCAGTACCAGAGGCAGGAACTGAGTATCTA
CCAAGATCAGGAACAAAGGATCTTAAAGTTCCTGGAAGAGCTTGGGGAAG
GGAAGGCCACCACAGCACATGATCTGTCTGGGAAACTTGGGACTCCGAAG
AAAGAAATCAATCGAGTTTTATACTCCCTGGCAAAGAAGGGCAAGCTACA
GAAAGAGGCAGGAACACCCCCTTTGTGGAAAATCGCGGTCTCCACTCAGG
CTTGGAACCAGCACAGCGGAGTGGTAAGACCAGACGGTCATAGCCAAGGA
GCCCCAAACTCAGACCCGAGTTTGGAACCGGAAGACAGAAACTCCACATC
TGTCTCAGAAGATCTTCTTGAGCCTTTTATTGCAGTCTCAGCTCAGGCTT
GGAACCAGCACAGCGGAGTGGTAAGACCAGACAGTCATAGCCAAGGATCC
CCAAACTCAGACCCAGGTTTGGAACCTGAAGACAGCAACTCCACATCTGC
CTTGGAAGATCCTCTTGAGTTTTTAGACATGGCCGAGATCAAGGAGAAAA
TCTGCGACTATCTCTTCAATGTGTCTGACTCCTCTGCCCTGAATTTGGCT
AAAAATATTGGCCTTACCAAGGCCCGAGATATAAATGCTGTGCTAATTGA
CATGGAAAGGCAGGGGGATGTCTATAGACAAGGGACAACCCCTCCCATAT
GGCATTTGACAGACAAGAAGCGAGAGAGGATGCAAATCAAGAGAAATACG
AACAGTGTTCCTGAAACCGCTCCAGCTGCAATCCCTGAGACCAAAAGAAA
CGCAGAGTTCCTCACCTGTAATATACCCACATCAAATGCCTCAAATAACA
TGGTAACCACAGAAAAAGTGGAGAATGGGCAGGAACCTGTCATAAAGTTA
GAAAACAGGCAAGAGGCCAGACCAGAACCAGCAAGACTGAAACCACCTGT
TCATTACAATGGCCCCTCAAAAGCAGGGTATGTTGACTTTGAAAATGGCC
AGTGGGCCACAGATGACATCCCAGATGACTTGAATAGTATCCGCGCAGCA
CCAGGTGAGTTTCGAGCCATCATGGAGATGCCCTCCTTCTACAGTCATGG
CTTGCCACGGTGTTCACCCTACAAGAAACTGACAGAGTGCCAGCTGAAGA
ACCCCATCAGCGGGCTGTTAGAATATGCCCAGTTCGCTAGTCAAACCTGT
GAGTTCAACATGATAGAGCAGAGTGGACCACCCCATGAACCTCGATTTAA
ATTCCAGGTTGTCATCAATGGCCGAGAGTTTCCCCCAGCTGAAGCTGGAA
GCAAGAAAGTGGCCAAGCAGGATGCAGCTATGAAAGCCATGACAATTCTG
CTAGAGGAAGCCAAAGCCAAGGACAGTGGAAAATCAGAAGAATCATCCCA
CTATTCCACAGAGAAAGAATCAGAGAAGACTGCAGAGTCCCAGACCCCCA
CCCCTTCAGCCACATCCTTCTTTTCTGGGAAGAGCCCCGTCACCACACTG
CTTGAGTGTATGCACAAATTGGGGAACTCCTGCGAATTCCGTCTCCTGTC
CAAAGAAGGCCCTGCCCATGAACCCAAGTTCCAATACTGTGTTGCAGTGG
GAGCCCAAACTTTCCCCAGTGTGAGTGCTCCCAGCAAGAAAGTGGCAAAG
CAGATGGCCGCAGAGGAAGCCATGAAGGCCCTGCATGGGGAGGCGACCAA
CTCCATGGCTTCTGATAACCAGCCTGAAGGTATGATCTCAGAGTCACTTG
ATAACTTGGAATCCATGATGCCCAACAAGGTCAGGAAGATTGGCGAGCTC
GTGAGATACCTGAACACCAACCCTGTGGGTGGCCTTTTGGAGTACGCCCG
CTCCCATGGCTTTGCTGCTGAATTCAAGTTGGTCGACCAGTCCGGACCTC
CTCACGAGCCCAAGTTCGTTTACCAAGCAAAAGTTGGGGGTCGCTGGTTC
CCAGCCGTCTGCGCACACAGCAAGAAGCAAGGCAAGCAGGAAGCAGCAGA
TGCGGCTCTCCGTGTCTTGATTGGGGAGAACGAGAAGGCAGAACGCATGG
GTTTCACAGAGGTAACCCCAGTGACAGGGGCCAGTCTCAGAAGAACTATG
CTCCTCCTCTCAAGGTCCCCAGAAGCACAGCCAAAGACACTCCCTCTCAC
TGGCAGCACCTTCCATGACCAGATAGCCATGCTGAGCCACCGGTGCTTCA
ACACTCTGACTAACAGCTTCCAGCCCTCCTTGCTCGGCCGCAAGATTCTG
GCCGCCATCATTATGAAAAAAGACTCTGAGGACATGGGTGTCGTCGTCAG
CTTGGGAACAGGGAATCGCTGTGTGAAAGGAGATTCTCTCAGCCTAAAAG
GAGAAACTGTCAATGACTGCCATGCAGAAATAATCTCCCGGAGAGGCTTC
ATCAGGTTTCTCTACAGTGAGTTAATGAAATACAACTCCCAGACTGCGAA
GGATAGTATATTTGAACCTGCTAAGGGAGGAGAAAAGCTCCAAATAAAAA
AGACTGTGTCATTCCATCTGTATATCAGCACTGCTCCGTGTGGAGATGGC
GCCCTCTTTGACAAGTCCTGCAGCGACCGTGCTATGGAAAGCACAGAATC
CCGCCACTACCCTGTCTTCGAGAATCCCAAACAAGGAAAGCTCCGCACCA
AGGTGGAGAACGGAGAAGGCACAATCCCTGTGGAATCCAGTGACATTGTG
CCTACGTGGGATGGCATTCGGCTCGGGGAGAGACTCCGTACCATGTCCTG
TAGTGACAAAATCCTACGCTGGAACGTGCTGGGCCTGCAAGGGGCACTGT
TGACCCACTTCCTGCAGCCCATTTATCTCAAATCTGTCACATTGGGTTAC
CTTTTCAGCCAAGGGCATCTGACCCGTGCTATTTGCTGTCGTGTGACAAG
AGATGGGAGTGCATTTGAGGATGGACTACGACATCCCTTTATTGTCAACC
ACCCCAAGGTTGGCAGAGTCAGCATATATGATTCCAAAAGGCAATCCGGG
AAGACTAAGGAGACAAGCGTCAACTGGTGTCTGGCTGATGGCTATGACCT
GGAGATCCTGGACGGTACCAGAGGCACTGTGGATGGGCCACGGAATGAAT
TGTCCCGGGTCTCCAAAAAGAACATTTTTCTTCTATTTAAGAAGCTCTGC
TCCTTCCGTTACCGCAGGGATCTACTGAGACTCTCCTATGGTGAGGCCAA
GAAAGCTGCCCGTGACTACGAGACGGCCAAGAACTACTTCAAAAAAGGCC
TGAAGGATATGGGCTATGGGAACTGGATTAGCAAACCCCAGGAGGAAAAG
AACTTTTATCTCTGCCCAGTATAGTATGCTCCAGTGACAGATGGATTAGG
GTGTGTCATACTAGGGTGTGAGAGAGGTAGGTCGTAGCATTCCTCATCAC
ATGGTCAGGGGATTTTTTTTTCTCCTTTTTTTTTCTTTTTAAGCCATAAT
TGGTGATACTGAAAACTTTGGGTTCCCATTTATCCTGCTTTCTTTGGGAT
TGCTAGGCAAGGTCTGGCCAGGCCCCCCTTTTTTCCCCCAAGTGAAGAGG
CAGAAACCTAAGAAGTTATCTTTTCTTTCTACCCAAAGCATACATAGTCA
CTGAGCACCTGCGGTCCATTTCCTCTTAAAAGTTTTGTTTTGATTTGTTT
CCATTTCCTTTCCCTTTGTGTTTGCTACACTGACCTCTTGCGGTCTTGAT
TAGGTTTCAGTCAACTCTGGATCATGTCAGGGACTGATAATTTCATTTGT
GGATTACGCAGACCCCTCTACTTCCCCTCTTTCCCTTCTGAGATTCTTTC
CTTGTGATCTGAATGTCTCCTTTTCCCCCTCAGAGGGCAAAGAGGTGAAC
ATAAAGGATTTGGTGAAACATTTGTAAGGGTAGGAGTTGAAAACTGCAGT
TCCCAGTGCCACGGAAGTGTGATTGGAGCCTGCAGATAATGCCCAGCCAT
CCTCCCATCCTGCACTTTAGCCAGCTGCAGGGCGGGCAAGGCAAGGAAAG
CTGCTTCCCTGGAAGTGTATCACTTTCTCCGGCAGCTGGGAAGTCTAGAA
CCAGCCAGACTGGGTTAAGGGAGCTGCTCAAGCAATAGCAGAGGTTTCAC
CCGGCAGGATGACACAGACCACTTCCCAGGGAGCACGGGCATGCCTTGGA
ATATTGCCAAGCTTCCAGCTGCCTCTTCTCCTAAAGCATTCCTAGGAATA
TTTTCCCCGCCAATGCTGGGCGTACACCCTAGCCAACGGGACAAATCCTA
GAGGGTATAAAATCATCTCTGCTCAGATAATCATGACTTAGCAAGAATAA
GGGCAAAAAATCCTGTTGGCTTAACGTCACTGTTCCACCCGGTGTAATAT
CTCTCATGACAGTGACACCAAGGGAAGTTGACTAAGTCACATGTAAATTA
GGAGTGTTTTAAAGAATGCCATAGATGTTGATTCTTAACTGCTACAGATA
ACCTGTAATTGAGCAGATTTAAAATTCAGGCATACTTTTCCATTTATCCA
AGTGCTTTCATTTTTCCAGATGGCTTCAGAAGTAGGCTCGTGGGCAGGGC
GCAGACCTGATCTTTATAGGGTTGACATAGAAAGCAGTAGTTGTGGGTGA
AAGGGCAGGTTGTCTTCAAACTCTGTGAGGTAGAATCCTTTGTCTATACC
TCCATGAACATTGACTCGTGTGTTCAGAGCCTTTGGCCTCTCTGTGGAGT
CTGGCTCTCTGGCTCCTGTGCATTCTTTGAATAGTCACTCGTAAAAACTG
TCAGTGCTTGAAACTGTTTCCTTTACTCATGTTGAAGGGACTTTGTTGGC
TTTTAGAGTGTTGGTCATGACTCCAAGAGCAGAGCAGGGAAGAGCCCAAG
CATAGACTTGGTGCCGTGGTGATGGCTGCAGTCCAGTTTTGTGATGCTGC
TTTTACGTGTCCCTCGATAACAGTCAGCTAGACACACTCAGGAGGACTAC
TGAGGCTCTGCGACCTTCAGGAGCTGAGCCTGCCTCTCTCCTTTAGATGA
CAGACCTTCATCTGGGAACGTGCTGAGCCAGCACCCTCAGATGATTTCCC
TCCAAACTGCTGACTAGGTCATCCTCTGTCTGGTAGAGACATTCACATCT
TTGCTTTTATTCTATGCTCTCTGTACTTTTGACCAAAAATTGACCAAAGT
AAGAAAATGCAAGTTCTAAAAATAGACTAAGGATGCCTTTGCAGAACACC
AAAGCATCCCAAGGAACTGGTAGGGAAGTGGCGCCTGTCTCCTGGAGTGG
AAGAGGCCTGCTCCCTGGCTCTGGGTCTGCTGGGGGCACAGTAAATCAGT
CTTGGCACCCACATCCAGGGCAGAGAGGTCTGTGGTTCTCAGCATCAGAA
GGCAGCGCAGCCCCTCTCCTCTTCAGGCTACAGGGTTGTCACCTGCTGAG
TCCTCAGGTTGTTTGGCCTCTCTGGTCCATCTTGGGCATTAGGTTCTCCA
GCAGAGCTCTGGCCAGCTGCCTCTTCTTTAACTGGGAACACAGGCTCTCA
CAAGATCAGAACCCCCACTCACCCCCAAGATCTTATCTAGCAAGCCTGTA
GTATTCAGTTTCTGTTGTAGGAAGAGAGCGAGGCATCCCTGAATTCCACG
CATCTGCTGGAAACGAGCCGTGTCAGATCGCACATCCCTGCGCCCCCATG
CCCCTCTGAGTCACACAGGACAGAGGAGGCAGAGCTTCTGCCCACTGTTA
TCTTCACTTTCTTTGTCCAGTCTTTTGTTTTTAATAAGCAGTGACCCTCC
CTACTCTTCTTTTTAATGATTTTTGTAGTTGATTTGTCTGAACTGTGGCT
ACTGTGCATTCCTTGAATAATCACTTGTAAAAATTGTCAGTGCTTGAAGC
TGTTTCCTTTACTCACATTGAAGGGACTTCGTTGGTTTTTTGGAGTCTTG
GTTGTGACTCCAAGAGCAGAGTGAGGAAGACCCCCAAGCATAGACTCGGG
TACTGTGATGATGGCTGCAGTCCAGTTTTATGATTCTGCTTTTATGTGTC
CCTTGATAACAGTGACTTAACAATATACATTCCTCATAAATAAAAAAAAA
ACAAGAATCTGAATTCTTAGAAA

In certain embodiments, an ADAR1 polynucleotide or a human ADAR1 gene incorporated into a non-human animal (e.g., a rodent, e.g., a rat or mouse) is represented by or comprises a sequence encoding a human ADAR1 transcript variant 8 or a characteristic portion thereof. In certain embodiments, an ADAR1 polynucleotide or a human ADAR1 gene comprises, or consists of, a nucleotide sequence that is the same as, differs by no more than 1, 2, 3, 4, 5, 6, 7, 8, 9, or 10 nucleotides from, or has at least has at least 90%, 92%, 94%, 96%, 98%, or 99% homology with, the nucleotide sequence of SEQ ID NO: 22 or a characteristic portion thereof. In some embodiments, an ADAR1 polynucleotide or a human ADAR1 gene comprises, or consists of, a nucleotide sequence that is the same as the nucleotide sequence of SEQ ID NO: 22 or a characteristic portion thereof. In certain embodiments, an encoded human ADAR1 Amino Acid sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the Amino Acid sequence of SEQ ID NO: 16 or a characteristic portion thereof. In certain embodiments, the amino acid sequence of an encoded and/or expressed ADAR1 polypeptide or a characteristic portion thereof, is or comprises a sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the amino acid sequence of SEQ ID NO: 16 or a characteristic portion thereof.

SEQ ID NO: 22 - Human ADAR1 - Transcript Variant 8 cDNA Sequence

GAACCGGAGCCATCTTGGGCCCGGCGCGCAGACCCGCGGAGTTTCCCGTG
CCGACGCCCCGGGGCCACTTCCAGTGCGGAGTAGCGGAGGCGTGGGGGCC
TCGAGGGGCTGGCGCGGCCCAGCGGTCGGGCCAGGGTCGTGCCGCCGGCG
GGTCGGGCCGGGCAATGCCTCGCGGGCGCAATGAATCCGCGGCAGGGGTA
TTCCCTCAGCGGATACTACACCCATCCATTTCAAGGCTATGAGCACAGAC
AGCTCAGGTACCAGCAGCCTGGGCCAGGATCTTCCCCCAGTAGTTTCCTG
CTTAAGCAAATAGAATTTCTCAAGGGGCAGCTCCCAGAAGCACCGGTGAT
TGGAAAGCAGACACCGTCACTGCCACCTTCCCTCCCAGGACTCCGGCCAA
GGTTTCCAGTACTACTTGCCTCCAGTACCAGAGGCAGGAACTGAGTATCT
ACCAAGATCAGGAACAAAGGATCTTAAAGTTCCTGGAAGAGCTTGGGGAA
GGGAAGGCCACCACAGCACATGATCTGTCTGGGAAACTTGGGACTCCGAA
GAAAGAAATCAATCGAGTTTTATACTCCCTGGCAAAGAAGGGCAAGCTAC
AGAAAGAGGCAGGAACACCCCCTTTGTGGAAAATCGCGGTCTCCACTCAG
GCTTGGAACCAGCACAGCGGAGTGGTAAGACCAGACGGTCATAGCCAAGG
AGCCCCAAACTCAGACCCGAGTTTGGAACCGGAAGACAGAAACTCCACAT
CTGTCTCAGAAGATCTTCTTGAGCCTTTTATTGCAGTCTCAGCTCAGGCT
TGGAACCAGCACAGCGGAGTGGTAAGACCAGACAGTCATAGCCAAGGATC
CCCAAACTCAGACCCAGGTTTGGAACCTGAAGACAGCAACTCCACATCTG
CCTTGGAAGATCCTCTTGAGTTTTTAGACATGGCCGAGATCAAGGAGAAA
ATCTGCGACTATCTCTTCAATGTGTCTGACTCCTCTGCCCTGAATTTGGC
TAAAAATATTGGCCTTACCAAGGCCCGAGATATAAATGCTGTGCTAATTG
ACATGGAAAGGCAGGGGGATGTCTATAGACAAGGGACAACCCCTCCCATA
TGGCATTTGACAGACAAGAAGCGAGAGAGGATGCAAATCAAGAGAAATAC
GAACAGTGTTCCTGAAACCGCTCCAGCTGCAATCCCTGAGACCAAAAGAA
ACGCAGAGTTCCTCACCTGTAATATACCCACATCAAATGCCTCAAATAAC
ATGGTAACCACAGAAAAAGTGGAGAATGGGCAGGAACCTGTCATAAAGTT
AGAAAACAGGCAAGAGGCCAGACCAGAACCAGCAAGACTGAAACCACCTG
TTCATTACAATGGCCCCTCAAAAGCAGGGTATGTTGACTTTGAAAATGGC
CAGTGGGCCACAGATGACATCCCAGATGACTTGAATAGTATCCGCGCAGC
ACCAGGTGAGTTTCGAGCCATCATGGAGATGCCCTCCTTCTACAGTCATG
GCTTGCCACGGTGTTCACCCTACAAGAAACTGACAGAGTGCCAGCTGAAG
AACCCCATCAGCGGGCTGTTAGAATATGCCCAGTTCGCTAGTCAAACCTG
TGAGTTCAACATGATAGAGCAGAGTGGACCACCCCATGAACCTCGATTTA
AATTCCAGGTTGTCATCAATGGCCGAGAGTTTCCCCCAGCTGAAGCTGGA
AGCAAGAAAGTGGCCAAGCAGGATGCAGCTATGAAAGCCATGACAATTCT
GCTAGAGGAAGCCAAAGCCAAGGACAGTGGAAAATCAGAAGAATCATCCC
ACTATTCCACAGAGAAAGAATCAGAGAAGACTGCAGAGTCCCAGACCCCC
ACCCCTTCAGCCACATCCTTCTTTTCTGGGAAGAGCCCCGTCACCACACT
GCTTGAGTGTATGCACAAATTGGGGAACTCCTGCGAATTCCGTCTCCTGT
CCAAAGAAGGCCCTGCCCATGAACCCAAGTTCCAATACTGTGTTGCAGTG
GGAGCCCAAACTTTCCCCAGTGTGAGTGCTCCCAGCAAGAAAGTGGCAAA
GCAGATGGCCGCAGAGGAAGCCATGAAGGCCCTGCATGGGGAGGCGACCA
ACTCCATGGCTTCTGATAACCAGCCTGAAGGTATGATCTCAGAGTCACTT
GATAACTTGGAATCCATGATGCCCAACAAGGTCAGGAAGATTGGCGAGCT
CGTGAGATACCTGAACACCAACCCTGTGGGTGGCCTTTTGGAGTACGCCC
GCTCCCATGGCTTTGCTGCTGAATTCAAGTTGGTCGACCAGTCCGGACCT
CCTCACGAGCCCAAGTTCGTTTACCAAGCAAAAGTTGGGGGTCGCTGGTT
CCCAGCCGTCTGCGCACACAGCAAGAAGCAAGGCAAGCAGGAAGCAGCAG
ATGCGGCTCTCCGTGTCTTGATTGGGGAGAACGAGAAGGCAGAACGCATG
GGTTTCACAGAGGTAACCCCAGTGACAGGGGCCAGTCTCAGAAGAACTAT
GCTCCTCCTCTCAAGGTCCCCAGAAGCACAGCCAAAGACACTCCCTCTCA
CTGGCAGCACCTTCCATGACCAGATAGCCATGCTGAGCCACCGGTGCTTC
AACACTCTGACTAACAGCTTCCAGCCCTCCTTGCTCGGCCGCAAGATTCT
GGCCGCCATCATTATGAAAAAAGACTCTGAGGACATGGGTGTCGTCGTCA
GCTTGGGAACAGGGAATCGCTGTGTGAAAGGAGATTCTCTCAGCCTAAAA
GGAGAAACTGTCAATGACTGCCATGCAGAAATAATCTCCCGGAGAGGCTT
CATCAGGTTTCTCTACAGTGAGTTAATGAAATACAACTCCCAGACTGCGA
AGGATAGTATATTTGAACCTGCTAAGGGAGGAGAAAAGCTCCAAATAAAA
AAGACTGTGTCATTCCATCTGTATATCAGCACTGCTCCGTGTGGAGATGG
CGCCCTCTTTGACAAGTCCTGCAGCGACCGTGCTATGGAAAGCACAGAAT
CCCGCCACTACCCTGTCTTCGAGAATCCCAAACAAGGAAAGCTCCGCACC
AAGGTGGAGAACGGAGAAGGCACAATCCCTGTGGAATCCAGTGACATTGT
GCCTACGTGGGATGGCATTCGGCTCGGGGAGAGACTCCGTACCATGTCCT
GTAGTGACAAAATCCTACGCTGGAACGTGCTGGGCCTGCAAGGGGCACTG
TTGACCCACTTCCTGCAGCCCATTTATCTCAAATCTGTCACATTGGGTTA
CCTTTTCAGCCAAGGGCATCTGACCCGTGCTATTTGCTGTCGTGTGACAA
GAGATGGGAGTGCATTTGAGGATGGACTACGACATCCCTTTATTGTCAAC
CACCCCAAGGTTGGCAGAGTCAGCATATATGATTCCAAAAGGCAATCCGG
GAAGACTAAGGAGACAAGCGTCAACTGGTGTCTGGCTGATGGCTATGACC
TGGAGATCCTGGACGGTACCAGAGGCACTGTGGATGGGCCACGGAATGAA
TTGTCCCGGGTCTCCAAAAAGAACATTTTTCTTCTATTTAAGAAGCTCTG
CTCCTTCCGTTACCGCAGGGATCTACTGAGACTCTCCTATGGTGAGGCCA
AGAAAGCTGCCCGTGACTACGAGACGGCCAAGAACTACTTCAAAAAAGGC
CTGAAGGATATGGGCTATGGGAACTGGATTAGCAAACCCCAGGAGGAAAA
GAACTTTTATCTCTGCCCAGTATAGTATGCTCCAGTGACAGATGGATTAG
GGTGTGTCATACTAGGGTGTGAGAGAGGTAGGTCGTAGCATTCCTCATCA
CATGGTCAGGGGATTTTTTTTTCTCCTTTTTTTTTCTTTTTAAGCCATAA
TTGGTGATACTGAAAACTTTGGGTTCCCATTTATCCTGCTTTCTTTGGGA
TTGCTAGGCAAGGTCTGGCCAGGCCCCCCTTTTTTCCCCCAAGTGAAGAG
GCAGAAACCTAAGAAGTTATCTTTTCTTTCTACCCAAAGCATACATAGTC
ACTGAGCACCTGCGGTCCATTTCCTCTTAAAAGTTTTGTTTTGATTTGTT
TCCATTTCCTTTCCCTTTGTGTTTGCTACACTGACCTCTTGCGGTCTTGA
TTAGGTTTCAGTCAACTCTGGATCATGTCAGGGACTGATAATTTCATTTG
TGGATTACGCAGACCCCTCTACTTCCCCTCTTTCCCTTCTGAGATTCTTT
CCTTGTGATCTGAATGTCTCCTTTTCCCCCTCAGAGGGCAAAGAGGTGAA
CATAAAGGATTTGGTGAAACATTTGTAAGGGTAGGAGTTGAAAACTGCAG
TTCCCAGTGCCACGGAAGTGTGATTGGAGCCTGCAGATAATGCCCAGCCA
TCCTCCCATCCTGCACTTTAGCCAGCTGCAGGGCGGGCAAGGCAAGGAAA
GCTGCTTCCCTGGAAGTGTATCACTTTCTCCGGCAGCTGGGAAGTCTAGA
ACCAGCCAGACTGGGTTAAGGGAGCTGCTCAAGCAATAGCAGAGGTTTCA
CCCGGCAGGATGACACAGACCACTTCCCAGGGAGCACGGGCATGCCTTGG
AATATTGCCAAGCTTCCAGCTGCCTCTTCTCCTAAAGCATTCCTAGGAAT
ATTTTCCCCGCCAATGCTGGGCGTACACCCTAGCCAACGGGACAAATCCT
AGAGGGTATAAAATCATCTCTGCTCAGATAATCATGACTTAGCAAGAATA
AGGGCAAAAAATCCTGTTGGCTTAACGTCACTGTTCCACCCGGTGTAATA
TCTCTCATGACAGTGACACCAAGGGAAGTTGACTAAGTCACATGTAAATT
AGGAGTGTTTTAAAGAATGCCATAGATGTTGATTCTTAACTGCTACAGAT
AACCTGTAATTGAGCAGATTTAAAATTCAGGCATACTTTTCCATTTATCC
AAGTGCTTTCATTTTTCCAGATGGCTTCAGAAGTAGGCTCGTGGGCAGGG
CGCAGACCTGATCTTTATAGGGTTGACATAGAAAGCAGTAGTTGTGGGTG
AAAGGGCAGGTTGTCTTCAAACTCTGTGAGGTAGAATCCTTTGTCTATAC
CTCCATGAACATTGACTCGTGTGTTCAGAGCCTTTGGCCTCTCTGTGGAG
TCTGGCTCTCTGGCTCCTGTGCATTCTTTGAATAGTCACTCGTAAAAACT
GTCAGTGCTTGAAACTGTTTCCTTTACTCATGTTGAAGGGACTTTGTTGG
CTTTTAGAGTGTTGGTCATGACTCCAAGAGCAGAGCAGGGAAGAGCCCAA
GCATAGACTTGGTGCCGTGGTGATGGCTGCAGTCCAGTTTTGTGATGCTG
CTTTTACGTGTCCCTCGATAACAGTCAGCTAGACACACTCAGGAGGACTA
CTGAGGCTCTGCGACCTTCAGGAGCTGAGCCTGCCTCTCTCCTTTAGATG
ACAGACCTTCATCTGGGAACGTGCTGAGCCAGCACCCTCAGATGATTTCC
CTCCAAACTGCTGACTAGGTCATCCTCTGTCTGGTAGAGACATTCACATC
TTTGCTTTTATTCTATGCTCTCTGTACTTTTGACCAAAAATTGACCAAAG
TAAGAAAATGCAAGTTCTAAAAATAGACTAAGGATGCCTTTGCAGAACAC
CAAAGCATCCCAAGGAACTGGTAGGGAAGTGGCGCCTGTCTCCTGGAGTG
GAAGAGGCCTGCTCCCTGGCTCTGGGTCTGCTGGGGGCACAGTAAATCAG
TCTTGGCACCCACATCCAGGGCAGAGAGGTCTGTGGTTCTCAGCATCAGA
AGGCAGCGCAGCCCCTCTCCTCTTCAGGCTACAGGGTTGTCACCTGCTGA
GTCCTCAGGTTGTTTGGCCTCTCTGGTCCATCTTGGGCATTAGGTTCTCC
AGCAGAGCTCTGGCCAGCTGCCTCTTCTTTAACTGGGAACACAGGCTCTC
ACAAGATCAGAACCCCCACTCACCCCCAAGATCTTATCTAGCAAGCCTGT
AGTATTCAGTTTCTGTTGTAGGAAGAGAGCGAGGCATCCCTGAATTCCAC
GCATCTGCTGGAAACGAGCCGTGTCAGATCGCACATCCCTGCGCCCCCAT
GCCCCTCTGAGTCACACAGGACAGAGGAGGCAGAGCTTCTGCCCACTGTT
ATCTTCACTTTCTTTGTCCAGTCTTTTGTTTTTAATAAGCAGTGACCCTC
CCTACTCTTCTTTTTAATGATTTTTGTAGTTGATTTGTCTGAACTGTGGC
TACTGTGCATTCCTTGAATAATCACTTGTAAAAATTGTCAGTGCTTGAAG
CTGTTTCCTTTACTCACATTGAAGGGACTTCGTTGGTTTTTTGGAGTCTT
GGTTGTGACTCCAAGAGCAGAGTGAGGAAGACCCCCAAGCATAGACTCGG
GTACTGTGATGATGGCTGCAGTCCAGTTTTATGATTCTGCTTTTATGTGT
CCCTTGATAACAGTGACTTAACAATATACATTCCTCATAAATAAAAAAAA
AACAAGAATCTGAATTCTTAGAAA

In certain embodiments, an ADAR1 polynucleotide or a human ADAR1 gene incorporated into a non-human animal (e.g., a rodent, e.g., a rat or mouse) is represented by or comprises a sequence encoding a human ADAR1 transcript variant 9 or a characteristic portion thereof. In certain embodiments, an ADAR1 polynucleotide or a human ADAR1 gene comprises, or consists of, a nucleotide sequence that is the same as, differs by no more than 1, 2, 3, 4, 5, 6, 7, 8, 9, or 10 nucleotides from, or has at least has at least 90%, 92%, 94%, 96%, 98%, or 99% homology with, the nucleotide sequence of SEQ ID NO: 23 or a characteristic portion thereof. In some embodiments, an ADAR1 polynucleotide or a human ADAR1 gene comprises, or consists of, a nucleotide sequence that is the same as the nucleotide sequence of SEQ ID NO: 23 or a characteristic portion thereof. In certain embodiments, an encoded human ADAR1 Amino Acid sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the Amino Acid sequence of SEQ ID NO: 16 or a characteristic portion thereof. In certain embodiments, the amino acid sequence of an encoded and/or expressed ADAR1 polypeptide or a characteristic portion thereof, is or comprises a sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the amino acid sequence of SEQ ID NO: 16 or a characteristic portion thereof.

SEQ ID NO: 23 - Human ADAR1 - Transcript Variant 9 cDNA Sequence

GGCGCGGAGAGAGGGAGGACCGGGCGTCATGCTGTTTCTGGCCTGAGGGG
TATTCCCTCAGCGGATACTACACCCATCCATTTCAAGGCTATGAGCACAG
ACAGCTCAGGTACCAGCAGCCTGGGCCAGGATCTTCCCCCAGTAGTTTCC
TGCTTAAGCAAATAGAATTTCTCAAGGGGCAGCTCCCAGAAGCACCGGTG
ATTGGAAAGCAGACACCGTCACTGCCACCTTCCCTCCCAGGACTCCGGCC
AAGGTTTCCAGTACTACTTGCCTCCAGTACCAGAGGCAGGCAAGTGGACA
TCAGGGGTGTCCCCAGGGGCGTGCATCTCAGAAGTCAGGGGCTCCAGAGA
GGGTTCCAGCATCCTTCACCACGTGGCAGGAGTCTGCCACAGAGAGGTGT
TGATTGCCTTTCCTCACATTTCCAGGAACTGAGTATCTACCAAGATCAGG
AACAAAGGATCTTAAAGTTCCTGGAAGAGCTTGGGGAAGGGAAGGCCACC
ACAGCACATGATCTGTCTGGGAAACTTGGGACTCCGAAGAAAGAAATCAA
TCGAGTTTTATACTCCCTGGCAAAGAAGGGCAAGCTACAGAAAGAGGCAG
GAACACCCCCTTTGTGGAAAATCGCGGTCTCCACTCAGGCTTGGAACCAG
CACAGCGGAGTGGTAAGACCAGACGGTCATAGCCAAGGAGCCCCAAACTC
AGACCCGAGTTTGGAACCGGAAGACAGAAACTCCACATCTGTCTCAGAAG
ATCTTCTTGAGCCTTTTATTGCAGTCTCAGCTCAGGCTTGGAACCAGCAC
AGCGGAGTGGTAAGACCAGACAGTCATAGCCAAGGATCCCCAAACTCAGA
CCCAGGTTTGGAACCTGAAGACAGCAACTCCACATCTGCCTTGGAAGATC
CTCTTGAGTTTTTAGACATGGCCGAGATCAAGGAGAAAATCTGCGACTAT
CTCTTCAATGTGTCTGACTCCTCTGCCCTGAATTTGGCTAAAAATATTGG
CCTTACCAAGGCCCGAGATATAAATGCTGTGCTAATTGACATGGAAAGGC
AGGGGGATGTCTATAGACAAGGGACAACCCCTCCCATATGGCATTTGACA
GACAAGAAGCGAGAGAGGATGCAAATCAAGAGAAATACGAACAGTGTTCC
TGAAACCGCTCCAGCTGCAATCCCTGAGACCAAAAGAAACGCAGAGTTCC
TCACCTGTAATATACCCACATCAAATGCCTCAAATAACATGGTAACCACA
GAAAAAGTGGAGAATGGGCAGGAACCTGTCATAAAGTTAGAAAACAGGCA
AGAGGCCAGACCAGAACCAGCAAGACTGAAACCACCTGTTCATTACAATG
GCCCCTCAAAAGCAGGGTATGTTGACTTTGAAAATGGCCAGTGGGCCACA
GATGACATCCCAGATGACTTGAATAGTATCCGCGCAGCACCAGGTGAGTT
TCGAGCCATCATGGAGATGCCCTCCTTCTACAGTCATGGCTTGCCACGGT
GTTCACCCTACAAGAAACTGACAGAGTGCCAGCTGAAGAACCCCATCAGC
GGGCTGTTAGAATATGCCCAGTTCGCTAGTCAAACCTGTGAGTTCAACAT
GATAGAGCAGAGTGGACCACCCCATGAACCTCGATTTAAATTCCAGGTTG
TCATCAATGGCCGAGAGTTTCCCCCAGCTGAAGCTGGAAGCAAGAAAGTG
GCCAAGCAGGATGCAGCTATGAAAGCCATGACAATTCTGCTAGAGGAAGC
CAAAGCCAAGGACAGTGGAAAATCAGAAGAATCATCCCACTATTCCACAG
AGAAAGAATCAGAGAAGACTGCAGAGTCCCAGACCCCCACCCCTTCAGCC
ACATCCTTCTTTTCTGGGAAGAGCCCCGTCACCACACTGCTTGAGTGTAT
GCACAAATTGGGGAACTCCTGCGAATTCCGTCTCCTGTCCAAAGAAGGCC
CTGCCCATGAACCCAAGTTCCAATACTGTGTTGCAGTGGGAGCCCAAACT
TTCCCCAGTGTGAGTGCTCCCAGCAAGAAAGTGGCAAAGCAGATGGCCGC
AGAGGAAGCCATGAAGGCCCTGCATGGGGAGGCGACCAACTCCATGGCTT
CTGATAACCAGCCTGAAGGTATGATCTCAGAGTCACTTGATAACTTGGAA
TCCATGATGCCCAACAAGGTCAGGAAGATTGGCGAGCTCGTGAGATACCT
GAACACCAACCCTGTGGGTGGCCTTTTGGAGTACGCCCGCTCCCATGGCT
TTGCTGCTGAATTCAAGTTGGTCGACCAGTCCGGACCTCCTCACGAGCCC
AAGTTCGTTTACCAAGCAAAAGTTGGGGGTCGCTGGTTCCCAGCCGTCTG
CGCACACAGCAAGAAGCAAGGCAAGCAGGAAGCAGCAGATGCGGCTCTCC
GTGTCTTGATTGGGGAGAACGAGAAGGCAGAACGCATGGGTTTCACAGAG
GTAACCCCAGTGACAGGGGCCAGTCTCAGAAGAACTATGCTCCTCCTCTC
AAGGTCCCCAGAAGCACAGCCAAAGACACTCCCTCTCACTGGCAGCACCT
TCCATGACCAGATAGCCATGCTGAGCCACCGGTGCTTCAACACTCTGACT
AACAGCTTCCAGCCCTCCTTGCTCGGCCGCAAGATTCTGGCCGCCATCAT
TATGAAAAAAGACTCTGAGGACATGGGTGTCGTCGTCAGCTTGGGAACAG
GGAATCGCTGTGTGAAAGGAGATTCTCTCAGCCTAAAAGGAGAAACTGTC
AATGACTGCCATGCAGAAATAATCTCCCGGAGAGGCTTCATCAGGTTTCT
CTACAGTGAGTTAATGAAATACAACTCCCAGACTGCGAAGGATAGTATAT
TTGAACCTGCTAAGGGAGGAGAAAAGCTCCAAATAAAAAAGACTGTGTCA
TTCCATCTGTATATCAGCACTGCTCCGTGTGGAGATGGCGCCCTCTTTGA
CAAGTCCTGCAGCGACCGTGCTATGGAAAGCACAGAATCCCGCCACTACC
CTGTCTTCGAGAATCCCAAACAAGGAAAGCTCCGCACCAAGGTGGAGAAC
GGAGAAGGCACAATCCCTGTGGAATCCAGTGACATTGTGCCTACGTGGGA
TGGCATTCGGCTCGGGGAGAGACTCCGTACCATGTCCTGTAGTGACAAAA
TCCTACGCTGGAACGTGCTGGGCCTGCAAGGGGCACTGTTGACCCACTTC
CTGCAGCCCATTTATCTCAAATCTGTCACATTGGGTTACCTTTTCAGCCA
AGGGCATCTGACCCGTGCTATTTGCTGTCGTGTGACAAGAGATGGGAGTG
CATTTGAGGATGGACTACGACATCCCTTTATTGTCAACCACCCCAAGGTT
GGCAGAGTCAGCATATATGATTCCAAAAGGCAATCCGGGAAGACTAAGGA
GACAAGCGTCAACTGGTGTCTGGCTGATGGCTATGACCTGGAGATCCTGG
ACGGTACCAGAGGCACTGTGGATGGGCCACGGAATGAATTGTCCCGGGTC
TCCAAAAAGAACATTTTTCTTCTATTTAAGAAGCTCTGCTCCTTCCGTTA
CCGCAGGGATCTACTGAGACTCTCCTATGGTGAGGCCAAGAAAGCTGCCC
GTGACTACGAGACGGCCAAGAACTACTTCAAAAAAGGCCTGAAGGATATG
GGCTATGGGAACTGGATTAGCAAACCCCAGGAGGAAAAGAACTTTTATCT
CTGCCCAGTATAGTATGCTCCAGTGACAGATGGATTAGGGTGTGTCATAC
TAGGGTGTGAGAGAGGTAGGTCGTAGCATTCCTCATCACATGGTCAGGGG
ATTTTTTTTTCTCCTTTTTTTTTCTTTTTAAGCCATAATTGGTGATACTG
AAAACTTTGGGTTCCCATTTATCCTGCTTTCTTTGGGATTGCTAGGCAAG
GTCTGGCCAGGCCCCCCTTTTTTCCCCCAAGTGAAGAGGCAGAAACCTAA
GAAGTTATCTTTTCTTTCTACCCAAAGCATACATAGTCACTGAGCACCTG
CGGTCCATTTCCTCTTAAAAGTTTTGTTTTGATTTGTTTCCATTTCCTTT
CCCTTTGTGTTTGCTACACTGACCTCTTGCGGTCTTGATTAGGTTTCAGT
CAACTCTGGATCATGTCAGGGACTGATAATTTCATTTGTGGATTACGCAG
ACCCCTCTACTTCCCCTCTTTCCCTTCTGAGATTCTTTCCTTGTGATCTG
AATGTCTCCTTTTCCCCCTCAGAGGGCAAAGAGGTGAACATAAAGGATTT
GGTGAAACATTTGTAAGGGTAGGAGTTGAAAACTGCAGTTCCCAGTGCCA
CGGAAGTGTGATTGGAGCCTGCAGATAATGCCCAGCCATCCTCCCATCCT
GCACTTTAGCCAGCTGCAGGGCGGGCAAGGCAAGGAAAGCTGCTTCCCTG
GAAGTGTATCACTTTCTCCGGCAGCTGGGAAGTCTAGAACCAGCCAGACT
GGGTTAAGGGAGCTGCTCAAGCAATAGCAGAGGTTTCACCCGGCAGGATG
ACACAGACCACTTCCCAGGGAGCACGGGCATGCCTTGGAATATTGCCAAG
CTTCCAGCTGCCTCTTCTCCTAAAGCATTCCTAGGAATATTTTCCCCGCC
AATGCTGGGCGTACACCCTAGCCAACGGGACAAATCCTAGAGGGTATAAA
ATCATCTCTGCTCAGATAATCATGACTTAGCAAGAATAAGGGCAAAAAAT
CCTGTTGGCTTAACGTCACTGTTCCACCCGGTGTAATATCTCTCATGACA
GTGACACCAAGGGAAGTTGACTAAGTCACATGTAAATTAGGAGTGTTTTA
AAGAATGCCATAGATGTTGATTCTTAACTGCTACAGATAACCTGTAATTG
AGCAGATTTAAAATTCAGGCATACTTTTCCATTTATCCAAGTGCTTTCAT
TTTTCCAGATGGCTTCAGAAGTAGGCTCGTGGGCAGGGCGCAGACCTGAT
CTTTATAGGGTTGACATAGAAAGCAGTAGTTGTGGGTGAAAGGGCAGGTT
GTCTTCAAACTCTGTGAGGTAGAATCCTTTGTCTATACCTCCATGAACAT
TGACTCGTGTGTTCAGAGCCTTTGGCCTCTCTGTGGAGTCTGGCTCTCTG
GCTCCTGTGCATTCTTTGAATAGTCACTCGTAAAAACTGTCAGTGCTTGA
AACTGTTTCCTTTACTCATGTTGAAGGGACTTTGTTGGCTTTTAGAGTGT
TGGTCATGACTCCAAGAGCAGAGCAGGGAAGAGCCCAAGCATAGACTTGG
TGCCGTGGTGATGGCTGCAGTCCAGTTTTGTGATGCTGCTTTTACGTGTC
CCTCGATAACAGTCAGCTAGACACACTCAGGAGGACTACTGAGGCTCTGC
GACCTTCAGGAGCTGAGCCTGCCTCTCTCCTTTAGATGACAGACCTTCAT
CTGGGAACGTGCTGAGCCAGCACCCTCAGATGATTTCCCTCCAAACTGCT
GACTAGGTCATCCTCTGTCTGGTAGAGACATTCACATCTTTGCTTTTATT
CTATGCTCTCTGTACTTTTGACCAAAAATTGACCAAAGTAAGAAAATGCA
AGTTCTAAAAATAGACTAAGGATGCCTTTGCAGAACACCAAAGCATCCCA
AGGAACTGGTAGGGAAGTGGCGCCTGTCTCCTGGAGTGGAAGAGGCCTGC
TCCCTGGCTCTGGGTCTGCTGGGGGCACAGTAAATCAGTCTTGGCACCCA
CATCCAGGGCAGAGAGGTCTGTGGTTCTCAGCATCAGAAGGCAGCGCAGC
CCCTCTCCTCTTCAGGCTACAGGGTTGTCACCTGCTGAGTCCTCAGGTTG
TTTGGCCTCTCTGGTCCATCTTGGGCATTAGGTTCTCCAGCAGAGCTCTG
GCCAGCTGCCTCTTCTTTAACTGGGAACACAGGCTCTCACAAGATCAGAA
CCCCCACTCACCCCCAAGATCTTATCTAGCAAGCCTGTAGTATTCAGTTT
CTGTTGTAGGAAGAGAGCGAGGCATCCCTGAATTCCACGCATCTGCTGGA
AACGAGCCGTGTCAGATCGCACATCCCTGCGCCCCCATGCCCCTCTGAGT
CACACAGGACAGAGGAGGCAGAGCTTCTGCCCACTGTTATCTTCACTTTC
TTTGTCCAGTCTTTTGTTTTTAATAAGCAGTGACCCTCCCTACTCTTCTT
TTTAATGATTTTTGTAGTTGATTTGTCTGAACTGTGGCTACTGTGCATTC
CTTGAATAATCACTTGTAAAAATTGTCAGTGCTTGAAGCTGTTTCCTTTA
CTCACATTGAAGGGACTTCGTTGGTTTTTTGGAGTCTTGGTTGTGACTCC
AAGAGCAGAGTGAGGAAGACCCCCAAGCATAGACTCGGGTACTGTGATGA
TGGCTGCAGTCCAGTTTTATGATTCTGCTTTTATGTGTCCCTTGATAACA
GTGACTTAACAATATACATTCCTCATAAATAAAAAAAAAACAAGAATCTG
AATTCTTAGAAA

In certain embodiments, an ADAR1 polynucleotide or a human ADAR1 gene incorporated into a non-human animal (e.g., a rodent, e.g., a rat or mouse) is represented by or comprises a sequence encoding a human ADAR1 transcript variant 10 or a characteristic portion thereof. In certain embodiments, an ADAR1 polynucleotide or a human ADAR1 gene comprises, or consists of, a nucleotide sequence that is the same as, differs by no more than 1, 2, 3, 4, 5, 6, 7, 8, 9, or 10 nucleotides from, or has at least has at least 90%, 92%, 94%, 96%, 98%, or 99% homology with, the nucleotide sequence of SEQ ID NO: 24 or SEQ ID NO: 25 or a characteristic portion thereof. In some embodiments, an ADAR1 polynucleotide or a human ADAR1 gene comprises, or consists of, a nucleotide sequence that is the same as the nucleotide sequence of SEQ ID NO: 24 or SEQ ID NO: 25 or a characteristic portion thereof. In certain embodiments, an encoded human ADAR1 Amino Acid sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the Amino Acid sequence of SEQ ID NO: 26 or a characteristic portion thereof. In certain embodiments, the amino acid sequence of an encoded and/or expressed ADAR1 polypeptide or a characteristic portion thereof, is or comprises a sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the amino acid sequence of SEQ ID NO: 26 or a characteristic portion thereof.

SEQ ID NO: 24 - Human ADAR1 - Transcript Variant 10 cDNA Sequence

AACCGGAGCCATCTTGGGCCCGGCGCGCAGACCCGCGGAGTTTCCCGTGC
CGACGCCCCGGGGCCACTTCCAGTGCGGAGTAGCGGAGGCGTGGGGGCCT
CGAGGGGCTGGCGCGGCCCAGCGGTCGGGCCAGGGTCGTGCCGCCGGCGG
GTCGGGCCGGGCAATGCCTCGCGGGCGCAATGAATCCGCGGCAGGGGTAT
TCCCTCAGCGGATACTACACCCATCCATTTCAAGGCTATGAGCACAGACA
GCTCAGGTACCAGCAGCCTGGGCCAGGATCTTCCCCCAGTAGTTTCCTGC
TTAAGCAAATAGAATTTCTCAAGGGGCAGCTCCCAGAAGCACCGGTGATT
GGAAAGCAGACACCGTCACTGCCACCTTCCCTCCCAGGACTCCGGCCAAG
GTTTCCAGTACTACTTGCCTCCAGTACCAGAGGCAGGAACTGAGTATCTA
CCAAGATCAGGAACAAAGGATCTTAAAGTTCCTGGAAGAGCTTGGGGAAG
GGAAGGCCACCACAGCACATGATCTGTCTGGGAAACTTGGGACTCCGAAG
AAAGAAATCAATCGAGTTTTATACTCCCTGGCAAAGAAGGGCAAGCTACA
GAAAGAGGCAGGAACACCCCCTTTGTGGAAAATCGCGGTCTCCACTCAGG
CTTGGAACCAGCACAGCGGAGTGGTAAGACCAGACGGTCATAGCCAAGGA
GCCCCAAACTCAGACCCGAGTTTGGAACCGGAAGACAGAAACTCCACATC
TGTCTCAGAAGATCTTCTTGAGCCTTTTATTGCAGTCTCAGCTCAGGCTT
GGAACCAGCACAGCGGAGTGGTAAGACCAGACAGTCATAGCCAAGGATCC
CCAAACTCAGACCCAGGTTTGGAACCTGAAGACAGCAACTCCACATCTGC
CTTGGAAGATCCTCTTGAGTTTTTAGACATGGCCGAGATCAAGGAGAAAA
TCTGCGACTATCTCTTCAATGTGTCTGACTCCTCTGCCCTGAATTTGGCT
AAAAATATTGGCCTTACCAAGGCCCGAGATATAAATGCTGTGCTAATTGA
CATGGAAAGGCAGGGGGATGTCTATAGACAAGGGACAACCCCTCCCATAT
GGCATTTGACAGACAAGAAGCGAGAGAGGATGCAAATCAAGAGAAATACG
AACAGTGTTCCTGAAACCGCTCCAGCTGCAATCCCTGAGACCAAAAGAAA
CGCAGAGTTCCTCACCTGTAATATACCCACATCAAATGCCTCAAATAACA
TGGTAACCACAGAAAAAGTGGAGAATGGGCAGGAACCTGTCATAAAGTTA
GAAAACAGGCAAGAGGCCAGACCAGAACCAGCAAGACTGAAACCACCTGT
TCATTACAATGGCCCCTCAAAAGCAGGGTATGTTGACTTTGAAAATGGCC
AGTGGGCCACAGATGACATCCCAGATGACTTGAATAGTATCCGCGCAGCA
CCAGGTGAGTTTCGAGCCATCATGGAGATGCCCTCCTTCTACAGTCATGG
CTTGCCACGGTGTTCACCCTACAAGAAACTGACAGAGTGCCAGCTGAAGA
ACCCCATCAGCGGGCTGTTAGAATATGCCCAGTTCGCTAGTCAAACCTGT
GAGTTCAACATGATAGAGCAGAGTGGACCACCCCATGAACCTCGATTTAA
ATTCCAGGTTGTCATCAATGGCCGAGAGTTTCCCCCAGCTGAAGCTGGAA
GCAAGAAAGTGGCCAAGCAGGATGCAGCTATGAAAGCCATGACAATTCTG
CTAGAGGAAGCCAAAGCCAAGGACAGTGGAAAATCAGAAGAATCATCCCA
CTATTCCACAGAGAAAGAATCAGAGAAGACTGCAGAGTCCCAGACCCCCA
CCCCTTCAGCCACATCCTTCTTTTCTGGGAAGAGCCCCGTCACCACACTG
CTTGAGTGTATGCACAAATTGGGGAACTCCTGCGAATTCCGTCTCCTGTC
CAAAGAAGGCCCTGCCCATGAACCCAAGTTCCAATACTGTGTTGCAGTGG
GAGCCCAAACTTTCCCCAGTGTGAGTGCTCCCAGCAAGAAAGTGGCAAAG
CAGATGGCCGCAGAGGAAGCCATGAAGGCCCTGCATGGGGAGGCGACCAA
CTCCATGGCTTCTGATAACCAGCCTGAAGGTATGATCTCAGAGTCACTTG
ATAACTTGGAATCCATGATGCCCAACAAGGTCAGGAAGATTGGCGAGCTC
GTGAGATACCTGAACACCAACCCTGTGGGTGGCCTTTTGGAGTACGCCCG
CTCCCATGGCTTTGCTGCTGAATTCAAGTTGGTCGACCAGTCCGGACCTC
CTCACGAGCCCAAGTTCGTTTACCAAGCAAAAGTTGGGGGTCGCTGGTTC
CCAGCCGTCTGCGCACACAGCAAGAAGCAAGGCAAGCAGGAAGCAGCAGA
TGCGGCTCTCCGTGTCTTGATTGGGGAGAACGAGAAGGCAGAACGCATGG
GTTTCACAGAGCTCCCTCTCACTGGCAGCACCTTCCATGACCAGATAGCC
ATGCTGAGCCACCGGTGCTTCAACACTCTGACTAACAGCTTCCAGCCCTC
CTTGCTCGGCCGCAAGATTCTGGCCGCCATCATTATGAAAAAAGACTCTG
AGGACATGGGTGTCGTCGTCAGCTTGGGAACAGGGAATCGCTGTGTGAAA
GGAGATTCTCTCAGCCTAAAAGGAGAAACTGTCAATGACTGCCATGCAGA
AATAATCTCCCGGAGAGGCTTCATCAGGTTTCTCTACAGTGAGTTAATGA
AATACAACTCCCAGACTGCGAAGGATAGTATATTTGAACCTGCTAAGGGA
GGAGAAAAGCTCCAAATAAAAAAGACTGTGTCATTCCATCTGTATATCAG
CACTGCTCCGTGTGGAGATGGCGCCCTCTTTGACAAGTCCTGCAGCGACC
GTGCTATGGAAAGCACAGAATCCCGCCACTACCCTGTCTTCGAGAATCCC
AAACAAGGAAAGCTCCGCACCAAGGTGGAGAACGGAGAAGGCACAATCCC
TGTGGAATCCAGTGACATTGTGCCTACGTGGGATGGCATTCGGCTCGGGG
AGAGACTCCGTACCATGTCCTGTAGTGACAAAATCCTACGCTGGAACGTG
CTGGGCCTGCAAGGGGCACTGTTGACCCACTTCCTGCAGCCCATTTATCT
CAAATCTGTCACATTGGGTTACCTTTTCAGCCAAGGGCATCTGACCCGTG
CTATTTGCTGTCGTGTGACAAGAGATGGGAGTGCATTTGAGGATGGACTA
CGACATCCCTTTATTGTCAACCACCCCAAGGTTGGCAGAGTCAGCATATA
TGATTCCAAAAGGCAATCCGGGAAGACTAAGGAGACAAGCGTCAACTGGT
GTCTGGCTGATGGCTATGACCTGGAGATCCTGGACGGTACCAGAGGCACT
GTGGATGGGCCACGGAATGAATTGTCCCGGGTCTCCAAAAAGAACATTTT
TCTTCTATTTAAGAAGCTCTGCTCCTTCCGTTACCGCAGGGATCTACTGA
GACTCTCCTATGGTGAGGCCAAGAAAGCTGCCCGTGACTACGAGACGGCC
AAGAACTACTTCAAAAAAGGCCTGAAGGATATGGGCTATGGGAACTGGAT
TAGCAAACCCCAGGAGGAAAAGAACTTTTATCTCTGCCCAGTATAGTATG
CTCCAGTGACAGATGGATTAGGGTGTGTCATACTAGGGTGTGAGAGAGGT
AGGTCGTAGCATTCCTCATCACATGGTCAGGGGATTTTTTTTTCTCCTTT
TTTTTTCTTTTTAAGCCATAATTGGTGATACTGAAAACTTTGGGTTCCCA
TTTATCCTGCTTTCTTTGGGATTGCTAGGCAAGGTCTGGCCAGGCCCCCC
TTTTTTCCCCCAAGTGAAGAGGCAGAAACCTAAGAAGTTATCTTTTCTTT
CTACCCAAAGCATACATAGTCACTGAGCACCTGCGGTCCATTTCCTCTTA
AAAGTTTTGTTTTGATTTGTTTCCATTTCCTTTCCCTTTGTGTTTGCTAC
ACTGACCTCTTGCGGTCTTGATTAGGTTTCAGTCAACTCTGGATCATGTC
AGGGACTGATAATTTCATTTGTGGATTACGCAGACCCCTCTACTTCCCCT
CTTTCCCTTCTGAGATTCTTTCCTTGTGATCTGAATGTCTCCTTTTCCCC
CTCAGAGGGCAAAGAGGTGAACATAAAGGATTTGGTGAAACATTTGTAAG
GGTAGGAGTTGAAAACTGCAGTTCCCAGTGCCACGGAAGTGTGATTGGAG
CCTGCAGATAATGCCCAGCCATCCTCCCATCCTGCACTTTAGCCAGCTGC
AGGGCGGGCAAGGCAAGGAAAGCTGCTTCCCTGGAAGTGTATCACTTTCT
CCGGCAGCTGGGAAGTCTAGAACCAGCCAGACTGGGTTAAGGGAGCTGCT
CAAGCAATAGCAGAGGTTTCACCCGGCAGGATGACACAGACCACTTCCCA
GGGAGCACGGGCATGCCTTGGAATATTGCCAAGCTTCCAGCTGCCTCTTC
TCCTAAAGCATTCCTAGGAATATTTTCCCCGCCAATGCTGGGCGTACACC
CTAGCCAACGGGACAAATCCTAGAGGGTATAAAATCATCTCTGCTCAGAT
AATCATGACTTAGCAAGAATAAGGGCAAAAAATCCTGTTGGCTTAACGTC
ACTGTTCCACCCGGTGTAATATCTCTCATGACAGTGACACCAAGGGAAGT
TGACTAAGTCACATGTAAATTAGGAGTGTTTTAAAGAATGCCATAGATGT
TGATTCTTAACTGCTACAGATAACCTGTAATTGAGCAGATTTAAAATTCA
GGCATACTTTTCCATTTATCCAAGTGCTTTCATTTTTCCAGATGGCTTCA
GAAGTAGGCTCGTGGGCAGGGCGCAGACCTGATCTTTATAGGGTTGACAT
AGAAAGCAGTAGTTGTGGGTGAAAGGGCAGGTTGTCTTCAAACTCTGTGA
GGTAGAATCCTTTGTCTATACCTCCATGAACATTGACTCGTGTGTTCAGA
GCCTTTGGCCTCTCTGTGGAGTCTGGCTCTCTGGCTCCTGTGCATTCTTT
GAATAGTCACTCGTAAAAACTGTCAGTGCTTGAAACTGTTTCCTTTACTC
ATGTTGAAGGGACTTTGTTGGCTTTTAGAGTGTTGGTCATGACTCCAAGA
GCAGAGCAGGGAAGAGCCCAAGCATAGACTTGGTGCCGTGGTGATGGCTG
CAGTCCAGTTTTGTGATGCTGCTTTTACGTGTCCCTCGATAACAGTCAGC
TAGACACACTCAGGAGGACTACTGAGGCTCTGCGACCTTCAGGAGCTGAG
CCTGCCTCTCTCCTTTAGATGACAGACCTTCATCTGGGAACGTGCTGAGC
CAGCACCCTCAGATGATTTCCCTCCAAACTGCTGACTAGGTCATCCTCTG
TCTGGTAGAGACATTCACATCTTTGCTTTTATTCTATGCTCTCTGTACTT
TTGACCAAAAATTGACCAAAGTAAGAAAATGCAAGTTCTAAAAATAGACT
AAGGATGCCTTTGCAGAACACCAAAGCATCCCAAGGAACTGGTAGGGAAG
TGGCGCCTGTCTCCTGGAGTGGAAGAGGCCTGCTCCCTGGCTCTGGGTCT
GCTGGGGGCACAGTAAATCAGTCTTGGCACCCACATCCAGGGCAGAGAGG
TCTGTGGTTCTCAGCATCAGAAGGCAGCGCAGCCCCTCTCCTCTTCAGGC
TACAGGGTTGTCACCTGCTGAGTCCTCAGGTTGTTTGGCCTCTCTGGTCC
ATCTTGGGCATTAGGTTCTCCAGCAGAGCTCTGGCCAGCTGCCTCTTCTT
TAACTGGGAACACAGGCTCTCACAAGATCAGAACCCCCACTCACCCCCAA
GATCTTATCTAGCAAGCCTGTAGTATTCAGTTTCTGTTGTAGGAAGAGAG
CGAGGCATCCCTGAATTCCACGCATCTGCTGGAAACGAGCCGTGTCAGAT
CGCACATCCCTGCGCCCCCATGCCCCTCTGAGTCACACAGGACAGAGGAG
GCAGAGCTTCTGCCCACTGTTATCTTCACTTTCTTTGTCCAGTCTTTTGT
TTTTAATAAGCAGTGACCCTCCCTACTCTTCTTTTTAATGATTTTTGTAG
TTGATTTGTCTGAACTGTGGCTACTGTGCATTCCTTGAATAATCACTTGT
AAAAATTGTCAGTGCTTGAAGCTGTTTCCTTTACTCACATTGAAGGGACT
TCGTTGGTTTTTTGGAGTCTTGGTTGTGACTCCAAGAGCAGAGTGAGGAA
GACCCCCAAGCATAGACTCGGGTACTGTGATGATGGCTGCAGTCCAGTTT
TATGATTCTGCTTTTATGTGTCCCTTGATAACAGTGACTTAACAATATAC
ATTCCTCATAAATAAAAAAAAAACAAGAATCTGAATTCTTAGAAA

SEQ ID NO: 25 - Human ADAR1 - Transcript Variant 10 coding Sequence

ATGGCCGAGATCAAGGAGAAAATCTGCGACTATCTCTTCAATGTGTCTGA
CTCCTCTGCCCTGAATTTGGCTAAAAATATTGGCCTTACCAAGGCCCGAG
ATATAAATGCTGTGCTAATTGACATGGAAAGGCAGGGGGATGTCTATAGA
CAAGGGACAACCCCTCCCATATGGCATTTGACAGACAAGAAGCGAGAGAG
GATGCAAATCAAGAGAAATACGAACAGTGTTCCTGAAACCGCTCCAGCTG
CAATCCCTGAGACCAAAAGAAACGCAGAGTTCCTCACCTGTAATATACCC
ACATCAAATGCCTCAAATAACATGGTAACCACAGAAAAAGTGGAGAATGG
GCAGGAACCTGTCATAAAGTTAGAAAACAGGCAAGAGGCCAGACCAGAAC
CAGCAAGACTGAAACCACCTGTTCATTACAATGGCCCCTCAAAAGCAGGG
TATGTTGACTTTGAAAATGGCCAGTGGGCCACAGATGACATCCCAGATGA
CTTGAATAGTATCCGCGCAGCACCAGGTGAGTTTCGAGCCATCATGGAGA
TGCCCTCCTTCTACAGTCATGGCTTGCCACGGTGTTCACCCTACAAGAAA
CTGACAGAGTGCCAGCTGAAGAACCCCATCAGCGGGCTGTTAGAATATGC
CCAGTTCGCTAGTCAAACCTGTGAGTTCAACATGATAGAGCAGAGTGGAC
CACCCCATGAACCTCGATTTAAATTCCAGGTTGTCATCAATGGCCGAGAG
TTTCCCCCAGCTGAAGCTGGAAGCAAGAAAGTGGCCAAGCAGGATGCAGC
TATGAAAGCCATGACAATTCTGCTAGAGGAAGCCAAAGCCAAGGACAGTG
GAAAATCAGAAGAATCATCCCACTATTCCACAGAGAAAGAATCAGAGAAG
ACTGCAGAGTCCCAGACCCCCACCCCTTCAGCCACATCCTTCTTTTCTGG
GAAGAGCCCCGTCACCACACTGCTTGAGTGTATGCACAAATTGGGGAACT
CCTGCGAATTCCGTCTCCTGTCCAAAGAAGGCCCTGCCCATGAACCCAAG
TTCCAATACTGTGTTGCAGTGGGAGCCCAAACTTTCCCCAGTGTGAGTGC
TCCCAGCAAGAAAGTGGCAAAGCAGATGGCCGCAGAGGAAGCCATGAAGG
CCCTGCATGGGGAGGCGACCAACTCCATGGCTTCTGATAACCAGCCTGAA
GGTATGATCTCAGAGTCACTTGATAACTTGGAATCCATGATGCCCAACAA
GGTCAGGAAGATTGGCGAGCTCGTGAGATACCTGAACACCAACCCTGTGG
GTGGCCTTTTGGAGTACGCCCGCTCCCATGGCTTTGCTGCTGAATTCAAG
TTGGTCGACCAGTCCGGACCTCCTCACGAGCCCAAGTTCGTTTACCAAGC
AAAAGTTGGGGGTCGCTGGTTCCCAGCCGTCTGCGCACACAGCAAGAAGC
AAGGCAAGCAGGAAGCAGCAGATGCGGCTCTCCGTGTCTTGATTGGGGAG
AACGAGAAGGCAGAACGCATGGGTTTCACAGAGCTCCCTCTCACTGGCAG
CACCTTCCATGACCAGATAGCCATGCTGAGCCACCGGTGCTTCAACACTC
TGACTAACAGCTTCCAGCCCTCCTTGCTCGGCCGCAAGATTCTGGCCGCC
ATCATTATGAAAAAAGACTCTGAGGACATGGGTGTCGTCGTCAGCTTGGG
AACAGGGAATCGCTGTGTGAAAGGAGATTCTCTCAGCCTAAAAGGAGAAA
CTGTCAATGACTGCCATGCAGAAATAATCTCCCGGAGAGGCTTCATCAGG
TTTCTCTACAGTGAGTTAATGAAATACAACTCCCAGACTGCGAAGGATAG
TATATTTGAACCTGCTAAGGGAGGAGAAAAGCTCCAAATAAAAAAGACTG
TGTCATTCCATCTGTATATCAGCACTGCTCCGTGTGGAGATGGCGCCCTC
TTTGACAAGTCCTGCAGCGACCGTGCTATGGAAAGCACAGAATCCCGCCA
CTACCCTGTCTTCGAGAATCCCAAACAAGGAAAGCTCCGCACCAAGGTGG
AGAACGGAGAAGGCACAATCCCTGTGGAATCCAGTGACATTGTGCCTACG
TGGGATGGCATTCGGCTCGGGGAGAGACTCCGTACCATGTCCTGTAGTGA
CAAAATCCTACGCTGGAACGTGCTGGGCCTGCAAGGGGCACTGTTGACCC
ACTTCCTGCAGCCCATTTATCTCAAATCTGTCACATTGGGTTACCTTTTC
AGCCAAGGGCATCTGACCCGTGCTATTTGCTGTCGTGTGACAAGAGATGG
GAGTGCATTTGAGGATGGACTACGACATCCCTTTATTGTCAACCACCCCA
AGGTTGGCAGAGTCAGCATATATGATTCCAAAAGGCAATCCGGGAAGACT
AAGGAGACAAGCGTCAACTGGTGTCTGGCTGATGGCTATGACCTGGAGAT
CCTGGACGGTACCAGAGGCACTGTGGATGGGCCACGGAATGAATTGTCCC
GGGTCTCCAAAAAGAACATTTTTCTTCTATTTAAGAAGCTCTGCTCCTTC
CGTTACCGCAGGGATCTACTGAGACTCTCCTATGGTGAGGCCAAGAAAGC
TGCCCGTGACTACGAGACGGCCAAGAACTACTTCAAAAAAGGCCTGAAGG
ATATGGGCTATGGGAACTGGATTAGCAAACCCCAGGAGGAAAAGAACTTT
TATCTCTGCCCAGTATAG

SEQ ID NO: 26 - Human ADAR1 - Transcript Variant 10 Amino Acid Sequence (aka isoform-f)

MAEIKEKICDYLFNVSDSSALNLAKNIGLTKARDINAVLIDMERQGDVYR
QGTTPPIWHLTDKKRERMQIKRNTNSVPETAPAAIPETKRNAEFLTCNIP
TSNASNNMVTTEKVENGQEPVIKLENRQEARPEPARLKPPVHYNGPSKAG
YVDFENGQWATDDIPDDLNSIRAAPGEFRAIMEMPSFYSHGLPRCSPYKK
LTECQLKNPISGLLEYAQFASQTCEFNMIEQSGPPHEPRFKFQVVINGRE
FPPAEAGSKKVAKQDAAMKAMTILLEEAKAKDSGKSEESSHYSTEKESEK
TAESQTPTPSATSFFSGKSPVTTLLECMHKLGNSCEFRLLSKEGPAHEPK
FQYCVAVGAQTFPSVSAPSKKVAKQMAAEEAMKALHGEATNSMASDNQPE
GMISESLDNLESMMPNKVRKIGELVRYLNTNPVGGLLEYARSHGFAAEFK
LVDQSGPPHEPKFVYQAKVGGRWFPAVCAHSKKQGKQEAADAALRVLIGE
NEKAERMGFTELPLTGSTFHDQIAMLSHRCFNTLTNSFQPSLLGRKILAA
IIMKKDSEDMGVVVSLGTGNRCVKGDSLSLKGETVNDCHAEIISRRGFIR
FLYSELMKYNSQTAKDSIFEPAKGGEKLQIKKTVSFHLYISTAPCGDGAL
FDKSCSDRAMESTESRHYPVFENPKQGKLRTKVENGEGTIPVESSDIVPT
WDGIRLGERLRTMSCSDKILRWNVLGLQGALLTHFLQPIYLKSVTLGYLF
SQGHLTRAICCRVTRDGSAFEDGLRHPFIVNHPKVGRVSIYDSKRQSGKT
KETSVNWCLADGYDLEILDGTRGTVDGPRNELSRVSKKNIFLLFKKLCSF
RYRRDLLRLSYGEAKKAARDYETAKNYFKKGLKDMGYGNWISKPQEEKNF
YLCPV

In some embodiments, an ADAR1 polypeptide or a characteristic portion thereof, e.g., those included and/or expressed in engineered cells, tissues, non-human animals, etc., is a polypeptide comprising one or more characteristic sequence elements or portions of a human ADAR1. In some embodiments, an ADAR1 polypeptide or a characteristic portion thereof, e.g., those included and/or expressed in engineered cells, tissues, non-human animals, etc., is a polypeptide comprising one or more characteristic sequence elements or portions of a human ADAR1 p110. In some embodiments, an ADAR1 polypeptide or a characteristic portion thereof, e.g., those included and/or expressed in engineered cells, tissues, non-human animals, etc., is a polypeptide comprising one or more characteristic sequence elements or portions of a human ADAR1 p150. In some embodiments, an ADAR1 polypeptide is or comprises an amino acid sequence that is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with exemplary human ADAR1 p110 double stranded RNA binding domain (dsRBD) amino acid sequence of SEQ ID NOs: 27-32. In some embodiments, an ADAR1 polypeptide is or comprises an amino acid sequence that is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with exemplary human ADAR1 p110 Z-DNA binding domain amino acid sequence of SEQ ID NOs: 33-37. In some embodiments, an ADAR1 polypeptide is or comprises an amino acid sequence that is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with exemplary human ADAR1 p110 Deaminase domain amino acid sequence of SEQ ID NOs: 38-40. In some embodiments, for two or more or each amino acid sequence of SEQ ID NO: 27-40, an ADAR1 polypeptide independently contains an amino acid sequence that is the same, or differs by no more than 1-10 (e.g., 1-8, 1-5, 1, 2, 3, 4, 5, 6, 7, 8, 9, or 10) amino acids, or has about or at least about 90%-100% (e.g., 90%, 91%, 92%, 93%, 94%, 95%, 96%, 97%, 98%, or 99%) homology.

In certain embodiments, an ADAR1 polypeptide or a characteristic portion thereof, e.g., included in an engineered cell, tissue or non-human animal (e.g., a rodent, e.g., a rat or mouse) is represented by, is or comprises one or more ADAR1 double stranded RNA binding domains (dsRBD) or a characteristic portion thereof. In certain embodiments, an ADAR1 dsRBD amino acid sequence is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with, the amino acid sequence of SEQ ID NOs: 27-32.

SEQ ID NO: 27 - Human ADAR1 - an Amino Acid Sequence

PIQVLHEYGMKTKNIPVYECERSDVQIHVPTFTFRVTVGDITCTGEGTSK
KLAKHRAAEAAINILK

SEQ ID NO: 28 - Human ADAR1 - an Amino Acid Sequence

NPISGLLEYAQFASQTCEFNMIEQSGPPHEPRFKFQVVINGREFPPAEAG
SKKVAKQDAAMKAMTILLE

SEQ ID NO: 29 - Human ADAR1 - an Amino Acid Sequence

SPVTTLLECMHKLGNSCEFRLLSKEGPAHEPKFQYCVAVGAQTFPSVSAP
SKKVAKQMAAEEAMKALHG

SEQ ID NO: 30 - Human ADAR1 - an Amino Acid Sequence

NPVGGLLEYARSHGFAAEFKLVDQSGPPHEPKFVYQAKVGGRWFPAVCAH
SKKQGKQEAADAALRVLIG

SEQ ID NO: 31 - ADAR1 - an Amino Acid Sequence

PKSLLQELAQKRKLPPEYELVKEEGPDHAPRFTVTVKVGGRTGEGEGSSK
KEAKQRAAEAALRSLK

SEQ ID NO: 32 - ADAR1 - an Amino Acid Sequence

NPV-GLLEYA------CEF-L--QSGPPHEPKF-YQV-VGGR--PPV-A-
SKKBAKQ-AA--AM--L-G

In certain embodiments, an ADAR1 polypeptide or a characteristic portion thereof, e.g., included in an engineered cell, tissue or non-human animal (e.g., a rodent, e.g., a rat or mouse) is represented by, is or comprises one or more ADAR1 Z-DNA binding domain or a characteristic portion thereof. In certain embodiments, an ADAR1 Z-DNA binding domain amino acid sequence is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with, the amino acid sequence of SEQ ID NOs: 33-37.

SEQ ID NO: 33 - Human ADAR1 - an Amino Acid Sequence

DMAEIKEKICDYLFNVSDSSALNLAKNIGLTKARDINAVLIDMERQGDVY
RQGTTPPIWHLTDKK

SEQ ID NO: 34 - Human ADAR1 - an Amino Acid Sequence

IYQDQEQRILKFLEELGGKATTAHDLSGKLGTKKEINRVLYSLAKKGKLQ
KEAGTPPLWKIAVST

SEQ ID NO: 35 - Human ADAR1 - an Amino Acid Sequence

LSIYQDQEQRILKFLEELGEGKATTAHDLSGKLGTPKKEINRVLYSLAKK
GKLQKEAGTPPLWKIAV

SEQ ID NO: 36 - Human ADAR1 - an Amino Acid Sequence

FLDMAEIKEKICDYLFNVSDSSALNLAKNIGLTKARDINAVLIDMERQGD
VYRQGTTPPIWHLTD

SEQ ID NO: 37 - ADAR1 - an Amino Acid Sequence

DAQECAEKILELLENLGEGKAATAIALERGLSTKREINRVLYDLERKGDV
YREDGTPPRWFLTCAK

In certain embodiments, an ADAR1 polypeptide or a characteristic portion thereof, e.g., included in an engineered cell, tissue or non-human animal (e.g., a rodent, e.g., a rat or mouse) is represented by, is or comprises one or more ADAR1 deaminase domains or a characteristic portion thereof. In certain embodiments, an ADAR1 deaminase domain amino acid sequence is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with, the amino acid sequence of SEQ ID NOs: 38-40.

SEQ ID NO: 38 - Human ADAR1 - an Amino Acid Sequence

SLGTGNRCVKGDSLSLKGETVNDCHAEIISRRGFIRFLYSELMKYNSQTA
KDSIFEPAKGGEKLQIKKTVSFHLYISTAPCGDGALFDKSCSDRAMESTE
SRHYPVFENPKQGKLRTKVENGEGTIPVESSDIVPTWDGIRLGERLRTMS
CSDKILRWNVLGLQGALLTHFLQPIYLKSVTLGYLFSQGHLTRAICCRVT
RDGSAFEDGLRHPFIVNHPKVGRVSIYDSKRQSGKTKETSVNWCLADGYD
LEILDGTRGTVDGPRNELSRVSKKNIFLLFKKLCSFRYRRDLLRLSYGEA
KKAARDYETAKNYFKKGLKDMGYGNWISKPQEEKNF

SEQ ID NO: 39 - Human ADAR1 - an Amino Acid Sequence

SHRCFTLTKILAAIIMKKMGVVVSLGTGNRCVKDSLSLKGETVNDCHAEI
ISRRGFIRFLYSELMKSIFEPAKGGLQIKKTVSFHLYISTAPCGDGALFD
KSCKLRTKVENGEGTIPDIVPTWDGIRERLRTMSCSDKILRWNVLGLQGA
LLTHFLQPIYLKSVTLGYLFSQGHLTRAICCRVTRDGLRHPFIVNHPKSV
NWCLADGLEILDGTRGTVPRNELSRVSKKNIFLLFKKLCSFRYRR.LSYG
EAKKAARDYETAKKGLKDMGYGNWIPQEEKNFY

SEQ ID NO: 40 - ADAR1 - an Amino Acid Sequence

VLEKFSLPTILAGVVMTNEKQVVSLGTGTKCISEKLSPNGLVLNDCHAEI
LARRGFLRFLYSELQLSIFEKNKEGYKLKSNVLFHLYISTLPCGDASIFS
PLEKLRTKIEIGEGTVPDIVQTWDGIGERLLSMSCSDKIARWNVLGVQGA
LLSHFIEPIYLSSIVLGSLYSAEHLERALYGRLDPDGLPTPFRVNRPLSV
NWSQGDELEILNGLTGKTSLGSPSRLCKKALFRLFQKLCSKLKRDISYAE
AKEAASEYQEAKEALNKAGLGSWIPPEQDQFK

In certain embodiments, an ADAR1 polynucleotide or an ADAR1 gene incorporated into a non-human animal (e.g., a rodent, e.g., a rat or mouse) is represented by or comprises a sequence encoding a non-human primate (NHP) ADAR1 transcript or a characteristic portion thereof. In some embodiments, an ADAR1 polynucleotide or a NHP ADAR1 transcript is transcript variant X1. In certain embodiments, ADAR1 polynucleotide or a NHP ADAR1 gene comprises, or consists of, a nucleotide sequence that is the same as, differs by no more than 1, 2, 3, 4, 5, 6, 7, 8, 9, or 10 nucleotides from, or has at least has at least 90%, 92%, 94%, 96%, 98%, or 99% homology with, the nucleotide sequence of SEQ ID NO: 41 or SEQ ID NO: 42 or a characteristic portion thereof. In some embodiments, an ADAR1 polynucleotide or a NHP ADAR1 gene comprises, or consists of, a nucleotide sequence that is the same as the nucleotide sequence of SEQ ID NO: 41 or SEQ ID NO: 42 or a characteristic portion thereof. In certain embodiments, an encoded ADAR1 Amino Acid sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the Amino Acid sequence of SEQ ID NO: 43 or a characteristic portion thereof. In certain embodiments, the amino acid sequence of an encoded and/or expressed ADAR1 polypeptide or a characteristic portion thereof, is or comprises a sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the amino acid sequence of SEQ ID NO: 43 or a characteristic portion thereof.

SEQ ID NO: 41 - NHP ADAR1 - Transcript Variant X1 cDNA Sequence

ATGATGTCACCAATCTGCGACCACACCATTGATTCCCGACTGAAGGTAGA
GAGGGCTACGTGGTGGGGGAGGGCGGGGGGAGGGTATCGGCCGCACTGGC
AGCCTCCGGGTGTTCGGCCGTGTCCGGAGGAAGTACAAGACCCGGGGTAT
TCCCTCAACGGATACTACCCCCATCCATTTCAAGGCTATGAGCACAGAAA
GCTCAGATACCAGCAGCCTGGGCCAGGATCTTCCCCCAATAGTTTCCTGC
TTAAGCAAATAGAGTTTCTCAAGGGGCAGATCCCAGAAGCGCCGGTGATT
GGAAAGCAGACACCATCACTGCCACCTTCCCTCCCAGGACTCCAGCCAAG
GTTTCCAGTACTACCTGCCTCTAGTACCAGAGGCAGGCAAGTGGATATCA
GGGGTGTCCCCAGGTGCGTGCATCTCGGAAGTCAGGGGCTCCAGAGAGGG
TTCCAGCATCCTTCACCACGTGGCAGGATTCCGCCACGGAGAGGTGTTGA
TTGCCTTTCCTCACAATTCCAGGAACTGAGTATCAACCAAGATCAGGAAC
AAAGGATCTTAAAGTTCTTGGAAGAGCTTGGGGAAGGGAAGGCCACTACA
GCACATGATCTGTCTTGGAAACTGGGGGCTCCAAAGAAAGAAATCAATCG
AGTTTTATACTCCCTGGCAAAGAAGGGCAAGCTACAGAAAGAGGCAGGAA
CACCCCCTTTGTGGAAAATCGCGGTCTCCACTCAGGCTTGGAACCAGCAC
AGCAGAGTGGTAAGACCAGACGGTCATAGCCAAGGAGCCCCAAACTCAGA
CCCAAATTTGGAACCTGAAGACAGAAACTCCACATCTGTCTCAGAAGATC
TTGAGCCTTTTATCTCAGTCTCAGCTCAGGCTTGGAACCAGCACAGCGGA
GTGGTAAGACCAGACGGTCATAGCCAAGGAGCCCCAAACTCAGAGTCGAG
TTTGGAACCTGAAGACAGAAACTCCACATCTGCCTTGGAAGATCCTCTTG
AGTTTTTTGACATGGCTGAGATCAAGGAGAAAATCTGCGACTATCTCTTC
AATGTGTCTGACTCCTCTGCCCTGAATTTGGCTAAAAATATTGGCCTCAC
CAAGGCCCGAGATATAAATGCCGTGCTAATTGACATGGAAAGGCAGGGGG
ATGTCTATAGACAAGGGACAACCCCTCCCATATGGCATTTGACAGACAAG
AAGCGAGAGAGGATGCAAATCAAGAGAAATACGAACAGTGTTCCTGAAAC
CGCTCCAGCTGCAATCCCTGAGACCAAAAGAAACACAGAGTTCCTCACCT
GTAATATACCCACATCAAATGCCTCAAATAACATAGTAACCACCGAAAAA
GTGGAGAATGGGCAGGAACCTGTCATAAAGTTAGAAAACAGTCAGGAGGC
AAGACCAGAACCAGTAAGACTGAAACCACCTGTTCATAACAATGGCCCCT
CAAAAGCAGGGTATGTTGACTTTGAAAATGGCCAGTGGGCCACAGATGAC
ATCCCAGATGACTTGAATAGTATCCGCGCAGCACCAGGTGAGTTTCGAGC
CATCATGGAGATGCCCTCCTTCTACAGTCATGGCTTGCCACGGTGTTCAC
CCTACAAGAAACTGACAGAGTGCCAGCTGAAGAACCCCATCAGCGGGCTG
TTAGAATATGCCCAGTTCGCTAGTCAAACCTGTGAGTTCAACATGATAGA
GCAGAGTGGACCACCCCATGAACCTCGATTTAAATTCCAGGTTGTCATCA
ATGGCCGAGAGTTTCCCCCAGCTGAAGCTGGAAGCAAGAAAGTGGCCAAG
CAGGATGCAGCTATGAAAGCCATGACAATTCTGCTAGAGGAAGCCAAAGC
CAAGGACAGTGGAAAATCAGAAGAATCATCCCACTATTCCACAGAAAAAG
AATCAGAGAAGACCACAGAGTCCCAGACCCCCACCCCTTCAGCTACATCC
TTCTTTTCTGGGAAGAGCCCCGTCACCACACTGCTCGAGTGTATGCACAA
ATTGGGGAACTCCTGCGAATTCCGTCTCCTGTCCAAAGAAGGCCCTGCCC
ATGAGCCCAAGTTCCAGTACTGTGTTGCAGTGGGAGCCCAAACTTTCCCC
AGTGTGAGTGCCCCCAGCAAGAAAGTGGCAAAGCAGATGGCCGCAGAGGA
AGCCATGAAGGCCCTGCATGGGGAGGCAACCAACTCCATGACTTCTGATG
ACCAGCCCGAAGGTATGATCTCAGAGTCACTTGATAACTTGGAATCCGTG
ATGCCCAACAAGGTCAGGAAGATTGGCGAGCTCGTGAGATACTTGAACAC
CAATCCTGTGGGTGGCCTTTTGGAGTACGCCCGCTCCCATGGCTTTGCTG
CTGAATTCAAGTTGGTCGACCAGTCTGGACCTCCTCACGAGCCCAAGTTC
GTTTACCAAGCAAAAGTTGGGGGTCGCTGGTTCCCAGCCGTCTGCGCACA
CAGCAAGAAGCAAGGCAAGCAGGAAGCAGCAGATGCGGCTCTCCGTGTCT
TGATTGGGGAGAACGAGAAGGCAGAACGCATGGGTTTCACAGAGGTAACC
CCAGTGACAGGGGCCAGTCTCAGAAGAACTATGCTCCTCCTCTCAAGGTC
CCCAGAAGCACAGCCAAAGACACTCCCTCTCACTGGCAGCACCTTCCATG
ACCAGATAGCCATGCTGAGCCACCGGTGCTTCAACACTCTGACTAACAGC
TTCCAGCCTTCCTTGCTCGGCCGCAAGATTCTGGCCGCCATCGTTATGAA
AAAAGACTCTGAGGACATGGGTGTCGTCGTCAGCTTGGGAACAGGGAATC
GCTGTGTGAAAGGAGATTCTCTCAGCCTGAAGGGAGAAACTGTCAATGAC
TGCCATGCAGAAATCATCTCCCGGAGAGGCTTCATCAGGTTTCTCTACAG
TGAGTTAATGAAATACAACCCCCAGACTGCGAAGGATAGTATATTTGAAC
CTGCTAAGGGAGGAGAAAAGCTCCAAATAAAAAAGACTGTGTCATTCCAT
CTCTATATCAGCACCGCTCCATGTGGAGATGGTGCCCTCTTTGACAAGTC
CTGCAGCGACCGTGCTATGGAAAGCACAGACTCCCGCCACTACCCTGTCT
TTGAGAATCCCAAACAAGGCAAGCTCCGCACCAAGGTGGAAAACGGAGAA
GGCACAATCCCTGTGGAATCCAGTGACATTGTACCTACGTGGGATGGCAT
TCGGCTCGGGGAGAGACTCCGTACCATGTCCTGTAGTGACAAAATCCTAC
GCTGGAATGTGCTGGGCCTGCAAGGGGCACTGTTGACCCACTTCCTGCAG
CCCATTTATCTCAAATCTGTTACATTGGGTTACCTTTTCAGCCAAGGGCA
TCTGACCCGGGCTATTTGCTGTCGTGTGACAAGAGATGGGAGTGCATTTG
AGGATGGACTACGACATCCCTTTATTGTCAACCACCCCAAGGTTGGCAGA
GTCAGCGTATATGATTCCAAAAGGCAATCTGGGAAGACTAAGGAGACAAG
CGTCAACTGGTGTCTGGCTGATGGCTATGACCTGGAGATCCTGGATGGTA
CCAGAGGCACCGTGGATGGGCCACGGAATGAATTGTCCCGGGTCTCCAAA
AAGAACATTTTTCTTCTATTTAAGAAGCTCTGCTCCTTCCGTTACCGCAG
GGATCTACTGAGACTCTCCTATGGTGAGGCCAAGAAAGCTGCCCGTGACT
ACGAGACGGCCAAGAACTACTTCAAAAAAAGCTTGAAGGATATGGGCTAT
GGGAACTGGATTAGCAAACCCCAGGAGGAAAAGAACTTTTATCTCTGCCC
AGTATAGTATGCTCCGGTGACAGATGGATTAGGGTGTGTCATACTAGGGT
GAGAGAGAGGTAGGTCGTAGCGTTCCTCATCACATGGTCAGGGGATTTTT
TTTTCCTCCTTTTTTTTCTTTTTAAGCCATAATTGGTGATACTGAAAACT
TTGGGTTCCCATTTATCCTGCTTTCTTTGGGATTGCTAGGCAAGGTCTGG
CCAGGCCCCCCTTTTTTTCCCCCAAGTGAAGAGGTAGAAACCTAAGAAGT
TATCTTTTCTTTCTATAACTCAAAGCATACATAGTCACTGAGCACCTGCA
GTCCATTTCCTCTTAAAAGTTTTTTTTTTTTTCCATTTCCTTTCCCTTTG
TGTTTGCTACACTGACCTCTTGTGGTCTTGATTAGGTTTCAGTCAATTCT
GGATCATGTCCGAGACTGATGATTTCTTTTGTGGATTACGCAGACCCCTC
TACTTCCCCCTTCCCCTTCTGAGGTTCTTTCCTTGTGATCAGAATTTCTC
CTTTTCTCCCTCAGAGGGTAAAAAGGTGAACTTAAAGGATCTGGTGAAAC
ATTTGTAAGGGTAGGAGTTGAAAACTGCAGTTCCCAGTGTCACGGAAGTG
TGATTGGAGCCTACAGATAATGCCCTGCCATCCTCCCATCCTGCACTTTA
GCCAGCTGCAGGGCGGGCAAGGCAAGGAAAGCTGCTTCCCTGGAAGTGTG
CCCCTTTCCCCGGCAGCAGGGAAGTCTAGAACCAGCCAGACTGGGTTAAG
GGAGTTGCCCAAGCCATAGCGGAGGTTTCACCCAGCAAGGTGACACAGAC
CACTTCCCAGGAGCACGGGCATGCCTTGGGATATTGCCAAGCTTCCAGCT
GCCTCTTCTCCTAAAGCACTCCTAGGAATTTGCCCTGCAAATGCTGGGCG
TGCACCCTAGCCAACGTAGGAAAAAATCCAGGACAAATCCTAGAGGGTAG
AAAATCATCTCTGCTCAGATAATCATGACTTAGCAAGAATAAGGGCAAAA
AATCCTGTTGGCTCACGTCACTGTTCTACCCTGCATAATATCTCTCATGA
CAGTGACACCAAGAGAAGTTGACTAAGTCATATGTAAATTAGGAGTGTTT
AAAAGAATGCCATAGATGTTGATTCTTAACTGCTACAGACAACCTATAAT
TGAGCAGATTTAAAATTCAGGCATACTTTTCCATTTATCCAAGTGCTTTC
ATTTTTCCAGATGGCTTCAGAAGTAGGCTCGTGGGCAGAGCGCAGACCTG
ATCTTTATAGTGTTGACATAGAAAGCAGTAGTAGTGGGTGAAAGGACAGG
TTGTCTTCAAACTCTGTGAGGTGGAATCTTTTGTCTACACCTCCATGAAC
ATTGACTCGTGTGTTCAGAGCCTTTGGCCTCCCTGTGGAGTCTGGCTCTC
TGGCTCCTGTGCATTCTTTGAATAGTCACTCGTAAAAATTGTCAATGCTT
GAAACTATTTCTTTTACTCATGTTGAAGGGACTTTGTTGACTTTTAGAGT
GTTGGTCATGACTCCAAGAGCAGAGCAGGGAAGAGCCCAAGCATAGACTT
GAGTGCCGTGATGGCTGCGGTCCAGTTTTGTGATTCTGCTTTTACGTGTT
ACTTGGTAACAGTCAGCTAGACACACTCAGGAGGACTACTGAGGCTCTGC
GACCTTCAGGACCTGAGCCTGCCTCTCTCCTTTAGATGACAGACCTTCAT
CTGAGAATGTGCTGAGCCAGCACCCTCAGATGATTTCCCTCCAAACTGCT
GACTAGGTCACCCTCTGTCTGGTAGAGACATTGACATCTTTGCTTTTATT
CTATACCCTCGGTACTTTTGACCAAAAAATGACCAAAATAAGAAAATGCA
ACTTTAAAAAATAGACTAAGGATGCCTTTGCAGAACACCAAAGCATCCCA
AGGAACCGGTAGGGAAATGATGCCTGTCTCCTGGGGTAGAAGAGGCCTGC
TCCCTGGCTCTGGGTCTGCTGGGGGCACAGTAAATCAGTCTTGGCACCCA
CATCCAGGTCAGAGAGGTCTGTGGTTCTCAGCATCAGAAGGCAGCGCAGC
CCCTCTCCAGGCTACAGGGTTGTCACCTGCTGAGTCCTCAAGCTGTTTGG
CCTCTCTGGTCCGTCTTGGGCATTAGATTCTCCAGCAGAGCTCTGGCCAG
CTGCCTCCTCTTTAACTGGGAACACGGGCTCTCACAAGATCAGAACCCCC
ACTCACCCCTGAGATCTTATCTAGCAAGCCTGTAGTATTCCGTTTCTGTT
GTAGGAAGAGCGCGAGGCATCCCTGAATTCCATGCATCTGCTGGAAATGA
GCCGTGTCAGATCGCACACCCCCGCGCCCCCATGCCCCTCTGAGTCACAC
GGGACAGAGGAGGCAGAGCTTCCGCCCACTGTTATCTTCAATTTCTTTGT
CCAGTCTTTTGTTTTTAATAAGCAGTGCCCCTCCCCACTCTTCTTTTTAA
TGATTTTTGTAGTTGATTTGTCTGAACTGTGGCTACTGTGCATTCCTTGA
ATAATCACTTGTAAAAATTGTCAGTGCTTGAAGCTGTTTCCTTTACTCAC
ATTGAAGGGACTTTGTTGGTTTTTTGGAGTCTTGGTTTTGACTCCAAGAA
CAGAGTCAGGAAGACCCCAAGCATAGACTTGGGTACCGTGAAAATGGCTG
CAGTCCAGTTTTATGATTCCGCTTTTATGTGTCCCTTGATAACAGTGACT
TAACAATATACATTCCTCATAAATAAAAAAAAAACCAAGAATCTGAATTC
TTA

SEQ ID NO: 42 - NHP ADAR1 - Transcript Variant X1 Coding Sequence

ATGATGTCACCAATCTGCGACCACACCATTGATTCCCGACTGAAGGTAGA
GAGGGCTACGTGGTGGGGGAGGGCGGGGGGAGGGTATCGGCCGCACTGGC
AGCCTCCGGGTGTTCGGCCGTGTCCGGAGGAAGTACAAGACCCGGGGTAT
TCCCTCAACGGATACTACCCCCATCCATTTCAAGGCTATGAGCACAGAAA
GCTCAGATACCAGCAGCCTGGGCCAGGATCTTCCCCCAATAGTTTCCTGC
TTAAGCAAATAGAGTTTCTCAAGGGGCAGATCCCAGAAGCGCCGGTGATT
GGAAAGCAGACACCATCACTGCCACCTTCCCTCCCAGGACTCCAGCCAAG
GTTTCCAGTACTACCTGCCTCTAGTACCAGAGGCAGGCAAGTGGATATCA
GGGGTGTCCCCAGGTGCGTGCATCTCGGAAGTCAGGGGCTCCAGAGAGGG
TTCCAGCATCCTTCACCACGTGGCAGGATTCCGCCACGGAGAGGTGTTGA
TTGCCTTTCCTCACAATTCCAGGAACTGAGTATCAACCAAGATCAGGAAC
AAAGGATCTTAAAGTTCTTGGAAGAGCTTGGGGAAGGGAAGGCCACTACA
GCACATGATCTGTCTTGGAAACTGGGGGCTCCAAAGAAAGAAATCAATCG
AGTTTTATACTCCCTGGCAAAGAAGGGCAAGCTACAGAAAGAGGCAGGAA
CACCCCCTTTGTGGAAAATCGCGGTCTCCACTCAGGCTTGGAACCAGCAC
AGCAGAGTGGTAAGACCAGACGGTCATAGCCAAGGAGCCCCAAACTCAGA
CCCAAATTTGGAACCTGAAGACAGAAACTCCACATCTGTCTCAGAAGATC
TTGAGCCTTTTATCTCAGTCTCAGCTCAGGCTTGGAACCAGCACAGCGGA
GTGGTAAGACCAGACGGTCATAGCCAAGGAGCCCCAAACTCAGAGTCGAG
TTTGGAACCTGAAGACAGAAACTCCACATCTGCCTTGGAAGATCCTCTTG
AGTTTTTTGACATGGCTGAGATCAAGGAGAAAATCTGCGACTATCTCTTC
AATGTGTCTGACTCCTCTGCCCTGAATTTGGCTAAAAATATTGGCCTCAC
CAAGGCCCGAGATATAAATGCCGTGCTAATTGACATGGAAAGGCAGGGGG
ATGTCTATAGACAAGGGACAACCCCTCCCATATGGCATTTGACAGACAAG
AAGCGAGAGAGGATGCAAATCAAGAGAAATACGAACAGTGTTCCTGAAAC
CGCTCCAGCTGCAATCCCTGAGACCAAAAGAAACACAGAGTTCCTCACCT
GTAATATACCCACATCAAATGCCTCAAATAACATAGTAACCACCGAAAAA
GTGGAGAATGGGCAGGAACCTGTCATAAAGTTAGAAAACAGTCAGGAGGC
AAGACCAGAACCAGTAAGACTGAAACCACCTGTTCATAACAATGGCCCCT
CAAAAGCAGGGTATGTTGACTTTGAAAATGGCCAGTGGGCCACAGATGAC
ATCCCAGATGACTTGAATAGTATCCGCGCAGCACCAGGTGAGTTTCGAGC
CATCATGGAGATGCCCTCCTTCTACAGTCATGGCTTGCCACGGTGTTCAC
CCTACAAGAAACTGACAGAGTGCCAGCTGAAGAACCCCATCAGCGGGCTG
TTAGAATATGCCCAGTTCGCTAGTCAAACCTGTGAGTTCAACATGATAGA
GCAGAGTGGACCACCCCATGAACCTCGATTTAAATTCCAGGTTGTCATCA
ATGGCCGAGAGTTTCCCCCAGCTGAAGCTGGAAGCAAGAAAGTGGCCAAG
CAGGATGCAGCTATGAAAGCCATGACAATTCTGCTAGAGGAAGCCAAAGC
CAAGGACAGTGGAAAATCAGAAGAATCATCCCACTATTCCACAGAAAAAG
AATCAGAGAAGACCACAGAGTCCCAGACCCCCACCCCTTCAGCTACATCC
TTCTTTTCTGGGAAGAGCCCCGTCACCACACTGCTCGAGTGTATGCACAA
ATTGGGGAACTCCTGCGAATTCCGTCTCCTGTCCAAAGAAGGCCCTGCCC
ATGAGCCCAAGTTCCAGTACTGTGTTGCAGTGGGAGCCCAAACTTTCCCC
AGTGTGAGTGCCCCCAGCAAGAAAGTGGCAAAGCAGATGGCCGCAGAGGA
AGCCATGAAGGCCCTGCATGGGGAGGCAACCAACTCCATGACTTCTGATG
ACCAGCCCGAAGGTATGATCTCAGAGTCACTTGATAACTTGGAATCCGTG
ATGCCCAACAAGGTCAGGAAGATTGGCGAGCTCGTGAGATACTTGAACAC
CAATCCTGTGGGTGGCCTTTTGGAGTACGCCCGCTCCCATGGCTTTGCTG
CTGAATTCAAGTTGGTCGACCAGTCTGGACCTCCTCACGAGCCCAAGTTC
GTTTACCAAGCAAAAGTTGGGGGTCGCTGGTTCCCAGCCGTCTGCGCACA
CAGCAAGAAGCAAGGCAAGCAGGAAGCAGCAGATGCGGCTCTCCGTGTCT
TGATTGGGGAGAACGAGAAGGCAGAACGCATGGGTTTCACAGAGGTAACC
CCAGTGACAGGGGCCAGTCTCAGAAGAACTATGCTCCTCCTCTCAAGGTC
CCCAGAAGCACAGCCAAAGACACTCCCTCTCACTGGCAGCACCTTCCATG
ACCAGATAGCCATGCTGAGCCACCGGTGCTTCAACACTCTGACTAACAGC
TTCCAGCCTTCCTTGCTCGGCCGCAAGATTCTGGCCGCCATCGTTATGAA
AAAAGACTCTGAGGACATGGGTGTCGTCGTCAGCTTGGGAACAGGGAATC
GCTGTGTGAAAGGAGATTCTCTCAGCCTGAAGGGAGAAACTGTCAATGAC
TGCCATGCAGAAATCATCTCCCGGAGAGGCTTCATCAGGTTTCTCTACAG
TGAGTTAATGAAATACAACCCCCAGACTGCGAAGGATAGTATATTTGAAC
CTGCTAAGGGAGGAGAAAAGCTCCAAATAAAAAAGACTGTGTCATTCCAT
CTCTATATCAGCACCGCTCCATGTGGAGATGGTGCCCTCTTTGACAAGTC
CTGCAGCGACCGTGCTATGGAAAGCACAGACTCCCGCCACTACCCTGTCT
TTGAGAATCCCAAACAAGGCAAGCTCCGCACCAAGGTGGAAAACGGAGAA
GGCACAATCCCTGTGGAATCCAGTGACATTGTACCTACGTGGGATGGCAT
TCGGCTCGGGGAGAGACTCCGTACCATGTCCTGTAGTGACAAAATCCTAC
GCTGGAATGTGCTGGGCCTGCAAGGGGCACTGTTGACCCACTTCCTGCAG
CCCATTTATCTCAAATCTGTTACATTGGGTTACCTTTTCAGCCAAGGGCA
TCTGACCCGGGCTATTTGCTGTCGTGTGACAAGAGATGGGAGTGCATTTG
AGGATGGACTACGACATCCCTTTATTGTCAACCACCCCAAGGTTGGCAGA
GTCAGCGTATATGATTCCAAAAGGCAATCTGGGAAGACTAAGGAGACAAG
CGTCAACTGGTGTCTGGCTGATGGCTATGACCTGGAGATCCTGGATGGTA
CCAGAGGCACCGTGGATGGGCCACGGAATGAATTGTCCCGGGTCTCCAAA
AAGAACATTTTTCTTCTATTTAAGAAGCTCTGCTCCTTCCGTTACCGCAG
GGATCTACTGAGACTCTCCTATGGTGAGGCCAAGAAAGCTGCCCGTGACT
ACGAGACGGCCAAGAACTACTTCAAAAAAAGCTTGAAGGATATGGGCTAT
GGGAACTGGATTAGCAAACCCCAGGAGGAAAAGAACTTTTATCTCTGCCC
AGTATAG

SEQ ID NO: 43 - NHP ADAR1 - Transcript Variant X1 Amino Acid Sequence

MMSPICDHTIDSRLKVERATWWGRAGGGYRPHWQPPGVRPCPEEVQDPGY
SLNGYYPHPFQGYEHRKLRYQQPGPGSSPNSFLLKQIEFLKGQIPEAPVI
GKQTPSLPPSLPGLQPRFPVLPASSTRGRQVDIRGVPRCVHLGSQGLQRG
FQHPSPRGRIPPRRGVDCLSSQFQELSINQDQEQRILKFLEELGEGKATT
AHDLSWKLGAPKKEINRVLYSLAKKGKLQKEAGTPPLWKIAVSTQAWNQH
SRVVRPDGHSQGAPNSDPNLEPEDRNSTSVSEDLEPFISVSAQAWNQHSG
VVRPDGHSQGAPNSESSLEPEDRNSTSALEDPLEFFDMAEIKEKICDYLF
NVSDSSALNLAKNIGLTKARDINAVLIDMERQGDVYRQGTTPPIWHLTDK
KRERMQIKRNTNSVPETAPAAIPETKRNTEFLTCNIPTSNASNNIVTTEK
VENGQEPVIKLENSQEARPEPVRLKPPVHNNGPSKAGYVDFENGQWATDD
IPDDLNSIRAAPGEFRAIMEMPSFYSHGLPRCSPYKKLTECQLKNPISGL
LEYAQFASQTCEFNMIEQSGPPHEPRFKFQVVINGREFPPAEAGSKKVAK
QDAAMKAMTILLEEAKAKDSGKSEESSHYSTEKESEKTTESQTPTPSATS
FFSGKSPVTTLLECMHKLGNSCEFRLLSKEGPAHEPKFQYCVAVGAQTFP
SVSAPSKKVAKQMAAEEAMKALHGEATNSMTSDDQPEGMISESLDNLESV
MPNKVRKIGELVRYLNTNPVGGLLEYARSHGFAAEFKLVDQSGPPHEPKF
VYQAKVGGRWFPAVCAHSKKQGKQEAADAALRVLIGENEKAERMGFTEVT
PVTGASLRRTMLLLSRSPEAQPKTLPLTGSTFHDQIAMLSHRCFNTLTNS
FQPSLLGRKILAAIVMKKDSEDMGVVVSLGTGNRCVKGDSLSLKGETVND
CHAEIISRRGFIRFLYSELMKYNPQTAKDSIFEPAKGGEKLQIKKTVSFH
LYISTAPCGDGALFDKSCSDRAMESTDSRHYPVFENPKQGKLRTKVENGE
GTIPVESSDIVPTWDGIRLGERLRTMSCSDKILRWNVLGLQGALLTHFLQ
PIYLKSVTLGYLFSQGHLTRAICCRVTRDGSAFEDGLRHPFIVNHPKVGR
VSVYDSKRQSGKTKETSVNWCLADGYDLEILDGTRGTVDGPRNELSRVSK
KNIFLLFKKLCSFRYRRDLLRLSYGEAKKAARDYETAKNYFKKSLKDMGY
GNWISKPQEEKNFYLCPV

In certain embodiments, an ADAR1 polynucleotide or an ADAR1 gene incorporated into a non-human animal (e.g., a rodent, e.g., a rat or mouse) is represented by or comprises a sequence encoding a non-human primate (NHP) ADAR1 transcript or a characteristic portion thereof. In some embodiments, a NHP ADAR1 transcript variant is transcript variant X2. In certain embodiments, an ADAR1 polynucleotide or a NHP ADAR1 gene comprises, or consists of, a nucleotide sequence that is the same as, differs by no more than 1, 2, 3, 4, 5, 6, 7, 8, 9, or 10 nucleotides from, or has at least has at least 90%, 92%, 94%, 96%, 98%, or 99% homology with, the nucleotide sequence of SEQ ID NO: 44 or SEQ ID NO: 45 or a characteristic portion thereof. In some embodiments, an ADAR1 polynucleotide or a NHP ADAR1 gene comprises, or consists of, a nucleotide sequence that is the same as the nucleotide sequence of SEQ ID NO: 44 or SEQ ID NO: 45 or a characteristic portion thereof. In certain embodiments, an encoded ADAR1 Amino Acid sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the Amino Acid sequence of SEQ ID NO: 46 or a characteristic portion thereof. In certain embodiments, the amino acid sequence of an encoded and/or expressed ADAR1 polypeptide or a characteristic portion thereof, is or comprises a sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the amino acid sequence of SEQ ID NO: 46 or a characteristic portion thereof.

SEQ ID NO: 44 - NHP ADAR1 - Transcript Variant X2 cDNA Sequence

ACGCCCGGCGCGCCAATGATGTCACCAATCTGCGACCACACCATTGATTC
CCGACTGAAGGTAGAGAGGGCTACGTGGTGGGGGAGGGCGGGGGGAGGGT
ATCGGCCGCACTGGCAGCCTCCGGGTGTTCGGCCGTGTCCGGAGGAAGTA
CAAGACCCGGGGTATTCCCTCAACGGATACTACCCCCATCCATTTCAAGG
CTATGAGCACAGAAAGCTCAGATACCAGCAGCCTGGGCCAGGATCTTCCC
CCAATAGTTTCCTGCTTAAGCAAATAGAGTTTCTCAAGGGGCAGATCCCA
GAAGCGCCGGTGATTGGAAAGCAGACACCATCACTGCCACCTTCCCTCCC
AGGACTCCAGCCAAGGTTTCCAGTACTACCTGCCTCTAGTACCAGAGGCA
GGCAAGTGGATATCAGGGGTGTCCCCAGGTGCGTGCATCTCGGAAGTCAG
GGGCTCCAGAGAGGGTTCCAGCATCCTTCACCACGTGGCAGGATTCCGCC
ACGGAGAGGTGTTGATTGCCTTTCCTCACAATTCCAGGAACTGAGTATCA
ACCAAGATCAGGAACAAAGGATCTTAAAGTTCTTGGAAGAGCTTGGGGAA
GGGAAGGCCACTACAGCACATGATCTGTCTTGGAAACTGGGGGCTCCAAA
GAAAGAAATCAATCGAGTTTTATACTCCCTGGCAAAGAAGGGCAAGCTAC
AGAAAGAGGCAGGAACACCCCCTTTGTGGAAAATCGCGGTCTCCACTCAG
GCTTGGAACCAGCACAGCAGAGTGGTAAGACCAGACGGTCATAGCCAAGG
AGCCCCAAACTCAGACCCAAATTTGGAACCTGAAGACAGAAACTCCACAT
CTGTCTCAGAAGATCTTGAGCCTTTTATCTCAGTCTCAGCTCAGGCTTGG
AACCAGCACAGCGGAGTGGTAAGACCAGACGGTCATAGCCAAGGAGCCCC
AAACTCAGAGTCGAGTTTGGAACCTGAAGACAGAAACTCCACATCTGCCT
TGGAAGATCCTCTTGAGTTTTTTGACATGGCTGAGATCAAGGAGAAAATC
TGCGACTATCTCTTCAATGTGTCTGACTCCTCTGCCCTGAATTTGGCTAA
AAATATTGGCCTCACCAAGGCCCGAGATATAAATGCCGTGCTAATTGACA
TGGAAAGGCAGGGGGATGTCTATAGACAAGGGACAACCCCTCCCATATGG
CATTTGACAGACAAGAAGCGAGAGAGGATGCAAATCAAGAGAAATACGAA
CAGTGTTCCTGAAACCGCTCCAGCTGCAATCCCTGAGACCAAAAGAAACA
CAGAGTTCCTCACCTGTAATATACCCACATCAAATGCCTCAAATAACATA
GTAACCACCGAAAAAGTGGAGAATGGGCAGGAACCTGTCATAAAGTTAGA
AAACAGTCAGGAGGCAAGACCAGAACCAGTAAGACTGAAACCACCTGTTC
ATAACAATGGCCCCTCAAAAGCAGGGTATGTTGACTTTGAAAATGGCCAG
TGGGCCACAGATGACATCCCAGATGACTTGAATAGTATCCGCGCAGCACC
AGGTGAGTTTCGAGCCATCATGGAGATGCCCTCCTTCTACAGTCATGGCT
TGCCACGGTGTTCACCCTACAAGAAACTGACAGAGTGCCAGCTGAAGAAC
CCCATCAGCGGGCTGTTAGAATATGCCCAGTTCGCTAGTCAAACCTGTGA
GTTCAACATGATAGAGCAGAGTGGACCACCCCATGAACCTCGATTTAAAT
TCCAGGTTGTCATCAATGGCCGAGAGTTTCCCCCAGCTGAAGCTGGAAGC
AAGAAAGTGGCCAAGCAGGATGCAGCTATGAAAGCCATGACAATTCTGCT
AGAGGAAGCCAAAGCCAAGGACAGTGGAAAATCAGAAGAATCATCCCACT
ATTCCACAGAAAAAGAATCAGAGAAGACCACAGAGTCCCAGACCCCCACC
CCTTCAGCTACATCCTTCTTTTCTGGGAAGAGCCCCGTCACCACACTGCT
CGAGTGTATGCACAAATTGGGGAACTCCTGCGAATTCCGTCTCCTGTCCA
AAGAAGGCCCTGCCCATGAGCCCAAGTTCCAGTACTGTGTTGCAGTGGGA
GCCCAAACTTTCCCCAGTGTGAGTGCCCCCAGCAAGAAAGTGGCAAAGCA
GATGGCCGCAGAGGAAGCCATGAAGGCCCTGCATGGGGAGGCAACCAACT
CCATGACTTCTGATGACCAGCCCGAAGGTATGATCTCAGAGTCACTTGAT
AACTTGGAATCCGTGATGCCCAACAAGGTCAGGAAGATTGGCGAGCTCGT
GAGATACTTGAACACCAATCCTGTGGGTGGCCTTTTGGAGTACGCCCGCT
CCCATGGCTTTGCTGCTGAATTCAAGTTGGTCGACCAGTCTGGACCTCCT
CACGAGCCCAAGTTCGTTTACCAAGCAAAAGTTGGGGGTCGCTGGTTCCC
AGCCGTCTGCGCACACAGCAAGAAGCAAGGCAAGCAGGAAGCAGCAGATG
CGGCTCTCCGTGTCTTGATTGGGGAGAACGAGAAGGCAGAACGCATGGGT
TTCACAGAGCTCCCTCTCACTGGCAGCACCTTCCATGACCAGATAGCCAT
GCTGAGCCACCGGTGCTTCAACACTCTGACTAACAGCTTCCAGCCTTCCT
TGCTCGGCCGCAAGATTCTGGCCGCCATCGTTATGAAAAAAGACTCTGAG
GACATGGGTGTCGTCGTCAGCTTGGGAACAGGGAATCGCTGTGTGAAAGG
AGATTCTCTCAGCCTGAAGGGAGAAACTGTCAATGACTGCCATGCAGAAA
TCATCTCCCGGAGAGGCTTCATCAGGTTTCTCTACAGTGAGTTAATGAAA
TACAACCCCCAGACTGCGAAGGATAGTATATTTGAACCTGCTAAGGGAGG
AGAAAAGCTCCAAATAAAAAAGACTGTGTCATTCCATCTCTATATCAGCA
CCGCTCCATGTGGAGATGGTGCCCTCTTTGACAAGTCCTGCAGCGACCGT
GCTATGGAAAGCACAGACTCCCGCCACTACCCTGTCTTTGAGAATCCCAA
ACAAGGCAAGCTCCGCACCAAGGTGGAAAACGGAGAAGGCACAATCCCTG
TGGAATCCAGTGACATTGTACCTACGTGGGATGGCATTCGGCTCGGGGAG
AGACTCCGTACCATGTCCTGTAGTGACAAAATCCTACGCTGGAATGTGCT
GGGCCTGCAAGGGGCACTGTTGACCCACTTCCTGCAGCCCATTTATCTCA
AATCTGTTACATTGGGTTACCTTTTCAGCCAAGGGCATCTGACCCGGGCT
ATTTGCTGTCGTGTGACAAGAGATGGGAGTGCATTTGAGGATGGACTACG
ACATCCCTTTATTGTCAACCACCCCAAGGTTGGCAGAGTCAGCGTATATG
ATTCCAAAAGGCAATCTGGGAAGACTAAGGAGACAAGCGTCAACTGGTGT
CTGGCTGATGGCTATGACCTGGAGATCCTGGATGGTACCAGAGGCACCGT
GGATGGGCCACGGAATGAATTGTCCCGGGTCTCCAAAAAGAACATTTTTC
TTCTATTTAAGAAGCTCTGCTCCTTCCGTTACCGCAGGGATCTACTGAGA
CTCTCCTATGGTGAGGCCAAGAAAGCTGCCCGTGACTACGAGACGGCCAA
GAACTACTTCAAAAAAAGCTTGAAGGATATGGGCTATGGGAACTGGATTA
GCAAACCCCAGGAGGAAAAGAACTTTTATCTCTGCCCAGTATAGTATGCT
CCGGTGACAGATGGATTAGGGTGTGTCATACTAGGGTGAGAGAGAGGTAG
GTCGTAGCGTTCCTCATCACATGGTCAGGGGATTTTTTTTTCCTCCTTTT
TTTTCTTTTTAAGCCATAATTGGTGATACTGAAAACTTTGGGTTCCCATT
TATCCTGCTTTCTTTGGGATTGCTAGGCAAGGTCTGGCCAGGCCCCCCTT
TTTTTCCCCCAAGTGAAGAGGTAGAAACCTAAGAAGTTATCTTTTCTTTC
TATAACTCAAAGCATACATAGTCACTGAGCACCTGCAGTCCATTTCCTCT
TAAAAGTTTTTTTTTTTTTCCATTTCCTTTCCCTTTGTGTTTGCTACACT
GACCTCTTGTGGTCTTGATTAGGTTTCAGTCAATTCTGGATCATGTCCGA
GACTGATGATTTCTTTTGTGGATTACGCAGACCCCTCTACTTCCCCCTTC
CCCTTCTGAGGTTCTTTCCTTGTGATCAGAATTTCTCCTTTTCTCCCTCA
GAGGGTAAAAAGGTGAACTTAAAGGATCTGGTGAAACATTTGTAAGGGTA
GGAGTTGAAAACTGCAGTTCCCAGTGTCACGGAAGTGTGATTGGAGCCTA
CAGATAATGCCCTGCCATCCTCCCATCCTGCACTTTAGCCAGCTGCAGGG
CGGGCAAGGCAAGGAAAGCTGCTTCCCTGGAAGTGTGCCCCTTTCCCCGG
CAGCAGGGAAGTCTAGAACCAGCCAGACTGGGTTAAGGGAGTTGCCCAAG
CCATAGCGGAGGTTTCACCCAGCAAGGTGACACAGACCACTTCCCAGGAG
CACGGGCATGCCTTGGGATATTGCCAAGCTTCCAGCTGCCTCTTCTCCTA
AAGCACTCCTAGGAATTTGCCCTGCAAATGCTGGGCGTGCACCCTAGCCA
ACGTAGGAAAAAATCCAGGACAAATCCTAGAGGGTAGAAAATCATCTCTG
CTCAGATAATCATGACTTAGCAAGAATAAGGGCAAAAAATCCTGTTGGCT
CACGTCACTGTTCTACCCTGCATAATATCTCTCATGACAGTGACACCAAG
AGAAGTTGACTAAGTCATATGTAAATTAGGAGTGTTTAAAAGAATGCCAT
AGATGTTGATTCTTAACTGCTACAGACAACCTATAATTGAGCAGATTTAA
AATTCAGGCATACTTTTCCATTTATCCAAGTGCTTTCATTTTTCCAGATG
GCTTCAGAAGTAGGCTCGTGGGCAGAGCGCAGACCTGATCTTTATAGTGT
TGACATAGAAAGCAGTAGTAGTGGGTGAAAGGACAGGTTGTCTTCAAACT
CTGTGAGGTGGAATCTTTTGTCTACACCTCCATGAACATTGACTCGTGTG
TTCAGAGCCTTTGGCCTCCCTGTGGAGTCTGGCTCTCTGGCTCCTGTGCA
TTCTTTGAATAGTCACTCGTAAAAATTGTCAATGCTTGAAACTATTTCTT
TTACTCATGTTGAAGGGACTTTGTTGACTTTTAGAGTGTTGGTCATGACT
CCAAGAGCAGAGCAGGGAAGAGCCCAAGCATAGACTTGAGTGCCGTGATG
GCTGCGGTCCAGTTTTGTGATTCTGCTTTTACGTGTTACTTGGTAACAGT
CAGCTAGACACACTCAGGAGGACTACTGAGGCTCTGCGACCTTCAGGACC
TGAGCCTGCCTCTCTCCTTTAGATGACAGACCTTCATCTGAGAATGTGCT
GAGCCAGCACCCTCAGATGATTTCCCTCCAAACTGCTGACTAGGTCACCC
TCTGTCTGGTAGAGACATTGACATCTTTGCTTTTATTCTATACCCTCGGT
ACTTTTGACCAAAAAATGACCAAAATAAGAAAATGCAACTTTAAAAAATA
GACTAAGGATGCCTTTGCAGAACACCAAAGCATCCCAAGGAACCGGTAGG
GAAATGATGCCTGTCTCCTGGGGTAGAAGAGGCCTGCTCCCTGGCTCTGG
GTCTGCTGGGGGCACAGTAAATCAGTCTTGGCACCCACATCCAGGTCAGA
GAGGTCTGTGGTTCTCAGCATCAGAAGGCAGCGCAGCCCCTCTCCAGGCT
ACAGGGTTGTCACCTGCTGAGTCCTCAAGCTGTTTGGCCTCTCTGGTCCG
TCTTGGGCATTAGATTCTCCAGCAGAGCTCTGGCCAGCTGCCTCCTCTTT
AACTGGGAACACGGGCTCTCACAAGATCAGAACCCCCACTCACCCCTGAG
ATCTTATCTAGCAAGCCTGTAGTATTCCGTTTCTGTTGTAGGAAGAGCGC
GAGGCATCCCTGAATTCCATGCATCTGCTGGAAATGAGCCGTGTCAGATC
GCACACCCCCGCGCCCCCATGCCCCTCTGAGTCACACGGGACAGAGGAGG
CAGAGCTTCCGCCCACTGTTATCTTCAATTTCTTTGTCCAGTCTTTTGTT
TTTAATAAGCAGTGCCCCTCCCCACTCTTCTTTTTAATGATTTTTGTAGT
TGATTTGTCTGAACTGTGGCTACTGTGCATTCCTTGAATAATCACTTGTA
AAAATTGTCAGTGCTTGAAGCTGTTTCCTTTACTCACATTGAAGGGACTT
TGTTGGTTTTTTGGAGTCTTGGTTTTGACTCCAAGAACAGAGTCAGGAAG
ACCCCAAGCATAGACTTGGGTACCGTGAAAATGGCTGCAGTCCAGTTTTA
TGATTCCGCTTTTATGTGTCCCTTGATAACAGTGACTTAACAATATACAT
TCCTCATAAATAAAAAAAAAACCAAGAATCTGAATTCTTA

SEQ ID NO: 45 - NHP ADAR1 - Transcript Variant X2 Coding Sequence

ATGATGTCACCAATCTGCGACCACACCATTGATTCCCGACTGAAGGTAGA
GAGGGCTACGTGGTGGGGGAGGGCGGGGGGAGGGTATCGGCCGCACTGGC
AGCCTCCGGGTGTTCGGCCGTGTCCGGAGGAAGTACAAGACCCGGGGTAT
TCCCTCAACGGATACTACCCCCATCCATTTCAAGGCTATGAGCACAGAAA
GCTCAGATACCAGCAGCCTGGGCCAGGATCTTCCCCCAATAGTTTCCTGC
TTAAGCAAATAGAGTTTCTCAAGGGGCAGATCCCAGAAGCGCCGGTGATT
GGAAAGCAGACACCATCACTGCCACCTTCCCTCCCAGGACTCCAGCCAAG
GTTTCCAGTACTACCTGCCTCTAGTACCAGAGGCAGGCAAGTGGATATCA
GGGGTGTCCCCAGGTGCGTGCATCTCGGAAGTCAGGGGCTCCAGAGAGGG
TTCCAGCATCCTTCACCACGTGGCAGGATTCCGCCACGGAGAGGTGTTGA
TTGCCTTTCCTCACAATTCCAGGAACTGAGTATCAACCAAGATCAGGAAC
AAAGGATCTTAAAGTTCTTGGAAGAGCTTGGGGAAGGGAAGGCCACTACA
GCACATGATCTGTCTTGGAAACTGGGGGCTCCAAAGAAAGAAATCAATCG
AGTTTTATACTCCCTGGCAAAGAAGGGCAAGCTACAGAAAGAGGCAGGAA
CACCCCCTTTGTGGAAAATCGCGGTCTCCACTCAGGCTTGGAACCAGCAC
AGCAGAGTGGTAAGACCAGACGGTCATAGCCAAGGAGCCCCAAACTCAGA
CCCAAATTTGGAACCTGAAGACAGAAACTCCACATCTGTCTCAGAAGATC
TTGAGCCTTTTATCTCAGTCTCAGCTCAGGCTTGGAACCAGCACAGCGGA
GTGGTAAGACCAGACGGTCATAGCCAAGGAGCCCCAAACTCAGAGTCGAG
TTTGGAACCTGAAGACAGAAACTCCACATCTGCCTTGGAAGATCCTCTTG
AGTTTTTTGACATGGCTGAGATCAAGGAGAAAATCTGCGACTATCTCTTC
AATGTGTCTGACTCCTCTGCCCTGAATTTGGCTAAAAATATTGGCCTCAC
CAAGGCCCGAGATATAAATGCCGTGCTAATTGACATGGAAAGGCAGGGGG
ATGTCTATAGACAAGGGACAACCCCTCCCATATGGCATTTGACAGACAAG
AAGCGAGAGAGGATGCAAATCAAGAGAAATACGAACAGTGTTCCTGAAAC
CGCTCCAGCTGCAATCCCTGAGACCAAAAGAAACACAGAGTTCCTCACCT
GTAATATACCCACATCAAATGCCTCAAATAACATAGTAACCACCGAAAAA
GTGGAGAATGGGCAGGAACCTGTCATAAAGTTAGAAAACAGTCAGGAGGC
AAGACCAGAACCAGTAAGACTGAAACCACCTGTTCATAACAATGGCCCCT
CAAAAGCAGGGTATGTTGACTTTGAAAATGGCCAGTGGGCCACAGATGAC
ATCCCAGATGACTTGAATAGTATCCGCGCAGCACCAGGTGAGTTTCGAGC
CATCATGGAGATGCCCTCCTTCTACAGTCATGGCTTGCCACGGTGTTCAC
CCTACAAGAAACTGACAGAGTGCCAGCTGAAGAACCCCATCAGCGGGCTG
TTAGAATATGCCCAGTTCGCTAGTCAAACCTGTGAGTTCAACATGATAGA
GCAGAGTGGACCACCCCATGAACCTCGATTTAAATTCCAGGTTGTCATCA
ATGGCCGAGAGTTTCCCCCAGCTGAAGCTGGAAGCAAGAAAGTGGCCAAG
CAGGATGCAGCTATGAAAGCCATGACAATTCTGCTAGAGGAAGCCAAAGC
CAAGGACAGTGGAAAATCAGAAGAATCATCCCACTATTCCACAGAAAAAG
AATCAGAGAAGACCACAGAGTCCCAGACCCCCACCCCTTCAGCTACATCC
TTCTTTTCTGGGAAGAGCCCCGTCACCACACTGCTCGAGTGTATGCACAA
ATTGGGGAACTCCTGCGAATTCCGTCTCCTGTCCAAAGAAGGCCCTGCCC
ATGAGCCCAAGTTCCAGTACTGTGTTGCAGTGGGAGCCCAAACTTTCCCC
AGTGTGAGTGCCCCCAGCAAGAAAGTGGCAAAGCAGATGGCCGCAGAGGA
AGCCATGAAGGCCCTGCATGGGGAGGCAACCAACTCCATGACTTCTGATG
ACCAGCCCGAAGGTATGATCTCAGAGTCACTTGATAACTTGGAATCCGTG
ATGCCCAACAAGGTCAGGAAGATTGGCGAGCTCGTGAGATACTTGAACAC
CAATCCTGTGGGTGGCCTTTTGGAGTACGCCCGCTCCCATGGCTTTGCTG
CTGAATTCAAGTTGGTCGACCAGTCTGGACCTCCTCACGAGCCCAAGTTC
GTTTACCAAGCAAAAGTTGGGGGTCGCTGGTTCCCAGCCGTCTGCGCACA
CAGCAAGAAGCAAGGCAAGCAGGAAGCAGCAGATGCGGCTCTCCGTGTCT
TGATTGGGGAGAACGAGAAGGCAGAACGCATGGGTTTCACAGAGCTCCCT
CTCACTGGCAGCACCTTCCATGACCAGATAGCCATGCTGAGCCACCGGTG
CTTCAACACTCTGACTAACAGCTTCCAGCCTTCCTTGCTCGGCCGCAAGA
TTCTGGCCGCCATCGTTATGAAAAAAGACTCTGAGGACATGGGTGTCGTC
GTCAGCTTGGGAACAGGGAATCGCTGTGTGAAAGGAGATTCTCTCAGCCT
GAAGGGAGAAACTGTCAATGACTGCCATGCAGAAATCATCTCCCGGAGAG
GCTTCATCAGGTTTCTCTACAGTGAGTTAATGAAATACAACCCCCAGACT
GCGAAGGATAGTATATTTGAACCTGCTAAGGGAGGAGAAAAGCTCCAAAT
AAAAAAGACTGTGTCATTCCATCTCTATATCAGCACCGCTCCATGTGGAG
ATGGTGCCCTCTTTGACAAGTCCTGCAGCGACCGTGCTATGGAAAGCACA
GACTCCCGCCACTACCCTGTCTTTGAGAATCCCAAACAAGGCAAGCTCCG
CACCAAGGTGGAAAACGGAGAAGGCACAATCCCTGTGGAATCCAGTGACA
TTGTACCTACGTGGGATGGCATTCGGCTCGGGGAGAGACTCCGTACCATG
TCCTGTAGTGACAAAATCCTACGCTGGAATGTGCTGGGCCTGCAAGGGGC
ACTGTTGACCCACTTCCTGCAGCCCATTTATCTCAAATCTGTTACATTGG
GTTACCTTTTCAGCCAAGGGCATCTGACCCGGGCTATTTGCTGTCGTGTG
ACAAGAGATGGGAGTGCATTTGAGGATGGACTACGACATCCCTTTATTGT
CAACCACCCCAAGGTTGGCAGAGTCAGCGTATATGATTCCAAAAGGCAAT
CTGGGAAGACTAAGGAGACAAGCGTCAACTGGTGTCTGGCTGATGGCTAT
GACCTGGAGATCCTGGATGGTACCAGAGGCACCGTGGATGGGCCACGGAA
TGAATTGTCCCGGGTCTCCAAAAAGAACATTTTTCTTCTATTTAAGAAGC
TCTGCTCCTTCCGTTACCGCAGGGATCTACTGAGACTCTCCTATGGTGAG
GCCAAGAAAGCTGCCCGTGACTACGAGACGGCCAAGAACTACTTCAAAAA
AAGCTTGAAGGATATGGGCTATGGGAACTGGATTAGCAAACCCCAGGAGG
AAAAGAACTTTTATCTCTGCCCAGTATAG

SEQ ID NO: 46 - NHP ADAR1 - Transcript Variant X2 Amino Acid Sequence

MMSPICDHTIDSRLKVERATWWGRAGGGYRPHWQPPGVRPCPEEVQDPGY
SLNGYYPHPFQGYEHRKLRYQQPGPGSSPNSFLLKQIEFLKGQIPEAPVI
GKQTPSLPPSLPGLQPRFPVLPASSTRGRQVDIRGVPRCVHLGSQGLQRG
FQHPSPRGRIPPRRGVDCLSSQFQELSINQDQEQRILKFLEELGEGKATT
AHDLSWKLGAPKKEINRVLYSLAKKGKLQKEAGTPPLWKIAVSTQAWNQH
SRVVRPDGHSQGAPNSDPNLEPEDRNSTSVSEDLEPFISVSAQAWNQHSG
VVRPDGHSQGAPNSESSLEPEDRNSTSALEDPLEFFDMAEIKEKICDYLF
NVSDSSALNLAKNIGLTKARDINAVLIDMERQGDVYRQGTTPPIWHLTDK
KRERMQIKRNTNSVPETAPAAIPETKRNTEFLTCNIPTSNASNNIVTTEK
VENGQEPVIKLENSQEARPEPVRLKPPVHNNGPSKAGYVDFENGQWATDD
IPDDLNSIRAAPGEFRAIMEMPSFYSHGLPRCSPYKKLTECQLKNPISGL
LEYAQFASQTCEFNMIEQSGPPHEPRFKFQVVINGREFPPAEAGSKKVAK
QDAAMKAMTILLEEAKAKDSGKSEESSHYSTEKESEKTTESQTPTPSATS
FFSGKSPVTTLLECMHKLGNSCEFRLLSKEGPAHEPKFQYCVAVGAQTFP
SVSAPSKKVAKQMAAEEAMKALHGEATNSMTSDDQPEGMISESLDNLESV
MPNKVRKIGELVRYLNTNPVGGLLEYARSHGFAAEFKLVDQSGPPHEPKF
VYQAKVGGRWFPAVCAHSKKQGKQEAADAALRVLIGENEKAERMGFTELP
LTGSTFHDQIAMLSHRCFNTLTNSFQPSLLGRKILAAIVMKKDSEDMGVV
VSLGTGNRCVKGDSLSLKGETVNDCHAEIISRRGFIRFLYSELMKYNPQT
AKDSIFEPAKGGEKLQIKKTVSFHLYISTAPCGDGALFDKSCSDRAMEST
DSRHYPVFENPKQGKLRTKVENGEGTIPVESSDIVPTWDGIRLGERLRTM
SCSDKILRWNVLGLQGALLTHFLQPIYLKSVTLGYLFSQGHLTRAICCRV
TRDGSAFEDGLRHPFIVNHPKVGRVSVYDSKRQSGKTKETSVNWCLADGY
DLEILDGTRGTVDGPRNELSRVSKKNIFLLFKKLCSFRYRRDLLRLSYGE
AKKAARDYETAKNYFKKSLKDMGYGNWISKPQEEKNFYLCPV

In certain embodiments, an ADAR1 polynucleotide or an ADAR1 gene incorporated into a non-human animal (e.g., a rodent, e.g., a rat or mouse) is represented by or comprises a sequence encoding a non-human primate (NHP) ADAR1 transcript variant or a characteristic portion thereof. In some embodiments, a NHP ADAR1 transcript variant is transcript variant X3. In certain embodiments, ADAR1 polynucleotide or a NHP ADAR1 gene comprises, or consists of, a nucleotide sequence that is the same as, differs by no more than 1, 2, 3, 4, 5, 6, 7, 8, 9, or 10 nucleotides from, or has at least has at least 90%, 92%, 94%, 96%, 98%, or 99% homology with, the nucleotide sequence of SEQ ID NO: 47 or SEQ ID NO: 48 or a characteristic portion thereof. In some embodiments, an ADAR1 polynucleotide or a NHP ADAR1 gene comprises, or consists of, a nucleotide sequence that is the same as the nucleotide sequence of SEQ ID NO: 47 or SEQ ID NO: 48 or a characteristic portion thereof. In certain embodiments, an encoded ADAR1 Amino Acid sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the Amino Acid sequence of SEQ ID NO: 49 or a characteristic portion thereof. In certain embodiments, the amino acid sequence of an encoded and/or expressed ADAR1 polypeptide or a characteristic portion thereof, is or comprises a sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the amino acid sequence of SEQ ID NO: 49 or a characteristic portion thereof.

SEQ ID NO: 47 - NHP ADAR1 - Transcript Variant X3 cDNA Sequence

ACGCCCGGCGCGCCAATGATGTCACCAATCTGCGACCACACCATTGATTC
CCGACTGAAGGGGTATTCCCTCAACGGATACTACCCCCATCCATTTCAAG
GCTATGAGCACAGAAAGCTCAGATACCAGCAGCCTGGGCCAGGATCTTCC
CCCAATAGTTTCCTGCTTAAGCAAATAGAGTTTCTCAAGGGGCAGATCCC
AGAAGCGCCGGTGATTGGAAAGCAGACACCATCACTGCCACCTTCCCTCC
CAGGACTCCAGCCAAGGTTTCCAGTACTACCTGCCTCTAGTACCAGAGGC
AGGCAAGTGGATATCAGGGGTGTCCCCAGGTGCGTGCATCTCGGAAGTCA
GGGGCTCCAGAGAGGGTTCCAGCATCCTTCACCACGTGGCAGGATTCCGC
CACGGAGAGGTGTTGATTGCCTTTCCTCACAATTCCAGGAACTGAGTATC
AACCAAGATCAGGAACAAAGGATCTTAAAGTTCTTGGAAGAGCTTGGGGA
AGGGAAGGCCACTACAGCACATGATCTGTCTTGGAAACTGGGGGCTCCAA
AGAAAGAAATCAATCGAGTTTTATACTCCCTGGCAAAGAAGGGCAAGCTA
CAGAAAGAGGCAGGAACACCCCCTTTGTGGAAAATCGCGGTCTCCACTCA
GGCTTGGAACCAGCACAGCAGAGTGGTAAGACCAGACGGTCATAGCCAAG
GAGCCCCAAACTCAGACCCAAATTTGGAACCTGAAGACAGAAACTCCACA
TCTGTCTCAGAAGATCTTGAGCCTTTTATCTCAGTCTCAGCTCAGGCTTG
GAACCAGCACAGCGGAGTGGTAAGACCAGACGGTCATAGCCAAGGAGCCC
CAAACTCAGAGTCGAGTTTGGAACCTGAAGACAGAAACTCCACATCTGCC
TTGGAAGATCCTCTTGAGTTTTTTGACATGGCTGAGATCAAGGAGAAAAT
CTGCGACTATCTCTTCAATGTGTCTGACTCCTCTGCCCTGAATTTGGCTA
AAAATATTGGCCTCACCAAGGCCCGAGATATAAATGCCGTGCTAATTGAC
ATGGAAAGGCAGGGGGATGTCTATAGACAAGGGACAACCCCTCCCATATG
GCATTTGACAGACAAGAAGCGAGAGAGGATGCAAATCAAGAGAAATACGA
ACAGTGTTCCTGAAACCGCTCCAGCTGCAATCCCTGAGACCAAAAGAAAC
ACAGAGTTCCTCACCTGTAATATACCCACATCAAATGCCTCAAATAACAT
AGTAACCACCGAAAAAGTGGAGAATGGGCAGGAACCTGTCATAAAGTTAG
AAAACAGTCAGGAGGCAAGACCAGAACCAGTAAGACTGAAACCACCTGTT
CATAACAATGGCCCCTCAAAAGCAGGGTATGTTGACTTTGAAAATGGCCA
GTGGGCCACAGATGACATCCCAGATGACTTGAATAGTATCCGCGCAGCAC
CAGGTGAGTTTCGAGCCATCATGGAGATGCCCTCCTTCTACAGTCATGGC
TTGCCACGGTGTTCACCCTACAAGAAACTGACAGAGTGCCAGCTGAAGAA
CCCCATCAGCGGGCTGTTAGAATATGCCCAGTTCGCTAGTCAAACCTGTG
AGTTCAACATGATAGAGCAGAGTGGACCACCCCATGAACCTCGATTTAAA
TTCCAGGTTGTCATCAATGGCCGAGAGTTTCCCCCAGCTGAAGCTGGAAG
CAAGAAAGTGGCCAAGCAGGATGCAGCTATGAAAGCCATGACAATTCTGC
TAGAGGAAGCCAAAGCCAAGGACAGTGGAAAATCAGAAGAATCATCCCAC
TATTCCACAGAAAAAGAATCAGAGAAGACCACAGAGTCCCAGACCCCCAC
CCCTTCAGCTACATCCTTCTTTTCTGGGAAGAGCCCCGTCACCACACTGC
TCGAGTGTATGCACAAATTGGGGAACTCCTGCGAATTCCGTCTCCTGTCC
AAAGAAGGCCCTGCCCATGAGCCCAAGTTCCAGTACTGTGTTGCAGTGGG
AGCCCAAACTTTCCCCAGTGTGAGTGCCCCCAGCAAGAAAGTGGCAAAGC
AGATGGCCGCAGAGGAAGCCATGAAGGCCCTGCATGGGGAGGCAACCAAC
TCCATGACTTCTGATGACCAGCCCGAAGGTATGATCTCAGAGTCACTTGA
TAACTTGGAATCCGTGATGCCCAACAAGGTCAGGAAGATTGGCGAGCTCG
TGAGATACTTGAACACCAATCCTGTGGGTGGCCTTTTGGAGTACGCCCGC
TCCCATGGCTTTGCTGCTGAATTCAAGTTGGTCGACCAGTCTGGACCTCC
TCACGAGCCCAAGTTCGTTTACCAAGCAAAAGTTGGGGGTCGCTGGTTCC
CAGCCGTCTGCGCACACAGCAAGAAGCAAGGCAAGCAGGAAGCAGCAGAT
GCGGCTCTCCGTGTCTTGATTGGGGAGAACGAGAAGGCAGAACGCATGGG
TTTCACAGAGGTAACCCCAGTGACAGGGGCCAGTCTCAGAAGAACTATGC
TCCTCCTCTCAAGGTCCCCAGAAGCACAGCCAAAGACACTCCCTCTCACT
GGCAGCACCTTCCATGACCAGATAGCCATGCTGAGCCACCGGTGCTTCAA
CACTCTGACTAACAGCTTCCAGCCTTCCTTGCTCGGCCGCAAGATTCTGG
CCGCCATCGTTATGAAAAAAGACTCTGAGGACATGGGTGTCGTCGTCAGC
TTGGGAACAGGGAATCGCTGTGTGAAAGGAGATTCTCTCAGCCTGAAGGG
AGAAACTGTCAATGACTGCCATGCAGAAATCATCTCCCGGAGAGGCTTCA
TCAGGTTTCTCTACAGTGAGTTAATGAAATACAACCCCCAGACTGCGAAG
GATAGTATATTTGAACCTGCTAAGGGAGGAGAAAAGCTCCAAATAAAAAA
GACTGTGTCATTCCATCTCTATATCAGCACCGCTCCATGTGGAGATGGTG
CCCTCTTTGACAAGTCCTGCAGCGACCGTGCTATGGAAAGCACAGACTCC
CGCCACTACCCTGTCTTTGAGAATCCCAAACAAGGCAAGCTCCGCACCAA
GGTGGAAAACGGAGAAGGCACAATCCCTGTGGAATCCAGTGACATTGTAC
CTACGTGGGATGGCATTCGGCTCGGGGAGAGACTCCGTACCATGTCCTGT
AGTGACAAAATCCTACGCTGGAATGTGCTGGGCCTGCAAGGGGCACTGTT
GACCCACTTCCTGCAGCCCATTTATCTCAAATCTGTTACATTGGGTTACC
TTTTCAGCCAAGGGCATCTGACCCGGGCTATTTGCTGTCGTGTGACAAGA
GATGGGAGTGCATTTGAGGATGGACTACGACATCCCTTTATTGTCAACCA
CCCCAAGGTTGGCAGAGTCAGCGTATATGATTCCAAAAGGCAATCTGGGA
AGACTAAGGAGACAAGCGTCAACTGGTGTCTGGCTGATGGCTATGACCTG
GAGATCCTGGATGGTACCAGAGGCACCGTGGATGGGCCACGGAATGAATT
GTCCCGGGTCTCCAAAAAGAACATTTTTCTTCTATTTAAGAAGCTCTGCT
CCTTCCGTTACCGCAGGGATCTACTGAGACTCTCCTATGGTGAGGCCAAG
AAAGCTGCCCGTGACTACGAGACGGCCAAGAACTACTTCAAAAAAAGCTT
GAAGGATATGGGCTATGGGAACTGGATTAGCAAACCCCAGGAGGAAAAGA
ACTTTTATCTCTGCCCAGTATAGTATGCTCCGGTGACAGATGGATTAGGG
TGTGTCATACTAGGGTGAGAGAGAGGTAGGTCGTAGCGTTCCTCATCACA
TGGTCAGGGGATTTTTTTTTCCTCCTTTTTTTTCTTTTTAAGCCATAATT
GGTGATACTGAAAACTTTGGGTTCCCATTTATCCTGCTTTCTTTGGGATT
GCTAGGCAAGGTCTGGCCAGGCCCCCCTTTTTTTCCCCCAAGTGAAGAGG
TAGAAACCTAAGAAGTTATCTTTTCTTTCTATAACTCAAAGCATACATAG
TCACTGAGCACCTGCAGTCCATTTCCTCTTAAAAGTTTTTTTTTTTTTCC
ATTTCCTTTCCCTTTGTGTTTGCTACACTGACCTCTTGTGGTCTTGATTA
GGTTTCAGTCAATTCTGGATCATGTCCGAGACTGATGATTTCTTTTGTGG
ATTACGCAGACCCCTCTACTTCCCCCTTCCCCTTCTGAGGTTCTTTCCTT
GTGATCAGAATTTCTCCTTTTCTCCCTCAGAGGGTAAAAAGGTGAACTTA
AAGGATCTGGTGAAACATTTGTAAGGGTAGGAGTTGAAAACTGCAGTTCC
CAGTGTCACGGAAGTGTGATTGGAGCCTACAGATAATGCCCTGCCATCCT
CCCATCCTGCACTTTAGCCAGCTGCAGGGCGGGCAAGGCAAGGAAAGCTG
CTTCCCTGGAAGTGTGCCCCTTTCCCCGGCAGCAGGGAAGTCTAGAACCA
GCCAGACTGGGTTAAGGGAGTTGCCCAAGCCATAGCGGAGGTTTCACCCA
GCAAGGTGACACAGACCACTTCCCAGGAGCACGGGCATGCCTTGGGATAT
TGCCAAGCTTCCAGCTGCCTCTTCTCCTAAAGCACTCCTAGGAATTTGCC
CTGCAAATGCTGGGCGTGCACCCTAGCCAACGTAGGAAAAAATCCAGGAC
AAATCCTAGAGGGTAGAAAATCATCTCTGCTCAGATAATCATGACTTAGC
AAGAATAAGGGCAAAAAATCCTGTTGGCTCACGTCACTGTTCTACCCTGC
ATAATATCTCTCATGACAGTGACACCAAGAGAAGTTGACTAAGTCATATG
TAAATTAGGAGTGTTTAAAAGAATGCCATAGATGTTGATTCTTAACTGCT
ACAGACAACCTATAATTGAGCAGATTTAAAATTCAGGCATACTTTTCCAT
TTATCCAAGTGCTTTCATTTTTCCAGATGGCTTCAGAAGTAGGCTCGTGG
GCAGAGCGCAGACCTGATCTTTATAGTGTTGACATAGAAAGCAGTAGTAG
TGGGTGAAAGGACAGGTTGTCTTCAAACTCTGTGAGGTGGAATCTTTTGT
CTACACCTCCATGAACATTGACTCGTGTGTTCAGAGCCTTTGGCCTCCCT
GTGGAGTCTGGCTCTCTGGCTCCTGTGCATTCTTTGAATAGTCACTCGTA
AAAATTGTCAATGCTTGAAACTATTTCTTTTACTCATGTTGAAGGGACTT
TGTTGACTTTTAGAGTGTTGGTCATGACTCCAAGAGCAGAGCAGGGAAGA
GCCCAAGCATAGACTTGAGTGCCGTGATGGCTGCGGTCCAGTTTTGTGAT
TCTGCTTTTACGTGTTACTTGGTAACAGTCAGCTAGACACACTCAGGAGG
ACTACTGAGGCTCTGCGACCTTCAGGACCTGAGCCTGCCTCTCTCCTTTA
GATGACAGACCTTCATCTGAGAATGTGCTGAGCCAGCACCCTCAGATGAT
TTCCCTCCAAACTGCTGACTAGGTCACCCTCTGTCTGGTAGAGACATTGA
CATCTTTGCTTTTATTCTATACCCTCGGTACTTTTGACCAAAAAATGACC
AAAATAAGAAAATGCAACTTTAAAAAATAGACTAAGGATGCCTTTGCAGA
ACACCAAAGCATCCCAAGGAACCGGTAGGGAAATGATGCCTGTCTCCTGG
GGTAGAAGAGGCCTGCTCCCTGGCTCTGGGTCTGCTGGGGGCACAGTAAA
TCAGTCTTGGCACCCACATCCAGGTCAGAGAGGTCTGTGGTTCTCAGCAT
CAGAAGGCAGCGCAGCCCCTCTCCAGGCTACAGGGTTGTCACCTGCTGAG
TCCTCAAGCTGTTTGGCCTCTCTGGTCCGTCTTGGGCATTAGATTCTCCA
GCAGAGCTCTGGCCAGCTGCCTCCTCTTTAACTGGGAACACGGGCTCTCA
CAAGATCAGAACCCCCACTCACCCCTGAGATCTTATCTAGCAAGCCTGTA
GTATTCCGTTTCTGTTGTAGGAAGAGCGCGAGGCATCCCTGAATTCCATG
CATCTGCTGGAAATGAGCCGTGTCAGATCGCACACCCCCGCGCCCCCATG
CCCCTCTGAGTCACACGGGACAGAGGAGGCAGAGCTTCCGCCCACTGTTA
TCTTCAATTTCTTTGTCCAGTCTTTTGTTTTTAATAAGCAGTGCCCCTCC
CCACTCTTCTTTTTAATGATTTTTGTAGTTGATTTGTCTGAACTGTGGCT
ACTGTGCATTCCTTGAATAATCACTTGTAAAAATTGTCAGTGCTTGAAGC
TGTTTCCTTTACTCACATTGAAGGGACTTTGTTGGTTTTTTGGAGTCTTG
GTTTTGACTCCAAGAACAGAGTCAGGAAGACCCCAAGCATAGACTTGGGT
ACCGTGAAAATGGCTGCAGTCCAGTTTTATGATTCCGCTTTTATGTGTCC
CTTGATAACAGTGACTTAACAATATACATTCCTCATAAATAAAAAAAAAA
CCAAGAATCTGAATTCTTA

SEQ ID NO: 48 - NHP ADAR1 - Transcript Variant X3 Coding Sequence

ATGATGTCACCAATCTGCGACCACACCATTGATTCCCGACTGAAGGGGTA
TTCCCTCAACGGATACTACCCCCATCCATTTCAAGGCTATGAGCACAGAA
AGCTCAGATACCAGCAGCCTGGGCCAGGATCTTCCCCCAATAGTTTCCTG
CTTAAGCAAATAGAGTTTCTCAAGGGGCAGATCCCAGAAGCGCCGGTGAT
TGGAAAGCAGACACCATCACTGCCACCTTCCCTCCCAGGACTCCAGCCAA
GGTTTCCAGTACTACCTGCCTCTAGTACCAGAGGCAGGCAAGTGGATATC
AGGGGTGTCCCCAGGTGCGTGCATCTCGGAAGTCAGGGGCTCCAGAGAGG
GTTCCAGCATCCTTCACCACGTGGCAGGATTCCGCCACGGAGAGGTGTTG
ATTGCCTTTCCTCACAATTCCAGGAACTGAGTATCAACCAAGATCAGGAA
CAAAGGATCTTAAAGTTCTTGGAAGAGCTTGGGGAAGGGAAGGCCACTAC
AGCACATGATCTGTCTTGGAAACTGGGGGCTCCAAAGAAAGAAATCAATC
GAGTTTTATACTCCCTGGCAAAGAAGGGCAAGCTACAGAAAGAGGCAGGA
ACACCCCCTTTGTGGAAAATCGCGGTCTCCACTCAGGCTTGGAACCAGCA
CAGCAGAGTGGTAAGACCAGACGGTCATAGCCAAGGAGCCCCAAACTCAG
ACCCAAATTTGGAACCTGAAGACAGAAACTCCACATCTGTCTCAGAAGAT
CTTGAGCCTTTTATCTCAGTCTCAGCTCAGGCTTGGAACCAGCACAGCGG
AGTGGTAAGACCAGACGGTCATAGCCAAGGAGCCCCAAACTCAGAGTCGA
GTTTGGAACCTGAAGACAGAAACTCCACATCTGCCTTGGAAGATCCTCTT
GAGTTTTTTGACATGGCTGAGATCAAGGAGAAAATCTGCGACTATCTCTT
CAATGTGTCTGACTCCTCTGCCCTGAATTTGGCTAAAAATATTGGCCTCA
CCAAGGCCCGAGATATAAATGCCGTGCTAATTGACATGGAAAGGCAGGGG
GATGTCTATAGACAAGGGACAACCCCTCCCATATGGCATTTGACAGACAA
GAAGCGAGAGAGGATGCAAATCAAGAGAAATACGAACAGTGTTCCTGAAA
CCGCTCCAGCTGCAATCCCTGAGACCAAAAGAAACACAGAGTTCCTCACC
TGTAATATACCCACATCAAATGCCTCAAATAACATAGTAACCACCGAAAA
AGTGGAGAATGGGCAGGAACCTGTCATAAAGTTAGAAAACAGTCAGGAGG
CAAGACCAGAACCAGTAAGACTGAAACCACCTGTTCATAACAATGGCCCC
TCAAAAGCAGGGTATGTTGACTTTGAAAATGGCCAGTGGGCCACAGATGA
CATCCCAGATGACTTGAATAGTATCCGCGCAGCACCAGGTGAGTTTCGAG
CCATCATGGAGATGCCCTCCTTCTACAGTCATGGCTTGCCACGGTGTTCA
CCCTACAAGAAACTGACAGAGTGCCAGCTGAAGAACCCCATCAGCGGGCT
GTTAGAATATGCCCAGTTCGCTAGTCAAACCTGTGAGTTCAACATGATAG
AGCAGAGTGGACCACCCCATGAACCTCGATTTAAATTCCAGGTTGTCATC
AATGGCCGAGAGTTTCCCCCAGCTGAAGCTGGAAGCAAGAAAGTGGCCAA
GCAGGATGCAGCTATGAAAGCCATGACAATTCTGCTAGAGGAAGCCAAAG
CCAAGGACAGTGGAAAATCAGAAGAATCATCCCACTATTCCACAGAAAAA
GAATCAGAGAAGACCACAGAGTCCCAGACCCCCACCCCTTCAGCTACATC
CTTCTTTTCTGGGAAGAGCCCCGTCACCACACTGCTCGAGTGTATGCACA
AATTGGGGAACTCCTGCGAATTCCGTCTCCTGTCCAAAGAAGGCCCTGCC
CATGAGCCCAAGTTCCAGTACTGTGTTGCAGTGGGAGCCCAAACTTTCCC
CAGTGTGAGTGCCCCCAGCAAGAAAGTGGCAAAGCAGATGGCCGCAGAGG
AAGCCATGAAGGCCCTGCATGGGGAGGCAACCAACTCCATGACTTCTGAT
GACCAGCCCGAAGGTATGATCTCAGAGTCACTTGATAACTTGGAATCCGT
GATGCCCAACAAGGTCAGGAAGATTGGCGAGCTCGTGAGATACTTGAACA
CCAATCCTGTGGGTGGCCTTTTGGAGTACGCCCGCTCCCATGGCTTTGCT
GCTGAATTCAAGTTGGTCGACCAGTCTGGACCTCCTCACGAGCCCAAGTT
CGTTTACCAAGCAAAAGTTGGGGGTCGCTGGTTCCCAGCCGTCTGCGCAC
ACAGCAAGAAGCAAGGCAAGCAGGAAGCAGCAGATGCGGCTCTCCGTGTC
TTGATTGGGGAGAACGAGAAGGCAGAACGCATGGGTTTCACAGAGGTAAC
CCCAGTGACAGGGGCCAGTCTCAGAAGAACTATGCTCCTCCTCTCAAGGT
CCCCAGAAGCACAGCCAAAGACACTCCCTCTCACTGGCAGCACCTTCCAT
GACCAGATAGCCATGCTGAGCCACCGGTGCTTCAACACTCTGACTAACAG
CTTCCAGCCTTCCTTGCTCGGCCGCAAGATTCTGGCCGCCATCGTTATGA
AAAAAGACTCTGAGGACATGGGTGTCGTCGTCAGCTTGGGAACAGGGAAT
CGCTGTGTGAAAGGAGATTCTCTCAGCCTGAAGGGAGAAACTGTCAATGA
CTGCCATGCAGAAATCATCTCCCGGAGAGGCTTCATCAGGTTTCTCTACA
GTGAGTTAATGAAATACAACCCCCAGACTGCGAAGGATAGTATATTTGAA
CCTGCTAAGGGAGGAGAAAAGCTCCAAATAAAAAAGACTGTGTCATTCCA
TCTCTATATCAGCACCGCTCCATGTGGAGATGGTGCCCTCTTTGACAAGT
CCTGCAGCGACCGTGCTATGGAAAGCACAGACTCCCGCCACTACCCTGTC
TTTGAGAATCCCAAACAAGGCAAGCTCCGCACCAAGGTGGAAAACGGAGA
AGGCACAATCCCTGTGGAATCCAGTGACATTGTACCTACGTGGGATGGCA
TTCGGCTCGGGGAGAGACTCCGTACCATGTCCTGTAGTGACAAAATCCTA
CGCTGGAATGTGCTGGGCCTGCAAGGGGCACTGTTGACCCACTTCCTGCA
GCCCATTTATCTCAAATCTGTTACATTGGGTTACCTTTTCAGCCAAGGGC
ATCTGACCCGGGCTATTTGCTGTCGTGTGACAAGAGATGGGAGTGCATTT
GAGGATGGACTACGACATCCCTTTATTGTCAACCACCCCAAGGTTGGCAG
AGTCAGCGTATATGATTCCAAAAGGCAATCTGGGAAGACTAAGGAGACAA
GCGTCAACTGGTGTCTGGCTGATGGCTATGACCTGGAGATCCTGGATGGT
ACCAGAGGCACCGTGGATGGGCCACGGAATGAATTGTCCCGGGTCTCCAA
AAAGAACATTTTTCTTCTATTTAAGAAGCTCTGCTCCTTCCGTTACCGCA
GGGATCTACTGAGACTCTCCTATGGTGAGGCCAAGAAAGCTGCCCGTGAC
TACGAGACGGCCAAGAACTACTTCAAAAAAAGCTTGAAGGATATGGGCTA
TGGGAACTGGATTAGCAAACCCCAGGAGGAAAAGAACTTTTATCTCTGCC
CAGTATAG

SEQ ID NO: 49 - NHP ADAR1 - Transcript Variant X3 Amino Acid Sequence

MMSPICDHTIDSRLKGYSLNGYYPHPFQGYEHRKLRYQQPGPGSSPNSFL
LKQIEFLKGQIPEAPVIGKQTPSLPPSLPGLQPRFPVLPASSTRGRQVDI
RGVPRCVHLGSQGLQRGFQHPSPRGRIPPRRGVDCLSSQFQELSINQDQE
QRILKFLEELGEGKATTAHDLSWKLGAPKKEINRVLYSLAKKGKLQKEAG
TPPLWKIAVSTQAWNQHSRVVRPDGHSQGAPNSDPNLEPEDRNSTSVSED
LEPFISVSAQAWNQHSGVVRPDGHSQGAPNSESSLEPEDRNSTSALEDPL
EFFDMAEIKEKICDYLFNVSDSSALNLAKNIGLTKARDINAVLIDMERQG
DVYRQGTTPPIWHLTDKKRERMQIKRNTNSVPETAPAAIPETKRNTEFLT
CNIPTSNASNNIVTTEKVENGQEPVIKLENSQEARPEPVRLKPPVHNNGP
SKAGYVDFENGQWATDDIPDDLNSIRAAPGEFRAIMEMPSFYSHGLPRCS
PYKKLTECQLKNPISGLLEYAQFASQTCEFNMIEQSGPPHEPRFKFQVVI
NGREFPPAEAGSKKVAKQDAAMKAMTILLEEAKAKDSGKSEESSHYSTEK
ESEKTTESQTPTPSATSFFSGKSPVTTLLECMHKLGNSCEFRLLSKEGPA
HEPKFQYCVAVGAQTFPSVSAPSKKVAKQMAAEEAMKALHGEATNSMTSD
DQPEGMISESLDNLESVMPNKVRKIGELVRYLNTNPVGGLLEYARSHGFA
AEFKLVDQSGPPHEPKFVYQAKVGGRWFPAVCAHSKKQGKQEAADAALRV
LIGENEKAERMGFTEVTPVTGASLRRTMLLLSRSPEAQPKTLPLTGSTFH
DQIAMLSHRCFNTLTNSFQPSLLGRKILAAIVMKKDSEDMGVVVSLGTGN
RCVKGDSLSLKGETVNDCHAEIISRRGFIRFLYSELMKYNPQTAKDSIFE
PAKGGEKLQIKKTVSFHLYISTAPCGDGALFDKSCSDRAMESTDSRHYPV
FENPKQGKLRTKVENGEGTIPVESSDIVPTWDGIRLGERLRTMSCSDKIL
RWNVLGLQGALLTHFLQPIYLKSVTLGYLFSQGHLTRAICCRVTRDGSAF
EDGLRHPFIVNHPKVGRVSVYDSKRQSGKTKETSVNWCLADGYDLEILDG
TRGTVDGPRNELSRVSKKNIFLLFKKLCSFRYRRDLLRLSYGEAKKAARD
YETAKNYFKKSLKDMGYGNWISKPQEEKNFYLCPV

In certain embodiments, an ADAR1 polynucleotide or an ADAR1 gene incorporated into a non-human animal (e.g., a rodent, e.g., a rat or mouse) is represented by or comprises a sequence encoding a non-human primate (NHP) ADAR1 transcript. In some embodiments, a NHP ADAR1 transcript variant is predicted transcript variant X4. In certain embodiments, an ADAR1 polynucleotide or a NHP ADAR1 gene comprises, or consists of, a nucleotide sequence that is the same as, differs by no more than 1, 2, 3, 4, 5, 6, 7, 8, 9, or 10 nucleotides from, or has at least has at least 90%, 92%, 94%, 96%, 98%, or 99% homology with, the nucleotide sequence of SEQ ID NO: 50 or SEQ ID NO: 51 or a characteristic portion thereof. In some embodiments, an ADAR1 polynucleotide or a NHP ADAR1 gene comprises, or consists of, a nucleotide sequence that is the same as the nucleotide sequence of SEQ ID NO: 50 or SEQ ID NO: 51 or a characteristic portion thereof. In certain embodiments, an encoded ADAR1 Amino Acid sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the Amino Acid sequence of SEQ ID NO: 52 or a characteristic portion thereof. In certain embodiments, the amino acid sequence of an encoded and/or expressed ADAR1 polypeptide or a characteristic portion thereof, is or comprises a sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the amino acid sequence of SEQ ID NO: 52 or a characteristic portion thereof.

SEQ ID NO: 50 - NHP ADAR1 - Transcript Variant X4 cDNA Sequence

TCCTCTTGGCCAAACTTTCCGGAGGGGAAGGCTTTCAGAGGAAACGAAAG
CGAAATTGAACCGGAGCCATCTTGGGCCCGGCGCGCAGACCCGCAGAGTT
TCCTGTGCCGACGCCTCGGGCTACTTGCAGTGCGAAGCAGCAGGGGTGTG
GGGGCCTCGAGGGGCTGGCGCGGCCCAGCGGTCTGGCCAGGGTCGTGCCG
CCGGCGGGTCGGGTTGGGTCGGGCAATGCCTCTCCGGCGCCATGAACCCG
CGGCAGGGGTATTCCCTCAACGGATACTACCCCCATCCATTTCAAGGCTA
TGAGCACAGAAAGCTCAGATACCAGCAGCCTGGGCCAGGATCTTCCCCCA
ATAGTTTCCTGCTTAAGCAAATAGAGTTTCTCAAGGGGCAGATCCCAGAA
GCGCCGGTGATTGGAAAGCAGACACCATCACTGCCACCTTCCCTCCCAGG
ACTCCAGCCAAGGTTTCCAGTACTACCTGCCTCTAGTACCAGAGGCAGGC
AAGTGGATATCAGGGGTGTCCCCAGGTGCGTGCATCTCGGAAGTCAGGGG
CTCCAGAGAGGGTTCCAGCATCCTTCACCACGTGGCAGGATTCCGCCACG
GAGAGGTGTTGATTGCCTTTCCTCACAATTCCAGGAACTGAGTATCAACC
AAGATCAGGAACAAAGGATCTTAAAGTTCTTGGAAGAGCTTGGGGAAGGG
AAGGCCACTACAGCACATGATCTGTCTTGGAAACTGGGGGCTCCAAAGAA
AGAAATCAATCGAGTTTTATACTCCCTGGCAAAGAAGGGCAAGCTACAGA
AAGAGGCAGGAACACCCCCTTTGTGGAAAATCGCGGTCTCCACTCAGGCT
TGGAACCAGCACAGCAGAGTGGTAAGACCAGACGGTCATAGCCAAGGAGC
CCCAAACTCAGACCCAAATTTGGAACCTGAAGACAGAAACTCCACATCTG
TCTCAGAAGATCTTGAGCCTTTTATCTCAGTCTCAGCTCAGGCTTGGAAC
CAGCACAGCGGAGTGGTAAGACCAGACGGTCATAGCCAAGGAGCCCCAAA
CTCAGAGTCGAGTTTGGAACCTGAAGACAGAAACTCCACATCTGCCTTGG
AAGATCCTCTTGAGTTTTTTGACATGGCTGAGATCAAGGAGAAAATCTGC
GACTATCTCTTCAATGTGTCTGACTCCTCTGCCCTGAATTTGGCTAAAAA
TATTGGCCTCACCAAGGCCCGAGATATAAATGCCGTGCTAATTGACATGG
AAAGGCAGGGGGATGTCTATAGACAAGGGACAACCCCTCCCATATGGCAT
TTGACAGACAAGAAGCGAGAGAGGATGCAAATCAAGAGAAATACGAACAG
TGTTCCTGAAACCGCTCCAGCTGCAATCCCTGAGACCAAAAGAAACACAG
AGTTCCTCACCTGTAATATACCCACATCAAATGCCTCAAATAACATAGTA
ACCACCGAAAAAGTGGAGAATGGGCAGGAACCTGTCATAAAGTTAGAAAA
CAGTCAGGAGGCAAGACCAGAACCAGTAAGACTGAAACCACCTGTTCATA
ACAATGGCCCCTCAAAAGCAGGGTATGTTGACTTTGAAAATGGCCAGTGG
GCCACAGATGACATCCCAGATGACTTGAATAGTATCCGCGCAGCACCAGG
TGAGTTTCGAGCCATCATGGAGATGCCCTCCTTCTACAGTCATGGCTTGC
CACGGTGTTCACCCTACAAGAAACTGACAGAGTGCCAGCTGAAGAACCCC
ATCAGCGGGCTGTTAGAATATGCCCAGTTCGCTAGTCAAACCTGTGAGTT
CAACATGATAGAGCAGAGTGGACCACCCCATGAACCTCGATTTAAATTCC
AGGTTGTCATCAATGGCCGAGAGTTTCCCCCAGCTGAAGCTGGAAGCAAG
AAAGTGGCCAAGCAGGATGCAGCTATGAAAGCCATGACAATTCTGCTAGA
GGAAGCCAAAGCCAAGGACAGTGGAAAATCAGAAGAATCATCCCACTATT
CCACAGAAAAAGAATCAGAGAAGACCACAGAGTCCCAGACCCCCACCCCT
TCAGCTACATCCTTCTTTTCTGGGAAGAGCCCCGTCACCACACTGCTCGA
GTGTATGCACAAATTGGGGAACTCCTGCGAATTCCGTCTCCTGTCCAAAG
AAGGCCCTGCCCATGAGCCCAAGTTCCAGTACTGTGTTGCAGTGGGAGCC
CAAACTTTCCCCAGTGTGAGTGCCCCCAGCAAGAAAGTGGCAAAGCAGAT
GGCCGCAGAGGAAGCCATGAAGGCCCTGCATGGGGAGGCAACCAACTCCA
TGACTTCTGATGACCAGCCCGAAGGTATGATCTCAGAGTCACTTGATAAC
TTGGAATCCGTGATGCCCAACAAGGTCAGGAAGATTGGCGAGCTCGTGAG
ATACTTGAACACCAATCCTGTGGGTGGCCTTTTGGAGTACGCCCGCTCCC
ATGGCTTTGCTGCTGAATTCAAGTTGGTCGACCAGTCTGGACCTCCTCAC
GAGCCCAAGTTCGTTTACCAAGCAAAAGTTGGGGGTCGCTGGTTCCCAGC
CGTCTGCGCACACAGCAAGAAGCAAGGCAAGCAGGAAGCAGCAGATGCGG
CTCTCCGTGTCTTGATTGGGGAGAACGAGAAGGCAGAACGCATGGGTTTC
ACAGAGGTAACCCCAGTGACAGGGGCCAGTCTCAGAAGAACTATGCTCCT
CCTCTCAAGGTCCCCAGAAGCACAGCCAAAGACACTCCCTCTCACTGGCA
GCACCTTCCATGACCAGATAGCCATGCTGAGCCACCGGTGCTTCAACACT
CTGACTAACAGCTTCCAGCCTTCCTTGCTCGGCCGCAAGATTCTGGCCGC
CATCGTTATGAAAAAAGACTCTGAGGACATGGGTGTCGTCGTCAGCTTGG
GAACAGGGAATCGCTGTGTGAAAGGAGATTCTCTCAGCCTGAAGGGAGAA
ACTGTCAATGACTGCCATGCAGAAATCATCTCCCGGAGAGGCTTCATCAG
GTTTCTCTACAGTGAGTTAATGAAATACAACCCCCAGACTGCGAAGGATA
GTATATTTGAACCTGCTAAGGGAGGAGAAAAGCTCCAAATAAAAAAGACT
GTGTCATTCCATCTCTATATCAGCACCGCTCCATGTGGAGATGGTGCCCT
CTTTGACAAGTCCTGCAGCGACCGTGCTATGGAAAGCACAGACTCCCGCC
ACTACCCTGTCTTTGAGAATCCCAAACAAGGCAAGCTCCGCACCAAGGTG
GAAAACGGAGAAGGCACAATCCCTGTGGAATCCAGTGACATTGTACCTAC
GTGGGATGGCATTCGGCTCGGGGAGAGACTCCGTACCATGTCCTGTAGTG
ACAAAATCCTACGCTGGAATGTGCTGGGCCTGCAAGGGGCACTGTTGACC
CACTTCCTGCAGCCCATTTATCTCAAATCTGTTACATTGGGTTACCTTTT
CAGCCAAGGGCATCTGACCCGGGCTATTTGCTGTCGTGTGACAAGAGATG
GGAGTGCATTTGAGGATGGACTACGACATCCCTTTATTGTCAACCACCCC
AAGGTTGGCAGAGTCAGCGTATATGATTCCAAAAGGCAATCTGGGAAGAC
TAAGGAGACAAGCGTCAACTGGTGTCTGGCTGATGGCTATGACCTGGAGA
TCCTGGATGGTACCAGAGGCACCGTGGATGGGCCACGGAATGAATTGTCC
CGGGTCTCCAAAAAGAACATTTTTCTTCTATTTAAGAAGCTCTGCTCCTT
CCGTTACCGCAGGGATCTACTGAGACTCTCCTATGGTGAGGCCAAGAAAG
CTGCCCGTGACTACGAGACGGCCAAGAACTACTTCAAAAAAAGCTTGAAG
GATATGGGCTATGGGAACTGGATTAGCAAACCCCAGGAGGAAAAGAACTT
TTATCTCTGCCCAGTATAGTATGCTCCGGTGACAGATGGATTAGGGTGTG
TCATACTAGGGTGAGAGAGAGGTAGGTCGTAGCGTTCCTCATCACATGGT
CAGGGGATTTTTTTTTCCTCCTTTTTTTTCTTTTTAAGCCATAATTGGTG
ATACTGAAAACTTTGGGTTCCCATTTATCCTGCTTTCTTTGGGATTGCTA
GGCAAGGTCTGGCCAGGCCCCCCTTTTTTTCCCCCAAGTGAAGAGGTAGA
AACCTAAGAAGTTATCTTTTCTTTCTATAACTCAAAGCATACATAGTCAC
TGAGCACCTGCAGTCCATTTCCTCTTAAAAGTTTTTTTTTTTTTCCATTT
CCTTTCCCTTTGTGTTTGCTACACTGACCTCTTGTGGTCTTGATTAGGTT
TCAGTCAATTCTGGATCATGTCCGAGACTGATGATTTCTTTTGTGGATTA
CGCAGACCCCTCTACTTCCCCCTTCCCCTTCTGAGGTTCTTTCCTTGTGA
TCAGAATTTCTCCTTTTCTCCCTCAGAGGGTAAAAAGGTGAACTTAAAGG
ATCTGGTGAAACATTTGTAAGGGTAGGAGTTGAAAACTGCAGTTCCCAGT
GTCACGGAAGTGTGATTGGAGCCTACAGATAATGCCCTGCCATCCTCCCA
TCCTGCACTTTAGCCAGCTGCAGGGCGGGCAAGGCAAGGAAAGCTGCTTC
CCTGGAAGTGTGCCCCTTTCCCCGGCAGCAGGGAAGTCTAGAACCAGCCA
GACTGGGTTAAGGGAGTTGCCCAAGCCATAGCGGAGGTTTCACCCAGCAA
GGTGACACAGACCACTTCCCAGGAGCACGGGCATGCCTTGGGATATTGCC
AAGCTTCCAGCTGCCTCTTCTCCTAAAGCACTCCTAGGAATTTGCCCTGC
AAATGCTGGGCGTGCACCCTAGCCAACGTAGGAAAAAATCCAGGACAAAT
CCTAGAGGGTAGAAAATCATCTCTGCTCAGATAATCATGACTTAGCAAGA
ATAAGGGCAAAAAATCCTGTTGGCTCACGTCACTGTTCTACCCTGCATAA
TATCTCTCATGACAGTGACACCAAGAGAAGTTGACTAAGTCATATGTAAA
TTAGGAGTGTTTAAAAGAATGCCATAGATGTTGATTCTTAACTGCTACAG
ACAACCTATAATTGAGCAGATTTAAAATTCAGGCATACTTTTCCATTTAT
CCAAGTGCTTTCATTTTTCCAGATGGCTTCAGAAGTAGGCTCGTGGGCAG
AGCGCAGACCTGATCTTTATAGTGTTGACATAGAAAGCAGTAGTAGTGGG
TGAAAGGACAGGTTGTCTTCAAACTCTGTGAGGTGGAATCTTTTGTCTAC
ACCTCCATGAACATTGACTCGTGTGTTCAGAGCCTTTGGCCTCCCTGTGG
AGTCTGGCTCTCTGGCTCCTGTGCATTCTTTGAATAGTCACTCGTAAAAA
TTGTCAATGCTTGAAACTATTTCTTTTACTCATGTTGAAGGGACTTTGTT
GACTTTTAGAGTGTTGGTCATGACTCCAAGAGCAGAGCAGGGAAGAGCCC
AAGCATAGACTTGAGTGCCGTGATGGCTGCGGTCCAGTTTTGTGATTCTG
CTTTTACGTGTTACTTGGTAACAGTCAGCTAGACACACTCAGGAGGACTA
CTGAGGCTCTGCGACCTTCAGGACCTGAGCCTGCCTCTCTCCTTTAGATG
ACAGACCTTCATCTGAGAATGTGCTGAGCCAGCACCCTCAGATGATTTCC
CTCCAAACTGCTGACTAGGTCACCCTCTGTCTGGTAGAGACATTGACATC
TTTGCTTTTATTCTATACCCTCGGTACTTTTGACCAAAAAATGACCAAAA
TAAGAAAATGCAACTTTAAAAAATAGACTAAGGATGCCTTTGCAGAACAC
CAAAGCATCCCAAGGAACCGGTAGGGAAATGATGCCTGTCTCCTGGGGTA
GAAGAGGCCTGCTCCCTGGCTCTGGGTCTGCTGGGGGCACAGTAAATCAG
TCTTGGCACCCACATCCAGGTCAGAGAGGTCTGTGGTTCTCAGCATCAGA
AGGCAGCGCAGCCCCTCTCCAGGCTACAGGGTTGTCACCTGCTGAGTCCT
CAAGCTGTTTGGCCTCTCTGGTCCGTCTTGGGCATTAGATTCTCCAGCAG
AGCTCTGGCCAGCTGCCTCCTCTTTAACTGGGAACACGGGCTCTCACAAG
ATCAGAACCCCCACTCACCCCTGAGATCTTATCTAGCAAGCCTGTAGTAT
TCCGTTTCTGTTGTAGGAAGAGCGCGAGGCATCCCTGAATTCCATGCATC
TGCTGGAAATGAGCCGTGTCAGATCGCACACCCCCGCGCCCCCATGCCCC
TCTGAGTCACACGGGACAGAGGAGGCAGAGCTTCCGCCCACTGTTATCTT
CAATTTCTTTGTCCAGTCTTTTGTTTTTAATAAGCAGTGCCCCTCCCCAC
TCTTCTTTTTAATGATTTTTGTAGTTGATTTGTCTGAACTGTGGCTACTG
TGCATTCCTTGAATAATCACTTGTAAAAATTGTCAGTGCTTGAAGCTGTT
TCCTTTACTCACATTGAAGGGACTTTGTTGGTTTTTTGGAGTCTTGGTTT
TGACTCCAAGAACAGAGTCAGGAAGACCCCAAGCATAGACTTGGGTACCG
TGAAAATGGCTGCAGTCCAGTTTTATGATTCCGCTTTTATGTGTCCCTTG
ATAACAGTGACTTAACAATATACATTCCTCATAAATAAAAAAAAAACCAA
GAATCTGAATTCTTA

SEQ ID NO: 51 - NHP ADAR1 - Transcript Variant X4 Coding Sequence

ATGAACCCGCGGCAGGGGTATTCCCTCAACGGATACTACCCCCATCCATT
TCAAGGCTATGAGCACAGAAAGCTCAGATACCAGCAGCCTGGGCCAGGAT
CTTCCCCCAATAGTTTCCTGCTTAAGCAAATAGAGTTTCTCAAGGGGCAG
ATCCCAGAAGCGCCGGTGATTGGAAAGCAGACACCATCACTGCCACCTTC
CCTCCCAGGACTCCAGCCAAGGTTTCCAGTACTACCTGCCTCTAGTACCA
GAGGCAGGCAAGTGGATATCAGGGGTGTCCCCAGGTGCGTGCATCTCGGA
AGTCAGGGGCTCCAGAGAGGGTTCCAGCATCCTTCACCACGTGGCAGGAT
TCCGCCACGGAGAGGTGTTGATTGCCTTTCCTCACAATTCCAGGAACTGA
GTATCAACCAAGATCAGGAACAAAGGATCTTAAAGTTCTTGGAAGAGCTT
GGGGAAGGGAAGGCCACTACAGCACATGATCTGTCTTGGAAACTGGGGGC
TCCAAAGAAAGAAATCAATCGAGTTTTATACTCCCTGGCAAAGAAGGGCA
AGCTACAGAAAGAGGCAGGAACACCCCCTTTGTGGAAAATCGCGGTCTCC
ACTCAGGCTTGGAACCAGCACAGCAGAGTGGTAAGACCAGACGGTCATAG
CCAAGGAGCCCCAAACTCAGACCCAAATTTGGAACCTGAAGACAGAAACT
CCACATCTGTCTCAGAAGATCTTGAGCCTTTTATCTCAGTCTCAGCTCAG
GCTTGGAACCAGCACAGCGGAGTGGTAAGACCAGACGGTCATAGCCAAGG
AGCCCCAAACTCAGAGTCGAGTTTGGAACCTGAAGACAGAAACTCCACAT
CTGCCTTGGAAGATCCTCTTGAGTTTTTTGACATGGCTGAGATCAAGGAG
AAAATCTGCGACTATCTCTTCAATGTGTCTGACTCCTCTGCCCTGAATTT
GGCTAAAAATATTGGCCTCACCAAGGCCCGAGATATAAATGCCGTGCTAA
TTGACATGGAAAGGCAGGGGGATGTCTATAGACAAGGGACAACCCCTCCC
ATATGGCATTTGACAGACAAGAAGCGAGAGAGGATGCAAATCAAGAGAAA
TACGAACAGTGTTCCTGAAACCGCTCCAGCTGCAATCCCTGAGACCAAAA
GAAACACAGAGTTCCTCACCTGTAATATACCCACATCAAATGCCTCAAAT
AACATAGTAACCACCGAAAAAGTGGAGAATGGGCAGGAACCTGTCATAAA
GTTAGAAAACAGTCAGGAGGCAAGACCAGAACCAGTAAGACTGAAACCAC
CTGTTCATAACAATGGCCCCTCAAAAGCAGGGTATGTTGACTTTGAAAAT
GGCCAGTGGGCCACAGATGACATCCCAGATGACTTGAATAGTATCCGCGC
AGCACCAGGTGAGTTTCGAGCCATCATGGAGATGCCCTCCTTCTACAGTC
ATGGCTTGCCACGGTGTTCACCCTACAAGAAACTGACAGAGTGCCAGCTG
AAGAACCCCATCAGCGGGCTGTTAGAATATGCCCAGTTCGCTAGTCAAAC
CTGTGAGTTCAACATGATAGAGCAGAGTGGACCACCCCATGAACCTCGAT
TTAAATTCCAGGTTGTCATCAATGGCCGAGAGTTTCCCCCAGCTGAAGCT
GGAAGCAAGAAAGTGGCCAAGCAGGATGCAGCTATGAAAGCCATGACAAT
TCTGCTAGAGGAAGCCAAAGCCAAGGACAGTGGAAAATCAGAAGAATCAT
CCCACTATTCCACAGAAAAAGAATCAGAGAAGACCACAGAGTCCCAGACC
CCCACCCCTTCAGCTACATCCTTCTTTTCTGGGAAGAGCCCCGTCACCAC
ACTGCTCGAGTGTATGCACAAATTGGGGAACTCCTGCGAATTCCGTCTCC
TGTCCAAAGAAGGCCCTGCCCATGAGCCCAAGTTCCAGTACTGTGTTGCA
GTGGGAGCCCAAACTTTCCCCAGTGTGAGTGCCCCCAGCAAGAAAGTGGC
AAAGCAGATGGCCGCAGAGGAAGCCATGAAGGCCCTGCATGGGGAGGCAA
CCAACTCCATGACTTCTGATGACCAGCCCGAAGGTATGATCTCAGAGTCA
CTTGATAACTTGGAATCCGTGATGCCCAACAAGGTCAGGAAGATTGGCGA
GCTCGTGAGATACTTGAACACCAATCCTGTGGGTGGCCTTTTGGAGTACG
CCCGCTCCCATGGCTTTGCTGCTGAATTCAAGTTGGTCGACCAGTCTGGA
CCTCCTCACGAGCCCAAGTTCGTTTACCAAGCAAAAGTTGGGGGTCGCTG
GTTCCCAGCCGTCTGCGCACACAGCAAGAAGCAAGGCAAGCAGGAAGCAG
CAGATGCGGCTCTCCGTGTCTTGATTGGGGAGAACGAGAAGGCAGAACGC
ATGGGTTTCACAGAGGTAACCCCAGTGACAGGGGCCAGTCTCAGAAGAAC
TATGCTCCTCCTCTCAAGGTCCCCAGAAGCACAGCCAAAGACACTCCCTC
TCACTGGCAGCACCTTCCATGACCAGATAGCCATGCTGAGCCACCGGTGC
TTCAACACTCTGACTAACAGCTTCCAGCCTTCCTTGCTCGGCCGCAAGAT
TCTGGCCGCCATCGTTATGAAAAAAGACTCTGAGGACATGGGTGTCGTCG
TCAGCTTGGGAACAGGGAATCGCTGTGTGAAAGGAGATTCTCTCAGCCTG
AAGGGAGAAACTGTCAATGACTGCCATGCAGAAATCATCTCCCGGAGAGG
CTTCATCAGGTTTCTCTACAGTGAGTTAATGAAATACAACCCCCAGACTG
CGAAGGATAGTATATTTGAACCTGCTAAGGGAGGAGAAAAGCTCCAAATA
AAAAAGACTGTGTCATTCCATCTCTATATCAGCACCGCTCCATGTGGAGA
TGGTGCCCTCTTTGACAAGTCCTGCAGCGACCGTGCTATGGAAAGCACAG
ACTCCCGCCACTACCCTGTCTTTGAGAATCCCAAACAAGGCAAGCTCCGC
ACCAAGGTGGAAAACGGAGAAGGCACAATCCCTGTGGAATCCAGTGACAT
TGTACCTACGTGGGATGGCATTCGGCTCGGGGAGAGACTCCGTACCATGT
CCTGTAGTGACAAAATCCTACGCTGGAATGTGCTGGGCCTGCAAGGGGCA
CTGTTGACCCACTTCCTGCAGCCCATTTATCTCAAATCTGTTACATTGGG
TTACCTTTTCAGCCAAGGGCATCTGACCCGGGCTATTTGCTGTCGTGTGA
CAAGAGATGGGAGTGCATTTGAGGATGGACTACGACATCCCTTTATTGTC
AACCACCCCAAGGTTGGCAGAGTCAGCGTATATGATTCCAAAAGGCAATC
TGGGAAGACTAAGGAGACAAGCGTCAACTGGTGTCTGGCTGATGGCTATG
ACCTGGAGATCCTGGATGGTACCAGAGGCACCGTGGATGGGCCACGGAAT
GAATTGTCCCGGGTCTCCAAAAAGAACATTTTTCTTCTATTTAAGAAGCT
CTGCTCCTTCCGTTACCGCAGGGATCTACTGAGACTCTCCTATGGTGAGG
CCAAGAAAGCTGCCCGTGACTACGAGACGGCCAAGAACTACTTCAAAAAA
AGCTTGAAGGATATGGGCTATGGGAACTGGATTAGCAAACCCCAGGAGGA
AAAGAACTTTTATCTCTGCCCAGTATAG

SEQ ID NO: 52 - NHP ADAR1 - Transcript Variant X4 Amino Acid Sequence

NPRQGYSLNGYYPHPFQGYEHRKLRYQQPGPGSSPNSFLLKQIEFLKGQI
PEAPVIGKQTPSLPPSLPGLQPRFPVLPASSTRGRQVDIRGVPRCVHLGS
QGLQRGFQHPSPRGRIPPRRGVDCLSSQFQELSINQDQEQRILKFLEELG
EGKATTAHDLSWKLGAPKKEINRVLYSLAKKGKLQKEAGTPPLWKIAVST
QAWNQHSRVVRPDGHSQGAPNSDPNLEPEDRNSTSVSEDLEPFISVSAQA
WNQHSGVVRPDGHSQGAPNSESSLEPEDRNSTSALEDPLEFFDMAEIKEK
ICDYLFNVSDSSALNLAKNIGLTKARDINAVLIDMERQGDVYRQGTTPPI
WHLTDKKRERMQIKRNTNSVPETAPAAIPETKRNTEFLTCNIPTSNASNN
IVTTEKVENGQEPVIKLENSQEARPEPVRLKPPVHNNGPSKAGYVDFENG
QWATDDIPDDLNSIRAAPGEFRAIMEMPSFYSHGLPRCSPYKKLTECQLK
NPISGLLEYAQFASQTCEFNMIEQSGPPHEPRFKFQVVINGREFPPAEAG
SKKVAKQDAAMKAMTILLEEAKAKDSGKSEESSHYSTEKESEKTTESQTP
TPSATSFFSGKSPVTTLLECMHKLGNSCEFRLLSKEGPAHEPKFQYCVAV
GAQTFPSVSAPSKKVAKQMAAEEAMKALHGEATNSMTSDDQPEGMISESL
DNLESVMPNKVRKIGELVRYLNTNPVGGLLEYARSHGFAAEFKLVDQSGP
PHEPKFVYQAKVGGRWFPAVCAHSKKQGKQEAADAALRVLIGENEKAERM
GFTEVTPVTGASLRRTMLLLSRSPEAQPKTLPLTGSTFHDQIAMLSHRCF
NTLTNSFQPSLLGRKILAAIVMKKDSEDMGVVVSLGTGNRCVKGDSLSLK
GETVNDCHAEIISRRGFIRFLYSELMKYNPQTAKDSIFEPAKGGEKLQIK
KTVSFHLYISTAPCGDGALFDKSCSDRAMESTDSRHYPVFENPKQGKLRT
KVENGEGTIPVESSDIVPTWDGIRLGERLRTMSCSDKILRWNVLGLQGAL
LTHFLQPIYLKSVTLGYLFSQGHLTRAICCRVTRDGSAFEDGLRHPFIVN
HPKVGRVSVYDSKRQSGKTKETSVNWCLADGYDLEILDGTRGTVDGPRNE
LSRVSKKNIFLLFKKLCSFRYRRDLLRLSYGEAKKAARDYETAKNYFKKS
LKDMGYGNWISKPQEEKNFYLCPV

In certain embodiments, an ADAR1 polynucleotide or an ADAR1 gene incorporated into a non-human animal (e.g., a rodent, e.g., a rat or mouse) is represented by or comprises a sequence encoding a non-human primate (NHP) ADAR1 transcript variant. In some embodiments, a NHP ADAR1 transcript variant is predicted transcript variant X5. In certain embodiments, an ADAR1 polynucleotide or a NHP ADAR1 gene comprises, or consists of, a nucleotide sequence that is the same as, differs by no more than 1, 2, 3, 4, 5, 6, 7, 8, 9, or 10 nucleotides from, or has at least has at least 90%, 92%, 94%, 96%, 98%, or 99% homology with, the nucleotide sequence of SEQ ID NO: 53 or SEQ ID NO: 54 or a characteristic portion thereof. In some embodiments, an ADAR1 polynucleotide or a NHP ADAR1 gene comprises, or consists of, a nucleotide sequence that is the same as the nucleotide sequence of SEQ ID NO: 53 or SEQ ID NO: 54 or a characteristic portion thereof. In certain embodiments, an encoded ADAR1 Amino Acid sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the Amino Acid sequence of SEQ ID NO: 55 or a characteristic portion thereof. In certain embodiments, the amino acid sequence of an encoded and/or expressed ADAR1 polypeptide or a characteristic portion thereof, is or comprises a sequence that is the same as, differs by no more than 1, 2, 3, 4, 5, 6, 7, 8, 9, or 10 Amino Acids from, or has at least has at least 90%, 92%, 94%, 96%, 98%, or 99% homology with, the amino acid sequence of SEQ ID NO: 55 or a characteristic portion thereof.

SEQ ID NO: 53 - NHP ADAR1 - Transcript Variant X5 cDNA Sequence

CCTCTTGGCCAAACTTTCCGGAGGGGAAGGCTTTCAGAGGAAACGAAAGC
GAAATTGAACCGGAGCCATCTTGGGCCCGGCGCGCAGACCCGCAGAGTTT
CCTGTGCCGACGCCTCGGGCTACTTGCAGTGCGAAGCAGCAGGGGTGTGG
GGGCCTCGAGGGGCTGGCGCGGCCCAGCGGTCTGGCCAGGGTCGTGCCGC
CGGCGGGTCGGGTTGGGTCGGGCAATGCCTCTCCGGCGCCATGAACCCGC
GGCAGGGGTATTCCCTCAACGGATACTACCCCCATCCATTTCAAGGCTAT
GAGCACAGAAAGCTCAGATACCAGCAGCCTGGGCCAGGATCTTCCCCCAA
TAGTTTCCTGCTTAAGCAAATAGAGTTTCTCAAGGGGCAGATCCCAGAAG
CGCCGGTGATTGGAAAGCAGACACCATCACTGCCACCTTCCCTCCCAGGA
CTCCAGCCAAGGTTTCCAGTACTACCTGCCTCTAGTACCAGAGGCAGGCA
AGTGGATATCAGGGGTGTCCCCAGGTGCGTGCATCTCGGAAGTCAGGGGC
TCCAGAGAGGGTTCCAGCATCCTTCACCACGTGGCAGGATTCCGCCACGG
AGAGGTGTTGATTGCCTTTCCTCACAATTCCAGGAACTGAGTATCAACCA
AGATCAGGAACAAAGGATCTTAAAGTTCTTGGAAGAGCTTGGGGAAGGGA
AGGCCACTACAGCACATGATCTGTCTTGGAAACTGGGGGCTCCAAAGAAA
GAAATCAATCGAGTTTTATACTCCCTGGCAAAGAAGGGCAAGCTACAGAA
AGAGGCAGGAACACCCCCTTTGTGGAAAATCGCGGTCTCCACTCAGGCTT
GGAACCAGCACAGCAGAGTGGTAAGACCAGACGGTCATAGCCAAGGAGCC
CCAAACTCAGACCCAAATTTGGAACCTGAAGACAGAAACTCCACATCTGT
CTCAGAAGATCTTGAGCCTTTTATCTCAGTCTCAGCTCAGGCTTGGAACC
AGCACAGCGGAGTGGTAAGACCAGACGGTCATAGCCAAGGAGCCCCAAAC
TCAGAGTCGAGTTTGGAACCTGAAGACAGAAACTCCACATCTGCCTTGGA
AGATCCTCTTGAGTTTTTTGACATGGCTGAGATCAAGGAGAAAATCTGCG
ACTATCTCTTCAATGTGTCTGACTCCTCTGCCCTGAATTTGGCTAAAAAT
ATTGGCCTCACCAAGGCCCGAGATATAAATGCCGTGCTAATTGACATGGA
AAGGCAGGGGGATGTCTATAGACAAGGGACAACCCCTCCCATATGGCATT
TGACAGACAAGAAGCGAGAGAGGATGCAAATCAAGAGAAATACGAACAGT
GTTCCTGAAACCGCTCCAGCTGCAATCCCTGAGACCAAAAGAAACACAGA
GTTCCTCACCTGTAATATACCCACATCAAATGCCTCAAATAACATAGTAA
CCACCGAAAAAGTGGAGAATGGGCAGGAACCTGTCATAAAGTTAGAAAAC
AGTCAGGAGGCAAGACCAGAACCAGTAAGACTGAAACCACCTGTTCATAA
CAATGGCCCCTCAAAAGCAGGGTATGTTGACTTTGAAAATGGCCAGTGGG
CCACAGATGACATCCCAGATGACTTGAATAGTATCCGCGCAGCACCAGGT
GAGTTTCGAGCCATCATGGAGATGCCCTCCTTCTACAGTCATGGCTTGCC
ACGGTGTTCACCCTACAAGAAACTGACAGAGTGCCAGCTGAAGAACCCCA
TCAGCGGGCTGTTAGAATATGCCCAGTTCGCTAGTCAAACCTGTGAGTTC
AACATGATAGAGCAGAGTGGACCACCCCATGAACCTCGATTTAAATTCCA
GGTTGTCATCAATGGCCGAGAGTTTCCCCCAGCTGAAGCTGGAAGCAAGA
AAGTGGCCAAGCAGGATGCAGCTATGAAAGCCATGACAATTCTGCTAGAG
GAAGCCAAAGCCAAGGACAGTGGAAAATCAGAAGAATCATCCCACTATTC
CACAGAAAAAGAATCAGAGAAGACCACAGAGTCCCAGACCCCCACCCCTT
CAGCTACATCCTTCTTTTCTGGGAAGAGCCCCGTCACCACACTGCTCGAG
TGTATGCACAAATTGGGGAACTCCTGCGAATTCCGTCTCCTGTCCAAAGA
AGGCCCTGCCCATGAGCCCAAGTTCCAGTACTGTGTTGCAGTGGGAGCCC
AAACTTTCCCCAGTGTGAGTGCCCCCAGCAAGAAAGTGGCAAAGCAGATG
GCCGCAGAGGAAGCCATGAAGGCCCTGCATGGGGAGGCAACCAACTCCAT
GACTTCTGATGACCAGCCCGAAGGTATGATCTCAGAGTCACTTGATAACT
TGGAATCCGTGATGCCCAACAAGGTCAGGAAGATTGGCGAGCTCGTGAGA
TACTTGAACACCAATCCTGTGGGTGGCCTTTTGGAGTACGCCCGCTCCCA
TGGCTTTGCTGCTGAATTCAAGTTGGTCGACCAGTCTGGACCTCCTCACG
AGCCCAAGTTCGTTTACCAAGCAAAAGTTGGGGGTCGCTGGTTCCCAGCC
GTCTGCGCACACAGCAAGAAGCAAGGCAAGCAGGAAGCAGCAGATGCGGC
TCTCCGTGTCTTGATTGGGGAGAACGAGAAGGCAGAACGCATGGGTTTCA
CAGAGCTCCCTCTCACTGGCAGCACCTTCCATGACCAGATAGCCATGCTG
AGCCACCGGTGCTTCAACACTCTGACTAACAGCTTCCAGCCTTCCTTGCT
CGGCCGCAAGATTCTGGCCGCCATCGTTATGAAAAAAGACTCTGAGGACA
TGGGTGTCGTCGTCAGCTTGGGAACAGGGAATCGCTGTGTGAAAGGAGAT
TCTCTCAGCCTGAAGGGAGAAACTGTCAATGACTGCCATGCAGAAATCAT
CTCCCGGAGAGGCTTCATCAGGTTTCTCTACAGTGAGTTAATGAAATACA
ACCCCCAGACTGCGAAGGATAGTATATTTGAACCTGCTAAGGGAGGAGAA
AAGCTCCAAATAAAAAAGACTGTGTCATTCCATCTCTATATCAGCACCGC
TCCATGTGGAGATGGTGCCCTCTTTGACAAGTCCTGCAGCGACCGTGCTA
TGGAAAGCACAGACTCCCGCCACTACCCTGTCTTTGAGAATCCCAAACAA
GGCAAGCTCCGCACCAAGGTGGAAAACGGAGAAGGCACAATCCCTGTGGA
ATCCAGTGACATTGTACCTACGTGGGATGGCATTCGGCTCGGGGAGAGAC
TCCGTACCATGTCCTGTAGTGACAAAATCCTACGCTGGAATGTGCTGGGC
CTGCAAGGGGCACTGTTGACCCACTTCCTGCAGCCCATTTATCTCAAATC
TGTTACATTGGGTTACCTTTTCAGCCAAGGGCATCTGACCCGGGCTATTT
GCTGTCGTGTGACAAGAGATGGGAGTGCATTTGAGGATGGACTACGACAT
CCCTTTATTGTCAACCACCCCAAGGTTGGCAGAGTCAGCGTATATGATTC
CAAAAGGCAATCTGGGAAGACTAAGGAGACAAGCGTCAACTGGTGTCTGG
CTGATGGCTATGACCTGGAGATCCTGGATGGTACCAGAGGCACCGTGGAT
GGGCCACGGAATGAATTGTCCCGGGTCTCCAAAAAGAACATTTTTCTTCT
ATTTAAGAAGCTCTGCTCCTTCCGTTACCGCAGGGATCTACTGAGACTCT
CCTATGGTGAGGCCAAGAAAGCTGCCCGTGACTACGAGACGGCCAAGAAC
TACTTCAAAAAAAGCTTGAAGGATATGGGCTATGGGAACTGGATTAGCAA
ACCCCAGGAGGAAAAGAACTTTTATCTCTGCCCAGTATAGTATGCTCCGG
TGACAGATGGATTAGGGTGTGTCATACTAGGGTGAGAGAGAGGTAGGTCG
TAGCGTTCCTCATCACATGGTCAGGGGATTTTTTTTTCCTCCTTTTTTTT
CTTTTTAAGCCATAATTGGTGATACTGAAAACTTTGGGTTCCCATTTATC
CTGCTTTCTTTGGGATTGCTAGGCAAGGTCTGGCCAGGCCCCCCTTTTTT
TCCCCCAAGTGAAGAGGTAGAAACCTAAGAAGTTATCTTTTCTTTCTATA
ACTCAAAGCATACATAGTCACTGAGCACCTGCAGTCCATTTCCTCTTAAA
AGTTTTTTTTTTTTTCCATTTCCTTTCCCTTTGTGTTTGCTACACTGACC
TCTTGTGGTCTTGATTAGGTTTCAGTCAATTCTGGATCATGTCCGAGACT
GATGATTTCTTTTGTGGATTACGCAGACCCCTCTACTTCCCCCTTCCCCT
TCTGAGGTTCTTTCCTTGTGATCAGAATTTCTCCTTTTCTCCCTCAGAGG
GTAAAAAGGTGAACTTAAAGGATCTGGTGAAACATTTGTAAGGGTAGGAG
TTGAAAACTGCAGTTCCCAGTGTCACGGAAGTGTGATTGGAGCCTACAGA
TAATGCCCTGCCATCCTCCCATCCTGCACTTTAGCCAGCTGCAGGGCGGG
CAAGGCAAGGAAAGCTGCTTCCCTGGAAGTGTGCCCCTTTCCCCGGCAGC
AGGGAAGTCTAGAACCAGCCAGACTGGGTTAAGGGAGTTGCCCAAGCCAT
AGCGGAGGTTTCACCCAGCAAGGTGACACAGACCACTTCCCAGGAGCACG
GGCATGCCTTGGGATATTGCCAAGCTTCCAGCTGCCTCTTCTCCTAAAGC
ACTCCTAGGAATTTGCCCTGCAAATGCTGGGCGTGCACCCTAGCCAACGT
AGGAAAAAATCCAGGACAAATCCTAGAGGGTAGAAAATCATCTCTGCTCA
GATAATCATGACTTAGCAAGAATAAGGGCAAAAAATCCTGTTGGCTCACG
TCACTGTTCTACCCTGCATAATATCTCTCATGACAGTGACACCAAGAGAA
GTTGACTAAGTCATATGTAAATTAGGAGTGTTTAAAAGAATGCCATAGAT
GTTGATTCTTAACTGCTACAGACAACCTATAATTGAGCAGATTTAAAATT
CAGGCATACTTTTCCATTTATCCAAGTGCTTTCATTTTTCCAGATGGCTT
CAGAAGTAGGCTCGTGGGCAGAGCGCAGACCTGATCTTTATAGTGTTGAC
ATAGAAAGCAGTAGTAGTGGGTGAAAGGACAGGTTGTCTTCAAACTCTGT
GAGGTGGAATCTTTTGTCTACACCTCCATGAACATTGACTCGTGTGTTCA
GAGCCTTTGGCCTCCCTGTGGAGTCTGGCTCTCTGGCTCCTGTGCATTCT
TTGAATAGTCACTCGTAAAAATTGTCAATGCTTGAAACTATTTCTTTTAC
TCATGTTGAAGGGACTTTGTTGACTTTTAGAGTGTTGGTCATGACTCCAA
GAGCAGAGCAGGGAAGAGCCCAAGCATAGACTTGAGTGCCGTGATGGCTG
CGGTCCAGTTTTGTGATTCTGCTTTTACGTGTTACTTGGTAACAGTCAGC
TAGACACACTCAGGAGGACTACTGAGGCTCTGCGACCTTCAGGACCTGAG
CCTGCCTCTCTCCTTTAGATGACAGACCTTCATCTGAGAATGTGCTGAGC
CAGCACCCTCAGATGATTTCCCTCCAAACTGCTGACTAGGTCACCCTCTG
TCTGGTAGAGACATTGACATCTTTGCTTTTATTCTATACCCTCGGTACTT
TTGACCAAAAAATGACCAAAATAAGAAAATGCAACTTTAAAAAATAGACT
AAGGATGCCTTTGCAGAACACCAAAGCATCCCAAGGAACCGGTAGGGAAA
TGATGCCTGTCTCCTGGGGTAGAAGAGGCCTGCTCCCTGGCTCTGGGTCT
GCTGGGGGCACAGTAAATCAGTCTTGGCACCCACATCCAGGTCAGAGAGG
TCTGTGGTTCTCAGCATCAGAAGGCAGCGCAGCCCCTCTCCAGGCTACAG
GGTTGTCACCTGCTGAGTCCTCAAGCTGTTTGGCCTCTCTGGTCCGTCTT
GGGCATTAGATTCTCCAGCAGAGCTCTGGCCAGCTGCCTCCTCTTTAACT
GGGAACACGGGCTCTCACAAGATCAGAACCCCCACTCACCCCTGAGATCT
TATCTAGCAAGCCTGTAGTATTCCGTTTCTGTTGTAGGAAGAGCGCGAGG
CATCCCTGAATTCCATGCATCTGCTGGAAATGAGCCGTGTCAGATCGCAC
ACCCCCGCGCCCCCATGCCCCTCTGAGTCACACGGGACAGAGGAGGCAGA
GCTTCCGCCCACTGTTATCTTCAATTTCTTTGTCCAGTCTTTTGTTTTTA
ATAAGCAGTGCCCCTCCCCACTCTTCTTTTTAATGATTTTTGTAGTTGAT
TTGTCTGAACTGTGGCTACTGTGCATTCCTTGAATAATCACTTGTAAAAA
TTGTCAGTGCTTGAAGCTGTTTCCTTTACTCACATTGAAGGGACTTTGTT
GGTTTTTTGGAGTCTTGGTTTTGACTCCAAGAACAGAGTCAGGAAGACCC
CAAGCATAGACTTGGGTACCGTGAAAATGGCTGCAGTCCAGTTTTATGAT
TCCGCTTTTATGTGTCCCTTGATAACAGTGACTTAACAATATACATTCCT
CATAAATAAAAAAAAAACCAAGAATCTGAATTCTTA

SEQ ID NO: 54 - NHP ADAR1 - Transcript Variant X5 Coding Sequence

ATGAACCCGCGGCAGGGGTATTCCCTCAACGGATACTACCCCCATCCATT
TCAAGGCTATGAGCACAGAAAGCTCAGATACCAGCAGCCTGGGCCAGGAT
CTTCCCCCAATAGTTTCCTGCTTAAGCAAATAGAGTTTCTCAAGGGGCAG
ATCCCAGAAGCGCCGGTGATTGGAAAGCAGACACCATCACTGCCACCTTC
CCTCCCAGGACTCCAGCCAAGGTTTCCAGTACTACCTGCCTCTAGTACCA
GAGGCAGGCAAGTGGATATCAGGGGTGTCCCCAGGTGCGTGCATCTCGGA
AGTCAGGGGCTCCAGAGAGGGTTCCAGCATCCTTCACCACGTGGCAGGAT
TCCGCCACGGAGAGGTGTTGATTGCCTTTCCTCACAATTCCAGGAACTGA
GTATCAACCAAGATCAGGAACAAAGGATCTTAAAGTTCTTGGAAGAGCTT
GGGGAAGGGAAGGCCACTACAGCACATGATCTGTCTTGGAAACTGGGGGC
TCCAAAGAAAGAAATCAATCGAGTTTTATACTCCCTGGCAAAGAAGGGCA
AGCTACAGAAAGAGGCAGGAACACCCCCTTTGTGGAAAATCGCGGTCTCC
ACTCAGGCTTGGAACCAGCACAGCAGAGTGGTAAGACCAGACGGTCATAG
CCAAGGAGCCCCAAACTCAGACCCAAATTTGGAACCTGAAGACAGAAACT
CCACATCTGTCTCAGAAGATCTTGAGCCTTTTATCTCAGTCTCAGCTCAG
GCTTGGAACCAGCACAGCGGAGTGGTAAGACCAGACGGTCATAGCCAAGG
AGCCCCAAACTCAGAGTCGAGTTTGGAACCTGAAGACAGAAACTCCACAT
CTGCCTTGGAAGATCCTCTTGAGTTTTTTGACATGGCTGAGATCAAGGAG
AAAATCTGCGACTATCTCTTCAATGTGTCTGACTCCTCTGCCCTGAATTT
GGCTAAAAATATTGGCCTCACCAAGGCCCGAGATATAAATGCCGTGCTAA
TTGACATGGAAAGGCAGGGGGATGTCTATAGACAAGGGACAACCCCTCCC
ATATGGCATTTGACAGACAAGAAGCGAGAGAGGATGCAAATCAAGAGAAA
TACGAACAGTGTTCCTGAAACCGCTCCAGCTGCAATCCCTGAGACCAAAA
GAAACACAGAGTTCCTCACCTGTAATATACCCACATCAAATGCCTCAAAT
AACATAGTAACCACCGAAAAAGTGGAGAATGGGCAGGAACCTGTCATAAA
GTTAGAAAACAGTCAGGAGGCAAGACCAGAACCAGTAAGACTGAAACCAC
CTGTTCATAACAATGGCCCCTCAAAAGCAGGGTATGTTGACTTTGAAAAT
GGCCAGTGGGCCACAGATGACATCCCAGATGACTTGAATAGTATCCGCGC
AGCACCAGGTGAGTTTCGAGCCATCATGGAGATGCCCTCCTTCTACAGTC
ATGGCTTGCCACGGTGTTCACCCTACAAGAAACTGACAGAGTGCCAGCTG
AAGAACCCCATCAGCGGGCTGTTAGAATATGCCCAGTTCGCTAGTCAAAC
CTGTGAGTTCAACATGATAGAGCAGAGTGGACCACCCCATGAACCTCGAT
TTAAATTCCAGGTTGTCATCAATGGCCGAGAGTTTCCCCCAGCTGAAGCT
GGAAGCAAGAAAGTGGCCAAGCAGGATGCAGCTATGAAAGCCATGACAAT
TCTGCTAGAGGAAGCCAAAGCCAAGGACAGTGGAAAATCAGAAGAATCAT
CCCACTATTCCACAGAAAAAGAATCAGAGAAGACCACAGAGTCCCAGACC
CCCACCCCTTCAGCTACATCCTTCTTTTCTGGGAAGAGCCCCGTCACCAC
ACTGCTCGAGTGTATGCACAAATTGGGGAACTCCTGCGAATTCCGTCTCC
TGTCCAAAGAAGGCCCTGCCCATGAGCCCAAGTTCCAGTACTGTGTTGCA
GTGGGAGCCCAAACTTTCCCCAGTGTGAGTGCCCCCAGCAAGAAAGTGGC
AAAGCAGATGGCCGCAGAGGAAGCCATGAAGGCCCTGCATGGGGAGGCAA
CCAACTCCATGACTTCTGATGACCAGCCCGAAGGTATGATCTCAGAGTCA
CTTGATAACTTGGAATCCGTGATGCCCAACAAGGTCAGGAAGATTGGCGA
GCTCGTGAGATACTTGAACACCAATCCTGTGGGTGGCCTTTTGGAGTACG
CCCGCTCCCATGGCTTTGCTGCTGAATTCAAGTTGGTCGACCAGTCTGGA
CCTCCTCACGAGCCCAAGTTCGTTTACCAAGCAAAAGTTGGGGGTCGCTG
GTTCCCAGCCGTCTGCGCACACAGCAAGAAGCAAGGCAAGCAGGAAGCAG
CAGATGCGGCTCTCCGTGTCTTGATTGGGGAGAACGAGAAGGCAGAACGC
ATGGGTTTCACAGAGCTCCCTCTCACTGGCAGCACCTTCCATGACCAGAT
AGCCATGCTGAGCCACCGGTGCTTCAACACTCTGACTAACAGCTTCCAGC
CTTCCTTGCTCGGCCGCAAGATTCTGGCCGCCATCGTTATGAAAAAAGAC
TCTGAGGACATGGGTGTCGTCGTCAGCTTGGGAACAGGGAATCGCTGTGT
GAAAGGAGATTCTCTCAGCCTGAAGGGAGAAACTGTCAATGACTGCCATG
CAGAAATCATCTCCCGGAGAGGCTTCATCAGGTTTCTCTACAGTGAGTTA
ATGAAATACAACCCCCAGACTGCGAAGGATAGTATATTTGAACCTGCTAA
GGGAGGAGAAAAGCTCCAAATAAAAAAGACTGTGTCATTCCATCTCTATA
TCAGCACCGCTCCATGTGGAGATGGTGCCCTCTTTGACAAGTCCTGCAGC
GACCGTGCTATGGAAAGCACAGACTCCCGCCACTACCCTGTCTTTGAGAA
TCCCAAACAAGGCAAGCTCCGCACCAAGGTGGAAAACGGAGAAGGCACAA
TCCCTGTGGAATCCAGTGACATTGTACCTACGTGGGATGGCATTCGGCTC
GGGGAGAGACTCCGTACCATGTCCTGTAGTGACAAAATCCTACGCTGGAA
TGTGCTGGGCCTGCAAGGGGCACTGTTGACCCACTTCCTGCAGCCCATTT
ATCTCAAATCTGTTACATTGGGTTACCTTTTCAGCCAAGGGCATCTGACC
CGGGCTATTTGCTGTCGTGTGACAAGAGATGGGAGTGCATTTGAGGATGG
ACTACGACATCCCTTTATTGTCAACCACCCCAAGGTTGGCAGAGTCAGCG
TATATGATTCCAAAAGGCAATCTGGGAAGACTAAGGAGACAAGCGTCAAC
TGGTGTCTGGCTGATGGCTATGACCTGGAGATCCTGGATGGTACCAGAGG
CACCGTGGATGGGCCACGGAATGAATTGTCCCGGGTCTCCAAAAAGAACA
TTTTTCTTCTATTTAAGAAGCTCTGCTCCTTCCGTTACCGCAGGGATCTA
CTGAGACTCTCCTATGGTGAGGCCAAGAAAGCTGCCCGTGACTACGAGAC
GGCCAAGAACTACTTCAAAAAAAGCTTGAAGGATATGGGCTATGGGAACT
GGATTAGCAAACCCCAGGAGGAAAAGAACTTTTATCTCTGCCCAGTATAG

SEQ ID NO: 55 - NHP ADAR1 - Transcript Variant X5 Amino Acid Sequence

MNPRQGYSLNGYYPHPFQGYEHRKLRYQQPGPGSSPNSFLLKQIEFLKGQ
IPEAPVIGKQTPSLPPSLPGLQPRFPVLPASSTRGRQVDIRGVPRCVHLG
SQGLQRGFQHPSPRGRIPPRRGVDCLSSQFQELSINQDQEQRILKFLEEL
GEGKATTAHDLSWKLGAPKKEINRVLYSLAKKGKLQKEAGTPPLWKIAVS
TQAWNQHSRVVRPDGHSQGAPNSDPNLEPEDRNSTSVSEDLEPFISVSAQ
AWNQHSGVVRPDGHSQGAPNSESSLEPEDRNSTSALEDPLEFFDMAEIKE
KICDYLFNVSDSSALNLAKNIGLTKARDINAVLIDMERQGDVYRQGTTPP
IWHLTDKKRERMQIKRNTNSVPETAPAAIPETKRNTEFLTCNIPTSNASN
NIVTTEKVENGQEPVIKLENSQEARPEPVRLKPPVHNNGPSKAGYVDFEN
GQWATDDIPDDLNSIRAAPGEFRAIMEMPSFYSHGLPRCSPYKKLTECQL
KNPISGLLEYAQFASQTCEFNMIEQSGPPHEPRFKFQVVINGREFPPAEA
GSKKVAKQDAAMKAMTILLEEAKAKDSGKSEESSHYSTEKESEKTTESQT
PTPSATSFFSGKSPVTTLLECMHKLGNSCEFRLLSKEGPAHEPKFQYCVA
VGAQTFPSVSAPSKKVAKQMAAEEAMKALHGEATNSMTSDDQPEGMISES
LDNLESVMPNKVRKIGELVRYLNTNPVGGLLEYARSHGFAAEFKLVDQSG
PPHEPKFVYQAKVGGRWFPAVCAHSKKQGKQEAADAALRVLIGENEKAER
MGFTELPLTGSTFHDQIAMLSHRCFNTLTNSFQPSLLGRKILAAIVMKKD
SEDMGVVVSLGTGNRCVKGDSLSLKGETVNDCHAEIISRRGFIRFLYSEL
MKYNPQTAKDSIFEPAKGGEKLQIKKTVSFHLYISTAPCGDGALFDKSCS
DRAMESTDSRHYPVFENPKQGKLRTKVENGEGTIPVESSDIVPTWDGIRL
GERLRTMSCSDKILRWNVLGLQGALLTHFLQPIYLKSVTLGYLFSQGHLT
RAICCRVTRDGSAFEDGLRHPFIVNHPKVGRVSVYDSKRQSGKTKETSVN
WCLADGYDLEILDGTRGTVDGPRNELSRVSKKNIFLLFKKLCSFRYRRDL
LRLSYGEAKKAARDYETAKNYFKKSLKDMGYGNWISKPQEEKNFYLCPV

In some embodiments, provided non-human animals (e.g., rodents, e.g., rats or mice) comprise a polynucleotide or an exogenously derived ADAR1 locus from any of the sequences disclosed herein. In some embodiments, provided non-human animals (e.g., rodents, e.g., rats or mice) comprise an exogenously derived ADAR1 locus created through recombination of any portion of the sequences disclosed herein.

### Exemplary Promoters

In some embodiments, a construct (e.g., a construct harboring human ADAR1 gene) comprises a promoter. The term “promoter” refers to a DNA sequence recognized by enzymes/proteins that can promote and/or initiate transcription of an operably linked gene (e.g., a human ADAR1 gene). For example, a promoter typically refers to, e.g., a nucleotide sequence to which an RNA polymerase and/or any associated factor binds and from which it can initiate transcription. Thus, in some embodiments, a construct (e.g., a targeting construct and/or vector comprising a human ADAR1 gene) comprises a promoter operably linked to one of the non-limiting example promoters described herein.

In some embodiments, a promoter is an inducible promoter, a constitutive promoter, a mammalian cell promoter, a viral promoter, a chimeric promoter, an engineered promoter, a tissue-specific promoter, an insertional site endogenous promoter, or any other type of promoter known in the art. In some embodiments, a promoter is a RNA polymerase II promoter, such as a mammalian RNA polymerase II promoter. In some embodiments, a promoter is a RNA polymerase III promoter, including, but not limited to, a HI promoter, a human U6 promoter, a mouse U6 promoter, or a swine U6 promoter. A promoter will generally be one that is able to promote transcription in an inner ear cell. In some embodiments, a promoter is a cochlea-specific promoter or a cochlea-oriented promoter. In some embodiments, a promoter is a hair cell specific promoter, or a supporting cell specific promoter.

A variety of promoters are known in the art, which can be used herein. Non-limiting examples of promoters that can be used herein include: human EFlα, human cytomegalovirus (CMV) (U.S. Pat. No. 5,168,062), human ubiquitin C (UBC), mouse phosphoglycerate kinase 1, polyoma adenovirus, simian virus 40 (SV40), β-globin, β-actin, α-fetoprotein, γ-globin, β-interferon, γ-glutamyl transferase, mouse mammary tumor virus (MMTV), Rous sarcoma virus, rat insulin, glyceraldehyde-3-phosphate dehydrogenase, metallothionein II (MT II), amylase, cathepsin, MI muscarinic receptor, retroviral LTR (e.g., human T-cell leukemia virus HTLV), AAV ITR, interleukin-2, collagenase, platelet-derived growth factor, adenovirus 5 E2, stromelysin, murine MX gene, glucose regulated proteins (GRP78 and GRP94), α-2-macroglobulin, vimentin, MHC class I gene H-2_{K} b, HSP70, proliferin, tumor necrosis factor, thyroid stimulating hormone a gene, immunoglobulin light chain, T-cell receptor, HLA DQa and DQ, interleukin-2 receptor, MHC class II, MHC class II HLA-DRa, muscle creatine kinase, prealbumin (transthyretin), elastase I, albumin gene, c-fos, c-HA-ras, neural cell adhesion molecule (NCAM), H2B (TH2B) histone, rat growth hormone, human serum amyloid (SAA), troponin I (TN I), duchenne muscular dystrophy, human immunodeficiency virus, and Gibbon Ape Leukemia Virus (GALV) promoters. Additional examples of promoters are known in the art. See, e.g., Lodish, Molecular Cell Biology, Freeman and Company, New York 2007.

In some embodiments, a promoter is the CMV immediate early promoter. In some embodiments, the promoter is a CAG promoter or a CAG/CBA promoter. In certain embodiments, a promoter comprises a CMV/CBA enhancer/promoter construct. In certain embodiments, a promoter comprises a CAG promoter or CMV/CBA/SV-40 enhancer/promoter construct.

The term “constitutive” promoter refers to a nucleotide sequence that, when operably linked with a nucleic acid encoding a protein (e.g., a pendrin protein), causes RNA to be transcribed from the nucleic acid in a cell under most or all physiological conditions.

Examples of constitutive promoters include, without limitation, the retroviral Rous sarcoma virus (RSV) LTR promoter, the cytomegalovirus (CMV) promoter (see, e.g., Boshart et al, Cell 41:521-530, 1985), the SV40 promoter, the dihydrofolate reductase promoter, the beta-actin promoter, the phosphoglycerol kinase (PGK) promoter, and the EFl-alpha promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Additional examples of inducible promoters are known in the art.

Examples of inducible promoters regulated by exogenously supplied compounds include the zinc-inducible sheep metallothionein (MT) promoter, the dexamethasone (Dex)-inducible mouse mammary tumor virus (MMTV) promoter, the T7 polymerase promoter system (WO 98/10088); the ecdysone insect promoter (No et al, Proc. Natl. Acad Sci. US.A. 93:3346-3351, 1996), the tetracycline-repressible system (Gossen et al, Proc. Natl. Acad Sci. US.A. 89:5547-5551, 1992), the tetracycline-inducible system (Gossen et al, Science 268:1766-1769, 1995, see also Harvey et al, Curr. Opin. Chem. Biol. 2:512-518, 1998), the RU486-inducible system (Wang et al, Nat. Biotech. 15:239- 243, 1997, and Wang et al, Gene Ther. 4:432-441, 1997), and the rapamycin-inducible system (Magari et al. J Clin. Invest. 100:2865-2872, 1997).

The term “tissue-specific” promoter refers to a promoter that is active only in certain specific cell types and/or tissues (e.g., transcription of a specific gene occurs only within cells expressing transcription regulatory and/or control proteins that bind to the tissue-specific promoter).

In some embodiments, regulatory and/or control sequences impart tissue-specific gene expression capabilities. In some cases, tissue-specific regulatory and/or control sequences bind tissue-specific transcription factors that induce transcription in a tissue-specific manner.

In some embodiments, a tissue-specific promoter is a central nervous system (CNS) specific promoter. Non-limiting examples of CNS specific promoters include but are not limited to promoters or functional portions thereof for genes: Aldh1l1, CaMIIα, Dlx1, Dlx⅚, Gad2, GFAP, Grik4, Lepr, Nes, nNOS, Pdgfrα, PLP1, Pv (Pvalb), Slcl7a6, Sst, Vip, Pcp2, Slc6a3 (DAT), ePet (Fev), Npy2r, Cdh3, and/or Htr6; see e.g., Kim et al., “Mouse Cre-LoxP system: general principles to determine tissue-specific roles of target genes” Laboratory Animal Research (2018) 34(4), 147-159.. In certain embodiments, a CNS specific promoter comprises, or consists of, a nucleotide sequence that is the same as, or has at least has at least 70%, 75%, 80%, 85%, 90%, 92%, 94%, 96%, 98%, or 99% homology with promoters for genes: Aldh1l1, CaMIIα, Dlx1, Dlx⅚, Gad2, GFAP, Grik4, Lepr, Nes, nNOS, Pdgfrα, PLP1, Pv (Pvalb), Slcl7a6, Sst, Vip, Pcp2, Slc6a3 (DAT), ePet (Fev), Npy2r, Cdh3, and/or Htr6.

In some embodiments, a tissue-specific promoter is an ocular cell specific promoter. Non-limiting examples of ocular cell specific promoters include but are not limited to promoters or functional portions thereof for genes: EFS, GRK1, CRX, NRL, and/or RCVRN. In certain embodiments, an ocular system specific promoter comprises, or consists of, a nucleotide sequence that is the same as, or has at least has at least 70%, 75%, 80%, 85%, 90%, 92%, 94%, 96%, 98%, or 99% homology with promoters for genes: EFS, GRK1, CRX, NRL, and/or RCVRN.

In some embodiments, a tissue-specific promoter is a hepatic system specific promoter. Non-limiting examples of hepatic system specific promoters include but are not limited promoters or functional portions thereof for genes: EFS, EF-la, MSCV, PGK, CAG, ALB, and/or SERPINA1. In certain embodiments, a hepatic system specific promoter comprises, or consists of, a nucleotide sequence that is the same as, or has at least has at least 70%, 75%, 80%, 85%, 90%, 92%, 94%, 96%, 98%, or 99% homology with promoters for genes: EFS, EF-la, MSCV, PGK, CAG, ALB, and/or SERPINA1.

In some embodiments, provided nucleic acid constructs comprise a promoter sequence selected from a CAG, a CBA, a CMV, or a CB7 promoter. In certain embodiments, a promoter comprises, or consists of, a nucleotide sequence that is the same as, or has at least has at least 70%, 75%, 80%, 85%, 90%, 92%, 94%, 96%, 98%, or 99% homology with a CAG, a CBA, a CMV, or a CB7 promoter.

In some embodiments of any of the nucleic acid constructs described herein, the first or sole nucleic acid constructs further includes at least one promoter sequence or functional portion thereof selected from CNS, Ocular, and/or Hepatic cell specific promoters.

### Exemplary Enhancers

In some instances, a construct can include an enhancer sequence. In some embodiments,, the term “enhancer” refers to a nucleotide sequence that can increase the level of transcription of a nucleic acid encoding a protein of interest (e.g., a human and/or NHP ADAR1 protein). Enhancer sequences (generally 50-1500 bp in length) generally increase the level of transcription by providing additional binding sites for transcription-associated proteins (e.g., transcription factors). In some embodiments, an enhancer sequence is found within an intronic sequence. Unlike promoter sequences, enhancer sequences can act at much larger distance away from the transcription start site (e.g., as compared to a promoter). Non-limiting examples of enhancers include a RSV enhancer, a CMV enhancer, and/or a SV40 enhancer. In some embodiments, a construct comprises a CMV enhancer, In some embodiments, an SV-40 derived enhancer is the SV-40 T intron sequence. In some embodiments, an enhancer sequence is woodchuck hepatitis virus post-transcriptional regulatory element (WPRE). In some embodiments, an enhancer sequence comprises, or consists of, a nucleotide sequence that is the same as, or has at least has at least 70%, 75%, 80%, 85%, 90%, 92%, 94%, 96%, 98%, or 99% homology with a WPRE nucleic acid sequence as represented by SEQ ID NO: 56.

SEQ ID NO: 56 - Exemplary WPRE nucleic acid sequence

AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTGCTGTCTCTTTATGAGGAGTTGTGGCCCGTTGTCAGGCAACG
TGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAGG
GGCTCGGCTGTTGGGCACTGACAATTCCGTGGTGTTGTCGGGGAAATCAT
CGTCCTTTCCTTGGCTGCTCGCCTGTGTTGCCACCTGGATTCTGCGCGGG
ACGTCCTTCTGCTACGTCCCTTCGGCCCTCAATCCAGCGGACCTTCCTTC
CCGCGGCCTGCTGCCGGCTCTGCGGCCTCTTCCGCGTCTTCGCCTTCGCC
CTCAGACGAGTCGGATCTCCCTTTGGGCCGCCTCCCCGC

### Exemplary Coding Sequence Flanking Untranslated Regions

In some embodiments, any of the constructs described herein can include an untranslated region (UTR), such as a 5′ UTR or a 3′ UTR. UTRs of a gene are transcribed but not translated. A 5′ UTR starts at the transcription start site and continues to the start codon but does not include the start codon. A 3′ UTR starts immediately following the stop codon and continues until the transcriptional termination signal. The regulatory and/or control features of a UTR can be incorporated into any of the constructs, compositions, kits, or methods as described herein to enhance or otherwise modulate the expression of an ADAR1 protein.

Natural 5′ UTRs include a sequence that plays a role in translation initiation. in some embodiments, a 5′ UTR can comprise sequences, like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), and the start codon is followed by another “G”. In certain embodiments, a KOZAK sequence is GCCACC. The 5′ UTRs have also been known to form secondary structures that are involved in elongation factor binding.

In some embodiments, a 5′ UTR is included in any of the constructs described herein. Non-limiting examples of 5′ UTRs, including those from the following genes: albumin, serum amyloid A, Apolipoprotein A/B/E, transferrin, alpha fetoprotein, erythropoietin, Factor VIII, and ADAR1 can be used to enhance expression of a nucleic acid molecule, such as an mRNA.

In some embodiments, a 5′ UTR from an mRNA that is transcribed by a cell in the CNS can be included in any of the constructs, compositions, kits, and methods described herein. In some embodiments, a 5′ UTR is derived from the endogenous ADAR1 gene loci and may include all or part of the endogenous sequence exemplified by SEQ ID NO: 1. In some embodiments, a 5′ UTR sequence is at least 85%, 90%, 95%, 98% or 99% identical to a 5′ UTR sequence for any one of SEQ ID NOs: 2, 7, 10, 13, 17, 18, 21, 22, 23, or 24.

3′ UTRs are found immediately 3′ to the stop codon of the gene of interest. In some embodiments, a 3′ UTR from an mRNA that is transcribed by a cell in the CNS can be included in any of the constructs, compositions, kits, and methods described herein. In some embodiments, a 3′ UTR is derived from the endogenous ADAR1 gene loci and may include all or part of the endogenous sequence exemplified by SEQ ID NO: 1. In some embodiments, a 3′ UTR sequence is at least 85%, 90%, 95%, 98% or 99% identical to a 5′ UTR sequence for any one of SEQ ID NOs: 2, 7, 10, 13, 17, 18, 21, 22, 23, or 24.

3′ UTRs are known to have stretches of adenosines and uridines (in the RNA form) or thymidines (in the DNA form) embedded in them. These AU-rich signatures are particularly prevalent in genes with high rates of turnover. Based on their sequence features and functional properties, the AU-rich elements (AREs) can be separated into three classes (Chen et al., Mal. Cell. Biol. 15:5777-5788, 1995; Chen et al., Mal. Cell Biol. 15:2010-2018, 1995): Class I AREs contain several dispersed copies of an AUUUA motif within U-rich regions. For example, c-Myc and MyoD mRNAs contain class I AREs. Class II AREs possess two or more overlapping UUAUUUA(U/A) (U/A) nonamers. GM-CSF and TNF-alpha mRNAs are examples that contain class II AREs. Class III AREs are less well defined. These U-rich regions do not contain an AUUUA motif, two well-studied examples of this class are c-Jun and myogenin mRNAs.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

In some embodiments, the introduction, removal, or modification of 3′ UTR AREs can be used to modulate the stability of an mRNA encoding an ADAR1 protein. In other embodiments, AREs can be removed or mutated to increase the intracellular stability and thus increase translation and production of an ADAR1 protein. In other embodiments, non-ARE sequences may be incorporated into the 5′ or 3′ UTRs. In some embodiments, introns or portions of intron sequences may be incorporated into the flanking regions of the polynucleotides in any of the constructs, compositions, kits, and methods provided herein. Incorporation of intronic sequences may increase protein production as well as mRNA levels.

### Exemplary Internal Ribosome Entry Sites (IRES)

In some embodiments, a construct encoding an ADAR1 protein can include an internal ribosome entry site (IRES). An IRES forms a complex secondary structure that allows translation initiation to occur from any position with an mRNA immediately downstream from where the IRES is located (see, e.g., Pelletier and Sonenberg, Mal. Cell. Biol. 8(3):1103-1112, 1988).

There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HAV), hepatitis C virus (HCV), and poliovirus (PV). See e.g., Alberts, Molecular Biology of the Cell, Garland Science, 2002; and Hellen et al., Genes Dev. 15(13):1593-612, 2001.

In some embodiments, the IRES sequence that is incorporated into a construct that encodes an ADAR1 protein is the foot and mouth disease virus (FMDV) 2A sequence. The Foot and Mouth Disease Virus 2A sequence is a small peptide (approximately 18 amino acids in length) that has been shown to mediate the cleavage of polyproteins (Ryan, MD et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999). The cleavage activity of the 2A sequence has previously been demonstrated in artificial systems including plasmids and gene therapy constructs (AAV and retroviruses) (Ryan et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999; de Felipe et al., Gene Therapy 6:198-208, 1999; de Felipe et al., Human Gene Therapy I I: 1921-1931, 2000; and Klump et al., Gene Therapy 8:811-817, 2001).

An IRES can be utilized in an any constructs described herein. In some embodiments, an IRES can be part of a composition comprising more than one construct. In some embodiments, an IRES is used to produce more than one polypeptide from a single gene transcript.

### Exemplary Splice Sites

In some embodiments, any of the constructs provided herein can include splice donor and/or splice acceptor sequences, which are functional during RNA processing occurring during transcription. In some embodiments, splice sites are involved in trans-splicing. In some embodiments, a construct provided herein can include a splice acceptor sequence that is at least 85%, 90%, 95%, 98% or 99% identical to a splice acceptor sequence as represented by SEQ ID NO: 57.

SEQ ID NO: 57 - Exemplary splice acceptor nucleic acid sequence

gtgacctgcacgtctagggcgcagtagtccagggtttccttgatgatgtc
atacttatcctgtcccttttttttccacagctcgcggttgaggacaaact
cttcgcggtctttccagt

### Exemplary Polyadenylation Sequences

In some embodiments, a construct provided herein can include a polyadenylation (poly(A)) signal sequence. Most nascent eukaryotic mRNAs possess a poly(A) tail at their 3′ end, which is added during a complex process that includes cleavage of the primary transcript and a coupled polyadenylation reaction driven by the poly(A) signal sequence (see, e.g., Proudfoot et al., Cell 108:501-512, 2002). A poly(A) tail confers mRNA stability and transferability (Molecular Biology of the Cell, Third Edition by B. Alberts et al., Garland Publishing, 1994). In some embodiments, a poly(A) signal sequence is positioned 3′ to the coding sequence.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. A 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In some embodiments, a poly(A) tail is added onto transcripts that contain a specific sequence, e.g., a poly(A) signal. A poly(A) tail and associated proteins aid in protecting mRNA from degradation by exonucleases. Polyadenylation also plays a role in transcription termination, export of the mRNA from the nucleus, and translation. Polyadenylation typically occurs in the nucleus immediately after transcription of DNA into RNA, but also can occur later in the cytoplasm. After transcription has been terminated, an mRNA chain is cleaved through the action of an endonuclease complex associated with RNA polymerase. A cleavage site is usually characterized by the presence of the base sequence AAUAAA near the cleavage site. After the mRNA has been cleaved, adenosine residues are added to the free 3′ end at the cleavage site.

As used herein, a “poly(A) signal sequence” or “polyadenylation signal sequence” is a sequence that triggers the endonuclease cleavage of an mRNA and the addition of a series of adenosines to the 3′ end of the cleaved mRNA.

There are several poly(A) signal sequences that can be used, including those derived from bovine growth hormone (bGH) (Woychik et al., Proc. Natl. Acad Sci. US.A. 81(13):3944-3948, 1984; U.S. Pat. No. 5,122,458), mouse-β-globin, mouse-α-globin (Orkin et al., EMBO J 4(2):453-456, 1985; Thein et al., Blood 71(2):313-319, 1988), human collagen, polyoma virus (Batt et al., Mal. Cell Biol. 15(9):4783-4790, 1995), the Herpes simplex virus thymidine kinase gene (HSV TK), IgG heavy-chain gene polyadenylation signal (US 2006/0040354,), human growth hormone (hGH) (Szymanski et al., Mal. Therapy 15(7):1340-1347, 2007), the group consisting of SV40 poly(A) site, such as the SV40 late and early poly(A) site (Schek et al., Mal. Cell Biol. 12(12):5386-5393, 1992).

The poly(A) signal sequence can be AATAAA. The AATAAA sequence may be substituted with other hexanucleotide sequences with homology to AATAAA and that are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414).

In some embodiments, a poly(A) signal sequence can be a synthetic polyadenylation site (see, e.g., the pCl-neo expression construct of Promega that is based on Levitt el al, Genes Dev. 3(7):1019-1025, 1989). In some embodiments, a poly(A) signal sequence is the polyadenylation signal of soluble neuropilin-1 (sNRP) (AAATAAAATACGAAATG) (see, e.g., WO 05/073384). In some embodiments, a poly(A) signal sequence comprises or consists of bGHpA. In some embodiments, a poly(A) signal comprises or consists of SEQ ID NO: 58 or SEQ ID NO: 59. In some embodiments, a poly(A) signal sequence comprises or consists of the SV40 poly(A) site. In some embodiments, a poly(A) signal comprises or consists of SEQ ID NO: 60. Additional examples of poly(A) signal sequences are known in the art. In some embodiments, a poly(A) sequence is at least 85%, 90%, 95%, 98% or 99% identical to the poly(A) sequence represented by any one of SEQ ID NOs: 58-60.

SEQ ID NO: 58 - Exemplary bGH poly(A) signal sequence

CTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGA
GGAAATTGCATCGCATTGTCTGAGTAGGTGTCATTCTATTCTGGGGGGTG
GGGTGGGGCAGGACAGCAAGGGGGAGGATTGGGAAGACAATAGCAGGCAT
GCTGGGGATGCGGTGGGCTCTATGG

SEQ ID NO: 59 - Exemplary bGH poly(A) signal sequence

CTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGA
GGAAATTGCATCGCATTGTCTGAGTAGGTGTCATTCTATTCTGGGGGGTG
GGGTGGGGCAGGACAGCAAGGGGGAGGATTGGGAAGACAATGGCAGGCAT
GCTGGGGA

SEQ ID NO: 60 - Exemplary SV40 poly(A) signal sequence

AACTTGTTTATTGCAGCTTATAATGGTTACAAATAAAGCAATAGCATCAC
AAATTTCACAAATAAAGCATTTTTTTCACTGCATTCTAGTTGTGGTTTGT
CCAAACTCATCAATGTATCTTA

### Exemplary Destabilization Domains

In some embodiments, any of the constructs provided herein can optionally include a sequence encoding a destabilizing domain (“a destabilizing sequence”) for temporal control of protein expression. Non-limiting examples of destabilizing sequences include sequences encoding a FK506 sequence, a dihydrofolate reductase (DHFR) sequence, or other exemplary destabilizing sequences.

In the absence of a stabilizing ligand, a protein sequence operatively linked to a destabilizing sequence is degraded by ubiquitination. In contrast, in the presence of a stabilizing ligand, protein degradation is inhibited, thereby allowing the protein sequence operatively linked to the destabilizing sequence to be actively expressed. As a positive control for stabilization of protein expression, protein expression can be detected by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

Additional examples of destabilizing sequences are known in the art. In some embodiments, the destabilizing sequence is a FK506- and rapamycin-binding protein (FK12) sequence, and the stabilizing ligand is Shield-1 (Shld1) (Banaszynski et al. (2012) Cell 126(5): 995-1004). In some embodiments, a destabilizing sequence is a DHFR sequence, and a stabilizing ligand is trimethoprim (TMP) (Iwamoto et al. (2010) Chem Biol 17:981-988).

In some embodiments, a destabilizing sequence is a FK12 sequence, and a presence of a nucleic acid construct carrying the FK12 gene in a subject cell (e.g., a rodent cell, e.g., a rat or mouse cell) is detected by western blotting. In some embodiments, a destabilizing sequence can be used to verify the temporally-specific activity of any of the nucleic acid constructs described herein.

### Exemplary Reporter Sequences or Elements

In some embodiments, constructs provided herein can optionally include a sequence encoding a reporter polypeptide and/or protein (“a reporter sequence”). Non-limiting examples of reporter sequences include DNA sequences encoding: a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences are known in the art. When associated with control elements which drive their expression, the reporter sequence can provide signals detectable by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

In some embodiments, a reporter sequence is the LacZ gene, and the presence of a construct carrying the LacZ gene in a non-human cell (e.g., a rodent cell, e.g., a rat or mouse cell) is detected by assays for beta-galactosidase activity. When the reporter is a fluorescent protein (e.g., green fluorescent protein) or luciferase, the presence of a construct carrying the fluorescent protein or luciferase in a non-human cell (e.g., a rodent cell, e.g., a rat or mouse) may be measured by fluorescent techniques (e.g., fluorescent microscopy or FACS) or light production in a luminometer (e.g., a spectrophotometer or an IVIS imaging instrument). In some embodiments, a reporter sequence can be used to verify the tissue-specific targeting capabilities and tissue-specific promoter regulatory and/or control activity of any of the constructs described herein.

In some embodiments, a reporter sequence is a FLAG tag (e.g., a 3xFLAG tag), and the presence of a construct carrying the FLAG tag in a non-human cell (e.g., a rodent cell, e.g., a rat or mouse) is detected by protein binding or detection assays (e.g., Western blots, immunohistochemistry, radioimmunoassay (RIA), mass spectrometry). An exemplary 3xFLAG tag sequence is provided as SEQ ID NO: 61.

SEQ ID NO: 61 - Exemplary 3xFLAG tag sequence

GGATCCCGGGCTGACTACAAAGACCATGACGGTGATTATAAAGATCATGA
CATCGACTACAAGGATGACGATGACAAG

### Exemplary Additional Sequences

In some embodiments, constructs of the present disclosure may include one or more cloning sites. In some such embodiments, cloning sites may not be fully removed prior to manufacturing for administration of a nucleic acid construct to a subject. In some embodiments, cloning sites may have functional roles including as linker sequences, or as portions of a Kozak site. As will be appreciated by those skilled in the art, cloning sites may vary significantly in primary sequence while retaining their desired function. In some embodiments, constructs may contain any combination of cloning sites. Certain cloning sites are presented below.

Exemplary cloning site A

TTAATTAA

Exemplary cloning site B

GCTAGCTCAGATCAGGCCGGCCCTATCTGCACGTGCGTACAGCGATCGCC
AAATTCGATATCAAGCTTATCGAT (SEQ ID NO: 94)

Exemplary cloning site C

ATCGATACCGTCGACCTCGACCTCGA (SEQ ID NO: 95)

Exemplary cloning site D

ACTAGTACGCGT (SEQ ID NO: 96)

### Targeting Vectors

Targeting vectors can be employed to introduce a nucleic acid construct into a target genomic locus. Targeting vectors can comprise a nucleic acid construct and homology arms that flank said nucleic acid construct; those skilled in the art will be aware of a variety of options and features generally applicable to the design, structure, and/or use of targeting vectors. For example, targeting vectors can be in linear form or in circular form, and they can be single-stranded or double-stranded. Targeting vectors can be deoxyribonucleic acid (DNA) or ribonucleic acid (RNA). For ease of reference, homology arms are referred to herein as 5′ and 3′ (i.e., upstream and downstream, i.e., left and right) homology arms. This terminology relates to the relative position of the homology arms to a nucleic acid construct within a targeting vector. 5′ and 3′ homology arms correspond to regions within a targeted locus or to a region within another targeting vector, which are referred to herein as “5′ target sequence” and “3′ target sequence,” respectively. In some embodiments, homology arms can also function as a 5′ or a 3′ target sequence. In some embodiments, the present disclosure provides targeting vectors comprising a provided technology whose sequence encodes an ADAR1 polypeptide or a characteristic portion thereof as described herein.

In some embodiments, methods described herein provide for traditional transgenic non-human animal creation. In such embodiments, a vector comprising an exogenous ADAR1 gene is injected into a zygote and integrated randomly within the genome. In some embodiments, such a random insertion site may be within a protein coding region and may result in the modification of function of an endogenous protein and/or gene. In some such embodiments, an exogenous ADAR1 gene may be incorporated as solely a coding region, as a coding region including a protein tag, as a coding region with an operably linked promoter, as a coding region including a poly(A) site, as a coding region including any additional regulatory region, or as any combination thereof.

In some embodiments, methods described herein provide for traditional transgenic non-human animal creation utilizing the Tol2 transposon system. In such embodiments, a vector comprising an exogenous ADAR1 gene is injected into a zygote and integrated randomly within an A/T rich region of the genome. In some embodiments, such a random insertion site may be within a protein coding region and may result in the modification of function of an endogenous protein and/or gene. In some such embodiments, an exogenous ADAR1 gene may be incorporated as solely a coding region, as a coding region including a protein tag, as a coding region with an operably linked promoter, as a coding region including a poly(A) site, as a coding region including any additional regulatory region, or as any combination thereof.

In some embodiments, methods described herein providing for traditional transgenic non-human animal creation may utilize large genomic fragments (e.g., 1mb, 10mb, 100mb, and/or 1000mb). In some embodiments, traditional transgenic non-human animals may comprise transgenic regions including promoters, introns, exons, and/or additional genomic regulatory regions. In some embodiments, traditional transgenic non-human animal creation may utilize a yeast artificial chromosome (YAC), a bacterial artificial chromosome (BAC), a human artificial chromosome, a P1-derived artificial chromosome (PAC), or any other engineered region which may be contained in an appropriate host cell.

In some embodiments, methods described herein employ two, three or more targeting vectors that are capable of recombining with each other. In some embodiments, first, second, and third targeting vectors each comprise a 5′ and a 3′ homology arm. The 3′ homology arm of the first targeting vector comprises a sequence that overlaps with the 5′ homology arm of the second targeting vector (i.e., overlapping sequences), which allows for homologous recombination between first and second vector.

In some embodiments of double component targeting methods, a 5′ homology arm of a first targeting vector and a 3′ homology arm of a second targeting vector can be similar to corresponding segments within a target genomic locus (i.e., a target sequence), which can promote homologous recombination of the first and the second targeting vectors with corresponding genomic segments and modify the target genomic locus.

In some embodiments of triple component targeting methods, a 3′ homology arm of a second targeting vector can comprise a sequence that overlaps with a 5′ homology arm of a third targeting vector (i.e., overlapping sequences), which can allow for homologous recombination between the second and the third targeting vector. The 5′ homology arm of the first targeting vector and the 3′ homology arm of the third targeting vector are similar to corresponding segments within the target genomic locus (i.e., the target sequence), which can promote homologous recombination of the first and the third targeting vectors with the corresponding genomic segments and modify the target genomic locus.

In some embodiments, a homology arm and a target sequence or two homology arms “correspond” or are “corresponding” to one another when the two regions share a sufficient level of sequence identity to one another so that they can act as substrates for a homologous recombination reaction. The sequence identity between a given target sequence and the corresponding homology arm found on a targeting vector (i.e., overlapping sequence) or between two homology arms can be any degree of sequence identity that allows for homologous recombination to occur. To give but one example, an amount of sequence identity shared by a homology arm of a targeting vector (or a fragment thereof) and a target sequence of another targeting vector or a target sequence of a target genomic locus (or a fragment thereof) can be, e.g., but not limited to, at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination.

Moreover, a corresponding region of similarity (e.g., identity) between a homology arm and a corresponding target sequence can be of any length that is sufficient to promote homologous recombination at the target genomic locus. For example, a given homology arm and/or corresponding target sequence can comprise corresponding regions of similarity that are but are not limited to, about 0.2-0.5 kb, 0.2-1 kb, 0.2-1.5 kb, 0.2-2 kb, 0.2-2.5 kb, 0.2-3 kb, 0.2-3.5 kb, 0.2-4 kb, 0.2-4.5 kb, or 0.2-5 kb in length such that a homology arm has sufficient similarity to undergo homologous recombination with a corresponding target sequence(s) within a target genomic locus of the cell or within another targeting vector. In some embodiments, a given homology arm and/or corresponding target sequence can comprise corresponding regions of similarity that are, e.g., but not limited to, about 5-10 kb, 5-15 kb, 5-20 kb, 5-25 kb, 5-30 kb, 5-35 kb, 5-40 kb, 5-45 kb, 5-50 kb, 5-55 kb, 5-60 kb, 5-65 kb, 5-70 kb, 5-75 kb, 5-80 kb, 5-85 kb, 5-90 kb, 5-95 kb, 5-100 kb, 100-200 kb, or 200-300 kb in length (such as described elsewhere herein) such that a homology arm has sufficient similarity to undergo homologous recombination with a corresponding target sequence(s) within a target genomic locus of the cell or within another targeting vector. In some embodiments, a given homology arm and/or corresponding target sequence comprise corresponding regions of similarity that are, e.g., but not limited to, about 10-100 kb, 15-100 kb, 20-100 kb, 25-100 kb, 30-100 kb, 35-100 kb, 40-100 kb, 45-100 kb, 50-100 kb, 55-100 kb, 60-100 kb, 65-100 kb, 70-100 kb, 75-100 kb, 80-100 kb, 85-100 kb, 90-100 kb, or 95-100 kb in length (such as described elsewhere herein) such that a homology arm has sufficient similarity to undergo homologous recombination with a corresponding target sequence(s) within a target genomic locus of the cell or within another targeting vector.

In some embodiments, overlapping sequences of a 3′ homology arm of a first targeting vector and a 5′ homology arm of a second targeting vector or of a 3′ homology arm of a second targeting vector and a 5′ homology arm of a third targeting vector can be of any length that is sufficient to promote homologous recombination between said targeting vectors. For example, a given homology arm and/or corresponding target sequence can comprise corresponding regions of similarity that are, e.g., but not limited to, about 0.2-0.5 kb, 0.2-1 kb, 0.2-1.5 kb, 0.2-2 kb, 0.2-2.5 kb, 0.2-3 kb, 0.2-3.5 kb, 0.2-4 kb, 0.2-4.5 kb, or 0.2-5 kb in length such that a homology arm has sufficient similarity to undergo homologous recombination with a corresponding target sequence(s) within a target genomic locus of the cell or within another targeting vector. In some embodiments, a given overlapping sequence of a homology arm can comprise corresponding overlapping regions that are about 1-5 kb, 5-10 kb, 5-15 kb, 5-20 kb, 5-25 kb, 5-30 kb, 5-35 kb, 5-40 kb, 5-45 kb, 5-50 kb, 5-55 kb, 5-60 kb, 5-65 kb, 5-70 kb, 5-75 kb, 5-80 kb, 5-85 kb, 5-90 kb, 5-95 kb, 5-100 kb, 100-200 kb, or 200-300 kb in length such that an overlapping sequence of a homology arm has sufficient similarity to undergo homologous recombination with a corresponding overlapping sequence within another targeting vector. In some embodiments, a given overlapping sequence of a homology arm comprises an overlapping region that is about 1-100 kb, 5-100 kb, 10-100 kb, 15-100 kb, 20-100 kb, 25-100 kb, 30-100 kb, 35-100 kb, 40-100 kb, 45-100 kb, 50-100 kb, 55-100 kb, 60-100 kb, 65-100 kb, 70-100 kb, 75-100 kb, 80-100 kb, 85-100 kb, 90-100 kb, or 95-100 kb in length such that an overlapping sequence of a homology arm has sufficient similarity to undergo homologous recombination with a corresponding overlapping sequence within another targeting vector. In some embodiments, an overlapping sequence is from 1-5 kb, inclusive. In some embodiments, an overlapping sequence is from about 1 kb to about 70 kb, inclusive. In some embodiments, an overlapping sequence is from about 10 kb to about 70 kb, inclusive. In some embodiments, an overlapping sequence is from about 10 kb to about 50 kb, inclusive. In some embodiments, an overlapping sequence is at least 10 kb. In some embodiments, an overlapping sequence is at least 20 kb. For example, an overlapping sequence can be from about 1 kb to about 5 kb, inclusive, from about 5 kb to about 10 kb, inclusive, from about 10 kb to about 15 kb, inclusive, from about 15 kb to about 20 kb, inclusive, from about 20 kb to about 25 kb, inclusive, from about 25 kb to about 30 kb, inclusive, from about 30 kb to about 35 kb, inclusive, from about 35 kb to about 40 kb, inclusive, from about 40 kb to about 45 kb, inclusive, from about 45 kb to about 50 kb, inclusive, from about 50 kb to about 60 kb, inclusive, from about 60 kb to about 70 kb, inclusive, from about 70 kb to about 80 kb, inclusive, from about 80 kb to about 90 kb, inclusive, from about 90 kb to about 100 kb, inclusive, from about 100 kb to about 120 kb, inclusive, from about 120 kb to about 140 kb, inclusive, from about 140 kb to about 160 kb, inclusive, from about 160 kb to about 180 kb, inclusive, from about 180 kb to about 200 kb, inclusive, from about 200 kb to about 220 kb, inclusive, from about 220 kb to about 240 kb, inclusive, from about 240 kb to about 260 kb, inclusive, from about 260 kb to about 280 kb, inclusive, or about 280 kb to about 300 kb, inclusive. To give but one example, an overlapping sequence can be from about 20 kb to about 60 kb, inclusive. Alternatively, an overlapping sequence can be at least 1 kb, at least 5 kb, at least 10 kb, at least 15 kb, at least 20 kb, at least 25 kb, at least 30 kb, at least 35 kb, at least 40 kb, at least 45 kb, at least 50 kb, at least 60 kb, at least 70 kb, at least 80 kb, at least 90 kb, at least 100 kb, at least 120 kb, at least 140 kb, at least 160 kb, at least 180 kb, at least 200 kb, at least 220 kb, at least 240 kb, at least 260 kb, at least 280 kb, or at least 300 kb. In some embodiments, an overlapping sequence can be at most 400 kb, at most 350 kb, at most 300 kb, at most 280 kb, at most 260 kb, at most 240 kb, at most 220 kb, at most 200 kb, at most 180 kb, at most 160 kb, at most 140 kb, at most 120 kb, at most 100 kb, at most 90 kb, at most 80 kb, at most 70 kb, at most 60 kb or at most 50 kb.

### Exemplary Sites for Genomic Incorporation

Homology arms can, in some embodiments, correspond to a locus that is native to a cell (e.g., a targeted locus), or alternatively they can correspond to a region of a heterologous or exogenous segment of DNA that was integrated into the genome of the cell, including, for example, transgenes, expression cassettes, or heterologous or exogenous regions of DNA. In some embodiments, homology arms can, correspond to a region on a targeting vector in a cell. In some embodiments, homology arms of a targeting vector may correspond to a region of a yeast artificial chromosome (YAC), a bacterial artificial chromosome (BAC), a human artificial chromosome, a P1-derived artificial chromosome (PAC), or any other engineered region contained in an appropriate host cell. Still further, homology arms of a targeting vector may correspond to or be derived from a region of a BAC library, a cosmid library, or a P1 phage library. In some certain embodiments, homology arms of a targeting vector correspond to a locus that is native, heterologous, or exogenous to a prokaryote, a yeast, a bird (e.g., chicken), a non-human mammal, a rodent, a human, a rat, a mouse, a hamster a rabbit, a pig, a bovine, a deer, a sheep, a goat, a cat, a dog, a ferret, a primate (e.g., marmoset, rhesus monkey), a domesticated mammal, an agricultural mammal, or any other organism of interest. In some embodiments, homology arms correspond to a locus of the cell that shows limited susceptibility to targeting using a conventional method or that has shown relatively low levels of successful integration at a targeted site, and/or significant levels of off-target integration, in the absence of a nick or double-strand break induced by a nuclease agent (e.g., a Cas protein, a Zinc Finger nuclease protein, and/or a TALEN protein). In some embodiments, homology arms are designed to include engineered DNA.

In some embodiments, 5′ and 3′ homology arms of a targeting vector(s) correspond to a targeted genome. Alternatively, homology arms correspond to a related genome. For example, a targeted genome is a mouse genome of a first strain, and targeting arms correspond to a mouse genome of a second strain, wherein the first strain and the second strain are different. In certain embodiments, homology arms correspond to the genome of the same animal or are from the genome of the same strain, e.g., the targeted genome is a mouse genome of a first strain, and the targeting arms correspond to a mouse genome from the same mouse or from the same strain.

A homology arm of a targeting vector can be of any length that is sufficient to promote a homologous recombination event with a corresponding target sequence, including, for example, 0.2-1 kb, inclusive, 1-5 kb, inclusive, 5-10 kb, inclusive, 5-15 kb, inclusive, 5-20 kb, inclusive, 5-25 kb, inclusive, 5-30 kb, inclusive, 5-35 kb, inclusive, 5-40 kb, inclusive, 5-45 kb, inclusive, 5-50 kb, inclusive, 5-55 kb, inclusive, 5-60 kb, inclusive, 5-65 kb, inclusive, 5-70 kb, inclusive, 5-75 kb, inclusive, 5-80 kb, inclusive, 5-85 kb, inclusive, 5-90 kb, inclusive, 5-95 kb, inclusive, 5-100 kb, inclusive, 100-200 kb, inclusive, or 200-300 kb, inclusive, in length. In some embodiments, a homology arm of a targeting vector has a length that is sufficient to promote a homologous recombination event with a corresponding target sequence that is 0.2-100 kb, inclusive, 1-100 kb, inclusive, 5-100 kb, inclusive, 10-100 kb, inclusive, 15-100 kb, inclusive, 20-100 kb, inclusive, 25-100 kb, inclusive, 30-100 kb, inclusive, 35-100 kb, inclusive, 40-100 kb, inclusive, 45-100 kb, inclusive, 50-100 kb, inclusive, 55-100 kb, inclusive, 60-100 kb, inclusive, 65-100 kb, inclusive, 70-100 kb, inclusive, 75-100 kb, inclusive, 80-100 kb, inclusive, 85-100 kb, inclusive, 90-100 kb, inclusive, or 95-100 kb, inclusive, in length. As described herein, large targeting vectors can employ targeting arms of greater length.

In certain embodiments, an ADAR1 polynucleotide or an exogenousADAR1 locus is incorporated into a non-human animal (e.g., a rodent, e.g., a rat or mouse) at an endogenous loci. In some cases, an endogenous loci is an ADAR1 locus. In some such cases, an endogenous ADAR1 locus may be replaced with an exogenous ADAR1 gene. In some embodiments, replacement may be partial, or may be complete. In some embodiments, an ADAR1 polynucleotide or an exogenous ADAR1 gene is incorporated into an endogenous ADAR1 locus and is operably linked to an endogenous ADAR1 promoter.

In certain embodiments, an ADAR1 polynucleotide or an exogenousADAR1 locus is incorporated into a non-human animal (e.g., a rodent, e.g., a rat or mouse) at an endogenous loci. In some cases, an endogenous loci is a locus driven by a constitutive promoter. In some embodiments, an endogenous loci is a locus driven by a tissue specific promoter.

In certain embodiments, an ADAR1 polynucleotide or an exogenous ADAR1 gene is incorporated into a non-human animal (e.g., a rodent, e.g., a rat or mouse) at a site amenable to Cre/LoxP manipulation. In certain embodiments, an ADAR1 polynucleotide or an exogenous ADAR1 is incorporated into a site or is located within a targeting vector flanked by LoxP recombination sites. In certain embodiments, a non-human animal (e.g., a rodent, e.g., a rat or mouse) with an exogenous ADAR1 gene comprising or incorporated into a site flanked by LoxP sites can further be crossed with an animal expressing a Cre recombinase under the control of one or more of a tissue specific, temporally specific, and/or inducible promoter.

In certain embodiments, an ADAR1 polynucleotide or an exogenous ADAR1 gene is incorporated into a non-human animal (e.g., rodent, e.g., rat or mouse), non-human (e.g., rodent, e.g., rat or mouse) cell or non-human (e.g., rodent, e.g., rat or mouse) tissue at a locus amenable for manipulation using Cre-Lox P and/or Flp-FRT; see E.g., Kim et al., “Mouse Cre-LoxP system: general principles to determine tissue-specific roles of target genes” Laboratory Animal Research (2018) 34(4), 147-159.

In certain embodiments, an ADAR1 polynucleotide or an exogenous ADAR1 gene is incorporated into a non-human animal (e.g., rodent, e.g., rat or mouse), non-human (e.g., rodent, e.g., rat or mouse) cell or non-human (e.g., rodent, e.g., rat or mouse) tissue at a Cre/LoxP stop or inducible Cre/LoxP site. In certain such embodiments, when crossed with a mouse that has Cre under a tissue specific promoter, said locus can generate tissue specific exogenous ADAR1 expression in transgenic animals.

In some embodiments of a non-human animal (e.g., rodent, e.g., rat or mouse), non-human (e.g., rodent, e.g., rat or mouse) cell or non-human (e.g., rodent, e.g., rat or mouse) tissue as described herein, the non-human animal, non-human cell or non-human tissue is homozygous or heterozygous for an exogenous ADAR1 gene integrated at a site operably linked to an inducible promoter (e.g., a tetracycline-responsive element, an estrogen receptor targeting motif, and/or under the control of tamoxifen).

In certain embodiments, an ADAR1 polynucleotide or an exogenous ADAR1 gene is incorporated into a non-human animal (e.g., rodent, e.g., rat or mouse), non-human (e.g., rodent, e.g., rat or mouse) cell or non-human (e.g., rodent, e.g., rat or mouse) tissue at a locus known to function as a transcriptional hotspot, and/or transcriptional safe harbor (such as are abundant and well known in the art).

In certain embodiments, an ADAR1 polynucleotide or an exogenous ADAR1 gene is incorporated into a non-human animal (e.g., rodent, e.g., rat or mouse), non-human (e.g., rodent, e.g., rat or mouse) cell or non-human (e.g., rodent, e.g., rat or mouse) tissue at the ROSA26 locus.

In certain embodiments, an ADAR1 polynucleotide or an exogenous ADAR1 gene is incorporated into a non-human animal (e.g., rodent, e.g., rat or mouse), non-human (e.g., rodent, e.g., rat or mouse) cell or non-human (e.g., rodent, e.g., rat or mouse) tissue at the H11 locus.

In certain embodiments, an ADAR1 polynucleotide or an exogenous ADAR1 gene is incorporated into a non-human animal (e.g., rodent, e.g., rat or mouse), non-human (e.g., rodent, e.g., rat or mouse) cell or non-human (e.g., rodent, e.g., rat or mouse) tissue at the TIGRE locus.

In certain embodiments, an ADAR1 polynucleotide or an exogenous ADAR1 gene is incorporated into a non-human animal (e.g., rodent, e.g., rat or mouse), non-human (e.g., rodent, e.g., rat or mouse) cell or non-human (e.g., rodent, e.g., rat or mouse) tissue at the MYH9 locus.

In some embodiments of a non-human animal (e.g., rodent, e.g., rat or mouse), non-human (e.g., rodent, e.g., rat or mouse) cell or non-human (e.g., rodent, e.g., rat or mouse) tissue as described herein, the non-human animal, non-human cell or non-human tissue is homozygous or heterozygous for an ADAR1 polynucleotide or an exogenous ADAR1 gene integrated at a site operably linked to a universally expressed promoter (e.g., CMV, SV40, elongation factor 1 alpha, CBA/CAGG, ubiquitin C, and/or phosphoglycerate kinase 1).

In certain embodiments, an ADAR1 polynucleotide or an ADAR1 locus is incorporated into a non-human animal (e.g., a rodent, e.g., a rat or mouse) at a ROSA26 locus site. In certain embodiments, a 5′ homology arm for insertion into a ROSA26 locus site comprises, or consists of, a nucleotide sequence that is the same as, differs by no more than 1, 2, 3, 4, 5, 6, 7, 8, 9, or 10 nucleotides from, or has at least has at least 90%, 92%, 94%, 96%, 98%, or 99% homology with, the nucleotide sequence of SEQ ID NO: 62. In some embodiments, a 5′ homology arm for insertion into a ROSA26 locus site is comprises, or consists of, a nucleotide sequence that is the same as the nucleotide sequence of SEQ ID NO: 62.

SEQ ID NO: 62 - Exemplary 5′ homology arm for insertion into a ROSA26 locus site

GGAAGAGTCCTGACCCAGGGAAGACATTAAAAAGGTAGTGGGGTCGACTA
GATGAAGGAGAGCCTTTCTCTCTGGGCAAGAGCGGTGCAATGGTGTGTAA
AGGTAGCTGAGAAGACGAAAAGGGCAAGCATCTTCCTGCTACCAGGCTGG
GGAGGCCCAGGCCCACGACCCCGAGGAGAGGGAACGCAGGGAGACTGAGG
TGACCCTTCTTTCCCCCGGGGCCCGGTCGTGTGGTTCGGTGTCTCTTTTC
TGTTGGACCCTTACCTTGACCCAGGCGCTGCCGGGGCCTGGGCCCGGGCT
GCGGCGCACGGCACTCCCGGGAGGCAGCGAGACTCGAGTTAGGCCCAACG
CGGCGCCACGGCGTTTCCTGGCCGGGAATGGCCCGTACCCGTGAGGTGGG
GGTGGGGGGCAGAAAAGGCGGAGCGAGCCCGAGGCGGGGAGGGGGAGGGC
CAGGGGCGGAGGGGGCCGGCACTACTGTGTTGGCGGACTGGCGGGACTAG
GGCTGCGTGAGTCTCTGAGCGCAGGCGGGCGGCGGCCGCCCCTCCCCCGG
CGGCGGCAGCGGCGGCAGCGGCGGCAGCTCACTCAGCCCGCTGCCCGAGC
GGAAACGCCACTGACCGCACGGGGATTCCCAGTGCCGGCGCCAGGGGCAC
GCGGGACACGCCCCCTCCCGCCGCGCCATTGGCCTCTCCGCCCACCGCCC
CACACTTATTGGCCGGTGCGCCGCCAATCAGCGGAGGCTGCCGGGGCCGC
CTAAAGAAGAGGCTGTGCTTTGGGGCTCCGGCTCCTCAGAGAGCCTCGGC
TAGGTAGGGGATCGGGACTCTGGCGGGAGGGCGGCTTGGTGCGTTTGCGG
GGATGGGCGGCCGCGGCAGGCCCTCCGAGCGTGGTGGAGCCGTTCTGTGA
GACAGCCGGGTACGAGTCGTGACGCTGGAAGGGGCAAGCGGGTGGTGGGC
AGGAATGCGGTCCGCCCTGCAGCAACCGGAGGGGGAGGGAGAAGGGAGCG
GAAAAGTCTCCACCGGACGCGGCCATGGCTCGGGGGGGGGGGGGCAGCGG
AGGAGCGCTTCCGGCCGACGTCTCGTCGCTGATTGGCTTCTTTTCCTCCC
GCCGTGTGTGAAAACACAAATGGCGTGTTTTGGTTGGCGTAAGGCGCCTG
TCAGTTAACGGCAGCCGGAGTGCGCAGCCGCCGGCAGCCTCGCTCTGCCC
ACTGGGTGGGGCGGGAGGTAGGTGGGGTGAGGCGAGCTGGACGTGCGGGC
GCGGTCGGCCTCTGGCGGGGCGGGGGAGGGGAGGGAGGGTCAGCGAAAGT
AGCTCGCGCGCGAGCGGCCGCCCACCCTCCCCTTCCTCTGGGGGAGTCGT
TTTACCCGCCGCCGGCCGGGCCTCGTCGTCTGATTGGCTCTCGGGGCCCA
GAAAACTGGCCCTTGCCATTGGCTCGTGTTCGTGCAAGTTGAGTCCATCC
GCCGGCCAGCGGGGGCGGCGAGGAGGCGCTCCCAGGTTCCGGCCCTCCCC
TCGGCCCCGCGCCGCAGAGTCTGGCCGCGCGCCCCTGCGCAACGTGGCAG
GAAGCGCGCGCTGGGGGCGGGGACGGGCAGTAGGGCTGAGCGGCTGCGGG
GCGGGTGCAAGCACGTTTCCGACTTGAGTTGCCTCAAGAGGGGCGTGCTG
AGCCAGACCTCCATCGCGCACTCCGGGGAGTGGAGGGAAGGAGCGAGGGC
TCAGTTGGGCTGTTTTGGAGGCAGGAAGCACTTGCTCTCCCAAAGTCGCT
CTGAGTTGTTATCAGTAAGGGAGCTGCAGTGGAGTAGGCGGGGAGAAGGC
CGCACCCTTCTC

In certain embodiments, an ADAR1 polynucleotide or an ADAR1 locus is incorporated into a non-human animal (e.g., a rodent, e.g., a rat or mouse) at a ROSA26 locus site. In certain embodiments, a 3′ homology arm for insertion into a ROSA26 locus site comprises, or consists of, a nucleotide sequence that is the same as, differs by no more than 1, 2, 3, 4, 5, 6, 7, 8, 9, or 10 nucleotides from, or has at least has at least 90%, 92%, 94%, 96%, 98%, or 99% homology with, the nucleotide sequence of SEQ ID NO: 63. In some embodiments, a 3′ homology arm for insertion into a ROSA26 locus site is comprises, or consists of, a nucleotide sequence that is the same as the nucleotide sequence of SEQ ID NO: 63.

SEQ ID NO: 63 - Exemplary 3′ homology arm for insertion into a ROSA26 locus site

CGGAGGGGGGAGGGGAGTGTTGCAATACCTTTCTGGGAGTTCTCTGCTGC
CTCCTGGCTTCTGAGGACCGCCCTGGGCCTGGGAGAATCCCTTCCCCCTC
TTCCCTCGTGATCTGCAACTCCAGTCTTTCTAGAAGATGGGCGGGAGTCT
TCTGGGCAGGCTTAAAGGCTAACCTGGTGTGTGGGCGTTGTCCTGCAGGG
GAATTGAACAGGTGTAAAATTGGAGGGACAAGACTTCCCACAGATTTTCG
GTTTTGTCGGGAAGTTTTTTAATAGGGGCAAATAAGGAAAATGGGAGGAT
AGGTAGTCATCTGGGGTTTTATGCAGCAAAACTACAGGTTATTATTGCTT
GTGATCCGCCTCGGAGTATTTTCCATCGAGGTAGATTAAAGACATGCTCA
CCCGAGTTTTATACTCTCCTGCTTGAGATCCTTACTACAGTATGAAATTA
CAGTGTCGCGAGTTAGACTATGTAAGCAGAATTTTAATCATTTTTAAAGA
GCCCAGTACTTCATATCCATTTCTCCCGCTCCTTCTGCAGCCTTATCAAA
AGGTATTTTAGAACACTCATTTTAGCCCCATTTTCATTTATTATACTGGC
TTATCCAACCCCTAGACAGAGCATTGGCATTTTCCCTTTCCTGATCTTAG
AAGTCTGATGACTCATGAAACCAGACAGATTAGTTACATACACCACAAAT
CGAGGCTGTAGCTGGGGCCTCAACACTGCAGTTCTTTTATAACTCCTTAG
TACACTTTTTGTTGATCCTTTGCCTTGATCCTTAATTTTCAGTGTCTATC
ACCTCTCCCGTCAGGTGGTGTTCCACATTTGGGCCTATTCTCAGTCCAGG
GAGTTTTACAACAATAGATGTATTGAGAATCCAACCTAAAGCTTAACTTT
CCACTCCCATGAATGCCTCTCTCCTTTTTCTCCATTTATAAACTGAGCTA
TTAACCATTAATGGTTTCCAGGTGGATGTCTCCTCCCCCAATATTACCTG
ATGTATCTTACATATTGCCAGGCTGATATTTTAAGACATTAAAAGGTATA
TTTCATTATTGAGCCACATGGTATTGATTACTGCTTACTAAAATTTTGTC
ATTGTACACATCTGTAAAAGGTGGTTCCTTTTGGAATGCAAAGTTCAGGT
GTTTGTTGTCTTTCCTGACCTAAGGTCTTGTGAGCTTGTATTTTTTCTAT
TTAAGCAGTGCTTTCTCTTGGACTGGCTTGACTCATGGCATTCTACACGT
TATTGCTGGTCTAAATGTGATTTTGCCAAGCTTCTTCAGGACCTATAATT
TTGCTTGACTTGTAGCCAAACACAAGTAAAATGATTAAGCAACAAATGTA
TTTGTGAAGCTTGGTTTTTAGGTTGTTGTGTTGTGTGTGCTTGTGCTCTA
TAATAATACTATCCAGGGGCTGGAGAGGTGGCTCGGAGTTCAAGAGCACA
GACTGCTCTTCCAGAAGTCCTGAGTTCAATTCCCAGCAACCACATGGTGG
CTCACAACCATCTGTAATGGGATCTGATGCCCTCTTCTGGTGTGTCTGAA
GACCACAAGTGTATTCACATTAAATAAATAAATCCTCCTTCTTCTTCTTT
TTTTTTTTTTTAAAGAGAATACTGTCTCCAGTAGAATTTACTGAAGTAAT
GAAATACTTTGTGTTTGTTCCAATATGGTAGCCAATAATCAAATTACTCT
TTAAGCACTGGAAATGTTACCAAGGAACTAATTTTTATTTGAAGTGTAAC
TGTGGACAGAGGAGCCATAACTGCAGACT

### Exemplary Targeting Vectors for Genomic Incorporation

In certain embodiments, an ADAR1 polynucleotide or an ADAR1 locus is incorporated into a non-human animal (e.g., a rodent, e.g., a rat or mouse) at a ROSA26 locus site utilizing a targeting vector. In certain embodiments, a targeting vector for insertion into a ROSA26 locus site comprises, or consists of, a nucleotide sequence that is the same as, or has at least has at least 60%, 65%, 70%, 75%, 80%, 82%, 84%, 86%, 88%, 90%, 92%, 94%, 96%, 98%, or 99% homology with, the nucleotide sequence of SEQ ID NO: 64. In certain embodiments, a targeting vector for insertion into a ROSA26 locus site comprises, or consists of, a nucleotide sequence that is the same as the nucleotide sequence of SEQ ID NO: 64.

SEQ ID NO: 64 - Exemplary ADAR1 targeting vector for insertion into a ROSA26 locus

GGAAGAGTCCTGACCCAGGGAAGACATTAAAAAGGTAGTGGGGTCGACTA
GATGAAGGAGAGCCTTTCTCTCTGGGCAAGAGCGGTGCAATGGTGTGTAA
AGGTAGCTGAGAAGACGAAAAGGGCAAGCATCTTCCTGCTACCAGGCTGG
GGAGGCCCAGGCCCACGACCCCGAGGAGAGGGAACGCAGGGAGACTGAGG
TGACCCTTCTTTCCCCCGGGGCCCGGTCGTGTGGTTCGGTGTCTCTTTTC
TGTTGGACCCTTACCTTGACCCAGGCGCTGCCGGGGCCTGGGCCCGGGCT
GCGGCGCACGGCACTCCCGGGAGGCAGCGAGACTCGAGTTAGGCCCAACG
CGGCGCCACGGCGTTTCCTGGCCGGGAATGGCCCGTACCCGTGAGGTGGG
GGTGGGGGGCAGAAAAGGCGGAGCGAGCCCGAGGCGGGGAGGGGGAGGGC
CAGGGGCGGAGGGGGCCGGCACTACTGTGTTGGCGGACTGGCGGGACTAG
GGCTGCGTGAGTCTCTGAGCGCAGGCGGGCGGCGGCCGCCCCTCCCCCGG
CGGCGGCAGCGGCGGCAGCGGCGGCAGCTCACTCAGCCCGCTGCCCGAGC
GGAAACGCCACTGACCGCACGGGGATTCCCAGTGCCGGCGCCAGGGGCAC
GCGGGACACGCCCCCTCCCGCCGCGCCATTGGCCTCTCCGCCCACCGCCC
CACACTTATTGGCCGGTGCGCCGCCAATCAGCGGAGGCTGCCGGGGCCGC
CTAAAGAAGAGGCTGTGCTTTGGGGCTCCGGCTCCTCAGAGAGCCTCGGC
TAGGTAGGGGATCGGGACTCTGGCGGGAGGGCGGCTTGGTGCGTTTGCGG
GGATGGGCGGCCGCGGCAGGCCCTCCGAGCGTGGTGGAGCCGTTCTGTGA
GACAGCCGGGTACGAGTCGTGACGCTGGAAGGGGCAAGCGGGTGGTGGGC
AGGAATGCGGTCCGCCCTGCAGCAACCGGAGGGGGAGGGAGAAGGGAGCG
GAAAAGTCTCCACCGGACGCGGCCATGGCTCGGGGGGGGGGGGGCAGCGG
AGGAGCGCTTCCGGCCGACGTCTCGTCGCTGATTGGCTTCTTTTCCTCCC
GCCGTGTGTGAAAACACAAATGGCGTGTTTTGGTTGGCGTAAGGCGCCTG
TCAGTTAACGGCAGCCGGAGTGCGCAGCCGCCGGCAGCCTCGCTCTGCCC
ACTGGGTGGGGCGGGAGGTAGGTGGGGTGAGGCGAGCTGGACGTGCGGGC
GCGGTCGGCCTCTGGCGGGGCGGGGGAGGGGAGGGAGGGTCAGCGAAAGT
AGCTCGCGCGCGAGCGGCCGCCCACCCTCCCCTTCCTCTGGGGGAGTCGT
TTTACCCGCCGCCGGCCGGGCCTCGTCGTCTGATTGGCTCTCGGGGCCCA
GAAAACTGGCCCTTGCCATTGGCTCGTGTTCGTGCAAGTTGAGTCCATCC
GCCGGCCAGCGGGGGCGGCGAGGAGGCGCTCCCAGGTTCCGGCCCTCCCC
TCGGCCCCGCGCCGCAGAGTCTGGCCGCGCGCCCCTGCGCAACGTGGCAG
GAAGCGCGCGCTGGGGGCGGGGACGGGCAGTAGGGCTGAGCGGCTGCGGG
GCGGGTGCAAGCACGTTTCCGACTTGAGTTGCCTCAAGAGGGGCGTGCTG
AGCCAGACCTCCATCGCGCACTCCGGGGAGTGGAGGGAAGGAGCGAGGGC
TCAGTTGGGCTGTTTTGGAGGCAGGAAGCACTTGCTCTCCCAAAGTCGCT
CTGAGTTGTTATCAGTAAGGGAGCTGCAGTGGAGTAGGCGGGGAGAAGGC
CGCACCCTTCTCTTAATTAAGTGACCTGCACGTCTAGGGCGCAGTAGTCC
AGGGTTTCCTTGATGATGTCATACTTATCCTGTCCCTTTTTTTTCCACAG
CTCGCGGTTGAGGACAAACTCTTCGCGGTCTTTCCAGTGCCACCATGGCC
GAGATCAAGGAGAAAATCTGCGACTATCTCTTCAATGTGTCTGACTCCTC
TGCCCTGAATTTGGCTAAAAATATTGGCCTTACCAAGGCCCGAGATATAA
ATGCTGTGCTAATTGACATGGAAAGGCAGGGGGATGTCTATAGACAAGGG
ACAACCCCTCCCATATGGCATTTGACAGACAAGAAGCGAGAGAGGATGCA
AATCAAGAGAAATACGAACAGTGTTCCTGAAACCGCTCCAGCTGCAATCC
CTGAGACCAAAAGAAACGCAGAGTTCCTCACCTGTAATATACCCACATCA
AATGCCTCAAATAACATGGTAACCACAGAAAAAGTGGAGAATGGGCAGGA
ACCTGTCATAAAGTTAGAAAACAGGCAAGAGGCCAGACCAGAACCAGCAA
GACTGAAACCACCTGTTCATTACAATGGCCCCTCAAAAGCAGGGTATGTT
GACTTTGAAAATGGCCAGTGGGCCACAGATGACATCCCAGATGACTTGAA
TAGTATCCGCGCAGCACCAGGTGAGTTTCGAGCCATCATGGAGATGCCCT
CCTTCTACAGTCATGGCTTGCCACGGTGTTCACCCTACAAGAAACTGACA
GAGTGCCAGCTGAAGAACCCCATCAGCGGGCTGTTAGAATATGCCCAGTT
CGCTAGTCAAACCTGTGAGTTCAACATGATAGAGCAGAGTGGACCACCCC
ATGAACCTCGATTTAAATTCCAGGTTGTCATCAATGGCCGAGAGTTTCCC
CCAGCTGAAGCTGGAAGCAAGAAAGTGGCCAAGCAGGATGCAGCTATGAA
AGCCATGACAATTCTGCTAGAGGAAGCCAAAGCCAAGGACAGTGGAAAAT
CAGAAGAATCATCCCACTATTCCACAGAGAAAGAATCAGAGAAGACTGCA
GAGTCCCAGACCCCCACCCCTTCAGCCACATCCTTCTTTTCTGGGAAGAG
CCCCGTCACCACACTGCTTGAGTGTATGCACAAATTGGGGAACTCCTGCG
AATTCCGTCTCCTGTCCAAAGAAGGCCCTGCCCATGAACCCAAGTTCCAA
TACTGTGTTGCAGTGGGAGCCCAAACTTTCCCCAGTGTGAGTGCTCCCAG
CAAGAAAGTGGCAAAGCAGATGGCCGCAGAGGAAGCCATGAAGGCCCTGC
ATGGGGAGGCGACCAACTCCATGGCTTCTGATAACCAGCCTGAAGGTATG
ATCTCAGAGTCACTTGATAACTTGGAATCCATGATGCCCAACAAGGTCAG
GAAGATTGGCGAGCTCGTGAGATACCTGAACACCAACCCTGTGGGTGGCC
TTTTGGAGTACGCCCGCTCCCATGGCTTTGCTGCTGAATTCAAGTTGGTC
GACCAGTCCGGACCTCCTCACGAGCCCAAGTTCGTTTACCAAGCAAAAGT
TGGGGGTCGCTGGTTCCCAGCCGTCTGCGCACACAGCAAGAAGCAAGGCA
AGCAGGAAGCAGCAGATGCGGCTCTCCGTGTCTTGATTGGGGAGAACGAG
AAGGCAGAACGCATGGGTTTCACAGAGGTAACCCCAGTGACAGGGGCCAG
TCTCAGAAGAACTATGCTCCTCCTCTCAAGGTCCCCAGAAGCACAGCCAA
AGACACTCCCTCTCACTGGCAGCACCTTCCATGACCAGATAGCCATGCTG
AGCCACCGGTGCTTCAACACTCTGACTAACAGCTTCCAGCCCTCCTTGCT
CGGCCGCAAGATTCTGGCCGCCATCATTATGAAAAAAGACTCTGAGGACA
TGGGTGTCGTCGTCAGCTTGGGAACAGGGAATCGCTGTGTAAAAGGAGAT
TCTCTCAGCCTAAAAGGAGAAACTGTCAATGACTGCCATGCAGAAATAAT
CTCCCGGAGAGGCTTCATCAGGTTTCTCTACAGTGAGTTAATGAAATACA
ACTCCCAGACTGCGAAGGATAGTATATTTGAACCTGCTAAGGGAGGAGAA
AAGCTCCAAATAAAAAAGACTGTGTCATTCCATCTGTATATCAGCACTGC
TCCGTGTGGAGATGGCGCCCTCTTTGACAAGTCCTGCAGCGACCGTGCTA
TGGAAAGCACAGAATCCCGCCACTACCCTGTCTTCGAGAATCCCAAACAA
GGAAAGCTCCGCACCAAGGTGGAGAACGGAGAAGGCACAATCCCTGTGGA
ATCCAGTGACATTGTGCCTACGTGGGATGGCATTCGGCTCGGGGAGAGAC
TCCGTACCATGTCCTGTAGTGACAAAATCCTACGCTGGAACGTGCTGGGC
CTGCAAGGGGCACTGTTGACCCACTTCCTGCAGCCCATTTATCTCAAATC
TGTCACATTGGGTTACCTTTTCAGCCAAGGGCATCTGACCCGTGCTATTT
GCTGTCGTGTGACAAGAGATGGGAGTGCATTTGAGGATGGACTACGACAT
CCCTTTATTGTCAACCACCCCAAGGTTGGCAGAGTCAGCATATATGATTC
CAAAAGGCAATCCGGGAAGACTAAGGAGACAAGCGTCAACTGGTGTCTGG
CTGATGGCTATGACCTGGAGATCCTGGACGGTACCAGAGGCACTGTGGAT
GGGCCACGGAATGAATTGTCCCGGGTCTCCAAAAAGAACATTTTTCTTCT
ATTTAAGAAGCTCTGCTCCTTCCGTTACCGCAGGGATCTACTGAGACTCT
CCTATGGTGAGGCCAAGAAAGCTGCCCGTGACTACGAGACGGCCAAGAAC
TACTTCAAAAAAGGCCTGAAGGATATGGGCTATGGGAACTGGATTAGCAA
ACCCCAGGAGGAAAAGAACTTTTATCTCTGCCCAGTATAGGCTAGCTCAG
ATCAGGCCGGCCCTATCTGCACGTGCGTACAGCGATCGCCAAATTCGATA
TCAAGCTTATCGATAATCAACCTCTGGATTACAAAATTTGTGAAAGATTG
ACTGGTATTCTTAACTATGTTGCTCCTTTTACGCTATGTGGATACGCTGC
TTTAATGCCTTTGTATCATGCTATTGCTTCCCGTATGGCTTTCATTTTCT
CCTCCTTGTATAAATCCTGGTTGCTGTCTCTTTATGAGGAGTTGTGGCCC
GTTGTCAGGCAACGTGGCGTGGTGTGCACTGTGTTTGCTGACGCAACCCC
CACTGGTTGGGGCATTGCCACCACCTGTCAGCTCCTTTCCGGGACTTTCG
CTTTCCCCCTCCCTATTGCCACGGCGGAACTCATCGCCGCCTGCCTTGCC
CGCTGCTGGACAGGGGCTCGGCTGTTGGGCACTGACAATTCCGTGGTGTT
GTCGGGGAAATCATCGTCCTTTCCTTGGCTGCTCGCCTGTGTTGCCACCT
GGATTCTGCGCGGGACGTCCTTCTGCTACGTCCCTTCGGCCCTCAATCCA
GCGGACCTTCCTTCCCGCGGCCTGCTGCCGGCTCTGCGGCCTCTTCCGCG
TCTTCGCCTTCGCCCTCAGACGAGTCGGATCTCCCTTTGGGCCGCCTCCC
CGCATCGATACCGTCGACCTCGACCTCGACTGTGCCTTCTAGTTGCCAGC
CATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCT
GAGTAGGTGTCATTCTATTCTGGGGGGTGGGGTGGGGCAGGACAGCAAGG
GGGAGGATTGGGAAGACAATGGCAGGCATGCTGGGGAACTAGTACGCGTC
GGAGGGGGGAGGGGAGTGTTGCAATACCTTTCTGGGAGTTCTCTGCTGCC
TCCTGGCTTCTGAGGACCGCCCTGGGCCTGGGAGAATCCCTTCCCCCTCT
TCCCTCGTGATCTGCAACTCCAGTCTTTCTAGAAGATGGGCGGGAGTCTT
CTGGGCAGGCTTAAAGGCTAACCTGGTGTGTGGGCGTTGTCCTGCAGGGG
AATTGAACAGGTGTAAAATTGGAGGGACAAGACTTCCCACAGATTTTCGG
TTTTGTCGGGAAGTTTTTTAATAGGGGCAAATAAGGAAAATGGGAGGATA
GGTAGTCATCTGGGGTTTTATGCAGCAAAACTACAGGTTATTATTGCTTG
TGATCCGCCTCGGAGTATTTTCCATCGAGGTAGATTAAAGACATGCTCAC
CCGAGTTTTATACTCTCCTGCTTGAGATCCTTACTACAGTATGAAATTAC
AGTGTCGCGAGTTAGACTATGTAAGCAGAATTTTAATCATTTTTAAAGAG
CCCAGTACTTCATATCCATTTCTCCCGCTCCTTCTGCAGCCTTATCAAAA
GGTATTTTAGAACACTCATTTTAGCCCCATTTTCATTTATTATACTGGCT
TATCCAACCCCTAGACAGAGCATTGGCATTTTCCCTTTCCTGATCTTAGA
AGTCTGATGACTCATGAAACCAGACAGATTAGTTACATACACCACAAATC
GAGGCTGTAGCTGGGGCCTCAACACTGCAGTTCTTTTATAACTCCTTAGT
ACACTTTTTGTTGATCCTTTGCCTTGATCCTTAATTTTCAGTGTCTATCA
CCTCTCCCGTCAGGTGGTGTTCCACATTTGGGCCTATTCTCAGTCCAGGG
AGTTTTACAACAATAGATGTATTGAGAATCCAACCTAAAGCTTAACTTTC
CACTCCCATGAATGCCTCTCTCCTTTTTCTCCATTTATAAACTGAGCTAT
TAACCATTAATGGTTTCCAGGTGGATGTCTCCTCCCCCAATATTACCTGA
TGTATCTTACATATTGCCAGGCTGATATTTTAAGACATTAAAAGGTATAT
TTCATTATTGAGCCACATGGTATTGATTACTGCTTACTAAAATTTTGTCA
TTGTACACATCTGTAAAAGGTGGTTCCTTTTGGAATGCAAAGTTCAGGTG
TTTGTTGTCTTTCCTGACCTAAGGTCTTGTGAGCTTGTATTTTTTCTATT
TAAGCAGTGCTTTCTCTTGGACTGGCTTGACTCATGGCATTCTACACGTT
ATTGCTGGTCTAAATGTGATTTTGCCAAGCTTCTTCAGGACCTATAATTT
TGCTTGACTTGTAGCCAAACACAAGTAAAATGATTAAGCAACAAATGTAT
TTGTGAAGCTTGGTTTTTAGGTTGTTGTGTTGTGTGTGCTTGTGCTCTAT
AATAATACTATCCAGGGGCTGGAGAGGTGGCTCGGAGTTCAAGAGCACAG
ACTGCTCTTCCAGAAGTCCTGAGTTCAATTCCCAGCAACCACATGGTGGC
TCACAACCATCTGTAATGGGATCTGATGCCCTCTTCTGGTGTGTCTGAAG
ACCACAAGTGTATTCACATTAAATAAATAAATCCTCCTTCTTCTTCTTTT
TTTTTTTTTTAAAGAGAATACTGTCTCCAGTAGAATTTACTGAAGTAATG
AAATACTTTGTGTTTGTTCCAATATGGTAGCCAATAATCAAATTACTCTT
TAAGCACTGGAAATGTTACCAAGGAACTAATTTTTATTTGAAGTGTAACT
GTGGACAGAGGAGCCATAACTGCAGACT

In certain embodiments, an ADAR1 polynucleotide or an ADAR1 locus is incorporated into a non-human animal (e.g., a rodent, e.g., a rat or mouse) at a ROSA26 locus site utilizing a targeting vector. In certain embodiments, a targeting vector for insertion into a ROSA26 locus site comprises, or consists of, a nucleotide sequence that is the same as, or has at least has at least 60%, 65%, 70%, 75%, 80%, 82%, 84%, 86%, 88%, 90%, 92%, 94%, 96%, 98%, or 99% homology with, the nucleotide sequence of SEQ ID NO: 65. In certain embodiments, a targeting vector for insertion into a ROSA26 locus site comprises, or consists of, a nucleotide sequence that is the same as the nucleotide sequence of SEQ ID NO: 65.

SEQ ID NO: 65 - Exemplary ADAR1 targeting vector for insertion into a ROSA26 locus

GGAAGAGTCCTGACCCAGGGAAGACATTAAAAAGGTAGTGGGGTCGACTA
GATGAAGGAGAGCCTTTCTCTCTGGGCAAGAGCGGTGCAATGGTGTGTAA
AGGTAGCTGAGAAGACGAAAAGGGCAAGCATCTTCCTGCTACCAGGCTGG
GGAGGCCCAGGCCCACGACCCCGAGGAGAGGGAACGCAGGGAGACTGAGG
TGACCCTTCTTTCCCCCGGGGCCCGGTCGTGTGGTTCGGTGTCTCTTTTC
TGTTGGACCCTTACCTTGACCCAGGCGCTGCCGGGGCCTGGGCCCGGGCT
GCGGCGCACGGCACTCCCGGGAGGCAGCGAGACTCGAGTTAGGCCCAACG
CGGCGCCACGGCGTTTCCTGGCCGGGAATGGCCCGTACCCGTGAGGTGGG
GGTGGGGGGCAGAAAAGGCGGAGCGAGCCCGAGGCGGGGAGGGGGAGGGC
CAGGGGCGGAGGGGGCCGGCACTACTGTGTTGGCGGACTGGCGGGACTAG
GGCTGCGTGAGTCTCTGAGCGCAGGCGGGCGGCGGCCGCCCCTCCCCCGG
CGGCGGCAGCGGCGGCAGCGGCGGCAGCTCACTCAGCCCGCTGCCCGAGC
GGAAACGCCACTGACCGCACGGGGATTCCCAGTGCCGGCGCCAGGGGCAC
GCGGGACACGCCCCCTCCCGCCGCGCCATTGGCCTCTCCGCCCACCGCCC
CACACTTATTGGCCGGTGCGCCGCCAATCAGCGGAGGCTGCCGGGGCCGC
CTAAAGAAGAGGCTGTGCTTTGGGGCTCCGGCTCCTCAGAGAGCCTCGGC
TAGGTAGGGGATCGGGACTCTGGCGGGAGGGCGGCTTGGTGCGTTTGCGG
GGATGGGCGGCCGCGGCAGGCCCTCCGAGCGTGGTGGAGCCGTTCTGTGA
GACAGCCGGGTACGAGTCGTGACGCTGGAAGGGGCAAGCGGGTGGTGGGC
AGGAATGCGGTCCGCCCTGCAGCAACCGGAGGGGGAGGGAGAAGGGAGCG
GAAAAGTCTCCACCGGACGCGGCCATGGCTCGGGGGGGGGGGGGCAGCGG
AGGAGCGCTTCCGGCCGACGTCTCGTCGCTGATTGGCTTCTTTTCCTCCC
GCCGTGTGTGAAAACACAAATGGCGTGTTTTGGTTGGCGTAAGGCGCCTG
TCAGTTAACGGCAGCCGGAGTGCGCAGCCGCCGGCAGCCTCGCTCTGCCC
ACTGGGTGGGGCGGGAGGTAGGTGGGGTGAGGCGAGCTGGACGTGCGGGC
GCGGTCGGCCTCTGGCGGGGCGGGGGAGGGGAGGGAGGGTCAGCGAAAGT
AGCTCGCGCGCGAGCGGCCGCCCACCCTCCCCTTCCTCTGGGGGAGTCGT
TTTACCCGCCGCCGGCCGGGCCTCGTCGTCTGATTGGCTCTCGGGGCCCA
GAAAACTGGCCCTTGCCATTGGCTCGTGTTCGTGCAAGTTGAGTCCATCC
GCCGGCCAGCGGGGGCGGCGAGGAGGCGCTCCCAGGTTCCGGCCCTCCCC
TCGGCCCCGCGCCGCAGAGTCTGGCCGCGCGCCCCTGCGCAACGTGGCAG
GAAGCGCGCGCTGGGGGCGGGGACGGGCAGTAGGGCTGAGCGGCTGCGGG
GCGGGTGCAAGCACGTTTCCGACTTGAGTTGCCTCAAGAGGGGCGTGCTG
AGCCAGACCTCCATCGCGCACTCCGGGGAGTGGAGGGAAGGAGCGAGGGC
TCAGTTGGGCTGTTTTGGAGGCAGGAAGCACTTGCTCTCCCAAAGTCGCT
CTGAGTTGTTATCAGTAAGGGAGCTGCAGTGGAGTAGGCGGGGAGAAGGC
CGCACCCTTCTCTTAATTAAGTGACCTGCACGTCTAGGGCGCAGTAGTCC
AGGGTTTCCTTGATGATGTCATACTTATCCTGTCCCTTTTTTTTCCACAG
CTCGCGGTTGAGGACAAACTCTTCGCGGTCTTTCCAGTGCCACCATGAAT
CCGCGGCAGGGGTATTCCCTCAGCGGATACTACACCCATCCATTTCAAGG
CTATGAGCACAGACAGCTCAGATACCAGCAGCCTGGGCCAGGATCTTCCC
CCAGTAGTTTCCTGCTTAAGCAAATAGAATTTCTCAAGGGGCAGCTCCCA
GAAGCACCGGTGATTGGAAAGCAGACACCGTCACTGCCACCTTCCCTCCC
AGGACTCCGGCCAAGGTTTCCAGTACTACTTGCCTCCAGTACCAGAGGCA
GGCAAGTGGACATCAGGGGTGTCCCCAGGGGCGTGCATCTCGGAAGTCAG
GGGCTCCAGAGAGGGTTCCAGCATCCTTCACCACGTGGCAGGAGTCTGCC
ACAGAGAGGTGTTGATTGCCTTTCCTCACATTTCCAGGAACTGAGTATCT
ACCAAGATCAGGAACAAAGGATCTTAAAGTTCCTGGAAGAGCTTGGGGAA
GGGAAGGCCACCACAGCACATGATCTGTCTGGGAAACTTGGGACTCCGAA
GAAAGAAATCAATCGAGTTTTATACTCCCTGGCAAAGAAGGGCAAGCTAC
AGAAAGAGGCAGGAACACCCCCTTTGTGGAAAATCGCGGTCTCCACTCAG
GCTTGGAACCAGCACAGCGGAGTGGTAAGACCAGACGGTCATAGCCAAGG
AGCCCCAAACTCAGACCCGAGTTTGGAACCGGAAGACAGAAACTCCACAT
CTGTCTCAGAAGATCTTCTTGAGCCTTTTATTGCAGTCTCAGCTCAGGCT
TGGAACCAGCACAGCGGAGTGGTAAGACCAGACAGTCATAGCCAAGGATC
CCCAAACTCAGACCCAGGTTTGGAACCTGAAGACAGCAACTCCACATCTG
CCTTGGAAGATCCTCTTGAGTTTTTAGACATGGCCGAGATCAAGGAGAAA
ATCTGCGACTATCTCTTCAATGTGTCTGACTCCTCTGCCCTGAATTTGGC
TAAAAATATTGGCCTTACCAAGGCCCGAGATATAAATGCTGTGCTAATTG
ACATGGAAAGGCAGGGGGATGTCTATAGACAAGGGACAACCCCTCCCATA
TGGCATTTGACAGACAAGAAGCGAGAGAGGATGCAAATCAAGAGAAATAC
GAACAGTGTTCCTGAAACCGCTCCAGCTGCAATCCCTGAGACCAAAAGAA
ACGCAGAGTTCCTCACCTGTAATATACCCACATCAAATGCCTCAAATAAC
ATGGTAACCACAGAAAAAGTGGAGAATGGGCAGGAACCTGTCATAAAGTT
AGAAAACAGGCAAGAGGCCAGACCAGAACCAGCAAGACTGAAACCACCTG
TTCATTACAATGGCCCCTCAAAAGCAGGGTATGTTGACTTTGAAAATGGC
CAGTGGGCCACAGATGACATCCCAGATGACTTGAATAGTATCCGCGCAGC
ACCAGGTGAGTTTCGAGCCATCATGGAGATGCCCTCCTTCTACAGTCATG
GCTTGCCACGGTGTTCACCCTACAAGAAACTGACAGAGTGCCAGCTGAAG
AACCCCATCAGCGGGCTGTTAGAATATGCCCAGTTCGCTAGTCAAACCTG
TGAGTTCAACATGATAGAGCAGAGTGGACCACCCCATGAACCTCGATTTA
AATTCCAGGTTGTCATCAATGGCCGAGAGTTTCCCCCAGCTGAAGCTGGA
AGCAAGAAAGTGGCCAAGCAGGATGCAGCTATGAAAGCCATGACAATTCT
GCTAGAGGAAGCCAAAGCCAAGGACAGTGGAAAATCAGAAGAATCATCCC
ACTATTCCACAGAGAAAGAATCAGAGAAGACTGCAGAGTCCCAGACCCCC
ACCCCTTCAGCCACATCCTTCTTTTCTGGGAAGAGCCCCGTCACCACACT
GCTTGAGTGTATGCACAAATTGGGGAACTCCTGCGAATTCCGTCTCCTGT
CCAAAGAAGGCCCTGCCCATGAACCCAAGTTCCAATACTGTGTTGCAGTG
GGAGCCCAAACTTTCCCCAGTGTGAGTGCTCCCAGCAAGAAAGTGGCAAA
GCAGATGGCCGCAGAGGAAGCCATGAAGGCCCTGCATGGGGAGGCGACCA
ACTCCATGGCTTCTGATAACCAGCCTGAAGGTATGATCTCAGAGTCACTT
GATAACTTGGAATCCATGATGCCCAACAAGGTCAGGAAGATTGGCGAGCT
CGTGAGATACCTGAACACCAACCCTGTGGGTGGCCTTTTGGAGTACGCCC
GCTCCCATGGCTTTGCTGCTGAATTCAAGTTGGTCGACCAGTCCGGACCT
CCTCACGAGCCCAAGTTCGTTTACCAAGCAAAAGTTGGGGGTCGCTGGTT
CCCAGCCGTCTGCGCACACAGCAAGAAGCAAGGCAAGCAGGAAGCAGCAG
ATGCGGCTCTCCGTGTCTTGATTGGGGAGAACGAGAAGGCAGAACGCATG
GGTTTCACAGAGGTAACCCCAGTGACAGGGGCCAGTCTCAGAAGAACTAT
GCTCCTCCTCTCAAGGTCCCCAGAAGCACAGCCAAAGACACTCCCTCTCA
CTGGCAGCACCTTCCATGACCAGATAGCCATGCTGAGCCACCGGTGCTTC
AACACTCTGACTAACAGCTTCCAGCCCTCCTTGCTCGGCCGCAAGATTCT
GGCCGCCATCATTATGAAAAAAGACTCTGAGGACATGGGTGTCGTCGTCA
GCTTGGGAACAGGGAATCGCTGTGTGAAAGGAGATTCTCTCAGCCTAAAA
GGAGAAACTGTCAATGACTGCCATGCAGAAATAATCTCCCGGAGAGGCTT
CATCAGGTTTCTCTACAGTGAGTTAATGAAATACAACTCCCAGACTGCGA
AGGATAGTATATTTGAACCTGCTAAGGGAGGAGAAAAGCTCCAAATAAAA
AAGACTGTGTCATTCCATCTGTATATCAGCACTGCTCCGTGTGGAGATGG
CGCCCTCTTTGACAAGTCCTGCAGCGACCGTGCTATGGAAAGCACAGAAT
CCCGCCACTACCCTGTCTTCGAGAATCCCAAACAAGGAAAGCTCCGCACC
AAGGTGGAGAACGGAGAAGGCACAATCCCTGTGGAATCCAGTGACATTGT
GCCTACGTGGGATGGCATTCGGCTCGGGGAGAGACTCCGTACCATGTCCT
GTAGTGACAAAATCCTACGCTGGAACGTGCTGGGCCTGCAAGGGGCACTG
TTGACCCACTTCCTGCAGCCCATTTATCTCAAATCTGTCACATTGGGTTA
CCTTTTCAGCCAAGGGCATCTGACCCGTGCTATTTGCTGTCGTGTGACAA
GAGATGGGAGTGCATTTGAGGATGGACTACGACATCCCTTTATTGTCAAC
CACCCCAAGGTTGGCAGAGTCAGCATATATGATTCCAAAAGGCAATCCGG
GAAGACTAAGGAGACAAGCGTCAACTGGTGTCTGGCTGATGGCTATGACC
TGGAGATCCTGGACGGTACCAGAGGCACTGTGGATGGGCCACGGAATGAA
TTGTCCCGGGTCTCCAAAAAGAACATTTTTCTTCTATTTAAGAAGCTCTG
CTCCTTCCGTTACCGCAGGGATCTACTGAGACTCTCCTATGGTGAGGCCA
AGAAAGCTGCCCGTGACTACGAGACGGCCAAGAACTACTTCAAAAAAGGC
CTGAAGGATATGGGCTATGGGAACTGGATTAGCAAACCCCAGGAGGAAAA
GAACTTTTATCTCTGCCCAGTATAGGCTAGCTCAGATCAGGCCGGCCCTA
TCTGCACGTGCGTACAGCGATCGCCAAATTCGATATCAAGCTTATCGATA
ATCAACCTCTGGATTACAAAATTTGTGAAAGATTGACTGGTATTCTTAAC
TATGTTGCTCCTTTTACGCTATGTGGATACGCTGCTTTAATGCCTTTGTA
TCATGCTATTGCTTCCCGTATGGCTTTCATTTTCTCCTCCTTGTATAAAT
CCTGGTTGCTGTCTCTTTATGAGGAGTTGTGGCCCGTTGTCAGGCAACGT
GGCGTGGTGTGCACTGTGTTTGCTGACGCAACCCCCACTGGTTGGGGCAT
TGCCACCACCTGTCAGCTCCTTTCCGGGACTTTCGCTTTCCCCCTCCCTA
TTGCCACGGCGGAACTCATCGCCGCCTGCCTTGCCCGCTGCTGGACAGGG
GCTCGGCTGTTGGGCACTGACAATTCCGTGGTGTTGTCGGGGAAATCATC
GTCCTTTCCTTGGCTGCTCGCCTGTGTTGCCACCTGGATTCTGCGCGGGA
CGTCCTTCTGCTACGTCCCTTCGGCCCTCAATCCAGCGGACCTTCCTTCC
CGCGGCCTGCTGCCGGCTCTGCGGCCTCTTCCGCGTCTTCGCCTTCGCCC
TCAGACGAGTCGGATCTCCCTTTGGGCCGCCTCCCCGCATCGATACCGTC
GACCTCGACCTCGACTGTGCCTTCTAGTTGCCAGCCATCTGTTGTTTGCC
CCTCCCCCGTGCCTTCCTTGACCCTGGAAGGTGCCACTCCCACTGTCCTT
TCCTAATAAAATGAGGAAATTGCATCGCATTGTCTGAGTAGGTGTCATTC
TATTCTGGGGGGTGGGGTGGGGCAGGACAGCAAGGGGGAGGATTGGGAAG
ACAATGGCAGGCATGCTGGGGAACTAGTACGCGTCGGAGGGGGGAGGGGA
GTGTTGCAATACCTTTCTGGGAGTTCTCTGCTGCCTCCTGGCTTCTGAGG
ACCGCCCTGGGCCTGGGAGAATCCCTTCCCCCTCTTCCCTCGTGATCTGC
AACTCCAGTCTTTCTAGAAGATGGGCGGGAGTCTTCTGGGCAGGCTTAAA
GGCTAACCTGGTGTGTGGGCGTTGTCCTGCAGGGGAATTGAACAGGTGTA
AAATTGGAGGGACAAGACTTCCCACAGATTTTCGGTTTTGTCGGGAAGTT
TTTTAATAGGGGCAAATAAGGAAAATGGGAGGATAGGTAGTCATCTGGGG
TTTTATGCAGCAAAACTACAGGTTATTATTGCTTGTGATCCGCCTCGGAG
TATTTTCCATCGAGGTAGATTAAAGACATGCTCACCCGAGTTTTATACTC
TCCTGCTTGAGATCCTTACTACAGTATGAAATTACAGTGTCGCGAGTTAG
ACTATGTAAGCAGAATTTTAATCATTTTTAAAGAGCCCAGTACTTCATAT
CCATTTCTCCCGCTCCTTCTGCAGCCTTATCAAAAGGTATTTTAGAACAC
TCATTTTAGCCCCATTTTCATTTATTATACTGGCTTATCCAACCCCTAGA
CAGAGCATTGGCATTTTCCCTTTCCTGATCTTAGAAGTCTGATGACTCAT
GAAACCAGACAGATTAGTTACATACACCACAAATCGAGGCTGTAGCTGGG
GCCTCAACACTGCAGTTCTTTTATAACTCCTTAGTACACTTTTTGTTGAT
CCTTTGCCTTGATCCTTAATTTTCAGTGTCTATCACCTCTCCCGTCAGGT
GGTGTTCCACATTTGGGCCTATTCTCAGTCCAGGGAGTTTTACAACAATA
GATGTATTGAGAATCCAACCTAAAGCTTAACTTTCCACTCCCATGAATGC
CTCTCTCCTTTTTCTCCATTTATAAACTGAGCTATTAACCATTAATGGTT
TCCAGGTGGATGTCTCCTCCCCCAATATTACCTGATGTATCTTACATATT
GCCAGGCTGATATTTTAAGACATTAAAAGGTATATTTCATTATTGAGCCA
CATGGTATTGATTACTGCTTACTAAAATTTTGTCATTGTACACATCTGTA
AAAGGTGGTTCCTTTTGGAATGCAAAGTTCAGGTGTTTGTTGTCTTTCCT
GACCTAAGGTCTTGTGAGCTTGTATTTTTTCTATTTAAGCAGTGCTTTCT
CTTGGACTGGCTTGACTCATGGCATTCTACACGTTATTGCTGGTCTAAAT
GTGATTTTGCCAAGCTTCTTCAGGACCTATAATTTTGCTTGACTTGTAGC
CAAACACAAGTAAAATGATTAAGCAACAAATGTATTTGTGAAGCTTGGTT
TTTAGGTTGTTGTGTTGTGTGTGCTTGTGCTCTATAATAATACTATCCAG
GGGCTGGAGAGGTGGCTCGGAGTTCAAGAGCACAGACTGCTCTTCCAGAA
GTCCTGAGTTCAATTCCCAGCAACCACATGGTGGCTCACAACCATCTGTA
ATGGGATCTGATGCCCTCTTCTGGTGTGTCTGAAGACCACAAGTGTATTC
ACATTAAATAAATAAATCCTCCTTCTTCTTCTTTTTTTTTTTTTTAAAGA
GAATACTGTCTCCAGTAGAATTTACTGAAGTAATGAAATACTTTGTGTTT
GTTCCAATATGGTAGCCAATAATCAAATTACTCTTTAAGCACTGGAAATG
TTACCAAGGAACTAATTTTTATTTGAAGTGTAACTGTGGACAGAGGAGCC
ATAACTGCAGACT

### Nuclease Agents Facilitating Targeting Vector Integration

In some embodiments, nuclease agents (e.g., CRISPR/Cas systems, Zinc Finger Nucleases, and/or TALENs) can be employed in combination with targeting vectors to facilitate the modification of a target locus (e.g., modification of an ADAR1 locus and/or modification of a locus targeted for exogenous protein insertion). Such nuclease agents and their use are well known in the art, and may promote homologous recombination between a targeting vector and a target locus. When nuclease agents are employed in combination with a targeting vector, the targeting vector can comprise 5′ and 3′ homology arms corresponding to 5′ and 3′ target sequences located in sufficient proximity to a nuclease cleavage site so as to promote the occurrence of a homologous recombination event between target sequences and homology arms upon a nick or double-strand break at the nuclease cleavage site. In some embodiments, the term “nuclease cleavage site” includes a DNA sequence at which a nick or double-strand break is created by a nuclease agent (e.g., a Cas9 cleavage site). Target sequences within a targeted locus that correspond to 5′ and 3′ homology arms of a targeting vector are “located in sufficient proximity” to a nuclease cleavage site if the distance is such as to promote the occurrence of a homologous recombination event between 5′ and 3′ target sequences and homology arms upon a nick or double-strand break at the recognition site. Thus, in certain embodiments, target sequences corresponding to 5′ and/or 3′ homology arms of a targeting vector are within at least one nucleotide of a given recognition site or are within at least 10 nucleotides to about 14 kb of a given recognition site. In some embodiments, a nuclease cleavage site is immediately adjacent to at least one, two, three, four, and/or more target sequences.

The spatial relationship of target sequences that correspond to homology arms of a targeting vector and a nuclease cleavage site can vary. For example, target sequences can be located 5′ to a nuclease cleavage site, target sequences can be located 3′ to a recognition site, or target sequences can flank a nuclease cleavage site.

Combined use of a targeting vector with a nuclease agent can result in an increased targeting efficiency compared to use of a targeting vector alone. For example, when a targeting vector is used in conjunction with a nuclease agent, targeting efficiency of a targeting vector can be increased by at least two-fold, at least three-fold, at least four-fold, at least five-fold, at least six-fold, at least seven-fold, at least eight-fold, at least nine-fold, at least ten-fold or within a range formed from these integers, such as 2-10-fold when compared to use of a targeting vector alone.

In some embodiments, targeting vectors comprise homology arms that correspond to and are derived from nucleic acid sequences larger than those typically used by other approaches intended to perform homologous recombination in cells. In some embodiments, targeting vectors comprise homology arms that correspond to and are derived from nucleic acid sequences shorter than those typically used by other approaches intended to perform homologous recombination in cells. In some embodiments, a homology arm is at least 10 kb in length, or the sum total of a 5′ homology arm and a 3′ homology arm can be, for example, at least 10 kb. In some embodiments, a homology arm is less than 10 kb in length, or the sum total of a 5′ homology arm and a 3′ homology arm can be, for example, is less than10 kb.

In some embodiments, targeting vectors comprising nucleic acid constructs larger than those typically used by other approaches intended to perform homologous recombination in cells. For example, in some embodiments, large loci that cannot traditionally be accommodated by plasmid-based targeting vectors because of their size limitations may still be employed through the use of large targeting vectors. For example, a targeted locus can be (i.e., 5′ and 3′ homology arms can correspond to) a locus of a cell that is not targetable using a conventional method or that can be targeted only incorrectly or only with significantly low efficiency in the absence of a nick or double-strand break induced by a nuclease agent (e.g., a Cas protein). In some embodiments, a large targeting vector may include vectors derived from bacterial artificial chromosome (BAC), a human artificial chromosome, or a yeast artificial chromosome (YAC). Large targeting vectors can be in linear form or in circular form. Examples of large targeting vectors and methods for making them are described, e.g., in Macdonald (2014), U.S. Pat. Nos. 6,586,251, 6,596,541 and No. 7,105,348; and International Patent Application Publication No. WO 2002/036789.

### Methods of Making Provided Non-Human Animals

Those skilled in the art appreciate that various technologies can be utilized to make engineered cells, issues, animals, etc. in accordance with the present disclosure. Provided herein are compositions and methods for making non-human animals (e.g., rodents, e.g., mice) whose germline genome comprises an engineered human ADAR1 gene that includes one or more functional ADAR1 domains (e.g., Z-binding domains, double-stranded RNA binding motifs, and/or RNA deaminase motifs). In some embodiments, methods described herein comprise inserting transcriptionally independent portions of a human ADAR protein which may be rejoined in vivo through the action of trans splice acceptors and/or donors.

In some embodiments, the non-human ADAR1 locus may be the site for insertion of a human ADAR1 gene. In some embodiments, any suitable integration locus may be the site for insertion of a human ADAR1 coding sequence.

In some embodiments, a human ADAR1 gene may be under the control of a heterologous protein enhancer(s) and/or promoter(s). In some embodiments, methods described herein comprise inserting a single human ADAR1 gene encoding a human ADAR protein. In some embodiments, methods described herein comprise inserting more than one human ADAR1 gene encoding more than one human ADAR polypeptides.

Provided herein are compositions and methods for making non-human animals (e.g., rodents, e.g., mice) whose germline genome comprises an engineered non-human primate (NHP) ADAR1 locus that includes one or more functional ADAR1 domains (e.g., Z-binding domains, double-stranded RNA binding motifs, and/or RNA deaminase motifs). In some embodiments, methods described herein comprise inserting transcriptionally independent portions of a NHP ADAR protein which may be rejoined in vivo through the action of trans splice acceptors and/or donors.

In some embodiments, the non-human ADAR1 locus may be the site for insertion of a NHP ADAR1 gene. In some embodiments, any suitable integration locus may be the site for insertion of a NHP ADAR1 coding sequence.

In some embodiments, a NHP ADAR1 gene may be under the control of a heterologous protein enhancer(s) and/or promoter(s). In some embodiments, methods described herein comprise inserting a single NHP ADAR1 gene encoding a NHP ADAR protein. In some embodiments, methods described herein comprise inserting more than one NHP ADAR1 gene encoding a NHP ADAR polypeptide.

### Embryonic Stem Cell Modification for Creating Transgenic Animals

In some embodiments, methods of making a provided non-human animal include insertion of genetic material that comprises an exogenous ADAR1 gene into an embryonic stem cell of a non-human animal (e.g., a rodent, e.g., a rat or mouse). In some embodiments, methods include multiple insertions in a single ES cell clone. In some embodiments, methods include sequential insertions made in a successive ES cell clones. In some embodiments, methods include a single insertion made in an engineered ES cell clone.

In some embodiments, methods of making a non-human transgenic animal involving the use of an embryonic stem cell can have a targeting vector and/or nucleic acid construct introduced through any manner known in the art. In some embodiments, a transgene is introduced to an embryonic stem cell through a method comprising but not limited to: electroporation, lipid based transfection, lipid based nanoparticles, retroviral infection, and/or lentiviral infection.

In some embodiments of a method of making a non-human animal (e.g., rodent, e.g., mouse), a DNA fragment is introduced into a non-human embryonic stem cell.

In some embodiments, methods comprising the use of embryonic stem cell modification for the creation of transgenic animals may utilize any molecular biology technique or reagent described herein.

In some embodiments, a targeting vector comprising an ADAR1 coding sequence is electroporated into mouse ES cells, using methods known in the art. Screening and/or selection for clones that have undergone homologous recombination yields modified ES cells for generating chimeric mice that express huADAR1. Positive ES cell clones are confirmed by PCR screening using primers and probes specific for the huADAR1 transgene. Primers and probes vary dependent upon the insertion loci of interest. Targeted ES cells are used as donor ES cells and introduced into an 8-cell stage mouse embryo using an appropriate method (e.g., by the VELOCIMOUSE® method (see, e.g., U.S. Pat. No. 7,294,754 and Poueymirou et al. (2007). F0 generation mice that are essentially fully derived from the donor gene-targeted ES cells allowing immediate phenotypic analyses, Nature Biotech. 25(1): 91-99). Transgenic mice expressing huADAR1 are identified by genotyping using methods known in the art. Mice are bred to stable heterozygotic and/or homozygotic transgenic transmission of a huADAR1 insertion locus.

### Zygotic Infection for Creating Transgenic Animals

Where appropriate, an exogenous ADAR1 gene (e.g., a human ADAR1 encoding a human ADAR1 protein) may separately be modified to include codons that are optimized for expression in a non-human animal (e.g., see U.S. Pat. Nos. 5,670,356 and 5,874,304). Codon optimized sequences are engineered sequences, and preferably encode the identical polypeptide (or a biologically active fragment of a characteristic portion of the polypeptide which has substantially the same activity as the full-length polypeptide) encoded by the non-codon optimized parent polynucleotide. In some embodiments, an exogenous ADAR1 gene encoding an exogenous ADAR1 protein may separately include an altered sequence to optimize codon usage for a particular cell type (e.g., a rodent cell, e.g., a mouse cell). For example, the codons of each nucleotide sequence to be inserted into the genome of a non-human animal as described herein (e.g., a rodent, e.g., mouse) may be optimized for expression in a cell of the non-human animal. Such a sequence may be described as a codon-optimized sequence.

In some embodiments, insertion of nucleotide sequences encoding an exogenous ADAR1 gene employs a minimal amount of modification of the germline genome of a non-human animal as described herein and results in expression of an exogenous ADAR1 gene (e.g., a human ADAR1 gene or a NHP ADAR1 gene). Methods for generating engineered non-human animals (e.g., rodents, e.g., rats or mice), including knockouts and knock-ins, are known in the art (see, e.g., Gene Targeting: A Practical Approach, Joyner, ed., Oxford University Press, Inc., 2000). For example, generation of genetically engineered rodents may optionally involve disruption of the genetic loci of one or more endogenous rodent genes (or gene segments) and introduction of one or more heterologous genes (or gene segments or nucleotide sequences) into the rodent genome, in some embodiments, at the same location as an endogenous rodent gene (or gene segments). In some embodiments, nucleotide sequences encoding an exogenous ADAR1 gene (e.g., a human ADAR1 gene or a NHP ADAR1 gene) is randomly inserted in the germline genome of a rodent. In some embodiments, nucleotide sequences encoding an exogenous ADAR1 gene are introduced upstream of a non-human (e.g., rodent, e.g., rat or mouse) ADAR1 locus in the germline genome of a rodent; in some certain embodiments, an endogenous ADAR1 locus is altered, modified, or engineered to contain human and/or NHP ADAR1 gene segments, wherein any combination of ADAR1 gene segments derived from rodent, human, and/or NHP may be utilized.

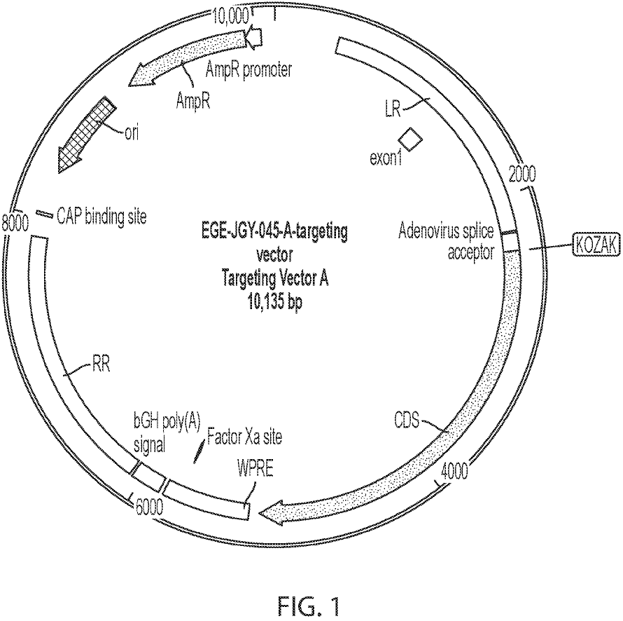

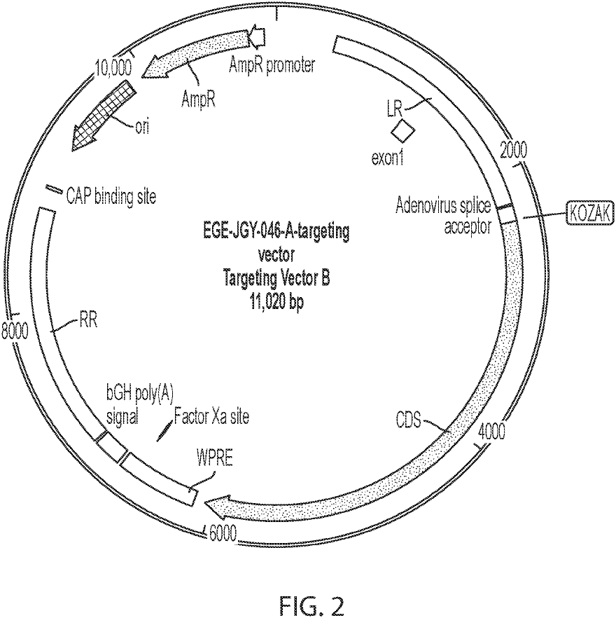

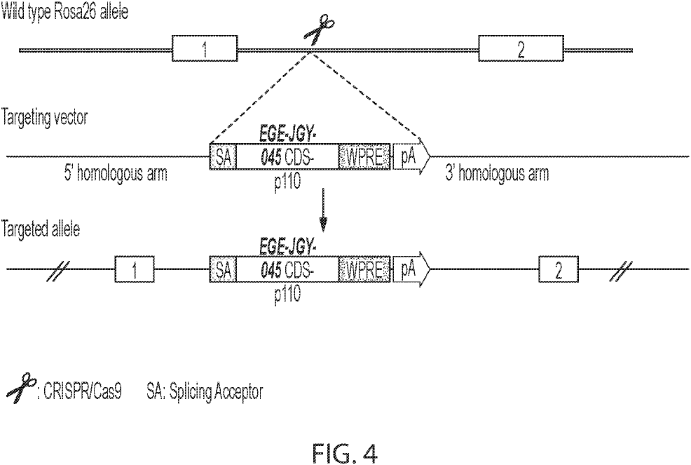

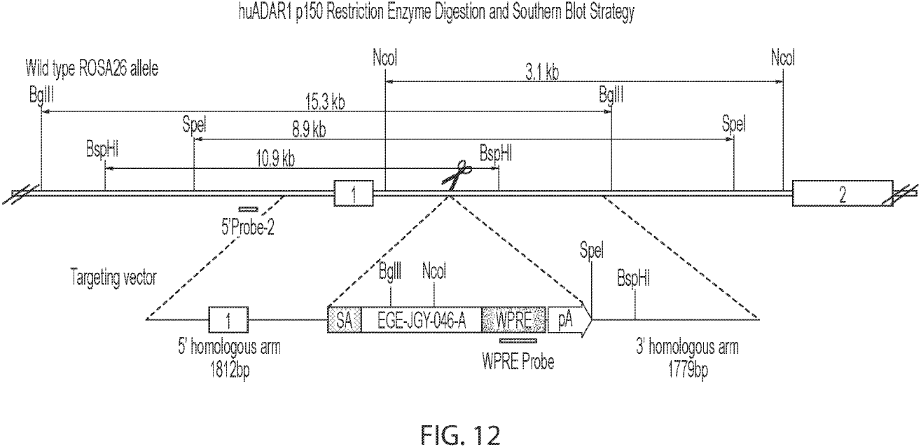

Schematic illustrations (not to scale) of exemplary nucleic acid constructs engineered to introduce an exogenous ADAR1 gene into the mouse germline genome are provided in FIGS. 1 and 2. The predicted integration of these constructs into the mouse ROSA26 locus is illustrated in FIGS. 4 and 12. Methods for constructing such an engineered nucleic acid construct comprising a targeting vector are known in the art. Once produced, a targeting vector can be linearized injected into a rodent zygote or alternatively electroporated into rodent embryonic stem (ES) cells to create a rodent whose germline genome comprises the exogenous ADAR1 gene. In some embodiments, confirmation of rodent ES cells comprising a targeting vector comprising an exogenous ADAR1 gene can be selected and/or screened for using methods known in the art. As described in the examples section below, rodent zygotes comprising an injected nucleic acid construct comprising a targeting vector comprising an exogenous ADAR1 gene can be utilized for creating transgenic non-human animals comprising an integrated exogenous ADAR1 gene, such animals can be screened for from a population of viable injected zygotes which have been transplanted into a surrogate mother.

In some embodiments, a targeting vector is introduced into non-human (e.g., rodent, e.g., mouse or rat) embryonic cells (e.g., zygotes and/or stem cells) by electroporation so that the sequence contained in the targeting vector results in the capacity of a non-human (e.g., rodent, e.g., rat or mouse) cell or non-human animal (e.g., a rodent, e.g., rat or mouse) to expresses an exogenous ADAR1 gene. As described herein, a genetically engineered non-human animal is generated where an exogenous ADAR1 gene has been created and/or incorporated into the germline genome of the non-human animal (e.g., at a defined locus, and/or at a random locus). In some embodiments, insertion and/or expression of an exogenous ADAR1 gene is confirmed using methods known in the art (e.g., PCR, western blotting etc.) In some embodiments, oligonucleotides as described herein are then characterized in vitro or in vivo using tissues, cells, and/or animals derived from a non-human embryonic stem cell comprising an exogenous ADAR1 gene.

In some embodiments, a method of making a genetically modified non-human animal (e.g., rodent, e.g., mouse) comprises engineering a human ADAR1 gene in the germline genome of the non-human animal to comprise a sequence operably linked to a tissue specific regulatory region.

In some embodiments, a method of making a genetically modified non-human animal (e.g., rodent, e.g., mouse) comprises engineering a human ADAR1 gene in the germline genome of the non-human animal to comprise a sequence operably linked to a temporally specific regulatory region.

In some embodiments, a method of making a genetically modified non-human animal (e.g., rodent, e.g., mouse) comprises engineering a human ADAR1 gene in the germline genome of the non-human animal to comprise a sequence operably linked to a substrate specific regulatory region.

In some embodiments, a non-human animal (e.g., rodent, e.g., rat or mouse) made, generated, produced, obtained or obtainable from a method as described herein is provided.

In some embodiments of a method of making a non-human animal (e.g., rodent, e.g., rat or mouse), a DNA fragment is introduced into a non-human embryonic stem cell and/or zygote whose germline genome comprises an endogenous ADAR1 loci. Alternatively, and/or additionally, in some embodiments, the germline genome of a non-human animal (e.g., rodent, e.g., rat or mouse) as described herein further comprises a deleted, inactivated, functionally silenced or otherwise non-functional endogenous ADAR1 locus. Genetic modifications to delete or render non-functional a gene or genetic locus may be achieved using methods described herein and/or methods known in the art.

A genetically engineered founder non-human animal (e.g., rodent, e.g., rat or mouse) can be identified based upon the presence of an exogenous ADAR1 gene as described herein in its germline genome and/or expression of exogenous ADAR1 protein in tissues or cells of the non-human animal. A genetically engineered founder non-human animal can then be used to breed additional non-human animals carrying an exogenous ADAR1 gene, thereby creating a cohort of non-human animals each carrying one or more copies of an exogenous ADAR1 gene. Moreover, genetically engineered non-human animals carrying an exogenous ADAR1 gene can further be bred to other genetically engineered non-human animals carrying other transgenes (e.g., human disease genes) or other mutated endogenous loci as desired.

Genetically engineered non-human animals (e.g., rodents, e.g., rats or mice) may also be produced to contain selected systems that allow for regulated, directed, inducible and/or cell-type specific expression of a transgene or integrated sequence(s). For example, non-human animals as described herein may be engineered to contain one or more sequences encoding an exogenous ADAR1 gene that is/are conditionally expressed (e.g., reviewed in Rajewski, K. et al., 1996, J. Clin. Invest. 98(3):600-3). Exemplary systems include the Cre/loxP recombinase system of bacteriophage P1 (see, e.g., Lakso, M. et al., 1992, Proc. Natl. Acad. Sci. U.S.A. 89:6232-6) and the FLP/Frt recombinase system of S. cerevisiae (O′Gorman, S. et al, 1991, Science 251:1351-5). Such animals can be provided through the construction of “double” genetically engineered animals, e.g., by mating two genetically engineered animals, one containing a transgene comprising a selected modification (e.g., an exogenous ADAR1 gene as described herein) and the other containing a transgene encoding a recombinase (e.g., a Cre recombinase).

### Non-Human Animals Amenable to Exogenous ADAR1 Gene Expression

Non-human animals (e.g., rodents, e.g., rats or mice) as described herein may be prepared as described above, or using methods known in the art, to comprise additional human, humanized or otherwise engineered genes, oftentimes depending on the intended use of the non-human animal. Genetic material of such human, humanized or otherwise engineered genes may be introduced through the further alteration of the genome of cells (e.g., embryonic stem cells, and/or injection of zygotes derived from transgenic rodents comprising an exogenous ADAR1 gene) having the genetic modifications or alterations as described above or through breeding techniques known in the art with other genetically modified or engineered strains as desired.

As those skilled in the art appreciate, various compatible mouse strains (e.g., WT, harboring one or more transgenes, containing one or more mutations in an endogenous loci, etc.), can be bred to any one of the engineered mice described herein to create any number of genetically modified mouse strains expressing an ADAR1 (e.g., a NHP ADAR1, a human ADAR1, etc.) polypeptide or a characteristic portion thereof and any additional genetic features (e.g., natural mouse mutant loci, disease modelling endogenous mouse gene mutant loci, transgenically derived mutant animals expressing a human gene mutation of interest, etc.). Various technologies can be utilized to generate mice heterozygous or homozygous for a transgenic polynucleotide encoding an ADAR1 polypeptide or a characteristic portion thereof as described herein (e.g., human ADAR1). In some embodiments, genetically modified mice which are homozygous or heterozygous for huADAR1 (e.g., those described in the Examples) are bred to mice homozygous or heterozygous for a mutation (deletion, gain of function, loss of function, etc.) of an endogenous mouse gene of interest that may be associated with ADAR function. Resultant progeny expressing desired ADAR1 or characteristic portions thereof and heterozygous for a gene of interest are crossed to obtain mice homozygous and/or heterozygous for ADAR1 and/or the gene of interest. In some embodiments, breeding may be performed by a commercial breeder (e.g., The Jackson Laboratory). In certain embodiments, mice heterozygous for a transgenic ADAR1 insertion (e.g., as described herein) are crossed to a balancer line to maintain stable heterozygotic transgenic ADAR1 transmission. In some embodiments, a closely linked phenotypically detectable marker is genetically engineered into transgenic ADAR1 mice to aid with crossing and/or genotyping.

Although embodiments describing the construction of an exogenous ADAR1 gene in a mouse (i.e., a mouse with an exogenous ADAR1 gene integrated into its germline genome) are extensively discussed herein, other non-human animals that comprise an exogenous ADAR1 gene are also provided. Such non-human animals include any of those which can be genetically modified to express exogenous ADAR1 polypeptides and/or fragments thereof as described herein, including, e.g., mammals, e.g., mouse, rat, rabbit, pig, bovine (e.g., cow, bull, buffalo), deer, sheep, goat, chicken, cat, dog, ferret, primate (e.g., marmoset, rhesus monkey), etc. For example, for those non-human animals for which suitable genetically modifiable ES cells are not readily available, other methods are employed to make a non-human animal comprising the genetic modification. Such methods include, e.g., modifying a non-ES cell genome (e.g., a fibroblast or an induced pluripotent cell) and employing somatic cell nuclear transfer (SCNT) to transfer the genetically modified genome to a suitable cell, e.g., an enucleated oocyte, and gestating the modified cell (e.g., the modified oocyte) in a non-human animal under suitable conditions to form an embryo.

Methods for modifying the germline genome of a non-human animal (e.g., a pig, cow, rodent, chicken, etc. genome) include, e.g., employing a zinc finger nuclease (ZFN), a transcription activator-like effector nuclease (TALEN), or a Cas protein (i.e., a CRISPR/Cas system) to include an exogenous ADAR1 gene. Guidance for methods for modifying the germline genome of a non-human animal can be found in, e.g., U.S. Pat. No. 9,738,897, and U.S. Pat. Application Publication Nos. US 2016/0145646 (published May 26, 2016) and US 2016/0177339 (published Jun. 23, 2016).

In some embodiments, a non-human animal as described herein is a mammal. In some embodiments, a non-human animal as described herein is a small mammal, e.g., of the superfamily Dipodoidea or Muroidea. In some embodiments, a genetically modified animal as described herein is a rodent. In some embodiments, a rodent as described herein is selected from a mouse, a rat, and a hamster. In some embodiments, a rodent as described herein is selected from the superfamily Muroidea. In some embodiments, a genetically modified animal as described herein is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, white-tailed rats, Malagasy rats and mice), Platacanthomyidae (e.g., spiny dormice), and Spalacidae (e.g., mole rates, bamboo rats, and zokors). In some certain embodiments, a genetically modified rodent as described herein is selected from a true mouse or rat (family Muridae), a gerbil, a spiny mouse, and a crested rat. In some certain embodiments, a genetically modified mouse as described herein is from a member of the family Muridae. In some embodiment, a non-human animal as described herein is a rodent. In some certain embodiments, a rodent as described herein is selected from a mouse and a rat. In some embodiments, a non-human animal as described herein is a mouse.

In some embodiments, a non-human animal as described herein is a rodent that is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some certain embodiments, a mouse as described herein is a 129-strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129/SvJae, 129S6 (129/SvEvTac), 129S7, 129S8, 129T1, 129T2 (see, e.g., Festing et al., 1999, Mammalian Genome 10:836; Auerbach, W. et al., 2000, Biotechniques 29(5):1024-1028, 1030, 1032). In some certain embodiments, a genetically modified mouse as described herein is a mix of an aforementioned 129 strain and an aforementioned C57BL/6 strain. In some certain embodiments, a mouse as described herein is a mix of aforementioned 129 strains, or a mix of aforementioned BL/6 strains. In some certain embodiments, a 129 strain of the mix as described herein is a 129S6 (129/SvEvTac) strain. In some embodiments, a mouse as described herein is a BALB strain, e.g., BALB/c strain. In some embodiments, a mouse as described herein is a mix of a BALB strain and another aforementioned strain.

In some embodiments, a non-human animal as described herein is a rat. In some certain embodiments, a rat as described herein is selected from a Wistar rat, an LEA strain, a Sprague Dawley strain, a Fischer strain, F344, F6, and Dark Agouti. In some certain embodiments, a rat strain as described herein is a mix of two or more strains selected from the group consisting of Wistar, LEA, Sprague Dawley, Fischer, F344, F6, and Dark Agouti.

A rat pluripotent and/or totipotent cell can be from any rat strain, including, for example, an ACI rat strain (an inbred strain originally derived from August and Copenhagen strains), a Dark Agouti (DA) rat strain, a Wistar rat strain, a LEA rat strain, a Sprague Dawley (SD) rat strain, or a Fischer rat strain such as Fisher F344 or Fisher F6. Rat pluripotent and/or totipotent cells can also be obtained from a strain derived from a mix of two or more strains recited above. For example, the rat pluripotent and/or totipotent cell can be from a DA strain or an ACI strain. The ACI rat strain is characterized as having black agouti, with white belly and feet and an RT1av1 haplotype. Such strains are available from a variety of sources including Harlan Laboratories. An example of a rat ES cell line from an ACI rat is an ACI.G1 rat ES cell. The DA rat strain is characterized as having an agouti coat and an RT1av1 haplotype. Such rats are available from a variety of sources including Charles River and Harlan Laboratories. Examples of a rat ES cell line from a DA rat are the DA.2B rat ES cell line and the DA.2C rat ES cell line. In some embodiments, the rat pluripotent and/or totipotent cells are from an inbred rat strain (see, e.g., U.S. Pat. Application Publication No. 2014-0235933 A1). Guidance for making modifications in a rat genome (e.g., in a rat ES cell) using methods and/or constructs as described herein can be found in, e.g., in U.S. Pat. Application Publication Nos. 2014-0310828 and 2017-0204430.

In some embodiments, useful technologies are described in, e.g., US10314297 and can be utilized in accordance with the present disclosure. As those skilled in the art appreciate, many useful technologies are commercially available from various venders and/or service providers.

In some embodiments, a non-human animal (e.g., mouse) comprising or expressing ADAR1 polypeptide or a characteristic portion thereof is bred with a second non-human animal (e.g., mouse) which comprises an adenosine to be edited. In some embodiments, a second non-human animal is an animal model for a condition, disorder or disease, for example, one that may benefit from adenosine editing (e.g., one associated with G to A mutations). Those skilled in the art appreciate that adenosine editing may provide benefits in a variety of potential mechanisms, such as one or more of splicing modulation (increasing or decreasing levels/activities of one or more transcripts and/or products encoded thereby), reduction of levels/activities of one or more transcripts and/or products encoded thereby (e.g., through introducing A to I mutations), increase of levels/activities of one or more transcripts and/or products encoded thereby (e.g., through correction of G to A mutations), etc. In some embodiments, a breeding product is a non-human animal comprising or expressing ADAR1 polypeptide or a characteristic portion thereof as described herein, and comprising a target adenosine (e.g., an adenosine associated with a condition, disorder or disease which can benefit from adenosine editing). In some embodiments, such non-human animals and cells and/or tissues therefrom are useful for assessing various agents, e.g., oligonucleotides, and compositions for identifying, assessing, developing, etc., agents and compositions useful for editing target adenosines for, e.g., various biological and/or therapeutic applications (e.g., for preventing and/or treating a condition, disorder or disease that may benefit from an adenosine editing).

For example, in some embodiments, a second animal is a useful model for alpha 1-antitrypsin (A1AT) deficiency. In some embodiments, a second animal comprises a G to A mutation that corresponds to 1024 G>A (E342K) mutation in human SERPINA1 gene. In some embodiments, a second animal is humanized and comprises a human SERPINA1 gene or a fragment thereof. In some embodiments, a fragment comprises one or more mutations associated with one or more conditions, disorders or diseases. In some embodiments, a mutation is 1024 G>A (E342K). In some embodiments, a second animal is humanized and comprises a human SERPINA1 gene comprising a Pi*Z mutant allele. In some embodiments, a second animal is a NOD.Cg-Prkdcscid Il2rgtm1Wjl Tg(SERPINA1*E342K)#Slcw/SzJ mouse (e.g., see The Jackson Laboratory Stock No: 028842; NSG-PiZ, and also Borel F; Tang Q; Gernoux G; Greer C; Wang Z; Barzel A; Kay MA; Shultz LD; Greiner DL; Flotte TR; Brehm MA; Mueller C. 2017. Survival Advantage of Both Human Hepatocyte Xenografts and Genome-Edited Hepatocytes for Treatment of alpha-1 Antitrypsin Deficiency. Mol Ther 25(11):2477-2489PubMed: 29032169MGI: J:243726, and Li S; Ling C; Zhong L; Li M; Su Q; He R; Tang Q; Greiner DL; Shultz LD; Brehm MA; Flotte TR; Mueller C; Srivastava A; Gao G. 2015. Efficient and Targeted Transduction of Nonhuman Primate Liver With Systemically Delivered Optimized AAV3B Vectors. Mol Ther 23(12):1867-76PubMed: 26403887MGI: J:230567). In some embodiments, a product cell, tissue, or non-human animal comprises a G to A mutation that corresponds to 1024 G>A (E342K) mutation in human SERPINA1 gene, and comprises or expresses an ADAR1 polypeptide or a characteristic portion thereof as described herein. In some embodiments, a product cell, tissue, or non-human animal comprises a human SERPINA1 Pi*Z allele comprising a G to A mutation that corresponds to 1024 G>A (E342K) mutation, and comprises or expresses a human ADAR1 polypeptide or a characteristic portion thereof as described herein. In some embodiments, a product cell, tissue, or non-human animal comprises a human SERPINA1 Pi*Z allele comprising a G to A mutation that corresponds to 1024 G>A (E342K) mutation, and is wild type and/or does not expresses a human ADAR1 polypeptide or a characteristic portion thereof as described herein, and may act as a relative control.

Among other things, mutations in the SERPINA1 gene have been reported to cause alpha 1-antitrypsin (A1AT) deficiency. Mutations leading to A1AT deficiency can sometimes be described based on their target positions in a SERPINA1 gene that encode the mutated amino acids. In the United States, the prevalence of A1AT deficiency is reported to be between 1 in 5,000 and 1 in 7,000. A1AT deficiency is reported to be one of the most common genetic diseases in subjects of Northern European descent. In some embodiments, severe A1AT deficiency causes emphysema, with subjects developing emphysema in their third or fourth decade. It has been reported that A1AT deficiency can also cause liver failure and hepatocellular carcinoma, with up to 30% of subjects with severe A1AT deficiency developing significant liver disease, including cirrhosis, fulminant liver failure, and hepatocellular carcinoma.

It is reported that there are two predominant mutations in the SERPINA1 gene that cause A1AT deficiency. These missense mutations reportedly affect protein conformation and secretion leading to reduced circulating levels of A1AT. It is reported that the more common and more severe mutation is a glutamate to lysine substitution at amino acid position 342 (E342K, “Z mutation”) of the mature A1AT protein, which can arise from, e.g., c.1024G>A mutation. Alleles carrying the Z mutation are sometimes identified as PiZ alleles. Subjects homozygous for the PiZ allele are termed PiZZ carriers, and reportedly express 10-15% of normal levels of serum A1AT. It is reported that approximately 95% of subjects who are symptomatic for A1AT deficiency have the PiZZ genotype. In some embodiments, such a mutation is recapitulated in a non-human model organism. In some embodiments, such a non-human model organism is a mouse that comprises a human SERPINA1 Pi*Z allele comprising a G to A mutation that corresponds to 1024 G>A (E342K) mutation.

In some embodiments, an A1AT target position comprises or consists of one or more nucleotide mutations in the SERPINA1 gene, which results in expression of an A1AT mutant protein comprising an amino acid mutation at E342. In certain embodiments, an A1AT target position comprises or consists of a nucleotide mutation at position c .1024 in the SERPINA1 gene, which results in expression of an A1AT mutant protein comprising an amino acid mutation at E342. In certain embodiments, an A1AT target position comprises or consists of the nucleotide mutation c .1024G>A in the SERPINA1 gene, which results in the expression of an A1AT mutant protein comprising the amino acid mutation E342K.

In certain embodiments, a non-human model organism comprising a human SERPINA1 Pi*Z allele may also comprise additional genetic mutations and/or modifications that render the animal humanized. In some embodiments, a humanized animal is immunodeficient, and in some embodiments, is extremely immunodeficient. In some embodiments, such an animal may have the genotype NOD.Cg-Prkdc^{scid} Il2rgt^{m1Wjl}/SzJ. In some embodiments, such mice carry two mutations on the NOD/ShiLtJ genetic background; severe combined immune deficiency (scid) and a complete null allele of the IL2 receptor common gamma chain (IL2rg^{null}). In some embodiments, a scid mutation is in the DNA repair complex protein Prkdc and renders the mice B and T cell deficient. In some embodiments, a IL2rg^{null} mutation prevents cytokine signaling through multiple receptors, leading to a deficiency in functional NK cells. In some embodiments, severe immunodeficiency allows the mice to be humanized, e.g., through methods known in the art such as engraftment of human CD34+ hematopoietic stem cells (HSC), peripheral blood mononuclear cells (PBMC), patient derived xenografts (PDX), and/or adult stem cells and tissues.

In some embodiments, the present disclosure provides methods for assessing an agent, e.g., an oligonucleotide, or a composition thereof, comprising administering to an animal, cell or tissue described herein the agent or composition. In some embodiments, an agent or composition is assessed for preventing or treating a condition, disorder or disease. In some embodiments, animals, cells, tissues, e.g., as described in various embodiments herein, are animal models, or cells or tissues, for various conditions, disorders or diseases (e.g., comprising mutations associated with various conditions, disorders or diseases, and/or cells, tissues, organs, etc., associated with or of various conditions, disorders or diseases) that are engineered to comprise and/or express a polynucleotide whose sequence encodes an ADAR1 polypeptide or a characteristic portion thereof. In some embodiments, animals may be provided by breeding (e.g., IVF, natural breeding, etc.) an animal that are model animals for various conditions, disorders or diseases but are not engineered to comprise and/or express a polynucleotide whose sequence encodes an ADAR1 polypeptide or a characteristic portion thereof with animals that are engineered to comprise and/or express a polynucleotide whose sequence encodes an ADAR1 polypeptide or a characteristic portion thereof. In some embodiments, cells or tissues may be provided by introducing into cells or tissues a polynucleotide whose sequence encodes an ADAR1 polypeptide or a characteristic portion thereof. In some embodiments, the present disclosure provides a method for preventing or treating a condition, disorder or disease, comprising administering to a subject an effective amount of an agent or a compositions thereof, wherein the agent or composition is assessed in an animal provided herein (e.g., an animal engineered to comprise an ADAR1 polypeptide or a characteristic portion thereof, an animal engineered to comprise and/or express a polynucleotide whose sequence encodes an ADAR1 polypeptide or a characteristic portion thereof, a model animal for a condition, disorder or disease which is engineered to comprise an ADAR1 polypeptide or a characteristic portion thereof, a model animal for a condition, disorder or disease engineered to comprise and/or express a polynucleotide whose sequence encodes an ADAR1 polypeptide or a characteristic portion thereof). In some embodiments, the present disclosure provides a method for preventing or treating a condition, disorder or disease, comprising administering to a subject an effective amount of an agent or a compositions thereof, wherein the agent or composition is assessed in a cell or tissue provided herein. In some embodiments, an animal, cell or tissue comprises a SERPINA1 mutation (e.g., 1024 G>A (E342K)) and is engineered to comprise and/or express a polynucleotide whose sequence encodes an ADAR1 polypeptide or a characteristic portion thereof. In some embodiments, an animal is a non-human animal. In some embodiments, cells are non-human animal cells. In some embodiments, tissues are non-human animal tissues. In some embodiments, a non-human animal is a rodent. In some embodiments, a non-human animal is a mouse. In some embodiments, a non-human animal is a rat. In some embodiments, a non-human animal is a non-human primate.

As appreciated by those skilled in the art, in some embodiments, animals can be heterozygous with respect to one or more or all sequences. In some embodiments, animals are homozygous with respect to one or more or all sequences. In some embodiments, animals are hemizygous with respect to one or more or all engineered sequences. In some embodiments, animals are homozygous with respect to one or more sequences, and heterozygous with respect to one or more sequences. In some embodiments, animals are heterozygous with respect to a polynucleotide whose sequence encodes an ADAR1 polypeptide or a characteristic portion thereof. In some embodiments, animals are homozygous with respect to a polynucleotide whose sequence encodes an ADAR1 polypeptide or a characteristic portion thereof. In some embodiments, animals are homozygous wild-type with respect to a loci encoding a polynucleotide whose sequence encodes an ADAR1 polypeptide or a characteristic portion thereof (e.g., do not express an exogenous ADAR1 polypeptide or a characteristic portion thereof), and may act as a relative control. In some embodiments, certain animals are heterozygous with respect to one or more polynucleotide sequences associated with various condition, disorder or diseases, and are heterozygous with respect to a polynucleotide whose sequence encodes an ADAR1 polypeptide or a characteristic portion thereof. In some embodiments, certain animals are homozygous with respect to one or more polynucleotide sequences associated with various condition, disorder or diseases, and are heterozygous with respect to a polynucleotide whose sequence encodes an ADAR1 polypeptide or a characteristic portion thereof. In some embodiments, certain animals are heterozygous with respect to one or more polynucleotide sequences associated with various condition, disorder or diseases, and are homozygous with respect to a polynucleotide whose sequence encodes an ADAR1 polypeptide or a characteristic portion thereof. In some embodiments, certain animals are homozygous with respect to one or more polynucleotide sequences associated with various condition, disorder or diseases, and are homozygous with respect to a polynucleotide whose sequence encodes an ADAR1 polypeptide or a characteristic portion thereof. Cells or tissues may be similarly heterozygous, hemizygous and/or homozygous with respect to various sequences.

In some embodiments, the present disclosure provides methods comprising: 1) assessing an agent or a composition thereof, comprising contacting the agent or a composition thereof with a provided cell or tissue associated with or of a condition, disorder or disease, and 2) administering to a subject suffering from or susceptible to a condition, disorder or disease an effective amount of an agent or composition thereof. In some embodiments, the present disclosure provides methods comprising: 1) assessing an agent or a composition thereof, comprising administering the agent or a composition thereof to a provided animal which is an animal model of a condition, disorder or disease, and 2) administering to a subject suffering from or susceptible to a condition, disorder or disease an effective amount of an agent or composition thereof. In some embodiments, as described herein, a cell, tissue or animal is engineered to comprise an ADAR1 polypeptide or a characteristic portion thereof. In some embodiments, a cell, tissue or animal is engineered to comprise and/or express a polynucleotide whose sequence encodes an ADAR1 polypeptide or a characteristic portion thereof. In some embodiments, a cell, tissue or animal further comprises a nucleotide sequence (e.g., a mutation) associated with a condition, disorder or disease. In some embodiments, an animal is a rodent, e.g., a mouse, a rat, etc. In some embodiments, a cell or tissue is of a rodent, e.g., a mouse, a rat, etc. In some embodiments, a cell is a germline cell. In some embodiments, a fraction of and not all cells, e.g., cells of particular cell types or tissues or location, of a population of cells, a tissue or an animal comprise a nucleotide sequence (e.g., a mutation) associated with a condition, disorder or disease, and such fraction of cells are engineered to comprise an ADAR1 polypeptide or a characteristic portion thereof or engineered to comprise and/or express a polynucleotide whose sequence encodes an ADAR1 polypeptide or a characteristic portion thereof. In some embodiments, a collection of liver cells comprise a SERPINA1 mutation, e.g., 1024 G>A (E342K) and a polynucleotide whose sequence encodes an ADAR1 polypeptide or a characteristic portion thereof. Those skilled in the art appreciate that various technologies are available for optionally controlled introduction and/or expression of a nucleotide sequence in various cells, tissues, or organs and can be utilized in accordance with the present disclosure. In some embodiments, as described herein, a cell, tissue or animal comprises a polynucleotide whose sequence encodes an ADAR1 polypeptide or a characteristic portion thereof in a genome, in some embodiments, in a germline genome. In some embodiments, as described herein, a cell, tissue or animal comprises a nucleotide sequence (e.g., a mutation) associated with a condition, disorder or disease in a genome, in some embodiments, in a germline genome.

As described herein, in some embodiments, a polynucleotide encodes human ADAR1 p110 or a characteristic portion thereof. In some embodiments, a polynucleotide encodes human ADAR1 p110. In some embodiments, a polynucleotide encodes human ADAR1 p150 or a characteristic portion thereof. In some embodiments, a polynucleotide encodes human ADAR1 p150. In some embodiments, a cell, tissue or animal (e.g., a huADAR mouse or a cell or tissue therefrom) is engineered to comprise and/or express a polynucleotide whose sequence encodes a human ADAR1 p110 polypeptide or a characteristic portion thereof. In some embodiments, a cell, tissue or animal (e.g., a huADAR mouse or a cell or tissue therefrom) is engineered to comprise and/or express a polynucleotide whose sequence encodes a human ADAR1 p110 polypeptide. In some embodiments, a cell, tissue or animal (e.g., a huADAR mouse or a cell or tissue therefrom) is engineered to comprise and/or express a polynucleotide whose sequence encodes a human ADAR1 p150 polypeptide or a characteristic portion thereof. In some embodiments, a cell, tissue or animal (e.g., a huADAR mouse or a cell or tissue therefrom) is engineered to comprise and/or express a polynucleotide whose sequence encodes a human ADAR1 p150 polypeptide. As described herein, in some embodiments, an animal is a rodent, e.g., a mouse or a rat.

In some embodiments, ADAR (e.g., human ADAR1) transgene is established on a zygote, e.g., SERPINA1 mouse zygote comprising a mutation (e.g., 1024 G>A (E342K) in human SERPINA1) or vice versa. In some embodiments, a zygote is homozygous. In some embodiments, a zygote is heterozygous.

### Methods of Using Provided Non-Human Animals, Cells or Tissues

Non-human animals (e.g., rodents, e.g., rats or mice), non-human (e.g., rodent, e.g., rat or mouse) cells and non-human (e.g., rodent, e.g., rat or mouse) tissues as described herein can be used as a platform for the development of therapeutic agents, e.g., oligonucleotides. In particular, non-human animals, non-human cells and non-human tissues as described herein represent a particularly advantageous platform for the identification and characterization of agents, e.g., oligonucleotides suitable for adenosine editing.

In some embodiments, the present disclosure provides that non-human animals (e.g., rodents, e.g., rats or mice), non-human (e.g., rodent, e.g., rat or mouse) cells and non-human (e.g., rodent, e.g., rat or mouse) tissues described herein can be used in methods characterizing/assessing various agents, e.g., oligonucleotides, and compositions thereof for adenosine editing (e.g., A to I). In some embodiments, a composition is an oligonucleotide composition. In some embodiments, oligonucleotides comprise various modifications, e.g., base, sugar, internucleotidic linkage modifications, etc. In some embodiments, linkage phosphorus in a modified internucleotidic linkage, e.g., a phosphorothioate internucleotidic linkage, is chiral (as appreciated by those skilled in the art, natural phosphate linkages commonly found in natural DNA and RNA molecules are achiral). In some embodiments, for various biological or therapeutic uses oligonucleotides comprise extensive modifications, and in some cases, contain no natural RNA sugars for, e.g., improved stability. In some embodiments, a composition is a stereorandom oligonucleotide composition. In some embodiments, a composition is a chirally controlled oligonucleotide composition, wherein one or more or all chiral linkage phosphorus are independently chirally controlled.

### Utilizing Non-Human Animals for Agent Assessment In Vivo

In some embodiments, non-human animals (e.g., rodents, e.g., rats or mice), non-human (e.g., rodent, e.g., rat or mouse) cells and non-human (e.g., rodent, e.g., rat or mouse) tissues as described herein may be employed for characterizing an oligonucleotide in vivo, wherein the expression of exogenous ADAR1 gene in said non-human animal provides an improved characterization platform when compared to a WT non-human animal (e.g., rodents, e.g., rats or mice).

In some embodiments, a non-human animal (e.g., genetically modified rodent, e.g., genetically modified rat or mouse) as described herein is treated (e.g., injected) with an oligonucleotide of interest under conditions and for a time sufficient that the non-human animal develops and/or has the potential to develop an ADAR mediated response to said oligonucleotide of interest. In some embodiments, injection may be but is not limited to:

In some embodiments, a non-human animal (e.g., genetically modified rodent, e.g., genetically modified rat or mouse) as described herein is treated (e.g., injected) with an oligonucleotide of interest under conditions and for a time sufficient that the non-human animal develops and/or has the potential to develop an ADAR mediated response to said oligonucleotide of interest. Sequences of RNA molecules (e.g., targets of an oligonucleotide of interest) are isolated and/or identified from the treated non-human animal (or one or more cells, for example, one or more B cells) and characterized using various assays measuring, for example, affinity, specificity, editing levels, transcript stability, translational efficiency, protein binding partners, nuclear localization, etc. In various embodiments, oligonucleotides characterized using non-human animals, non-human cells and/or non-human tissues as described herein comprise one or more regions that facilitate targeting of an endogenous loci of interest.

In some embodiments, a non-human animal (e.g., genetically modified rodent, e.g., genetically modified rat or mouse) as described herein is treated with an oligonucleotide of interest and the effects of said oligonucleotide in specific tissues are monitored and/or assessed.

### Utilizing Non-Human Animal Derived Cells for Agent Assessment In Vitro

In some embodiments, non-human (e.g., rodent, e.g., rat or mouse) cells as described herein comprising a transgenic ADAR1 locus may be employed for methods of characterizing potentially therapeutically efficacious oligonucleotides, the method comprising: (a) expressing in a non-human cell: (i) a first nucleotide sequence comprising a human ADAR1 gene; (ii) optionally additional nucleotide sequence comprising a human diseases locus of interest. and (b) introducing to a non-human cell: (i) a first exogenous oligonucleotide with the potential for site-directed RNA editing at a specific RNA locus mediated by an expressed ADAR gene; (ii) optionally additional exogenous oligonucleotide(s) with the potential for site-directed RNA editing at specific RNA loci mediated by an expressed ADAR gene.

In some embodiments, non-human (e.g., rodent, e.g., rat or mouse) cells as described herein comprising a transgenic ADAR1 locus may be employed for methods of characterizing potentially therapeutically efficacious oligonucleotides, the method comprising characterization in cells derived from the ADAR1 transgenic mouse. In some embodiments, such cells may be of any cell lineage and/or type of interest known in the art. In some embodiments, such cells may be but are not limited to: primary mouse hepatocytes, epidermal cells, epithelial cells, cortical neurons, sensory neurons, effector neurons, hormone-secreting cells, exocrine secretory epithelial cells, barrier cells, cardiomyocytes, leukocytes, lymphocytes, B cells, T cells, Bone Marrow cells, osteoblasts, chondrocytes, chondroblasts, adipocytes, cardiac muscle cells, muscle cells, fibroblasts, germ cells, nurse cells, kidney cells and/or an induced stem cell or product thereof derived from any of the aforementioned cells.

Non-human animals (e.g., rodents, e.g., rats or mice), non-human (e.g., rodent, e.g., rat or mouse) cells and non-human (e.g., rodent, e.g., rat or mouse) tissues as described herein may be employed for identifying oligonucleotides with potential to function as site-directed editing mediators.

Non-human animals (e.g., rodents, e.g., rats or mice), non-human (e.g., rodent, e.g., rat or mouse) cells and non-human (e.g., rodent, e.g., rat or mouse) tissues as described herein provide an improved in vivo system and source of biological materials (e.g., cells, nucleotides, polypeptides, protein complexes) for producing and characterizing oligonucleotides and/or polynucleotides that are useful for a variety of assays. In various embodiments, non-human animals, non-human cells and non-human tissues as described herein are used to develop therapeutics that target an RNA of interest (e.g., a RNA molecule known to function in a disease associated pathway) and/or modulate one or more activities associated with said RNA molecules of interest and/or modulate interactions of said RNA molecule of interest with other potential binding partners (e.g., any regulatory machinery that can act on intracellular RNA molecules, e.g., proteins and/or RNA species involved in translation, proteins and/or RNA species involved in innate immunity, proteins and/or RNA species involved in RNA interference, etc.,)

For example, in various embodiments, non-human animals, non-human cells and non-human tissues as described herein are used to develop therapeutics that target one or more receptor polypeptides, modulate receptor polypeptide activity and/or modulate receptor polypeptide interactions with other binding partners. In various embodiments, non-human animals, non-human cells and non-human tissues as described herein are used to identify, screen and/or develop candidate therapeutics (e.g., oligonucleotides) that bind to and facilitate ADAR mediated editing and/or regulation of one or more RNA molecules of interest.

In various embodiments, non-human animals, non-human cells and non-human tissues as described herein are used to screen and develop candidate therapeutics (e.g., oligonucleotides) that block activity of one or more RNA molecules of interest or that block the activity of one or more interactions between said RNA molecule of interest and other intracellular pathways.

In various embodiments, non-human animals (e.g., rodents, e.g., rats or mice), non-human (e.g., rodent, e.g., rat or mouse) cells and non-human (e.g., rodent, e.g., rat or mouse) tissues as described herein are used to determine the pharmacokinetic profiles of one or more oligonucleotide candidates. In various embodiments, one or more non-human animals, non-human cells and non-human tissues as described herein and one or more control or reference non-human animals, non-human cells and non-human tissues are each exposed to one or more agents, e.g., oligonucleotides at various doses (e.g., less than 0.1 mg/kg, 0.1 mg/kg, 0.2 mg/kg, 0.3 mg/kg, 0.4 mg/kg, 0.5 mg/kg, 1 mg/kg, 2 mg/kg, 3 mg/kg, 4 mg/kg, 5 mg/mg, 7.5 mg/kg, 10 mg/kg, 15 mg/kg, 20 mg/kg, 25 mg/kg, 30 mg/kg, 40 mg/kg, or 50 mg/kg or more). In some embodiments, oligonucleotides may be dosed to non-human animals at rates that vary as a function of gender, for example, in some embodiments a male animal may receive a higher dose than a comparable female animal, while in other embodiments, a female animal may receive a higher dose than a comparable male animal. In some embodiments, candidate therapeutic oligonucleotides may be dosed to non-human animals as described herein via any desired route of administration including parenteral and non-parenteral routes of administration. Parenteral routes include, e.g., intravenous, intra-arterial, intraportal, intramuscular, subcutaneous, intraperitoneal, intraspinal, intrathecal, intracerebroventricular, intracranial, intrapleural or other routes of injection. In some embodiments, administration may be non-parenteral, in some embodiments non-parenteral routes include, e.g., oral, nasal, transdermal, pulmonary, rectal, buccal, vaginal, ocular. In some embodiments, administration may also be by continuous infusion, local administration, sustained release from implants (gels, membranes or the like), and/or intravenous injection. In some embodiments, biological tissue (e.g., organs, blood, cells, secretions etc.) is isolated from non-human animals (humanized and control) at various time points (e.g., 0 hr, 6 hr, 1 day, 2 days, 3 days, 4 days, 5 days, 6 days, 7 days, 8 days, 9 days, 10 days, 11 days, or up to 30 or more days). Various assays may be performed to determine the pharmacokinetic profiles of administered candidate therapeutic oligonucleotides using samples obtained from non-human animals, non-human cells and non-human tissues as described herein including, but not limited to, editing levels, transcript levels, translational levels etc.

In various embodiments, non-human animals (e.g., rodents, e.g., rats or mice), non-human (e.g., rodent, e.g., rat or mouse) cells and non-human (e.g., rodent, e.g., rat or mouse) tissues as described herein are used to measure the therapeutic effect of blocking or modulating the activity of an RNA molecule of interest and the effect on gene expression as a result of cellular changes thereof.

Cells from provided non-human animals (e.g., rodents, e.g., rats or mice) can be isolated and used on an ad hoc basis, or can be maintained in culture for many generations. In various embodiments, cells from a provided non-human animal are immortalized (e.g., via use of a virus) and maintained in culture indefinitely (e.g., in serial cultures).

In some embodiments, a non-human (e.g., rodent, e.g., rat or mouse) cell is a non-human lymphocyte. In some embodiments, a non-human cell is selected from a B cell, dendritic cell, macrophage, monocyte and a T cell. In some embodiments, a non-human cell is an immature B cell, a mature naive B cell, an activated B cell, a memory B cell, and/or a plasma cell.

In some embodiments, a non-human (e.g., rodent, e.g., rat or mouse) cell is a non-human embryonic stem (ES) cell. In some embodiments, a non-human ES cell is a rodent ES cell. In some certain embodiments, a rodent ES cell is a mouse ES cell and is from a 129 strain, C57BL strain, BALB/c or a mixture thereof. In some certain embodiments, a rodent embryonic stem cell is a mouse embryonic stem cell and is a mixture of 129 and C57BL strains. In some certain embodiments, a rodent embryonic stem cell is a mouse embryonic stem cell and is a mixture of 129, C57BL and BALB/c strains.

In some embodiments, use of a non-human (e.g., rodent, e.g., rat or mouse) ES cell as described herein to make a non-human animal is provided. In some certain embodiments, a non-human ES cell is a mouse ES cell and is used to make a mouse comprising exogenous ADAR1 as described herein. In some certain embodiments, a non-human ES cell is a rat ES cell and is used to make a rat comprising exogenous ADAR1 as described herein.

In some embodiments, a non-human (e.g., rodent, e.g., rat or mouse) tissue is selected from but not limited to adipose, bladder, brain, breast, bone marrow, eye, heart, intestine, kidney, liver, lung, lymph node, muscle, pancreas, plasma, serum, skin, spleen, stomach, thymus, testis, ovum, and/or a combination thereof.

In some embodiments, an immortalized cell made, generated, produced or obtained from an isolated non-human cell or tissue as described herein is provided.

In some embodiments, a non-human (e.g., rodent, e.g., rat or mouse) embryo made, generated, produced, or obtained from a non-human ES cell as described herein is provided. In some certain embodiments, a non-human embryo is a rodent embryo; in some embodiments, a mouse embryo; in some embodiments, a rat embryo.

Non-human animals (e.g, rodents, e.g., rats or mice) as described herein provide an in vivo system for the generation of variants of human antibody variable regions that binds a polypeptide of interest (e.g., human Vλ domain variants). Such variants include human antibody variable regions having a desired functionality, specificity, low cross-reactivity to a common epitope shared by two or more variants of a polypeptide of interest. In some embodiments, non-human animals as described herein are employed to characterize panels of oligonucleotides that contain a series of variant sequences allowing for targeted modification of an RNA molecule of interest. In some embodiments, said panels of oligonucleotides are screened for a desired or improved functionality.

In some embodiments, a non-human animal (e.g., rodent, e.g., rat or mouse), non-human (e.g., rodent, e.g., rat or mouse) cell or non-human (e.g., rodent, e.g., rat or mouse) tissue as described herein is provided for use in the manufacture and/or development of a drug (e.g., an oligonucleotide or fragment thereof) for therapy or diagnosis.

In some embodiments, a non-human animal (e.g., rodent, e.g., rat or mouse), non-human (e.g., rodent, e.g., rat or mouse) cell or non-human (e.g., rodent, e.g., rat or mouse) tissue as described herein is provided for use in the manufacture and/or development of a medicament for the treatment, prevention or amelioration of a disease, disorder or condition.

In some embodiments, use of a non-human animal (e.g., rodent, e.g., rat or mouse), non-human (e.g., rodent, e.g., rat or mouse) cell or non-human (e.g., rodent, e.g., rat or mouse) tissue as described herein in the manufacture and/or development of a drug or vaccine for use in medicine, such as use as a medicament, is provided.

In some embodiments, non-human animals (e.g., rodents, e.g., rats or mice) as described herein provide an in vivo system for the analysis and testing of a drug or vaccine. In various embodiments, a candidate drug or vaccine may be delivered to one or more non-human animals as described herein, followed by monitoring of the non-human animals to determine one or more phenotypic (e.g., grossly visible and/or molecularly detectable) responses to the drug or vaccine, the safety profile of the drug or vaccine, or the effect on a disease or condition and/or one or more symptoms of a disease or condition. Exemplary methods used to determine the safety profile include measurements of toxicity, optimal dose concentration, antibody (i.e., anti-drug) response, efficacy of the drug or vaccine and possible risk factors. Such drugs or vaccines may be improved and/or developed in such non-human animals.

Vaccine efficacy may be determined in a number of ways. Briefly, non-human animals (e.g., rodents, e.g., rats or mice) as described herein are vaccinated using methods known in the art and then challenged with a vaccine or a vaccine is administered to already-infected non-human animals. The response of a non-human animal(s) to a vaccine may be measured by monitoring of, and/or performing one or more assays on, the non-human animal(s) (or cells isolated therefrom) to determine the efficacy of the vaccine. The response of a non-human animal(s) to the vaccine is then compared with control animals, using one or more measures known in the art and/or described herein.

Vaccine efficacy may further be determined by viral neutralization assays. Briefly, non-human animals (e.g., rodents, e.g., rats or mice) as described herein are immunized and serum is collected on various days post-immunization. Serial dilutions of serum are pre-incubated with a virus during which time antibodies in the serum that are specific for the virus will bind to it. The virus/serum mixture is then added to permissive cells to determine infectivity by a plaque assay or microneutralization assay. If antibodies in the serum neutralize the virus, there are fewer plaques or lower relative luciferase units compared to a control group.

In some embodiments, provided animals, cells, tissues, etc., are useful for manufacturing commercial batches of agents and compositions thereof. In some embodiments, the present disclosure provides a method comprising:
administering an agent or a composition thereof to a cell or a population thereof; and
assessing editing of an adenosine in a transcript in a cell or a population thereof.

In some embodiments, the present disclosure provides a method comprising:
administering an agent or a composition thereof to an animal or a population thereof; and
assessing editing of an adenosine in a transcript in one or more cells or tissues of the animal or a population thereof.

In some embodiments, the present disclosure provides a method comprising:
administering an oligonucleotide composition to a cell or a population thereof; and
assessing editing of an adenosine in a transcript in a cell or a population thereof.

In some embodiments, the present disclosure provides a method comprising:
administering an oligonucleotide composition to an animal or a population thereof; and
assessing editing of an adenosine in a transcript in one or more cells or tissues of the animal or a population thereof.

In some embodiments, cells or populations thereof are grown in vitro, e.g., in cell cultures. In some embodiments, agents or compositions are administered to cells or populations thereof of animals. In some embodiments, after administration cells or tissues are isolated from animals for assessing editing levels. In some embodiments, cells or tissues are associated with or are of conditions, disorders or diseases. In some embodiments, a single dose is administered. In some embodiments, two or more doses are administered with the same or different suitable time periods between doses. In some embodiments, assessing is performed after a suitable time period of a dosing. In some embodiments, multiple samples are analyzed for editing levels. In some embodiments, samples from various points (e.g., different time points after a dose and/or after different numbers of doses ) are assessed.

In some embodiments, an agent or a composition being administered is from a batch that is not a commercial batch. In some embodiments, it is from a batch that before the first commercial batch. In some embodiments, it is from a batch prepared for in vitro assessment of an agent or a composition and/or for assessment in an animal model. In some embodiments, it is determined that an agent or composition can provide a sufficient level of editing from assessing. In some embodiments, an agent or composition is manufactured or obtained after assessing, e.g., as commercial batches, on commercial scales, as drug products, etc. and/or for release, delivery and/or administration to subjects (e.g., human subjects).

In some embodiments, an agent or a composition being administered is from a commercial batch, e.g., one obtained or manufactured on a commercial scale. In some embodiments, an agent or a composition being administered is from a drug product, e.g., one suitable for administration to a subject, e.g., a human project. In some embodiments, editing level of an adenosine, e.g., a G->A mutation associated with a condition, disorder or disease as described herein, are compared to that of another batch of an agent or composition. In some embodiments, a level is compared to that of a non-commercial production prior to the first commercial production (e.g., a production utilized in early stages of development). In some embodiments, a level is compared to that of another commercial batch. In some embodiments, a level is compared to that of a reference sample or drug product. In some embodiments, a level is compared to that of another batch of a drug product. In some embodiments, a level is compared to a reference range. In some embodiments, a reference range is a range utilized to maintain relevant consistency of editing activity of multiple batches/preparations of an agent or a composition. In some embodiments, a reference range is derived from one or more batches of commercial production and/or drug product. In some embodiments, a reference range is derived from one or more batches of commercial production and/or drug product and/or pre-commercial batches (e.g., those during early research and development stages). In some embodiments, a reference range is about 10%-100%, 20%-100%, 30%-100%, 40%-100%, 50%-100%, 60%-100%, 70%-100%, 80-100%, 90-100%, 95-100%, 20%-90%, 30%-90%, 40%-90%, 50%-90%, 60%-90%, 70%-90%, 80-90%, 20%-85%, 30%-85%, 40%-85%, 50%-85%, 60%-85%, 70%-85%, 80-85%, 20%-80%, 30%-80%, 40%-80%, 50%-80%, 60%-80%, 70%-80%, 20%-75%, 30%-75%, 40%-75%, 50%-75%, 60%-75%, 70%-75%, or about +/-(about 1%, 2%, 3%, 4%, 5%, 6%, 7%, 8%, 9% or 10%) of 10%, 20%, 30%, 40%, 50%, 60%, 70%, 80%, 85%, 90%, 91%, 92%, 93%, 94%, 95%, 96%, 97%, 98%, 99%, or 100%, or at least about 10%, 20%, 30%, 40%, 50%, 60%, 70%, 80%, 85%, 90%, 91%, 92%, 93%, 94%, 95%, 96%, 97%, 98%, or 99%. In some embodiments, when editing level of an adenosine is comparable to a level being compared to, or is within a reference range, a batch of commercial production or drug product is released, e.g., for delivery, further procession (e.g., packaging), distribution, administration (e.g., to a subject such as a human subject), etc. In some embodiments, when editing level of an adenosine is not comparable to a level being compared to, or is outside a reference range, a batch of commercial production or drug product is rejected for delivery, distribution, administration, etc., is withheld (e.g., for further processing or to be destroyed), or is destroyed.

As described herein, in some embodiments, cells or animals are non-human cells or animals. In some embodiments, cells or animals are engineered non-human cells or animals, e.g., those engineered to comprise or express an ADAR1 polypeptide, e.g., hADAR1, or a fragment or a characteristic portion thereof as described herein. In some embodiments, cells are rodent cells. In some embodiments, animals are rodent animals. In some embodiments, a rodent is a mouse. In some embodiments, a rodent is a rat.

In some embodiments, agents are oligonucleotides. In some embodiments, compositions are oligonucleotide compositions. In some embodiments, compositions are stereorandom oligonucleotide compositions. In some embodiments, compositions are chirally controlled oligonucleotide compositions. In some embodiments, a chirally controlled oligonucleotide composition comprises a plurality of oligonucleotides, wherein each oligonucleotide of the plurality is independently a particular oligonucleotide or a salt thereof, and about 1%-100% (e.g., about 5%-100%, 10%-100%, 20%-100%, 30%-100%, 40%-100%, 50%-100%, 60%-100%, 70%-100%, 80-100%, 90-100%, 95-100%, 20%-90%, 30%-90%, 40%-90%, 50%-90%, 60%-90%, 70%-90%, 80-90%, 20%-85%, 30%-85%, 40%-85%, 50%-85%, 60%-85%, 70%-85%, 80-85%, 20%-80%, 30%-80%, 40%-80%, 50%-80%, 60%-80%, 70%-80%, 20%-75%, 30%-75%, 40%-75%, 50%-75%, 60%-75%, 70%-75%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in the composition that have the constitution of the particular oligonucleotide or a salt thereof are oligonucleotides of the plurality. In some embodiments, a chirally controlled oligonucleotide composition comprises a plurality of oligonucleotides that share the same constitution, wherein about 1%-100% (e.g., about 5%-100%, 10%-100%, 20%-100%, 30%-100%, 40%-100%, 50%-100%, 60%-100%, 70%-100%, 80-100%, 90-100%, 95-100%, 20%-90%, 30%-90%, 40%-90%, 50%-90%, 60%-90%, 70%-90%, 80-90%, 20%-85%, 30%-85%, 40%-85%, 50%-85%, 60%-85%, 70%-85%, 80-85%, 20%-80%, 30%-80%, 40%-80%, 50%-80%, 60%-80%, 70%-80%, 20%-75%, 30%-75%, 40%-75%, 50%-75%, 60%-75%, 70%-75%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in the composition that share the same constitution are oligonucleotides of the plurality. In some embodiments, the percentage is about or is at least about (DS)^{nc}, wherein DS is 90%-100% (e.g., 95%-100%, 90%, 91%, 92%, 93%, 94%, 95%, 96%, 97%, 98%, 99% or 99.5% or more) and nc is the number of chiral internucleotidic linkages in a particular oligonucleotide. In some embodiments, nc is 5 or more (e.g., 5-30, 5-25, 5-20, 10-30, 10-25, 10-20, 15-30, 15-25, 15-20, about or at least about 5, 6, 7, 8, 9, 10, 11, 12, 13, 14, 15, 16, 17, 18, 19, 20, 21, 22, 23, 24, 25, 26, 27, 28, 29 or 30). In some embodiments, a percentage is about 10%-100%, 20%-100%, 30%-100%, 40%-100%, 50%-100%, 60%-100%, 70%-100%, 80-100%, 90-100%, 95-100%, 20%-90%, 30%-90%, 40%-90%, 50%-90%, 60%-90%, 70%-90%, 80-90%, 20%-85%, 30%-85%, 40%-85%, 50%-85%, 60%-85%, 70%-85%, 80-85%, 20%-80%, 30%-80%, 40%-80%, 50%-80%, 60%-80%, 70%-80%, 20%-75%, 30%-75%, 40%-75%, 50%-75%, 60%-75%, 70%-75%, or about 10%, 20%, 30%, 40%, 50%, 60%, 70%, 80%, 85%, 90%, 91%, 92%, 93%, 94%, 95%, 96%, 97%, 98%, 99%, or 100%, or at least 10%, 20%, 30%, 40%, 50%, 60%, 70%, 80%, 85%, 90%, 91%, 92%, 93%, 94%, 95%, 96%, 97%, 98%, or 99%. In some embodiments, a chirally controlled oligonucleotide composition comprises an oligonucleotide or a salt thereof, wherein among all oligonucleotides in the composition that share the constitution of the particular oligonucleotide or a salt thereof, about or at least about 90% (e.g., 90-100%, 95%-100%, 90%, 91%, 92%, 93%, 94%, 95%, 96%, 97%, 98%, 99% or 99.5% or more) of such oligonucleotides share the same linkage phosphorus configuration as the particular oligonucleotide. In some embodiments, a chirally controlled oligonucleotide composition comprises an oligonucleotide, wherein one or more (e.g., 1-50, 5-30, 5-25, 5-20, 10-30, 10-25, 10-20, 15-30, 15-25, 15-20, about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30) or each chiral linkage phosphorus of the oligonucleotide independently has a diastereomeric ratio of about or at least about 90% (e.g., 90-100%, 95%-100%, 90%, 91%, 92%, 93%, 94%, 95%, 96%, 97%, 98%, 99% or 99.5% or more) (as used herein, x% = x:(100-x), e.g., 90% = 90:10. Diastereomeric ratio of a chiral center may be referred to as diastereomeric purity of a chiral center). In some embodiments, each chiral internucleotidic linkage has a diastereomeric ratio of about or at least about 90% (e.g., 90-100%, 95%-100%, 90%, 91%, 92%, 93%, 94%, 95%, 96%, 97%, 98%, 99% or 99.5% or more). Those skilled in the art appreciate that for a racemic oligonucleotide composition, diastereomeric ratio of each linkage phosphorus is typically about 50%, and the percentage of all oligonucleotides in the composition that share the constitution of an oligonucleotide (or a salt thereof) being the oligonucleotide (or a salt thereof) is about 2^{nc}. In some embodiments, compositions are pharmaceutically acceptable compositions.

### Kits

The present disclosure further provides a pack or kit comprising one or more containers filled with at least non-human cell, protein (single or complex (e.g., an antibody or fragment thereof)), DNA fragment, targeting vector, or any combination thereof, as described herein. Kits may be used in any applicable method (e.g., a research method). Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, and/or (c) a contract that governs the transfer of materials and/or biological products (e.g., a non-human animal or non-human cell as described herein) between two or more entities and combinations thereof.

In some embodiments, a kit comprising a non-human cell, non-human tissue, immortalized cell, non-human ES cell, or non-human embryo as described herein is provided. In some embodiments, a kit comprising an amino acid from a non-human animal, non-human cell, non-human tissue, immortalized cell, non-human ES cell, or non-human embryo as described herein is provided. In some embodiments, a kit comprising a nucleic acid (e.g., a nucleic acid encoding a human ADAR1 sequence described herein) from a non-human animal, non-human cell, non-human tissue, immortalized cell, non-human ES cell, or non-human embryo as described herein is provided. In some embodiments, a kit comprising a sequence (amino acid and/or nucleic acid sequence) identified from a non-human animal, non-human cell, non-human tissue, immortalized cell, non-human ES cell, or non-human embryo as described herein is provided.

In some embodiments, a kit as described herein for use in the manufacture and/or development of a drug (e.g., an oligonucleotide) for therapy or diagnosis is provided.

In some embodiments, a kit as described herein for use in the manufacture and/or development of a drug (e.g., an oligonucleotide) for the treatment, prevention or amelioration of a disease, disorder or condition is provided.

Other features of certain embodiments will become apparent in the course of the following descriptions of exemplary embodiments, which are given for illustration and are not intended to be limiting thereof.

Among other things, the present disclosure provides the following Embodiments:

1. A non-human animal engineered to comprise an ADAR1 polypeptide or a characteristic portion thereof.

2. A non-human animal engineered to comprise and/or express a polynucleotide whose sequence encodes an ADAR1 polypeptide or a characteristic portion thereof.

3. The animal of any one of the preceding Embodiments, wherein the genome of the animal comprises a polynucleotide whose sequence encodes an ADAR1 polypeptide or a characteristic portion thereof.

4. The animal of any one of the preceding Embodiments, wherein the germline genome of the animal comprises a polynucleotide whose sequence encodes an ADAR1 polypeptide or a characteristic portion thereof.

5. The animal of any one of the preceding Embodiments, wherein the polynucleotide comprises one or more introns.

6. The animal of any one of Embodiments 1-4, wherein the polynucleotide comprises no introns.

7. The animal of any one of the preceding Embodiments, wherein the animal is engineered to express an ADAR1 polypeptide or a characteristic portion thereof.

8. The animal of any one of the preceding Embodiments, wherein expression of the ADAR1 polypeptide or a characteristic portion thereof is inducible in one or more cells and/or tissues.

9. The animal of any one of the preceding Embodiments, wherein expression of the ADAR1 polypeptide or a characteristic portion thereof is constitutive in one or more cells and/or tissues.

10. The animal of any one of the preceding Embodiments, wherein expression of the ADAR1 polypeptide or a characteristic portion thereof is tissue specific.

11. The animal of any one of the preceding Embodiments, wherein the animal is a rodent.

12. The animal of any one of the preceding Embodiments, wherein the animal is a mouse.

13. The animal of any one of the preceding Embodiments, wherein the animal is a rat.

14. The animal of any one of the preceding Embodiments, wherein the ADAR1 polypeptide or a characteristic portion thereof is or comprises a deaminase domain.

15. The animal of any one of the preceding Embodiments, wherein the ADAR1 polypeptide or a characteristic portion thereof is or comprises one or more (e.g., 1, 2, 3 or more) dsRBDs.

16. The animal of any one of the preceding Embodiments, wherein the ADAR1 polypeptide or a characteristic portion thereof is or comprises one or more (e.g., 1, 2, 3 or more) Z-DNA binding domains.

17. The animal of any one of the preceding Embodiments, wherein the amino acid sequence of the ADAR1 polypeptide or a characteristic portion thereof is or comprises an amino acid sequence of a primate ADAR1 polypeptide or a fragment thereof.

18. The animal of any one of the preceding Embodiments, wherein the amino acid sequence of the ADAR1 polypeptide or a characteristic portion thereof is or comprises an amino acid sequence of a primate ADAR1 polypeptide or a characteristic portion thereof.

19. The animal of any one of the preceding Embodiments, wherein the amino acid sequence of the ADAR1 polypeptide or a characteristic portion thereof is or comprises an amino acid sequence of a primate ADAR1 polypeptide.

20. The animal of any one of the preceding Embodiments, wherein the amino acid sequence of the ADAR1 polypeptide or a characteristic portion thereof is an amino acid sequence of a primate ADAR1 polypeptide.

21. The animal of any one of the preceding Embodiments, wherein the ADAR1 polypeptide or a characteristic portion thereof is or comprises a primate ADAR1 polypeptide or a characteristic portion thereof.

22. The animal of any one of the preceding Embodiments, wherein the ADAR1 polypeptide or a characteristic portion thereof is a primate ADAR1 polypeptide.

23. The animal of any one of the preceding Embodiments, wherein the primate ADAR1 polypeptide is a human ADAR1 polypeptide.

24. The animal of any one of the preceding Embodiments, wherein the primate ADAR1 polypeptide is the p110 isoform of human ADAR1.

25. The animal of any one of Embodiments 1-23, wherein the primate ADAR1 polypeptide is the p150 isoform of human ADAR1.

26. The animal of any one of Embodiments 1-22, wherein the primate ADAR1 polypeptide is a non-human primate ADAR1 polypeptide.

27. The animal of any one of Embodiments 1-22, wherein the primate ADAR1 polypeptide is a monkey ADAR1 polypeptide.

28. The animal of any one of Embodiments 1-22, wherein the primate ADAR1 polypeptide is an ADAR1 polypeptide of cynomolgus macaques.

29. The animal of any one of the preceding Embodiments, wherein the ADAR1 polypeptide or a characteristic portion thereof, when contacted with an oligonucleotide composition targeting a target adenosine, provides increased editing level of the target adenosine (“engineered editing level”) in one or more cells or tissues of the engineered animal compared to that observed in the corresponding cells or tissues in a reference animal (“reference editing level”), wherein the reference animal is not engineered to express the ADAR1 polypeptide or a characteristic portion thereof.

30. The animal of any one of the preceding Embodiments, wherein the ADAR1 polypeptide or a characteristic portion thereof, when contacted with an oligonucleotide composition targeting a target adenosine, provides increased editing level of the target adenosine (“engineered editing level”) in one or more cells and/or tissues engineered to express the ADAR1 polypeptide or a characteristic portion thereof compared to that observed in reference cells and/or tissues not engineered to express the ADAR1 polypeptide or a characteristic portion thereof.

31. The animal of Embodiment 29 or 30, wherein the oligonucleotide composition is of WV-38700, and the target adenosine is its targeted adenosine in a human or mouse UGP2 transcript.

32. The animal of any one of Embodiments 29-31, wherein the oligonucleotide composition is of WV-38702, and the target adenosine is its targeted adenosine in a human or mouse UGP2 transcript.

33. The animal of any one of Embodiments 29-32, wherein the oligonucleotide composition is of WV- 40590, and the target adenosine is its targeted adenosine in a human or mouse UGP2 transcript.

34. The animal of any one of Embodiments 29-33, wherein the oligonucleotide composition is of WV- 40592, and the target adenosine is its targeted adenosine in a human or mouse SRSF1 transcript.

35. The animal of any one of Embodiments 29-34, wherein the oligonucleotide composition is of WV-38697, and the target adenosine is its targeted adenosine in a human or mouse EEF1A1 transcript.

36. The animal of any one of Embodiments 29-35, wherein the oligonucleotide composition is of WV-38699, and the target adenosine is its targeted adenosine in a human or mouse EEF1A1 transcript.

37. The animal of any one of Embodiments 29-36, wherein the one or more cells or tissues are liver tissue.

38. The animal of any one of Embodiments 29-36, wherein the one or more cells or tissues are mouse hepatocytes.

39. The animal of any one of the preceding Embodiments, wherein the engineered editing level is about or at least about 1.5, 2, 3, 4, 5, 6, 7, 8, 9, 10, 15, 20, 30, 40, 50, 100 or more fold of the reference editing level.

40. The animal of any one of the preceding Embodiments, wherein the engineered editing level is about or at least about 2 fold of the reference editing level.

41. The animal of any one of the preceding Embodiments, wherein the engineered editing level is about or at least about 5 fold of the reference editing level.

42. The animal of any one of the preceding Embodiments, wherein the engineered editing level is about or at least about 10 fold of the reference editing level.

43. The animal of any one of the preceding Embodiments, wherein the animal is heterozygous.

44. The animal of any one of Embodiments 1-43, wherein the animal is homozygous.

45. A non-human embryo engineered to comprise an ADAR1 polypeptide or a characteristic portion thereof.

46. A non-human embryo engineered to comprise and/or express a polynucleotide whose sequence encodes an ADARl polypeptide or a characteristic portion thereof.

47. The embryo of any one of the preceding Embodiments, wherein the genome of the embryo comprises a polynucleotide whose sequence encodes an ADAR1 polypeptide or a characteristic portion thereof.

48. The embryo of any one of the preceding Embodiments, wherein the polynucleotide comprises one or more introns.

49. The embryo of any one of Embodiments 45-47, wherein the polynucleotide comprises no introns.

50. The embryo of any one of the preceding Embodiments, wherein the embryo is engineered to express an ADAR1 polypeptide or a characteristic portion thereof.

51. The embryo of any one of the preceding Embodiments, wherein expression of the ADAR1 polypeptide or a characteristic portion thereof is inducible.

52. The embryo of any one of Embodiments 45-50, wherein expression of the ADAR1 polypeptide or a characteristic portion thereof is constitutive.

53. The embryo of any one of any one of the preceding Embodiments, wherein expression of the ADAR1 polypeptide or a characteristic portion thereof is operably linked to or regulated by one or more tissue-specific regulatory elements.

54. The embryo of any one of the preceding Embodiments, wherein the ADAR1 polypeptide or a characteristic portion thereof is or comprises a deaminase domain.

55. The embryo of any one of the preceding Embodiments, wherein the ADAR1 polypeptide or a characteristic portion thereof is or comprises one or more (e.g., 1, 2, 3 or more) dsRBDs.

56. The embryo of any one of the preceding Embodiments, wherein the ADAR1 polypeptide or a characteristic portion thereof is or comprises one or more (e.g., 1, 2, 3 or more) Z-DNA binding domains.

57. The embryo of any one of the preceding Embodiments, wherein the amino acid sequence of the ADAR1 polypeptide or a characteristic portion thereof is or comprises an amino acid sequence of a primate ADAR1 polypeptide or a fragment thereof.

58. The embryo of any one of the preceding Embodiments, wherein the amino acid sequence of the ADAR1 polypeptide or a characteristic portion thereof is or comprises an amino acid sequence of a primate ADAR1 polypeptide or a characteristic portion thereof.

59. The embryo of any one of the preceding Embodiments, wherein the amino acid sequence of the ADAR1 polypeptide or a characteristic portion thereof is or comprises an amino acid sequence of a primate ADAR1 polypeptide.

60. The embryo of any one of the preceding Embodiments, wherein the amino acid sequence of the ADAR1 polypeptide or a characteristic portion thereof is an amino acid sequence of a primate ADAR1 polypeptide.

61. The embryo of any one of the preceding Embodiments, wherein the ADAR1 polypeptide or a characteristic portion thereof is or comprises a primate ADAR1 polypeptide or a characteristic portion thereof.

62. The embryo of any one of the preceding Embodiments, wherein the ADAR1 polypeptide or a characteristic portion thereof is a primate ADAR1 polypeptide.

63. The embryo of any one of the preceding Embodiments, wherein the primate ADAR1 polypeptide is a human ADAR1 polypeptide.

64. The embryo of any one of the preceding Embodiments, wherein the primate ADAR1 polypeptide is the p110 isoform of human ADAR1.

65. The embryo of any one of Embodiments 45-63, wherein the primate ADAR1 polypeptide is the p 150 isoform of human ADAR1.

66. The embryo of any one of Embodiments 45-62, wherein the primate ADAR1 polypeptide is a non-human primate ADAR1 polypeptide.

67. The embryo of any one of Embodiments 45-62, wherein the primate ADAR1 polypeptide is a monkey ADAR1 polypeptide.

68. The embryo of any one of Embodiments 45-62, wherein the primate ADAR1 polypeptide is an ADAR1 polypeptide of cynomolgus macaques.

69. The embryo of any one of the preceding Embodiments, wherein the ADAR1 polypeptide or a characteristic portion thereof, when contacted with an oligonucleotide composition targeting a target adenosine, provides increased editing level of the target adenosine (“engineered editing level”) in one or more cells and/or tissues engineered to express the ADAR1 polypeptide or a characteristic portion thereof compared to that observed in reference cells and/or tissues not engineered to express the ADAR1 polypeptide or a characteristic portion thereof.

70. The embryo of Embodiment 69, wherein the oligonucleotide composition is of WV-38700, and the target adenosine is its targeted adenosine in a human or mouse UGP2 transcript.

71. The embryo of any one of Embodiments 69-70, wherein the oligonucleotide composition is of WV-38702, and the target adenosine is its targeted adenosine in a human or mouse UGP2 transcript.

72. The embryo of any one of Embodiments 69-71, wherein the oligonucleotide composition is of WV-40590, and the target adenosine is its targeted adenosine in a human or mouse UGP2 transcript.

73. The embryo of any one of Embodiments 69-72, wherein the oligonucleotide composition is of WV-40592, and the target adenosine is its targeted adenosine in a human or mouse SRSF1 transcript.

74. The embryo of any one of Embodiments 69-73, wherein the oligonucleotide composition is of WV-38697, and the target adenosine is its targeted adenosine in a human or mouse EEF1A1 transcript.

75. The embryo of any one of Embodiments 69-74, wherein the oligonucleotide composition is of WV-38699, and the target adenosine is its targeted adenosine in a human or mouse EEF1A1 transcript.

76. The embryo of any one of Embodiments 69-75, wherein the one or more cells or tissues are liver tissue.

77. The embryo of any one of Embodiments 69-75, wherein the one or more cells or tissues are mouse hepatocytes.

78. The embryo of any one of the preceding Embodiments, wherein the engineered editing level is about or at least about 1.5, 2, 3, 4, 5, 6, 7, 8, 9, 10, 15, 20, 30, 40, 50, 100 or more fold of the reference editing level.

79. The embryo of any one of the preceding Embodiments, wherein the engineered editing level is about or at least about 2 fold of the reference editing level.

80. The embryo of any one of the preceding Embodiments, wherein the engineered editing level is about or at least about 5 fold of the reference editing level.

81. The embryo of any one of the preceding Embodiments, wherein the engineered editing level is about or at least about 10 fold of the reference editing level.

82. The embryo of any one of the preceding Embodiments, wherein the embryo is heterozygous.

83. The embryo of any one of Embodiments 45-82, wherein the embryo is homozygous.

84. A cell engineered to comprise an ADAR1 polypeptide or a characteristic portion thereof.

85. A cell engineered to comprise and/or express a polynucleotide whose sequence encodes an ADAR1 polypeptide or a characteristic portion thereof.

86. The cell of any one of the preceding Embodiments, wherein the cell is a non-human cell.

87. The cell of any one of the preceding Embodiments, wherein the cell is an embryonic stem cell.

88. The cell of any one of the preceding Embodiments, wherein the genome of the cell comprises a polynucleotide whose sequence encodes an ADAR1 polypeptide or a characteristic portion thereof.

89. The cell of any one of the preceding Embodiments, wherein the polynucleotide comprises one or more introns.

90. The cell of any one of Embodiments 84-88, wherein the polynucleotide comprises no introns.

91. The cell of any one of the preceding Embodiments, wherein the cell is engineered to express an ADAR1 polypeptide or a characteristic portion thereof.

92. The cell of any one of the preceding Embodiments, wherein expression of the ADAR1 polypeptide or a characteristic portion thereof is inducible.

93. The cell of any one of Embodiments 84-91, wherein expression of the ADAR1 polypeptide or a characteristic portion thereof is constitutive.

94. The cell of any one of any one of the preceding Embodiments, wherein expression of the ADAR1 polypeptide or a characteristic portion thereof is operably linked to or regulated by one or more tissue-specific regulatory elements.

95. The cell of any one of the preceding Embodiments, wherein the ADAR1 polypeptide or a characteristic portion thereof is or comprises a deaminase domain.

96. The cell of any one of the preceding Embodiments, wherein the ADAR1 polypeptide or a characteristic portion thereof is or comprises one or more (e.g., 1, 2, 3 or more) dsRBMs.

97. The cell of any one of the preceding Embodiments, wherein the ADAR1 polypeptide or a characteristic portion thereof is or comprises one or more (e.g., 1, 2, 3 or more) Z-DNA binding domains.

98. The cell of any one of the preceding Embodiments, wherein the amino acid sequence of the ADAR1 polypeptide or a characteristic portion thereof is or comprises an amino acid sequence of a primate ADAR1 polypeptide or a fragment thereof.

99. The cell of any one of the preceding Embodiments, wherein the amino acid sequence of the ADAR1 polypeptide or a characteristic portion thereof is or comprises an amino acid sequence of a primate ADAR1 polypeptide or a characteristic portion thereof.

100. The cell of any one of the preceding Embodiments, wherein the amino acid sequence of the ADAR1 polypeptide or a characteristic portion thereof is or comprises an amino acid sequence of a primate ADAR1 polypeptide.

101. The cell of any one of the preceding Embodiments, wherein the amino acid sequence of the ADAR1 polypeptide or a characteristic portion thereof is an amino acid sequence of a primate ADAR1 polypeptide.

102. The cell of any one of the preceding Embodiments, wherein the ADAR1 polypeptide or a characteristic portion thereof is or comprises a primate ADAR1 polypeptide or a characteristic portion thereof.

103. The cell of any one of the preceding Embodiments, wherein the ADAR1 polypeptide or a characteristic portion thereof is a primate ADAR1 polypeptide.

104. The cell of any one of the preceding Embodiments, wherein the primate ADAR1 polypeptide is a human ADAR1 polypeptide.

105. The cell of any one of the preceding Embodiments, wherein the primate ADAR1 polypeptide is the p110 isoform of human ADAR1.

106. The cell of any one of Embodiments 84-104, wherein the primate ADAR1 polypeptide is the p 150 isoform of human ADAR1.

107. The cell of any one of Embodiments 84-103, wherein the primate ADAR1 polypeptide is a non-human primate ADAR1 polypeptide.

108. The cell of any one of Embodiments 84-103, wherein the primate ADAR1 polypeptide is a monkey ADAR1 polypeptide.

109. The cell of any one of Embodiments 84-103, wherein the primate ADAR1 polypeptide is an ADAR1 polypeptide of cynomolgus macaques.

110. The cell of any one of the preceding Embodiments, wherein the ADAR1 polypeptide or a characteristic portion thereof, when contacted with an oligonucleotide composition targeting a target adenosine, provides increased editing level of the target adenosine (“engineered editing level”) in one or more cells and/or tissues engineered to express the ADAR1 polypeptide or a characteristic portion thereof compared to that observed in reference cells and/or tissues not engineered to express the ADAR1 polypeptide or a characteristic portion thereof.

111. The cell of Embodiment 110, wherein the oligonucleotide composition is of WV-38700, and the target adenosine is its targeted adenosine in a human or mouse UGP2 transcript.

112. The cell of any one of Embodiments 110-111, wherein the oligonucleotide composition is of WV-38702, and the target adenosine is its targeted adenosine in a human or mouse UGP2 transcript.

113. The cell of any one of Embodiments 110-112, wherein the oligonucleotide composition is of WV-40590, and the target adenosine is its targeted adenosine in a human or mouse UGP2 transcript.

114. The cell of any one of Embodiments 110-113, wherein the oligonucleotide composition is of WV-40592, and the target adenosine is its targeted adenosine in a human or mouse SRSF1 transcript.

115. The cell of any one of Embodiments 110-114, wherein the oligonucleotide composition is of WV-38697, and the target adenosine is its targeted adenosine in a human or mouse EEF1A1 transcript.

116. The cell of any one of Embodiments 110-115, wherein the oligonucleotide composition is of WV-38699, and the target adenosine is its targeted adenosine in a human or mouse EEF1A1 transcript.

117. The cell of any one of Embodiments 110-116, wherein the one or more cells or tissues are liver tissue.

118. The cell of any one of Embodiments 110-116, wherein the one or more cells or tissues are mouse hepatocytes.

119. The cell of any one of the preceding Embodiments, wherein the engineered editing level is about or at least about 1.5, 2, 3, 4, 5, 6, 7, 8, 9, 10, 15, 20, 30, 40, 50, 100 or more fold of the reference editing level.

120. The cell of any one of the preceding Embodiments, wherein the engineered editing level is about or at least about 2 fold of the reference editing level.

121. The cell of any one of the preceding Embodiments, wherein the engineered editing level is about or at least about 5 fold of the reference editing level.

122. The cell of any one of the preceding Embodiments, wherein the engineered editing level is about or at least about 10 fold of the reference editing level.

123. The cell of any one of the preceding Embodiments, wherein the cell is heterozygous.

124. The cell of any one of Embodiments 84-122, wherein the cell is homozygous.

125. The animal, embryo or cell of any one of the preceding Embodiments, wherein the amino acid sequence of an ADAR1 polypeptide or a characteristic portion thereof is or comprises a sequence that is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with, SEQ ID NO: 27.

126. The animal, embryo or cell of any one of the preceding Embodiments, wherein the amino acid sequence of an ADAR1 polypeptide or a characteristic portion thereof is or comprises a sequence that is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with, SEQ ID NO: 28.

127. The animal, embryo or cell of any one of the preceding Embodiments, wherein the amino acid sequence of an ADAR1 polypeptide or a characteristic portion thereof is or comprises a sequence that is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with, SEQ ID NO: 29.

128. The animal, embryo or cell of any one of the preceding Embodiments, wherein the amino acid sequence of an ADAR1 polypeptide or a characteristic portion thereof is or comprises a sequence that is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with, SEQ ID NO: 30.

129. The animal, embryo or cell of any one of the preceding Embodiments, wherein the amino acid sequence of an ADAR1 polypeptide or a characteristic portion thereof is or comprises a sequence that is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with, SEQ ID NO: 31.

130. The animal, embryo or cell of any one of the preceding Embodiments, wherein the amino acid sequence of an ADAR1 polypeptide or a characteristic portion thereof is or comprises a sequence that is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with, SEQ ID NO: 32.

131. The animal, embryo or cell of any one of the preceding Embodiments, wherein the amino acid sequence of an ADAR1 polypeptide or a characteristic portion thereof is or comprises a sequence that is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with, SEQ ID NO: 33.

132. The animal, embryo or cell of any one of the preceding Embodiments, wherein the amino acid sequence of an ADAR1 polypeptide or a characteristic portion thereof is or comprises a sequence that is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with, SEQ ID NO: 34.

133. The animal, embryo or cell of any one of the preceding Embodiments, wherein the amino acid sequence of an ADAR1 polypeptide or a characteristic portion thereof is or comprises a sequence that is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with, SEQ ID NO: 35.

134. The animal, embryo or cell of any one of the preceding Embodiments, wherein the amino acid sequence of an ADAR1 polypeptide or a characteristic portion thereof is or comprises a sequence that is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with, SEQ ID NO: 36.

135. The animal, embryo or cell of any one of the preceding Embodiments, wherein the amino acid sequence of an ADAR1 polypeptide or a characteristic portion thereof is or comprises a sequence that is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with, SEQ ID NO: 37.

136. The animal, embryo or cell of any one of the preceding Embodiments, wherein the amino acid sequence of an ADAR1 polypeptide or a characteristic portion thereof is or comprises a sequence that is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with, SEQ ID NO: 38.

137. The animal, embryo or cell of any one of the preceding Embodiments, wherein the amino acid sequence of an ADAR1 polypeptide or a characteristic portion thereof is or comprises a sequence that is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with, SEQ ID NO: 39.

138. The animal, embryo or cell of any one of the preceding Embodiments, wherein the amino acid sequence of an ADAR1 polypeptide or a characteristic portion thereof is or comprises a sequence that is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with, SEQ ID NO: 40.

139. The animal, embryo or cell of any one of the preceding Embodiments, wherein the amino acid sequence of an ADAR1 polypeptide or a characteristic portion thereof is or comprises SEQ ID NO: 27.

140. The animal, embryo or cell of any one of the preceding Embodiments, wherein the amino acid sequence of an ADAR1 polypeptide or a characteristic portion thereof is or comprises SEQ ID NO: 28.

141. The animal, embryo or cell of any one of the preceding Embodiments, wherein the amino acid sequence of an ADAR1 polypeptide or a characteristic portion thereof is or comprises SEQ ID NO: 29.

142. The animal, embryo or cell of any one of the preceding Embodiments, wherein the amino acid sequence of an ADAR1 polypeptide or a characteristic portion thereof is or comprises SEQ ID NO: 30.

143. The animal, embryo or cell of any one of the preceding Embodiments, wherein the amino acid sequence of an ADAR1 polypeptide or a characteristic portion thereof is or comprises SEQ ID NO: 31.

144. The animal, embryo or cell of any one of the preceding Embodiments, wherein the amino acid sequence of an ADAR1 polypeptide or a characteristic portion thereof is or comprises SEQ ID NO: 32.

145. The animal, embryo or cell of any one of the preceding Embodiments, wherein the amino acid sequence of an ADAR1 polypeptide or a characteristic portion thereof is or comprises SEQ ID NO: 33.

146. The animal, embryo or cell of any one of the preceding Embodiments, wherein the amino acid sequence of an ADAR1 polypeptide or a characteristic portion thereof is or comprises SEQ ID NO: 34.

147. The animal, embryo or cell of any one of the preceding Embodiments, wherein the amino acid sequence of an ADAR1 polypeptide or a characteristic portion thereof is or comprises SEQ ID NO: 35.

148. The animal, embryo or cell of any one of the preceding Embodiments, wherein the amino acid sequence of an ADAR1 polypeptide or a characteristic portion thereof is or comprises SEQ ID NO: 36.

149. The animal, embryo or cell of any one of the preceding Embodiments, wherein the amino acid sequence of an ADAR1 polypeptide or a characteristic portion thereof is or comprises SEQ ID NO: 37.

150. The animal, embryo or cell of any one of the preceding Embodiments, wherein the amino acid sequence of an ADAR1 polypeptide or a characteristic portion thereof is or comprises SEQ ID NO: 38.

151. The animal, embryo or cell of any one of the preceding Embodiments, wherein the amino acid sequence of an ADAR1 polypeptide or a characteristic portion thereof is or comprises SEQ ID NO: 39.

152. The animal, embryo or cell of any one of the preceding Embodiments, wherein the amino acid sequence of an ADAR1 polypeptide or a characteristic portion thereof is or comprises SEQ ID NO: 40.

153. The animal, embryo or cell of any one of the preceding Embodiments, wherein the amino acid sequence of an ADAR1 polypeptide or a characteristic portion thereof is or comprises SEQ ID NO: 5 or a characteristic portion thereof.

154. The animal, embryo or cell of any one of the preceding Embodiments, wherein the amino acid sequence of an ADAR1 polypeptide or a characteristic portion thereof is or comprises SEQ ID NO: 6 or a characteristic portion thereof.

155. The animal, embryo or cell of any one of the preceding Embodiments, wherein the amino acid sequence of an ADAR1 polypeptide or a characteristic portion thereof is or comprises SEQ ID NO: 9 or a characteristic portion thereof.

156. The animal, embryo or cell of any one of the preceding Embodiments, wherein the amino acid sequence of an ADAR1 polypeptide or a characteristic portion thereof is or comprises SEQ ID NO: 12 or a characteristic portion thereof.

157. The animal, embryo or cell of any one of the preceding Embodiments, wherein the amino acid sequence of an ADAR1 polypeptide or a characteristic portion thereof is or comprises SEQ ID NO: 16 or a characteristic portion thereof.

158. The animal, embryo or cell of any one of the preceding Embodiments, wherein the amino acid sequence of an ADAR1 polypeptide or a characteristic portion thereof is or comprises SEQ ID NO: 20 or a characteristic portion thereof.

159. The animal, embryo or cell of any one of the preceding Embodiments, wherein the amino acid sequence of an ADAR1 polypeptide or a characteristic portion thereof is or comprises SEQ ID NO: 26 or a characteristic portion thereof.

160. The animal, embryo or cell of any one of the preceding Embodiments, wherein the amino acid sequence of an ADAR1 polypeptide or a characteristic portion thereof is or comprises SEQ ID NO: 43 or a characteristic portion thereof.

161. The animal, embryo or cell of any one of the preceding Embodiments, wherein the amino acid sequence of an ADAR1 polypeptide or a characteristic portion thereof is or comprises SEQ ID NO: 46 or a characteristic portion thereof.

162. The animal, embryo or cell of any one of the preceding Embodiments, wherein the amino acid sequence of an ADAR1 polypeptide or a characteristic portion thereof is or comprises SEQ ID NO: 49 or a characteristic portion thereof.

163. The animal, embryo or cell of any one of the preceding Embodiments, wherein the amino acid sequence of an ADAR1 polypeptide or a characteristic portion thereof is or comprises SEQ ID NO: 52 or a characteristic portion thereof.

164. The animal, embryo or cell of any one of the preceding Embodiments, wherein the amino acid sequence of an ADAR1 polypeptide or a characteristic portion thereof is or comprises SEQ ID NO: 55 or a characteristic portion thereof.

165. The animal, embryo or cell of any one of the preceding Embodiments, wherein the animal, embryo or cell is or comprises a cell, tissue or organ associated with or of a condition, disorder or disease.

166. The animal, embryo or cell of Embodiment 165, wherein a cell, tissue or organ associated with or of a condition, disorder or disease is or comprises a tumor.

167. The animal, embryo or cell of any one of the preceding Embodiments, wherein the animal, embryo or cell comprises a nucleotide sequence associated with a condition, disorder or disease.

168. The animal, embryo or cell of Embodiment 167, wherein a nucleotide sequence associated with a condition, disorder or disease is homozygous.

169. The animal, embryo or cell of Embodiment 167, wherein a nucleotide sequence associated with a condition, disorder or disease is heterozygous.

170. The animal, embryo or cell of Embodiment 167, wherein a nucleotide sequence associated with a condition, disorder or disease is hemizygous.

171. The animal, embryo or cell of any one of Embodiments 167-170, wherein a nucleotide sequence associated with a condition, disorder or disease is in a genome.

172. The animal, embryo or cell of any one of Embodiments 167-171, wherein a nucleotide sequence associated with a condition, disorder or disease is in a genome of some but not all cells.

173. The animal, embryo or cell of any one of Embodiments 167-172, wherein a nucleotide sequence associated with a condition, disorder or disease is in a germline genome.

174. The animal, embryo or cell of any one of Embodiments 167-173, wherein a nucleotide sequence associated with a condition, disorder or disease is a mutation.

175. The animal, embryo or cell of any one of the preceding Embodiments, wherein a nucleotide sequence associated with a condition, disorder or disease is a G to A mutation.

176. The animal, embryo or cell of any one of the preceding Embodiments, wherein a nucleotide sequence associated with a condition, disorder or disease is a G to A mutation in SERPINA1.

177. The animal, embryo or cell of any one of the preceding Embodiments, wherein the animal, embryo or cell comprises a G to A mutation that corresponds to 1024 G>A (E342K) mutation in human SERPINA1 gene.

178. The animal, embryo or cell of any one of the preceding Embodiments, wherein the animal, embryo or cell comprises a 1024 G>A (E342K) mutation in human SERPINA1 gene.

179. The animal, embryo or cell of any one of the preceding Embodiments, wherein the animal, embryo or cell comprises NOD.Cg-Prkdcscid Il2rgtm1Wjl Tg(SERPINA1*E342K)#Slew/SzJ.

180. The animal, embryo or cell of any one of the preceding Embodiments, wherein the animal, embryo or cell comprises a G to A mutation associated with a condition, disorder or disease.

181. A polynucleotide, comprising:
a) an ADAR1 polynucleotide encoding an ADAR1 polypeptide or a characteristic portion thereof,
b) a 5′ homology arm upstream of an ADAR1 polynucleotide, and
c) a 3′ homology arm downstream an ADAR1 polynucleotide,
wherein the 5′ and 3′ homology arms independently comprise a nucleotide sequence corresponding to a 5′ and 3′ target sequence in a non-human animal genome, respectively, wherein the 5′-target sequence is upstream of the 3′ target sequence.

182. The polynucleotide of Embodiment 181, wherein the 5′-target sequence is directly connected with the 3′-target sequence.

183. The polynucleotide of Embodiment 181, wherein there are about or less than about 0.1, 0.2, 0.3, 0.4, 0.5, 0.6, 0.7, 0.8, 0.9, 1, 2, 3, 4, 5, 6, 7, 8, 9, or 10 kb between the 5′- and 3′-target sequence.

184. The polynucleotide of any one of the preceding Embodiments, wherein the ADAR1 polynucleotide comprises one or more introns.

185. The polynucleotide of any one of Embodiments 181-183, wherein the ADAR1 polynucleotide comprises no introns.

186. The polynucleotide of any one of the preceding Embodiments, wherein the ADAR1 polynucleotide is codon optimized to express an ADAR1 polypeptide or a characteristic portion thereof in an animal host cell.

187. The polynucleotide of Embodiment 186, wherein the animal is a rodent.

188. The polynucleotide of Embodiment 186, wherein the animal is a mouse.

189. The polynucleotide of Embodiment 186, wherein the animal is a rat.

190. The polynucleotide of any one of the preceding Embodiments, wherein the polynucleotide comprises regulatory elements for inducible expression of the ADAR1 polynucleotide.

191. The polynucleotide of any one of Embodiments 181-189, wherein the polynucleotide comprises regulatory elements for constitutive expression of the ADAR1 polynucleotide.

192. The polynucleotide of any one of the preceding Embodiments, wherein the polynucleotide comprises regulatory elements for tissue-specific expression of the ADAR1 polynucleotide.

193. The polynucleotide of any one of the preceding Embodiments, wherein the ADAR1 polypeptide or a characteristic portion thereof is or comprises a deaminase domain.

194. The polynucleotide of any one of the preceding Embodiments, wherein the ADAR1 polypeptide or a characteristic portion thereof is or comprises one or more (e.g., 1, 2, 3 or more) dsRBMs.

195. The polynucleotide of any one of the preceding Embodiments, wherein the ADAR1 polypeptide or a characteristic portion thereof is or comprises one or more (e.g., 1, 2, 3 or more) Z-DNA binding domains.

196. The polynucleotide of any one of the preceding Embodiments, wherein the amino acid sequence of the ADAR1 polypeptide or a characteristic portion thereof is or comprises an amino acid sequence of a primate ADAR1 polypeptide or a fragment thereof.

197. The polynucleotide of any one of the preceding Embodiments, wherein the amino acid sequence of the ADAR1 polypeptide or a characteristic portion thereof is or comprises an amino acid sequence of a primate ADAR1 polypeptide or a characteristic portion thereof.

198. The polynucleotide of any one of the preceding Embodiments, wherein the amino acid sequence of the ADAR1 polypeptide or a characteristic portion thereof is or comprises an amino acid sequence of a primate ADAR1 polypeptide.

199. The polynucleotide of any one of the preceding Embodiments, wherein the amino acid sequence of the ADAR1 polypeptide or a characteristic portion thereof is an amino acid sequence of a primate ADAR1 polypeptide.

200. The polynucleotide of any one of the preceding Embodiments, wherein the ADAR1 polypeptide or a characteristic portion thereof is or comprises a primate ADAR1 polypeptide or a characteristic portion thereof.

201. The polynucleotide of any one of the preceding Embodiments, wherein the ADAR1 polypeptide or a characteristic portion thereof is a primate ADAR1 polypeptide.

202. The polynucleotide of any one of the preceding Embodiments, wherein the primate ADAR1 polypeptide is a human ADAR1 polypeptide.

203. The polynucleotide of any one of the preceding Embodiments, wherein the primate ADAR1 polypeptide is the p110 isoform of human ADAR1.

204. The polynucleotide of any one of Embodiments 181-202, wherein the primate ADAR1 polypeptide is the p 150 isoform of human ADAR1.

205. The polynucleotide of any one of Embodiments 181-201, wherein the primate ADAR1 polypeptide is a non-human primate ADAR1 polypeptide.

206. The polynucleotide of any one of Embodiments 181-201, wherein the primate ADAR1 polypeptide is a monkey ADAR1 polypeptide.

207. The polynucleotide of any one of Embodiments 181-201, wherein the primate ADAR1 polypeptide is an ADAR1 polypeptide of cynomolgus macaques.

208. The polynucleotide of any one of the preceding Embodiments, wherein the ADAR1 polypeptide or a characteristic portion thereof, when contacted with an oligonucleotide composition targeting a target adenosine, provides increased editing level of the target adenosine (“engineered editing level”) in one or more cells and/or tissues engineered to express the ADAR1 polypeptide or a characteristic portion thereof compared to that observed in reference cells and/or tissues not engineered to express the ADAR1 polypeptide or a characteristic portion thereof.

209. The polynucleotide of Embodiment 208, wherein the oligonucleotide composition is of WV-38700, and the target adenosine is its targeted adenosine in a human or mouse UGP2 transcript.

210. The polynucleotide of any one of Embodiments 208-209, wherein the oligonucleotide composition is of WV-38702, and the target adenosine is its targeted adenosine in a human or mouse UGP2 transcript.

211. The polynucleotide of any one of Embodiments 208-210, wherein the oligonucleotide composition is of WV-40590, and the target adenosine is its targeted adenosine in a human or mouse UGP2 transcript.

212. The polynucleotide of any one of Embodiments 208-211, wherein the oligonucleotide composition is of WV-40592, and the target adenosine is its targeted adenosine in a human or mouse SRSF1 transcript.

213. The polynucleotide of any one of Embodiments 208-212, wherein the oligonucleotide composition is of WV-38697, and the target adenosine is its targeted adenosine in a human or mouse EEF1A1 transcript.

214. The polynucleotide of any one of Embodiments 208-213, wherein the oligonucleotide composition is of WV-38699, and the target adenosine is its targeted adenosine in a human or mouse EEF1A1 transcript.

215. The polynucleotide of any one of Embodiments 208-214, wherein the one or more cells or tissues are liver tissue.

216. The polynucleotide of any one of Embodiments 208-214, wherein the one or more cells or tissues are mouse hepatocytes.

217. The polynucleotide of any one of the preceding Embodiments, wherein the polynucleotide has or comprises a sequence that encodes an ADAR1 polypeptide or a characteristic portion thereof of any one of the preceding Embodiments.

218. The polynucleotide of any one of the preceding Embodiments, wherein the sequence of the polynucleotide is or comprises SEQ ID NO: 2 or a characteristic portion thereof.

219. The polynucleotide of any one of the preceding Embodiments, wherein the sequence of the polynucleotide is or comprises SEQ ID NO: 3 or a characteristic portion thereof.

220. The polynucleotide of any one of the preceding Embodiments, wherein the sequence of the polynucleotide is or comprises SEQ ID NO: 4 or a characteristic portion thereof.

221. The polynucleotide of any one of the preceding Embodiments, wherein the sequence of the polynucleotide is or comprises SEQ ID NO: 7 or a characteristic portion thereof.

222. The polynucleotide of any one of the preceding Embodiments, wherein the sequence of the polynucleotide is or comprises SEQ ID NO: 8 or a characteristic portion thereof.

223. The polynucleotide of any one of the preceding Embodiments, wherein the sequence of the polynucleotide is or comprises SEQ ID NO: 10 or a characteristic portion thereof.

224. The polynucleotide of any one of the preceding Embodiments, wherein the sequence of the polynucleotide is or comprises SEQ ID NO: 11 or a characteristic portion thereof.

225. The polynucleotide of any one of the preceding Embodiments, wherein the sequence of the polynucleotide is or comprises SEQ ID NO: 13 or a characteristic portion thereof.

226. The polynucleotide of any one of the preceding Embodiments, wherein the sequence of the polynucleotide is or comprises SEQ ID NO: 14 or a characteristic portion thereof.

227. The polynucleotide of any one of the preceding Embodiments, wherein the sequence of the polynucleotide is or comprises SEQ ID NO: 15 or a characteristic portion thereof.

228. The polynucleotide of any one of the preceding Embodiments, wherein the sequence of the polynucleotide is or comprises SEQ ID NO: 17 or a characteristic portion thereof.

229. The polynucleotide of any one of the preceding Embodiments, wherein the sequence of the polynucleotide is or comprises SEQ ID NO: 18 or a characteristic portion thereof.

230. The polynucleotide of any one of the preceding Embodiments, wherein the sequence of the polynucleotide is or comprises SEQ ID NO: 19 or a characteristic portion thereof.

231. The polynucleotide of any one of the preceding Embodiments, wherein the sequence of the polynucleotide is or comprises SEQ ID NO: 21 or a characteristic portion thereof.

232. The polynucleotide of any one of the preceding Embodiments, wherein the sequence of the polynucleotide is or comprises SEQ ID NO: 22 or a characteristic portion thereof.

233. The polynucleotide of any one of the preceding Embodiments, wherein the sequence of the polynucleotide is or comprises SEQ ID NO: 23 or a characteristic portion thereof.

234. The polynucleotide of any one of the preceding Embodiments, wherein the sequence of the polynucleotide is or comprises SEQ ID NO: 24 or a characteristic portion thereof.

235. The polynucleotide of any one of the preceding Embodiments, wherein the sequence of the polynucleotide is or comprises SEQ ID NO: 25 or a characteristic portion thereof.

236. The polynucleotide of any one of the preceding Embodiments, wherein the sequence of the polynucleotide is or comprises SEQ ID NO: 41 or a characteristic portion thereof.

237. The polynucleotide of any one of the preceding Embodiments, wherein the sequence of the polynucleotide is or comprises SEQ ID NO: 42 or a characteristic portion thereof.

238. The polynucleotide of any one of the preceding Embodiments, wherein the sequence of the polynucleotide is or comprises SEQ ID NO: 44 or a characteristic portion thereof.

239. The polynucleotide of any one of the preceding Embodiments, wherein the sequence of the polynucleotide is or comprises SEQ ID NO: 45 or a characteristic portion thereof.

240. The polynucleotide of any one of the preceding Embodiments, wherein the sequence of the polynucleotide is or comprises SEQ ID NO: 47 or a characteristic portion thereof.

241. The polynucleotide of any one of the preceding Embodiments, wherein the sequence of the polynucleotide is or comprises SEQ ID NO: 48 or a characteristic portion thereof.

242. The polynucleotide of any one of the preceding Embodiments, wherein the sequence of the polynucleotide is or comprises SEQ ID NO: 50 or a characteristic portion thereof.

243. The polynucleotide of any one of the preceding Embodiments, wherein the sequence of the polynucleotide is or comprises SEQ ID NO: 51 or a characteristic portion thereof.

244. The polynucleotide of any one of the preceding Embodiments, wherein the sequence of the polynucleotide is or comprises SEQ ID NO: 53 or a characteristic portion thereof.

245. The polynucleotide of any one of the preceding Embodiments, wherein the sequence of the polynucleotide is or comprises SEQ ID NO: 54 or a characteristic portion thereof.

246. The polynucleotide of any one of the preceding Embodiments, wherein the engineered editing level is about or at least about 1.5, 2, 3, 4, 5, 6, 7, 8, 9, 10, 15, 20, 30, 40, 50, 100 or more fold of the reference editing level.

247. The polynucleotide of any one of the preceding Embodiments, wherein the engineered editing level is about or at least about 2 fold of the reference editing level.

248. The polynucleotide of any one of the preceding Embodiments, wherein the engineered editing level is about or at least about 5 fold of the reference editing level.

249. The polynucleotide of any one of the preceding Embodiments, wherein the engineered editing level is about or at least about 10 fold of the reference editing level.

250. The animal, embryo or cell of any one of the preceding Embodiments, comprising and/or expressing a polynucleotide of any one of the preceding Embodiments.

251. A vector comprising or expressing an ADAR1 polynucleotide encoding an ADAR1 polypeptide or a characteristic portion thereof.

252. A vector, comprising and/or expressing:
a) an ADAR1 polynucleotide encoding an ADAR1 polypeptide or a characteristic portion thereof,
b) a 5′ homology arm upstream of an ADAR1 polynucleotide, and
c) a 3′ homology arm downstream an ADAR1 polynucleotide,
wherein the 5′ and 3′ homology arms independently comprise a nucleotide sequence corresponding to a 5′ and 3′ target sequence in a non-human animal genome, respectively, wherein the 5′-target sequence is upstream of the 3′ target sequence.

253. The vector of Embodiment 252, wherein the 5′-target sequence is directly connected with the 3′-target sequence.

254. The vector of Embodiment 252, wherein there are about or less than about 0.1, 0.2, 0.3, 0.4, 0.5, 0.6, 0.7, 0.8, 0.9, 1, 2, 3, 4, 5, 6, 7, 8, 9, or 10 kb between the 5′- and 3′-target sequence.

255. The vector of any one of the preceding Embodiments, wherein the ADAR1 polynucleotide comprises one or more introns.

256. The vector of any one of Embodiments 251-254, wherein the ADAR1 polynucleotide comprises no introns.

257. The vector of any one of the preceding Embodiments, wherein the ADAR1 polynucleotide is codon optimized to express an ADAR1 polypeptide or a characteristic portion thereof in an animal host cell.

258. The vector of Embodiment 186, wherein the animal is a rodent.

259. The vector of Embodiment 186, wherein the animal is a mouse.

260. The vector of Embodiment 186, wherein the animal is a rat.

261. The vector of any one of the preceding Embodiments, wherein the vector comprises regulatory elements for inducible expression of the ADAR1 polynucleotide.

262. The vector of any one of Embodiments 251-260, wherein the vector comprises regulatory elements for constitutive expression of the ADAR1 polynucleotide.

263. The vector of any one of the preceding Embodiments, wherein the vector comprises regulatory elements for tissue-specific expression of the ADAR1 polynucleotide.

264. The vector of any one of the preceding Embodiments, wherein the ADAR1 polypeptide or a characteristic portion thereof is or comprises a deaminase domain.

265. The vector of any one of the preceding Embodiments, wherein the ADAR1 polypeptide or a characteristic portion thereof is or comprises one or more (e.g., 1, 2, 3 or more) dsRBMs.

266. The vector of any one of the preceding Embodiments, wherein the ADAR1 polypeptide or a characteristic portion thereof is or comprises one or more (e.g., 1, 2, 3 or more) Z-DNA binding domains.

267. The vector of any one of the preceding Embodiments, wherein the amino acid sequence of the ADAR1 polypeptide or a characteristic portion thereof is or comprises an amino acid sequence of a primate ADAR1 polypeptide or a fragment thereof.

268. The vector of any one of the preceding Embodiments, wherein the amino acid sequence of the ADAR1 polypeptide or a characteristic portion thereof is or comprises an amino acid sequence of a primate ADAR1 polypeptide or a characteristic portion thereof.

269. The vector of any one of the preceding Embodiments, wherein the amino acid sequence of the ADAR1 polypeptide or a characteristic portion thereof is or comprises an amino acid sequence of a primate ADAR1 polypeptide.

270. The vector of any one of the preceding Embodiments, wherein the amino acid sequence of the ADAR1 polypeptide or a characteristic portion thereof is an amino acid sequence of a primate ADAR1 polypeptide.

271. The vector of any one of the preceding Embodiments, wherein the ADAR1 polypeptide or a characteristic portion thereof is or comprises a primate ADAR1 polypeptide or a characteristic portion thereof.

272. The vector of any one of the preceding Embodiments, wherein the ADAR1 polypeptide or a characteristic portion thereof is a primate ADAR1 polypeptide.

273. The vector of any one of the preceding Embodiments, wherein the primate ADAR1 polypeptide is a human ADAR1 polypeptide.

274. The vector of any one of the preceding Embodiments, wherein the primate ADAR1 polypeptide is the p110 isoform of human ADAR1.

275. The vector of any one of Embodiments 251-273, wherein the primate ADAR1 polypeptide is the p 150 isoform of human ADAR1.

276. The vector of any one of Embodiments 251-272, wherein the primate ADAR1 polypeptide is a non-human primate ADAR1 polypeptide.

277. The vector of any one of Embodiments 251-272, wherein the primate ADAR1 polypeptide is a monkey ADAR1 polypeptide.

278. The vector of any one of Embodiments 251-272, wherein the primate ADAR1 polypeptide is an ADAR1 polypeptide of cynomolgus macaques.

279. The vector of any one of the preceding Embodiments, wherein the ADAR1 polypeptide or a characteristic portion thereof, when contacted with an oligonucleotide composition targeting a target adenosine, provides increased editing level of the target adenosine (“engineered editing level”) in one or more cells and/or tissues engineered to express the ADAR1 polypeptide or a characteristic portion thereof compared to that observed in reference cells and/or tissues not engineered to express the ADAR1 polypeptide or a characteristic portion thereof.

280. The vector of Embodiment 279, wherein the oligonucleotide composition is of WV-38700, and the target adenosine is its targeted adenosine in a human or mouse UGP2 transcript.

281. The vector of any one of Embodiments 279-280, wherein the oligonucleotide composition is of WV-38702, and the target adenosine is its targeted adenosine in a human or mouse UGP2 transcript.

282. The vector of any one of Embodiments 279-281, wherein the oligonucleotide composition is of WV-40590, and the target adenosine is its targeted adenosine in a human or mouse UGP2 transcript.

283. The vector of any one of Embodiments 279-282, wherein the oligonucleotide composition is of WV-40592, and the target adenosine is its targeted adenosine in a human or mouse SRSF1 transcript.

284. The vector of any one of Embodiments 279-283, wherein the oligonucleotide composition is of WV-38697, and the target adenosine is its targeted adenosine in a human or mouse EEF1A1 transcript.

285. The vector of any one of Embodiments 279-284, wherein the oligonucleotide composition is of WV-38699, and the target adenosine is its targeted adenosine in a human or mouse EEF1A1 transcript.

286. The vector of any one of Embodiments 279-285, wherein the one or more cells or tissues are liver tissue.

287. The vector of any one of Embodiments 279-285, wherein the one or more cells or tissues are mouse hepatocytes.

288. The vector of any one of the preceding Embodiments, wherein the engineered editing level is about or at least about 1.5, 2, 3, 4, 5, 6, 7, 8, 9, 10, 15, 20, 30, 40, 50, 100 or more fold of the reference editing level.

289. The vector of any one of the preceding Embodiments, wherein the engineered editing level is about or at least about 2 fold of the reference editing level.

290. The vector of any one of the preceding Embodiments, wherein the engineered editing level is about or at least about 5 fold of the reference editing level.

291. The vector of any one of the preceding Embodiments, wherein the engineered editing level is about or at least about 10 fold of the reference editing level.

292. The vector of any one of the preceding Embodiments, wherein the vector is a targeting vector. 293. A vector which comprises and/or expresses a polynucleotide of any one of the preceding Embodiments.

294. The animal, embryo or cell of any one of the preceding Embodiments, comprising a polynucleotide of any one of the preceding Embodiments.

295. The animal, embryo or cell of any one of the preceding Embodiments, expressing a polynucleotide of any one of the preceding Embodiments.

296. The animal, embryo or cell of any one of the preceding Embodiments, wherein the animal, embryo or cell provides a higher level of editing of an ADAR1 target adenosine (“engineered editing level”) compared to that in a reference animal, embryo or cell not engineered to express the ADAR1 polypeptide or a characteristic portion thereof (“reference editing level”).

297. The animal, embryo or cell of Embodiment 296, wherein the engineered editing level is about or at least about 1.5, 2, 3, 4, 5, 6, 7, 8, 9, 10, 15, 20, 30, 40, 50, 100 or more fold of the reference editing level.

298. The animal, embryo or cell of Embodiment 296, wherein the engineered editing level is about or at least about 2 fold of the reference editing level.

299. The animal, embryo or cell of Embodiment 296, wherein the engineered editing level is about or at least about 5 fold of the reference editing level.

300. The animal, embryo or cell of Embodiment 296, wherein the engineered editing level is about or at least about 10 fold of the reference editing level.

301. A population of cells, comprising a plurality of cells each of which is independently a cell of any one of the preceding Embodiments.

302. A population of cells, wherein each cell is independently a cell of any one of the preceding Embodiments.

303. A method, comprising introducing a polynucleotide or vector of any one of the preceding Embodiments into a cell, embryo or animal.

304. The method of Embodiment 303, wherein the cell, embryo or animal, prior to the introducing, does not comprise or express an ADAR1 polynucleotide.

305. The method of Embodiment 303, wherein the cell, embryo or animal, prior to the introducing, does not comprise or express a primate or human ADAR1 polynucleotide.

306. The method of any one of Embodiments 303-305, wherein the cell, embryo or animal is rodent cell embryo or animal.

307. The method of any one of Embodiments 303-305, wherein the cell, embryo or animal is mouse cell embryo or animal.

308. The method of any one of Embodiments 303-305, wherein the cell, embryo or animal is rat cell embryo or animal.

309. The method of any one of Embodiments 303-308, wherein the method provides a cell, embryo or animal of any one of the preceding Embodiments.

310. A method of making a non-human animal, comprising:
(a) introducing an ADAR1 polynucleotide encoding an ADAR1 polypeptide or a characteristic portion thereof into the genome of an ES cell of a non-human animal; and
(b) generating a non-human animal using the ES cell generated in step (a).

311. A method of making a non-human animal, comprising:
(a) introducing a polynucleotide of any one of the preceding Embodiments into the genome of an ES cell of a non-human animal; and
(b) generating a non-human animal using the ES cell generated in step (a).

312. A method of making a non-human animal, comprising:
(a) introducing an ADAR1 polynucleotide encoding an ADAR1 polypeptide or a characteristic portion thereof into the genome of a zygote of a non-human animal; and
(b) generating a non-human animal using the zygote generated in step (a).

313. A method of making a non-human animal, comprising:
(a) introducing a polynucleotide of any one of the preceding Embodiments into the genome of a zygote of a non-human animal; and
(b) generating a non-human animal using the zygote generated in step (a).

314. A method of making a genetically modified ES cell of a non-human animal comprising:
introducing an ADAR1 polynucleotide encoding an ADAR1 polypeptide or a characteristic portion thereof into the genome of an ES cell of a non-human animal.

315. A method of making a genetically modified ES cell of a non-human animal comprising:
introducing a polynucleotide of any one of the preceding Embodiments into the genome of an ES cell of a non-human animal.

316. A method for generating an engineered cell, comprising:
introducing an ADAR1 polynucleotide encoding an ADAR1 polypeptide or a characteristic portion thereof into a cell of a non-human animal.

317. A method for generating an engineered cell, comprising:
introducing a polynucleotide of any one of the preceding Embodiments into a cell of a non-human animal.

318. The method of any one of the preceding Embodiments, comprising steps of:
contacting the genome of a cell with a nuclease to create a DNA break; and
introducing a polynucleotide of any one of the preceding Embodiments.

319. The method of Embodiment 318, wherein the nuclease is CRISPR/Cas9.

320. The method of Embodiment 318, wherein the nuclease is a zinc finger nuclease.

321. The method of Embodiment 318, wherein the nuclease is a transcriptional activator-like effector nuclease.

322. A method for generating an animal, embryo or cell, comprising introducing to a first animal, embryo or cell a polynucleotide whose sequence encodes an ADAR1 polypeptide or a characteristic portion thereof.

323. The method of Embodiment 322, wherein an ADAR1 polypeptide is a human ADAR1.

324. The method of Embodiment 322, wherein an ADAR1 polypeptide is a human ADAR1 p 110.

325. The method of Embodiment 322, wherein an ADAR1 polypeptide is a human ADAR1 p150.

326. A method for generating an animal, embryo or cell, comprising breeding a first animal with a second animal, wherein the second animal is an animal of any one of the preceding Embodiments.

327. The method of any one of Embodiments 322-326, wherein a first animal, embryo or cell is or comprises a cell, tissue or organ associated with or of a condition, disorder or disease.

328. A method for generating an animal, embryo or cell, comprising introducing a first cell, tissue or organ associated with or of a condition, disorder or disease to a second animal, embryo or cell of any one of the preceding Embodiments.

329. The method of any one of Embodiments 327-328, wherein a cell, tissue or organ associated with or of a condition, disorder or disease is or comprises a tumor.

330. The method of any one of Embodiments 322-329, wherein a first animal, embryo or cell comprises a nucleotide sequence associated with a condition, disorder or disease.

331. The method of any one Embodiments 322-330, wherein a nucleotide sequence associated with a condition, disorder or disease is homozygous.

332. The method of any one Embodiments 322-330, wherein a nucleotide sequence associated with a condition, disorder or disease is heterozygous.

333. The method of any one Embodiments 322-330, wherein a nucleotide sequence associated with a condition, disorder or disease is hemizygous.

334. The method of any one Embodiments 322-333, wherein a nucleotide sequence associated with a condition, disorder or disease is in a genome.

335. The method of any one Embodiments 322-334, wherein a nucleotide sequence associated with a condition, disorder or disease is in a genome of some but not all cells.

336. The method of any one Embodiments 322-335, wherein a nucleotide sequence associated with a condition, disorder or disease is in a germline genome.

337. The method of any one Embodiments 322-336, wherein a nucleotide sequence associated with a condition, disorder or disease is a mutation.

338. The method of any one Embodiments 322-337, wherein a nucleotide sequence associated with a condition, disorder or disease is a G to A mutation.

339. The method of any one Embodiments 322-337, wherein a nucleotide sequence associated with a condition, disorder or disease is a G to A mutation in SERPINA1.

340. The method of any one Embodiments 322-337, wherein a nucleotide sequence associated with a condition, disorder or disease corresponds to 1024 G>A (E342K) mutation in human SERPINA1 gene.

341. The method of any one Embodiments 322-337, wherein a nucleotide sequence associated with a condition, disorder or disease is 1024 G>A (E342K) mutation in human SERPINA1 gene.

342. The method of any one Embodiments 322-337, wherein a first animal, embryo or cell is a NOD.Cg-Prkdcscid Il2rgtm1Wjl Tg(SERPINA1*E342K)#Slew/SzJ mouse (Jackson Laboratory Stock No: 028842).

343. A method for assessing an agent or a composition thereof for use in adenosine editing, comprising steps of:
administering an agent to an animal, embryo or cell of any one of the preceding Embodiments.

344. The method of Embodiment 343, wherein the agent or a composition thereof is an oligonucleotide composition.

345. The method of any one of Embodiments 343-344, wherein the agent or composition thereof provides a higher editing level in the engineered animal, embryo or cell (“engineered editing level”) compared to that in a reference animal, embryo or cell not engineered to express the ADAR1 polypeptide or a characteristic portion thereof (“reference editing level”).

346. A method, comprising expressing an RNA in an animal, embryo or cell of any one of the preceding Embodiments, wherein a target adenosine of the RNA is edited.

347. The method of Embodiment 346, comprising administering to the animal, embryo or cell an agent or a composition thereof targeting the target adenosine.

348. The method of Embodiment 346, comprising administering to the animal, embryo or cell an oligonucleotide or an oligonucleotide composition targeting the target adenosine.

349. The method of any one of Embodiments 346-348, wherein the targeted adenosine is edited at a higher level (“engineered editing level”) compared to that in a reference animal, embryo or cell not engineered to express the ADAR1 polypeptide or a characteristic portion thereof (“reference editing level”).

350. The method of any one of Embodiments 343-349, wherein the engineered editing level is about or at least about 1.5, 2, 3, 4, 5, 6, 7, 8, 9, 10, 15, 20, 30, 40, 50, 100 or more fold of the reference editing level.

351. The method of any one of Embodiments 343-349, wherein the engineered editing level is about or at least about 2 fold of the reference editing level.

352. The method of any one of Embodiments 343-349, wherein the engineered editing level is about or at least about 5 fold of the reference editing level.

353. The method of any one of Embodiments 343-349, wherein the engineered editing level is about or at least about 10 fold of the reference editing level.

354. A method for characterizing an ADAR1 polypeptide or a characteristic portion thereof, comprising:
expressing an ADAR1 polypeptide or a characteristic portion thereof in an engineered host cell of an animal or an animal;
assessing expression level of an ADAR1 polypeptide or a characteristic portion thereof, or editing level of a target adenosine in a transcript.

355. The method of Embodiment 354, wherein the ADAR1 polypeptide or a characteristic portion thereof is a human ADAR1 polypeptide or a characteristic portion thereof.

356. The method of any one of Embodiments 354-355, wherein the animal is a non-human animal.

357. The method of any one of Embodiments 354-355, wherein the animal is a rodent.

358. The method of any one of Embodiments 354-355, wherein the animal is a mouse.

359. The method of any one of Embodiments 354-355, wherein the animal is a rat.

360. The method of any one of Embodiments 354-359, comprising assessing expression level of the ADAR1 polypeptide or a characteristic portion thereof.

361. The method of any one of Embodiments 354-360, comprising assessing editing level of a target adenosine in a transcript.

362. The method of Embodiment 361, comprising administering to the host cell or animal an oligonucleotide composition which targets the target adenosine.

363. The method of Embodiment 362, wherein the oligonucleotide composition is of WV-38700, and the target adenosine is its targeted adenosine in a human or mouse UGP2 transcript.

364. The method of any one of Embodiments 362-363, wherein the oligonucleotide composition is of WV-38702, and the target adenosine is its targeted adenosine in a human or mouse UGP2 transcript.

365. The method of any one of Embodiments 362-364, wherein the oligonucleotide composition is of WV-40590, and the target adenosine is its targeted adenosine in a human or mouse UGP2 transcript.

366. The method of any one of Embodiments 362-365, wherein the oligonucleotide composition is of WV-40592, and the target adenosine is its targeted adenosine in a human or mouse SRSF1 transcript.

367. The method of any one of Embodiments 362-366, wherein the oligonucleotide composition is of WV-38697, and the target adenosine is its targeted adenosine in a human or mouse EEF1A1 transcript.

368. The method of any one of Embodiments 362-367, wherein the oligonucleotide composition is of WV-38699, and the target adenosine is its targeted adenosine in a human or mouse EEF1A1 transcript.

369. The method of any one of Embodiments 354-368, wherein the host cell is hepatocyte.

370. The method of any one of Embodiments 354-369, comprising comparing an expression level of an ADAR1 polypeptide or a characteristic portion thereof to that in a reference cell or animal not engineered to express an ADAR1 polypeptide or a characteristic portion thereof.

371. The method of any one of Embodiments 354-370, comprising comparing a target adenosine editing level to that in a reference cell or animal not engineered to express an ADAR1 polypeptide or a characteristic portion thereof.

372. A method for characterizing an agent or an oligonucleotide or a composition, comprising:
administering the agent or oligonucleotide or composition to a cell or a population thereof comprising or expressing an ADAR1 polypeptide or a characteristic portion thereof, or a polynucleotide encoding an ADAR1 polypeptide or a characteristic portion thereof.

373. The method of Embodiment 372, wherein the cell is a cell of any one of the preceding Embodiments.

374. A method for characterizing an oligonucleotide or a composition, comprising:
administering the oligonucleotide or composition to a non-human animal or a population thereof comprising or expressing an ADAR1 polypeptide or a characteristic portion thereof, or a polynucleotide encoding an ADAR1 polypeptide or a characteristic portion thereof.

375. The method of Embodiment 374, wherein the animal is an animal of any one of the preceding Embodiments.

376. The method of any one of Embodiments 372-375, wherein activity levels of an oligonucleotide or composition observed from a cell or a cell from an animal, or a population thereof, is more similar to those observed in a comparable human cell or a population thereof compared to those observed in a cell prior to engineering or a cell from an animal prior to engineering, or a population thereof.

377. The method of Embodiment 376, wherein a comparable human cell is of the same type as a cell or a cell from an animal.

378. The method of any one of Embodiments 372-377, wherein an agent or an oligonucleotide or a composition is assessed for preventing or treating a condition, disorder or disease, wherein the cell or animal is a cell or animal of any one of Embodiments 165-180.

379. The method of Embodiment 378, wherein a condition, disorder or disease is a condition, disorder or disease associated with a G to A mutation in human SERPINA1.

380. The method of Embodiment 378, wherein a condition, disorder or disease is a condition, disorder or disease associated with a 1024 G>A in human SERPINA1.

381. The method of Embodiment 378, wherein a condition, disorder or disease is alpha-1 antitrypsin deficiency.

382. A method, comprising:
assessing an agent or a composition thereof in a cell, tissue or animal, wherein the cell, tissue or animal is or comprises a cell, tissue or organ associated or of a condition, disorder or disease, and/or comprises a nucleotide sequence associated with a condition, disorder or disease; and
administering to a subject susceptible to or suffering from a condition, disorder or disease an effective amount of an agent or a composition for preventing or treating the condition, disorder or disease.

383. A method, comprising:
administering to a subject susceptible to or suffering from a condition, disorder or disease an effective amount of an agent or a composition for preventing or treating the condition, disorder or disease, wherein the agent or composition is assessed in a cell, tissue or animal, wherein the cell, tissue or animal is or comprises a cell, tissue or organ associated or of a condition, disorder or disease, and/or comprises a nucleotide sequence associated with a condition, disorder or disease.

384. The method of Embodiment 382 or 383, wherein the subject is a human.

385. The method of any one of Embodiments 382-383, wherein a condition, disorder or disease is associated with a G to A mutation.

386. The method of any one of Embodiments 382-383, wherein a condition, disorder or disease is associated with 1024 G>A (E342K) mutation in human SERPINA1 gene.

387. The method of any one of Embodiments 382-386, wherein a condition, disorder or disease is alpha-1 antitrypsin deficiency.

388. The method of any one of Embodiments 382-386, wherein the cell or animal is of any one of Embodiments 165-180.

389. A method, comprising
administering an agent or a composition thereof to a cell or a population thereof; and
assessing editing of an adenosine in a transcript in a cell or a population thereof.

390. The method of Embodiment 389, wherein the cell or a population thereof is grown in vitro.

391. The method of Embodiment 389, wherein the agent or composition thereof is administered to a cell or a population thereof in an animal.

392. A method, comprising
administering an agent or a composition thereof to an animal or a population thereof; and
assessing editing of an adenosine in a transcript in one or more cells or tissues of the animal or a population thereof.

393. The method of any one of Embodiments 389-392, wherein the agent is an oligonucleotide.

394. A method, comprising
administering an oligonucleotide composition to a cell or a population thereof, and
assessing editing of an adenosine in a transcript in a cell or a population thereof.

395. The method of Embodiment 394, wherein the cell or a population thereof is grown in vitro.

396. The method of Embodiment 394, wherein the oligonucleotide composition is administered to a cell or a population thereof in an animal.

397. A method, comprising
administering an oligonucleotide composition to an animal or a population thereof; and
assessing editing of an adenosine in a transcript in one or more cells or tissues of the animal or a population thereof.

398. The method of any one of Embodiments 394-397, wherein the oligonucleotide composition is a chirally controlled oligonucleotide composition.

399. The method of any one of Embodiments 389-398, wherein the cell, animal, or a population thereof is of any one of the preceding Embodiments.

400. The method of any one of Embodiments 382-399, wherein the cell or tissue is a rodent cell or tissue, or an animal is a rodent.

401. The method of Embodiment 400, wherein the rodent is rat.

402. The method of Embodiment 400, wherein the rodent is mouse.

403. The method of any one of Embodiments 382-402, wherein the cell, tissue, or animal comprises or expresses human ADAR1 or a fragment thereof.

404. The method of any one of Embodiments 382-402, wherein the cell, tissue, or animal comprises or expresses human ADAR1 or a characteristic portion thereof.

405. The method of any one of Embodiments 382-404, wherein the agent, a composition thereof or the oligonucleotide composition provides increased editing level of an adenosine (“engineered editing level”) compared to that observed in a reference cell or animal (“reference editing level”), wherein the reference animal is not engineered to express the ADAR1 polypeptide or a characteristic portion thereof.

406. The method of any one of Embodiments 382-399, wherein the agent, a composition thereof or the oligonucleotide composition provides increased editing level of an adenosine (“engineered editing level”) compared to that observed in a reference cell or animal (“reference editing level”), wherein the reference animal is not engineered to express a human ADAR1 polypeptide or a characteristic portion thereof.

407. The method of any one of Embodiments 382-406, wherein the cell is a type of cell in a liver.

408. The method of any one of Embodiments 382-406, wherein the cell is hepatocyte.

409. The animal of any one of the preceding Embodiments, wherein the engineered editing level is about or at least about 1.5, 2, 3, 4, 5, 6, 7, 8, 9, 10, 15, 20, 30, 40, 50, 100 or more fold of the reference editing level.

410. The animal of any one of the preceding Embodiments, wherein the engineered editing level is about or at least about 2 fold of the reference editing level.

411. The animal of any one of the preceding Embodiments, wherein the engineered editing level is about or at least about 5 fold of the reference editing level.

412. The animal of any one of the preceding Embodiments, wherein the engineered editing level is about or at least about 10 fold of the reference editing level.

413. The method of any one of Embodiments 382-412, comprising obtaining or manufacturing an agent, a composition thereof or an oligonucleotide composition for the administering.

414. The method of Embodiment 413, wherein the agent, a composition thereof or the oligonucleotide composition is not manufactured for administration to a subject.

415. The method of Embodiment 413 or 414, wherein the agent, a composition thereof or the oligonucleotide composition is not manufactured as a drug product.

416. The method of any one of Embodiments 413-415, wherein the agent, a composition thereof or the oligonucleotide composition is not a commercial batch.

417. The method of Embodiment 413, wherein the agent, a composition thereof or the oligonucleotide composition is manufactured for administration to a subject.

418. The method of Embodiment 413 or 417, wherein the agent, a composition thereof or the oligonucleotide composition is manufactured as a drug product.

419. The method of any one of Embodiments 413 and 417-418, wherein the agent, a composition thereof or the oligonucleotide composition is a commercial batch.

420. The method of any one of Embodiments 382-419, comprising obtaining a batch of an agent, a composition thereof or an oligonucleotide composition after assessing.

421. The method of any one of Embodiments 382-420, comprising manufacturing a batch of an agent, a composition thereof or an oligonucleotide composition after assessing.

422. The method of any one of Embodiments 382-421, wherein the batch is a commercial batch.

423. The method of any one of Embodiments 382-422, comprising producing a drug product of an agent, a composition thereof or an oligonucleotide composition suitable for administration to a subject.

424. The method of any one of Embodiments 414-423, wherein a subject is a human.

425. The method of any one of Embodiments 382-424, comprising:
administering a sample from a commercial batch or a drug product of an agent, a composition thereof or an oligonucleotide composition a cell or animal, or a population thereof,
assessing editing an adenosine in a transcript in the cell, or one or more cells or tissues of the animal, or a population thereof.

426. The method of Embodiment 425, wherein editing of the adenosine is at a level comparable to that of a non-commercial production prior to the first commercial production.

427. The method of any one of Embodiments 425-426, wherein editing of the adenosine is at a level comparable to that of another batch of commercial production or drug product.

428. The method of any one of Embodiments 425-427, wherein editing of the adenosine is at a level comparable to that of a reference sample or drug product.

429. The method of any one of Embodiments 425-428, wherein the editing of the adenosine is at a level that is within a reference range.

430. The method of any one of Embodiments 425-429, comprising releasing the commercial batch or drug product for delivery, distribution or administration.

431. The method of Embodiment 425, wherein editing of the adenosine is at a level not comparable to that of a non-commercial production prior to the first commercial production.

432. The method of Embodiment 425 or 431, wherein editing of the adenosine is at a level not comparable to that of another batch of commercial production or drug product.

433. The method of any one of Embodiments 425 and 431-432, wherein editing of the adenosine is at a level not comparable to that of a reference sample or drug product.

434. The method of any one of Embodiments 425 and 431-433, wherein the editing of the adenosine is at a level that is not within a reference range.

435. The method of any one of Embodiments 425 and 431-434, comprising rejecting the commercial batch or drug product for delivery, distribution or administration.

### EXAMPLES

The following examples are provided so as to describe to the skilled artisan how to make and use cell, tissues, animals, methods, compositions, etc. described herein; and are not intended to limit the scope of the present disclosure. Unless indicated otherwise, temperature is indicated in Celsius and pressure is at or near atmospheric.

Certain examples of provided technologies (compounds (oligonucleotides, reagents, etc.), compositions, methods (methods of preparation, use, assessment, etc.), polynucleotides, polypeptides (e.g., ADAR1 polypeptides or characteristic portions thereof), cells, tissues, non-human animals, etc.) were presented herein.

Those skilled in the art appreciates that many technologies can be utilized to assess properties and/or activities of provided technologies, e.g., those described in Examples below.

Various technologies for preparing oligonucleotides and oligonucleotide compositions (both stereorandom and chirally controlled) are known and can be utilized in accordance with the present disclosure, including, for example, methods and reagents described in US 9982257, US 20170037399, US 20180216108, US 20180216107, US 9598458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, and/or WO 2019/032612.

### Example 1 - Generation of Targeting Vectors Comprising a Human Adenosine Deaminase Acting on RNA 1 (ADARI) Coding Sequence.

A human ADAR-1 coding isoform polynucleotide is made using standard molecular biology techniques recognized in the art (e.g., A DNA fragment is created through de novo DNA synthesis, or cloning). The fragment contains a complete coding sequence of human Adenosine Deaminase Acting on RNA 1 or functional and characteristic portions thereof (as represented by SEQ ID NOs: 27-40).

A human ADAR-1 isoform p110 (e.g., transcript variant 4) polynucleotide was synthesized and confirmed with Sanger sequencing. The fragment (2942 bp) was digested with PacI and NheI and ligated into the mROSA-KI-12p vector (linearized with PacI and NheI) using methods known in the art. The fragment contained the complete coding sequence of a human Adenosine Deaminase Acting on RNA 1 transcript (SEQ ID NO: 14) encoding the p110 polypeptide (SEQ ID NO: 16).

A human ADAR-1 isoform p150 (transcript variant 1) polynucleotide was also synthesized and confirmed with Sanger sequencing. The fragment (3827 bp) was digested with PacI and NheI and ligated into the mROSA-KI-12p vector (linearized with PacI and NheI) using methods known in the art. The fragment contained a complete coding sequence of a human Adenosine Deaminase Acting on RNA 1 transcript (SEQ ID NO: 3) encoding the p150 polypeptide (SEQ ID NO: 6).

The ADAR1 p110 and p150 encoding polynucleotides described in the paragraphs above were ligated into the mROSA-KI-12p targeting vector to produce Targeting Vector A (see FIG. 1 and FIG. 4) and Targeting Vector B (see FIG. 2). To aid with propagation, the plasmids containing the Targeting Vectors contained an ampicillin resistance positive selection marker and an origin site for replication. The Targeting Vectors contain in a 5′ to 3′ order: a 5′ (aka left) homology arm (SEQ ID NO: 62), an adenovirus derived splice acceptor (SEQ ID NO: 57), a KOZAK sequence, the coding sequence of interest (e.g., a CDS for human ADAR1) (SEQ ID NO: 14 or SEQ ID NO: 3), a Woodchuck Hepatitis Virus Posttranscriptional Regulatory Element (WPRE) (SEQ ID NO: 56), a bovine growth hormone (bGH) polyadenylation signal (SEQ ID NO: 59), and a 3′ (aka right) homology arm (SEQ ID NO: 63). For Targeting Vector A and B the homology arms targeting the ROSA26 loci were identical, and are homologous to the region surrounding the first exon of the mouse ROSA26 allele. The 5′ homology arm encompasses 1812 bp and is represented by SEQ ID NO: 62. The 3′ homology arm encompasses 1779 bp and is represented by SEQ ID NO: 63. For Targeting Vector A, the complete targeting sequence is represented by SEQ ID NO: 64. For Targeting Vector B, the complete targeting sequence is represented by SEQ ID NO: 65.

Those skilled in the art will recognize that polynucleotides encoding ADAR1 polypeptides or characteristic portions thereof may be inserted at various other locations in accordance with the present disclosure, e.g., other rodent transcriptional harbors (aka transcriptional hotspots, e.g., as described herein), endogenous mouse ADAR1 locus, and/or at different sites within an ROSA26 allele.

Following cloning of Targeting Vectors, restriction enzyme digestion and fragment size analysis can be used to confirm insertion of the CDS into the respective target vector. Those skilled in the art will recognize that alternative screening methodologies (e.g., Sanger sequencing, selective PCR, etc.) may be utilized to confirm construct creation.

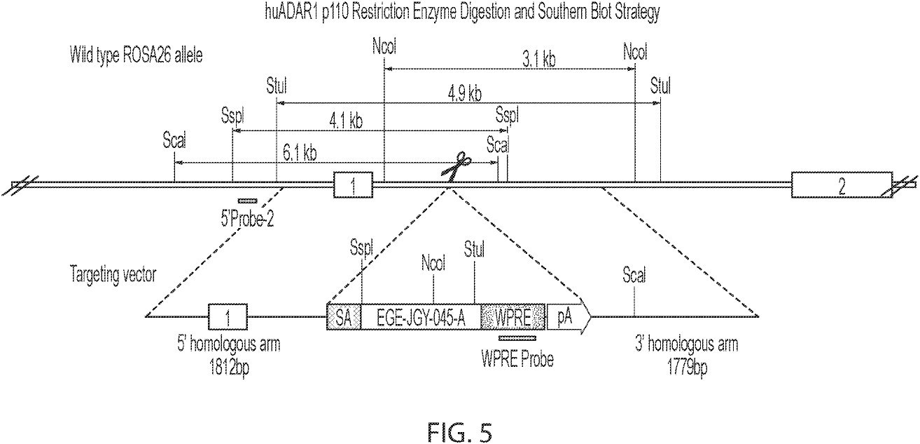

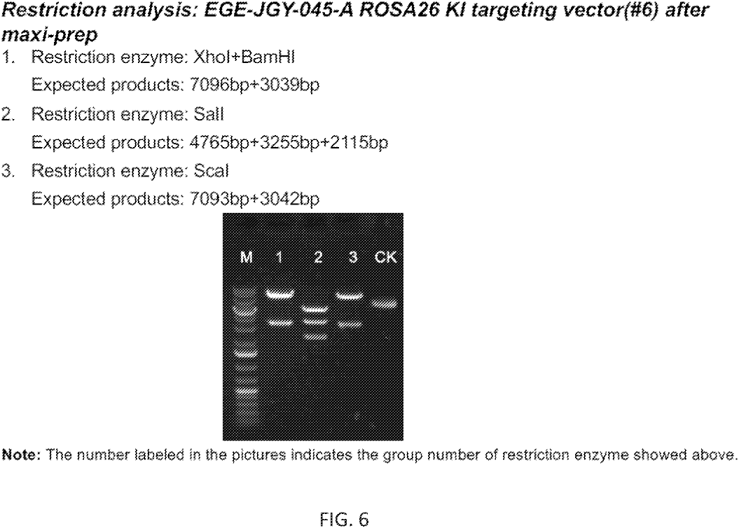

Restriction digestion of Targeting Vector A produced variable fragment sizes dependent upon restrictions enzymes employed (see FIG. 5). Three combinations of restriction enzymes were utilized to produce variable fragment sizes, with correct insertion the following products are observed: 1) XhoI + BamHI digestion produced linear fragments 7096 bp + 3039 bp in length; 2) SalI digestion produced linear fragments 4765 bp +3255 bp +2115 bp in length; 3) ScaI digestion produced linear fragments 7093 bp +3042 bp in length (see FIG. 6). Following digestion, clone sequences #6 and #7 displayed correct banding patterns, and following Sanger Sequencing, clone #6 was selected for further use.

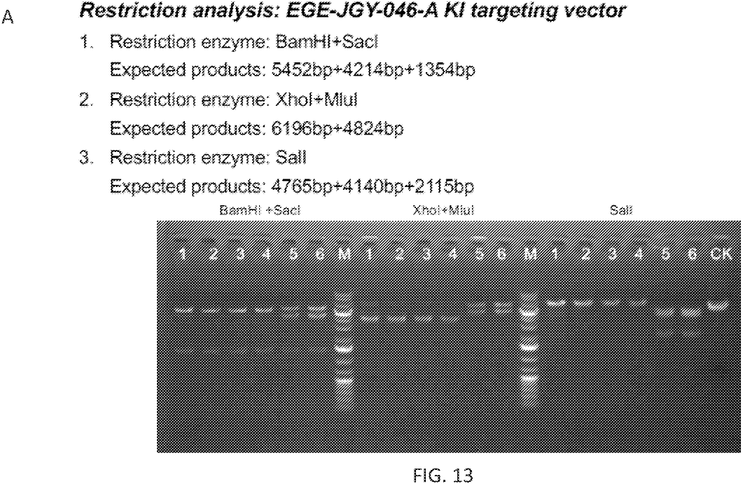

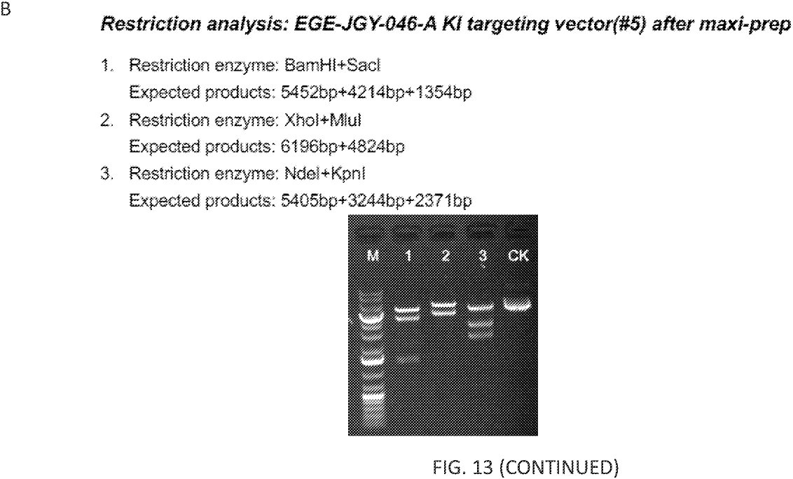

Restriction digestion of Targeting Vector B produced variable fragment sizes dependent upon restrictions enzymes employed (see FIG. 12). Three combinations of restriction enzymes were utilized to produce variable fragment sizes, with correct insertion the following products are observed: 1) BamHI + SacI digestion produced linear fragments 5452 bp + 4214 bp +1354 bp in length; 2) XhoI +MluI digestion produced linear fragments 6196 bp +4824 bp in length; 3) SalI digestion produced linear fragments 4765 bp + 4140 bp + 2115 bp in length; 4) NdeI + KpnI digestion produced linear fragments 5405 bp + 3244 bp + 2371 bp in length (see FIG. 13). Following digestion, clone sequences #5 and #6 displayed correct banding patterns, and following Sanger Sequencing, clone #5 was selected for further use.

Those skilled in the art appreciate that other technologies, e.g., vectors, restriction enzymes, etc., may also be utilized in accordance with the present disclosure to prepare polynucleotides for introduction into non-human animals and cells thereof.

### Example 2 - Generation of Mouse Zygotes and Animals Comprising a Human ADAR1 Coding Sequence.

Various technologies can be utilized in accordance with the present disclosure. For example, in some embodiments, Targeting Vectors, (e.g., as described herein), are injected into mouse zygotes in combination with a site directed enzyme capable of generating site specific double strand breaks and/or single strand nicks (e.g., CRISPR/Cas9, TALENs, and/or Zinc Finger Nucleases). Those skilled in the art will recognize that there are myriad mouse genotypes amenable to zygotic injection and transgenic animal creation. Following injection, zygotes are transferred into surrogate mothers. After gestation, pups are genotyped using methods known in the art (e.g., tail clipping) and animals with potential insertions are noted for further analysis.

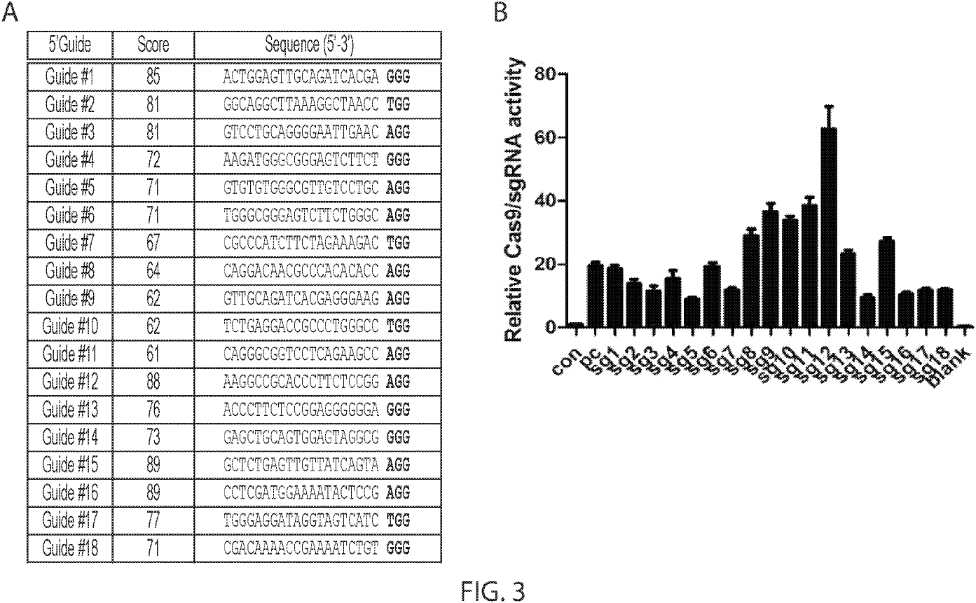

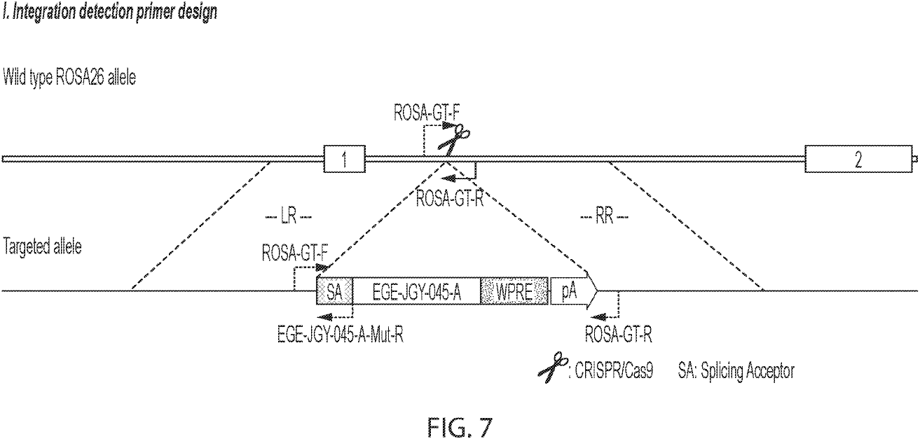

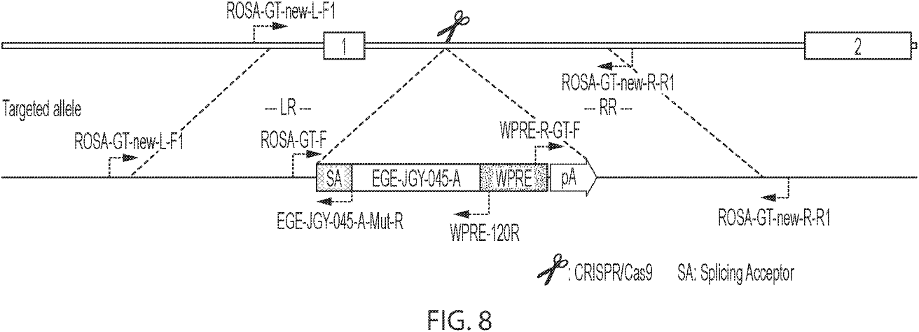

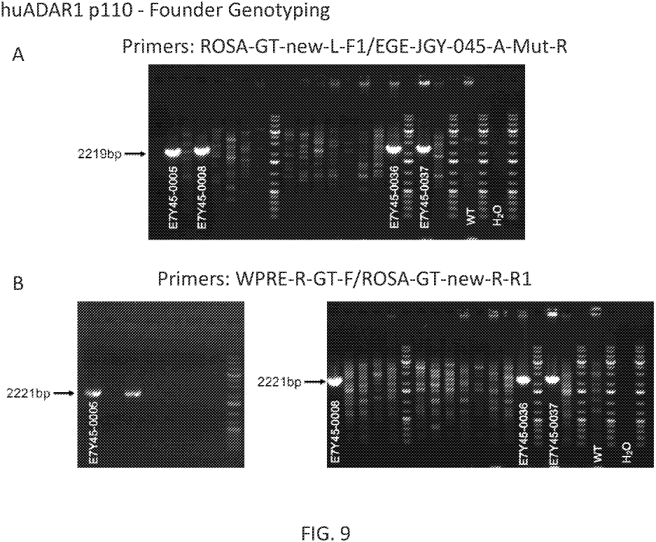

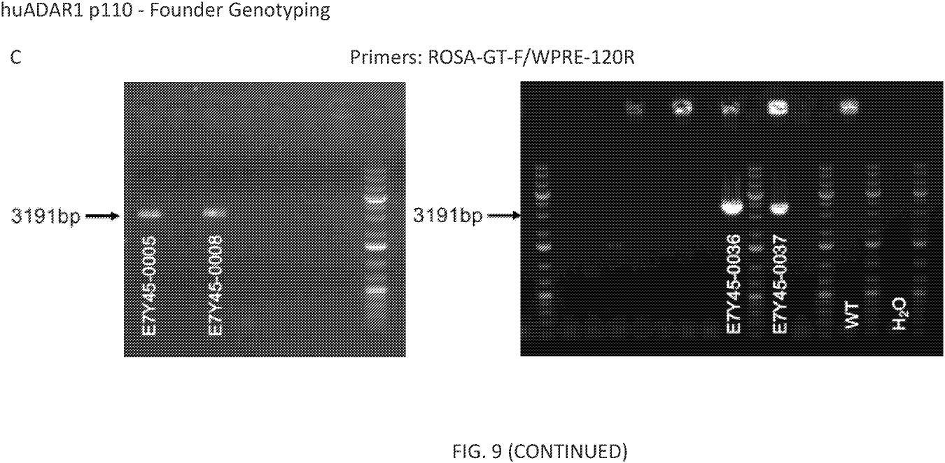

An injection mix comprising the Extreme Genome Editing (EGE®) System reagents (developed by and commercially available from Biocytogen, comprising sgRNA 12 identified in FIG. 3) in combination with Targeting Vector A (e.g., as described in Example 1), was injected into C57BL/6J mouse zygotes to create transgenic animals. Zygotes were injected utilizing techniques known in the art; 324 injected zygotes were then transferred into surrogate mothers. Of these 324 zygotes, 38 generated viable progeny. Pups were genotyped using PCR (Primer combination: 6, 7, and 8; see Table 2 and FIGS. 7 and 8) and subsequent Sanger Sequencing. Founder pups #E7Y45-0005, #E7Y45-0008, #E7Y45-0036 and #E7Y45-0037 were confirmed to contain the CDS of interest (e.g., encoding ADAR1 p110) genomically inserted through Homologous Recombination (see FIG. 9).

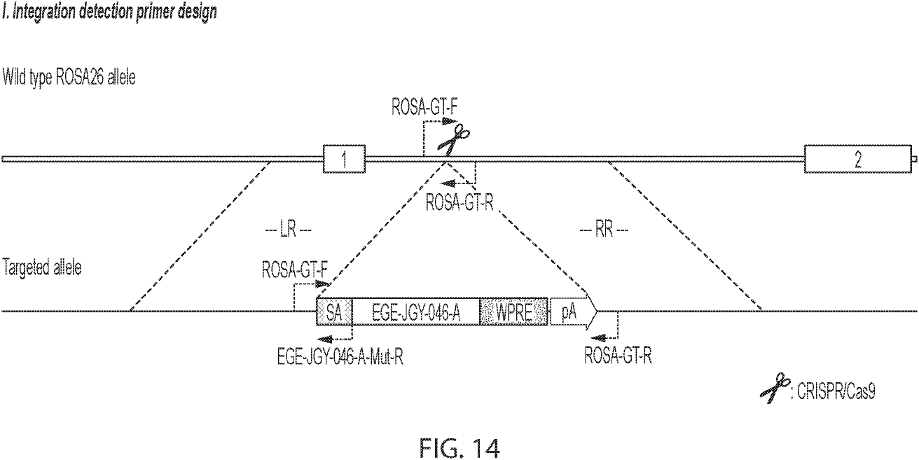

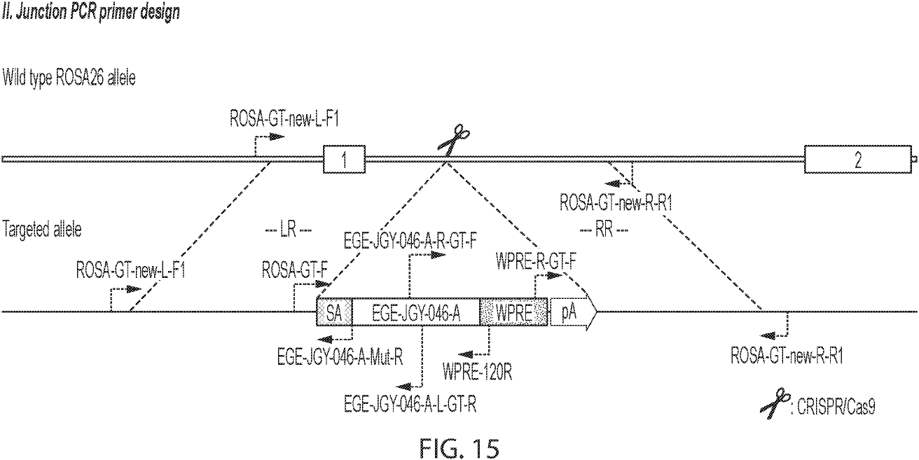

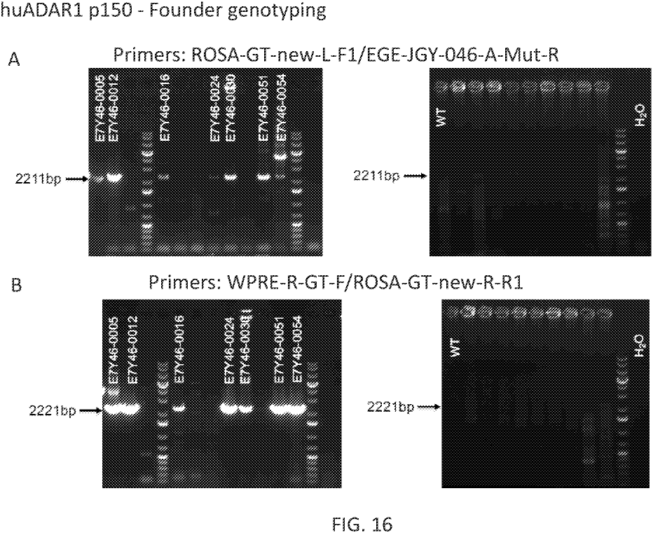

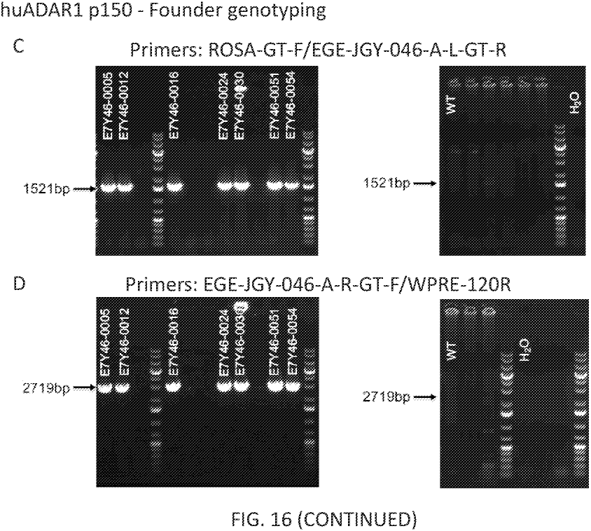

An injection mix comprising the Extreme Genome Editing (EGE®) System reagents (developed by and commercially available from Biocytogen, comprising sgRNA 12 identified in FIG. 3) in combination with Targeting Vector B (e.g., as described in Example 1), was injected into C57BL/6J mouse zygotes to create transgenic animals. Zygotes were injected utilizing techniques known in the art; 462 injected zygotes were transferred into surrogate mothers. Of these 462 zygotes, 71 generated viable progeny. Pups were genotyped using PCR (Primer combination: 6, 8, 10, and 11; see Table 2 and FIGS. 14 and 15) and subsequent Sanger Sequencing. Founder pups #E7Y46-0012 and #E7Y46-0051 were confirmed to contain the CDS of interest (e.g., encoding ADAR1 p150) genomically inserted through Homologous Recombination (see FIG. 16).

TABLE 2

Primers Used for Genotyping Transgenic Mice

| | | | |
|---|---|---|---|
| Name | Forward Primer | Reverse Primer | Predicted Product Size WT:MUT |
| Combination 1: mROSA26-MSD-F + mROSA26-MSD-R | CGTGTTCGTGCAAG TTGAGTCCATC (SEQ ID NO: 66) | TTTGATAAGGCTGC AGAAGGAGCGG (SEQ ID NO: 67) | 938 bp : N/A |
| Combination 2: mROSA-5′Probe-F2 + mROSA-5′Probe-R2 | CCTCCTAGCGACCT AAACCATTAT (SEQ ID NO: 68) | CCTGGTTTTAGTAG GTAATCGCTCA (SEQ ID NO: 69) | 551 bp : N/A |
| Combination 3: WPRE-Probe-F + WPRE-Probe-R | GTGGATACGCTGCT TTAATGCC (SEQ ID NO: 70) | AAGGGAGATCCGA CTCGTCTG (SEQ ID NO: 71) | N/A : 500 bp |
| Combination 4: ROSA-GT-F + | AGTCGCTCTGAGTT GTTATCAG | TGAGCATGTCTTTA ATCTACCTCGATG | 469 bp : 4306 bp |
| ROSA-GT-R | (SEQ ID NO: 72) | (SEQ ID NO: 73) | |
| Combination 5: ROSA-GT-F + EGE-JGY-045-A-Mut-R | AGTCGCTCTGAGTT GTTATCAG (SEQ ID NO: 72) | CAGGGCAGAGGAG TCAGACACATTGA (SEQ ID NO: 74) | N/A : 264 bp |
| Combination 6: ROSA-GT-new-L-F1 + EGE-JGY-045-A-Mut-R | GGCGTTCAGGAAG ATTATGGAGGGG (SEQ ID NO: 75) | CAGGGCAGAGGAG TCAGACACATTGA (SEQ ID NO: 74) | N/A : 2219 bp |
| Combination 7: ROSA-GT-F + WPRE-120R | AGTCGCTCTGAGTT GTTATCAG (SEQ ID NO: 72) | ACGGGAAGCAATA GCATGATAC (SEQ ID NO: 76) | N/A : 3191 bp |
| Combination 8: WPRE-R-GT-F + ROSA-GT-new-R-R1 | ACGAGTCGGATCTC CCTTTGG (SEQ ID NO: 77) | TGGCTGCAGACTTA GCTTTCAGCTT (SEQ ID NO: 78) | N/A : 2221 bp |
| Combination 9: ROSA-GT-new-L-F1 + EGE-JGY-046-A-Mut-R | GGCGTTCAGGAAG ATTATGGAGGGG (SEQ ID NO: 75) | CTTGAAATGGATG GGTGTAGTATCC (SEQ ID NO: 79) | N/A : 2211 bp |
| Combination 10: ROSA-GT-F + EGE-JGY-046-A-L-GT-R | AGTCGCTCTGAGTT GTTATCAG (SEQ ID NO: 72) | GCCATTGTAATGAA CAGGTGGTTTCAG (SEQ ID NO: 80) | N/A : 1521 bp |
| Combination 11: EGE-JGY-046-A-R-GT-F + WPRE-120R | ACGCAGAGTTCCTC ACCTGTAATAT (SEQ ID NO: 81) | ACGGGAAGCAATA GCATGATAC (SEQ ID NO: 76) | N/A : 2719 bp |

### Example 3 - Generation of Stable Germline Integrated Mice Comprising a Human ADAR1 Coding Sequence

Founder pups created as described in Example 2 above were used to establish stable germline transmission of huADAR1 using standard methods known in the art.

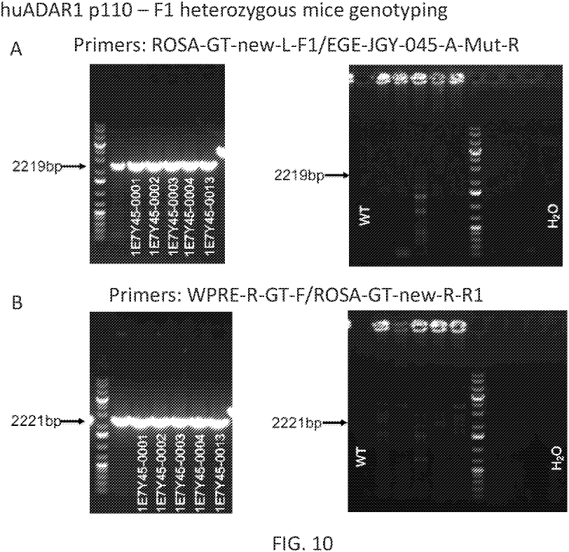

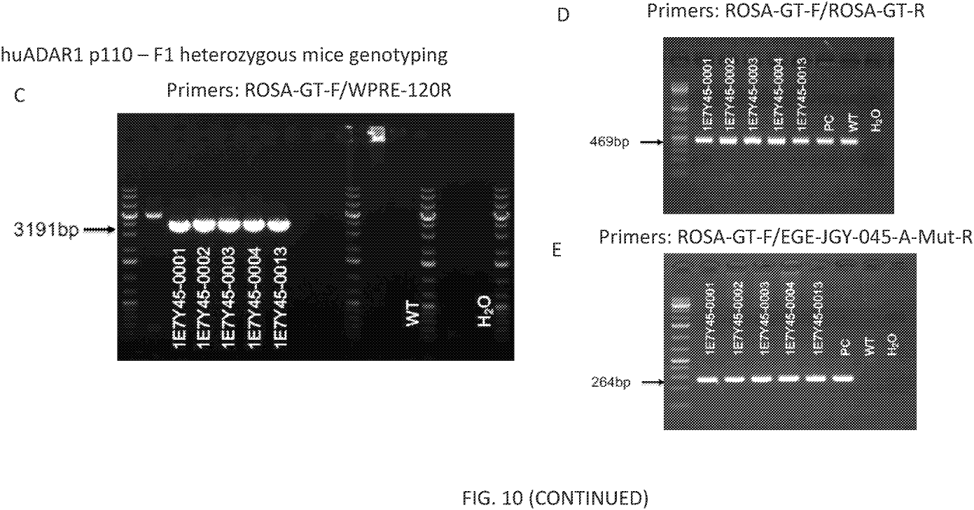

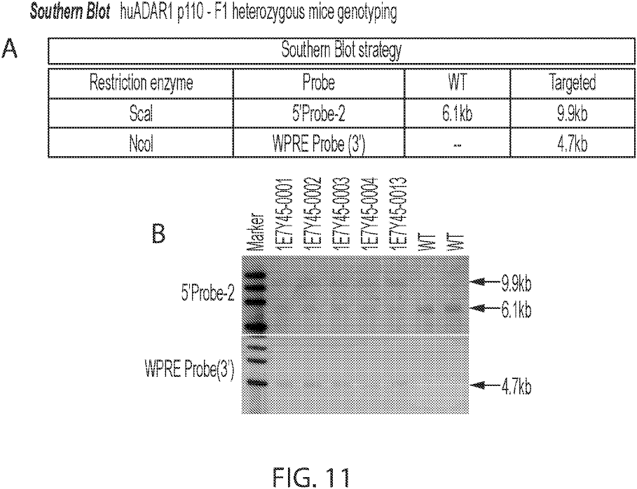

Founder pups heterozygous for huADAR1 p110 insertions were crossed with C57BL/6J mice to produce stable F1 progeny. In one cross, a WT male C57BL/6J animal was mated with confirmed allele carrier female #E7Y45-0005, the resultant pups were genotyped using PCR (Primer combination: 4, 5, 6, 7, and 8; see Table 2, and FIGS. 7 and 8) and 4 out of 7 pups genotyped were confirmed to be heterozygous for the huADAR1 p110 insertion (see FIG. 10). In another cross, a number of WT female C57BL/6J were mated with a confirmed allele carrier male #E7Y45-0037, the resultant pups were genotyped using PCR (Primer combination: 4, 5, 6, 7, and 8; see Table, and FIGS. 7 and 8) and 1 out of 54 pups genotyped were confirmed to be heterozygous for the huADAR1 p110 insertion (see FIG. 10). These F1 animals were additionally subject to southern blot genotyping using methods known in the art to confirm huADAR1 p110 transgene presence (see FIG. 5 and Table 3 for predicted band sizes and FIG. 11 for results). The five F1 animals predicted to be heterozygous for huADARI p110 were confirmed to be positive carriers of the allele. These results show stable germline transmission of the huADAR1 p110 transgene.

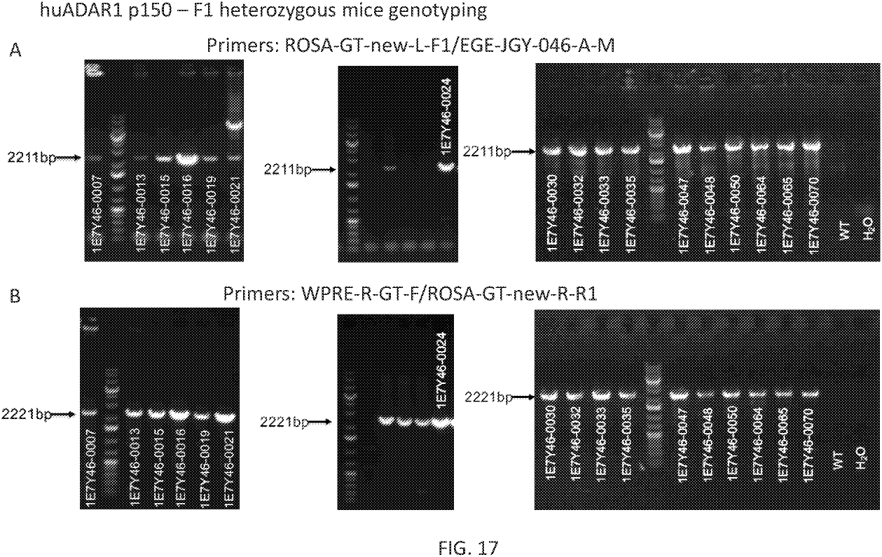

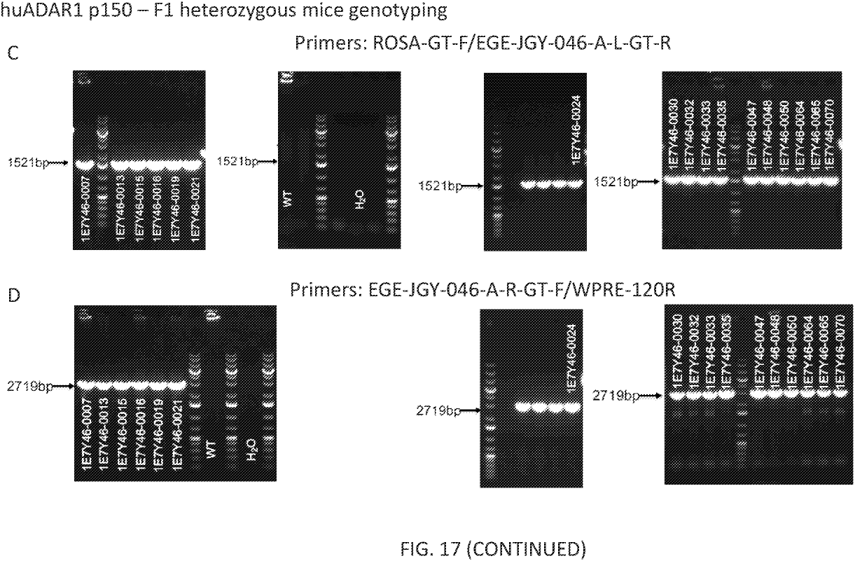

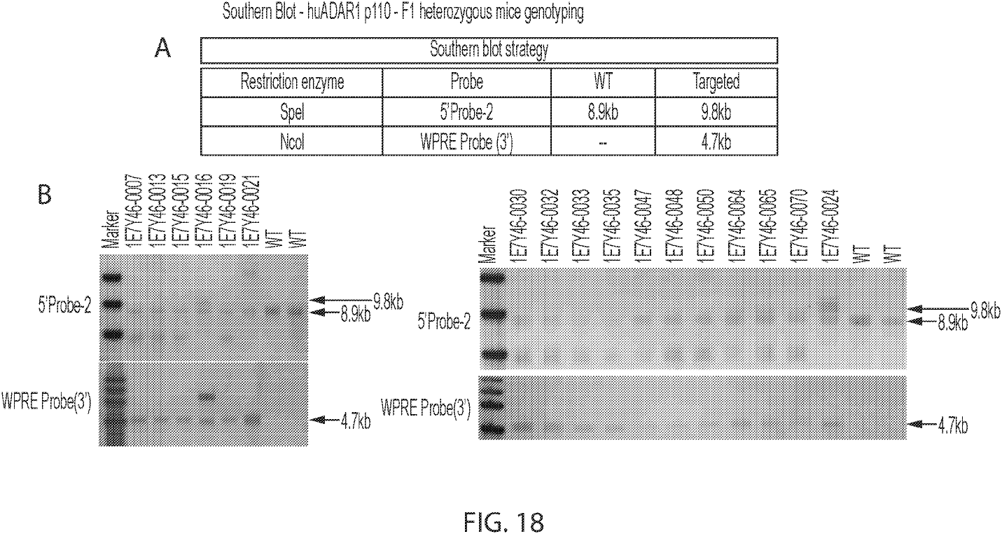

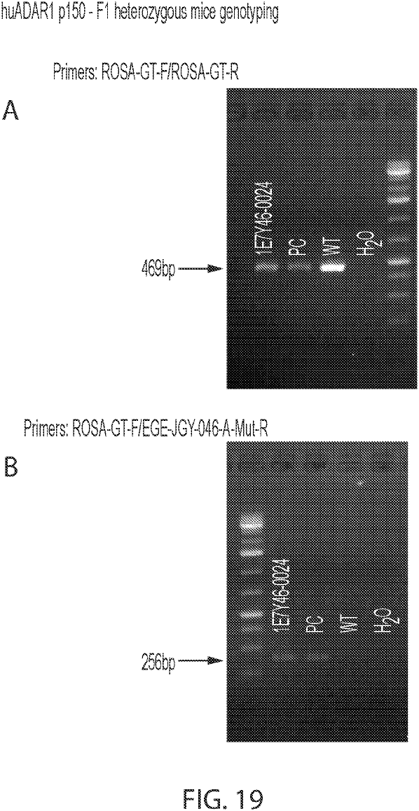

Founder pups heterozygous for huADAR1 p 150 insertions were crossed with C57BL/6J mice to produce stable F1 progeny. In one cross, a WT female C57BL/6J animal was mated with confirmed allele carrier male #E7Y 46-0051, the resultant pups were genotyped using PCR (Primer combination: 8, 9, 10, and 11; see Table 2, genotyping data not shown), all of the first generation resultant pups genotyped failed to be carriers for the huADAR1 p 150 insertion, a result that is not unexpected as germline transmission is not guaranteed from every founder animal for every cross. Additional crosses between WT female C57BL/6J animals and confirmed allele carrier male #E7Y46-0051 were performed and dozens of additional pups were produced. The resultant pups were genotyped using PCR (Primer combination: 8, 9, 10, and 11; see Table 2, FIG. 17) and 16 pups genotyped were positively identified as potential huADAR1 p150 insertion carriers (see FIG. 16). These F1 animals were additionally subject to southern blot genotyping using methods known in the art to confirm huADAR1 p150 transgene presence (see FIG. 12 and Table 3 for predicted band size and FIG. 17 for results). Of these 16 animals, 1 animal was confirmed to be a huADAR1 p150 heterozygous carrier through Southern blot analysis followed by additional PCR analysis (see FIG. 18). These results show stable germline transmission of a huADAR1 p150 transgene.

TABLE 3

Restriction Enzyme Strategy for Southern Blot Genotyping Transgenic Mice

| | | | | |
|---|---|---|---|---|
| Target Vector | Restriction Enzyme | Probe | WT fragment size | Insertion fragment size |
| A (ADAR1p110) | ScaI | 5′Probe-2 | 6.1 kb | 9.9 kb |
| A (ADAR1p110) | NcoI | WPRE Probe (3′) | - | 4.7 kb |
| B (ADAR1p150) | SpeI | 5′Probe-2 | 8.9 kb | 9.8 kb |
| B (ADAR1p150) | NcoI | WPRE Probe (3′) | - | 4.7 kb |

Homozygous engineered animals can be created through standard breeding techniques in accordance with the present disclosure. For example, heterozygous animals comprising a polynucleotide whose sequence encodes human ADAR1 p110 were mated to produce multiple litters of pups, initially available genotyping data indicated 19 WT, 34 heterozygotes, and 13 homozygotes.

### Example 4 - In Vivo Expression of huADAR1 P110 in Transgenic Mice

As described in Example 3, stable huADAR1 expression was established for both huADAR1 p110 and huADAR1 p150 respectively. Animals were then subject to further analysis for confirmation of huADAR1 expression.

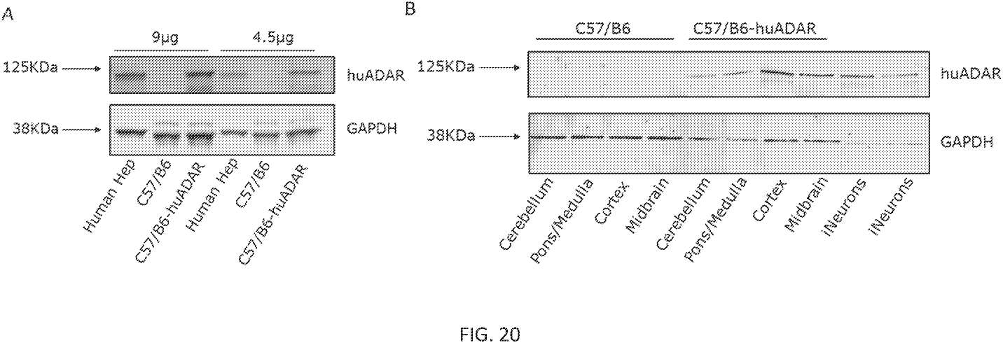

Heterozygous huADAR1 p110 animals and WT C57BL/6J mice were subject to euthanasia and tissue harvesting using methods known in the art. Protein samples were purified using standard methods known in the art. In parallel, primary human hepatocytes (Gibco). were propagated and harvested for protein samples. Following purification and concentration normalization, protein samples were analyzed using Western blot with GAPDH as the loading control (see FIG. 20A). Robust huADAR1 p110 expression comparable to levels observed in human hepatocytes was observed in huADAR1 p110 heterozygotic transgenic mice, while control C57BL/6J mice show no huADAR1 expression. These results revealed that huADAR1 p110 is robustly transcribed and translated and is stably expressed in heterozygotic transgenic animals.

Heterozygous huADAR1 p110 animals and WT C57BL/6J mice were subject to euthanasia and CNS tissue harvesting using methods known in the art. Protein samples were purified using standard methods known in the art. In parallel, induced Neurons (iNeurons) (commercially available from BrainXell) were propagated and harvested for protein samples. Following purification and concentration normalization, protein samples were analyzed using Western blot with GAPDH as the loading control (see FIG. 20B). Robust huADAR1 p110 expression comparable to levels observed in human iNeurons was observed in the Cerebellum, Pons/Medulla, Cortex, and Midbrain of huADAR1 p110 heterozygotic transgenic mice. These results revealed that huADAR1 p110 is robustly transcribed and translated and is stably expressed in heterozygotic transgenic animals.

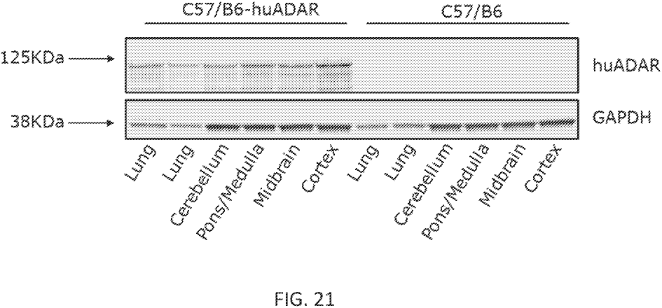

Heterozygous huADAR1 p110 animals and WT C57BL/6J mice were subject to euthanasia and lung tissue harvesting using methods known in the art. Protein samples were purified using standard methods known in the art. Following purification and concentration normalization, protein samples were analyzed using Western blot with GAPDH as the loading control (see FIG. 21). Robust huADAR1 p110 expression was observed in huADAR1 p110 heterozygotic transgenic mice lung samples when compared to control C57BL/6J mice which show no lung specific huADAR1 expression. These results revealed that huADAR1 p110 is robustly transcribed and translated and is stably expressed in heterozygotic transgenic animals.

Heterozygous and/or Homozygous huADAR1 p150 animals and WT C57BL/6J mice are subject to euthanasia and tissue harvesting using methods known in the art. Protein samples are purified using standard methods known in the art. In parallel, induced Neurons (iNeurons) (commercially available from BrainXell) are propagated and harvested for protein samples. Following purification and concentration normalization, protein samples are analyzed using Western blot with a suitable loading control (e.g., GAPDH). HuADAR1 p150 expression is assessed.

### Example 5 - In Vivo Editing of UGP2 in huADAR Transgenic Mice

Among other things, provided technologies, e.g., non-human animals engineered to comprise or express ADAR1 polypeptide or a characteristic portion thereof, are particularly useful for assessing agents for adenosine editing.

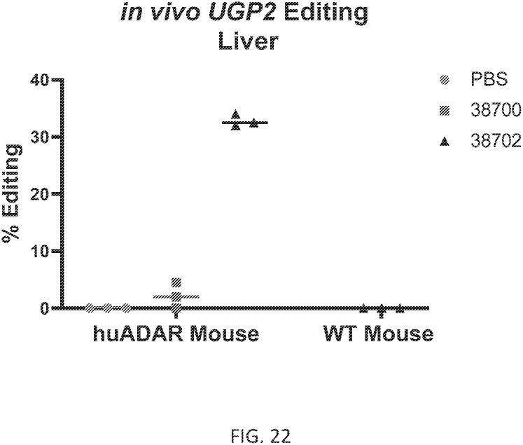

In some embodiments, oligonucleotide compositions (see Table 1) targeting UDP-glucose pyrophosphorylase 2 (UGP2) or control vehicle were introduced to huADAR1 p110 mice and control WT mice through a three dose schedule (e.g., day 0, day 2, and day 4) at 10 mg/kg oligonucleotide in PBS delivered subcutaneously, on day 6 mice were sacrificed for immediate biopsy. Liver samples were harvested from huADAR p110 animals and control animals; total RNA was then collected from the livers. As those skilled in the art appreciate, various technologies may be utilized in accordance with the present disclosure, e.g., the SV 96 Total RNA Isolation System Protocol (Promega: Z3505). RT-PCR using High-Capacity cDNA Reverse Transcription Kit with random hexamer primers (Life Technologies, Cat. No. 4388950) was used to generate a cDNA library. Primers flanking the edit site of the endogenously expressed target RNA were used (see Table 4) to amplify cDNA species of interest using the Phusion High-Fidelity DNA Polymerase protocol (Thermo: F-530XL). The PCR product was Sanger-sequenced and the percent of ADAR mediated editing was calculated using the program EditR. In vivo editing levels of UGP2 was higher in huADAR p110 animals when compared to WT animals (see FIG. 22).

TABLE 4

Exemplary Primers for Quantifying Site-Specific Oligonucleotide Mediated RNA Editing Levels

| | | |
|---|---|---|
| Name | Forward Primer | Reverse Primer |
| Combination 1: Mouse UGP2_FW + Mouse UGP2 Rev | CCTCCAGGGCATGGAGATAT C (SEQ ID NO: 82) | CCATGTCAATGGCATTCTGTT CC (SEQ ID NO: 83) |
| Combination 2: Human UGP2_FW + Human UGP2 Rev | ACAGAAGCTTGGTACCCTCC (SEQ ID NO: 84) | TGTGCTTTTGGCACTTGAGC (SEQ ID NO: 85) |
| Combination 3: Mouse EEF1A 1_FW + Mouse EEF1A1 Rev | CACCGAGCCACCATACAGTC (SEQ ID NO: 86) | GAACACCAGTCTCCACTCGG (SEQ ID NO: 87) |
| Combination 4: Human EEF1A1_FW + Human EEF1A1 Rev | GGTGTTGGTGAATTTGAAGC TGG (SEQ ID NO: 88) | GTCAGTTGGACGAGTTGGTG G (SEQ ID NO: 89) |
| Combination 5: Mouse SRSF1 _FW + Mouse SRSF1 Rev | CTACATTCCCCTCATGTCTTT GG (SEQ ID NO: 90) | GATCTGATCCTCCTCAATCCT CC (SEQ ID NO: 91) |
| Combination 6: Human SRSF1 _FW + Human SRSF1 Rev | TCCTAACATCTACATTCCCTT CG (SEQ ID NO: 92) | CTAAGCACTGTGACAAATTA GCC (SEQ ID NO: 93) |

### Example 6 - In Vivo Editing of EEF1A1 in huADAR Transgenic Mice

Among other things, provided technologies, e.g., non-human animals engineered to comprise or express ADAR1 polypeptide or a characteristic portion thereof, are particularly useful for assessing agents for adenosine editing.

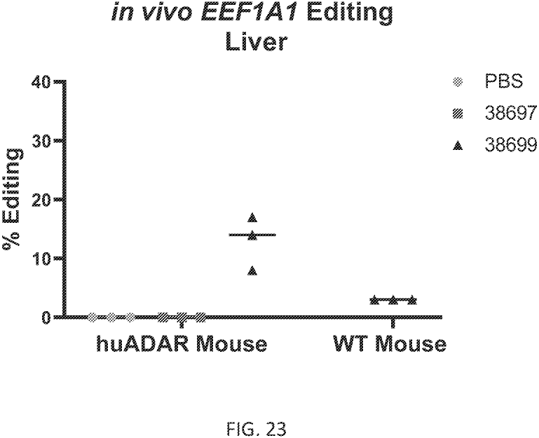

In some embodiments, oligonucleotide compositions (see Table 1) targeting eukaryotic translation elongation factor 1 alpha 1 (EEF1A1) or control vehicle were introduced to huADAR1 p110 mice and control WT mice through a three dose schedule (e.g., day 0, day 2, and day 4) at 10 mg/kg oligonucleotide in PBS delivered subcutaneously, on day 6 mice were sacrificed for immediate biopsy. Liver samples were harvested from huADAR p110 animals and control animals; total RNA was then collected from the livers. As those skilled in the art appreciate, various technologies may be utilized in accordance with the present disclosure, e.g., the SV 96 Total RNA Isolation System Protocol (Promega: Z3505). RT-PCR using High-Capacity cDNA Reverse Transcription Kit with random hexamer primers (Life Technologies, Cat. No. 4388950) was used to generate a cDNA library. Primers flanking the edit site of the endogenously expressed target RNA were used (see Table 4) to amplify cDNA species of interest using the Phusion High-Fidelity DNA Polymerase protocol (Thermo: F-530XL). The PCR product was Sanger-sequenced and the percent of ADAR mediated editing was calculated using the program EditR. In vivo editing levels of EEF1A1 was higher in huADAR p110 animals when compared to WT animals (see FIG. 23).

### Example 7 - In Vitro Editing of UGP2 and EEF1A1 in Primary Hepatocytes Derived from huADAR Transgenic Mice through Gymnotic and/or GalNAc-Mediated Oligonucleotide Uptake.

Among other things, provided technologies, e.g., non-human animals, cells, and tissues engineered to comprise or express ADAR1 polypeptide or a characteristic portion thereof, are particularly useful for assessing agents for adenosine editing. In certain embodiments, primary cells were harvested from huADAR1 transgenic animals and utilized for assessing RNA editing.

In some embodiments, oligonucleotide compositions were assessed in primary mouse hepatocyte cells derived from huADAR1 p110 animals. Concurrently as controls primary mouse hepatocytes were isolated from WT C57BL/6J mice and human hepatocyte cell lines were raised. Oligonucleotide compositions targeting UGP2 or EEF1A1 mRNA (see Table 1) were delivered under gymnotic free uptake conditions into the isolated hepatocytes. After 48 hours, total RNA was collected using the a suitable technology (e.g., SV 96 Total RNA Isolation System Protocol (Promega: Z3505)). RT-PCR using High-Capacity cDNA Reverse Transcription Kit with random hexamer primers (Life Technologies, Cat. No. 4388950) was used to generate a cDNA library. Primers flanking the edit site of the endogenously expressed target RNA were used (see Table 4) to amplify cDNA species of interest using the Phusion High-Fidelity DNA Polymerase protocol (Thermo: F-530XL). The PCR product was Sanger-sequenced and the percent of ADAR mediated editing was calculated using the program EditR (see FIG. 24). Editing levels in primary hepatocytes isolated from huADAR p110 animals were higher than levels observed in primary hepatocytes isolated from WT mice, and are more similar to those observed in human cells than the levels observed in WT mice are.

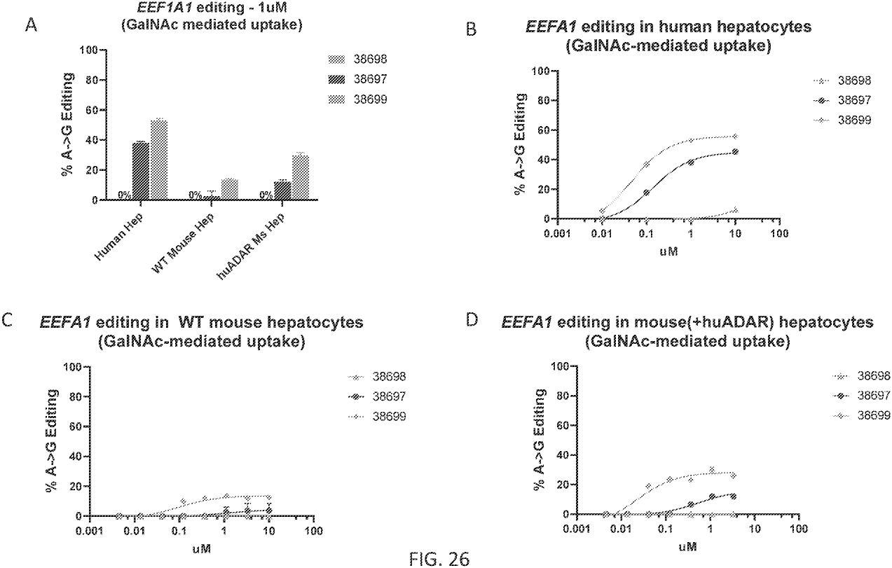

In some embodiments, oligonucleotides and compositions were assessed in primary mouse hepatocyte cells derived from huADAR1 p110 animals. Concurrently as controls primary mouse hepatocytes were isolated from WT C57BL/6J mice and human hepatocyte cell lines were raised. GalNac conjugated oligonucleotides targeting UGP2 or EEF1A1 mRNA (see table 1) were delivered for uptake into the isolated hepatocytes. After 48 hours, total RNA was collected using the SV 96 Total RNA Isolation System Protocol (Promega: Z3505). RT-PCR using High-Capacity cDNA Reverse Transcription Kit with random hexamer primers (Life Technologies, Cat. No. 4388950) was used to generate a cDNA library. Primers flanking the edit site of the endogenously expressed target RNA were used (see Table 4) to amplify cDNA species of interest using the Phusion High-Fidelity DNA Polymerase protocol (Thermo: F-530XL). The PCR product was Sanger-sequenced and the percent of ADAR mediated editing was calculated using the program EditR (see FIG. 25 and FIG. 26). Editing levels in primary hepatocytes isolated from huADAR p110 animals were higher than levels observed in primary hepatocytes isolated from WT mice, and are more similar to those observed in human cells than the levels observed in WT mice are (see FIG. 25 and FIG. 26).

In some embodiments, oligonucleotides and compositions are assessed in primary mouse hepatocyte cells derived from huADAR1 p150 animals. Concurrently as controls primary mouse hepatocytes are isolated from WT C57BL/6J mice and human hepatocyte cell lines are raised. Oligonucleotides targeting UGP2 or EEF1A1 mRNA (see Table 1) are delivered under Gymnotic free uptake conditions into the isolated hepatocytes. After 48 hours, total RNA is collected using the SV 96 Total RNA Isolation System Protocol (Promega: Z3505). RT-PCR using High-Capacity cDNA Reverse Transcription Kit with random hexamer primers (Life Technologies, Cat. No. 4388950) is used to generate a cDNA library. Primers flanking the edit site of the endogenously expressed target RNA are used (see Table 4) to amplify cDNA species of interest using the Phusion High-Fidelity DNA Polymerase protocol (Thermo: F-530XL). The PCR product is Sanger-sequenced and the percent of ADAR mediated editing is calculated using the program EditR. Editing levels in primary hepatocytes isolated from huADAR p 150 animals are higher than levels observed in primary hepatocytes isolated from WT mice, and are more similar to those observed in human cells than the levels observed in WT mice are.

In some embodiments, oligonucleotides and compositions are assessed in primary mouse hepatocyte cells derived from huADAR1 p150 animals. Concurrently as controls primary mouse hepatocytes are isolated from WT C57BL/6J mice and human hepatocyte cell lines are raised. GalNac conjugated oligonucleotides targeting UGP2 or EEF1A1 mRNA (see Table 1) were delivered for uptake into the isolated hepatocytes. After 48 hours, total RNA is collected using the SV 96 Total RNA Isolation System Protocol (Promega: Z3505). RT-PCR using High-Capacity cDNA Reverse Transcription Kit with random hexamer primers (Life Technologies, Cat. No. 4388950) is used to generate a cDNA library. Primers flanking the edit site of the endogenously expressed target RNA are used (see Table 4) to amplify cDNA species of interest using the Phusion High-Fidelity DNA Polymerase protocol (Thermo: F-530XL). The PCR product is Sanger-sequenced and the percent of ADAR mediated editing is calculated using the program EditR. Editing levels in primary hepatocytes isolated from huADAR p 150 animals are higher than levels observed in primary hepatocytes isolated from WT mice, and are more similar to those observed in human cells than the levels observed in WT mice are.

### Example 8 - In Vivo Editing of UGP2 in huADAR Transgenic Mice

Among other things, provided technologies, e.g., non-human animals engineered to comprise or express ADAR1 polypeptide or a characteristic portion thereof, are particularly useful for assessing agents for adenosine editing. In certain embodiments, editing agents were administered to engineered huADAR1 mice and specific tissues (e.g., CNS associated tissues) were harvested for editing analysis.

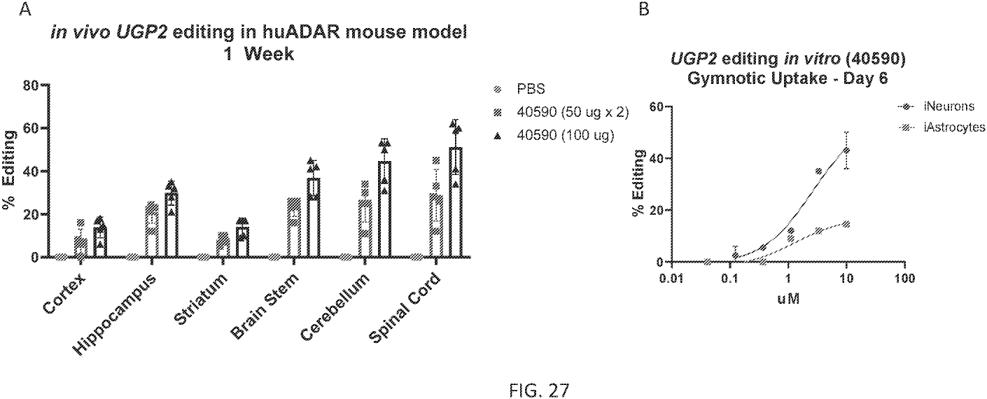

In some embodiments, oligonucleotide compositions targeting UDP-glucose pyrophosphorylase 2 (UGP2) or control vehicle were introduced to huADAR1 p110 mice through either a single intracerebroventricular (ICV) injection of 100 µg oligonucleotide or two ICV injections (day 0 and day 2) of 50 µg each (total 100 µg). On day 6 mice were sacrificed for immediate biopsy. CNS samples were harvested from huADAR p110 animals; samples were further divided by known spatial/functional delineations (e.g., Cortex, Hippocampus, Striatum, Brain Stem, Cerebellum, and Spinal Cord) and total RNA was then collected from each sample using a suitable technology (e.g., SV 96 Total RNA Isolation System Protocol (Promega: Z3505)). RT-PCR using High-Capacity cDNA Reverse Transcription Kit with random hexamer primers (Life Technologies, Cat. No. 4388950) was used to generate a cDNA library. Primers flanking the edit site of the endogenously expressed target RNA were used (see Table 4) to amplify cDNA species of interest using the Phusion High-Fidelity DNA Polymerase protocol (Thermo: F-530XL). The PCR product was Sanger-sequenced and the percent of ADAR mediated editing was calculated using the program EditR. Various levels of in vivo editing were observed. Editing activities were also observed in human iNeurons and/or iAstrocytes (gymnotic oligonucleotide uptake, measured at day 6) (see FIG. 27).

### Example 9 - In Vivo Editing of SRSF1 in huADAR Transgenic Mice

Among other things, provided technologies, e.g., non-human animals engineered to comprise or express ADAR1 polypeptide or a characteristic portion thereof, are particularly useful for assessing agents for adenosine editing. In certain embodiments, specific tissues (e.g., CNS associated tissues) were harvested from huADAR1 transgenic animals and editing by oligonucleotide compositions were assessed.

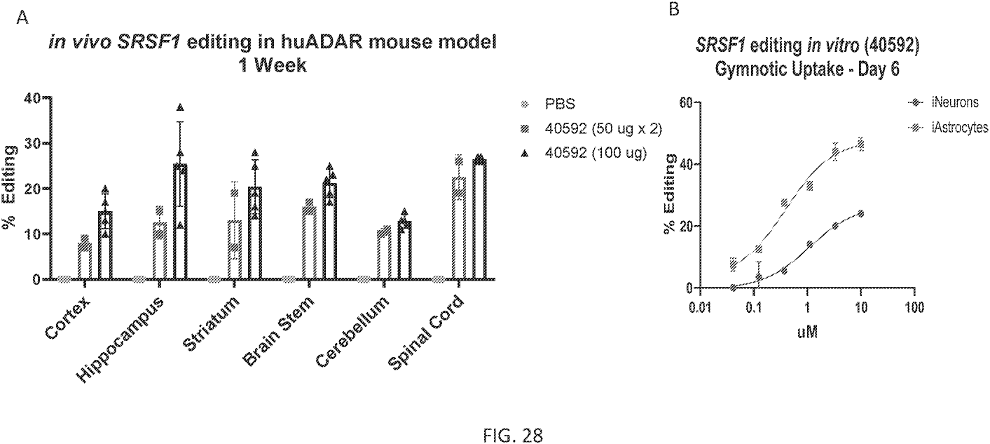

In some embodiments, oligonucleotide compositions targeting serine and arginine rich splicing factor 1 (SRSF1) or control vehicle were introduced to huADAR1 p110 mice through either a single intracerebroventricular (ICV) injection of 100 ug oligonucleotide or two ICV injections (day 0 and day 2) of 50 ug. On day 6 mice were sacrificed for immediate biopsy. CNS samples were harvested from huADAR p110 animals; samples were further divided by known spatial/functional delineations (e.g., Cortex, Hippocampus, Striatum, Brain Stem, Cerebellum, and Spinal Cord) and total RNA was then collected from each sample using a suitable technology (e.g., SV 96 Total RNA Isolation System Protocol (Promega: Z3505)). RT-PCR using High-Capacity cDNA Reverse Transcription Kit with random hexamer primers (Life Technologies, Cat. No. 4388950) was used to generate a cDNA library. Primers flanking the edit site of the endogenously expressed target RNA were used (for primer information see Table 4) to amplify cDNA species of interest using the Phusion High-Fidelity DNA Polymerase protocol (Thermo: F-530XL). The PCR product was Sanger-sequenced and the percent of ADAR mediated editing was calculated using the program EditR. Various levels of in vivo editing were observed. Editing activities were also observed in human iNeurons and/or iAstrocytes (gymnotic oligonucleotide uptake, measured at day 6) (See FIG. 28).

### Example 10 - In Vivo Editing of SERPINA1Pi*Z in huADAR Transgenic Mice

As described herein, animals engineered to comprise an ADAR1 polypeptide or a characteristic portion thereof, or to comprise and/or express a polynucleotide whose sequence encodes an ADAR1 polypeptide or a characteristic portion thereof, may be crossed with various animals (e.g., model animals of various conditions, disorders or diseases) to provide, among other things, animal models which comprise both characteristic elements associated with various conditions, disorders or diseases, and an ADAR1 polypeptide or a characteristic portion thereof or a polynucleotide whose sequence encodes an ADAR1 polypeptide or a characteristic portion thereof. In some embodiments, an animal is a model animal comprising SERPINA1-Pi*Z. In some embodiments, an animal comprises 1024 G>A (E342K) mutation of human SERPINA1 and a polynucleotide whose sequence encodes an ADAR1 polypeptide or a characteristic portion thereof. Among other things, such animals are useful for assessing various agents, e.g., oligonucleotides, for editing 1024 G>A (E342K) mutation of human SERPINA1. Among other things, provided technologies, e.g., non-human animals engineered to comprise or express ADAR1 polypeptide or a characteristic portion thereof, are particularly useful for assessing agents for adenosine editing.

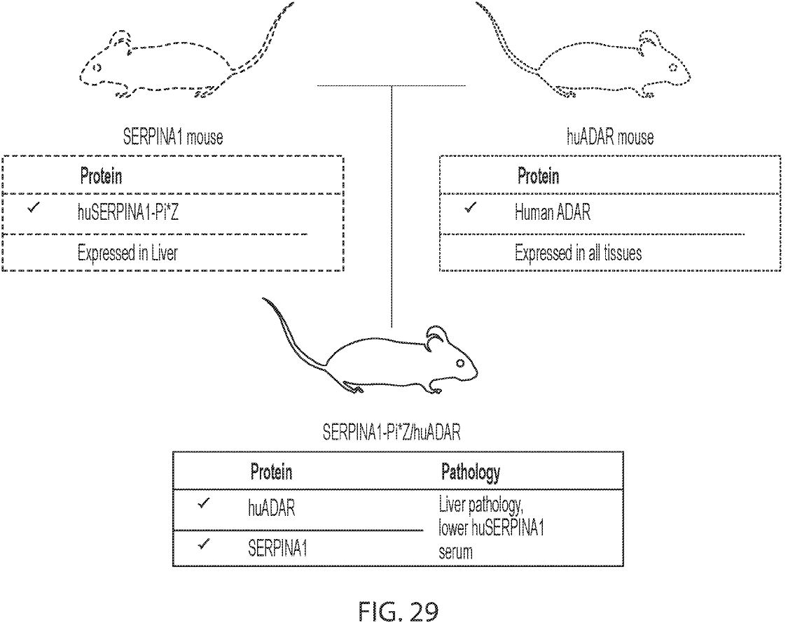

In some embodiments, a huADAR mouse as described herein is crossed with another mouse comprising a nucleotide sequence of interest (e.g., a mutation associated with a condition, disorder or disease). In certain embodiments, such a cross is performed using in vitro fertilization as is known in the art in accordance with the present disclosure. In certain embodiments, such a mouse comprises a human serpin family A member 1 (SERPINA1) polynucleotide sequence or a characteristic portion thereof. In certain embodiments, such a mouse is a SERPINA1-Pi*Z mouse, comprising a human SERPINA1 gene comprising a G to A mutation that corresponds to a 1024 G>A (E342K) mutation. In some embodiments, resultant offspring comprise both a human SERPINA1-Pi*Z polynucleotide sequence or a characteristic portion thereof (e.g., a portion comprising a mutation, e.g., 1024 G>A associated with a condition, disorder or disease) and a huADAR1 polynucleotide sequence or a fragment thereof (See FIG. 29). In some embodiments, double transgenic animals (e.g., comprising a human ADAR1 sequence or a characteristic portion thereof and a sequence associated with a condition, disorder or disease) may also comprise additional background mutations or alleles in heterozygous, hemizygous, and/or homozygous form that render them humanized (i.e. with immunodeficient phenotypes), such genotypes include but are not limited to NOD.Cg-Prkdc^{scid} Il2rgtm1^{Wjl}/SzJ, or NOD/ShiLtJ, alternative suitable humanized mouse strains are known in the art. In some embodiments, a mouse comprising a polynucleotide whose sequence encodes an ADAR1 polypeptide or a characteristic portion thereof is crossed with a mouse comprising a SERPINA1 mutation (e.g., 1024 G>A associated with a condition, disorder or disease (e.g., alpha 1-antitrypsin (A1AT) deficiency)). In some embodiments, a second mouse crossed with is The Jackson Laboratory Stock No: 028842; NSG-PiZ (see also Borel F; Tang Q; Gernoux G; Greer C; Wang Z; Barzel A; Kay MA; Shultz LD; Greiner DL; Flotte TR; Brehm MA; Mueller C. 2017. Survival Advantage of Both Human Hepatocyte Xenografts and Genome-Edited Hepatocytes for Treatment of alpha-1 Antitrypsin Deficiency. Mol Ther 25(11):2477-2489PubMed: 29032169MGI: J:243726, and Li S; Ling C; Zhong L; Li M; Su Q; He R; Tang Q; Greiner DL; Shultz LD; Brehm MA; Flotte TR; Mueller C; Srivastava A; Gao G. 2015. Efficient and Targeted Transduction of Nonhuman Primate Liver With Systemically Delivered Optimized AAV3B Vectors. Mol Ther 23(12):1867-76PubMed: 26403887MGI: J:230567). As described herein, in some embodiments, a huADAR mouse is engineered to comprise and/or express a polynucleotide whose sequence encodes a human ADAR1 p110 polypeptide or a characteristic portion thereof. In some embodiments, a huADAR mouse is engineered to comprise and/or express a polynucleotide whose sequence encodes a human ADAR1 p150 polypeptide or a characteristic portion thereof.

In some embodiments, a huADAR mouse as described herein was crossed with another mouse comprising a nucleotide sequence of interest. In some embodiments, a mouse comprising a polynucleotide whose sequence encoded an ADAR1 polypeptide was crossed with a mouse comprising a SERPINA1 mutation (e.g., 1024 G>A associated with a condition, disorder or disease (e.g., alpha 1-antitrypsin (A1AT) deficiency)). In some embodiments, such a cross was performed using in vitro fertilization as is known in the art in accordance with the present disclosure. In some embodiments, such a mouse comprised a human serpin family A member 1 (SERPINA1) polynucleotide sequence or a characteristic portion thereof. In some embodiments, such a mouse was a SERPINA1-Pi*Z mouse, comprising a human SERPINA1 gene comprising a G to A mutation that corresponds to, e.g., a 1024 G>A (E342K) mutation, or a genetic feature corresponding thereto. In some embodiments, resultant offspring comprised both a human SERPINA1-Pi*Z polynucleotide sequence and a huADAR1 polynucleotide sequence (e.g., see FIG. 29). In some embodiments, double transgenic animals also comprised additional background mutations or alleles in heterozygous, hemizygous, and/or homozygous (wild type or mutant) form, that in mutant form render them humanized (e.g., with immunodeficient phenotypes). In some embodiments, such genotypes included NOD.Cg-Prkdc^{scid} Il2rgtm1^{Wjl}/SzJ.

As appreciated by those skilled in the art, various technologies may be utilized for cross breeding in accordance with the present disclosure. In some embodiments, a technology is or comprises IVF (e.g., using sperms of a heterozygous or homozygous huADAR mouse and oocytes from another mouse, or vice versa). In some embodiments, a technology is or comprises natural breeding (e.g., using sperms of a heterozygous or homozygous huADAR mouse and oocytes from another mouse, or vice versa).

For example, in some embodiments, heterozygous sperms from a huADAR male mice and oocytes from NOD.Cg-Prkdcscid Il2rgtm1Wjl Tg(SERPINA1*E342K)#Slcw/SzJ (NSG-PiZ, Stock #028842) female mice are utilized via, e.g., IVF, to generate Prkdcscid heterozygous / Il2rgtm1Wj1 heterozygous / Tg(SERPINA1*E342K)#Slcw heterozygous / hADAR heterozygous female mice and Prkdcscid heterozygous / Il2rgtm1Wjl hemizygous / Tg(SERPINA1*E342K)#Slcw heterozygous / hADAR heterozygous male mice. In some embodiments, homozygous sperms from a huADAR male mice and oocytes from NOD.Cg-Prkdcscid Il2rgtm1Wjl Tg(SERPINA1*E342K)#Slcw/SzJ (NSG-PiZ, Stock #028842) female mice are utilized via, e.g., IVF, to generate Prkdcscid heterozygous / Il2rgtm1Wjl heterozygous / Tg(SERPINA1*E342K)#Slcw heterozygous / hADAR heterozygous female mice and Prkdcscid heterozygous / Il2rgtm1Wjl hemizygous / Tg(SERPINA1*E342K)#Slcw heterozygous / hADAR heterozygous male mice. In some embodiments, homozygous sperm from strain “hADAR” male mice and oocytes from NOD.Cg-Prkdcscid Il2rgtm1Wjl Tg(SERPINA1*E342K)#Slcw/SzJ (NSG-PiZ, Stock #028842) female mice are utilized, and resulting mice are crossed to, e.g., NOD/ShiLtJ (The Jackson Laboratory Stock #001976) mice to establish a series of colonies. In some embodiments, generated mice are (assuming the Prkdcscid / Il2rgtm1Wjl / Tg(SERPINA1*E342K)#Slcw / hADAR gene order) HET HET HET HET, HET WILD HET HET, WILD HET HET HET, WILD WILD HET HET, HET HEMI HET HET, HET HEMI HET WILD, HET HET HET WILD, and/or WILD HEMI HET HET. One skilled in the art appreciates that male or female gametes may be donated from either strain e.g., that in some embodiments oocytes may be donated from huADAR lines, while sperm may be donated from the other genotype, e.g., NOD.Cg-Prkdc^{scid} I12rg^{tm1WJl} Tg(SERPINA1*E342K)#Slcw/SzJ (NSG-PiZ, Stock #028842). In some embodiments, a huADAR (or hADAR) mice is engineered to comprise and/or express a polynucleotide whose sequence encodes an ADAR1 polypeptide or a characteristic portion thereof. In some embodiments, an animal comprises a polynucleotide whose sequence encodes an ADAR1 polypeptide or a characteristic portion thereof in its genome. In some embodiments, an animal comprises a polynucleotide whose sequence encodes an ADAR1 polypeptide or a characteristic portion thereof in its germline genome. In some embodiments, an ADAR1 polypeptide is human ADAR1. In some embodiments, a human ADAR1 is human ADAR1 p110. In some embodiments, a human ADAR1 is human ADAR1 p150. As examples, a number of animals comprising human ADAR1 p110 and 1024 G>A (E342K) mutation in human SERPINA1 were generated using one or more protocols described herein (e.g., using heterozygous hADAR1 sperms and IVF). As appreciated by those skilled in the art, in some embodiments, generated animals can be further bred to produce animals of desired genotypes, e.g., heterozygous, hemizygous, or homozygous mice. As appreciated by those skilled in the art, in some embodiments, animals comprising homozygous wild type alleles for one or more loci (e.g., Prkdcscid, I12rgtm1Wjl, Tg(SERPINA1*E342K)#Slcw, and/or hADAR) may provide appropriate relative controls.

In some embodiments, using IVF, heterozygous sperms from huADAR male mice and oocytes from NOD.Cg-Prkdcscid Il2rgtm1Wjl Tg(SERPINA1*E342K)#Slcw/Szj (NSG-PiZ, Stock #028842) female mice were crossed to generate Prkdcscid heterozygous / Il2rgtm1Wjl heterozygous / Tg(SERPINA1*E342K)#Slcw heterozygous / hADAR heterozygous female mice and Prkdcscid heterozygous / 12rgtm1Wjl hemizygous / Tg(SERPINA1*E342K)#Slcw heterozygous / hADAR heterozygous male mice. Additionally, pups were produced with genotypes (assuming the Prkdcscid / Il2rgtm1Wjl / Tg(SERPINA1*E342K)#Slcw / hADAR gene order) HET HET HET HET, HET WILD HET HET, WILD HET HET HET, WILD WILD HET HET, HET HEMI HET HET, HET HEMI HET WILD, HET HET HET WILD, and/or WILD HEMI HET HET. A number of animals comprising human ADAR1 p110 and 1024 G>A (E342K) mutation in human SERPINA1 were generated using one or more protocols described herein (e.g., using heterozygous hADAR1 sperms and IVF). In some embodiments, animals comprising homozygous wild type alleles for one or more loci (e.g., Prkdcscid, Il2rgtm1Wjl, Tg(SERPINAl*E342K)#Slcw, and/or hADAR) can act as appropriate relative controls.

In some embodiments, agents and compositions thereof, e.g., oligonucleotide compositions targeting a mutation in SERPINA1, e.g., SERPINA1-Pi*Z, are assessed using provided technologies. For example, in some embodiments, oligonucleotide compositions or control composition are introduced to double transgenic SERPINA1-Pi*Z / huADAR1 p110 mice, and optionally transgenic SERPINA1-Pi*Z mice and/or control WT mice through a dose schedule (e.g., a three-dose schedule on day 0, day 2, and day 4) at a suitable amount, e.g., 10 mg/kg of oligonucleotide in PBS delivered subcutaneously, after a period of time, e.g., on day 6, mice are sacrificed for immediate biopsy. Liver samples are harvested from double transgenic SERPINA1-Pi*Z / huADAR1 animals, transgenic SERPINA1-Pi*Z animals, and control WT animals; total RNA is then collected from the livers. As those skilled in the art appreciate, various technologies may be utilized in accordance with the present disclosure, e.g., the SV 96 Total RNA Isolation System Protocol (Promega: Z3505). RT-PCR, e.g., using High-Capacity cDNA Reverse Transcription Kit with random hexamer primers (Life Technologies, Cat. No. 4388950) is used to generate a cDNA library. Primers flanking the edit site of the SERPINA1-Pi*Z target RNA are used to amplify cDNA species of interest using, e.g., the Phusion High-Fidelity DNA Polymerase protocol (Thermo: F-530XL). The PCR product is Sanger-sequenced and the percent of ADAR mediated editing is calculated using, e.g., the program EditR. Those skilled in the art appreciate that various other sequencing technologies may also be utilized in accordance with the present disclosure. Allele specific PCR, qPCR, or ARMS assay may also be utilized for assessing editing. In some embodiments, different in vivo editing levels of SERPINA1-Pi*Z mRNA in double transgenic SERPINA1-Pi*Z / huADAR animals are observed when compared to transgenic SERPINA1-Pi*Z animals or WT animals. In some embodiments, assessment can be performed with double transgenic animals and without using transgenic SERPINA1-Pi*Z animals without utilizing hADAR1 or WT animals. In some embodiments, an oligonucleotide composition provides higher editing levels compared to a control composition. In some embodiments, a control composition is a control vehicle. In some embodiments, a control composition contain different oligonucleotides compared to an oligonucleotide composition to be assessed. In some embodiments, a control composition is a stereorandom composition. In some embodiments, an oligonucleotide composition to be assessed is a chirally controlled oligonucleotide composition.

Various oligonucleotide agents and compositions thereof, e.g., oligonucleotide compositions targeting a mutation in SERPINA1, e.g., SERPINA1-Pi*Z, were assessed using provided technologies. In some embodiments, test primary hepatocytes were derived from Prkdcscid heterozygous / Il2rgtm1Wjl heterozygous / Tg(SERPINA1*E342K)#Slcw heterozygous / hADAR heterozygous female mice and Prkdcscid heterozygous / 12rgtm1Wjl hemizygous / Tg(SERPINAl*E342K)#Slcw heterozygous / hADAR heterozygous male mice. In some embodiments, control primary hepatocytes were derived from Prkdcscid heterozygous / Il2rgtm1Wjl heterozygous / Tg(SERPINA1*E342K)#Slcw heterozygous / hADAR wild type female mice and Prkdcscid heterozygous / Il2rgtm1Wjl hemizygous / Tg(SERPINA1*E342K)#Slcw heterozygous / hADAR wild type male mice. In some embodiments, oligonucleotide compositions at various concentrations, e.g., 0.3 uM or 3 uM or control compositions were introduced to primary hepatocytes derived from double transgenic SERPINA1-Pi*Z / huADAR1 p110 mice for gymnotic uptake. RNA editing events can be analyzed using various technologies in accordance with the present disclosure. In some embodiments, SV 96 Total RNA Isolation System Protocol (Promega: Z3505) were utilized. In some embodiments, RT-PCR, e.g., using High-Capacity cDNA Reverse Transcription Kit with random hexamer primers (Life Technologies, Cat. No. 4388950) were used to generate a cDNA library. In some embodiments, primers flanking the edit site of the SERPINA1-Pi*Z target RNA were used to amplify cDNA species of interest using the Phusion High-Fidelity DNA Polymerase protocol (Thermo: F-530XL). In some embodiments, PCR product was Sanger-sequenced and the percent of ADAR mediated editing was calculated. Various oligonucleotide compositions provided higher editing levels when compared to a control composition and/or a composition with a lower concentration of oligonucleotide tested.

In some embodiments, A1AT protein concentrations in samples (e.g., from serum, blood, liver, etc. of animals treated with an agent or composition thereof (e.g., an oligonucleotide composition) and/or a control composition; for an example, see above) are analyzed. As those skilled in the art appreciate, various technologies may be utilized in accordance with the present disclosure, e.g., protein concentrations may be quantified by an Elisa Assay. A1AT protein concentration can be calculated using a A1AT ELISA e.g., Abcam - ab108799 following manufacturers instruction. In brief, standards are generated, e.g., using recombinant A1AT protein diluted to a suitable concentration, e.g., 25 ng/mL in a suitable diluent and serially diluted at a suitable fold, e.g., 2-fold for a number of points, e.g., 7 points. Protein samples are harvested from liver samples. Prepared standards and diluted protein sample are added to wells of a SERPINA1 antibody coated and blocked 96 well plate and incubated for 2 hours at room temperature. Plates are washed with provided ELISA wash buffer, e.g., 6 times (300ul/well) before a biotinylated SERPINA1 antibody is diluted to 1X in a suitable diluent and added to each well for a suitable length of time, e.g., 1 hour at room temperature. Wells are washed, and a streptavidin-peroxidase complex, diluted to 1X in a suitable diluent, is added to each well for a suitable period of time, e.g., 30 minutes at room temperature. Wells are washed a final time before 3,3′,5,5′-Tetramethylbenzidine (TMB) is added to each well and the plate is developed for a suitable period of time, e.g., 20 minutes before stop solution is added. The plate is then read at 450 nm and 570 nm. A reading at 570 nm is subtracted from a 450 nm reading to account for optical imperfections and the plate is quantified. In some embodiments, levels of SERPINA1-Pi*Z protein is higher in double transgenic SERPINA1-Pi*Z / huADAR animals when compared to transgenic SERPINA1-Pi*Z animals or WT animals. In some embodiments, assessment can be performed with double transgenic animals and without using transgenic SERPINA1-Pi*Z animals without utilizing hADAR1 or WT animals. In some embodiments, an oligonucleotide composition provides higher editing levels and/or higher levels of A1AT protein and/or activities compared to a control composition. In some embodiments, an oligonucleotide composition provides higher levels/activities of A1AT compared to a control composition. In some embodiments, an oligonucleotide composition provides higher levels of properly folded and/or secreted A1AT protein and/or activities compared to a control composition. In some embodiments, an oligonucleotide composition provides higher levels of A1AT protein and/or activities compared to a control composition. As appreciated by those skilled in the art, levels, properties and/or activities, including sequences, may also be assessed using other technologies such as mass spectrometry. In some embodiments, LC-MS based proteomics technologies are utilized to quantitate A1AT proteins (e.g., wild-type and/or mutant proteins (e.g., encoded by RNA with or without editing)). In some embodiments, A1AT protein in a biological sample (e.g., from animals treated with an agent or composition thereof (e.g., an oligonucleotide composition) and/or a control composition; for an example, see above) are analyzed. For example, in some embodiments, A1AT protein in a biological sample such as at least one tissue (e.g., liver, kidney, muscle, and/or heart etc.), or biological liquids (e.g., serum, and/or blood etc.) from animals treated with an RNA editing modifying agent (e.g., from animals as described herein treated with an agent or composition described herein (e.g., an oligonucleotide composition) and/or a control composition; for an example, see above) are analyzed. In some embodiments, levels, properties and/or activities of A1AT protein in serum or blood are assessed.

In some embodiments, a control composition is a control vehicle. In some embodiments, a control composition contain different oligonucleotides compared to an oligonucleotide composition to be assessed. In some embodiments, a control composition is a stereorandom composition. In some embodiments, an oligonucleotide composition to be assessed is a chirally controlled oligonucleotide composition.

### EQUIVALENTS

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, configurations, etc. described herein are meant to be example and that actual parameters, dimensions, materials, and/or configurations, etc. will depend upon the specific application or applications for which the teachings of the present disclosure is/are used. Those skilled in the art will recognize, or be able to ascertain using no more than routine experimentation, many equivalents to the specific embodiments described in the present disclosure. It is, therefore, to be understood that the foregoing embodiments are presented by way of example only and that, within the scope of appended claims and equivalents thereto, claimed technologies may be practiced otherwise than as specifically described and claimed. In addition, any combination of two or more features, systems, articles, materials, kits, and/or methods, etc., if such features, systems, articles, materials, kits, and/or methods, etc., are not mutually inconsistent, is included within the scope of the present disclosure.

1. A non-human animal engineered to comprise a human ADAR1 polypeptide or a characteristic portion thereof, a non-human animal engineered to comprise and/or express a polynucleotide whose sequence encodes a human ADAR1 polypeptide or a characteristic portion thereof.

2. A non-human animal engineered to comprise an ADAR1 polypeptide or a characteristic portion thereof, or a non-human animal engineered to comprise and/or express a polynucleotide whose sequence encodes an ADAR1 polypeptide or a characteristic portion thereof.

3. The animal of any one of the preceding claims, wherein the genome of the animal comprises a polynucleotide whose sequence encodes an ADAR1 polypeptide or a characteristic portion thereof.

4. The animal of any one of the preceding claims, wherein the germline genome of the animal comprises a polynucleotide whose sequence encodes an ADAR1 polypeptide or a characteristic portion thereof.

5. The animal of any one of the preceding claims, wherein the animal is engineered to express an ADAR1 polypeptide or a characteristic portion thereof.

6. The animal of any one of the preceding claims, wherein expression of the ADAR1 polypeptide or a characteristic portion thereof is inducible in one or more cells and/or tissues.

7. The animal of any one of the preceding claims, wherein expression of the ADAR1 polypeptide or a characteristic portion thereof is constitutive in one or more cells and/or tissues.

8. The animal of any one of the preceding claims, wherein expression of the ADAR1 polypeptide or a characteristic portion thereof is tissue specific.

9. The animal of any one of the preceding claims, wherein the animal is a rodent.

10. The animal of any one of the preceding claims, wherein the animal is a mouse.

11. The animal of any one of the preceding claims, wherein the animal is a rat.

12. The animal of any one of the preceding claims, wherein the ADAR1 polypeptide or a characteristic portion thereof is or comprises a deaminase domain.

13. The animal of any one of the preceding claims, wherein the ADAR1 polypeptide or a characteristic portion thereof is or comprises one or more (e.g., 1, 2, 3 or more) dsRBDs.

14. The animal of any one of the preceding claims, wherein the ADAR1 polypeptide or a characteristic portion thereof is or comprises one or more (e.g., 1, 2, 3 or more) Z-DNA binding domains.

15. The animal of any one of the preceding claims, wherein the amino acid sequence of the ADAR1 polypeptide or a characteristic portion thereof is or comprises an amino acid sequence of a primate ADAR1 polypeptide or a fragment thereof, an amino acid sequence of a primate ADAR1 polypeptide or a characteristic portion thereof, and/or an amino acid sequence of a primate ADAR1 polypeptide, or the amino acid sequence of the ADAR1 polypeptide or a characteristic portion thereof is an amino acid sequence of a primate ADAR1 polypeptide.

16. The animal of any one of the claims 2-15, wherein the ADAR1 polypeptide or a characteristic portion thereof is a primate ADAR1 polypeptide.

17. The animal of any one of the claims 2-16, wherein the primate ADAR1 polypeptide is a human ADAR1 polypeptide.

18. The animal of any one of the preceding claims, wherein the primate ADAR1 polypeptide is the p 110 isoform of human ADAR1.

19. The animal of any one of claims 1-17, wherein the primate ADAR1 polypeptide is the p150 isoform of human ADAR1.

20. The animal of any one of the preceding claims, wherein the ADAR1 polypeptide or a characteristic portion thereof, when contacted with an oligonucleotide composition targeting a target adenosine, provides increased editing level of the target adenosine (“engineered editing level”) in one or more cells or tissues of the engineered animal compared to that observed in the corresponding cells or tissues in a reference animal (“reference editing level”), wherein the reference animal is not engineered to express the ADAR1 polypeptide or a characteristic portion thereof.

21. The animal of claim 20, wherein the oligonucleotide composition is of WV-38700, WV-38702, or WV-40590, and the target adenosine is its targeted adenosine in a human or mouse UGP2 transcript, and/or wherein the oligonucleotide composition is of WV-40592, and the target adenosine is its targeted adenosine in a human or mouse SRSF1 transcript, and/or wherein the oligonucleotide composition is of WV-38697, or WV-38699, and the target adenosine is its targeted adenosine in a human or mouse EEF1A1 transcript.

22. The animal of any one of the preceding claims, wherein the one or more cells or tissues are liver tissue, or wherein the one or more cells or tissues are mouse hepatocytes.

23. The animal of any one of the preceding claims, wherein the engineered editing level is about or at least about 1.5, 2, 3, 4, 5, 6, 7, 8, 9, 10, 15, 20, 30, 40, 50, 100 or more fold of the reference editing level.

24. The animal of any one of the preceding claims, wherein the animal is heterozygous.

25. The animal of any one of claims 1-24, wherein the animal is homozygous.

26. A non-human embryo engineered to comprise an ADAR1 polypeptide or a characteristic portion thereof, or a non-human embryo engineered to comprise and/or express a polynucleotide whose sequence encodes an ADAR1 polypeptide or a characteristic portion thereof.

27. The embryo of any one of the preceding claims, wherein the genome of the embryo comprises a polynucleotide whose sequence encodes an ADAR1 polypeptide or a characteristic portion thereof, and/or wherein the embryo is engineered to express an ADAR1 polypeptide or a characteristic portion thereof, and/or wherein the ADAR1 polypeptide or a characteristic portion thereof is or comprises a deaminase domain, and/or wherein the ADAR1 polypeptide or a characteristic portion thereof is or comprises one or more (e.g., 1, 2, 3 or more) dsRBDs, and/or one or more (e.g., 1, 2, 3 or more) Z-DNA binding domains.

28. The embryo of any one of the preceding claims, wherein the amino acid sequence of the ADAR1 polypeptide or a characteristic portion thereof is or comprises an amino acid sequence of a primate ADAR1 polypeptide or a fragment thereof, optionally wherein the primate ADAR1 polypeptide is a human ADAR1 polypeptide, or wherein the primate ADAR1 polypeptide is the p 110 isoform of human ADAR1, or is the p150 isoform of human ADAR1.

29. The embryo of any one of the preceding claims, wherein the ADAR1 polypeptide or a characteristic portion thereof, when contacted with an oligonucleotide composition targeting a target adenosine, provides increased editing level of the target adenosine (“engineered editing level”) in one or more cells and/or tissues engineered to express the ADAR1 polypeptide or a characteristic portion thereof compared to that observed in reference cells and/or tissues not engineered to express the ADAR1 polypeptide or a characteristic portion thereof, optionally wherein the oligonucleotide composition is of WV-38700, WV-38702, or WV-40590, and the target adenosine is its targeted adenosine in a human or mouse UGP2 transcript, and/or wherein the oligonucleotide composition is of WV-40592, and the target adenosine is its targeted adenosine in a human or mouse SRSF1 transcript, and/or wherein the oligonucleotide composition is of WV-38697, or WV-38699, and the target adenosine is its targeted adenosine in a human or mouse EEF1A1 transcript, and optionally wherein the engineered editing level is about or at least about 1.5, 2, 3, 4, 5, 6, 7, 8, 9, 10, 15, 20, 30, 40, 50, 100 or more fold of the reference editing level.

30. The embryo of any one of the preceding claims, wherein the embryo is heterozygous.

31. The embryo of any one of claims 26-30, wherein the embryo is homozygous.

32. A cell engineered to comprise an ADAR1 polypeptide or a characteristic portion thereof, or a cell engineered to comprise and/or express a polynucleotide whose sequence encodes an ADAR1 polypeptide or a characteristic portion thereof.

33. The cell of any one of the preceding claims, wherein the cell is a non-human cell.

34. The cell of any one of the preceding claims, wherein the cell is an embryonic stem cell.

35. The cell of any one of the preceding claims, wherein the genome of the cell comprises a polynucleotide whose sequence encodes an ADAR1 polypeptide or a characteristic portion thereof.

36. The cell of any one of the preceding claims, wherein the ADAR1 polypeptide or a characteristic portion thereof is or comprises a deaminase domain.

37. The cell of any one of the preceding claims, wherein the ADAR1 polypeptide or a characteristic portion thereof is or comprises one or more (e.g., 1, 2, 3 or more) dsRBDs, and/or one or more (e.g., 1, 2, 3 or more) Z-DNA binding domains.

38. The cell of any one of the preceding claims, wherein the amino acid sequence of the ADAR1 polypeptide or a characteristic portion thereof is or comprises an amino acid sequence of a primate ADAR1 polypeptide or a fragment thereof.

39. The cell of any one of the preceding claims, wherein the amino acid sequence of the ADAR1 polypeptide or a characteristic portion thereof is or comprises an amino acid sequence of a primate ADAR1 polypeptide or a characteristic portion thereof.

40. The cell of any one of the preceding claims, wherein the primate ADAR1 polypeptide is a human ADAR1 polypeptide.

41. The cell of any one of the preceding claims, wherein the primate ADAR1 polypeptide is the p 110 isoform of human ADAR1, or is the p150 isoform of human ADAR1.

42. The cell of any one of the preceding claims, wherein the ADAR1 polypeptide or a characteristic portion thereof, when contacted with an oligonucleotide composition targeting a target adenosine, provides increased editing level of the target adenosine (“engineered editing level”) in one or more cells and/or tissues engineered to express the ADAR1 polypeptide or a characteristic portion thereof compared to that observed in reference cells and/or tissues not engineered to express the ADAR1 polypeptide or a characteristic portion thereof.

43. The cell of claim 42, wherein the oligonucleotide composition is of WV-38700, WV-38702, or WV-40590, and the target adenosine is its targeted adenosine in a human or mouse UGP2 transcript, and/or wherein the oligonucleotide composition is of WV-40592, and the target adenosine is its targeted adenosine in a human or mouse SRSF1 transcript, and/or wherein the oligonucleotide composition is of WV-38697, or WV-38699, and the target adenosine is its targeted adenosine in a human or mouse EEF1A1 transcript.

44. The cell of any one of the preceding claims, wherein the one or more cells or tissues are liver tissue.

45. The cell of any one of the preceding claims, wherein the one or more cells or tissues are mouse hepatocytes.

46. The cell of any one of the preceding claims, wherein the engineered editing level is about or at least about 1.5, 2, 3, 4, 5, 6, 7, 8, 9, 10, 15, 20, 30, 40, 50, 100 or more fold of the reference editing level.

47. The cell of any one of the preceding claims, wherein the cell is heterozygous.

48. The cell of any one of claims 32-46, wherein the cell is homozygous.

49. The animal, embryo or cell of any one of the preceding claims, wherein the amino acid sequence of an ADAR1 polypeptide or a characteristic portion thereof is or comprises a sequence that is the same as, differs by no more than 1-10 (e.g., 1-8, 1-5, 1, 2, 3, 4, 5, 6, 7, 8, 9, or 10) amino acids from, or has about or at least about 90%-100% (e.g., 90%, 91%, 92%, 93%, 94%, 95%, 96%, 97%, 98%, or 99%) homology with, SEQ ID NO: 27, 28, 29, 30, 31, 32, 33, 34, 35, 36, 37, 38, 39 or 40, and/or wherein the amino acid sequence of an ADAR1 polypeptide or a characteristic portion thereof is or comprises SEQ ID NO: 27, 28, 29, 30, 31, 32, 33, 34, 35, 36, 37, 38, 39 or 40, and/or wherein the amino acid sequence of an ADAR1 polypeptide or a characteristic portion thereof is or comprises SEQ ID NO: 5, 6, 9, 12, 16, 20, 26, 43, 46, 49, 52, or 55, or a characteristic portion thereof.

50. The animal, embryo or cell of any one of the preceding claims, wherein the animal, embryo or cell comprises a G to A mutation associated with a condition, disorder or disease.

51. The animal, embryo or cell of any one of the preceding claims, wherein the animal, embryo or cell comprises a G to A mutation that corresponds to 1024 G>A (E342K) mutation in human SERPINA1 gene.

52. A polynucleotide, comprising:
a) an ADAR1 polynucleotide encoding an ADAR1 polypeptide or a characteristic portion thereof,
b) a 5′ homology arm upstream of an ADAR1 polynucleotide, and
c) a 3′ homology arm downstream an ADAR1 polynucleotide,
wherein the 5′ and 3′ homology arms independently comprise a nucleotide sequence corresponding to a 5′ and 3′ target sequence in a non-human animal genome, respectively, wherein the 5′-target sequence is upstream of the 3′ target sequence.

53. The polynucleotide of any one of the preceding claims, wherein the ADAR1 polynucleotide is codon optimized to express an ADAR1 polypeptide or a characteristic portion thereof in an animal host cell.

54. The polynucleotide of claim 53, wherein the animal is a mouse or rat.

55. The polynucleotide of any one of the preceding claims, wherein the ADAR1 polypeptide or a characteristic portion thereof is or comprises a deaminase domain.

56. The polynucleotide of any one of the preceding claims, wherein the ADAR1 polypeptide or a characteristic portion thereof is or comprises one or more (e.g., 1, 2, 3 or more) dsRBDs and/or one or more (e.g., 1, 2, 3 or more) Z-DNA binding domains.

57. The polynucleotide of any one of the preceding claims, wherein the amino acid sequence of the ADAR1 polypeptide or a characteristic portion thereof is or comprises an amino acid sequence of a primate ADAR1 polypeptide or a fragment thereof.

58. The polynucleotide of any one of the preceding claims, wherein the primate ADAR1 polypeptide is a human ADAR1 polypeptide.

59. The polynucleotide of any one of the preceding claims, wherein the primate ADAR1 polypeptide is the p110 isoform of human ADAR1, or is the p150 isoform of human ADAR1.

60. The polynucleotide of any one of the preceding claims, wherein the ADAR1 polypeptide or a characteristic portion thereof, when contacted with an oligonucleotide composition targeting a target adenosine, provides increased editing level of the target adenosine (“engineered editing level”) in one or more cells and/or tissues engineered to express the ADAR1 polypeptide or a characteristic portion thereof compared to that observed in reference cells and/or tissues not engineered to express the ADAR1 polypeptide or a characteristic portion thereof.

61. The polynucleotide of claim 60, wherein the oligonucleotide composition is of WV-38700, WV-38702, or WV-40590, and the target adenosine is its targeted adenosine in a human or mouse UGP2 transcript, and/or wherein the oligonucleotide composition is of WV-40592, and the target adenosine is its targeted adenosine in a human or mouse SRSF1 transcript, and/or wherein the oligonucleotide composition is of WV-38697, or WV-38699, and the target adenosine is its targeted adenosine in a human or mouse EEF1A1 transcript.

62. The polynucleotide of any one of the preceding claims, wherein the polynucleotide has or comprises a sequence that encodes an ADAR1 polypeptide or a characteristic portion thereof of any one of the preceding claims, and/or wherein the sequence of the polynucleotide is or comprises SEQ ID NO: 2, 3, 4, 7, 8, 10, 11, 13, 14, 15, 17, 18, 19, 21, 22, 23, 24, 25, 41, 42, 44, 45, 47, 48, 50, 51, 53, or 54, or a characteristic portion thereof.

63. The polynucleotide of any one of the preceding claims, wherein the engineered editing level is about or at least about 1.5, 2, 3, 4, 5, 6, 7, 8, 9, 10, 15, 20, 30, 40, 50, 100 or more fold of the reference editing level.

64. The animal, embryo or cell of any one of the preceding claims, comprising and/or expressing a polynucleotide of any one of the preceding claims.

65. A vector comprising or expressing an ADAR1 polynucleotide encoding an ADAR1 polypeptide or a characteristic portion thereof, or a vector, comprising and/or expressing:
a) an ADAR1 polynucleotide encoding an ADAR1 polypeptide or a characteristic portion thereof,
b) a 5′ homology arm upstream of an ADAR1 polynucleotide, and
c) a 3′ homology arm downstream an ADAR1 polynucleotide,
wherein the 5′ and 3′ homology arms independently comprise a nucleotide sequence corresponding to a 5′ and 3′ target sequence in a non-human animal genome, respectively, wherein the 5′-target sequence is upstream of the 3′ target sequence; or
a vector, or the vector of claim 70, wherein the vector comprises and/or expresses a polynucleotide of any one of the preceding claims.

66. A population of cells, comprising a plurality of cells of any one of the preceding claims, wherein each cell is independently a cell of any one of the preceding claims.

67. A method, comprising introducing a polynucleotide or vector of any one of the preceding claims into a cell, embryo or animal.

68. The method of claim 67, wherein the cell, embryo or animal, prior to the introducing, does not comprise or express an ADAR1 polynucleotide, or does not express a primate or human ADAR1 polynucleotide, and/or wherein the cell embryo or animal is rodent, preferably mouse or rat.

69. A method of making a non-human animal, comprising:
(a) introducing an ADAR1 polynucleotide encoding an ADAR1 polypeptide or a characteristic portion thereof into the genome of an ES cell of a non-human animal; and
(b) generating a non-human animal using the ES cell generated in step (a); or
a method of making a non-human animal, comprising:
(a) introducing a polynucleotide of any one of the preceding claims into the genome of an ES cell of a non-human animal; and
(b) generating a non-human animal using the ES cell generated in step (a); or
a method of making a non-human animal, comprising:
(a) introducing an ADAR1 polynucleotide encoding an ADAR1 polypeptide or a characteristic portion thereof into the genome of a zygote of a non-human animal; and
(b) generating a non-human animal using the zygote generated in step (a); or
a method of making a non-human animal, comprising:
(a) introducing a polynucleotide of any one of the preceding claims into the genome of a zygote of a non-human animal; and
(b) generating a non-human animal using the zygote generated in step (a); or
a method of making a genetically modified ES cell of a non-human animal comprising:
introducing an ADAR1 polynucleotide encoding an ADAR1 polypeptide or a characteristic portion thereof into the genome of an ES cell of a non-human animal; or
a method of making a genetically modified ES cell of a non-human animal comprising:
introducing a polynucleotide of any one of the preceding claims into the genome of an ES cell of a non-human animal; or
a method for generating an engineered cell, comprising:
introducing an ADAR1 polynucleotide encoding an ADAR1 polypeptide or a characteristic portion thereof into a cell of a non-human animal; or
a method for generating an engineered cell, comprising:
introducing a polynucleotide of any one of the preceding claims into a cell of a non-human animal.

70. The method of any one of the preceding claims, comprising steps of:
contacting the genome of a cell with a nuclease to create a DNA break; and
introducing a polynucleotide of any one of the preceding claims.

71. The method of claim 70, wherein the nuclease is CRISPR/Cas9, a zinc finger nuclease, or a transcriptional activator-like effector nuclease.

72. A method for generating an animal, embryo or cell, comprising introducing to a first animal, embryo or cell a polynucleotide whose sequence encodes an ADAR1 polypeptide or a characteristic portion thereof.

73. The method of claim 72, wherein the polynucleotide is a polynucleotide of any one of the preceding claims.

74. A method for generating an animal, embryo or cell, comprising breeding a first animal with a second animal, wherein the second animal is an animal of any one of the preceding claims.

75. The method of any one of claims 72-74, wherein a first animal, embryo or cell is or comprises a cell, tissue or organ associated with or of a condition, disorder or disease.

76. A method for generating an animal, embryo or cell, comprising introducing a first cell, tissue or organ associated with or of a condition, disorder or disease to a second animal, embryo or cell of any one of the preceding claims.

77. A method for assessing an agent or a composition thereof for use in adenosine editing, comprising steps of:
administering an agent to an animal, embryo or cell of any one of the preceding claims.

78. The method of claim 77, wherein the agent or a composition thereof is an oligonucleotide composition; and/or wherein the agent or composition thereof provides a higher editing level in the engineered animal, embryo or cell (“engineered editing level”) compared to that in a reference animal, embryo or cell not engineered to express the ADAR1 polypeptide or a characteristic portion thereof (“reference editing level”).

79. A method, comprising expressing an RNA in an animal, embryo or cell of any one of the preceding claims, wherein a target adenosine of the RNA is edited.

80. The method of claim 79, comprising administering to the animal, embryo or cell an oligonucleotide or a composition thereof targeting the target adenosine.

81. The method of any one of claims 79-80, wherein the targeted adenosine is edited at a higher level (“engineered editing level”) compared to that in a reference animal, embryo or cell not engineered to express the ADAR1 polypeptide or a characteristic portion thereof (“reference editing level”) (e.g., wherein the engineered editing level is about or at least about 1.5, 2, 3, 4, 5, 6, 7, 8, 9, 10, 15, 20, 30, 40, 50, 100 or more fold of the reference editing level).

82. A method for characterizing an ADAR1 polypeptide or a characteristic portion thereof, comprising:
expressing an ADAR1 polypeptide or a characteristic portion thereof in an engineered host cell of an animal or an animal;
assessing expression level of an ADAR1 polypeptide or a characteristic portion thereof, or editing level of a target adenosine in a transcript.

83. The method of claim 82, wherein the ADAR1 polypeptide or a characteristic portion thereof is a human ADAR1 polypeptide or a characteristic portion thereof.

84. The method of any one of claims 82-83, wherein the animal is a non-human animal, or wherein the animal is a rodent, or wherein the animal is a mouse or rat.

85. The method of any one of claims 82-84, comprising assessing expression level of the ADAR1 polypeptide or a characteristic portion thereof, and/or assessing editing level of a target adenosine in a transcript.

86. The method of claim 85, comprising administering to the host cell or animal an oligonucleotide composition which targets the target adenosine.

87. The method of claim 86, wherein the oligonucleotide composition is of WV-38700, WV-38702, or WV-40590, and the target adenosine is its targeted adenosine in a human or mouse UGP2 transcript, and/or wherein the oligonucleotide composition is of WV-40592, and the target adenosine is its targeted adenosine in a human or mouse SRSF1 transcript, and/or wherein the oligonucleotide composition is of WV-38697, or WV-38699, and the target adenosine is its targeted adenosine in a human or mouse EEF1A1 transcript.

88. A method for characterizing an agent or an oligonucleotide or a composition, comprising:
administering the agent or oligonucleotide or composition to a cell or a population thereof comprising or expressing an ADAR1 polypeptide or a characteristic portion thereof, or a polynucleotide encoding an ADAR1 polypeptide or a characteristic portion thereof.

89. The method of claim 88, wherein the cell is a cell of any one of the preceding claims.

90. A method for characterizing an oligonucleotide or a composition, comprising:
administering the oligonucleotide or composition to a non-human animal or a population thereof comprising or expressing an ADAR1 polypeptide or a characteristic portion thereof, or a polynucleotide encoding an ADAR1 polypeptide or a characteristic portion thereof.

91. The method of claim 88, wherein the animal is an animal of any one of the preceding claims.

92. The method of any one of claims 88-91, wherein activity levels of an oligonucleotide or composition observed from a cell or a cell from an animal, or a population thereof, is more similar to those observed in a comparable human cell or a population thereof compared to those observed in a cell prior to engineering or a cell from an animal prior to engineering, or a population thereof, and/or wherein a comparable human cell is of the same type as a cell or a cell from an animal.

93. A method, comprising:
assessing an agent or a composition thereof in a cell, tissue or animal, wherein the cell, tissue or animal is or comprises a cell, tissue or organ associated or of a condition, disorder or disease, and/or comprises a nucleotide sequence associated with a condition, disorder or disease; and
administering to a subject susceptible to or suffering from a condition, disorder or disease an effective amount of an agent or a composition for preventing or treating the condition, disorder or disease; or
a method, comprising:
administering to a subject susceptible to or suffering from a condition, disorder or disease an effective amount of an agent or a composition for preventing or treating the condition, disorder or disease, wherein the agent or composition is assessed in a cell, tissue or animal, wherein the cell, tissue or animal is or comprises a cell, tissue or organ associated or of a condition, disorder or disease, and/or comprises a nucleotide sequence associated with a condition, disorder or disease.

94. The method of claim 93, wherein the subject is a human, and/or wherein a condition, disorder or disease is associated with a G to A mutation, and/or wherein a condition, disorder or disease is associated with 1024 G>A (E342K) mutation in human SERPINA1 gene, and/or wherein a condition, disorder or disease is alpha-1 antitrypsin deficiency.

95. A method, comprising
administering an agent or a composition thereof to a cell or a population thereof; and
assessing editing of an adenosine in a transcript in a cell or a population thereof; or
a method, comprising
administering an agent or a composition thereof to an animal or a population thereof; and
assessing editing of an adenosine in a transcript in one or more cells or tissues of the animal or a population thereof; or
a method, comprising
administering an oligonucleotide composition to a cell or a population thereof, and
assessing editing of an adenosine in a transcript in a cell or a population thereof; or
a method, comprising
administering an oligonucleotide composition to an animal or a population thereof; and
assessing editing of an adenosine in a transcript in one or more cells or tissues of the animal or a population thereof.

96. The method of claim 95, wherein the oligonucleotide composition is a chirally controlled oligonucleotide composition.

97. The method of any one of claims 95-96, wherein the cell, animal, or a population thereof is of any one of the preceding claims.

98. The method of any one of claims 93-97, comprising obtaining an agent, a composition thereof or an oligonucleotide composition after an assessing step on a commercial scale and/or for administration to a human and/or as a drug product.

99. The method of any one of claims 93-98, comprising:
administering a sample from a commercial batch or a drug product of an agent, a composition thereof or an oligonucleotide composition a cell or animal, or a population thereof,
assessing editing an adenosine in a transcript in the cell, or one or more cells or tissues of the animal, or a population thereof.

100. The method of claim 99, wherein editing of the adenosine is at a level comparable to that of a non-commercial production prior to the first commercial production, and/or wherein editing of the adenosine is at a level comparable to that of another batch of commercial production or drug product, and/or wherein editing of the adenosine is at a level comparable to that of a reference sample or drug product, and/or wherein editing of the adenosine is at a level that is within a reference range, and/or comprising releasing the commercial batch or drug product for delivery, distribution or administration.

101. The method of claim 99, wherein editing of the adenosine is at a level not comparable to that of a non-commercial production prior to the first commercial production, and/or wherein editing of the adenosine is at a level not comparable to that of another batch of commercial production or drug product, and/or wherein editing of the adenosine is at a level not comparable to that of a reference sample or drug product, and/or wherein editing of the adenosine is at a level that is not within a reference range, and/or comprising rejecting the commercial batch or drug product for delivery, distribution or administration.

102. A cell, animal, embryo, polynucleotide, vector, polypeptide or method described in the specification or of any one Embodiments 1-435.
